(19) United States
(12) Patent Application Publication
(10) Pub. No.: US 2026/0108603 A1
(43) Pub. Date: Apr. 23, 2026

(54) ANTIBODIES THAT BIND OX40L IN COMBINATION WITH HYALURONIDASE OR A VARIANT THEREOF

(71) Applicant: Apogee Therapeutics, Inc., Waltham, MA (US)

(72) Inventors: Aaron NOYES, Apex, NC (US); Rebecca Lucille DABORA, Bethlehem, PA (US)

(21) Appl. No.: 19/361,800

(22) Filed: Oct. 17, 2025

Related U.S. Application Data
(60) Provisional application No. 63/708,872, filed on Oct. 18, 2024.

(51) Int. Cl. A61K 39/395; A61K 38/47

(52) U.S. Cl. CPC A61K 39/3955; A61K 38/47; C12Y 302/01035

(57) ABSTRACT

Described herein are compositions and combinations comprising an antibody that binds OX40L and a hyaluronidase or variant thereof, as well as related methods of treating an inflammatory disorder or disease in a human patient.

### CROSS REFERENCE TO RELATED APPLICATIONS

This application claims the benefit of U.S. Provisional Application No. 63/708,872 (filed Oct. 18, 2024), which is incorporated by reference herein in its entirety.

### SEQUENCE LISTING

The instant application contains a Sequence Listing which has been submitted electronically in XML file format and is hereby incorporated by reference in its entirety. Said XML copy, created on Oct. 17, 2025, is named AOJ-007_SL.xml and is 1,052,282 bytes in size.

### BACKGROUND

An emerging mechanism in treatments for atopic dermatitis (AD) is targeting OX40 or OX40L. OX40L is the ligand for OX40. OX40L is expressed on antigen presenting cells and its interaction with OX40 causes the accumulation of T cells by providing a survival signal. T cells are important types of white blood cells of the immune system that play a central role in the immune response. OX40L, by playing a role in activating T cells and reprogramming them into inflammatory subsets, contributes to immune overactivation in AD and other inflammatory conditions, such as Systemic Lupus Erythematosus. Additionally, OX40L activation of OX40 inhibits the expression of FOXP3 and the inhibitory function of regulatory T (Treg) cells. Treg cells can suppress the immune response, which leads to worsening symptoms in inflammatory conditions. Therefore, OX40L blockade may lead to clinical benefit in AD and other inflammatory conditions by first suppressing inflammatory T cell activation, and next by increasing the proliferation of Treg cells, which can serve to further reduce inflammatory cells. Amlitelimab, which targets OX40L, and rocatinlimab, which targets OX40, have both demonstrated promising Phase 2 data in AD.

There is a need in the art for improved therapeutics that target OX40L, such as recombinant antibodies, alone or in combination with additional therapeutic agents. Accordingly, it is an object of the present invention to provide improved methods for treating patients with inflammatory diseases and conditions. There is also a need for delivering relatively large doses of these antibodies subcutaneously.

### SUMMARY

The present invention features compositions and combinations comprising an anti-OX40L antibody and a hyaluronidase or a variant thereof, and methods of treating an inflammatory disorder or disease by administering to a subject in need thereof an anti-OX40L antibody and a hyaluronidase or a variant thereof. Diseases that can be treated according to the methods described herein include atopic dermatitis and asthma, among others.

In a first aspect, disclosed herein are compositions comprising a hyaluronidase or a variant thereof and antibodies or antigen binding fragments thereof, such as recombinant human antibodies, that specifically bind human OX40L.

In another aspect, the invention provides a composition comprising an isolated antibody, or antigen binding fragment thereof, that binds OX40L and a hyaluronidase or a variant thereof, wherein the isolated antibody, or antigen binding fragment thereof, that binds OX40L comprises: a) a variable heavy (VH) chain sequence having three heavy chain CDR sequences, CDR-H1, CDR-H2, and CDR-H3; and b) a variable light (VL) chain sequence having three light chain CDR sequences, CDR-L1, CDR-L2, and CDR-L3; wherein: a. CDR-H1 comprises an amino acid sequence set forth in any one of SEQ ID NOs: 1-69 and 740-747; b. CDR-H2 comprises an amino acid sequence set forth in any one of SEQ ID NOs: 70-174 and 748-762; c. CDR-H3 comprises an amino acid sequence set forth in any one of SEQ ID NOs: 175-257 and 763-780, d. CDR-L1 comprises an amino acid sequence set forth in any one of SEQ ID NOs: 258-334 and 781-796, e. CDR-L2 comprises an amino acid sequence set forth in any one of SEQ ID NOs: 335-367 and 797-805; and f. CDR-L3 comprises an amino acid sequence set forth in any one of SEQ ID NOs: 268-393 and 806-811.

In another aspect, the invention provides a combination comprising an isolated antibody, or antigen binding fragment thereof, that binds OX40L and a hyaluronidase or a variant thereof, wherein the isolated antibody, or antigen binding fragment thereof, that binds OX40L comprises: a) a variable heavy (VH) chain sequence having three heavy chain CDR sequences, CDR-H1, CDR-H2, and CDR-H3; and b) a variable light (VL) chain sequence having three light chain CDR sequences, CDR-L1, CDR-L2, and CDR-L3; wherein: a. CDR-H1 comprises an amino acid sequence set forth in any one of SEQ ID NOs: 1-69 and 740-747; b. CDR-H2 comprises an amino acid sequence set forth in any one of SEQ ID NOs: 70-174 and 748-762; c. CDR-H3 comprises an amino acid sequence set forth in any one of SEQ ID NOs: 175-257 and 763-780, d. CDR-L1 comprises an amino acid sequence set forth in any one of SEQ ID NOs: 258-334 and 781-796, e. CDR-L2 comprises an amino acid sequence set forth in any one of SEQ ID NOs: 335-367 and 797-805; and f. CDR-L3 comprises an amino acid sequence set forth in any one of SEQ ID NOs: 268-393 and 806-811.

In another aspect, provided herein is a method of treating an inflammatory disorder or disease in a human patient in need thereof comprising administering to the patient an isolated antibody, or antigen binding fragment thereof, that binds OX40L and a hyaluronidase or a variant thereof, wherein the antibody, or antigen binding fragment thereof, that binds OX40L comprises: a) a variable heavy (VH) chain sequence having three heavy chain CDR sequences, CDR-H1, CDR-H2, and CDR-H3; and b) a variable light (VL) chain sequence having three light chain CDR sequences, CDR-L1, CDR-L2, and CDR-L3; wherein: a. CDR-H1 comprises an amino acid sequence set forth in any one of SEQ ID NOs: 1-69 and 740-747; b. CDR-H2 comprises an amino acid sequence set forth in any one of SEQ ID NOs: 70-174 and 748-762; c. CDR-H3 comprises an amino acid sequence set forth in any one of SEQ ID NOs: 175-257 and 763-780, d. CDR-L1 comprises an amino acid sequence set forth in any one of SEQ ID NOs: 258-334 and 781-796, e. CDR-L2 comprises an amino acid sequence set forth in any one of SEQ ID NOs: 335-367 and 797-805; and f. CDR-L3 comprises an amino acid sequence set forth in any one of SEQ ID NOs: 268-393 and 806-811.

In some embodiments, an antibody, or antigen binding fragment thereof, provided herein comprises a CDR-H1 comprising an amino acid sequence set forth in any one of SEQ ID NOs: 1, 22, and 44; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 258, 286, and 311; a CDR-L2 comprising an amino acid sequence set forth in any one of SEQ ID NOs: 335 and 359; and a CDR-L3 comprising an amino acid sequence set forth in any one of SEQ ID NOs: 368 and 394.

In some embodiments, an antibody, or antigen binding fragment thereof, provided herein comprises a CDR-H1 comprising an amino acid sequence set forth in any one of SEQ ID NOs: 2, 23, and 45; a CDR-H2 comprising an amino acid sequence set forth in any one of SEQ ID NOs: 70, 111, and 142; a CDR-H3 comprising an amino acid sequence set forth in any one of SEQ ID NOs: 176, 204, and 224; a CDR-L1 comprising an amino acid sequence set forth in any one of SEQ ID NOs: 259, 287, and 312; a CDR-L2 comprising an amino acid sequence set forth in any one of SEQ ID NOs: 336 and 360; and a CDR-L3 comprising an amino acid sequence set forth in any one of SEQ ID NOs: 369 and 395.

In some embodiments, an antibody, or antigen binding fragment thereof, provided herein comprises a CDR-H1 comprising an amino acid sequence set forth in any one of SEQ ID NOs: 3, 24, and 46; a CDR-H2 comprising an amino acid sequence set forth in any one of SEQ ID NOs: 71, 112, and 143; a CDR-H3 comprising an amino acid sequence set forth in any one of SEQ ID NOs: 177, 205, and 225; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 370 and 396.

In some embodiments, an antibody, or antigen binding fragment thereof, provided herein comprises a CDR-H1 comprising an amino acid sequence set forth in any one of SEQ ID NOs: 4, 22, and 47; a CDR-H2 comprising an amino acid sequence set forth in any one of SEQ ID NOs: 72, 113, and 144; a CDR-H3 comprising an amino acid sequence set forth in any one of SEQ ID NOs: 178, 206, and 226; a CDR-L1 comprising an amino acid sequence set forth in any one of SEQ ID NOs: 261, 289, and 314; a CDR-L2 comprising an amino acid sequence set forth in any one of SEQ ID NOs: 337 and 361; and a CDR-L3 comprising an amino acid sequence set forth in any one of SEQ ID NOs: 371 and 397.

In some embodiments, an antibody, or antigen binding fragment thereof, provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 73, 114, and 145; a CDR-H3 comprising an amino acid sequence set forth in any one of SEQ ID NOs: 179, 207, and 227; a CDR-L1 comprising an amino acid sequence set forth in any one of SEQ ID NOs: 262, 290, and 315; a CDR-L2 comprising an amino acid sequence set forth in any one of SEQ ID NOs: 338 and 362; and a CDR-L3 comprising an amino acid sequence set forth in any one of SEQ ID NOs: 372 or 398.

In some embodiments, an antibody, or antigen binding fragment thereof, provided herein comprises a CDR-H1 comprising an amino acid sequence set forth in any one of SEQ ID NOs: 6, 24, and 49; a CDR-H2 comprising an amino acid sequence set forth in any one of SEQ ID NOs: 74, 115, and 146; a CDR-H3 comprising an amino acid sequence set forth in any one of SEQ ID NOs: 180, 208, and 228; a CDR-L1 comprising an amino acid sequence set forth in any one of SEQ ID NOs: 263, 291, and 316; a CDR-L2 comprising an amino acid sequence set forth in any of one SEQ ID NOs: 339 and 361; and a CDR-L3 comprising an amino acid sequence set forth in any one of SEQ ID NOs: 373 and 399.

In some embodiments, an antibody, or antigen binding fragment thereof, provided herein comprises a CDR-H1 comprising an amino acid sequence set forth in any one of SEQ ID NOs: 7, 25, and 50; a CDR-H2 comprising an amino acid sequence set forth in any one of SEQ ID NOs: 75, 116, and 147; a CDR-H3 comprising an amino acid sequence set forth in any one of SEQ ID NOs: 181, 209, and 229; a CDR-L1 comprising an amino acid sequence set forth in any one of SEQ ID NOs: 264, 292, and 317; a CDR-L2 comprising an amino acid sequence set forth in any one of SEQ ID NOs: 340 and 363; and a CDR-L3 comprising an amino acid sequence set forth in any one of SEQ ID NOs: 374 and 400.

In some embodiments, an antibody, or antigen binding fragment thereof, provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 182, 210, and 230; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 375 and 401.

In some embodiments, an antibody, or antigen binding fragment thereof, provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 183, 211, and 231; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 376 and 402.

In some embodiments, an antibody, or antigen binding fragment thereof, provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 183, 211, and 231; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 377 and 403.

In some embodiments, an antibody, or antigen binding fragment thereof, provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 184, 212, and 232; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 376 and 402.

In some embodiments, an antibody, or antigen binding fragment thereof, provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 184, 212, and 232; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 377 and 403.

In some embodiments, an antibody, or antigen binding fragment thereof, provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 185, 213, and 233; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 376 and 402.

In some embodiments, an antibody, or antigen binding fragment thereof, provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 185, 213, and 233; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 377 and 403.

In some embodiments, an antibody, or antigen binding fragment thereof, provided herein comprises a CDR-H1 comprising an amino acid sequence set forth in any one of SEQ ID NOs: 9, 27, and 52; a CDR-H2 comprising an amino acid sequence set forth in any one of SEQ ID NOs: 77, 118, and 149; a CDR-H3 comprising an amino acid sequence set forth in any one of SEQ ID NOs: 175, 203, and 234; a CDR-L1 comprising an amino acid sequence set forth in any one of SEQ ID NOs: 260, 288, and 313; a CDR-L2 comprising an amino acid sequence set forth in any one of SEQ ID NOs: 342 and 364; and a CDR-L3 comprising an amino acid sequence set forth in any one of SEQ ID NOs: 368 and 394.

In some embodiments, an antibody, or antigen binding fragment thereof, provided herein comprises a CDR-H1 comprising an amino acid sequence set forth in any one of SEQ ID NOs: 1, 28, and 53; a CDR-H2 comprising an amino acid sequence set forth in any one of SEQ ID NOs: 78, 119, and 150; a CDR-H3 comprising an amino acid sequence set forth in any one of SEQ ID NOs: 175, 203, and 223; a CDR-L1 comprising an amino acid sequence set forth in any one of SEQ ID NOs: 266, 286, and 311; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 79, 119, and 150; a CDR-H3 comprising an amino acid sequence set forth in any one of SEQ ID NOs: 186, 203, and 235; a CDR-L1 comprising an amino acid sequence set forth in any one of SEQ ID NOs: 267, 294, and 319; a CDR-L2 comprising an amino acid sequence set forth in any one of SEQ ID NOs: 344 and 365; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 80, 120, and 151; a CDR-H3 comprising an amino acid sequence set forth in any one of SEQ ID NOs: 186, 203, and 235; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 11, 27, and 52; a CDR-H2 comprising an amino acid sequence set forth in any one of SEQ ID NOs: 79, 119, and 150; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 11, 27, and 52; a CDR-H2 comprising an amino acid sequence set forth in any one of SEQ ID NOs: 81, 121, and 152; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 80, 120, and 151; a CDR-H3 comprising an amino acid sequence set forth in any one of SEQ ID NOs: 187, 203, and 236; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 79, 119, and 150; a CDR-H3 comprising an amino acid sequence set forth in any one of SEQ ID NOs: 187, 203, and 236; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 188, 203, and 237; a CDR-L1 comprising an amino acid sequence set forth in any one of SEQ ID NOs: 268, 288, and 313; a CDR-L2 comprising an amino acid sequence set forth in any one of SEQ ID NOs: 337 and 361; and a CDR-L3 comprising an amino acid sequence set forth in any one of SEQ ID NOs: 368 and 394.

In some embodiments, an antibody, or antigen binding fragment thereof, provided herein comprises a CDR-H1 comprising an amino acid sequence set forth in any one of SEQ ID NOs: 12, 29, and 54; a CDR-H2 comprising an amino acid sequence set forth in any one of SEQ ID NOs: 70, 111, and 142; a CDR-H3 comprising an amino acid sequence set forth in any one of SEQ ID NOs: 188, 203, and 237; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 82, 111, and 142; a CDR-H3 comprising an amino acid sequence set forth in any one of SEQ ID NOs: 189, 214, and 238; a CDR-L1 comprising an amino acid sequence set forth in any one of SEQ ID NOs: 269, 288, and 313; a CDR-L2 comprising an amino acid sequence set forth in any one of SEQ ID NOs: 345 and 361; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 83, 122, and 153; a CDR-H3 comprising an amino acid sequence set forth in any one of SEQ ID NOs: 175, 203, and 234; a CDR-L1 comprising an amino acid sequence set forth in any one of SEQ ID NOs: 260, 288, and 313; a CDR-L2 comprising an amino acid sequence set forth in any one of SEQ ID NOs: 346 and 361; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 13, 30, and 55; a CDR-H2 comprising an amino acid sequence set forth in any one of SEQ ID NOs: 84, 111, and 142; a CDR-H3 comprising an amino acid sequence set forth in any one of SEQ ID NOs: 188, 203, and 239; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 234; a CDR-L1 comprising an amino acid sequence set forth in any one of SEQ ID NOs: 260, 288, and 313; a CDR-L2 comprising an amino acid sequence set forth in any one of SEQ ID NOs: 347 and 365; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 234; a CDR-L1 comprising an amino acid sequence set forth in any one of SEQ ID NOs: 260, 288, and 313; a CDR-L2 comprising an amino acid sequence set forth in any one of SEQ ID NOs: 345 and 361; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 85, 123, and 154; a CDR-H3 comprising an amino acid sequence set forth in any one of SEQ ID NOs: 188, 203, and 239; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 270, 295, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 1, 31, and 56; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 379 and 405.

In some embodiments, an antibody, or antigen binding fragment thereof, provided herein comprises a CDR-H1 comprising an amino acid sequence set forth in any one of SEQ ID NOs: 1, 32, and 57; a CDR-H2 comprising an amino acid sequence set forth in any one of SEQ ID NOs: 86, 124, and 155; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 380 and 406.

In some embodiments, an antibody, or antigen binding fragment thereof, provided herein comprises a CDR-H1 comprising an amino acid sequence set forth in any one of SEQ ID NOs: 14, 33, and 58; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 87, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 87, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 13, 30, and 55; a CDR-H2 comprising an amino acid sequence set forth in any one of SEQ ID NOs: 87, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 368 and 394.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 87, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 348 and 366; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 381 and 405.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 335 and 359; and a CDR-L3 comprising an amino acid sequence set forth in any one of SEQ ID NOs: 368 and 394.

In some embodiments, an antibody, or antigen binding fragment thereof, provided herein comprises a CDR-H1 comprising an amino acid sequence set forth in any one of SEQ ID NOs: 1, 22, and 44; a CDR-H2 comprising an amino acid sequence set forth in any one of SEQ ID NOs: 86, 124, and 155; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 381 and 405.

In some embodiments, an antibody, or antigen binding fragment thereof, provided herein comprises a CDR-H1 comprising an amino acid sequence set forth in any one of SEQ ID NOs: 1, 22, and 44; a CDR-H2 comprising an amino acid sequence set forth in any one of SEQ ID NOs: 86, 124, and 155; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 188, 203, and 239; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 188, 203, and 237; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 78, 119, and 150; a CDR-H3 comprising an amino acid sequence set forth in any one of SEQ ID NOs: 188, 203, and 237; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 368 and 394.

In some embodiments, an antibody, or antigen binding fragment thereof, provided herein comprises a CDR-H1 comprising an amino acid sequence set forth in any one of SEQ ID NOs: 2, 840, and 45; a CDR-H2 comprising an amino acid sequence set forth in any one of SEQ ID NOs: 70, 111, and 142; a CDR-H3 comprising an amino acid sequence set forth in any one of SEQ ID NOs: 176, 204, and 224; a CDR-L1 comprising an amino acid sequence set forth in any one of SEQ ID NOs: 259, 287, and 312; a CDR-L2 comprising an amino acid sequence set forth in any one of SEQ ID NOs: 336 and 360; and a CDR-L3 comprising an amino acid sequence set forth in any one of SEQ ID NOs: 369 and 395.

In some embodiments, an antibody, or antigen binding fragment thereof, provided herein comprises a CDR-H1 comprising an amino acid sequence set forth in any one of SEQ ID NOs: 15, 34, and 59; a CDR-H2 comprising an amino acid sequence set forth in any one of SEQ ID NOs: 88, 125, and 156; a CDR-H3 comprising an amino acid sequence set forth in any one of SEQ ID NOs: 190, 215, and 240; a CDR-L1 comprising an amino acid sequence set forth in any one of SEQ ID NOs: 269, 288, and 313; a CDR-L2 comprising an amino acid sequence set forth in any one of SEQ ID NOs: 349 and 365; and a CDR-L3 comprising an amino acid sequence set forth in any one of SEQ ID NOs: 382 and 407.

In some embodiments, an antibody, or antigen binding fragment thereof, provided herein comprises a CDR-H1 comprising an amino acid sequence set forth in any one of SEQ ID NOs: 4, 22, and 47; a CDR-H2 comprising an amino acid sequence set forth in any one of SEQ ID NOs: 89, 126, and 157; a CDR-H3 comprising an amino acid sequence set forth in any one of SEQ ID NOs: 178, 206, and 226; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 371 and 397.

In some embodiments, an antibody, or antigen binding fragment thereof, provided herein comprises a CDR-H1 comprising an amino acid sequence set forth in any one of SEQ ID NOs: 4, 35, and 60; a CDR-H2 comprising an amino acid sequence set forth in any one of SEQ ID NOs: 72, 113, and 144; a CDR-H3 comprising an amino acid sequence set forth in any one of SEQ ID NOs: 178, 206, and 241; a CDR-L1 comprising an amino acid sequence set forth in any one of SEQ ID NOs: 271, 296, and 320; a CDR-L2 comprising an amino acid sequence set forth in any one of SEQ ID NOs: 350 and 361; and a CDR-L3 comprising an amino acid sequence set forth in any one of SEQ ID NOs: 371 and 397.

In some embodiments, an antibody, or antigen binding fragment thereof, provided herein comprises a CDR-H1 comprising an amino acid sequence set forth in any one of SEQ ID NOs: 4, 22, and 47; a CDR-H2 comprising an amino acid sequence set forth in any one of SEQ ID NOs: 90, 127, and 158; a CDR-H3 comprising an amino acid sequence set forth in any one of SEQ ID NOs: 178, 206, and 226; a CDR-L1 comprising an amino acid sequence set forth in any one of SEQ ID NOs: 261, 289, and 314; a CDR-L2 comprising an amino acid sequence set forth in any one of SEQ ID NOs: 337 and 361; and a CDR-L3 comprising an amino acid sequence set forth in any one of SEQ ID NOs: 371 and 397.

In some embodiments, an antibody, or antigen binding fragment thereof, provided herein comprises a CDR-H1 comprising an amino acid sequence set forth in any one of SEQ ID NOs: 16, 36, and 61; a CDR-H2 comprising an amino acid sequence set forth in any one of SEQ ID NOs: 91, 128, and 159; a CDR-H3 comprising an amino acid sequence set forth in any one of SEQ ID NOs: 191, 215, and 242; a CDR-L1 comprising an amino acid sequence set forth in any one of SEQ ID NOs: 269, 288, and 313; a CDR-L2 comprising an amino acid sequence set forth in any one of SEQ ID NOs: 351 and 361; and a CDR-L3 comprising an amino acid sequence set forth in any one of SEQ ID NOs: 382 and 407.

In some embodiments, an antibody, or antigen binding fragment thereof, provided herein comprises a CDR-H1 comprising an amino acid sequence set forth in any one of SEQ ID NOs: 16, 36, and 61; a CDR-H2 comprising an amino acid sequence set forth in any one of SEQ ID NOs: 91, 128, and 159; a CDR-H3 comprising an amino acid sequence set forth in any one of SEQ ID NOs: 190, 215, and 240; a CDR-L1 comprising an amino acid sequence set forth in any one of SEQ ID NOs: 269, 288, and 313; a CDR-L2 comprising an amino acid sequence set forth in any one of SEQ ID NOs: 351 and 361; and a CDR-L3 comprising an amino acid sequence set forth in any one of SEQ ID NOs: 371 and 397.

In some embodiments, an antibody, or antigen binding fragment thereof, provided herein comprises a CDR-H1 comprising an amino acid sequence set forth in any one of SEQ ID NOs: 15, 27, and 62; a CDR-H2 comprising an amino acid sequence set forth in any one of SEQ ID NOs: 72, 113, and 144; a CDR-H3 comprising an amino acid sequence set forth in any one of SEQ ID NOs: 190, 215, and 240; a CDR-L1 comprising an amino acid sequence set forth in any one of SEQ ID NOs: 269, 288, and 313; a CDR-L2 comprising an amino acid sequence set forth in any one of SEQ ID NOs: 337 and 361; and a CDR-L3 comprising an amino acid sequence set forth in any one of SEQ ID NOs: 382 and 407.

In some embodiments, an antibody, or antigen binding fragment thereof, provided herein comprises a CDR-H1 comprising an amino acid sequence set forth in any one of SEQ ID NOs: 15, 27, and 62; a CDR-H2 comprising an amino acid sequence set forth in any one of SEQ ID NOs: 72, 113, and 144; a CDR-H3 comprising an amino acid sequence set forth in any one of SEQ ID NOs: 190, 215, and 240; a CDR-L1 comprising an amino acid sequence set forth in any one of SEQ ID NOs: 269, 288, and 313; a CDR-L2 comprising an amino acid sequence set forth in any one of SEQ ID NOs: 351 and 361; and a CDR-L3 comprising an amino acid sequence set forth in any one of SEQ ID NOs: 383 and 408.

In some embodiments, an antibody, or antigen binding fragment thereof, provided herein comprises a CDR-H1 comprising an amino acid sequence set forth in any one of SEQ ID NOs: 5, 37, and 63; a CDR-H2 comprising an amino acid sequence set forth in any one of SEQ ID NOs: 92, 129, and 160; a CDR-H3 comprising an amino acid sequence set forth in any one of SEQ ID NOs: 179, 207, and 227; a CDR-L1 comprising an amino acid sequence set forth in any one of SEQ ID NOs: 272, 297, and 321; a CDR-L2 comprising an amino acid sequence set forth in any one of SEQ ID NOs: 338 and 362; and a CDR-L3 comprising an amino acid sequence set forth in any one of SEQ ID NOs: 384 and 409.

In some embodiments, an antibody, or antigen binding fragment thereof, provided herein comprises a CDR-H1 comprising an amino acid sequence set forth in any one of SEQ ID NOs: 5, 24, and 49; a CDR-H2 comprising an amino acid sequence set forth in any one of SEQ ID NOs: 93, 130, and 161; a CDR-H3 comprising an amino acid sequence set forth in any one of SEQ ID NOs: 179, 207, and 227; a CDR-L1 comprising an amino acid sequence set forth in any one of SEQ ID NOs: 272, 297, and 321; a CDR-L2 comprising an amino acid sequence set forth in any one of SEQ ID NOs: 338 and 362; and a CDR-L3 comprising an amino acid sequence set forth in any one of SEQ ID NOs: 384 and 409.

In some embodiments, an antibody, or antigen binding fragment thereof, provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 94, 131, and 162; a CDR-H3 comprising an amino acid sequence set forth in any one of SEQ ID NOs: 192, 207, and 243; a CDR-L1 comprising an amino acid sequence set forth in any one of SEQ ID NOs: 273, 298, and 322; a CDR-L2 comprising an amino acid sequence set forth in any one of SEQ ID NOs: 338 and 362; and a CDR-L3 comprising an amino acid sequence set forth in any one of SEQ ID NOs: 385 and 410.

In some embodiments, an antibody, or antigen binding fragment thereof, provided herein comprises a CDR-H1 comprising an amino acid sequence set forth in any one of SEQ ID NOs: 17, 38, and 64; a CDR-H2 comprising an amino acid sequence set forth in any one of SEQ ID NOs: 95, 132, and 163; a CDR-H3 comprising an amino acid sequence set forth in any one of SEQ ID NOs: 179, 207, and 227; a CDR-L1 comprising an amino acid sequence set forth in any one of SEQ ID NOs: 272, 297, and 321; a CDR-L2 comprising an amino acid sequence set forth in any one of SEQ ID NOs: 338 and 362; and a CDR-L3 comprising an amino acid sequence set forth in any one of SEQ ID NOs: 386 and 411.

In some embodiments, an antibody, or antigen binding fragment thereof, provided herein comprises a CDR-H1 comprising an amino acid sequence set forth in any one of SEQ ID NOs: 5, 24, and 49; a CDR-H2 comprising an amino acid sequence set forth in any one of SEQ ID NOs: 96, 130, and 164; a CDR-H3 comprising an amino acid sequence set forth in any one of SEQ ID NOs: 192, 207, and 243; a CDR-L1 comprising an amino acid sequence set forth in any one of SEQ ID NOs: 274, 299, and 323; a CDR-L2 comprising an amino acid sequence set forth in any one of SEQ ID NOs: 338 and 362; and a CDR-L3 comprising an amino acid sequence set forth in any one of SEQ ID NOs: 386 and 411.

In some embodiments, an antibody, or antigen binding fragment thereof, provided herein comprises a CDR-H1 comprising an amino acid sequence set forth in any one of SEQ ID NOs: 5, 24, and 49; a CDR-H2 comprising an amino acid sequence set forth in any one of SEQ ID NOs: 97, 130, and 164; a CDR-H3 comprising an amino acid sequence set forth in any one of SEQ ID NOs: 179, 207, and 227; a CDR-L1 comprising an amino acid sequence set forth in any one of SEQ ID NOs: 275, 300, and 324; a CDR-L2 comprising an amino acid sequence set forth in any one of SEQ ID NOs: 338 and 362; and a CDR-L3 comprising an amino acid sequence set forth in any one of SEQ ID NOs: 386 and 411.

In some embodiments, an antibody, or antigen binding fragment thereof, provided herein comprises a CDR-H1 comprising an amino acid sequence set forth in any one of SEQ ID NOs: 18, 39, and 65; a CDR-H2 comprising an amino acid sequence set forth in any one of SEQ ID NOs: 98, 116, and 147; a CDR-H3 comprising an amino acid sequence set forth in any one of SEQ ID NOs: 193, 216, and 244; a CDR-L1 comprising an amino acid sequence set forth in any one of SEQ ID NOs: 276, 301, and 325; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, an antibody, or antigen binding fragment thereof, provided herein comprises a CDR-H1 comprising an amino acid sequence set forth in any one of SEQ ID NOs: 18, 39, and 65; a CDR-H2 comprising an amino acid sequence set forth in any one of SEQ ID NOs: 98, 116, and 147; a CDR-H3 comprising an amino acid sequence set forth in any one of SEQ ID NOs: 193, 216, and 245; a CDR-L1 comprising an amino acid sequence set forth in any one of SEQ ID NOs: 277, 302, and 326; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, an antibody, or antigen binding fragment thereof, provided herein comprises a CDR-H1 comprising an amino acid sequence set forth in any one of SEQ ID NOs: 18, 39, and 65; a CDR-H2 comprising an amino acid sequence set forth in any one of SEQ ID NOs: 98, 116, and 147; a CDR-H3 comprising an amino acid sequence set forth in any one of SEQ ID NOs: 194, 217, and 246; a CDR-L1 comprising an amino acid sequence set forth in any one of SEQ ID NOs: 276, 301, and 325; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, an antibody, or antigen binding fragment thereof, provided herein comprises a CDR-H1 comprising an amino acid sequence set forth in any one of SEQ ID NOs: 18, 39, and 65; a CDR-H2 comprising an amino acid sequence set forth in any one of SEQ ID NOs: 98, 116, and 147; a CDR-H3 comprising an amino acid sequence set forth in any one of SEQ ID NOs: 194, 217, and 247; a CDR-L1 comprising an amino acid sequence set forth in any one of SEQ ID NOs: 276, 301, and 325; a CDR-L2 comprising an amino acid sequence set forth in any one of SEQ ID NOs: 337 and 361; and a CDR-L3 comprising an amino acid sequence set forth in any one of SEQ ID NOs: 388 and 413.

In some embodiments, an antibody, or antigen binding fragment thereof, provided herein comprises a CDR-H1 comprising an amino acid sequence set forth in any one of SEQ ID NOs: 19, 40, and 66; a CDR-H2 comprising an amino acid sequence set forth in any one of SEQ ID NOs: 99, 133, and 165; a CDR-H3 comprising an amino acid sequence set forth in any one of SEQ ID NOs: 180, 208, and 248; a CDR-L1 comprising an amino acid sequence set forth in any one of SEQ ID NOs: 278, 303, and 327; a CDR-L2 comprising an amino acid sequence set forth in any one of SEQ ID NOs: 352 and 367; and a CDR-L3 comprising an amino acid sequence set forth in any one of SEQ ID NOs: 387 and 412.

In some embodiments, an antibody, or antigen binding fragment thereof, provided herein comprises a CDR-H1 comprising an amino acid sequence set forth in any one of SEQ ID NOs: 20, 41, and 67; a CDR-H2 comprising an amino acid sequence set forth in any one of SEQ ID NOs: 100, 134, and 166; a CDR-H3 comprising an amino acid sequence set forth in any one of SEQ ID NOs: 195, 218, and 249; a CDR-L1 comprising an amino acid sequence set forth in any one of SEQ ID NOs: 279, 304, and 328; a CDR-L2 comprising an amino acid sequence set forth in any one of SEQ ID NOs: 353 and 361; and a CDR-L3 comprising an amino acid sequence set forth in any one of SEQ ID NOs: 374 and 400.

In some embodiments, an antibody, or antigen binding fragment thereof, provided herein comprises a CDR-H1 comprising an amino acid sequence set forth in any one of SEQ ID NOs: 20, 840, and 48; a CDR-H2 comprising an amino acid sequence set forth in any one of SEQ ID NOs: 101, 133, and 167; a CDR-H3 comprising an amino acid sequence set forth in any one of SEQ ID NOs: 180, 208, and 248; a CDR-L1 comprising an amino acid sequence set forth in any one of SEQ ID NOs: 280, 305, and 329; a CDR-L2 comprising an amino acid sequence set forth in any one of SEQ ID NOs: 337 and 361; and a CDR-L3 comprising an amino acid sequence set forth in any one of SEQ ID NOs: 389 and 414.

In some embodiments, an antibody, or antigen binding fragment thereof, provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 102, 135, and 168; a CDR-H3 comprising an amino acid sequence set forth in any one of SEQ ID NOs: 180, 208, and 248; a CDR-L1 comprising an amino acid sequence set forth in any one of SEQ ID NOs: 280, 305, and 329; a CDR-L2 comprising an amino acid sequence set forth in any one of SEQ ID NOs: 350 and 361; and a CDR-L3 comprising an amino acid sequence set forth in any one of SEQ ID NOs: 389 and 414.

In some embodiments, an antibody, or antigen binding fragment thereof, provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 103, 136, and 169; a CDR-H3 comprising an amino acid sequence set forth in any one of SEQ ID NOs: 196, 218, and 250; a CDR-L1 comprising an amino acid sequence set forth in any one of SEQ ID NOs: 280, 305, and 329; a CDR-L2 comprising an amino acid sequence set forth in any one of SEQ ID NOs: 354 and 367; and a CDR-L3 comprising an amino acid sequence set forth in any one of SEQ ID NOs: 390 and 415.

In some embodiments, an antibody, or antigen binding fragment thereof, provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 104, 137, and 170; a CDR-H3 comprising an amino acid sequence set forth in any one of SEQ ID NOs: 197, 208, and 251; a CDR-L1 comprising an amino acid sequence set forth in any one of SEQ ID NOs: 281, 306, and 330; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, an antibody, or antigen binding fragment thereof, provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 105, 138, and 171; a CDR-H3 comprising an amino acid sequence set forth in any one of SEQ ID NOs: 197, 208, and 251; a CDR-L1 comprising an amino acid sequence set forth in any one of SEQ ID NOs: 281, 306, and 330; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, an antibody, or antigen binding fragment thereof, provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 94, 131, and 162; a CDR-H3 comprising an amino acid sequence set forth in any one of SEQ ID NOs: 198, 219, and 252; a CDR-L1 comprising an amino acid sequence set forth in any one of SEQ ID NOs: 282, 307, and 331; a CDR-L2 comprising an amino acid sequence set forth in any one of SEQ ID NOs: 337 and 361; and a CDR-L3 comprising an amino acid sequence set forth in any one of SEQ ID NOs: 391 and 416.

In some embodiments, an antibody, or antigen binding fragment thereof, provided herein comprises a CDR-H1 comprising an amino acid sequence set forth in any one of SEQ ID NOs: 20, 840, and 48; a CDR-H2 comprising an amino acid sequence set forth in any one of SEQ ID NOs: 94, 131, and 162; a CDR-H3 comprising an amino acid sequence set forth in any one of SEQ ID NOs: 195, 218, and 253; a CDR-L1 comprising an amino acid sequence set forth in any one of SEQ ID NOs: 280, 305, and 329; a CDR-L2 comprising an amino acid sequence set forth in any one of SEQ ID NOs: 355 and 367; and a CDR-L3 comprising an amino acid sequence set forth in any one of SEQ ID NOs: 387 and 412.

In some embodiments, an antibody, or antigen binding fragment thereof, provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 94, 131, and 162; a CDR-H3 comprising an amino acid sequence set forth in any one of SEQ ID NOs: 180, 208, and 248; a CDR-L1 comprising an amino acid sequence set forth in any one of SEQ ID NOs: 280, 305, and 329; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, an antibody, or antigen binding fragment thereof, provided herein comprises a CDR-H1 comprising an amino acid sequence set forth in any one of SEQ ID NOs: 6, 840, and 48; a CDR-H2 comprising an amino acid sequence set forth in any one of SEQ ID NOs: 106, 137, and 170; a CDR-H3 comprising an amino acid sequence set forth in any one of SEQ ID NOs: 195, 218, and 253; a CDR-L1 comprising an amino acid sequence set forth in any one of SEQ ID NOs: 280, 305, and 329; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, an antibody, or antigen binding fragment thereof, provided herein comprises a CDR-H1 comprising an amino acid sequence set forth in any one of SEQ ID NOs: 20, 41, and 67; a CDR-H2 comprising an amino acid sequence set forth in any one of SEQ ID NOs: 107, 133, and 167; a CDR-H3 comprising an amino acid sequence set forth in any one of SEQ ID NOs: 180, 208, and 248; a CDR-L1 comprising an amino acid sequence set forth in any one of SEQ ID NOs: 280, 305, and 329; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, an antibody, or antigen binding fragment thereof, provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 108, 139, and 172; a CDR-H3 comprising an amino acid sequence set forth in any one of SEQ ID NOs: 181, 209, and 229; a CDR-L1 comprising an amino acid sequence set forth in any one of SEQ ID NOs: 283, 308, and 332; a CDR-L2 comprising an amino acid sequence set forth in any one of SEQ ID NOs: 356 and 361; and a CDR-L3 comprising an amino acid sequence set forth in any one of SEQ ID NOs: 374 and 400.

In some embodiments, an antibody, or antigen binding fragment thereof, provided herein comprises a CDR-H1 comprising an amino acid sequence set forth in any one of SEQ ID NOs: 21, 42, and 68; a CDR-H2 comprising an amino acid sequence set forth in any one of SEQ ID NOs: 109, 140, and 173; a CDR-H3 comprising an amino acid sequence set forth in any one of SEQ ID NOs: 199, 220, and 254; a CDR-L1 comprising an amino acid sequence set forth in any one of SEQ ID NOs: 280, 305, and 329; a CDR-L2 comprising an amino acid sequence set forth in any one of SEQ ID NOs: 337 and 361; and a CDR-L3 comprising an amino acid sequence set forth in any one of SEQ ID NOs: 374 and 400.

In some embodiments, an antibody, or antigen binding fragment thereof, provided herein comprises a CDR-H1 comprising an amino acid sequence set forth in any one of SEQ ID NOs: 5, 43, and 69; a CDR-H2 comprising an amino acid sequence set forth in any one of SEQ ID NOs: 106, 137, and 170; a CDR-H3 comprising an amino acid sequence set forth in any one of SEQ ID NOs: 200, 221, and 255; a CDR-L1 comprising an amino acid sequence set forth in any one of SEQ ID NOs: 284, 309, and 333; a CDR-L2 comprising an amino acid sequence set forth in any one of SEQ ID NOs: 337 and 361; and a CDR-L3 comprising an amino acid sequence set forth in any one of SEQ ID NOs: 374 and 400.

In some embodiments, an antibody, or antigen binding fragment thereof, provided herein comprises a CDR-H1 comprising an amino acid sequence set forth in any one of SEQ ID NOs: 5, 43, and 69; a CDR-H2 comprising an amino acid sequence set forth in any one of SEQ ID NOs: 94, 131, and 162; a CDR-H3 comprising an amino acid sequence set forth in any one of SEQ ID NOs: 201, 222, and 256; a CDR-L1 comprising an amino acid sequence set forth in any one of SEQ ID NOs: 285, 310, and 334; a CDR-L2 comprising an amino acid sequence set forth in any one of SEQ ID NOs: 357 and 361; and a CDR-L3 comprising an amino acid sequence set forth in any one of SEQ ID NOs: 392 and 417.

In some embodiments, an antibody, or antigen binding fragment thereof, provided herein comprises a CDR-H1 comprising an amino acid sequence set forth in any one of SEQ ID NOs: 5, 43, and 69; a CDR-H2 comprising an amino acid sequence set forth in any one of SEQ ID NOs: 110, 141, and 174; a CDR-H3 comprising an amino acid sequence set forth in any one of SEQ ID NOs: 202, 222, and 257; a CDR-L1 comprising an amino acid sequence set forth in any one of SEQ ID NOs: 285, 310, and 334; a CDR-L2 comprising an amino acid sequence set forth in any one of SEQ ID NOs: 358 and 361; and a CDR-L3 comprising an amino acid sequence set forth in any one of SEQ ID NOs: 393 and 418.

In some embodiments, an antibody, or antigen binding fragment thereof, provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 748, 116, and 758; a CDR-H3 comprising an amino acid sequence set forth in any one of SEQ ID NOs: 763, 769, and 775, a CDR-L1 comprising an amino acid sequence set forth in any one of SEQ ID NOs: 781, 787, and 792; a CDR-L2 comprising an amino acid sequence set forth in any one of SEQ ID NOs: 797 and 802; and a CDR-L3 comprising an amino acid sequence set forth in any one of SEQ ID NOs: 806 and 812.

In some embodiments, an antibody, or antigen binding fragment thereof, provided herein comprises a CDR-H1 comprising an amino acid sequence set forth in any one of SEQ ID NOs: 740, 742, and 745; a CDR-H2 comprising an amino acid sequence set forth in any one of SEQ ID NOs: 749, 754, and 759; a CDR-H3 comprising an amino acid sequence set forth in any one of SEQ ID NOs: 764, 770, and 776, a CDR-L1 comprising an amino acid sequence set forth in any one of SEQ ID NOs: 782, 788, and 793; a CDR-L2 comprising an amino acid sequence set forth in any one of SEQ ID NOs: 798 and 803; and a CDR-L3 comprising an amino acid sequence set forth in any one of SEQ ID NOs: 807 and 813.

In some embodiments, an antibody, or antigen binding fragment thereof, provided herein comprises a CDR-H1 comprising an amino acid sequence set forth in any one of SEQ ID NOs: 17, 743, and 746; a CDR-H2 comprising an amino acid sequence set forth in any one of SEQ ID NOs: 750, 133, and 165; a CDR-H3 comprising an amino acid sequence set forth in any one of SEQ ID NOs: 765, 771, and 777, a CDR-L1 comprising an amino acid sequence set forth in any one of SEQ ID NOs: 783, 298, and 322; a CDR-L2 comprising an amino acid sequence set forth in any one of SEQ ID NOs: 799 and 804; and a CDR-L3 comprising an amino acid sequence set forth in any one of SEQ ID NOs: 808 and 814.

In some embodiments, an antibody, or antigen binding fragment thereof, provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 751, 755, and 760; a CDR-H3 comprising an amino acid sequence set forth in any one of SEQ ID NOs: 766, 772, and 778, a CDR-L1 comprising an amino acid sequence set forth in any one of SEQ ID NOs: 784, 789, and 794; a CDR-L2 comprising an amino acid sequence set forth in any one of SEQ ID NOs: 341 and 361; and a CDR-L3 comprising an amino acid sequence set forth in any one of SEQ ID NOs: 809 and 815.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223, a CDR-L1 comprising an amino acid sequence set forth in any one of SEQ ID NOs: 260, 288, and 313; a CDR-L2 comprising an amino acid sequence set forth in any one of SEQ ID NOs: 341 and 361; and a CDR-L3 comprising an amino acid sequence set forth in any one of SEQ ID NOs: 368 and 394.

In some embodiments, an antibody, or antigen binding fragment thereof, provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 752, 756, and 761; a CDR-H3 comprising an amino acid sequence set forth in any one of SEQ ID NOs: 767, 773, and 779, a CDR-L1 comprising an amino acid sequence set forth in any one of SEQ ID NOs: 785, 790, and 795; a CDR-L2 comprising an amino acid sequence set forth in any one of SEQ ID NOs: 800 and 805; and a CDR-L3 comprising an amino acid sequence set forth in SEQ ID NOs: 810 and 816.

In some embodiments, an antibody, or antigen binding fragment thereof, provided herein comprises a CDR-H1 comprising an amino acid sequence set forth in any one of SEQ ID NOs: 741, 744, and 747; a CDR-H2 comprising an amino acid sequence set forth in any one of SEQ ID NOs: 753, 757, and 762; a CDR-H3 comprising an amino acid sequence set forth in SEQ ID NOs: 768, 774, and 780, a CDR-L1 comprising an amino acid sequence set forth in any one of SEQ ID NOs: 786, 791, and 796; a CDR-L2 comprising an amino acid sequence set forth in any one of SEQ ID NOs: 801 and 360; and a CDR-L3 comprising an amino acid sequence set forth in SEQ ID NOs: 811 and 817.

In some embodiments, the antibody, or antigen binding fragment thereof, comprises a heavy chain variable domain (VH) sequence selected from an amino acid sequence set forth in any one of SEQ ID NOs: 419-488 and 818-824.

In some embodiments, the antibody, or antigen binding fragment thereof, comprises a light chain variable domain (VL) sequence selected from an amino acid sequence set forth in any one of SEQ ID NOs: 489-544 and 825-831.

In some embodiments, the antibody, or antigen binding fragment thereof, comprises a VH sequence selected from an amino acid sequence set forth in any one of SEQ ID NOs: 419-488 and 818-824, and a VL sequence selected from an amino acid sequence set forth in any one of SEQ ID NOs: 489-544 and 825-831.

In certain aspects, described herein is an isolated antibody, or antigen binding fragment thereof, that binds OX40L, wherein the antibody, or antigen binding fragment thereof, comprises a VH sequence selected from an amino acid sequence set forth in any one of SEQ ID NOs: 419-488 and 818-824, and a VL sequence selected from an amino acid sequence set forth in any one of SEQ ID NOs: 489-544 and 825-831.

In certain embodiments, the antibody, or antigen binding fragment thereof, comprises a VH sequence set forth in SEQ ID NO: 429 and a VL sequence set forth in SEQ ID NO: 497.

In certain embodiments, the antibody, or antigen binding fragment thereof, comprises a VH sequence set forth in SEQ ID NO: 429 and a VL sequence set forth in SEQ ID NO: 498.

In certain embodiments, the antibody, or antigen binding fragment thereof, comprises a VH sequence set forth in SEQ ID NO: 430 and a VL sequence set forth in SEQ ID NO: 499.

In certain embodiments, the antibody, or antigen binding fragment thereof, comprises a VH sequence set forth in SEQ ID NO: 431 and a VL sequence set forth in SEQ ID NO: 500.

In certain embodiments, the antibody, or antigen binding fragment thereof, comprises a VH sequence set forth in SEQ ID NO: 432 and a VL sequence set forth in SEQ ID NO: 501.

In certain embodiments, the antibody, or antigen binding fragment thereof, comprises a VH sequence set forth in SEQ ID NO: 433 and a VL sequence set forth in SEQ ID NO: 502.

In certain embodiments, the antibody, or antigen binding fragment thereof, comprises a VH sequence set forth in SEQ ID NO: 434 and a VL sequence set forth in SEQ ID NO: 502.

In certain embodiments, the antibody, or antigen binding fragment thereof, comprises a VH sequence set forth in SEQ ID NO: 435 and a VL sequence set forth in SEQ ID NO: 502.

In certain embodiments, the antibody, or antigen binding fragment thereof, comprises a VH sequence set forth in SEQ ID NO: 436 and a VL sequence set forth in SEQ ID NO: 502.

In certain embodiments, the antibody, or antigen binding fragment thereof, comprises a VH sequence set forth in SEQ ID NO: 437 and a VL sequence set forth in SEQ ID NO: 502.

In certain embodiments, the antibody, or antigen binding fragment thereof, comprises a VH sequence set forth in SEQ ID NO: 438 and a VL sequence set forth in SEQ ID NO: 503.

In certain embodiments, the antibody, or antigen binding fragment thereof, comprises a VH sequence set forth in SEQ ID NO: 439 and a VL sequence set forth in SEQ ID NO: 502.

In certain embodiments, the antibody, or antigen binding fragment thereof, comprises a VH sequence set forth in SEQ ID NO: 440 and a VL sequence set forth in SEQ ID NO: 504.

In certain embodiments, the antibody, or antigen binding fragment thereof, comprises a VH sequence set forth in SEQ ID NO: 441 and a VL sequence set forth in SEQ ID NO: 505.

In certain embodiments, the antibody, or antigen binding fragment thereof, comprises a VH sequence set forth in SEQ ID NO: 442 and a VL sequence set forth in SEQ ID NO: 506.

In certain embodiments, the antibody, or antigen binding fragment thereof, comprises a VH sequence set forth in SEQ ID NO: 443 and a VL sequence set forth in SEQ ID NO: 507.

In certain embodiments, the antibody, or antigen binding fragment thereof, comprises a VH sequence set forth in SEQ ID NO: 443 and a VL sequence set forth in SEQ ID NO: 508.

In certain embodiments, the antibody, or antigen binding fragment thereof, comprises a VH sequence set forth in SEQ ID NO: 444 and a VL sequence set forth in SEQ ID NO: 506.

In certain embodiments, the antibody, or antigen binding fragment thereof, comprises a VH sequence set forth in SEQ ID NO: 445 and a VL sequence set forth in SEQ ID NO: 509.

In certain embodiments, the antibody, or antigen binding fragment thereof, comprises a VH sequence set forth in SEQ ID NO: 446 and a VL sequence set forth in SEQ ID NO: 510.

In certain embodiments, the antibody, or antigen binding fragment thereof, comprises a VH sequence set forth in SEQ ID NO: 447 and a VL sequence set forth in SEQ ID NO: 506.

In certain embodiments, the antibody, or antigen binding fragment thereof, comprises a VH sequence set forth in SEQ ID NO: 445 and a VL sequence set forth in SEQ ID NO: 511.

In certain embodiments, the antibody, or antigen binding fragment thereof, comprises a VH sequence set forth in SEQ ID NO: 448 and a VL sequence set forth in SEQ ID NO: 506.

In certain embodiments, the antibody, or antigen binding fragment thereof, comprises a VH sequence set forth in SEQ ID NO: 449 and a VL sequence set forth in SEQ ID NO: 502.

In certain embodiments, the antibody, or antigen binding fragment thereof, comprises a VH sequence set forth in SEQ ID NO: 449 and a VL sequence set forth in SEQ ID NO: 506.

In certain embodiments, the antibody, or antigen binding fragment thereof, comprises a VH sequence set forth in SEQ ID NO: 450 and a VL sequence set forth in SEQ ID NO: 506.

In certain embodiments, the antibody, or antigen binding fragment thereof, comprises a VH sequence set forth in SEQ ID NO: 445 and a VL sequence set forth in SEQ ID NO: 512.

In certain embodiments, the antibody, or antigen binding fragment thereof, comprises a VH sequence set forth in SEQ ID NO: 449 and a VL sequence set forth in SEQ ID NO: 513.

In certain embodiments, the antibody, or antigen binding fragment thereof, comprises a VH sequence set forth in SEQ ID NO: 445 and a VL sequence set forth in SEQ ID NO: 514.

In certain embodiments, the antibody, or antigen binding fragment thereof, comprises a VH sequence set forth in SEQ ID NO: 445 and a VL sequence set forth in SEQ ID NO: 515.

In certain embodiments, the antibody, or antigen binding fragment thereof, comprises a VH sequence set forth in SEQ ID NO: 451 and a VL sequence set forth in SEQ ID NO: 514.

In certain embodiments, the antibody, or antigen binding fragment thereof, comprises a VH sequence set forth in SEQ ID NO: 451 and a VL sequence set forth in SEQ ID NO: 506.

In certain embodiments, the antibody, or antigen binding fragment thereof, comprises a VH sequence set forth in SEQ ID NO: 452 and a VL sequence set forth in SEQ ID NO: 506.

In certain embodiments, the antibody, or antigen binding fragment thereof, comprises a VH sequence set forth in SEQ ID NO: 438 and a VL sequence set forth in SEQ ID NO: 506.

In certain embodiments, the antibody, or antigen binding fragment thereof, comprises a VH sequence set forth in SEQ ID NO: 453 and a VL sequence set forth in SEQ ID NO: 512.

In certain embodiments, the antibody, or antigen binding fragment thereof, comprises a VH sequence set forth in SEQ ID NO: 454 and a VL sequence set forth in SEQ ID NO: 490.

In certain embodiments, the antibody, or antigen binding fragment thereof, comprises a VH sequence set forth in SEQ ID NO: 455 and a VL sequence set forth in SEQ ID NO: 516.

In certain embodiments, the antibody, or antigen binding fragment thereof, comprises a VH sequence set forth in SEQ ID NO: 456 and a VL sequence set forth in SEQ ID NO: 517.

In certain embodiments, the antibody, or antigen binding fragment thereof, comprises a VH sequence set forth in SEQ ID NO: 457 and a VL sequence set forth in SEQ ID NO: 518.

In certain embodiments, the antibody, or antigen binding fragment thereof, comprises a VH sequence set forth in SEQ ID NO: 458 and a VL sequence set forth in SEQ ID NO: 492.

In certain embodiments, the antibody, or antigen binding fragment thereof, comprises a VH sequence set forth in SEQ ID NO: 459 and a VL sequence set forth in SEQ ID NO: 519.

In certain embodiments, the antibody, or antigen binding fragment thereof, comprises a VH sequence set forth in SEQ ID NO: 460 and a VL sequence set forth in SEQ ID NO: 520.

In certain embodiments, the antibody, or antigen binding fragment thereof, comprises a VH sequence set forth in SEQ ID NO: 461 and a VL sequence set forth in SEQ ID NO: 521.

In certain embodiments, the antibody, or antigen binding fragment thereof, comprises a VH sequence set forth in SEQ ID NO: 461 and a VL sequence set forth in SEQ ID NO: 522.

In certain embodiments, the antibody, or antigen binding fragment thereof, comprises a VH sequence set forth in SEQ ID NO: 461 and a VL sequence set forth in SEQ ID NO: 522.

In certain embodiments, the antibody, or antigen binding fragment thereof, comprises a VH sequence set forth in SEQ ID NO: 462 and a VL sequence set forth in SEQ ID NO: 523.

In certain embodiments, the antibody, or antigen binding fragment thereof, comprises a VH sequence set forth in SEQ ID NO: 463 and a VL sequence set forth in SEQ ID NO: 523.

In certain embodiments, the antibody, or antigen binding fragment thereof, comprises a VH sequence set forth in SEQ ID NO: 464 and a VL sequence set forth in SEQ ID NO: 524.

In certain embodiments, the antibody, or antigen binding fragment thereof, comprises a VH sequence set forth in SEQ ID NO: 465 and a VL sequence set forth in SEQ ID NO: 525.

In certain embodiments, the antibody, or antigen binding fragment thereof, comprises a VH sequence set forth in SEQ ID NO: 466 and a VL sequence set forth in SEQ ID NO: 526.

In certain embodiments, the antibody, or antigen binding fragment thereof, comprises a VH sequence set forth in SEQ ID NO: 467 and a VL sequence set forth in SEQ ID NO: 527.

In certain embodiments, the antibody, or antigen binding fragment thereof, comprises a VH sequence set forth in SEQ ID NO: 468 and a VL sequence set forth in SEQ ID NO: 528.

In certain embodiments, the antibody, or antigen binding fragment thereof, comprises a VH sequence set forth in SEQ ID NO: 469 and a VL sequence set forth in SEQ ID NO: 529.

In certain embodiments, the antibody, or antigen binding fragment thereof, comprises a VH sequence set forth in SEQ ID NO: 470 and a VL sequence set forth in SEQ ID NO: 528.

In certain embodiments, the antibody, or antigen binding fragment thereof, comprises a VH sequence set forth in SEQ ID NO: 471 and a VL sequence set forth in SEQ ID NO: 530.

In certain embodiments, the antibody, or antigen binding fragment thereof, comprises a VH sequence set forth in SEQ ID NO: 472 and a VL sequence set forth in SEQ ID NO: 531.

In certain embodiments, the antibody, or antigen binding fragment thereof, comprises a VH sequence set forth in SEQ ID NO: 473 and a VL sequence set forth in SEQ ID NO: 532

In certain embodiments, the antibody, or antigen binding fragment thereof, comprises a VH sequence set forth in SEQ ID NO: 474 and a VL sequence set forth in SEQ ID NO: 533.

In certain embodiments, the antibody, or antigen binding fragment thereof, comprises a VH sequence set forth in SEQ ID NO: 475 and a VL sequence set forth in SEQ ID NO: 534.

In certain embodiments, the antibody, or antigen binding fragment thereof, comprises a VH sequence set forth in SEQ ID NO: 476 and a VL sequence set forth in SEQ ID NO: 535.

In certain embodiments, the antibody, or antigen binding fragment thereof, comprises a VH sequence set forth in SEQ ID NO: 477 and a VL sequence set forth in SEQ ID NO: 536.

In certain embodiments, the antibody, or antigen binding fragment thereof, comprises a VH sequence set forth in SEQ ID NO: 478 and a VL sequence set forth in SEQ ID NO: 536.

In certain embodiments, the antibody, or antigen binding fragment thereof, comprises a VH sequence set forth in SEQ ID NO: 479 and a VL sequence set forth in SEQ ID NO: 537.

In certain embodiments, the antibody, or antigen binding fragment thereof, comprises a VH sequence set forth in SEQ ID NO: 480 and a VL sequence set forth in SEQ ID NO: 538.

In certain embodiments, the antibody, or antigen binding fragment thereof, comprises a VH sequence set forth in SEQ ID NO: 481 and a VL sequence set forth in SEQ ID NO: 539.

In certain embodiments, the antibody, or antigen binding fragment thereof, comprises a VH sequence set forth in SEQ ID NO: 482 and a VL sequence set forth in SEQ ID NO: 539.

In certain embodiments, the antibody, or antigen binding fragment thereof, comprises a VH sequence set forth in SEQ ID NO: 483 and a VL sequence set forth in SEQ ID NO: 539.

In certain embodiments, the antibody, or antigen binding fragment thereof, comprises a VH sequence set forth in SEQ ID NO: 484 and a VL sequence set forth in SEQ ID NO: 540.

In certain embodiments, the antibody, or antigen binding fragment thereof, comprises a VH sequence set forth in SEQ ID NO: 485 and a VL sequence set forth in SEQ ID NO: 541.

In certain embodiments, the antibody, or antigen binding fragment thereof, comprises a VH sequence set forth in SEQ ID NO: 486 and a VL sequence set forth in SEQ ID NO: 542.

In certain embodiments, the antibody, or antigen binding fragment thereof, comprises a VH sequence set forth in SEQ ID NO: 487 and a VL sequence set forth in SEQ ID NO: 543.

In certain embodiments, the antibody, or antigen binding fragment thereof, comprises a VH sequence set forth in SEQ ID NO: 488 and a VL sequence set forth in SEQ ID NO: 544.

In certain embodiments, the antibody, or antigen binding fragment thereof, comprises a VH sequence set forth in SEQ ID NO: 818 and a VL sequence set forth in SEQ ID NO: 825.

In certain embodiments, the antibody, or antigen binding fragment thereof, comprises a VH sequence set forth in SEQ ID NO: 819 and a VL sequence set forth in SEQ ID NO: 826.

In certain embodiments, the antibody, or antigen binding fragment thereof, comprises a VH sequence set forth in SEQ ID NO: 820 and a VL sequence set forth in SEQ ID NO: 827.

In certain embodiments, the antibody, or antigen binding fragment thereof, comprises a VH sequence set forth in SEQ ID NO: 821 and a VL sequence set forth in SEQ ID NO: 828.

In certain embodiments, the antibody, or antigen binding fragment thereof, comprises a VH sequence set forth in SEQ ID NO: 822 and a VL sequence set forth in SEQ ID NO: 829.

In certain embodiments, the antibody, or antigen binding fragment thereof, comprises a VH sequence set forth in SEQ ID NO: 823 and a VL sequence set forth in SEQ ID NO: 830.

In certain embodiments, the antibody, or antigen binding fragment thereof, comprises a VH sequence set forth in SEQ ID NO: 824 and a VL sequence set forth in SEQ ID NO: 831.

In some embodiments, the antibody, or antigen binding fragment thereof, is a humanized, human, or chimeric antibody.

In some embodiments, the antibody, or antigen binding fragment thereof, is a humanized antibody.

In some embodiments, the antibody, or antigen binding fragment thereof, comprises a heavy chain human constant region of a class selected from IgG, IgA, IgD, IgE, and IgM.

In some embodiments, the antibody, or antigen binding fragment thereof, comprises a human Fc region comprising a human heavy chain constant region of the class IgG and a subclass selected from IgG1, IgG2, IgG3, and IgG4.

In some embodiments, the human Fc region comprises a human IgG1 Fc region.

In some embodiments, the human Fc region comprises a human IgG4 Fc region.

In some embodiments, the human Fc region comprises a human IgG2 Fc region.

In some embodiments, the antibody, or antigen binding fragment thereof, comprises a heavy chain comprising a constant heavy chain sequence selected from an amino acid sequence set forth in any one of SEQ ID NOs: 637-737 and 913-1012, or an amino acid sequence having at least 90%, at least 91%, at least 92%, at least 93%, at least 94%, at least 95%, at least 96%, at least 97%, at least 98%, or at least 99% sequence identity thereto. In some embodiments, the heavy chain comprises a constant heavy chain sequence selected from an amino acid sequence set forth in SEQ ID NO: 651 or SEQ ID NO: 927, or an amino acid sequence having at least 90%, at least 91%, at least 92%, at least 93%, at least 94%, at least 95%, at least 96%, at least 97%, at least 98%, or at least 99% sequence identity thereto. In some embodiments, the heavy chain comprises a constant heavy chain sequence selected from an amino acid sequence set forth in SEQ ID NO: 651, or an amino acid sequence having at least 90%, at least 91%, at least 92%, at least 93%, at least 94%, at least 95%, at least 96%, at least 97%, at least 98%, or at least 99% sequence identity thereto. In some embodiments, the heavy chain comprises a constant heavy chain sequence selected from an amino acid sequence set forth in SEQ ID NO: 927, or an amino acid sequence having at least 90%, at least 91%, at least 92%, at least 93%, at least 94%, at least 95%, at least 96%, at least 97%, at least 98%, or at least 99% sequence identity thereto.

In some embodiments, the antibody, or antigen binding fragment thereof, comprises a light chain comprising a constant light chain sequence set forth in SEQ ID NO: 738, or an amino acid sequence having at least 90%, at least 91%, at least 92%, at least 93%, at least 94%, at least 95%, at least 96%, at least 97%, at least 98%, or at least 99% sequence identity thereto.

In some embodiments, the heavy chain comprises a constant heavy chain sequence selected from an amino acid sequence set forth in SEQ ID NO: 651 or SEQ ID NO: 927, or an amino acid sequence having at least 90%, at least 91%, at least 92%, at least 93%, at least 94%, at least 95%, at least 96%, at least 97%, at least 98%, or at least 99% sequence identity thereto and the light chain comprises a constant light chain sequence set forth in SEQ ID NO: 738, or an amino acid sequence having at least 90%, at least 91%, at least 92%, at least 93%, at least 94%, at least 95%, at least 96%, at least 97%, at least 98%, or at least 99% sequence identity thereto. In some embodiments, the heavy chain comprises a constant heavy chain sequence selected from an amino acid sequence set forth in SEQ ID NO: 651, or an amino acid sequence having at least 90%, at least 91%, at least 92%, at least 93%, at least 94%, at least 95%, at least 96%, at least 97%, at least 98%, or at least 99% sequence identity thereto and the light chain comprises a constant light chain sequence set forth in SEQ ID NO: 738, or an amino acid sequence having at least 90%, at least 91%, at least 92%, at least 93%, at least 94%, at least 95%, at least 96%, at least 97%, at least 98%, or at least 99% sequence identity thereto. In some embodiments, the heavy chain comprises a constant heavy chain sequence selected from an amino acid sequence set forth in SEQ ID NO: 927, or an amino acid sequence having at least 90%, at least 91%, at least 92%, at least 93%, at least 94%, at least 95%, at least 96%, at least 97%, at least 98%, or at least 99% sequence identity thereto and the light chain comprises a constant light chain sequence set forth in SEQ ID NO: 738, or an amino acid sequence having at least 90%, at least 91%, at least 92%, at least 93%, at least 94%, at least 95%, at least 96%, at least 97%, at least 98%, or at least 99% sequence identity thereto.

In some embodiments, the antibody, or antigen binding fragment thereof, comprises an Fc region comprising one or more amino acid substitutions, wherein the one or more amino acid substitutions result in an increase in one or more of antibody half-life, ADCC activity, ADCP activity, and/or CDC activity compared with the Fc region without the one or more amino acid substitutions.

In some embodiments, the antibody, or antigen binding fragment thereof, comprises an Fc region comprising one or more amino acid substitutions, wherein the one or more amino acid substitutions result in a decrease in one or more of ADCC activity, ADCP activity, and/or CDC activity compared with the Fc region without the one or more amino acid substitutions.

In some embodiments, the one or more amino acid substitutions results in increased antibody half-life compared to an antibody comprising a wild-type Fc region.

In some embodiments, the one or more amino acid substitutions is selected from the group consisting of S228P, M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W; optionally, wherein the one or more amino acid substitutions is a combination of amino acid substitutions selected from the group consisting of M428L/N434S (LS), M252Y/S254T/T256E (YTE), T250Q/M428L, T307A/E380A/N434A, T256D/T307Q (DQ), T256D/T307W (DW), M252Y/T256D (YD), T307Q/Q311V/A378V (QVV), T256D/H285D/T307R/Q311V/A378V (DDRVV), L309D/Q311H/N434S (DHS), S228P/L235E (SPLE), L234A/L235A (LALA), M428L/N434A (LA), L235A/G237A (LAGA), L234A/L235A/G237A (LALAGA), L234A/L235A/P329G (LALAPG), D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/N434A, D265A/N434A, LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV. In some embodiments, the one or more amino acid substitutions is selected from the group consisting of LS, YTE, T250Q/M428L, T307A/E380A/N434A, DQ, DW, YD, QVV, DHS, LA, D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, N297A/QVV, D265A/QVV, LALA/QVV, LAGA/QVV, LALAGA/QVV, and LALAPG/QVV.

Although the EU numbering system is typically used to identify the positions of the various Fc mutations described herein, direct numbering can also be used. For example, in certain embodiments, an antibody described herein comprises an Fc region with YTE mutations at positions 253/255/257, respectively. In certain embodiments, an antibody described herein comprises an Fc region with LALA mutations at positions 235/236, respectively. In certain embodiments, an antibody described herein comprises an Fc region (e.g., an IgG1 Fc region) with YTE mutations at positions 253/255/257 and with LALA mutations at positions 235/236. In certain embodiments, the OX40L antibody described herein comprises the VH and VL of Construct 114 and an Fc region (e.g., an IgG1 Fc region) comprising YTE mutations at positions 253/255/257, respectively and with LALA mutations at positions 235/236, respectively.

In certain embodiments, the antibody, or antigen binding fragment thereof, comprises a heavy chain comprising a heavy chain constant region having a means for extending the half-life of the isolated antibody.

In certain embodiments, the antibody, or antigen binding fragment thereof, comprises a VH sequence set forth in SEQ ID NO: 419, a VL sequence set forth in SEQ ID NO: 489; and a human IgG1 Fc region comprising LALA and YTE mutations. In some embodiments, the antibody comprises a VH domain comprising the amino acid sequence set forth in SEQ ID NO: 419, a VL domain comprising the amino acid sequence set forth in SEQ ID NO: 489, a constant heavy chain sequence set forth in SEQ ID NO: 927 and/or a constant heavy chain sequence set forth in SEQ ID NO: 651, and a constant light chain sequence set forth in SEQ ID NO: 738. In some embodiments, the isolated antibody comprises a VH domain comprising the amino acid sequence set forth in SEQ ID NO: 419, a VL domain comprising the amino acid sequence set forth in SEQ ID NO: 489, a constant heavy chain sequence set forth in SEQ ID NO: 927, and a constant light chain sequence set forth in SEQ ID NO: 738. In some embodiments, the isolated antibody comprises a VH domain comprising the amino acid sequence set forth in SEQ ID NO: 419, a VL domain comprising the amino acid sequence set forth in SEQ ID NO: 489, a constant heavy chain sequence set forth in SEQ ID NO: 651, and a constant light chain sequence set forth in SEQ ID NO: 738. A C-terminal lysine is present in SEQ ID NO: 927, which may be cleaved off during manufacture or after administration, resulting in the sequence of SEQ ID NO: 651. Accordingly, a composition comprising an antibody that binds OX40L comprising the constant heavy chain sequence of SEQ ID NO: 927 that is administered to a subject may comprise antibodies having the constant heavy chain sequence set forth in SEQ ID NO: 927 (e.g., an antibody comprising the heavy chain sequence of SEQ ID NO: 1013) or SEQ ID NO: 651 (e.g., an antibody comprising the heavy chain sequence of SEQ ID NO: 1014), or a mixture thereof (e.g., a mixture of antibodies containing either a constant heavy chain sequence of SEQ ID NO: 927 or a constant heavy chain sequence of SEQ ID NO: 651 and/or antibodies containing both constant heavy chain sequences (e.g., in an antibody containing two constant heavy chain sequences)).

In some embodiments, the isolated antibody comprises a heavy chain sequence set forth in SEQ ID NO: 1013 and/or SEQ ID NO: 1014 and a light chain sequence set forth in SEQ ID NO: 1015. In some embodiments, the isolated antibody comprises a heavy chain sequence set forth in SEQ ID NO: 1013 and a light chain sequence set forth in SEQ ID NO: 1015. The C-terminal lysine in SEQ ID NO: 1013 may not be present if cleavage occurs during manufacture or after administration (which would result in the heavy chain sequence of SEQ ID NO: 1014).

In some embodiments, the Fc region binds to Neonatal Fc receptor (FcRn).

In some embodiments, the Fc region binds an FcRn with higher affinity at pH 6.0 compared to an antibody comprising a wild-type Fc region.

In some embodiments, the Fc region binds to FcRn with a KD of <1×10-7 M at pH 6.0.

In some embodiments, the antibody, or antigen binding fragment thereof, is a monoclonal antibody.

In some embodiments, the antibody, or antigen binding fragment thereof, binds an OX40L sequence set forth in SEQ ID NO: 739.

In some embodiments, the antibody, or antigen binding fragment thereof, binds to an OX40L sequence set forth in SEQ ID NO: 739 with a KD of less than or equal to about 1, 2, 3, 4, 5, 6, 7, 8, or 9×10-⁹ M, as measured by surface plasmon resonance (SPR).

In some embodiments, the antibody, or antigen binding fragment thereof, binds to an OX40L sequence set forth in SEQ ID NO: 739 with a KD of less than or equal to about 1×10⁻¹⁰ M, as measured by surface plasmon resonance (SPR).

In some embodiments, the antibody, or antigen binding fragment thereof, binds to human OX40L with a KD of less than or equal to about 1×10⁻⁹ M, as measured by surface plasmon resonance (SPR).

In some embodiments, the antibody, or antigen binding fragment thereof, exhibits a melting temperature greater than 68° C. as measured by Differential Scanning Fluorometry (DSF).

In some embodiments, the antibody, or antigen binding fragment thereof, exhibits a melting temperature greater than 75° C. as measured by Differential Scanning Fluorometry (DSF).

In some embodiments, the antibody, or antigen binding fragment thereof, exhibits an aggregation temperature equal to or greater than 71.2° C. as measured by Differential Scanning Fluorometry (DSF).

In some embodiments, the antibody, or antigen binding fragment thereof, has a retention time of 15.2 minutes or less as measured by hydrophobic interaction chromatography.

In some embodiments, the antibody, or antigen binding fragment thereof, can inhibit OX40L dependent IFNγ release, for example, in a human cell. In some embodiments, the antibody, or antigen binding fragment thereof, can inhibit OX40L dependent IFNγ release, for example, in a human.

In certain embodiments, the antibody, or antigen binding fragment thereof, has an increased capacity to bind cell surface OX40L as compared to amlitelimab.

In certain embodiments, the isolated antibody, or antigen binding fragment thereof, is used in the treatment of an inflammatory disorder or disease. In certain embodiments, the isolated antibody, or antigen binding fragment thereof, is used in the treatment of atopic dermatitis. In certain embodiments, the isolated antibody, or antigen binding fragment thereof, is used in the treatment of idiopathic pulmonary fibrosis. In certain embodiments, the isolated antibody, or antigen binding fragment thereof, is used in the treatment of alopecia areata. In certain embodiments, the isolated antibody, or antigen binding fragment thereof, is used in the treatment of chronic sinusitis with nasal polyps. In certain embodiments, the isolated antibody, or antigen binding fragment thereof, is used in the treatment of Chronic Rhinosinusitis without Nasal Polyps (CRSsNP). In certain embodiments, the isolated antibody, or antigen binding fragment thereof, is used in the treatment of eosinophilic esophagitis (EoE). In certain embodiments, the isolated antibody, or antigen binding fragment thereof, is used in the treatment of Eosinophilic gastrointestinal disorder or disease (EGID) selected from the group consisting of Eosinophilic Gastritis (EoG), Eosinophilic Enteritis (EoN), Eosinophilic Colitis (EoC), and Eosinophilic Gastroenteritis (EGE). In certain embodiments, the isolated antibody, or antigen binding fragment thereof, is used in the treatment of Churg-Strauss syndrome/Eosinophilic granulomatosis with polyangiitis (EGPA). In certain embodiments, the isolated antibody, or antigen binding fragment thereof, is used in the treatment of Prurigo Nodularis (PN). In certain embodiments, the isolated antibody, or antigen binding fragment thereof, is used in the treatment of Chronic Spontaneous Urticaria (CSU). In certain embodiments, the isolated antibody, or antigen binding fragment thereof, is used in the treatment of Chronic Pruritis of Unknown Origin (CPUO). In certain embodiments, the isolated antibody, or antigen binding fragment thereof, is used in the treatment of Bullous Pemphigoid (BP). In certain embodiments, the isolated antibody, or antigen binding fragment thereof, is used in the treatment of Cold Inducible Urticaria (ColdU). In certain embodiments, the isolated antibody, or antigen binding fragment thereof, is used in the treatment of Allergic Fungal Rhinosinusitis (AFRS). In certain embodiments, the isolated antibody, or antigen binding fragment thereof, is used in the treatment of Allergic Bronchopulmonary Aspergillosis (ABPA). In certain embodiments, the isolated antibody, or antigen binding fragment thereof, is used in the treatment of Chronic Obstructive Pulmonary Disease (COPD). In certain embodiments, the isolated antibody, or antigen binding fragment thereof, is used in the treatment of inflammatory bowel disease, such as Crohn's disease or ulcerative colitis. In certain embodiments, the isolated antibody, or antigen binding fragment thereof, is used in the treatment of psoriasis. In certain embodiments, the isolated antibody, or antigen binding fragment thereof, is used in the treatment of lupus. In certain embodiments, the isolated antibody, or antigen binding fragment thereof, is used in the treatment of rheumatoid arthritis. In certain embodiments, the isolated antibody, or antigen binding fragment thereof, is used in the treatment of hidradenitis suppurativa. In certain embodiments, the isolated antibody, or antigen binding fragment thereof, is used in the treatment of celiac disease. In certain embodiments, the isolated antibody, or antigen binding fragment thereof, is used in the treatment of systemic sclerosis. In certain embodiments, the isolated antibody, or antigen binding fragment thereof, is used in the treatment of allergic rhinitis. In certain embodiments, the isolated antibody, or antigen binding fragment thereof, is used in the treatment of eosinophilic fasciitis. In certain embodiments, the isolated antibody, or antigen binding fragment thereof, is used in the treatment of scleromyxedema. In certain embodiments, the isolated antibody, or antigen binding fragment thereof, is used in the treatment of scleredema. In certain embodiments, the isolated antibody, or antigen binding fragment thereof, is used in the treatment of nephrogenic systemic fibrosis.

In certain aspects, disclosed herein is an isolated polynucleotide or set of polynucleotides encoding any one of the antibodies disclosed herein, a VH thereof, a VL thereof, a light chain thereof, a heavy chain thereof, or an antigen-binding portion thereof, and optionally, wherein the polynucleotide or set of polynucleotides comprises cDNA.

In certain aspects, disclosed herein is a vector or set of vectors comprising the polynucleotide or set of polynucleotides discloses herein.

In certain aspects, disclosed herein is a host cell comprising the polynucleotide or set of polynucleotides or the vector or set of vectors disclosed herein.

In certain aspects, disclosed herein is a method of producing an antibody, or antigen binding fragment thereof, the method comprising expressing the antibody, or antigen binding fragment thereof, with the host cell disclosed herein and isolating the expressed antibody.

In certain aspects, disclosed herein is a pharmaceutical composition comprising the antibody, or antigen binding fragment thereof, of any one of the embodiments disclosed herein and a pharmaceutically acceptable excipient. In some embodiments, the pharmaceutical composition further comprises a hyaluronidase or a variant thereof.

In certain aspects, disclosed herein is a kit comprising the antibody, or antigen binding fragment thereof, of any one of the embodiments disclosed herein or a pharmaceutical composition disclosed herein and instructions for use. In some embodiments, the kit further comprises a hyaluronidase or a variant thereof.

In certain aspects, described herein is a method for treating an inflammatory disorder or disease in a mammalian subject in need thereof, the method comprising administering to the mammalian subject a therapeutically effective amount an antibody, or antigen binding fragment thereof, described herein or a pharmaceutical composition described herein. In certain embodiments of the methods described herein, the inflammatory disorder or disease is atopic dermatitis. In certain embodiments, the inflammatory disorder or disease is asthma. In certain embodiments, the inflammatory disorder or disease is idiopathic pulmonary fibrosis. In certain embodiments, the inflammatory disorder or disease is alopecia areata. In certain embodiments, the inflammatory disorder or disease is chronic sinusitis with nasal polyps. In certain embodiments, the inflammatory disorder or disease is Chronic Rhinosinusitis without Nasal Polyps (CRSsNP). In certain embodiments, the inflammatory disorder or disease is eosinophilic esophagitis (EoE). In certain embodiments, the inflammatory disorder or disease is an Eosinophilic gastrointestinal disorder or disease (EGID), such as Eosinophilic Gastritis (EoG), Eosinophilic Enteritis (EoN), Eosinophilic Colitis (EoC), or Eosinophilic Gastroenteritis (EGE). In certain embodiments, the inflammatory disorder or disease is Churg-Strauss syndrome/Eosinophilic granulomatosis with polyangiitis (EGPA). In certain embodiments, the inflammatory disorder or disease is Prurigo Nodularis (PN). In certain embodiments, the inflammatory disorder or disease is Chronic Spontaneous Urticaria (CSU). In certain embodiments, the inflammatory disorder or disease is Chronic Pruritis of Unknown Origin (CPUO). In certain embodiments, the inflammatory disorder or disease is Bullous Pemphigoid (BP). In certain embodiments, the inflammatory disorder or disease is Cold Inducible Urticaria (ColdU). In certain embodiments, the inflammatory disorder or disease is Allergic Fungal Rhinosinusitis (AFRS). In certain embodiments, the inflammatory disorder or disease is Allergic Bronchopulmonary Aspergillosis (ABPA). In certain embodiments, the inflammatory disorder or disease is Chronic Obstructive Pulmonary Disease (COPD). In certain embodiments, the inflammatory disorder or disease is inflammatory bowel disease, such as Crohn's disease or ulcerative colitis. In certain embodiments, the inflammatory disorder or disease is psoriasis. In certain embodiments, the inflammatory disorder or disease is lupus. In certain embodiments, the inflammatory disorder or disease is rheumatoid arthritis. In certain embodiments, the inflammatory disorder or disease is celiac disease. In certain embodiments, the inflammatory disorder or disease is hidradenitis suppurativa. In certain embodiments, the inflammatory disorder or disease is systemic sclerosis. In certain embodiments, the inflammatory disorder or disease is allergic rhinitis. In certain embodiments, the inflammatory disorder or disease is eosinophilic fasciitis. In certain embodiments, the inflammatory disorder or disease is scleromyxedema. In certain embodiments, the inflammatory disorder or disease is scleredema. In certain embodiments, the inflammatory disorder or disease is nephrogenic systemic fibrosis.

In certain embodiments of the methods described herein, the mammalian subject is a human.

In certain aspects, disclosed herein is a method for treating a pathology associated with elevated levels of OX40L in a mammalian subject in need thereof, the method comprising administering to the mammalian subject a therapeutically effective amount the antibody, or antigen binding fragment thereof, of any one of the embodiments disclosed herein or a pharmaceutical composition disclosed herein. In certain embodiments of the methods described herein, the mammalian subject is a human.

In certain aspects, disclosed herein is a method of reducing biological activity of OX40L in a mammalian subject in need thereof, the method comprising administering to the mammalian subject a therapeutically effective amount the antibody, or antigen binding fragment thereof, of any one of the embodiments disclosed herein or a pharmaceutical composition disclosed herein. In certain embodiments of the methods described herein, the mammalian subject is a human.

In certain aspects, disclosed herein is a method of inhibiting the TH2 type (type 2) allergic response in a mammalian subject in need thereof, the method comprising administering to the mammalian subject a therapeutically effective amount the antibody, or antigen binding fragment thereof, of any one of the embodiments disclosed herein or a pharmaceutical composition disclosed herein. In certain embodiments of the methods described herein, the mammalian subject is a human.

In certain aspects, disclosed herein is a method of preventing an inflammatory disorder or disease in a mammalian subject in need thereof, the method comprising administering to the mammalian subject a therapeutically effective amount the antibody, or antigen binding fragment thereof, of any one of the embodiments disclosed herein or a pharmaceutical composition disclosed herein. In certain embodiments of the methods described herein, the mammalian subject is a human.

In some embodiments of any of the foregoing methods, the method further includes administering a hyaluronidase or a variant thereof. In some embodiments of any of the foregoing methods, the pharmaceutical composition comprises an antibody, or antigen binding fragment thereof, described herein and a hyaluronidase or a variant thereof.

In certain aspects, described herein are compositions comprising any of the antibodies, or antigen binding fragments thereof, that bind OX40L described herein and a hyaluronidase or variant thereof.

In certain aspects, described herein are a combination of any one of the antibodies, or antigen binding fragments thereof, that bind OX40L described herein and a hyaluronidase or variant thereof.

In certain aspects, described herein are methods of treating an inflammatory disorder or disease in a human patient comprising co-administering to the patient any one of the antibodies, or antigen binding fragments thereof, that bind OX40L described herein and a hyaluronidase or variant thereof.

In certain embodiments, the antibody, or antigen binding fragment thereof, is a humanized, human, or chimeric antibody. In certain embodiments, the antibody, or antigen binding fragment thereof, is a monoclonal antibody. In certain embodiments, the antibody, or antigen binding fragment thereof, comprises a heavy chain human constant region of a class selected from IgG, IgA, IgD, IgE, and IgM. In certain embodiments, the antibody, or antigen binding fragment thereof, comprises a human Fc region comprising a human heavy chain constant region of the class IgG and a subclass selected from IgG1, IgG2, IgG3, and IgG4. In certain embodiments, the human Fc region comprises a human IgG1 Fc region.

In certain embodiments the human Fc region comprises a human IgG1 Fc region with LALA mutations. In certain embodiments the human Fc region comprises a human IgG1 Fc region with YTE mutations. In certain embodiments the human Fc region comprises a human IgG1 Fc region with LALA and YTE mutations. In certain embodiments, when direct numbering is used these “YTE” and “LALA” mutations can be located at different amino acid position numbers. For example, in one embodiment, the human Fc region comprises a human IgG1 Fc region with LALA mutations at L235A/L236A and/or YTE mutations at M253Y/S255T/T257E.

In certain embodiments, the antibody, or antigen binding fragment thereof, that binds OX40L comprises a CDR-H1 comprising the sequence set forth in SEQ ID NO:1; a CDR-H2 comprising the sequence set forth in SEQ ID NO: 70; a CDR-H3 comprising the sequence set forth in SEQ ID NO: 175; a CDR-L1 comprising the sequence set forth in SEQ ID NO: 258; a CDR-L2 comprising the sequence set forth in SEQ ID NO: 335; and a CDR-L3 comprising the sequence set forth in SEQ ID NO: 368. In certain embodiments, the antibody, or antigen binding fragment thereof, that binds OX40L comprises a CDR-H1 comprising the sequence set forth in SEQ ID NO: 44; a CDR-H2 comprising the sequence set forth in SEQ ID NO: 910; a CDR-H3 comprising the sequence set forth in SEQ ID NO: 911; a CDR-L1 comprising the sequence set forth in SEQ ID NO: 334; a CDR-L2 comprising the sequence set forth in SEQ ID NO: 361; and a CDR-L3 comprising the sequence set forth in SEQ ID NO: 912. In certain embodiments, the antibody, or antigen binding fragment thereof, that binds OX40L comprises a VH sequence set forth in SEQ ID NO: 419 and a VL sequence set forth in SEQ ID NO: 489. In certain embodiments, the antibody, or antigen binding fragment thereof, that binds OX40L comprises a VH sequence set forth in SEQ ID NO: 832 and a VL sequence set forth in SEQ ID NO: 833.

In certain embodiments, the antibody, or antigen binding fragment thereof, comprises a human Fc region comprising a human IgG sequence selected from a sequence set forth in any one of SEQ ID NOs: 637-737 and 913-1012. In certain embodiments, the antibody, or antigen binding fragment thereof comprises a heavy chain comprising a constant heavy chain sequence selected from a sequence set forth in any one of SEQ ID NOs: 637-639, 641-737, 913-915, and 917-1012. In certain embodiments, the antibody, or antigen binding fragment thereof does not comprise a constant heavy chain sequence set forth in SEQ ID NOs: 640 and 916 (i.e., a human IgG4-SPLE constant heavy chain region). In certain embodiments, the antibody, or antigen binding fragment thereof, comprises a heavy chain comprising a constant heavy chain sequence set forth in SEQ ID NO: 927 or SEQ ID NO: 651. In certain embodiments, the antibody, or antigen binding fragment thereof, comprises a heavy chain comprising a constant heavy chain sequence set forth in SEQ ID NO: 927. In certain embodiments, the antibody, or antigen binding fragment thereof, comprises a light chain comprising a constant light chain sequence set forth in SEQ ID NO: 738.

In some embodiments, the antibody, or antigen binding fragment thereof, comprises an Fc region comprising one or more amino acid substitutions, wherein the one or more amino acid substitutions result in an increase in one or more of antibody half-life, ADCC activity, ADCP activity, or CDC activity compared with the Fc region without the one or more amino acid substitutions.

In some embodiments, the antibody, or antigen binding fragment thereof, comprises an Fc region comprising one or more amino acid substitutions, wherein the one or more amino acid substitutions result in a decrease in one or more of, ADCC activity, ADCP activity or CDC activity compared to an antibody comprising a wild-type Fc region.

In certain embodiments, the one or more amino acid substitutions is selected from the group consisting of S228P, M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W; optionally, wherein the one or more amino acid substitutions comprises a plurality of amino acid substitutions selected from the group consisting of M428L/N434S (LS), M252Y/S254T/T256E (YTE), T250Q/M428L, T307A/E380A/N434A, T256D/T307Q (DQ), T256D/T307W (DW), M252Y/T256D (YD), T307Q/Q311V/A378V (QVV), T256D/H285D/T307R/Q311V/A378V (DDRVV), L309D/Q311H/N434S (DHS), S228P/L235E (SPLE), L234A/L235A (LALA), M428L/N434A (LA), L235A/G237A (LAGA), L234A/L235A/G237A (LALAGA), L234A/L235A/P329G (LALAPG), D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/N434A, D265A/N434A, LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV. In certain embodiments, the one or more amino acid substitutions is selected from the group consisting of LS, YTE, T250Q/M428L, T307A/E380A/N434A, DQ, DW, YD, QVV, DHS, LA, D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, N297A/QVV, D265A/QVV, LALA/QVV, LAGA/QVV, LALAGA/QVV, and LALAPG/QVV.

In certain embodiments, the antibody, or antigen binding fragment thereof, comprises a human IgG1 Fc region with LALA and YTE mutations.

In certain embodiments, the antibody, or antigen binding fragment thereof, comprises a VH domain comprising the amino acid sequence set forth in SEQ ID NO: 419, a VL domain comprising the amino acid sequence set forth in SEQ ID NO: 489, and a human IgG1 Fc region comprising LALA and YTE substitutions. In certain embodiments, the antibody, or antigen binding fragment thereof, comprises a VH domain comprising the amino acid sequence set forth in SEQ ID NO: 419, a VL domain comprising the amino acid sequence set forth in SEQ ID NO: 489, a constant heavy chain sequence set forth in SEQ ID NO: 927 and/or a constant heavy chain sequence set forth in SEQ ID NO: 651, and a constant light chain sequence set forth in SEQ ID NO: 738. In certain embodiments, the antibody, or antigen binding fragment thereof, comprises a VH domain comprising the amino acid sequence set forth in SEQ ID NO: 419, a VL domain comprising the amino acid sequence set forth in SEQ ID NO: 489, a constant heavy chain sequence set forth in SEQ ID NO: 927, and a constant light chain sequence set forth in SEQ ID NO: 738. In certain embodiments, the antibody, or antigen binding fragment thereof, comprises a heavy chain sequence set forth in SEQ ID NO: 1013 and a light chain sequence set forth in SEQ ID NO: 1015. In certain embodiments, the antibody, or antigen binding fragment thereof, comprises a VH domain comprising the amino acid sequence set forth in SEQ ID NO: 419, a VL domain comprising the amino acid sequence set forth in SEQ ID NO: 489, a constant heavy chain sequence set forth in SEQ ID NO: 651, and a constant light chain sequence set forth in SEQ ID NO: 738. In certain embodiments, the antibody, or antigen binding fragment thereof, comprises a heavy chain sequence set forth in SEQ ID NO: 1014 and a light chain sequence set forth in SEQ ID NO: 1015.

In certain embodiments, the Fc region of the antibody binds to Neonatal Fc receptor (FcRn). In certain embodiments, the Fc region binds an FcRn with higher affinity at pH 6.0 compared to an antibody comprising a wild-type Fc region. In certain embodiments, the Fc region binds to FcRn with a K_{D} of <1×10⁻⁷ M at pH 6.0. In certain embodiments, the antibody, or antigen binding fragment thereof, binds an OX40L sequence set forth in SEQ ID NO: 739.

Any suitable hyaluronidase or variant thereof can be used in the compositions, combinations, and methods described herein. In certain embodiments, the hyaluronidase is a soluble hyaluronidase. In certain embodiments, the hyaluronidase is a recombinant human hyaluronidase. In certain embodiments, the recombinant human hyaluronidase comprises the amino acid sequence set forth in any one of SEQ ID NOs: 843-852 and 854-909, or a mixture thereof. In certain embodiments, the recombinant human hyaluronidase is rHuPH20 (i.e., a composition comprising one or more of SEQ ID NOs: 845-850). In certain embodiments, the rHuPH20 formulation is ENHANZE®.

In one embodiment, the recombinant human hyaluronidase includes a sequence of amino acids in any one of SEQ ID NOs: 843-852 and 854-909, or has at least 80%, 81%, 82%, 83%, 84%, 85%, 86%, 87%, 88%, 89%, 90%, 91%, 92%, 93%, 94%, 95%, 96%, 97%, 98%, or 99% sequence identity to a sequence of amino acids included in any one of SEQ ID NOs: 843-852 and 854-909 and retains hyaluronidase activity. In one embodiment, the recombinant human hyaluronidase comprises a sequence having at least 95%, 96%, 97%, 98%, or 99% to the amino acid sequence of SEQ ID NO: 843. In one embodiment, the recombinant human hyaluronidase comprises a sequence having at least 95%, 96%, 97%, 98%, or 99% to the amino acid sequence of SEQ ID NO: 845. In another embodiment, the recombinant human hyaluronidase comprises the amino acid sequence set forth in SEQ ID NO:843. In another embodiment, the recombinant human hyaluronidase consists of the amino acid sequence set forth in SEQ ID NO:843. In another embodiment, the recombinant human hyaluronidase comprises the amino acid sequence set forth in SEQ ID NO:844. In another embodiment, the recombinant human hyaluronidase consists of the amino acid sequence set forth in SEQ ID NO:844. In another embodiment, the recombinant human hyaluronidase comprises the amino acid sequence set forth in SEQ ID NO:845. In another embodiment, the recombinant human hyaluronidase consists of the amino acid sequence set forth in SEQ ID NO:845. In another embodiment, the recombinant human hyaluronidase comprises the amino acid sequence set forth in SEQ ID NO:846. In another embodiment, the recombinant human hyaluronidase consists of the amino acid sequence set forth in SEQ ID NO:846. In another embodiment, the recombinant human hyaluronidase comprises the amino acid sequence set forth in SEQ ID NO:847. In another embodiment, the recombinant human hyaluronidase consists of the amino acid sequence set forth in SEQ ID NO:847. In another embodiment, the recombinant human hyaluronidase comprises the amino acid sequence set forth in SEQ ID NO:848. In another embodiment, the recombinant human hyaluronidase consists of the amino acid sequence set forth in SEQ ID NO:848. In another embodiment, the recombinant human hyaluronidase comprises the amino acid sequence set forth in SEQ ID NO:849. In another embodiment, the recombinant human hyaluronidase consists of the amino acid sequence set forth in SEQ ID NO:849. In another embodiment, the recombinant human hyaluronidase comprises the amino acid sequence set forth in SEQ ID NO:850. In another embodiment, the recombinant human hyaluronidase consists of the amino acid sequence set forth in SEQ ID NO:850. In another embodiment, the recombinant human hyaluronidase comprises the amino acid sequence set forth in SEQ ID NO:851. In another embodiment, the recombinant human hyaluronidase consists of the amino acid sequence set forth in SEQ ID NO:851. In another embodiment, the recombinant human hyaluronidase comprises the amino acid sequence set forth in SEQ ID NO:852. In another embodiment, the recombinant human hyaluronidase consists of the amino acid sequence set forth in SEQ ID NO:852. In another embodiment, the recombinant human hyaluronidase comprises the amino acid sequence set forth in SEQ ID NO:854. In another embodiment, the recombinant human hyaluronidase consists of the amino acid sequence set forth in SEQ ID NO:854. In another embodiment, the recombinant human hyaluronidase comprises the amino acid sequence set forth in SEQ ID NO:855. In another embodiment, the recombinant human hyaluronidase consists of the amino acid sequence set forth in SEQ ID NO:855. In another embodiment, the recombinant human hyaluronidase comprises the amino acid sequence set forth in SEQ ID NO:856. In another embodiment, the recombinant human hyaluronidase consists of the amino acid sequence set forth in SEQ ID NO:856. In another embodiment, the recombinant human hyaluronidase comprises the amino acid sequence set forth in SEQ ID NO:857. In another embodiment, the recombinant human hyaluronidase consists of the amino acid sequence set forth in SEQ ID NO:857. In another embodiment, the recombinant human hyaluronidase comprises the amino acid sequence set forth in SEQ ID NO:858. In another embodiment, the recombinant human hyaluronidase consists of the amino acid sequence set forth in SEQ ID NO:858. In another embodiment, the recombinant human hyaluronidase comprises the amino acid sequence set forth in SEQ ID NO:859. In another embodiment, the recombinant human hyaluronidase consists of the amino acid sequence set forth in SEQ ID NO:859. In another embodiment, the recombinant human hyaluronidase comprises the amino acid sequence set forth in SEQ ID NO:860. In another embodiment, the recombinant human hyaluronidase consists of the amino acid sequence set forth in SEQ ID NO:860. In another embodiment, the recombinant human hyaluronidase comprises the amino acid sequence set forth in SEQ ID NO:861. In another embodiment, the recombinant human hyaluronidase consists of the amino acid sequence set forth in SEQ ID NO:861. In another embodiment, the recombinant human hyaluronidase comprises the amino acid sequence set forth in SEQ ID NO:862. In another embodiment, the recombinant human hyaluronidase consists of the amino acid sequence set forth in SEQ ID NO:862. In another embodiment, the recombinant human hyaluronidase comprises the amino acid sequence set forth in SEQ ID NO:863. In another embodiment, the recombinant human hyaluronidase consists of the amino acid sequence set forth in SEQ ID NO:863. In another embodiment, the recombinant human hyaluronidase comprises the amino acid sequence set forth in SEQ ID NO:864. In another embodiment, the recombinant human hyaluronidase consists of the amino acid sequence set forth in SEQ ID NO:864. In another embodiment, the recombinant human hyaluronidase comprises the amino acid sequence set forth in SEQ ID NO:865. In another embodiment, the recombinant human hyaluronidase consists of the amino acid sequence set forth in SEQ ID NO:865. In another embodiment, the recombinant human hyaluronidase comprises the amino acid sequence set forth in SEQ ID NO:866. In another embodiment, the recombinant human hyaluronidase consists of the amino acid sequence set forth in SEQ ID NO:866. In another embodiment, the recombinant human hyaluronidase comprises the amino acid sequence set forth in SEQ ID NO:867. In another embodiment, the recombinant human hyaluronidase consists of the amino acid sequence set forth in SEQ ID NO:867. In another embodiment, the recombinant human hyaluronidase comprises the amino acid sequence set forth in SEQ ID NO:868. In another embodiment, the recombinant human hyaluronidase consists of the amino acid sequence set forth in SEQ ID NO:868. In another embodiment, the recombinant human hyaluronidase comprises the amino acid sequence set forth in SEQ ID NO:869. In another embodiment, the recombinant human hyaluronidase consists of the amino acid sequence set forth in SEQ ID NO:869. In another embodiment, the recombinant human hyaluronidase comprises the amino acid sequence set forth in SEQ ID NO:870. In another embodiment, the recombinant human hyaluronidase consists of the amino acid sequence set forth in SEQ ID NO:870. In another embodiment, the recombinant human hyaluronidase comprises the amino acid sequence set forth in SEQ ID NO:871. In another embodiment, the recombinant human hyaluronidase consists of the amino acid sequence set forth in SEQ ID NO:871. In another embodiment, the recombinant human hyaluronidase comprises the amino acid sequence set forth in SEQ ID NO:872. In another embodiment, the recombinant human hyaluronidase consists of the amino acid sequence set forth in SEQ ID NO:872. In another embodiment, the recombinant human hyaluronidase comprises the amino acid sequence set forth in SEQ ID NO:873. In another embodiment, the recombinant human hyaluronidase consists of the amino acid sequence set forth in SEQ ID NO:873. In another embodiment, the recombinant human hyaluronidase comprises the amino acid sequence set forth in SEQ ID NO:874. In another embodiment, the recombinant human hyaluronidase consists of the amino acid sequence set forth in SEQ ID NO:874. In another embodiment, the recombinant human hyaluronidase comprises the amino acid sequence set forth in SEQ ID NO:875. In another embodiment, the recombinant human hyaluronidase consists of the amino acid sequence set forth in SEQ ID NO:875. In another embodiment, the recombinant human hyaluronidase comprises the amino acid sequence set forth in SEQ ID NO:876. In another embodiment, the recombinant human hyaluronidase consists of the amino acid sequence set forth in SEQ ID NO:876. In another embodiment, the recombinant human hyaluronidase comprises the amino acid sequence set forth in SEQ ID NO:877. In another embodiment, the recombinant human hyaluronidase consists of the amino acid sequence set forth in SEQ ID NO:877. In another embodiment, the recombinant human hyaluronidase comprises the amino acid sequence set forth in SEQ ID NO:878. In another embodiment, the recombinant human hyaluronidase consists of the amino acid sequence set forth in SEQ ID NO:878. In another embodiment, the recombinant human hyaluronidase comprises the amino acid sequence set forth in SEQ ID NO:879. In another embodiment, the recombinant human hyaluronidase consists of the amino acid sequence set forth in SEQ ID NO:879. In another embodiment, the recombinant human hyaluronidase comprises the amino acid sequence set forth in SEQ ID NO:880. In another embodiment, the recombinant human hyaluronidase consists of the amino acid sequence set forth in SEQ ID NO:880. In another embodiment, the recombinant human hyaluronidase comprises the amino acid sequence set forth in SEQ ID NO:881. In another embodiment, the recombinant human hyaluronidase consists of the amino acid sequence set forth in SEQ ID NO:881. In another embodiment, the recombinant human hyaluronidase comprises the amino acid sequence set forth in SEQ ID NO:882. In another embodiment, the recombinant human hyaluronidase consists of the amino acid sequence set forth in SEQ ID NO:882. In another embodiment, the recombinant human hyaluronidase comprises the amino acid sequence set forth in SEQ ID NO:883. In another embodiment, the recombinant human hyaluronidase consists of the amino acid sequence set forth in SEQ ID NO:883. In another embodiment, the recombinant human hyaluronidase comprises the amino acid sequence set forth in SEQ ID NO:884. In another embodiment, the recombinant human hyaluronidase consists of the amino acid sequence set forth in SEQ ID NO:884. In another embodiment, the recombinant human hyaluronidase comprises the amino acid sequence set forth in SEQ ID NO:885. In another embodiment, the recombinant human hyaluronidase consists of the amino acid sequence set forth in SEQ ID NO:885. In another embodiment, the recombinant human hyaluronidase comprises the amino acid sequence set forth in SEQ ID NO:886. In another embodiment, the recombinant human hyaluronidase consists of the amino acid sequence set forth in SEQ ID NO:886. In another embodiment, the recombinant human hyaluronidase comprises the amino acid sequence set forth in SEQ ID NO:887. In another embodiment, the recombinant human hyaluronidase consists of the amino acid sequence set forth in SEQ ID NO:887. In another embodiment, the recombinant human hyaluronidase comprises the amino acid sequence set forth in SEQ ID NO:888. In another embodiment, the recombinant human hyaluronidase consists of the amino acid sequence set forth in SEQ ID NO:888. In another embodiment, the recombinant human hyaluronidase comprises the amino acid sequence set forth in SEQ ID NO:889. In another embodiment, the recombinant human hyaluronidase consists of the amino acid sequence set forth in SEQ ID NO:889. In another embodiment, the recombinant human hyaluronidase comprises the amino acid sequence set forth in SEQ ID NO:890. In another embodiment, the recombinant human hyaluronidase consists of the amino acid sequence set forth in SEQ ID NO:890. In another embodiment, the recombinant human hyaluronidase comprises the amino acid sequence set forth in SEQ ID NO:891. In another embodiment, the recombinant human hyaluronidase consists of the amino acid sequence set forth in SEQ ID NO:891. In another embodiment, the recombinant human hyaluronidase comprises the amino acid sequence set forth in SEQ ID NO:892. In another embodiment, the recombinant human hyaluronidase consists of the amino acid sequence set forth in SEQ ID NO:892. In another embodiment, the recombinant human hyaluronidase comprises the amino acid sequence set forth in SEQ ID NO:893. In another embodiment, the recombinant human hyaluronidase consists of the amino acid sequence set forth in SEQ ID NO:893. In another embodiment, the recombinant human hyaluronidase comprises the amino acid sequence set forth in SEQ ID NO:894. In another embodiment, the recombinant human hyaluronidase consists of the amino acid sequence set forth in SEQ ID NO:894. In another embodiment, the recombinant human hyaluronidase comprises the amino acid sequence set forth in SEQ ID NO:895. In another embodiment, the recombinant human hyaluronidase consists of the amino acid sequence set forth in SEQ ID NO:895. In another embodiment, the recombinant human hyaluronidase comprises the amino acid sequence set forth in SEQ ID NO:896. In another embodiment, the recombinant human hyaluronidase consists of the amino acid sequence set forth in SEQ ID NO:896. In another embodiment, the recombinant human hyaluronidase comprises the amino acid sequence set forth in SEQ ID NO:897. In another embodiment, the recombinant human hyaluronidase consists of the amino acid sequence set forth in SEQ ID NO:897. In another embodiment, the recombinant human hyaluronidase comprises the amino acid sequence set forth in SEQ ID NO:898. In another embodiment, the recombinant human hyaluronidase consists of the amino acid sequence set forth in SEQ ID NO:898. In another embodiment, the recombinant human hyaluronidase comprises the amino acid sequence set forth in SEQ ID NO:899. In another embodiment, the recombinant human hyaluronidase consists of the amino acid sequence set forth in SEQ ID NO:899. In another embodiment, the recombinant human hyaluronidase comprises the amino acid sequence set forth in SEQ ID NO:900. In another embodiment, the recombinant human hyaluronidase consists of the amino acid sequence set forth in SEQ ID NO:900. In another embodiment, the recombinant human hyaluronidase comprises the amino acid sequence set forth in SEQ ID NO:901. In another embodiment, the recombinant human hyaluronidase consists of the amino acid sequence set forth in SEQ ID NO:901. In another embodiment, the recombinant human hyaluronidase comprises the amino acid sequence set forth in SEQ ID NO:902. In another embodiment, the recombinant human hyaluronidase consists of the amino acid sequence set forth in SEQ ID NO:902. In another embodiment, the recombinant human hyaluronidase comprises the amino acid sequence set forth in SEQ ID NO:903. In another embodiment, the recombinant human hyaluronidase consists of the amino acid sequence set forth in SEQ ID NO:903. In another embodiment, the recombinant human hyaluronidase comprises the amino acid sequence set forth in SEQ ID NO:904. In another embodiment, the recombinant human hyaluronidase consists of the amino acid sequence set forth in SEQ ID NO:904. In another embodiment, the recombinant human hyaluronidase comprises the amino acid sequence set forth in SEQ ID NO:905. In another embodiment, the recombinant human hyaluronidase consists of the amino acid sequence set forth in SEQ ID NO:905. In another embodiment, the recombinant human hyaluronidase comprises the amino acid sequence set forth in SEQ ID NO:906. In another embodiment, the recombinant human hyaluronidase consists of the amino acid sequence set forth in SEQ ID NO:906. In another embodiment, the recombinant human hyaluronidase comprises the amino acid sequence set forth in SEQ ID NO:907. In another embodiment, the recombinant human hyaluronidase consists of the amino acid sequence set forth in SEQ ID NO:907. In another embodiment, the recombinant human hyaluronidase comprises the amino acid sequence set forth in SEQ ID NO:908. In another embodiment, the recombinant human hyaluronidase consists of the amino acid sequence set forth in SEQ ID NO:908. In another embodiment, the recombinant human hyaluronidase comprises the amino acid sequence set forth in SEQ ID NO:909. In another embodiment, the recombinant human hyaluronidase consists of the amino acid sequence set forth in SEQ ID NO:909.

In certain embodiments, the hyaluronidase or variant thereof and the anti-OX40L antibody, or antigen binding fragment thereof, are administered in separate formulations. In certain embodiments, the hyaluronidase or variant thereof and the anti-OX40L antibody, or antigen binding fragment thereof, are administered simultaneously in separate formulations. In certain embodiments, the hyaluronidase or variant thereof is administered to the patient prior to administration of the anti-OX40L antibody, or antigen binding fragment thereof. In certain embodiments, the hyaluronidase or variant thereof is administered to the patient after administration of the anti-OX40L antibody, or antigen binding fragment thereof. In certain embodiments, the hyaluronidase or variant thereof and the anti-OX40L antibody, or antigen binding fragment thereof, are mixed and administered in a single formulation. In certain embodiments, the hyaluronidase or variant thereof and the anti-OX40L antibody, or antigen binding fragment thereof, are administered by subcutaneous injection. In certain embodiments, the hyaluronidase or variant thereof and the anti-OX40L antibody, or antigen binding fragment thereof, are administered by intravenous injection.

In certain embodiments, the hyaluronidase is rHuPH20 administered at a concentration of 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, or 40,000 units.

In certain embodiments, methods of treating an inflammatory disorder or disease in a human patient are provided, wherein the method comprises co-administering to the patient any one of the antibodies, or antigen binding fragments thereof, that bind OX40L described herein and a hyaluronidase or variant thereof. In certain embodiments, the inflammatory disorder or disease is atopic dermatitis. In certain embodiments, the treatment reduces disease severity in the patient and wherein disease severity is assessed by an atopic dermatitis disease severity outcome measure. In certain embodiments, the inflammatory disorder or disease is asthma, idiopathic pulmonary fibrosis, alopecia areata, chronic sinusitis with nasal polyps, Chronic Rhinosinusitis without Nasal Polyps (CRSsNP), eosinophilic esophagitis (EoE), Churg-Strauss syndrome/Eosinophilic granulomatosis with polyangiitis (EGPA), Prurigo Nodularis (PN), Chronic Spontaneous Urticaria (CSU), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), lupus, rheumatoid arthritis (RA), psoriasis, an inflammatory bowel disease, such as Crohn's disease or ulcerative colitis, hidradenitis suppurativa, celiac disease, systemic sclerosis, allergic rhinitis, eosinophilic fasciitis, scleromyxedema, scleredema, or nephrogenic systemic fibrosis. In certain embodiments, the inflammatory disorder or disease is an Eosinophilic gastrointestinal disorder or disease (EGID), such as Eosinophilic Gastritis (EoG), Eosinophilic Enteritis (EoN), Eosinophilic Colitis (EoC), or Eosinophilic Gastroenteritis (EGE).

In certain aspects, described herein are compositions comprising an antibody, or antigen binding fragment thereof, that binds OX40L wherein the antibody, or antigen binding fragment thereof, comprises a CDR-H1 comprising the sequence set forth in SEQ ID NO:1; a CDR-H2 comprising the sequence set forth in SEQ ID NO: 70; a CDR-H3 comprising the sequence set forth in SEQ ID NO: 175; a CDR-L1 comprising the sequence set forth in SEQ ID NO: 258; a CDR-L2 comprising the sequence set forth in SEQ ID NO: 335; and a CDR-L3 comprising the sequence set forth in SEQ ID NO: 368, and a hyaluronidase or variant thereof. In certain embodiments, the antibody, or antigen binding fragment thereof, that binds OX40L comprises a VH sequence set forth in SEQ ID NO: 419 and a VL sequence set forth in SEQ ID NO: 489. In certain embodiments, the antibody, or antigen binding fragment thereof, comprises a heavy chain comprising a constant heavy chain sequence set forth in any one of SEQ ID NOs: 637-737 and 913-1012. In certain embodiments, the antibody, or antigen binding fragment thereof, comprises a heavy chain comprising a constant heavy chain sequence set forth in SEQ ID NO: 651 or SEQ ID NO: 927. In certain embodiments, the antibody, or antigen binding fragment thereof, comprises a heavy chain comprising a constant heavy chain sequence set forth in SEQ ID NO: 651. In certain embodiments, the antibody, or antigen binding fragment thereof, comprises a heavy chain comprising a constant heavy chain sequence set forth in SEQ ID NO: 927. In certain embodiments, the antibody, or antigen binding fragment thereof, comprises a light chain comprising a constant light chain sequence set forth in SEQ ID NO: 738. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 927 and/or a constant heavy chain sequence set forth in SEQ ID NO: 651, and a constant light chain sequence set forth in SEQ ID NO: 738. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 927, and a constant light chain sequence set forth in SEQ ID NO: 738. In certain embodiments, the antibody, or antigen binding fragment thereof, comprises a heavy chain sequence set forth in SEQ ID NO: 1013 and a light chain sequence set forth in SEQ ID NO: 1015. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 651, and a constant light chain sequence set forth in SEQ ID NO: 738. In certain embodiments, the antibody, or antigen binding fragment thereof, comprises a heavy chain sequence set forth in SEQ ID NO: 1014 and a light chain sequence set forth in SEQ ID NO: 1015.

In certain aspects, described herein are compositions comprising an antibody, or antigen binding fragment thereof, that binds OX40L wherein the antibody, or antigen binding fragment thereof, comprises a CDR-H1 comprising the sequence set forth in SEQ ID NO: 44; a CDR-H2 comprising the sequence set forth in SEQ ID NO: 910; a CDR-H3 comprising the sequence set forth in SEQ ID NO: 911; a CDR-L1 comprising the sequence set forth in SEQ ID NO: 334; a CDR-L2 comprising the sequence set forth in SEQ ID NO: 361; and a CDR-L3 comprising the sequence set forth in SEQ ID NO: 912, and a hyaluronidase or variant thereof. In certain embodiments, the antibody, or antigen binding fragment thereof, that binds OX40L comprises a VH sequence set forth in SEQ ID NO: 832 and a VL sequence set forth in SEQ ID NO: 833. In certain embodiments, the antibody, or antigen binding fragment thereof comprises a heavy chain comprising a constant heavy chain sequence selected from a sequence set forth in any one of SEQ ID NOs: 637-737 and 913-1012. In certain embodiments, the antibody has a heavy chain sequence set forth in SEQ ID NO: 1018 and a light chain sequence set forth in SEQ ID NO: 1019. In certain embodiments, the antibody, or antigen binding fragment thereof comprises a heavy chain comprising a constant heavy chain sequence selected from a sequence set forth in any one of SEQ ID NOs: 637-639, 641-737, 913-915, and 917-1012. In certain embodiments, the antibody, or antigen binding fragment thereof does not comprise a constant heavy chain sequence set forth in SEQ ID NOs: 640 and 916 (i.e., a human IgG4-SPLE constant heavy chain region).

In certain aspects, described herein are a combination of an antibody, or antigen binding fragment thereof, that binds, wherein the antibody, or antigen binding fragment thereof, comprises a CDR-H1 comprising the sequence set forth in SEQ ID NO:1; a CDR-H2 comprising the sequence set forth in SEQ ID NO: 70; a CDR-H3 comprising the sequence set forth in SEQ ID NO: 175; a CDR-L1 comprising the sequence set forth in SEQ ID NO: 258; a CDR-L2 comprising the sequence set forth in SEQ ID NO: 335; and a CDR-L3 comprising the sequence set forth in SEQ ID NO: 368, and a hyaluronidase or variant thereof. In certain embodiments, the antibody, or antigen binding fragment thereof, that binds OX40L comprises a VH sequence set forth in SEQ ID NO: 419 and a VL sequence set forth in SEQ ID NO: 489. In certain embodiments, the antibody, or antigen binding fragment thereof, comprises a heavy chain comprising a constant heavy chain sequence set forth in any one of SEQ ID NOs: 637-737 and 913-1012. In certain embodiments, the antibody, or antigen binding fragment thereof, comprises a heavy chain comprising a constant heavy chain sequence set forth in SEQ ID NO: 651 or SEQ ID NO: 927. In certain embodiments, the antibody, or antigen binding fragment thereof, comprises a heavy chain comprising a constant heavy chain sequence set forth in SEQ ID NO: 651. In certain embodiments, the antibody, or antigen binding fragment thereof, comprises a heavy chain comprising a constant heavy chain sequence set forth in SEQ ID NO: 927. In certain embodiments, the antibody, or antigen binding fragment thereof, comprises a light chain comprising a constant light chain sequence set forth in SEQ ID NO: 738. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 927 and/or a constant heavy chain sequence set forth in SEQ ID NO: 651, and a constant light chain sequence set forth in SEQ ID NO: 738. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 927, and a constant light chain sequence set forth in SEQ ID NO: 738. In certain embodiments, the antibody, or antigen binding fragment thereof, comprises a heavy chain sequence set forth in SEQ ID NO: 1013 and a light chain sequence set forth in SEQ ID NO: 1015. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 651, and a constant light chain sequence set forth in SEQ ID NO: 738. In certain embodiments, the antibody, or antigen binding fragment thereof, comprises a heavy chain sequence set forth in SEQ ID NO: 1014 and a light chain sequence set forth in SEQ ID NO: 1015.

In certain aspects, described herein are a combination of an antibody, or antigen binding fragment thereof, that binds, wherein the antibody comprises a CDR-H1 comprising the sequence set forth in SEQ ID NO: 44; a CDR-H2 comprising the sequence set forth in SEQ ID NO: 910; a CDR-H3 comprising the sequence set forth in SEQ ID NO: 911; a CDR-L1 comprising the sequence set forth in SEQ ID NO: 334; a CDR-L2 comprising the sequence set forth in SEQ ID NO: 361; and a CDR-L3 comprising the sequence set forth in SEQ ID NO: 912, and a hyaluronidase or variant thereof. In certain embodiments, the antibody, or antigen binding fragment thereof, that binds OX40L comprises a VH sequence set forth in SEQ ID NO: 832 and a VL sequence set forth in SEQ ID NO: 833. In certain embodiments, the antibody, or antigen binding fragment thereof comprises a heavy chain comprising a constant heavy chain sequence selected from a sequence set forth in any one of SEQ ID NOs: 637-737 and 913-1012. In certain embodiments, the antibody has a heavy chain sequence set forth in SEQ ID NO: 1018 and a light chain sequence set forth in SEQ ID NO: 1019. In certain embodiments, the antibody, or antigen binding fragment thereof comprises a heavy chain comprising a constant heavy chain sequence selected from a sequence set forth in any one of SEQ ID NOs: 637-639, 641-737, 913-915, and 917-1012. In certain embodiments, the antibody, or antigen binding fragment thereof does not comprise a constant heavy chain sequence set forth in SEQ ID NOs: 640 and 916 (i.e., a human IgG4-SPLE constant heavy chain region).

In certain aspects, described herein are methods of treating an inflammatory disorder or disease in a human patient comprising co-administering to the patient an antibody, or antigen binding fragment thereof, that binds OX40L, wherein the antibody, or antigen binding fragment thereof, comprises a CDR-H1 comprising the sequence set forth in SEQ ID NO:1; a CDR-H2 comprising the sequence set forth in SEQ ID NO: 70; a CDR-H3 comprising the sequence set forth in SEQ ID NO: 175; a CDR-L1 comprising the sequence set forth in SEQ ID NO: 258; a CDR-L2 comprising the sequence set forth in SEQ ID NO: 335; and a CDR-L3 comprising the sequence set forth in SEQ ID NO: 368, and a hyaluronidase or variant thereof. In certain embodiments, the antibody, or antigen binding fragment thereof, that binds OX40L comprises a VH sequence set forth in SEQ ID NO: 419 and a VL sequence set forth in SEQ ID NO: 489. In certain embodiments, the antibody, or antigen binding fragment thereof, comprises a heavy chain comprising a constant heavy chain sequence set forth in any one of SEQ ID NOs: 637-737 and 913-1012. In certain embodiments, the antibody, or antigen binding fragment thereof, comprises a heavy chain comprising a constant heavy chain sequence set forth in SEQ ID NO: 651 or SEQ ID NO: 927. In certain embodiments, the antibody, or antigen binding fragment thereof, comprises a heavy chain comprising a constant heavy chain sequence set forth in SEQ ID NO: 651. In certain embodiments, the antibody, or antigen binding fragment thereof, comprises a heavy chain comprising a constant heavy chain sequence set forth in SEQ ID NO: 927. In certain embodiments, the antibody, or antigen binding fragment thereof, comprises a light chain comprising a constant light chain sequence set forth in SEQ ID NO: 738. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 927 and/or a constant heavy chain sequence set forth in SEQ ID NO: 651, and a constant light chain sequence set forth in SEQ ID NO: 738. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 927, and a constant light chain sequence set forth in SEQ ID NO: 738. In certain embodiments, the antibody, or antigen binding fragment thereof, comprises a heavy chain sequence set forth in SEQ ID NO: 1013 and a light chain sequence set forth in SEQ ID NO: 1015. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 651, and a constant light chain sequence set forth in SEQ ID NO: 738. In certain embodiments, the antibody, or antigen binding fragment thereof, comprises a heavy chain sequence set forth in SEQ ID NO: 1014 and a light chain sequence set forth in SEQ ID NO: 1015.

In other aspects, described herein are methods of treating an inflammatory disorder or disease in a human patient comprising co-administering to the patient an antibody, or antigen binding fragment thereof, that binds OX40L, wherein the antibody, or antigen binding fragment thereof, comprises a CDR-H1 comprising the sequence set forth in SEQ ID NO: 44; a CDR-H2 comprising the sequence set forth in SEQ ID NO: 910; a CDR-H3 comprising the sequence set forth in SEQ ID NO: 911; a CDR-L1 comprising the sequence set forth in SEQ ID NO: 334; a CDR-L2 comprising the sequence set forth in SEQ ID NO: 361; and a CDR-L3 comprising the sequence set forth in SEQ ID NO: 912, and a hyaluronidase or variant thereof. In certain embodiments, the antibody, or antigen binding fragment thereof, that binds OX40L comprises a VH sequence set forth in SEQ ID NO: 832 and a VL sequence set forth in SEQ ID NO: 833. In certain embodiments, the antibody, or antigen binding fragment thereof comprises a heavy chain comprising a constant heavy chain sequence selected from a sequence set forth in any one of SEQ ID NOs: 637-737 and 913-1012. In certain embodiments, the antibody has a heavy chain sequence set forth in SEQ ID NO: 1018 and a light chain sequence set forth in SEQ ID NO: 1019. In certain embodiments, the antibody, or antigen binding fragment thereof comprises a heavy chain comprising a constant heavy chain sequence selected from a sequence set forth in any one of SEQ ID NOs: 637-639, 641-737, 913-915, and 917-1012. In certain embodiments, the antibody, or antigen binding fragment thereof does not comprise a constant heavy chain sequence set forth in SEQ ID NOs: 640 and 916 (i.e., a human IgG4-SPLE constant heavy chain region).

In certain aspects, described herein are kits for treating an inflammatory disorder or disease in a human patient, the kit comprising: (a) a dose of any one of the antibodies, or antigen binding fragments thereof, that binds OX40L described herein; (b) a dose of a hyaluronidase or variant thereof, and (c) instructions. In certain embodiments, the antibody, or antigen binding fragment thereof, that binds OX40L comprises a CDR-H1 comprising the sequence set forth in SEQ ID NO:1; a CDR-H2 comprising the sequence set forth in SEQ ID NO: 70; a CDR-H3 comprising the sequence set forth in SEQ ID NO: 175; a CDR-L1 comprising the sequence set forth in SEQ ID NO: 258; a CDR-L2 comprising the sequence set forth in SEQ ID NO: 335; and a CDR-L3 comprising the sequence set forth in SEQ ID NO: 368. In certain embodiments, the antibody, or antigen binding fragment thereof, that binds OX40L comprises a CDR-H1 comprising the sequence set forth in SEQ ID NO: 44; a CDR-H2 comprising the sequence set forth in SEQ ID NO: 910; a CDR-H3 comprising the sequence set forth in SEQ ID NO: 911; a CDR-L1 comprising the sequence set forth in SEQ ID NO: 334; a CDR-L2 comprising the sequence set forth in SEQ ID NO: 361; and a CDR-L3 comprising the sequence set forth in SEQ ID NO: 912. In certain embodiments, the antibody, or antigen binding fragment thereof, that binds OX40L comprises a VH sequence set forth in SEQ ID NO: 419 and a VL sequence set forth in SEQ ID NO: 489. In certain embodiments, the antibody, or antigen binding fragment thereof, that binds OX40L comprises a VH sequence set forth in SEQ ID NO: 832 and a VL sequence set forth in SEQ ID NO: 833. In certain embodiments, the antibody, or antigen binding fragment thereof, comprises a heavy chain comprising a constant heavy chain sequence set forth in any one of SEQ ID NOs: 637-737 and 913-1012. In certain embodiments, the antibody, or antigen binding fragment thereof, comprises a heavy chain comprising a constant heavy chain sequence set forth in SEQ ID NO: 651 or SEQ ID NO: 927. In certain embodiments, the antibody, or antigen binding fragment thereof, comprises a heavy chain comprising a constant heavy chain sequence set forth in SEQ ID NO: 651. In certain embodiments, the antibody, or antigen binding fragment thereof, comprises a heavy chain comprising a constant heavy chain sequence set forth in SEQ ID NO: 927. In certain embodiments, the antibody, or antigen binding fragment thereof, comprises a light chain comprising a constant light chain sequence set forth in SEQ ID NO: 738. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 927 and/or a constant heavy chain sequence set forth in SEQ ID NO: 651, and a constant light chain sequence set forth in SEQ ID NO: 738. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 927, and a constant light chain sequence set forth in SEQ ID NO: 738. In certain embodiments, the antibody, or antigen binding fragment thereof, comprises a heavy chain sequence set forth in SEQ ID NO: 1013 and a light chain sequence set forth in SEQ ID NO: 1015. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 651, and a constant light chain sequence set forth in SEQ ID NO: 738. In certain embodiments, the antibody, or antigen binding fragment thereof, comprises a heavy chain sequence set forth in SEQ ID NO: 1014 and a light chain sequence set forth in SEQ ID NO: 1015.

In certain embodiments, the hyaluronidase is a soluble hyaluronidase. In certain embodiments, the hyaluronidase is a recombinant human hyaluronidase or variant thereof. In certain embodiments, the hyaluronidase is a recombinant human hyaluronidase. In certain embodiments, the recombinant human hyaluronidase comprises the amino acid sequence set forth in any one of SEQ ID NOs: 843-852 and 854-909, or a mixture thereof. In certain embodiments, the recombinant human hyaluronidase is rHuPH20 (i.e., a composition comprising one or more of SEQ ID NOs: 845-850). In certain embodiments, the rHuPH20 formulation is ENHANZE®.

In certain embodiments, the kit is for use in treating an inflammatory disorder or disease. In certain embodiments, the inflammatory disorder or disease is atopic dermatitis. In certain embodiments, the inflammatory disorder or disease is asthma, idiopathic pulmonary fibrosis, alopecia areata, chronic sinusitis with nasal polyps, Chronic Rhinosinusitis without Nasal Polyps (CRSsNP), eosinophilic esophagitis (EoE), Churg-Strauss syndrome/Eosinophilic granulomatosis with polyangiitis (EGPA), Prurigo Nodularis (PN), Chronic Spontaneous Urticaria (CSU), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), lupus, rheumatoid arthritis (RA), psoriasis, an inflammatory bowel disease, such as Crohn's disease or ulcerative colitis, hidradenitis suppurativa, celiac disease, systemic sclerosis, allergic rhinitis, eosinophilic fasciitis, scleromyxedema, scleredema, or nephrogenic systemic fibrosis. In certain embodiments, the inflammatory disorder or disease is an Eosinophilic gastrointestinal disorder or disease (EGID), such as Eosinophilic Gastritis (EoG), Eosinophilic Enteritis (EoN), Eosinophilic Colitis (EoC), or Eosinophilic Gastroenteritis (EGE).

### DETAILED DESCRIPTION

Described herein are compositions and combinations containing anti-OX40L antibodies, or antigen binding fragments thereof, and methods of use thereof. The compositions and combinations containing an anti-OX40L antibody, or antigen binding fragment thereof, described herein further comprise a hyaluronidase or a variant thereof. Also described herein are methods of treating a subject having an inflammatory disorder or disease, such as atopic dermatitis, by administering an anti-OX40L antibody, or antigen binding fragment thereof, described herein and a hyaluronidase or a variant thereof. The anti-OX40L antibody, or antigen binding fragment thereof, and the hyaluronidase or a variant thereof may be administered separately or may be co-formulated and administered in a single composition.

### Definitions

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4^{th} ed. (2012) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

As used herein, the singular forms “a,” “an,” and “the” include plural references unless indicated otherwise.

It is understood that aspects and embodiments of the invention described herein include “comprising,” “consisting,” and “consisting essentially of” aspects and embodiments.

For all compositions described herein, and all methods using a composition described herein, the compositions can either comprise the listed components or steps, or can “consist essentially of” the listed components or steps. When a composition is described as “consisting essentially of” the listed components, the composition contains the components listed and may contain other components which do not substantially affect the condition being treated, but do not contain any other components which substantially affect the condition being treated other than those components expressly listed; or, if the composition does contain extra components other than those listed which substantially affect the condition being treated, the composition does not contain a sufficient concentration or amount of the extra components to substantially affect the condition being treated. When a method is described as “consisting essentially of” the listed steps, the method contains the steps listed, and may contain other steps that do not substantially affect the condition being treated, but the method does not contain any other steps which substantially affect the condition being treated other than those steps expressly listed. As a non-limiting specific example, when a composition is described as “consisting essentially of” a component, the composition may additionally contain any amount of pharmaceutically acceptable carriers, vehicles, or diluents and other such components which do not substantially affect the condition being treated.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably herein and refer to cells into which an exogenous nucleic acid has been introduced, and the progeny of such cells. Host cells include “transformants” (or “transformed cells”) and “transfectants” (or “transfected cells”), which each include the primary transformed or transfected cell and progeny derived therefrom. Such progeny may not be completely identical in nucleic acid content to a parent cell, and may contain mutations. A “recombinant host cell” or “host cell” refers to a cell that includes an exogenous polynucleotide, regardless of the method used for insertion, for example, direct uptake, transduction, f-mating, or other methods known in the art to create recombinant host cells.

As used herein, the term “eukaryote” refers to organisms belonging to the phylogenetic domain Eukarya such as animals (including but not limited to, mammals, insects, reptiles, birds, etc.), ciliates, plants (including but not limited to, monocots, dicots, algae, etc.), fungi, yeasts, flagellates, microsporidia, protists, etc.

As used herein, the term “prokaryote” refers to prokaryotic organisms. For example, a non-eukaryotic organism can belong to the Eubacteria (including but not limited to, Escherichia coli, Thermus thermophilus, Bacillus stearothermophilus, Pseudomonas fluorescens, Pseudomonas aeruginosa, Pseudomonas putida, etc.) phylogenetic domain, or the Archaea (including but not limited to, Methanococcus jannaschii, Methanobacterium thermoautotrophicum, Halobacterium such as Haloferax volcanii and Halobacterium species NRC-1, Archaeoglobus fulgidus, Pyrococcus furiosus, Pyrococcus horikoshii, Aeuropyrum pernix, etc.) phylogenetic domain.

An “effective amount” or “therapeutically effective amount” as used herein refers to an amount of therapeutic compound, such as an anti-OX40L antibody, or antigen binding fragment thereof, administered to an individual, either as a single dose or as part of a series of doses, which is effective to produce or contribute to a desired therapeutic effect, either alone or in combination with another therapeutic modality. Examples of a desired therapeutic effect are reducing an aberrant immune response, slowing or delaying disease development; stabilization of disease; and amelioration of one or more symptoms. An effective amount may be given in one or more dosages.

The term “treating” (and variations thereof such as “treat” or “treatment”) refers to clinical intervention in an attempt to alter the natural course of a disease or condition in a subject in need thereof. Treatment can be performed during the course of clinical pathology. Desirable effects of treatment include preventing recurrence of disease, alleviation of symptoms, diminishment of any direct or indirect pathological consequences of the disease, preventing metastasis, decreasing the rate of disease progression, amelioration or palliation of the disease state, and remission or improved prognosis.

The term “sufficient amount” means an amount sufficient to produce a desired effect, e.g., an amount sufficient to modulate an immune response in a subject.

As used herein, the terms “subject,” “patient,” and “individual” are used interchangeably herein and each term means a mammalian subject. Exemplary subjects include humans, monkeys, dogs, cats, mice, rats, cows, horses, camels, goats, rabbits, and sheep. In certain embodiments, the subject is a human. In some embodiments the subject has a disease or condition that can be treated with an antibody provided herein. In some aspects, the disease or condition is atopic dermatitis. In some aspects, the disease or condition is asthma.

The term “in vitro” refers to processes that occur in a living cell growing separate from a living organism, e.g., growing in tissue culture.

The term “in vivo” refers to processes that occur in a living organism.

The term “package insert” is used to refer to instructions customarily included in commercial packages of therapeutic or diagnostic products (e.g., kits) that contain information about the indications, usage, dosage, administration, combination therapy, contraindications and/or warnings concerning the use of such therapeutic or diagnostic products.

The term “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

As used herein, the term “pharmaceutically acceptable” refers to those compounds, materials, compositions, and/or dosage forms, which are suitable for contact with the tissues of a subject, such as a mammal (e.g., a human) without excessive toxicity, irritation, allergic response, and other problem complications commensurate with a reasonable benefit/risk ratio. Preferably, the term “pharmaceutically acceptable” means approved by a regulatory agency of the Federal or a state government or listed in the U.S. Pharmacopeia or other generally recognized pharmacopeia for use in mammals and more particularly in humans.

The terms “co-administration,” “co-administer,” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to the administration of a second therapeutic agent.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The term “about” indicates and encompasses an indicated value and a range above and below that value. In certain embodiments, the term “about” indicates the designated value ±10%, +5%, or +1%. In certain embodiments, where applicable, the term “about” indicates the designated value(s) ±one standard deviation of that value(s).

The term “agonize” refers to the activation of receptor signaling to induce a biological response associated with activation of the receptor. An “agonist” is an entity that binds to and agonizes a receptor.

The term “antagonize” refers to the inhibition of receptor signaling to inhibit a biological response associated with activation of the receptor. An “antagonist” is an entity that binds to and antagonizes a receptor.

For any of the structural and functional characteristics described herein, methods of determining these characteristics are known in the art.

The term “optionally” is meant, when used sequentially, to include from one to all of the enumerated combinations and contemplates all sub-combinations.

The term “amino acid” refers to, for example, the twenty common naturally occurring amino acids. Naturally occurring amino acids include alanine (Ala; A), arginine (Arg; R), asparagine (Asn; N), aspartic acid (Asp; D), cysteine (Cys; C), glutamic acid (Glu; E), glutamine (Gln; Q), Glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The term “affinity” refers to the strength of the sum total of non-covalent interactions between a single binding site of a molecule (e.g., an antibody) and its binding partner (e.g., an antigen or epitope). Unless indicated otherwise, as used herein, “affinity” refers to intrinsic binding affinity, which reflects a 1:1 interaction between members of a binding pair (e.g., antibody and antigen or epitope).

The term “kd” (sec-1), as used herein, refers to the dissociation rate constant of a particular antibody-antigen interaction. This value is also referred to as the koff value.

The term “ka” (M-1×sec-1), as used herein, refers to the association rate constant of a particular antibody-antigen interaction. This value is also referred to as the kon value.

The term “KD” (M), as used herein, refers to the dissociation equilibrium constant of a particular antibody-antigen interaction. KD=kd/ka. In some embodiments, the affinity of an antibody is described in terms of the KD for an interaction between such antibody and its antigen. For clarity, as known in the art, a smaller KD value indicates a higher affinity interaction, while a larger KD value indicates a lower affinity interaction.

The term “KA” (M-1), as used herein, refers to the association equilibrium constant of a particular antibody-antigen interaction. KA=ka/kd.

As used herein, “administration” refers to providing or giving a subject a therapeutic agent (e.g., an anti-OX40L antibody, or an antigen binding fragment thereof, described herein) by any effective route. Exemplary routes of administration are described herein below.

As used herein, the term “polypeptide” describes a single polymer in which the monomers are amino acid residues which are covalently conjugated together through amide bonds. A polypeptide is intended to encompass any amino acid sequence, either naturally occurring, recombinant, or synthetically produced.

As used herein, the terms “nucleic acid” and “polynucleotide,” used interchangeably herein, refer to a polymeric form of nucleosides in any length. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. The term encompasses molecules comprising nucleosides or nucleoside analogs containing chemically- or biologically-modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided.

As used herein, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally occurring amino acids in TABLE 1 below.

TABLE 1
Representative physicochemical properties
of naturally occurring amino acids

| | | | | Electrostatic | |
|---|---|---|---|---|---|
| | 3 | 1 | Side- | character at | |
| | Letter | Letter | chain | physiological | Steric |
| Amino Acid | Code | Code | Polarity | pH (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic | Glu | E | polar | anionic | intermediate |
| acid | | | | | |
| Glutamine | Gln | Q | polar | neutral | intermediate |
| Glycine | Gly | G | nonpolar | neutral | small |
| Histidine | His | H | polar | Both neutral | large |
| | | | | and cationic | |
| | | | | forms in | |
| | | | | equilibrium | |
| | | | | at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | nonpolar | neutral | intermediate |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in cubic angstroms: 50-100 is small, 100-150 is intermediate, 150-200 is large, and >200 is bulky

From TABLE 1 it is appreciated that the conservative amino acid families include (i) G, A, V, L, and I; (ii) D and E; (iii) C, S, and T; (iv) H, K, and R; (v) N and Q; and (vi) F, Y, and W. A conservative mutation or substitution is therefore one that substitutes one amino acid for a member of the same amino acid family (e.g., a substitution of Ser for Thr or Lys for Arg).

The term “antibody” is used herein in its broadest sense and includes certain types of immunoglobulin molecules comprising one or more antigen-binding domains that specifically bind to an antigen or epitope. An antibody specifically includes intact antibodies (e.g., intact immunoglobulins), antibody fragments, and multi-specific antibodies.

A “anti-OX40L antibody,” “OX40L antibody,” or “OX40L specific antibody” is an antibody, as provided herein, which specifically binds to the antigen OX40L.

The term “epitope” means a portion of an antigen that specifically binds to an antibody.

The term “hypervariable region” or “HVR,” as used herein, refers to each of the regions of an antibody variable domain which are hypervariable in sequence and/or form structurally defined loops (“hypervariable loops”).

The term “antigen-binding domain” means the portion of an antibody that is capable of specifically binding to an antigen or epitope.

The term “chimeric antibody” refers to an antibody in which a portion of the heavy and/or light chain is derived from a particular source or species, while the remainder of the heavy and/or light chain is derived from a different source or species.

The term “human antibody” refers to an antibody which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

The term “humanized antibody” refers to a protein having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject.

The term “multispecific antibody” refers to an antibody that comprises two or more different antigen-binding domains that collectively specifically bind two or more different epitopes.

A “monospecific antibody” is an antibody that comprises one or more binding sites that specifically bind to a single epitope. An example of a monospecific antibody is a naturally occurring IgG molecule which, while divalent (i.e., having two antigen-binding domains), recognizes the same epitope at each of the two antigen-binding domains. The binding specificity may be present in any suitable valency.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “single-chain” refers to a molecule comprising amino acid monomers linearly linked by peptide bonds. In a particular such embodiment, the C-terminus of the Fab light chain is connected to the N-terminus of the Fab heavy chain in the single-chain Fab molecule. As described in more detail herein, an scFv has a variable domain of light chain (VL) connected from its C-terminus to the N-terminal end of a variable domain of heavy chain (VH) by a polypeptide chain. Alternately the scFv comprises of polypeptide chain where in the C-terminal end of the VH is connected to the N-terminal end of VL by a polypeptide chain.

The “Fab fragment” (also referred to as fragment antigen-binding) contains the constant domain (CL) of the light chain and the first constant domain (CH1) of the heavy chain along with the variable domains VL and VH on the light and heavy chains respectively. The variable domains comprise the complementarity determining loops (CDR, also referred to as hypervariable region) that are involved in antigen-binding. Fab′ fragments differ from Fab fragments by the addition of a few residues at the carboxy terminus of the heavy chain CH1 domain including one or more cysteines from the antibody hinge region.

“F(ab′)2” fragments contain two Fab′ fragments joined, near the hinge region, by disulfide bonds. F(ab′)2 fragments may be generated, for example, by recombinant methods or by pepsin digestion of an intact antibody. The F(ab′) fragments can be dissociated, for example, by treatment with 8-mercaptoethanol.

“Fv” fragments comprise a non-covalently-linked dimer of one heavy chain variable domain and one light chain variable domain.

“Single-chain Fv” or “sFv” or “scFv” includes the VH and VL domains of an antibody, wherein these domains are present in a single polypeptide chain. In one embodiment, the Fv polypeptide further comprises a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds., Springer-Verlag, New York, pp. 269-315 (1994).

“scFv-Fc” fragments comprise an scFv attached to an Fc domain. For example, an Fc domain may be attached to the C-terminal of the scFv. The Fc domain may follow the VH or VL, depending on the orientation of the variable domains in the scFv (i.e., VH-VL or VL-VH). Any suitable Fc domain known in the art or described herein may be used. In some cases, the Fc domain comprises an IgG4 Fc domain.

The term “single domain antibody” or “sdAb” refers to a molecule in which one variable domain of an antibody specifically binds to an antigen without the presence of the other variable domain. Single domain antibodies, and fragments thereof, are described in Arabi Ghahroudi et al., FEBS Letters, 1998, 414:521-526 and Muyldermans et al., Trends in Biochem. Sci., 2001, 26:230-245, each of which is incorporated by reference in its entirety. Single domain antibodies are also known as sdAbs or nanobodies. SdAbs are fairly stable and easy to express as fusion partner with the Fc chain of an antibody (Harmsen M M, De Haard H J (2007). “Properties, production, and applications of camelid single-domain antibody fragments,” Appl. Microbiol Biotechnol. 77(1): 13-22).

The terms “full length antibody,” “intact antibody,” and “whole antibody” are used herein interchangeably to refer to an antibody having a structure substantially similar to a naturally occurring antibody structure and having heavy chains that comprise an Fc region. For example, when used to refer to an IgG molecule, a “full length antibody” is an antibody that comprises two heavy chains and two light chains.

The term “antibody fragment” refers to an antibody that comprises a portion of an intact antibody, such as the antigen-binding or variable region of an intact antibody. Antibody fragments include, for example, Fv fragments, Fab fragments, F(ab′)2 fragments, Fab′ fragments, scFv (sFv) fragments, and scFv-Fc fragments.

The term “Fc domain” or “Fc region” herein is used to define a C-terminal region of an immunoglobulin heavy chain that contains at least a portion of the constant region. The term includes native sequence Fc regions and variant Fc regions.

The term “substantially purified” refers to a construct described herein, or variant thereof that may be substantially or essentially free of components that normally accompany or interact with the protein as found in its naturally occurring environment, i.e. a native cell, or host cell in the case of a recombinantly produced antibody that in certain embodiments, is substantially free of cellular material includes preparations of protein having less than about 30%, less than about 25%, less than about 20%, less than about 15%, less than about 10%, less than about 5%, less than about 4%, less than about 3%, less than about 2%, or less than about 1% (by dry weight) of contaminating protein.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., using publicly available computer software such as BLAST, BLASTP, BLASTN, BLAST-2, ALIGN, MEGALIGN (DNASTAR), CLUSTALW, CLUSTAL OMEGA, or MUSCLE software or other algorithms available to persons of skill) or by visual inspection. Software for performing BLAST analyses is publicly available through the National Center for Biotechnology Information (ncbi.nlm.nih.gov). Those skilled in the art can determine appropriate parameters for aligning sequences, including any algorithms needed to achieve maximal alignment over the full length of the sequences being compared. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

It must be noted that, as used in the specification and the appended claims, the singular forms “a,” “an” and “the” include plural referents unless the context clearly dictates otherwise.

The term “atopic dermatitis disease severity outcome measure” refers to a determination of certain signs, symptoms, features or parameters that have been associated with atopic dermatitis and that can be quantitatively or qualitatively assessed. Exemplary atopic dermatitis disease severity outcome measures include “Eczema Area and Severity Index” (EASI), “Severity Scoring of Atopic Dermatitis” (SCORAD), “Validated Investigator Global Assessment-Atopic Dermatitis” (vIGA-AD), “Investigator Global Assessment of Signs” (IGSA), Rajka/Langeland Atopic Dermatitis Severity Score, “Body Surface Area” (BSA), and patient-reported outcomes including Pruritus Visual Analog Scale (an aspect of disease severity assessed as part of SCORAD), Sleep Loss Visual Analog Scale (an aspect of disease severity assessed as part of SCORAD), Atopic Dermatitis Symptom Diary (ADSD), Atopic Dermatitis Impact Questionnaire (ADIQ), Dermatology Life Quality Index (DLQI) (Finlay and Khan, Clin Exper Dermatol 1994; 19:210), 5-D Itch Scale (Elman et al., Br J Dermatol 2010; 162(3):587-593), Itch Numeric Rating Scale (I-NRS) (see Naegeli et al., International Journal of Dermatology. 2015; 54(6):715-722; and Newton L, et al., J. Patient Rep. Outcomes. 2019; 3(1):42), and the Patient-Oriented Eczema Measure (POEM) (www.nottingham.ac.uk/research/groups/cebd/resources/poem.aspx).

### Hyaluronidases

As used herein, “hyaluronidase” refers to an enzyme that degrades hyaluronic acid, which constitutes an essential part of the extracellular matrix. Included in this definition are naturally occurring hyaluronidases, recombinant hyaluronidases, both human and from other sources, as well as variants thereof. Such hyaluronidases include, but are not limited to, nonhuman hyaluronidases, including bacterial hyaluronidases, bovine hyaluronidases, ovine hyaluronidases, and variants thereof. Reference to hyaluronidase refers to all forms, including variants.

Hyaluronidases were initially discovered in bacteria, and are now known to be widely distributed in nature and have been found in many different species, including insects, snakes, and mammals. Human hyaluronidase is present both in organs (e.g., testis, spleen, skin, eyes, liver, kidneys, uterus, and placenta) and in body fluids (e.g., tears, blood, and semen). In the human, six different hyaluronidases, HYAL1-4, HYAL-P1 and PH-20, have been identified. PH-20 exerts the strongest biologic activity, is found in high concentrations in the testicles, and can be localized on the head and the acrosome of human spermatozoa (see, e.g., Weber G C, et al., Adv. Exp. Med. Biol. 2019; 1148:255-277).

Hyaluronidases are classified into three categories according to their mechanism of action (see, e.g., Jung H., Arch. Plast. Surg. 2020 July; 47(4):297-300). First, Mammalian hyaluronidases are endo-β-N-acetylhexosaminidases that break down β-1,4 glycosidic linkages to form tetrasaccharides. Second, leech/hookworm hyaluronidases are endo-β-D-glucuronidases that break down β-1,3 glycosidic bonds to form pentasaccharides and hexasaccharides. Finally, microbial hyaluronidases are classified as hyaluronate lyases. Unlike other hyaluronidases, they do not catalyze hydrolysis reactions. Instead, they produce unsaturated disaccharides through a β-elimination reaction at β-1,4 glycosidic linkage.

Hyaluronidases can also be classified into two types according to the pH at which they are most active (see, e.g., Jung H., Arch. Plast. Surg. 2020 July; 47(4):297-300). Acid-active hyaluronidases are activated at a pH of 3 to 4. Neutral-active hyaluronidases, which include the hyaluronidase enzymes found in snake and bee venom, are activated at a pH of 5 to 8 (see, e.g., Cavallini M, et al., Aesthet. Surg. J. 2013; 33:1167-74).

Hyaluronidase use has become more diverse and widespread in clinical practice. Today, animal-derived bovine or ovine testicular hyaluronidases, as well as synthetic hyaluronidases, are clinically applied as adjuncts to increase the bioavailability of drugs, for the therapy of extravasations, or for the management of complications associated with the aesthetic injection of hyaluronic acid-based fillers.

Exemplary of hyaluronan degrading enzymes are hyaluronidases, and particular chondroitinases and lyases that have the ability to depolymerize hyaluronan. Exemplary chondroitinases that are hyaluronan degrading enzymes include, but are not limited to, chondroitin ABC lyase (also known as chondroitinase ABC), chondroitin AC lyase (also known as chondroitin sulfate lyase or chondroitin sulfate eliminase) and chondroitin C lyase. Chondroitin ABC lyase comprises two enzymes, chondroitin-sulfate-ABC endolyase (EC 4.2.2.20) and chondroitin-sulfate-ABC exolyase (EC 4.2.2.21). Exemplary chondroitin-sulfate-ABC endolyases and chondroitin-sulfate-ABC exolyases include, but are not limited to, those from Proteus vulgaris and Flavobacterium heparinum (the Proteus vulgaris chondroitin-sulfate-ABC endolyase (e.g., see Sato et al. (1994) Appl. Microbiol. Biotechnol. 41(1):39-46)). Exemplary chondroitinase AC enzymes from the bacteria include, but are not limited to, those from Flavobacterium heparinum, Victivallis vadensis, and Arthrobacter aurescens (see, e.g., Tkalec et al. (2000) Applied and Environmental Microbiology 66(1):29-35; Ernst et al. (1995) Critical Reviews in Biochemistry and Molecular Biology 30(5):387-444). Exemplary chondroitinase C enzymes from the bacteria include, but are not limited to, those from Streptococcus and Flavobacterium (Hibi et al. (1989) FEMS-Microbiol-Lett. 48(2):121-4; Michelacci et al. (1976) J. Biol. Chem. 251:1154-8; Tsuda et al. (1999) Eur. J. Biochem. 262:127-133).

Hyaluronidases include bacterial hyaluronidases (EC 4.2.2.1 or EC 4.2.99.1), hyaluronidases from leeches, other parasites, and crustaceans (EC 3.2.1.36), and mammalian-type hyaluronidases (EC 3.2.1.35). Hyaluronidases include any of non-human origin including, but not limited to, murine, canine, feline, leporine, avian, bovine, ovine, porcine, equine, piscine, ranine, bacterial, and any from leeches, other parasites, and crustaceans. Exemplary non-human hyaluronidases include hyaluronidases from cows (SEQ ID NOs:10, 11, 64 of U.S. Pat. No. 8,568,713) and BH55 (U.S. Pat. Nos. 5,747,027 and 5,827,721), yellow jacket wasp (SEQ ID NOs:12 and 13 of U.S. Pat. No. 8,568,713), honey bee (SEQ ID NO:14 of U.S. Pat. No. 8,568,713), white-face hornet (SEQ ID NO:15 of U.S. Pat. No. 8,568,713), paper wasp (SEQ ID NO:16 of U.S. Pat. No. 8,568,713), mouse (SEQ ID NOs:17-19, and 32 of U.S. Pat. No. 8,568,713), pig (SEQ ID NOs:20-21 of U.S. Pat. No. 8,568,713), rat (SEQ ID NOs:22-24, and 31 of U.S. Pat. No. 8,568,713), rabbit (SEQ ID NO:25 of U.S. Pat. No. 8,568,713), sheep (SEQ ID NOs:26, 27, 63 and 65 of U.S. Pat. No. 8,568,713), orangutan (SEQ ID NO:28 of U.S. Pat. No. 8,568,713), cynomolgus monkey (SEQ ID NO:29 of U.S. Pat. No. 8,568,713), guinea pig (SEQ ID NO:30 of U.S. Pat. No. 8,568,713), Arthrobacter sp. (strain FB24) (SEQ ID NO:67 of U.S. Pat. No. 8,568,713), Bdellovibrio bacteriovorus (SEQ ID NO:68 of U.S. Pat. No. 8,568,713), Propionibacterium acnes (SEQ ID NO:69 of U.S. Pat. No. 8,568,713), Streptococcus agalactiae ((SEQ ID NO:70 of U.S. Pat. No. 8,568,713); 18RS21 (SEQ ID NO:71 of U.S. Pat. No. 8,568,713); serotype Ia (SEQ ID NO:72 of U.S. Pat. No. 8,568,713); serotype III (SEQ ID NO:73 of U.S. Pat. No. 8,568,713), Staphylococcus aureus (strain COL) (SEQ ID NO:74 of U.S. Pat. No. 8,568,713); strain MRSA252 (SEQ ID NOs:75 and 76 of U.S. Pat. No. 8,568,713 of U.S. Pat. No. 8,568,713); strain MSSA476 (SEQ ID NO:77 of U.S. Pat. No. 8,568,713); strain NCTC 8325 (SEQ ID NO:78 of U.S. Pat. No. 8,568,713); strain bovine RF122 (SEQ ID NOs:79 and 80 of U.S. Pat. No. 8,568,713); strain USA300 (SEQ ID NO:81 of U.S. Pat. No. 8,568,713), Streptococcus pneumoniae (SEQ ID NO:82 of U.S. Pat. No. 8,568,713); strain ATCC BAA-255/R6 (SEQ ID NO:83 of U.S. Pat. No. 8,568,713); serotype 2, strain D39/NCTC 7466 (SEQ ID NO:84 of U.S. Pat. No. 8,568,713), Streptococcus pyogenes (serotype (SEQ ID NO:85 of U.S. Pat. No. 8,568,713); serotype M2, strain MGAS10270 (SEQ ID NO:86 of U.S. Pat. No. 8,568,713); serotype M4, strain MGAS10750 (SEQ ID NO:87 of U.S. Pat. No. 8,568,713); serotype M6 (SEQ ID NO:88 of U.S. Pat. No. 8,568,713); serotype M12, strain MGAS2096 (SEQ ID NOs:89 and 90 of U.S. Pat. No. 8,568,713); serotype M12, strain MGAS9429 (SEQ ID NO:91 of U.S. Pat. No. 8,568,713); serotype M28 (SEQ ID NO:92 of U.S. Pat. No. 8,568,713); Streptococcus suis (SEQ ID NOs:93-95 of U.S. Pat. No. 8,568,713); Vibrio fischeri (strain ATCC 700601/ES 114 (SEQ ID NO:96 of U.S. Pat. No. 8,568,713), and the Streptomyces hyaluronolyticus hyaluronidase enzyme, which is specific for hyaluronic acid and does not cleave chondroitin or chondroitin sulfate (Ohya, T. and Kaneko, Y. (1970) Biochim. Biophys. Acta 198:607). Hyaluronidases also include those of human origin. Exemplary human hyaluronidases include PH20 (SEQ ID NO:1 of U.S. Pat. No. 8,568,713), HYAL1 (SEQ ID NO:36 of U.S. Pat. No. 8,568,713), HYAL2 (SEQ ID NO:37 of U.S. Pat. No. 8,568,713), HYAL3 (SEQ ID NO:38 of U.S. Pat. No. 8,568,713), and HYAL4 (SEQ ID NO:36 of U.S. Pat. No. 8,568,713). The sequences and contents of U.S. Pat. No. 8,568,713 are expressly incorporated herein by reference. Also included amongst hyaluronidases are soluble hyaluronidases, including, ovine and bovine PH20, soluble human PH20 and soluble rHuPH20. Examples of commercially available bovine or ovine soluble hyaluronidases include Vitrase® (ovine hyaluronidase) and Amphadase® (bovine hyaluronidase).

Hyaluronidases as described herein include precursor hyaluronan degrading enzyme polypeptides and mature hyaluronan degrading enzyme polypeptides (such as those in which a signal sequence has been removed), truncated forms thereof that have activity, and includes allelic variants and species variants, variants encoded by splice variants, and other variants, including polypeptides that have at least 40%, 45%, 50%, 55%, 60%, 65%, 70%, 75%, 80%, 85%, 90%, 95%, 96%, 97%, 98%, 99% or more sequence identity to the polypeptides set forth in SEQ ID NOs:843-852 and 854-909. Hyaluronidases also include those that contain chemical or posttranslational modifications and those that do not contain chemical or posttranslational modifications. Such modifications include, but are not limited to, pegylation, albumination, glycosylation, farnesylation, carboxylation, hydroxylation, phosphorylation, and other polypeptide modifications known in the art.

As used herein, a soluble hyaluronidase refers to a polypeptide characterized by its solubility under physiologic conditions. Soluble hyaluronidases can be distinguished, for example, by its partitioning into the aqueous phase of a Triton X-114 solution warmed to 37° C. (Bordier et al., (1981) J. Biol. Chem., 256:1604-7). Membrane-anchored, such as lipid anchored hyaluronidases, will partition into the detergent rich phase, but will partition into the detergent-poor or aqueous phase following treatment with Phospholipase-C. Included among soluble hyaluronidases are membrane anchored hyaluronidases in which one or more regions associated with anchoring of the hyaluronidase to the membrane has been removed or modified, where the soluble form retains hyaluronidase activity. Soluble hyaluronidases include recombinant soluble hyaluronidases and those contained in or purified from natural sources.

As used herein, activity refers to a functional activity or activities of a polypeptide or portion thereof associated with a full-length (complete) protein. Functional activities include, but are not limited to, biological activity, catalytic or enzymatic activity, antigenicity (ability to bind or compete with a polypeptide for binding to an anti-polypeptide antibody), immunogenicity, ability to form multimers, and the ability to specifically bind to a receptor or ligand for the polypeptide.

As used herein, hyaluronidase activity refers to the ability to enzymatically catalyze the cleavage of hyaluronic acid. The United States Pharmacopeia (USP) XXII assay for hyaluronidase determines hyaluronidase activity indirectly by measuring the amount of higher molecular weight hyaluronic acid, or hyaluronan, (HA) substrate remaining after the enzyme is allowed to react with the HA for 30 min at 37° C. (USP XXII-NF XVII (1990) 644-645 United States Pharmacopeia Convention, Inc, Rockville, Md.). A Reference Standard solution can be used in an assay to ascertain the relative activity, in units, of any hyaluronidase. In vitro assays to determine the hyaluronidase activity of hyaluronidases, such as soluble rHuPH20, are known in the art. Exemplary assays include the microturbidity assay (see, e.g., Example 3 of U.S. Pat. No. 8,568,713) that measures cleavage of hyaluronic acid by hyaluronidase indirectly by detecting the insoluble precipitate formed when the uncleaved hyaluronic acid binds with serum albumin. Reference Standards can be used, for example, to generate a standard curve to determine the activity in Units of the hyaluronidase being tested.

As used herein, “functionally equivalent amount” or grammatical variations thereof, with reference to a hyaluronan degrading enzyme, refers to the amount of hyaluronan degrading enzyme that achieves the same effect as an amount (such as a known number of Units of hyaluronidase activity) of a reference enzyme, such as a hyaluronidase. For example, the activity of any hyaluronan degrading enzyme can be compared to the activity of rHuPH20 to determine the functionally equivalent amount of a hyaluronan degrading enzyme that would achieve the same effect as a known amount of rHuPH20. For example, the ability of a hyaluronan degrading enzyme to act as a spreading or diffusing agent can be assessed by injecting it into the lateral skin of mice with trypan blue (see, e.g., U.S. Pat. Publication No. 20050260186), and the amount of hyaluronan degrading enzyme required to achieve the same amount of diffusion as, for example, 100 units of a Hyaluronidase Reference Standard, can be determined. The amount of hyaluronan degrading enzyme required is, therefore, functionally equivalent to 100 units.

Exemplary hyaluronan degrading enzymes are hyaluronidases, particularly soluble hyaluronidases, such as a PH20, or a truncated or variant form thereof. The PH20 can be, for example, an ovine, bovine or truncated human PH20. The human PH20 mRNA transcript is normally translated to generate a 509 amino acid precursor polypeptide (SEQ ID NO:843; and replicated below) containing a 35 amino acid signal sequence at the N-terminus (amino acid residue positions 1-35) and a 19 amino acid glycosylphosphatidylinositol (GPI) anchor attachment signal sequence at the C-terminus (amino acid residue positions 491-509). The mature PH20 is, therefore, a 474 amino acid polypeptide set forth in SEQ ID NO:844. Following transport of the precursor polypeptide to the ER and removal of the signal peptide, the C-terminal GPI-attachment signal peptide is cleaved to facilitate covalent attachment of a GPI anchor to the newly-formed C-terminal amino acid at the amino acid position corresponding to position 490 of the precursor polypeptide set forth in SEQ ID NO:843. Thus, a 474 amino acid GPI-anchored mature polypeptide with an amino acid sequence set forth in SEQ ID NO:844 is produced.

The amino acid sequence of the human PH20 precursor polypeptide (SEQ ID NO: 843; 509 amino acids) is as follows:

MGVLKFKHIFFRSFVKSSGVSQIVFTFLLIPCCLTLNFRAPPVIPNVPF
LWAWNAPSEFCLGKFDEPLDMSLFSFIGSPRINATGQGVTIFYVDRLGY
YPYIDSITGVTVNGGIPQKISLQDHLDKAKKDITFYMPVDNLGMAVIDW
EEWRPTWARNWKPKDVYKNRSIELVQQQNVQLSLTEATEKAKQEFEKAG
KDFLVETIKLGKLLRPNHLWGYYLFPDCYNHHYKKPGYNGSCFNVEIKR
NDDLSWLWNESTALYPSIYLNTQQSPVAATLYVRNRVREAIRVSKIPDA
KSPLPVFAYTRIVFTDQVLKFLSQDELVYTFGETVALGASGIVIWGTLS
IMRSMKSCLLLDNYMETILNPYIINVTLAAKMCSQVLCQEQGVCIRKNW
NSSDYLHLNPDNFAIQLEKGGKFTVRGKPTLEDLEQFSEKFYCSCYSTL
SCKEKADVKDTDAVDVCIADGVCIDAFLKPPMETEEPQIFYNASPSTLS
ATMFIVSILFLIISSVASL.

The amino acid sequence of the mature PH20 polypeptide (SEQ ID NO: 844; 474 amino acids) is as follows:

LNFRAPPVIPNVPFLWAWNAPSEFCLGKFDEPLDMSLFSFIGSPRINAT
GQGVTIFYVDRLGYYPYIDSITGVTVNGGIPQKISLQDHLDKAKKDITF
YMPVDNLGMAVIDWEEWRPTWARNWKPKDVYKNRSIELVQQQNVQLSLT
EATEKAKQEFEKAGKDFLVETIKLGKLLRPNHLWGYYLFPDCYNHHYKK
PGYNGSCFNVEIKRNDDLSWLWNESTALYPSIYLNTQQSPVAATLYVRN
RVREAIRVSKIPDAKSPLPVFAYTRIVFTDQVLKFLSQDELVYTFGETV
ALGASGIVIWGTLSIMRSMKSCLLLDNYMETILNPYIINVTLAAKMCSQ
VLCQEQGVCIRKNWNSSDYLHLNPDNFAIQLEKGGKFTVRGKPTLEDLE
QFSEKFYCSCYSTLSCKEKADVKDTDAVDVCIADGVCIDAFLKPPMETE
EPQIFYNASPSTLSATMFIVSILFLIISSVASL.

Human PH20 exhibits hyaluronidase activity at both neutral and acid pH. In one aspect, human PH20 is the prototypical neutral-active hyaluronidase that is generally locked to the plasma membrane via a GPI anchor. In another aspect, PH20 is expressed on the inner acrosomal membrane where it has hyaluronidase activity at both neutral and acid pH. It appears that PH20 contains two catalytic sites at distinct regions of the polypeptide: the Peptide 1 and Peptide 3 regions (Cherr et al., (2001) Matrix Biology 20:515-525). Evidence suggests that the Peptide 1 region of PH20, which corresponds to amino acid positions 107-137 of the mature polypeptide set forth in SEQ ID NO:844 and positions 142-172 of the precursor polypeptide set forth in SEQ ID NO:843, is required for enzyme activity at neutral pH. Amino acids at positions 111 and 113 (corresponding to the mature PH20 polypeptide set forth in SEQ ID NO:844) within this region appear to be important for activity, as mutagenesis by amino acid replacement results in PH20 polypeptides with 3% hyaluronidase activity or undetectable hyaluronidase activity, respectively, compared to the wild-type PH20 (Arming et al., (1997) Eur. J. Biochem. 247:810-814).

The Peptide 3 region, which corresponds to amino acid positions 242-262 of the mature polypeptide set forth in SEQ ID NO:844, and positions 277-297 of the precursor polypeptide set forth in SEQ ID NO:843, appears to be important for enzyme activity at acidic pH. Within this region, amino acids at positions 249 and 252 of the mature PH20 polypeptide appear to be essential for activity, and mutagenesis of either one results in a polypeptide essentially devoid of activity (Arming et al., (1997) Eur. J. Biochem. 247:810-814).

In addition to the catalytic sites, PH20 also contains a hyaluronan-binding site. Experimental evidence suggest that this site is located in the Peptide 2 region, which corresponds to amino acid positions 205-235 of the precursor polypeptide set forth in SEQ ID NO: 843 and positions 170-200 of the mature polypeptide set forth in SEQ ID NO:844. This region is highly conserved among hyaluronidases and is similar to the heparin binding motif Mutation of the arginine residue at position 176 (corresponding to the mature PH20 polypeptide set forth in SEQ ID NO:844) to a glycine results in a polypeptide with only about 1% of the hyaluronidase activity of the wild type polypeptide (Arming et al., (1997) Eur. J. Biochem. 247:810-814).

There are seven potential N-linked glycosylation sites in human PH20 at N82, N166, N235, N254, N368, N393, N490 of the polypeptide exemplified in SEQ ID NO:843. Because amino acids 36 to 464 of SEQ ID NO:843 appear to contain the minimally active human PH20 hyaluronidase domain, the N-linked glycosylation site N-490 is not required for proper hyaluronidase activity. There are six disulfide bonds in human PH20. Two disulfide bonds between the cysteine residues C60 and C351 and between C224 and C238 of the polypeptide exemplified in SEQ ID NO:843 (corresponding to residues C25 and C316, and C189 and C203 of the mature polypeptide set forth in SEQ ID NO:844, respectively). A further four disulfide bonds are formed between the cysteine residues C376 and C387; between C381 and C435; between C437 and C443; and between C458 and C464 of the polypeptide exemplified in SEQ ID NO: 843 (corresponding to residues C341 and C352; between C346 and C400; between C402 and C408; and between C423 and C429 of the mature polypeptide set forth in SEQ ID NO:844, respectively).

As used herein, soluble recombinant human PH20 (rHuPH20) refers to a soluble form of human PH20 that is recombinantly expressed in Chinese Hamster Ovary (CHO) cells. Soluble human PH20 or sHuPH20 includes mature polypeptides lacking all or a portion of the glycosylphospatidylinositol (GPI) attachment site at the C-terminus such that upon expression, the polypeptides are soluble.

Soluble rHuPH20 is encoded by a nucleic acid that includes the signal sequence and is set forth in SEQ ID NO:853. Also included are DNA molecules that are allelic variants thereof and other soluble variants. The nucleic acid encoding soluble rHuPH20 is expressed in CHO cells which secrete the mature polypeptide.

SEQ ID NO: 853 encodes amino acids 1-482 of the human PH20 precursor polypeptide (amino acids 1-482 of SEQ ID NO: 843). Post translational processing removes the 35 amino acid signal sequence, leaving a 447 amino acid soluble recombinant human PH20 (SEQ ID NO: 845). As produced in the culture medium, there is heterogeneity at the C-terminus such that the product, designated rHuPH20, includes a mixture of species that can include any one or more of SEQ ID NOs: 845-850 in various abundance. Typically, rHuPH20 is produced in cells that facilitate correct N-glycosylation to retain activity, such as CHO cells (e.g., DG44 CHO cells). rHuPH20 may be produced by expressing a polynucleotide encoding amino acids 36-482 of SEQ ID NO: 843 in a cell, such as a CHO cell, and the polynucleotide encoding amino acids 36-482 of SEQ ID NO: 843 may be linked to the native signal sequence (amino acids 1-35 of SEQ ID NO: 843, as in SEQ ID NO: 853) or a heterologous signal sequence. The production of rHuPH20 is described in U.S. Pat. Nos. 8,568,713 and 8,343,487, each of which is incorporated herein by reference.

Exemplary sHuPH20 polypeptides include mature polypeptides having an amino acid sequence set forth in any one of SEQ ID NOs:845-852 and 854-909. Other variants can have 60%, 70%, 80%, 85%, 90%, 91%, 92%, 93%, 94%, 95%, 96%, 97%, 98%, 99% or more sequence identity to any one of SEQ ID NOs: 845-852 and 854-909, as long they retain a hyaluronidase activity and are soluble. Corresponding allelic variants and other variants also are included, including those corresponding to the precursor human PH20 polypeptide set forth in SEQ ID NO:843 and the mature human PH20 polypeptide set forth in SEQ ID NO: 844.

rHuPH20 was approved by the US Food and Drug Administration in 2005 as an adjuvant to increase the dispersion and absorption of other injected drugs. Recombinant human hyaluronidase PH20 is a transiently and locally-acting permeation enhancer that increases the dispersion and absorption of other injected agents. Recombinant human hyaluronidase PH20 depolymerizes hyaluronic acid (HA) at the injection site causing rapid decrease in the viscosity of the extracellular matrix, allowing bulk fluid flow and facilitating dispersion and absorption of coadministered agents (rHuPH20 Investigator's Brochure, 2018).

rHuPH20 is a glycosylated single chain protein with up to 447 amino acids, synthesized in CHO cells. As described above, rHuPH20 may be produced from a polynucleotide encoding amino acids 1-482 of SEQ ID NO: 843 (set forth in SEQ ID NO: 853), and post translational processing removes the 35 amino acid signal sequence, resulting in a polypeptide containing amino acids 36-482 of SEQ ID NO: 843 (set forth in SEQ ID NO: 845). As produced in the culture medium there is heterogeneity at the C-terminus such that the resulting rHuPH20 includes a mixture of species that can include any one or more of SEQ ID NOs: 845-850 in various abundance. Recombinant human hyaluronidase PH20 degrades HA under physiologic conditions and acts as a spreading factor in vivo. Therefore, when combined (co-mixed) or co-formulated with certain injectable drugs, rHUPH20 facilitates the absorption and dispersion of these drugs by temporarily reducing resistance to bulk fluid flow in the subcutaneous space. The permeability barrier in these tissues is restored to pre-injection levels within 24 to 48 hours after injection of rHuPH20.

Any suitable hyaluronidase (e.g., a recombinant human hyaluronidase) can be used in the methods, compositions, and combinations described herein, including, but not limited to, those described in U.S. Pat. No. 7,767,429 (e.g., SEQ ID NO:1), U.S. Pat. No. 7,846,431 (e.g., SEQ ID NO:1), U.S. Pat. No. 7,871,607 (e.g., SEQ ID NO:1), U.S. Pat. No. 8,105,586 (e.g., SEQ ID NO:1), U.S. Pat. No. 8,202,517 (e.g., SEQ ID NO:1), U.S. Pat. No. 8,257,699 (e.g., SEQ ID NO:1), U.S. Pat. No. 8,450,470 (e.g., SEQ ID NO:1), U.S. Pat. No. 8,431,124 (e.g., SEQ ID NO:1), U.S. Pat. No. 8,431,380 (e.g., SEQ ID NO:1), U.S. Pat. No. 8,580,252 (e.g., SEQ ID NO:1), U.S. Pat. No. 8,765,685 (e.g., SEQ ID NO:1), U.S. Pat. No. 8,772,246 (e.g., SEQ ID NO:1), U.S. Pat. No. 9,211,315 (e.g., SEQ ID NO:1), U.S. Pat. No. 9,562,223 (e.g., SEQ ID NO:1), U.S. Pat. No. 9,677,061 (e.g., SEQ ID NO:1), U.S. Pat. No. 9,677,062 (e.g., SEQ ID NO:1), U.S. Pat. No. 5,721,348 (e.g., SEQ ID NO:6), US 20210155913, US 20210363270, and US 20220289864, the contents of each of which are expressly incorporated herein by reference. The generation of such recombinant human hyaluronidases is described in U.S. Pat. Nos. 7,767,429; 7,871,607; and US20060104968, the contents of each of which are expressly incorporated herein by reference.

An exemplary recombinant human hyaluronidase is a soluble hyaluronidase provided as a composition designated rHuPH20, i.e., the active ingredient in the commercial product Hylenex® recombinant (hyaluronidase human injection), which is supplied as ENHANZE® drug product. ENHANZE® is a formulation containing rHuPH20 that can be admixed with an antibody prior to subcutaneous injection.

Hyaluronidases that have altered properties, such as increased stability and/or activity, have been produced and can also be used, including, but not limited to those described in U.S. Pat. Nos. 9,447,401, 10,865,400, and 11,041,149, the contents of each of which are expressly incorporated herein by reference. These patents provide about 7000 examples in which the effects of replacing each amino acid with 15 other amino acids on activity and stability were identified and described.

Other variants are known to those of skill in the art including those described, for example, in WO2020/022791 and WO2020197230A, the contents of each of which are expressly incorporated herein by reference. These variant polypeptides include replacements, insertions, and deletions, including one or more amino acid residues S343E, M345T, K349E, L353A, L354I, N356E, and 136 IT. Variants that contain such modifications and others are set forth in SEQ ID NOs: 60-115 from WO2020/022791. WO2021/150079 also provides variant polypeptides described as having increased stability.

In one embodiment, the recombinant human hyaluronidase includes a sequence of amino acids in any one of SEQ ID NOs: 843-852 and 854-909, or has at least 80%, 81%, 82%, 83%, 84%, 85%, 86%, 87%, 88%, 89%, 90%, 91%, 92%, 93%, 94%, 95%, 96%, 97%, 98%, or 99% sequence identity to a sequence of amino acids included in any one of SEQ ID NOs: 843-852 and 854-909 and retains hyaluronidase activity. In one embodiment, the recombinant human hyaluronidase comprises a sequence having at least 95%, 96%, 97%, 98%, or 99% to the amino acid sequence of SEQ ID NO: 843. In one embodiment, the recombinant human hyaluronidase comprises a sequence having at least 95%, 96%, 97%, 98%, or 99% to the amino acid sequence of SEQ ID NO: 845.

In another embodiment, the recombinant human hyaluronidase comprises the amino acid sequence set forth in SEQ ID NO:843. In another embodiment, the recombinant human hyaluronidase consists of the amino acid sequence set forth in SEQ ID NO:843. In another embodiment, the recombinant human hyaluronidase comprises the amino acid sequence set forth in SEQ ID NO:844. In another embodiment, the recombinant human hyaluronidase consists of the amino acid sequence set forth in SEQ ID NO:844. In another embodiment, the recombinant human hyaluronidase comprises the amino acid sequence set forth in SEQ ID NO:845. In another embodiment, the recombinant human hyaluronidase consists of the amino acid sequence set forth in SEQ ID NO:845. In another embodiment, the recombinant human hyaluronidase comprises the amino acid sequence set forth in SEQ ID NO:846. In another embodiment, the recombinant human hyaluronidase consists of the amino acid sequence set forth in SEQ ID NO:846. In another embodiment, the recombinant human hyaluronidase comprises the amino acid sequence set forth in SEQ ID NO:847. In another embodiment, the recombinant human hyaluronidase consists of the amino acid sequence set forth in SEQ ID NO:847. In another embodiment, the recombinant human hyaluronidase comprises the amino acid sequence set forth in SEQ ID NO:848. In another embodiment, the recombinant human hyaluronidase consists of the amino acid sequence set forth in SEQ ID NO:848. In another embodiment, the recombinant human hyaluronidase comprises the amino acid sequence set forth in SEQ ID NO:849. In another embodiment, the recombinant human hyaluronidase consists of the amino acid sequence set forth in SEQ ID NO:849. In another embodiment, the recombinant human hyaluronidase comprises the amino acid sequence set forth in SEQ ID NO:850. In another embodiment, the recombinant human hyaluronidase consists of the amino acid sequence set forth in SEQ ID NO:850. In another embodiment, the recombinant human hyaluronidase comprises the amino acid sequence set forth in SEQ ID NO:851. In another embodiment, the recombinant human hyaluronidase consists of the amino acid sequence set forth in SEQ ID NO:851. In another embodiment, the recombinant human hyaluronidase comprises the amino acid sequence set forth in SEQ ID NO:852. In another embodiment, the recombinant human hyaluronidase consists of the amino acid sequence set forth in SEQ ID NO:852. In another embodiment, the recombinant human hyaluronidase comprises the amino acid sequence set forth in SEQ ID NO:854. In another embodiment, the recombinant human hyaluronidase consists of the amino acid sequence set forth in SEQ ID NO:854. In another embodiment, the recombinant human hyaluronidase comprises the amino acid sequence set forth in SEQ ID NO:855. In another embodiment, the recombinant human hyaluronidase consists of the amino acid sequence set forth in SEQ ID NO:855. In another embodiment, the recombinant human hyaluronidase comprises the amino acid sequence set forth in SEQ ID NO:856. In another embodiment, the recombinant human hyaluronidase consists of the amino acid sequence set forth in SEQ ID NO:856. In another embodiment, the recombinant human hyaluronidase comprises the amino acid sequence set forth in SEQ ID NO:857. In another embodiment, the recombinant human hyaluronidase consists of the amino acid sequence set forth in SEQ ID NO:857. In another embodiment, the recombinant human hyaluronidase comprises the amino acid sequence set forth in SEQ ID NO:858. In another embodiment, the recombinant human hyaluronidase consists of the amino acid sequence set forth in SEQ ID NO:858. In another embodiment, the recombinant human hyaluronidase comprises the amino acid sequence set forth in SEQ ID NO:859. In another embodiment, the recombinant human hyaluronidase consists of the amino acid sequence set forth in SEQ ID NO:859. In another embodiment, the recombinant human hyaluronidase comprises the amino acid sequence set forth in SEQ ID NO:860. In another embodiment, the recombinant human hyaluronidase consists of the amino acid sequence set forth in SEQ ID NO:860. In another embodiment, the recombinant human hyaluronidase comprises the amino acid sequence set forth in SEQ ID NO:861. In another embodiment, the recombinant human hyaluronidase consists of the amino acid sequence set forth in SEQ ID NO:861. In another embodiment, the recombinant human hyaluronidase comprises the amino acid sequence set forth in SEQ ID NO:862. In another embodiment, the recombinant human hyaluronidase consists of the amino acid sequence set forth in SEQ ID NO:862. In another embodiment, the recombinant human hyaluronidase comprises the amino acid sequence set forth in SEQ ID NO:863. In another embodiment, the recombinant human hyaluronidase consists of the amino acid sequence set forth in SEQ ID NO:863. In another embodiment, the recombinant human hyaluronidase comprises the amino acid sequence set forth in SEQ ID NO:864. In another embodiment, the recombinant human hyaluronidase consists of the amino acid sequence set forth in SEQ ID NO:864. In another embodiment, the recombinant human hyaluronidase comprises the amino acid sequence set forth in SEQ ID NO:865. In another embodiment, the recombinant human hyaluronidase consists of the amino acid sequence set forth in SEQ ID NO:865. In another embodiment, the recombinant human hyaluronidase comprises the amino acid sequence set forth in SEQ ID NO:866. In another embodiment, the recombinant human hyaluronidase consists of the amino acid sequence set forth in SEQ ID NO:866. In another embodiment, the recombinant human hyaluronidase comprises the amino acid sequence set forth in SEQ ID NO:867. In another embodiment, the recombinant human hyaluronidase consists of the amino acid sequence set forth in SEQ ID NO:867. In another embodiment, the recombinant human hyaluronidase comprises the amino acid sequence set forth in SEQ ID NO:868. In another embodiment, the recombinant human hyaluronidase consists of the amino acid sequence set forth in SEQ ID NO:868. In another embodiment, the recombinant human hyaluronidase comprises the amino acid sequence set forth in SEQ ID NO:869. In another embodiment, the recombinant human hyaluronidase consists of the amino acid sequence set forth in SEQ ID NO:869. In another embodiment, the recombinant human hyaluronidase comprises the amino acid sequence set forth in SEQ ID NO:870. In another embodiment, the recombinant human hyaluronidase consists of the amino acid sequence set forth in SEQ ID NO:870. In another embodiment, the recombinant human hyaluronidase comprises the amino acid sequence set forth in SEQ ID NO:871. In another embodiment, the recombinant human hyaluronidase consists of the amino acid sequence set forth in SEQ ID NO:871. In another embodiment, the recombinant human hyaluronidase comprises the amino acid sequence set forth in SEQ ID NO:872. In another embodiment, the recombinant human hyaluronidase consists of the amino acid sequence set forth in SEQ ID NO:872. In another embodiment, the recombinant human hyaluronidase comprises the amino acid sequence set forth in SEQ ID NO:873. In another embodiment, the recombinant human hyaluronidase consists of the amino acid sequence set forth in SEQ ID NO:873. In another embodiment, the recombinant human hyaluronidase comprises the amino acid sequence set forth in SEQ ID NO:874. In another embodiment, the recombinant human hyaluronidase consists of the amino acid sequence set forth in SEQ ID NO:874. In another embodiment, the recombinant human hyaluronidase comprises the amino acid sequence set forth in SEQ ID NO:875. In another embodiment, the recombinant human hyaluronidase consists of the amino acid sequence set forth in SEQ ID NO:875. In another embodiment, the recombinant human hyaluronidase comprises the amino acid sequence set forth in SEQ ID NO:876. In another embodiment, the recombinant human hyaluronidase consists of the amino acid sequence set forth in SEQ ID NO:876. In another embodiment, the recombinant human hyaluronidase comprises the amino acid sequence set forth in SEQ ID NO:877. In another embodiment, the recombinant human hyaluronidase consists of the amino acid sequence set forth in SEQ ID NO:877. In another embodiment, the recombinant human hyaluronidase comprises the amino acid sequence set forth in SEQ ID NO:878. In another embodiment, the recombinant human hyaluronidase consists of the amino acid sequence set forth in SEQ ID NO:878. In another embodiment, the recombinant human hyaluronidase comprises the amino acid sequence set forth in SEQ ID NO:879. In another embodiment, the recombinant human hyaluronidase consists of the amino acid sequence set forth in SEQ ID NO:879. In another embodiment, the recombinant human hyaluronidase comprises the amino acid sequence set forth in SEQ ID NO:880. In another embodiment, the recombinant human hyaluronidase consists of the amino acid sequence set forth in SEQ ID NO:880. In another embodiment, the recombinant human hyaluronidase comprises the amino acid sequence set forth in SEQ ID NO:881. In another embodiment, the recombinant human hyaluronidase consists of the amino acid sequence set forth in SEQ ID NO:881. In another embodiment, the recombinant human hyaluronidase comprises the amino acid sequence set forth in SEQ ID NO:882. In another embodiment, the recombinant human hyaluronidase consists of the amino acid sequence set forth in SEQ ID NO:882. In another embodiment, the recombinant human hyaluronidase comprises the amino acid sequence set forth in SEQ ID NO:883. In another embodiment, the recombinant human hyaluronidase consists of the amino acid sequence set forth in SEQ ID NO:883. In another embodiment, the recombinant human hyaluronidase comprises the amino acid sequence set forth in SEQ ID NO:884. In another embodiment, the recombinant human hyaluronidase consists of the amino acid sequence set forth in SEQ ID NO:884. In another embodiment, the recombinant human hyaluronidase comprises the amino acid sequence set forth in SEQ ID NO:885. In another embodiment, the recombinant human hyaluronidase consists of the amino acid sequence set forth in SEQ ID NO:885. In another embodiment, the recombinant human hyaluronidase comprises the amino acid sequence set forth in SEQ ID NO:886. In another embodiment, the recombinant human hyaluronidase consists of the amino acid sequence set forth in SEQ ID NO:886. In another embodiment, the recombinant human hyaluronidase comprises the amino acid sequence set forth in SEQ ID NO:887. In another embodiment, the recombinant human hyaluronidase consists of the amino acid sequence set forth in SEQ ID NO:887. In another embodiment, the recombinant human hyaluronidase comprises the amino acid sequence set forth in SEQ ID NO:888. In another embodiment, the recombinant human hyaluronidase consists of the amino acid sequence set forth in SEQ ID NO:888. In another embodiment, the recombinant human hyaluronidase comprises the amino acid sequence set forth in SEQ ID NO:889. In another embodiment, the recombinant human hyaluronidase consists of the amino acid sequence set forth in SEQ ID NO:889. In another embodiment, the recombinant human hyaluronidase comprises the amino acid sequence set forth in SEQ ID NO:890. In another embodiment, the recombinant human hyaluronidase consists of the amino acid sequence set forth in SEQ ID NO:890. In another embodiment, the recombinant human hyaluronidase comprises the amino acid sequence set forth in SEQ ID NO:891. In another embodiment, the recombinant human hyaluronidase consists of the amino acid sequence set forth in SEQ ID NO:891. In another embodiment, the recombinant human hyaluronidase comprises the amino acid sequence set forth in SEQ ID NO:892. In another embodiment, the recombinant human hyaluronidase consists of the amino acid sequence set forth in SEQ ID NO:892. In another embodiment, the recombinant human hyaluronidase comprises the amino acid sequence set forth in SEQ ID NO:893. In another embodiment, the recombinant human hyaluronidase consists of the amino acid sequence set forth in SEQ ID NO:893. In another embodiment, the recombinant human hyaluronidase comprises the amino acid sequence set forth in SEQ ID NO:894. In another embodiment, the recombinant human hyaluronidase consists of the amino acid sequence set forth in SEQ ID NO:894. In another embodiment, the recombinant human hyaluronidase comprises the amino acid sequence set forth in SEQ ID NO:895. In another embodiment, the recombinant human hyaluronidase consists of the amino acid sequence set forth in SEQ ID NO:895. In another embodiment, the recombinant human hyaluronidase comprises the amino acid sequence set forth in SEQ ID NO:896. In another embodiment, the recombinant human hyaluronidase consists of the amino acid sequence set forth in SEQ ID NO:896. In another embodiment, the recombinant human hyaluronidase comprises the amino acid sequence set forth in SEQ ID NO:897. In another embodiment, the recombinant human hyaluronidase consists of the amino acid sequence set forth in SEQ ID NO:897. In another embodiment, the recombinant human hyaluronidase comprises the amino acid sequence set forth in SEQ ID NO:898. In another embodiment, the recombinant human hyaluronidase consists of the amino acid sequence set forth in SEQ ID NO:898. In another embodiment, the recombinant human hyaluronidase comprises the amino acid sequence set forth in SEQ ID NO:899. In another embodiment, the recombinant human hyaluronidase consists of the amino acid sequence set forth in SEQ ID NO:899. In another embodiment, the recombinant human hyaluronidase comprises the amino acid sequence set forth in SEQ ID NO:900. In another embodiment, the recombinant human hyaluronidase consists of the amino acid sequence set forth in SEQ ID NO:900. In another embodiment, the recombinant human hyaluronidase comprises the amino acid sequence set forth in SEQ ID NO:901. In another embodiment, the recombinant human hyaluronidase consists of the amino acid sequence set forth in SEQ ID NO:901. In another embodiment, the recombinant human hyaluronidase comprises the amino acid sequence set forth in SEQ ID NO:902. In another embodiment, the recombinant human hyaluronidase consists of the amino acid sequence set forth in SEQ ID NO:902. In another embodiment, the recombinant human hyaluronidase comprises the amino acid sequence set forth in SEQ ID NO:903. In another embodiment, the recombinant human hyaluronidase consists of the amino acid sequence set forth in SEQ ID NO:903. In another embodiment, the recombinant human hyaluronidase comprises the amino acid sequence set forth in SEQ ID NO:904. In another embodiment, the recombinant human hyaluronidase consists of the amino acid sequence set forth in SEQ ID NO:904. In another embodiment, the recombinant human hyaluronidase comprises the amino acid sequence set forth in SEQ ID NO:905. In another embodiment, the recombinant human hyaluronidase consists of the amino acid sequence set forth in SEQ ID NO:905. In another embodiment, the recombinant human hyaluronidase comprises the amino acid sequence set forth in SEQ ID NO:906. In another embodiment, the recombinant human hyaluronidase consists of the amino acid sequence set forth in SEQ ID NO:906. In another embodiment, the recombinant human hyaluronidase comprises the amino acid sequence set forth in SEQ ID NO:907. In another embodiment, the recombinant human hyaluronidase consists of the amino acid sequence set forth in SEQ ID NO:907. In another embodiment, the recombinant human hyaluronidase comprises the amino acid sequence set forth in SEQ ID NO:908. In another embodiment, the recombinant human hyaluronidase consists of the amino acid sequence set forth in SEQ ID NO:908. In another embodiment, the recombinant human hyaluronidase comprises the amino acid sequence set forth in SEQ ID NO:909. In another embodiment, the recombinant human hyaluronidase consists of the amino acid sequence set forth in SEQ ID NO:909.

### Anti-OX40L Antibodies

### Antibody Structure

The present application provides antibodies and antigen binding fragments thereof, and compositions and combinations comprising an antibody, or antigen binding fragment thereof, which binds OX40L as well as methods of use thereof. Exemplary anti-OX40L antibodies are described in PCT Publication No. WO2024227174, which is incorporated herein by reference in its entirety. In some embodiments, an anti-OX40L antibody, or antigen binding fragment thereof, described herein is selected from an anti-OX40L antibody described in PCT Publication No. WO2024227174.

The recognized immunoglobulin genes include the kappa, lambda, alpha, gamma, delta, epsilon and mu constant region genes, as well as the myriad immunoglobulin variable region genes. Light chains are classified as either kappa or lambda. The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

An exemplary immunoglobulin (antibody) structural unit is composed of two pairs of polypeptide chains, each pair having one “light” (about 25 kD) and one “heavy” chain (about 50-70 kD). The N-terminal domain of each chain defines a variable region of about 100 to 110 or more amino acids primarily responsible for antigen recognition. The terms variable light chain (VL) and variable heavy chain (VH) refer to these light and heavy chain domains respectively. The IgG1 heavy chain comprises of the VH, CH1, CH2 and CH3 domains respectively from the N to C-terminus. The light chain comprises of the VL and CL domains from N to C terminus. The IgG1 heavy chain comprises a hinge between the CH1 and CH2 domains. In certain embodiments, the immunoglobulin constructs comprise at least one immunoglobulin domain from IgG, IgM, IgA, IgD, or IgE connected to a therapeutic polypeptide. In some embodiments, the immunoglobulin domain found in an antibody provided herein, is from or derived from an immunoglobulin-based construct such as a diabody, or a nanobody. In certain embodiments, the immunoglobulin constructs described herein comprise at least one immunoglobulin domain from a heavy chain antibody such as a camelid antibody. In certain embodiments, the immunoglobulin constructs provided herein comprise at least one immunoglobulin domain from a mammalian antibody such as a bovine antibody, a human antibody, a camelid antibody, a mouse antibody or any chimeric antibody.

In some embodiments, the antibodies provided herein comprise a heavy chain. In one embodiment, the heavy chain is an IgA. In one embodiment, the heavy chain is an IgD. In one embodiment, the heavy chain is an IgE. In one embodiment, the heavy chain is an IgG. In one embodiment, the heavy chain is an IgM. In one embodiment, the heavy chain is an IgG1. In one embodiment, the heavy chain is an IgG2. In one embodiment, the heavy chain is an IgG3. In one embodiment, the heavy chain is an IgG4. In one embodiment, the heavy chain is an IgA1. In one embodiment, the heavy chain is an IgA2.

In some embodiments, an antibody is an IgG1 antibody. In some embodiments, an antibody is an IgG3 antibody. In some embodiments, an antibody is an IgG2 antibody. In some embodiments, an antibody is an IgG4 antibody.

Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen-binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The exact residue numbers which encompass a particular CDR will vary depending on the sequence and size of the CDR. Those skilled in the art can routinely determine which residues comprise a particular CDR given the variable region amino acid sequence of the antibody.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“aHo” numbering scheme); each of which is incorporated by reference in its entirety.

TABLE 2 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

CDRs may be assigned, for example, using antibody numbering software, such as Abnum, available at www.bioinf.org.uk/abs/abnum/, and described in Abhinandan and Martin, Immunology, 2008, 45:3832-3839, incorporated by reference in its entirety.

TABLE 2
Residues in CDRs according to Kabat and Chothia numbering schemes.
TABLE 1

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

*The C-terminus of CDR-H1, when numbered using the Kabat numbering convention, varies between H32 and H34, depending on the length of the CDR.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

One example of an antigen-binding domain is an antigen-binding domain formed by a VH-VL dimer of an antibody. Another example of an antigen-binding domain is an antigen-binding domain formed by diversification of certain loops from the tenth fibronectin type III domain of an Adnectin. An antigen-binding domain can include CDRs 1, 2, and 3 from a heavy chain in that order; and CDRs 1, 2, and 3 from a light chain in that order.

Epitopes frequently consist of surface-accessible amino acid residues and/or sugar side chains and may have specific three-dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to OX40L variants with different point-mutations, or to chimeric OX40L variants.

To screen for antibodies which bind to an epitope on a target antigen bound by an antibody of interest (e.g., OX40L), a routine cross-blocking assay such as that described in Antibodies, A Laboratory Manual, Cold Spring Harbor Laboratory, Ed Harlow and David Lane (1988), can be performed. Alternatively, or additionally, epitope mapping can be performed by methods known in the art.

Chimeric antibodies are antibodies in which a portion of the heavy and/or light chain is derived from a particular source or species, while the remainder of the heavy and/or light chain is derived from a different source or species.

Human antibodies are antibodies which possess an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

A humanized antibody has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies can be found in U.S. Pat. Nos. 6,054,297, 5,886,152 and 5,877,293. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

In some embodiments, an antibody can bind to two or more different epitopes. The two or more different epitopes may be epitopes on the same antigen (e.g., a single OX40L) or on different antigens (e.g., different OX40L molecules, or an OX40L molecule and a non-OX40L molecule). In some aspects, a multi-specific antibody binds two different epitopes (i.e., a “bispecific antibody”). In some aspects, a multi-specific antibody binds three different epitopes (i.e., a “trispecific antibody”).

Anti-OX40L antibodies can include those described herein such as the clones set forth in the drawings and/or tables. In some embodiments, the antibody comprises an alternative scaffold. In some embodiments, the antibody consists of an alternative scaffold. In some embodiments, the antibody consists essentially of an alternative scaffold. In some embodiments, the antibody comprises an antibody fragment. In some embodiments, the antibody consists of an antibody fragment. In some embodiments, the antibody consists essentially of an antibody fragment.

In some embodiments, the antibodies are monoclonal antibodies.

In some embodiments, the antibodies are polyclonal antibodies.

In some embodiments, the antibodies are produced by hybridomas. In other embodiments, the antibodies are produced by recombinant cells engineered to express the desired variable and constant domains.

In some embodiments, the antibodies may be single chain antibodies or other antibody derivatives retaining the antigen specificity and the lower hinge region or a variant thereof.

In some embodiments, the antibodies may be polyfunctional antibodies, recombinant antibodies, human antibodies, humanized antibodies, fragments or variants thereof. In particular embodiments, the antibody fragment or a derivative thereof is selected from a Fab fragment, a Fab′2 fragment, a CDR and ScFv.

In some embodiments, the antibodies are capable of forming an immune complex.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

TABLE 3
Sequences of OX40L Antibody Constructs - VH, VL, and Associated CDRs

| Construct Identifier | VH and Heavy chain CDRs | VL and Light chain CDRs |
|---|---|---|
| 114 | VH (SEQ ID NO: 419) | VL (SEQ ID NO: 489) |
| | CDR1: Kabat (SEQ ID NO: 1) | CDR1: Kabat (SEQ ID NO: 258) |
| | Chothia (SEQ ID NO: 22) | Chothia (SEQ ID NO: 286) |
| | IMGT (SEQ ID NO: 44) | IMGT (SEQ ID NO: 311) |
| | CDR2: Kabat (SEQ ID NO: 70) | CDR2: Kabat (SEQ ID NO: 335) |
| | Chothia (SEQ ID NO: 111) | Chothia (SEQ ID NO: 359) |
| | IMGT (SEQ ID NO: 142) | IMGT (SEQ ID NO: 359) |
| | CDR3: Kabat (SEQ ID NO: 175) | CDR3: Kabat (SEQ ID NO: 368) |
| | Chothia (SEQ ID NO: 203) | Chothia (SEQ ID NO: 394) |
| | IMGT (SEQ ID NO: 223) | IMGT (SEQ ID NO: 368) |
| 117 | VH (SEQ ID NO: 420) | VL (SEQ ID NO: 490) |
| | CDR1: Kabat (SEQ ID NO: 2) | CDR1: Kabat (SEQ ID NO: 259) |
| | Chothia (SEQ ID NO: 23) | Chothia (SEQ ID NO: 287) |
| | IMGT (SEQ ID NO: 45) | IMGT (SEQ ID NO: 312) |
| | CDR2: Kabat (SEQ ID NO: 70) | CDR2: Kabat (SEQ ID NO: 336) |
| | Chothia (SEQ ID NO: 111) | Chothia (SEQ ID NO: 360) |
| | IMGT (SEQ ID NO: 142) | IMGT (SEQ ID NO: 360) |
| | CDR3: Kabat (SEQ ID NO: 176) | CDR3: Kabat (SEQ ID NO: 369) |
| | Chothia (SEQ ID NO: 204) | Chothia (SEQ ID NO: 395) |
| | IMGT (SEQ ID NO: 224) | IMGT (SEQ ID NO: 369) |
| 118 | VH (SEQ ID NO: 421) | VL (SEQ ID NO: 491) |
| | CDR1: Kabat (SEQ ID NO: 3) | CDR1: Kabat (SEQ ID NO: 260) |
| | Chothia (SEQ ID NO: 24) | Chothia (SEQ ID NO: 288) |
| | IMGT (SEQ ID NO: 46) | IMGT (SEQ ID NO: 313) |
| | CDR2: Kabat (SEQ ID NO: 71) | CDR2: Kabat (SEQ ID NO: 337) |
| | Chothia (SEQ ID NO: 112) | Chothia (SEQ ID NO: 361) |
| | IMGT (SEQ ID NO: 143) | IMGT (SEQ ID NO: 361) |
| | CDR3: Kabat (SEQ ID NO: 177) | CDR3: Kabat (SEQ ID NO: 370) |
| | Chothia (SEQ ID NO: 205) | Chothia (SEQ ID NO: 396) |
| | IMGT (SEQ ID NO: 225) | IMGT (SEQ ID NO: 370) |
| 122 | VH (SEQ ID NO: 422) | VL (SEQ ID NO: 492) |
| | CDR1: Kabat (SEQ ID NO: 4) | CDR1: Kabat (SEQ ID NO: 261) |
| | Chothia (SEQ ID NO: 22) | Chothia (SEQ ID NO: 289) |
| | IMGT (SEQ ID NO: 47) | IMGT (SEQ ID NO: 314) |
| | CDR2: Kabat (SEQ ID NO: 72) | CDR2: Kabat (SEQ ID NO: 337) |
| | Chothia (SEQ ID NO: 113) | Chothia (SEQ ID NO: 361) |
| | IMGT (SEQ ID NO: 144) | IMGT (SEQ ID NO: 361) |
| | CDR3: Kabat (SEQ ID NO: 178) | CDR3: Kabat (SEQ ID NO: 371) |
| | Chothia (SEQ ID NO: 206) | Chothia (SEQ ID NO: 397) |
| | IMGT (SEQ ID NO: 226) | IMGT (SEQ ID NO: 371) |
| 127 | VH (SEQ ID NO: 423) | VL (SEQ ID NO: 493) |
| | CDR1: Kabat (SEQ ID NO: 5) | CDR1: Kabat (SEQ ID NO: 262) |
| | Chothia (SEQ ID NO: 840) | Chothia (SEQ ID NO: 290) |
| | IMGT (SEQ ID NO: 48) | IMGT (SEQ ID NO: 315) |
| | CDR2: Kabat (SEQ ID NO: 73) | CDR2: Kabat (SEQ ID NO: 338) |
| | Chothia (SEQ ID NO: 114) | Chothia (SEQ ID NO: 362) |
| | IMGT (SEQ ID NO: 145) | IMGT (SEQ ID NO: 362) |
| | CDR3: Kabat (SEQ ID NO: 179) | CDR3: Kabat (SEQ ID NO: 372) |
| | Chothia (SEQ ID NO: 207) | Chothia (SEQ ID NO: 398) |
| | IMGT (SEQ ID NO: 227) | IMGT (SEQ ID NO: 372) |
| 128 | VH (SEQ ID NO: 424) | VL (SEQ ID NO: 494) |
| | CDR1: Kabat (SEQ ID NO: 6) | CDR1: Kabat (SEQ ID NO: 263) |
| | Chothia (SEQ ID NO: 24) | Chothia (SEQ ID NO: 291) |
| | IMGT (SEQ ID NO: 49) | IMGT (SEQ ID NO: 316) |
| | CDR2: Kabat (SEQ ID NO: 74) | CDR2: Kabat (SEQ ID NO: 339) |
| | Chothia (SEQ ID NO: 115) | Chothia (SEQ ID NO: 361) |
| | IMGT (SEQ ID NO: 146) | IMGT (SEQ ID NO: 361) |
| | CDR3: Kabat (SEQ ID NO: 180) | CDR3: Kabat (SEQ ID NO: 373) |
| | Chothia (SEQ ID NO: 208) | Chothia (SEQ ID NO: 399) |
| | IMGT (SEQ ID NO: 228) | IMGT (SEQ ID NO: 373) |
| 130 | VH (SEQ ID NO: 425) | VL (SEQ ID NO: 495) |
| | CDR1: Kabat (SEQ ID NO: 7) | CDR1: Kabat (SEQ ID NO: 264) |
| | Chothia (SEQ ID NO: 25) | Chothia (SEQ ID NO: 292) |
| | IMGT (SEQ ID NO: 50) | IMGT (SEQ ID NO: 317) |
| | CDR2: Kabat (SEQ ID NO: 75) | CDR2: Kabat (SEQ ID NO: 340) |
| | Chothia (SEQ ID NO: 116) | Chothia (SEQ ID NO: 363) |
| | IMGT (SEQ ID NO: 147) | IMGT (SEQ ID NO: 363) |
| | CDR3: Kabat (SEQ ID NO: 181) | CDR3: Kabat (SEQ ID NO: 374) |
| | Chothia (SEQ ID NO: 209) | Chothia (SEQ ID NO: 400) |
| | IMGT (SEQ ID NO: 229) | IMGT (SEQ ID NO: 374) |
| 327 | VH (SEQ ID NO: 426) | VL (SEQ ID NO: 496) |
| | CDR1: Kabat (SEQ ID NO: 8) | CDR1: Kabat (SEQ ID NO: 265) |
| | Chothia (SEQ ID NO: 26) | Chothia (SEQ ID NO: 293) |
| | IMGT (SEQ ID NO: 51) | IMGT (SEQ ID NO: 318) |
| | CDR2: Kabat (SEQ ID NO: 76) | CDR2: Kabat (SEQ ID NO: 341) |
| | Chothia (SEQ ID NO: 117) | Chothia (SEQ ID NO: 361) |
| | IMGT (SEQ ID NO: 148) | IMGT (SEQ ID NO: 361) |
| | CDR3: Kabat (SEQ ID NO: 182) | CDR3: Kabat (SEQ ID NO: 375) |
| | Chothia (SEQ ID NO: 210) | Chothia (SEQ ID NO: 401) |
| | IMGT (SEQ ID NO: 230) | IMGT (SEQ ID NO: 375) |
| 329 | VH (SEQ ID NO: 427) | VL (SEQ ID NO: 497) |
| | CDR1: Kabat (SEQ ID NO: 8) | CDR1: Kabat (SEQ ID NO: 265) |
| | Chothia (SEQ ID NO: 26) | Chothia (SEQ ID NO: 293) |
| | IMGT (SEQ ID NO: 51) | IMGT (SEQ ID NO: 318) |
| | CDR2: Kabat (SEQ ID NO: 76) | CDR2: Kabat (SEQ ID NO: 341) |
| | Chothia (SEQ ID NO: 117) | Chothia (SEQ ID NO: 361) |
| | IMGT (SEQ ID NO: 148) | IMGT (SEQ ID NO: 361) |
| | CDR3: Kabat (SEQ ID NO: 183) | CDR3: Kabat (SEQ ID NO: 376) |
| | Chothia (SEQ ID NO: 211) | Chothia (SEQ ID NO: 402) |
| | IMGT (SEQ ID NO: 231) | IMGT (SEQ ID NO: 376) |
| 330 | VH (SEQ ID NO: 427) | VL (SEQ ID NO: 498) |
| | CDR1: Kabat (SEQ ID NO: 8) | CDR1: Kabat (SEQ ID NO: 265) |
| | Chothia (SEQ ID NO: 26) | Chothia (SEQ ID NO: 293) |
| | IMGT (SEQ ID NO: 51) | IMGT (SEQ ID NO: 318) |
| | CDR2: Kabat (SEQ ID NO: 76) | CDR2: Kabat (SEQ ID NO: 341) |
| | Chothia (SEQ ID NO: 117) | Chothia (SEQ ID NO: 361) |
| | IMGT (SEQ ID NO: 148) | IMGT (SEQ ID NO: 361) |
| | CDR3: Kabat (SEQ ID NO: 183) | CDR3: Kabat (SEQ ID NO: 377) |
| | Chothia (SEQ ID NO: 211) | Chothia (SEQ ID NO: 403) |
| | IMGT (SEQ ID NO: 231) | IMGT (SEQ ID NO: 377) |
| 331 | VH (SEQ ID NO: 428) | VL (SEQ ID NO: 497) |
| | CDR1: Kabat (SEQ ID NO: 8) | CDR1: Kabat (SEQ ID NO: 265) |
| | Chothia (SEQ ID NO: 26) | Chothia (SEQ ID NO: 293) |
| | IMGT (SEQ ID NO: 51) | IMGT (SEQ ID NO: 318) |
| | CDR2: Kabat (SEQ ID NO: 76) | CDR2: Kabat (SEQ ID NO: 341) |
| | Chothia (SEQ ID NO: 117) | Chothia (SEQ ID NO: 361) |
| | IMGT (SEQ ID NO: 148) | IMGT (SEQ ID NO: 361) |
| | CDR3: Kabat (SEQ ID NO: 184) | CDR3: Kabat (SEQ ID NO: 376) |
| | Chothia (SEQ ID NO: 212) | Chothia (SEQ ID NO: 402) |
| | IMGT (SEQ ID NO: 232) | IMGT (SEQ ID NO: 376) |
| 332 | VH (SEQ ID NO: 428) | VL (SEQ ID NO: 498) |
| | CDR1: Kabat (SEQ ID NO: 8) | CDR1: Kabat (SEQ ID NO: 265) |
| | Chothia (SEQ ID NO: 26) | Chothia (SEQ ID NO: 293) |
| | IMGT (SEQ ID NO: 51) | IMGT (SEQ ID NO: 318) |
| | CDR2: Kabat (SEQ ID NO: 76) | CDR2: Kabat (SEQ ID NO: 341) |
| | Chothia (SEQ ID NO: 117) | Chothia (SEQ ID NO: 361) |
| | IMGT (SEQ ID NO: 148) | IMGT (SEQ ID NO: 361) |
| | CDR3: Kabat (SEQ ID NO: 184) | CDR3: Kabat (SEQ ID NO: 377) |
| | Chothia (SEQ ID NO: 212) | Chothia (SEQ ID NO: 403) |
| | IMGT (SEQ ID NO: 232) | IMGT (SEQ ID NO: 377) |
| 333 | VH (SEQ ID NO: 429) | VL (SEQ ID NO: 497) |
| | CDR1: Kabat (SEQ ID NO: 8) | CDR1: Kabat (SEQ ID NO: 265) |
| | Chothia (SEQ ID NO: 26) | Chothia (SEQ ID NO: 293) |
| | IMGT (SEQ ID NO: 51) | IMGT (SEQ ID NO: 318) |
| | CDR2: Kabat (SEQ ID NO: 76) | CDR2: Kabat (SEQ ID NO: 341) |
| | Chothia (SEQ ID NO: 117) | Chothia (SEQ ID NO: 361) |
| | IMGT (SEQ ID NO: 148) | IMGT (SEQ ID NO: 361) |
| | CDR3: Kabat (SEQ ID NO: 185) | CDR3: Kabat (SEQ ID NO: 376) |
| | Chothia (SEQ ID NO: 213) | Chothia (SEQ ID NO: 402) |
| | IMGT (SEQ ID NO: 233) | IMGT (SEQ ID NO: 376) |
| 334 | VH (SEQ ID NO: 429) | VL (SEQ ID NO: 498) |
| | CDR1: Kabat (SEQ ID NO: 8) | CDR1: Kabat (SEQ ID NO: 265) |
| | Chothia (SEQ ID NO: 26) | Chothia (SEQ ID NO: 293) |
| | IMGT (SEQ ID NO: 51) | IMGT (SEQ ID NO: 318) |
| | CDR2: Kabat (SEQ ID NO: 76) | CDR2: Kabat (SEQ ID NO: 341) |
| | Chothia (SEQ ID NO: 117) | Chothia (SEQ ID NO: 361) |
| | IMGT (SEQ ID NO: 148) | IMGT (SEQ ID NO: 361) |
| | CDR3: Kabat (SEQ ID NO: 185) | CDR3: Kabat (SEQ ID NO: 377) |
| | Chothia (SEQ ID NO: 213) | Chothia (SEQ ID NO: 403) |
| | IMGT (SEQ ID NO: 233) | IMGT (SEQ ID NO: 377) |
| 501 | VH (SEQ ID NO: 430) | VL (SEQ ID NO: 499) |
| | CDR1: Kabat (SEQ ID NO: 9) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 77) | CDR2: Kabat (SEQ ID NO: 342) |
| | Chothia (SEQ ID NO: 118) | Chothia (SEQ ID NO: 364) |
| | IMGT (SEQ ID NO: 149) | IMGT (SEQ ID NO: 364) |
| | CDR3: Kabat (SEQ ID NO: 175) | CDR3: Kabat (SEQ ID NO: 368) |
| | Chothia (SEQ ID NO: 203) | Chothia (SEQ ID NO: 394) |
| | IMGT (SEQ ID NO: 234) | IMGT (SEQ ID NO: 368) |
| 502 | VH (SEQ ID NO: 431) | VL (SEQ ID NO: 500) |
| | CDR1: Kabat (SEQ ID NO: 1) | CDR1: Kabat (SEQ ID NO: 266) |
| | Chothia (SEQ ID NO: 28) | Chothia (SEQ ID NO: 286) |
| | IMGT (SEQ ID NO: 53) | IMGT (SEQ ID NO: 311) |
| | CDR2: Kabat (SEQ ID NO: 78) | CDR2: Kabat (SEQ ID NO: 343) |
| | Chothia (SEQ ID NO: 119) | Chothia (SEQ ID NO: 365) |
| | IMGT (SEQ ID NO: 150) | IMGT (SEQ ID NO: 365) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 223) | IMGT (SEQ ID NO: 378) |
| 503 | VH (SEQ ID NO: 432) | VL (SEQ ID NO: 501) |
| | CDR1: Kabat (SEQ ID NO: 10) | CDR1: Kabat (SEQ ID NO: 267) |
| | Chothia (SEQ ID NO: 27) | Chothia (SEQ ID NO: 294) |
| | IMGT (SEQ ID NO: 52) | IMGT (SEQ ID NO: 319) |
| | CDR2: Kabat (SEQ ID NO: 79) | CDR2: Kabat (SEQ ID NO: 344) |
| | Chothia (SEQ ID NO: 119) | Chothia (SEQ ID NO: 365) |
| | IMGT (SEQ ID NO: 150) | IMGT (SEQ ID NO: 365) |
| | CDR3: Kabat (SEQ ID NO: 186) | CDR3: Kabat (SEQ ID NO: 378) |
| | Chothia (SEQ ID NO: 203) | Chothia (SEQ ID NO: 404) |
| | IMGT (SEQ ID NO: 235) | IMGT (SEQ ID NO: 378) |
| 504 | VH (SEQ ID NO: 433) | VL (SEQ ID NO: 502) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 80) | CDR2: Kabat (SEQ ID NO: 343) |
| | Chothia (SEQ ID NO: 120) | Chothia (SEQ ID NO: 365) |
| | IMGT (SEQ ID NO: 151) | IMGT (SEQ ID NO: 365) |
| | CDR3: Kabat (SEQ ID NO: 186) | CDR3: Kabat (SEQ ID NO: 378) |
| | Chothia (SEQ ID NO: 203) | Chothia (SEQ ID NO: 404) |
| | IMGT (SEQ ID NO: 235) | IMGT (SEQ ID NO: 378) |
| 505 | VH (SEQ ID NO: 434) | VL (SEQ ID NO: 502) |
| | CDR1: Kabat (SEQ ID NO: 11) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 79) | CDR2: Kabat (SEQ ID NO: 343) |
| | Chothia (SEQ ID NO: 119) | Chothia (SEQ ID NO: 365) |
| | IMGT (SEQ ID NO: 150) | IMGT (SEQ ID NO: 365) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 223) | IMGT (SEQ ID NO: 378) |
| 506 | VH (SEQ ID NO: 435) | VL (SEQ ID NO: 502) |
| | CDR1: Kabat (SEQ ID NO: 11) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 81) | CDR2: Kabat (SEQ ID NO: 343) |
| | Chothia (SEQ ID NO: 121) | Chothia (SEQ ID NO: 365) |
| | IMGT (SEQ ID NO: 152) | IMGT (SEQ ID NO: 365) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 223) | IMGT (SEQ ID NO: 378) |
| 507 | VH (SEQ ID NO: 436) | VL (SEQ ID NO: 502) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 80) | CDR2: Kabat (SEQ ID NO: 343) |
| | Chothia (SEQ ID NO: 120) | Chothia (SEQ ID NO: 365) |
| | IMGT (SEQ ID NO: 151) | IMGT (SEQ ID NO: 365) |
| | CDR3: Kabat (SEQ ID NO: 187) | CDR3: Kabat (SEQ ID NO: 378) |
| | Chothia (SEQ ID NO: 203) | Chothia (SEQ ID NO: 404) |
| | IMGT (SEQ ID NO: 236) | IMGT (SEQ ID NO: 378) |
| 508 | VH (SEQ ID NO: 437) | VL (SEQ ID NO: 502) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 79) | CDR2: Kabat (SEQ ID NO: 343) |
| | Chothia (SEQ ID NO: 119) | Chothia (SEQ ID NO: 365) |
| | IMGT (SEQ ID NO: 150) | IMGT (SEQ ID NO: 365) |
| | CDR3: Kabat (SEQ ID NO: 187) | CDR3: Kabat (SEQ ID NO: 378) |
| | Chothia (SEQ ID NO: 203) | Chothia (SEQ ID NO: 404) |
| | IMGT (SEQ ID NO: 236) | IMGT (SEQ ID NO: 378) |
| 509 | VH (SEQ ID NO: 438) | VL (SEQ ID NO: 503) |
| | CDR1: Kabat (SEQ ID NO: 10) | CDR1: Kabat (SEQ ID NO: 268) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 70) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 188) | CDR3: Kabat (SEQ ID NO: 368) |
| | Chothia (SEQ ID NO: 203) | Chothia (SEQ ID NO: 394) |
| | IMGT (SEQ ID NO: 237) | IMGT (SEQ ID NO: 368) |
| 510 | VH (SEQ ID NO: 439) | VL (SEQ ID NO: 502) |
| | CDR1: Kabat (SEQ ID NO: 12) | CDR1: Kabat (SEQ ID NO: 260) |
| | Chothia (SEQ ID NO: 29) | Chothia (SEQ ID NO: 288) |
| | IMGT (SEQ ID NO: 54) | IMGT (SEQ ID NO: 313) |
| | CDR2: Kabat (SEQ ID NO: 70) | CDR2: Kabat (SEQ ID NO: 343) |
| | Chothia (SEQ ID NO: 111) | Chothia (SEQ ID NO: 365) |
| | IMGT (SEQ ID NO: 142) | IMGT (SEQ ID NO: 365) |
| | CDR3: Kabat (SEQ ID NO: 188) | CDR3: Kabat (SEQ ID NO: 378) |
| | Chothia (SEQ ID NO: 203) | Chothia (SEQ ID NO: 404) |
| | IMGT (SEQ ID NO: 237) | IMGT (SEQ ID NO: 378) |
| 511 | VH (SEQ ID NO: 440) | VL (SEQ ID NO: 504) |
| | CDR1: Kabat (SEQ ID NO: 10) | CDR1: Kabat (SEQ ID NO: 269) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 82) | CDR2: Kabat (SEQ ID NO: 345) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 189) | CDR3: Kabat (SEQ ID NO: 378) |
| | Chothia (SEQ ID NO: 214) | Chothia (SEQ ID NO: 404) |
| | IMGT (SEQ ID NO: 238) | IMGT (SEQ ID NO: 378) |
| 512 | VH (SEQ ID NO: 441) | VL (SEQ ID NO: 505) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 83) | CDR2: Kabat (SEQ ID NO: 346) |
| | Chothia (SEQ ID NO: 122) | Chothia (SEQ ID NO: 361) |
| | IMGT (SEQ ID NO: 153) | IMGT (SEQ ID NO: 361) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 234) | IMGT (SEQ ID NO: 378) |
| 513 | VH (SEQ ID NO: 442) | VL (SEQ ID NO: 506) |
| | CDR1: Kabat (SEQ ID NO: 13) | CDR1: Kabat (SEQ ID NO: 260) |
| | Chothia (SEQ ID NO: 30) | Chothia (SEQ ID NO: 288) |
| | IMGT (SEQ ID NO: 55) | IMGT (SEQ ID NO: 313) |
| | CDR2: Kabat (SEQ ID NO: 84) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 188) | CDR3: Kabat (SEQ ID NO: 378) |
| | Chothia (SEQ ID NO: 203) | Chothia (SEQ ID NO: 404) |
| | IMGT (SEQ ID NO: 239) | IMGT (SEQ ID NO: 378) |
| 514 | VH (SEQ ID NO: 443) | VL (SEQ ID NO: 507) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 70) | CDR2: Kabat (SEQ ID NO: 347) |
| | Chothia (SEQ ID NO: 111) | Chothia (SEQ ID NO: 365) |
| | IMGT (SEQ ID NO: 142) | IMGT (SEQ ID NO: 365) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 234) | IMGT (SEQ ID NO: 378) |
| 515 | VH (SEQ ID NO: 443) | VL (SEQ ID NO: 508) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 70) | CDR2: Kabat (SEQ ID NO: 345) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 234) | IMGT (SEQ ID NO: 378) |
| 516 | VH (SEQ ID NO: 444) | VL (SEQ ID NO: 506) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 85) | CDR2: Kabat (SEQ ID NO: 337) |
| | Chothia (SEQ ID NO: 123) | Chothia (SEQ ID NO: 361) |
| | IMGT (SEQ ID NO: 154) | IMGT (SEQ ID NO: 361) |
| | CDR3: Kabat (SEQ ID NO: 188) | CDR3: Kabat (SEQ ID NO: 378) |
| | Chothia (SEQ ID NO: 203) | Chothia (SEQ ID NO: 404) |
| | IMGT (SEQ ID NO: 239) | IMGT (SEQ ID NO: 378) |
| 517 | VH (SEQ ID NO: 445) | VL (SEQ ID NO: 509) |
| | CDR1: Kabat (SEQ ID NO: 10) | CDR1: Kabat (SEQ ID NO: 270) |
| | Chothia (SEQ ID NO: 27) | Chothia (SEQ ID NO: 295) |
| | IMGT (SEQ ID NO: 52) | IMGT (SEQ ID NO: 313) |
| | CDR2: Kabat (SEQ ID NO: 70) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 223) | IMGT (SEQ ID NO: 378) |
| 518 | VH (SEQ ID NO: 446) | VL (SEQ ID NO: 510) |
| | CDR1: Kabat (SEQ ID NO: 1) | CDR1: Kabat (SEQ ID NO: 260) |
| | Chothia (SEQ ID NO: 31) | Chothia (SEQ ID NO: 288) |
| | IMGT (SEQ ID NO: 56) | IMGT (SEQ ID NO: 313) |
| | CDR2: Kabat (SEQ ID NO: 70) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 175) | CDR3: Kabat (SEQ ID NO: 379) |
| | Chothia (SEQ ID NO: 203) | Chothia (SEQ ID NO: 405) |
| | IMGT (SEQ ID NO: 223) | IMGT (SEQ ID NO: 379) |
| 519 | VH (SEQ ID NO: 447) | VL (SEQ ID NO: 506) |
| | CDR1: Kabat (SEQ ID NO: 1) | CDR1: Kabat (SEQ ID NO: 260) |
| | Chothia (SEQ ID NO: 32) | Chothia (SEQ ID NO: 288) |
| | IMGT (SEQ ID NO: 57) | IMGT (SEQ ID NO: 313) |
| | CDR2: Kabat (SEQ ID NO: 86) | CDR2: Kabat (SEQ ID NO: 337) |
| | Chothia (SEQ ID NO: 124) | Chothia (SEQ ID NO: 361) |
| | IMGT (SEQ ID NO: 155) | IMGT (SEQ ID NO: 361) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 223) | IMGT (SEQ ID NO: 378) |
| 520 | VH (SEQ ID NO: 445) | VL (SEQ ID NO: 511) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 70) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 175) | CDR3: Kabat (SEQ ID NO: 380) |
| | Chothia (SEQ ID NO: 203) | Chothia (SEQ ID NO: 406) |
| | IMGT (SEQ ID NO: 223) | IMGT (SEQ ID NO: 380) |
| 521 | VH (SEQ ID NO: 448) | VL (SEQ ID NO: 506) |
| | CDR1: Kabat (SEQ ID NO: 14) | CDR1: Kabat (SEQ ID NO: 260) |
| | Chothia (SEQ ID NO: 33) | Chothia (SEQ ID NO: 288) |
| | IMGT (SEQ ID NO: 58) | IMGT (SEQ ID NO: 313) |
| | CDR2: Kabat (SEQ ID NO: 70) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 223) | IMGT (SEQ ID NO: 378) |
| 522 | VH (SEQ ID NO: 449) | VL (SEQ ID NO: 502) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 87) | CDR2: Kabat (SEQ ID NO: 343) |
| | Chothia (SEQ ID NO: 111) | Chothia (SEQ ID NO: 365) |
| | IMGT (SEQ ID NO: 142) | IMGT (SEQ ID NO: 365) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 223) | IMGT (SEQ ID NO: 378) |
| 523 | VH (SEQ ID NO: 449) | VL (SEQ ID NO: 506) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 87) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 223) | IMGT (SEQ ID NO: 378) |
| 524 | VH (SEQ ID NO: 450) | VL (SEQ ID NO: 506) |
| | CDR1: Kabat (SEQ ID NO: 13) | CDR1: Kabat (SEQ ID NO: 260) |
| | Chothia (SEQ ID NO: 30) | Chothia (SEQ ID NO: 288) |
| | IMGT (SEQ ID NO: 55) | IMGT (SEQ ID NO: 313) |
| | CDR2: Kabat (SEQ ID NO: 87) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 223) | IMGT (SEQ ID NO: 378) |
| 525 | VH (SEQ ID NO: 445) | VL (SEQ ID NO: 512) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 70) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 175) | CDR3: Kabat (SEQ ID NO: 368) |
| | Chothia (SEQ ID NO: 203) | Chothia (SEQ ID NO: 394) |
| | IMGT (SEQ ID NO: 223) | IMGT (SEQ ID NO: 368) |
| 526 | VH (SEQ ID NO: 449) | VL (SEQ ID NO: 513) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 87) | CDR2: Kabat (SEQ ID NO: 348) |
| | Chothia (SEQ ID NO: 111) | Chothia (SEQ ID NO: 366) |
| | IMGT (SEQ ID NO: 142) | IMGT (SEQ ID NO: 366) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 223) | IMGT (SEQ ID NO: 378) |
| 527 | VH (SEQ ID NO: 445) | VL (SEQ ID NO: 514) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 70) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 175) | CDR3: Kabat (SEQ ID NO: 381) |
| | Chothia (SEQ ID NO: 203) | Chothia (SEQ ID NO: 405) |
| | IMGT (SEQ ID NO: 223) | IMGT (SEQ ID NO: 381) |
| 528 | VH (SEQ ID NO: 445) | VL (SEQ ID NO: 515) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 70) | CDR2: Kabat (SEQ ID NO: 335) |
| | Chothia (SEQ ID NO: 111) | Chothia (SEQ ID NO: 359) |
| | IMGT (SEQ ID NO: 142) | IMGT (SEQ ID NO: 359) |
| | CDR3: Kabat (SEQ ID NO: 175) | CDR3: Kabat (SEQ ID NO: 368) |
| | Chothia (SEQ ID NO: 203) | Chothia (SEQ ID NO: 394) |
| | IMGT (SEQ ID NO: 223) | IMGT (SEQ ID NO: 368) |
| 529 | VH (SEQ ID NO: 451) | VL (SEQ ID NO: 514) |
| | CDR1: Kabat (SEQ ID NO: 1) | CDR1: Kabat (SEQ ID NO: 260) |
| | Chothia (SEQ ID NO: 22) | Chothia (SEQ ID NO: 288) |
| | IMGT (SEQ ID NO: 44) | IMGT (SEQ ID NO: 313) |
| | CDR2: Kabat (SEQ ID NO: 86) | CDR2: Kabat (SEQ ID NO: 337) |
| | Chothia (SEQ ID NO: 124) | Chothia (SEQ ID NO: 361) |
| | IMGT (SEQ ID NO: 155) | IMGT (SEQ ID NO: 361) |
| | CDR3: Kabat (SEQ ID NO: 175) | CDR3: Kabat (SEQ ID NO: 381) |
| | Chothia (SEQ ID NO: 203) | Chothia (SEQ ID NO: 405) |
| | IMGT (SEQ ID NO: 223) | IMGT (SEQ ID NO: 381) |
| 530 | VH (SEQ ID NO: 451) | VL (SEQ ID NO: 506) |
| | CDR1: Kabat (SEQ ID NO: 1) | CDR1: Kabat (SEQ ID NO: 260) |
| | Chothia (SEQ ID NO: 22) | Chothia (SEQ ID NO: 288) |
| | IMGT (SEQ ID NO: 44) | IMGT (SEQ ID NO: 313) |
| | CDR2: Kabat (SEQ ID NO: 86) | CDR2: Kabat (SEQ ID NO: 337) |
| | Chothia (SEQ ID NO: 124) | Chothia (SEQ ID NO: 361) |
| | IMGT (SEQ ID NO: 155) | IMGT (SEQ ID NO: 361) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 223) | IMGT (SEQ ID NO: 378) |
| 531 | VH (SEQ ID NO: 452) | VL (SEQ ID NO: 506) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 70) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 188) | CDR3: Kabat (SEQ ID NO: 378) |
| | Chothia (SEQ ID NO: 203) | Chothia (SEQ ID NO: 404) |
| | IMGT (SEQ ID NO: 239) | IMGT (SEQ ID NO: 378) |
| 532 | VH (SEQ ID NO: 438) | VL (SEQ ID NO: 506) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 70) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 188) | CDR3: Kabat (SEQ ID NO: 378) |
| | Chothia (SEQ ID NO: 203) | Chothia (SEQ ID NO: 404) |
| | IMGT (SEQ ID NO: 237) | IMGT (SEQ ID NO: 378) |
| 533 | VH (SEQ ID NO: 453) | VL (SEQ ID NO: 512) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 78) | CDR2: Kabat (SEQ ID NO: 337) |
| | Chothia (SEQ ID NO: 119) | Chothia (SEQ ID NO: 361) |
| | IMGT (SEQ ID NO: 150) | IMGT (SEQ ID NO: 361) |
| | CDR3: Kabat (SEQ ID NO: 188) | CDR3: Kabat (SEQ ID NO: 368) |
| | Chothia (SEQ ID NO: 203) | Chothia (SEQ ID NO: 394) |
| | IMGT (SEQ ID NO: 237) | IMGT (SEQ ID NO: 368) |
| 534 | VH (SEQ ID NO: 454) | VL (SEQ ID NO: 490) |
| | CDR1: Kabat (SEQ ID NO: 2) | CDR1: Kabat (SEQ ID NO: 259) |
| | Chothia (SEQ ID NO: 23) | Chothia (SEQ ID NO: 287) |
| | IMGT (SEQ ID NO: 45) | IMGT (SEQ ID NO: 312) |
| | CDR2: Kabat (SEQ ID NO: 70) | CDR2: Kabat (SEQ ID NO: 336) |
| | Chothia (SEQ ID NO: 111) | Chothia (SEQ ID NO: 360) |
| | IMGT (SEQ ID NO: 142) | IMGT (SEQ ID NO: 360) |
| | CDR3: Kabat (SEQ ID NO: 176) | CDR3: Kabat (SEQ ID NO: 369) |
| | Chothia (SEQ ID NO: 204) | Chothia (SEQ ID NO: 395) |
| | IMGT (SEQ ID NO: 224) | IMGT (SEQ ID NO: 369) |
| 535 | VH (SEQ ID NO: 455) | VL (SEQ ID NO: 516) |
| | CDR1: Kabat (SEQ ID NO: 15) | CDR1: Kabat (SEQ ID NO: 269) |
| | Chothia (SEQ ID NO: 34) | Chothia (SEQ ID NO: 288) |
| | IMGT (SEQ ID NO: 59) | IMGT (SEQ ID NO: 313) |
| | CDR2: Kabat (SEQ ID NO: 88) | CDR2: Kabat (SEQ ID NO: 349) |
| | Chothia (SEQ ID NO: 125) | Chothia (SEQ ID NO: 365) |
| | IMGT (SEQ ID NO: 156) | IMGT (SEQ ID NO: 365) |
| | CDR3: Kabat (SEQ ID NO: 190) | CDR3: Kabat (SEQ ID NO: 382) |
| | Chothia (SEQ ID NO: 215) | Chothia (SEQ ID NO: 407) |
| | IMGT (SEQ ID NO: 240) | IMGT (SEQ ID NO: 382) |
| 536 | VH (SEQ ID NO: 456) | VL (SEQ ID NO: 517) |
| | CDR1: Kabat (SEQ ID NO: 4) | CDR1: Kabat (SEQ ID NO: 260) |
| | Chothia (SEQ ID NO: 22) | Chothia (SEQ ID NO: 288) |
| | IMGT (SEQ ID NO: 47) | IMGT (SEQ ID NO: 313) |
| | CDR2: Kabat (SEQ ID NO: 89) | CDR2: Kabat (SEQ ID NO: 337) |
| | Chothia (SEQ ID NO: 126) | Chothia (SEQ ID NO: 361) |
| | IMGT (SEQ ID NO: 157) | IMGT (SEQ ID NO: 361) |
| | CDR3: Kabat (SEQ ID NO: 178) | CDR3: Kabat (SEQ ID NO: 371) |
| | Chothia (SEQ ID NO: 206) | Chothia (SEQ ID NO: 397) |
| | IMGT (SEQ ID NO: 226) | IMGT (SEQ ID NO: 371) |
| 537 | VH (SEQ ID NO: 457) | VL (SEQ ID NO: 518) |
| | CDR1: Kabat (SEQ ID NO: 4) | CDR1: Kabat (SEQ ID NO: 271) |
| | Chothia (SEQ ID NO: 35) | Chothia (SEQ ID NO: 296) |
| | IMGT (SEQ ID NO: 60) | IMGT (SEQ ID NO: 320) |
| | CDR2: Kabat (SEQ ID NO: 72) | CDR2: Kabat (SEQ ID NO: 350) |
| | Chothia (SEQ ID NO: 113) | Chothia (SEQ ID NO: 361) |
| | IMGT (SEQ ID NO: 144) | IMGT (SEQ ID NO: 361) |
| | CDR3: Kabat (SEQ ID NO: 178) | CDR3: Kabat (SEQ ID NO: 371) |
| | Chothia (SEQ ID NO: 206) | Chothia (SEQ ID NO: 397) |
| | IMGT (SEQ ID NO: 241) | IMGT (SEQ ID NO: 371) |
| 538 | VH (SEQ ID NO: 458) | VL (SEQ ID NO: 492) |
| | CDR1: Kabat (SEQ ID NO: 4) | CDR1: Kabat (SEQ ID NO: 261) |
| | Chothia (SEQ ID NO: 22) | Chothia (SEQ ID NO: 289) |
| | IMGT (SEQ ID NO: 47) | IMGT (SEQ ID NO: 314) |
| | CDR2: Kabat (SEQ ID NO: 90) | CDR2: Kabat (SEQ ID NO: 337) |
| | Chothia (SEQ ID NO: 127) | Chothia (SEQ ID NO: 361) |
| | IMGT (SEQ ID NO: 158) | IMGT (SEQ ID NO: 361) |
| | CDR3: Kabat (SEQ ID NO: 178) | CDR3: Kabat (SEQ ID NO: 371) |
| | Chothia (SEQ ID NO: 206) | Chothia (SEQ ID NO: 397) |
| | IMGT (SEQ ID NO: 226) | IMGT (SEQ ID NO: 371) |
| 539 | VH (SEQ ID NO: 459) | VL (SEQ ID NO: 519) |
| | CDR1: Kabat (SEQ ID NO: 16) | CDR1: Kabat (SEQ ID NO: 269) |
| | Chothia (SEQ ID NO: 36) | Chothia (SEQ ID NO: 288) |
| | IMGT (SEQ ID NO: 61) | IMGT (SEQ ID NO: 313) |
| | CDR2: Kabat (SEQ ID NO: 91) | CDR2: Kabat (SEQ ID NO: 351) |
| | Chothia (SEQ ID NO: 128) | Chothia (SEQ ID NO: 361) |
| | IMGT (SEQ ID NO: 159) | IMGT (SEQ ID NO: 361) |
| | CDR3: Kabat (SEQ ID NO: 191) | CDR3: Kabat (SEQ ID NO: 382) |
| | Chothia (SEQ ID NO: 215) | Chothia (SEQ ID NO: 407) |
| | IMGT (SEQ ID NO: 242) | IMGT (SEQ ID NO: 382) |
| 540 | VH (SEQ ID NO: 460) | VL (SEQ ID NO: 520) |
| | CDR1: Kabat (SEQ ID NO: 16) | CDR1: Kabat (SEQ ID NO: 269) |
| | Chothia (SEQ ID NO: 36) | Chothia (SEQ ID NO: 288) |
| | IMGT (SEQ ID NO: 61) | IMGT (SEQ ID NO: 313) |
| | CDR2: Kabat (SEQ ID NO: 91) | CDR2: Kabat (SEQ ID NO: 351) |
| | Chothia (SEQ ID NO: 128) | Chothia (SEQ ID NO: 361) |
| | IMGT (SEQ ID NO: 159) | IMGT (SEQ ID NO: 361) |
| | CDR3: Kabat (SEQ ID NO: 190) | CDR3: Kabat (SEQ ID NO: 371) |
| | Chothia (SEQ ID NO: 215) | Chothia (SEQ ID NO: 397) |
| | IMGT (SEQ ID NO: 240) | IMGT (SEQ ID NO: 371) |
| 541 | VH (SEQ ID NO: 461) | VL (SEQ ID NO: 521) |
| | CDR1: Kabat (SEQ ID NO: 15) | CDR1: Kabat (SEQ ID NO: 269) |
| | Chothia (SEQ ID NO: 27) | Chothia (SEQ ID NO: 288) |
| | IMGT (SEQ ID NO: 62) | IMGT (SEQ ID NO: 313) |
| | CDR2: Kabat (SEQ ID NO: 72) | CDR2: Kabat (SEQ ID NO: 337) |
| | Chothia (SEQ ID NO: 113) | Chothia (SEQ ID NO: 361) |
| | IMGT (SEQ ID NO: 144) | IMGT (SEQ ID NO: 361) |
| | CDR3: Kabat (SEQ ID NO: 190) | CDR3: Kabat (SEQ ID NO: 382) |
| | Chothia (SEQ ID NO: 215) | Chothia (SEQ ID NO: 407) |
| | IMGT (SEQ ID NO: 240) | IMGT (SEQ ID NO: 382) |
| 542 | VH (SEQ ID NO: 461) | VL (SEQ ID NO: 522) |
| | CDR1: Kabat (SEQ ID NO: 15) | CDR1: Kabat (SEQ ID NO: 269) |
| | Chothia (SEQ ID NO: 27) | Chothia (SEQ ID NO: 288) |
| | IMGT (SEQ ID NO: 62) | IMGT (SEQ ID NO: 313) |
| | CDR2: Kabat (SEQ ID NO: 72) | CDR2: Kabat (SEQ ID NO: 351) |
| | Chothia (SEQ ID NO: 113) | Chothia (SEQ ID NO: 361) |
| | IMGT (SEQ ID NO: 144) | IMGT (SEQ ID NO: 361) |
| | CDR3: Kabat (SEQ ID NO: 190) | CDR3: Kabat (SEQ ID NO: 383) |
| | Chothia (SEQ ID NO: 215) | Chothia (SEQ ID NO: 408) |
| | IMGT (SEQ ID NO: 240) | IMGT (SEQ ID NO: 383) |
| 543 | VH (SEQ ID NO: 462) | VL (SEQ ID NO: 523) |
| | CDR1: Kabat (SEQ ID NO: 5) | CDR1: Kabat (SEQ ID NO: 272) |
| | Chothia (SEQ ID NO: 37) | Chothia (SEQ ID NO: 297) |
| | IMGT (SEQ ID NO: 63) | IMGT (SEQ ID NO: 321) |
| | CDR2: Kabat (SEQ ID NO: 92) | CDR2: Kabat (SEQ ID NO: 338) |
| | Chothia (SEQ ID NO: 129) | Chothia (SEQ ID NO: 362) |
| | IMGT (SEQ ID NO: 160) | IMGT (SEQ ID NO: 362) |
| | CDR3: Kabat (SEQ ID NO: 179) | CDR3: Kabat (SEQ ID NO: 384) |
| | Chothia (SEQ ID NO: 207) | Chothia (SEQ ID NO: 409) |
| | IMGT (SEQ ID NO: 227) | IMGT (SEQ ID NO: 384) |
| 544 | VH (SEQ ID NO: 463) | VL (SEQ ID NO: 523) |
| | CDR1: Kabat (SEQ ID NO: 5) | CDR1: Kabat (SEQ ID NO: 272) |
| | Chothia (SEQ ID NO: 24) | Chothia (SEQ ID NO: 297) |
| | IMGT (SEQ ID NO: 49) | IMGT (SEQ ID NO: 321) |
| | CDR2: Kabat (SEQ ID NO: 93) | CDR2: Kabat (SEQ ID NO: 338) |
| | Chothia (SEQ ID NO: 130) | Chothia (SEQ ID NO: 362) |
| | IMGT (SEQ ID NO: 161) | IMGT (SEQ ID NO: 362) |
| | CDR3: Kabat (SEQ ID NO: 179) | CDR3: Kabat (SEQ ID NO: 384) |
| | Chothia (SEQ ID NO: 207) | Chothia (SEQ ID NO: 409) |
| | IMGT (SEQ ID NO: 227) | IMGT (SEQ ID NO: 384) |
| 545 | VH (SEQ ID NO: 464) | VL (SEQ ID NO: 524) |
| | CDR1: Kabat (SEQ ID NO: 5) | CDR1: Kabat (SEQ ID NO: 273) |
| | Chothia (SEQ ID NO: 840) | Chothia (SEQ ID NO: 298) |
| | IMGT (SEQ ID NO: 48) | IMGT (SEQ ID NO: 322) |
| | CDR2: Kabat (SEQ ID NO: 94) | CDR2: Kabat (SEQ ID NO: 338) |
| | Chothia (SEQ ID NO: 131) | Chothia (SEQ ID NO: 362) |
| | IMGT (SEQ ID NO: 162) | IMGT (SEQ ID NO: 362) |
| | CDR3: Kabat (SEQ ID NO: 192) | CDR3: Kabat (SEQ ID NO: 385) |
| | Chothia (SEQ ID NO: 207) | Chothia (SEQ ID NO: 410) |
| | IMGT (SEQ ID NO: 243) | IMGT (SEQ ID NO: 385) |
| 546 | VH (SEQ ID NO: 465) | VL (SEQ ID NO: 525) |
| | CDR1: Kabat (SEQ ID NO: 17) | CDR1: Kabat (SEQ ID NO: 272) |
| | Chothia (SEQ ID NO: 38) | Chothia (SEQ ID NO: 297) |
| | IMGT (SEQ ID NO: 64) | IMGT (SEQ ID NO: 321) |
| | CDR2: Kabat (SEQ ID NO: 95) | CDR2: Kabat (SEQ ID NO: 338) |
| | Chothia (SEQ ID NO: 132) | Chothia (SEQ ID NO: 362) |
| | IMGT (SEQ ID NO: 163) | IMGT (SEQ ID NO: 362) |
| | CDR3: Kabat (SEQ ID NO: 179) | CDR3: Kabat (SEQ ID NO: 386) |
| | Chothia (SEQ ID NO: 207) | Chothia (SEQ ID NO: 411) |
| | IMGT (SEQ ID NO: 227) | IMGT (SEQ ID NO: 386) |
| 547 | VH (SEQ ID NO: 466) | VL (SEQ ID NO: 526) |
| | CDR1: Kabat (SEQ ID NO: 5) | CDR1: Kabat (SEQ ID NO: 274) |
| | Chothia (SEQ ID NO: 24) | Chothia (SEQ ID NO: 299) |
| | IMGT (SEQ ID NO: 49) | IMGT (SEQ ID NO: 323) |
| | CDR2: Kabat (SEQ ID NO: 96) | CDR2: Kabat (SEQ ID NO: 338) |
| | Chothia (SEQ ID NO: 130) | Chothia (SEQ ID NO: 362) |
| | IMGT (SEQ ID NO: 164) | IMGT (SEQ ID NO: 362) |
| | CDR3: Kabat (SEQ ID NO: 192) | CDR3: Kabat (SEQ ID NO: 386) |
| | Chothia (SEQ ID NO: 207) | Chothia (SEQ ID NO: 411) |
| | IMGT (SEQ ID NO: 243) | IMGT (SEQ ID NO: 386) |
| 548 | VH (SEQ ID NO: 467) | VL (SEQ ID NO: 527) |
| | CDR1: Kabat (SEQ ID NO: 5) | CDR1: Kabat (SEQ ID NO: 275) |
| | Chothia (SEQ ID NO: 24) | Chothia (SEQ ID NO: 300) |
| | IMGT (SEQ ID NO: 49) | IMGT (SEQ ID NO: 324) |
| | CDR2: Kabat (SEQ ID NO: 97) | CDR2: Kabat (SEQ ID NO: 338) |
| | Chothia (SEQ ID NO: 130) | Chothia (SEQ ID NO: 362) |
| | IMGT (SEQ ID NO: 164) | IMGT (SEQ ID NO: 362) |
| | CDR3: Kabat (SEQ ID NO: 179) | CDR3: Kabat (SEQ ID NO: 386) |
| | Chothia (SEQ ID NO: 207) | Chothia (SEQ ID NO: 411) |
| | IMGT (SEQ ID NO: 227) | IMGT (SEQ ID NO: 386) |
| 549 | VH (SEQ ID NO: 468) | VL (SEQ ID NO: 528) |
| | CDR1: Kabat (SEQ ID NO: 18) | CDR1: Kabat (SEQ ID NO: 276) |
| | Chothia (SEQ ID NO: 39) | Chothia (SEQ ID NO: 301) |
| | IMGT (SEQ ID NO: 65) | IMGT (SEQ ID NO: 325) |
| | CDR2: Kabat (SEQ ID NO: 98) | CDR2: Kabat (SEQ ID NO: 337) |
| | Chothia (SEQ ID NO: 116) | Chothia (SEQ ID NO: 361) |
| | IMGT (SEQ ID NO: 147) | IMGT (SEQ ID NO: 361) |
| | CDR3: Kabat (SEQ ID NO: 193) | CDR3: Kabat (SEQ ID NO: 387) |
| | Chothia (SEQ ID NO: 216) | Chothia (SEQ ID NO: 412) |
| | IMGT (SEQ ID NO: 244) | IMGT (SEQ ID NO: 387) |
| 550 | VH (SEQ ID NO: 469) | VL (SEQ ID NO: 529) |
| | CDR1: Kabat (SEQ ID NO: 18) | CDR1: Kabat (SEQ ID NO: 277) |
| | Chothia (SEQ ID NO: 39) | Chothia (SEQ ID NO: 302) |
| | IMGT (SEQ ID NO: 65) | IMGT (SEQ ID NO: 326) |
| | CDR2: Kabat (SEQ ID NO: 98) | CDR2: Kabat (SEQ ID NO: 337) |
| | Chothia (SEQ ID NO: 116) | Chothia (SEQ ID NO: 361) |
| | IMGT (SEQ ID NO: 147) | IMGT (SEQ ID NO: 361) |
| | CDR3: Kabat (SEQ ID NO: 193) | CDR3: Kabat (SEQ ID NO: 387) |
| | Chothia (SEQ ID NO: 216) | Chothia (SEQ ID NO: 412) |
| | IMGT (SEQ ID NO: 245) | IMGT (SEQ ID NO: 387) |
| 551 | VH (SEQ ID NO: 470) | VL (SEQ ID NO: 528) |
| | CDR1: Kabat (SEQ ID NO: 18) | CDR1: Kabat (SEQ ID NO: 276) |
| | Chothia (SEQ ID NO: 39) | Chothia (SEQ ID NO: 301) |
| | IMGT (SEQ ID NO: 65) | IMGT (SEQ ID NO: 325) |
| | CDR2: Kabat (SEQ ID NO: 98) | CDR2: Kabat (SEQ ID NO: 337) |
| | Chothia (SEQ ID NO: 116) | Chothia (SEQ ID NO: 361) |
| | IMGT (SEQ ID NO: 147) | IMGT (SEQ ID NO: 361) |
| | CDR3: Kabat (SEQ ID NO: 194) | CDR3: Kabat (SEQ ID NO: 387) |
| | Chothia (SEQ ID NO: 217) | Chothia (SEQ ID NO: 412) |
| | IMGT (SEQ ID NO: 246) | IMGT (SEQ ID NO: 387) |
| 552 | VH (SEQ ID NO: 471) | VL (SEQ ID NO: 530) |
| | CDR1: Kabat (SEQ ID NO: 18) | CDR1: Kabat (SEQ ID NO: 276) |
| | Chothia (SEQ ID NO: 39) | Chothia (SEQ ID NO: 301) |
| | IMGT (SEQ ID NO: 65) | IMGT (SEQ ID NO: 325) |
| | CDR2: Kabat (SEQ ID NO: 98) | CDR2: Kabat (SEQ ID NO: 337) |
| | Chothia (SEQ ID NO: 116) | Chothia (SEQ ID NO: 361) |
| | IMGT (SEQ ID NO: 147) | IMGT (SEQ ID NO: 361) |
| | CDR3: Kabat (SEQ ID NO: 194) | CDR3: Kabat (SEQ ID NO: 388) |
| | Chothia (SEQ ID NO: 217) | Chothia (SEQ ID NO: 413) |
| | IMGT (SEQ ID NO: 247) | IMGT (SEQ ID NO: 388) |
| 553 | VH (SEQ ID NO: 472) | VL (SEQ ID NO: 531) |
| | CDR1: Kabat (SEQ ID NO: 19) | CDR1: Kabat (SEQ ID NO: 278) |
| | Chothia (SEQ ID NO: 40) | Chothia (SEQ ID NO: 303) |
| | IMGT (SEQ ID NO: 66) | IMGT (SEQ ID NO: 327) |
| | CDR2: Kabat (SEQ ID NO: 99) | CDR2: Kabat (SEQ ID NO: 352) |
| | Chothia (SEQ ID NO: 133) | Chothia (SEQ ID NO: 367) |
| | IMGT (SEQ ID NO: 165) | IMGT (SEQ ID NO: 367) |
| | CDR3: Kabat (SEQ ID NO: 180) | CDR3: Kabat (SEQ ID NO: 387) |
| | Chothia (SEQ ID NO: 208) | Chothia (SEQ ID NO: 412) |
| | IMGT (SEQ ID NO: 248) | IMGT (SEQ ID NO: 387) |
| 554 | VH (SEQ ID NO: 473) | VL (SEQ ID NO: 532) |
| | CDR1: Kabat (SEQ ID NO: 20) | CDR1: Kabat (SEQ ID NO: 279) |
| | Chothia (SEQ ID NO: 41) | Chothia (SEQ ID NO: 304) |
| | IMGT (SEQ ID NO: 67) | IMGT (SEQ ID NO: 328) |
| | CDR2: Kabat (SEQ ID NO: 100) | CDR2: Kabat (SEQ ID NO: 353) |
| | Chothia (SEQ ID NO: 134) | Chothia (SEQ ID NO: 361) |
| | IMGT (SEQ ID NO: 166) | IMGT (SEQ ID NO: 361) |
| | CDR3: Kabat (SEQ ID NO: 195) | CDR3: Kabat (SEQ ID NO: 374) |
| | Chothia (SEQ ID NO: 218) | Chothia (SEQ ID NO: 400) |
| | IMGT (SEQ ID NO: 249) | IMGT (SEQ ID NO: 374) |
| 555 | VH (SEQ ID NO: 474) | VL (SEQ ID NO: 533) |
| | CDR1: Kabat (SEQ ID NO: 20) | CDR1: Kabat (SEQ ID NO: 280) |
| | Chothia (SEQ ID NO: 840) | Chothia (SEQ ID NO: 305) |
| | IMGT (SEQ ID NO: 48) | IMGT (SEQ ID NO: 329) |
| | CDR2: Kabat (SEQ ID NO: 101) | CDR2: Kabat (SEQ ID NO: 337) |
| | Chothia (SEQ ID NO: 133) | Chothia (SEQ ID NO: 361) |
| | IMGT (SEQ ID NO: 167) | IMGT (SEQ ID NO: 361) |
| | CDR3: Kabat (SEQ ID NO: 180) | CDR3: Kabat (SEQ ID NO: 389) |
| | Chothia (SEQ ID NO: 208) | Chothia (SEQ ID NO: 414) |
| | IMGT (SEQ ID NO: 248) | IMGT (SEQ ID NO: 389) |
| 556 | VH (SEQ ID NO: 475) | VL (SEQ ID NO: 534) |
| | CDR1: Kabat (SEQ ID NO: 5) | CDR1: Kabat (SEQ ID NO: 280) |
| | Chothia (SEQ ID NO: 840) | Chothia (SEQ ID NO: 305) |
| | IMGT (SEQ ID NO: 48) | IMGT (SEQ ID NO: 329) |
| | CDR2: Kabat (SEQ ID NO: 102) | CDR2: Kabat (SEQ ID NO: 350) |
| | Chothia (SEQ ID NO: 135) | Chothia (SEQ ID NO: 361) |
| | IMGT (SEQ ID NO: 168) | IMGT (SEQ ID NO: 361) |
| | CDR3: Kabat (SEQ ID NO: 180) | CDR3: Kabat (SEQ ID NO: 389) |
| | Chothia (SEQ ID NO: 208) | Chothia (SEQ ID NO: 414) |
| | IMGT (SEQ ID NO: 248) | IMGT (SEQ ID NO: 389) |
| 557 | VH (SEQ ID NO: 476) | VL (SEQ ID NO: 535) |
| | CDR1: Kabat (SEQ ID NO: 5) | CDR1: Kabat (SEQ ID NO: 280) |
| | Chothia (SEQ ID NO: 840) | Chothia (SEQ ID NO: 305) |
| | IMGT (SEQ ID NO: 48) | IMGT (SEQ ID NO: 329) |
| | CDR2: Kabat (SEQ ID NO: 103) | CDR2: Kabat (SEQ ID NO: 354) |
| | Chothia (SEQ ID NO: 136) | Chothia (SEQ ID NO: 367) |
| | IMGT (SEQ ID NO: 169) | IMGT (SEQ ID NO: 367) |
| | CDR3: Kabat (SEQ ID NO: 196) | CDR3: Kabat (SEQ ID NO: 390) |
| | Chothia (SEQ ID NO: 218) | Chothia (SEQ ID NO: 415) |
| | IMGT (SEQ ID NO: 250) | IMGT (SEQ ID NO: 390) |
| 558 | VH (SEQ ID NO: 477) | VL (SEQ ID NO: 536) |
| | CDR1: Kabat (SEQ ID NO: 5) | CDR1: Kabat (SEQ ID NO: 281) |
| | Chothia (SEQ ID NO: 840) | Chothia (SEQ ID NO: 306) |
| | IMGT (SEQ ID NO: 48) | IMGT (SEQ ID NO: 330) |
| | CDR2: Kabat (SEQ ID NO: 104) | CDR2: Kabat (SEQ ID NO: 337) |
| | Chothia (SEQ ID NO: 137) | Chothia (SEQ ID NO: 361) |
| | IMGT (SEQ ID NO: 170) | IMGT (SEQ ID NO: 361) |
| | CDR3: Kabat (SEQ ID NO: 197) | CDR3: Kabat (SEQ ID NO: 387) |
| | Chothia (SEQ ID NO: 208) | Chothia (SEQ ID NO: 412) |
| | IMGT (SEQ ID NO: 251) | IMGT (SEQ ID NO: 387) |
| 559 | VH (SEQ ID NO: 478) | VL (SEQ ID NO: 536) |
| | CDR1: Kabat (SEQ ID NO: 5) | CDR1: Kabat (SEQ ID NO: 281) |
| | Chothia (SEQ ID NO: 840) | Chothia (SEQ ID NO: 306) |
| | IMGT (SEQ ID NO: 48) | IMGT (SEQ ID NO: 330) |
| | CDR2: Kabat (SEQ ID NO: 105) | CDR2: Kabat (SEQ ID NO: 337) |
| | Chothia (SEQ ID NO: 138) | Chothia (SEQ ID NO: 361) |
| | IMGT (SEQ ID NO: 171) | IMGT (SEQ ID NO: 361) |
| | CDR3: Kabat (SEQ ID NO: 197) | CDR3: Kabat (SEQ ID NO: 387) |
| | Chothia (SEQ ID NO: 208) | Chothia (SEQ ID NO: 412) |
| | IMGT (SEQ ID NO: 251) | IMGT (SEQ ID NO: 387) |
| 560 | VH (SEQ ID NO: 479) | VL (SEQ ID NO: 537) |
| | CDR1: Kabat (SEQ ID NO: 5) | CDR1: Kabat (SEQ ID NO: 282) |
| | Chothia (SEQ ID NO: 840) | Chothia (SEQ ID NO: 307) |
| | IMGT (SEQ ID NO: 48) | IMGT (SEQ ID NO: 331) |
| | CDR2: Kabat (SEQ ID NO: 94) | CDR2: Kabat (SEQ ID NO: 337) |
| | Chothia (SEQ ID NO: 131) | Chothia (SEQ ID NO: 361) |
| | IMGT (SEQ ID NO: 162) | IMGT (SEQ ID NO: 361) |
| | CDR3: Kabat (SEQ ID NO: 198) | CDR3: Kabat (SEQ ID NO: 391) |
| | Chothia (SEQ ID NO: 219) | Chothia (SEQ ID NO: 416) |
| | IMGT (SEQ ID NO: 252) | IMGT (SEQ ID NO: 391) |
| 561 | VH (SEQ ID NO: 480) | VL (SEQ ID NO: 538) |
| | CDR1: Kabat (SEQ ID NO: 20) | CDR1: Kabat (SEQ ID NO: 280) |
| | Chothia (SEQ ID NO: 840) | Chothia (SEQ ID NO: 305) |
| | IMGT (SEQ ID NO: 48) | IMGT (SEQ ID NO: 329) |
| | CDR2: Kabat (SEQ ID NO: 94) | CDR2: Kabat (SEQ ID NO: 355) |
| | Chothia (SEQ ID NO: 131) | Chothia (SEQ ID NO: 367) |
| | IMGT (SEQ ID NO: 162) | IMGT (SEQ ID NO: 367) |
| | CDR3: Kabat (SEQ ID NO: 195) | CDR3: Kabat (SEQ ID NO: 387) |
| | Chothia (SEQ ID NO: 218) | Chothia (SEQ ID NO: 412) |
| | IMGT (SEQ ID NO: 253) | IMGT (SEQ ID NO: 387) |
| 562 | VH (SEQ ID NO: 481) | VL (SEQ ID NO: 539) |
| | CDR1: Kabat (SEQ ID NO: 5) | CDR1: Kabat (SEQ ID NO: 280) |
| | Chothia (SEQ ID NO: 840) | Chothia (SEQ ID NO: 305) |
| | IMGT (SEQ ID NO: 48) | IMGT (SEQ ID NO: 329) |
| | CDR2: Kabat (SEQ ID NO: 94) | CDR2: Kabat (SEQ ID NO: 337) |
| | Chothia (SEQ ID NO: 131) | Chothia (SEQ ID NO: 361) |
| | IMGT (SEQ ID NO: 162) | IMGT (SEQ ID NO: 361) |
| | CDR3: Kabat (SEQ ID NO: 180) | CDR3: Kabat (SEQ ID NO: 387) |
| | Chothia (SEQ ID NO: 208) | Chothia (SEQ ID NO: 412) |
| | IMGT (SEQ ID NO: 248) | IMGT (SEQ ID NO: 387) |
| 563 | VH (SEQ ID NO: 482) | VL (SEQ ID NO: 539) |
| | CDR1: Kabat (SEQ ID NO: 6) | CDR1: Kabat (SEQ ID NO: 280) |
| | Chothia (SEQ ID NO: 840) | Chothia (SEQ ID NO: 305) |
| | IMGT (SEQ ID NO: 48) | IMGT (SEQ ID NO: 329) |
| | CDR2: Kabat (SEQ ID NO: 106) | CDR2: Kabat (SEQ ID NO: 337) |
| | Chothia (SEQ ID NO: 137) | Chothia (SEQ ID NO: 361) |
| | IMGT (SEQ ID NO: 170) | IMGT (SEQ ID NO: 361) |
| | CDR3: Kabat (SEQ ID NO: 195) | CDR3: Kabat (SEQ ID NO: 387) |
| | Chothia (SEQ ID NO: 218) | Chothia (SEQ ID NO: 412) |
| | IMGT (SEQ ID NO: 253) | IMGT (SEQ ID NO: 387) |
| 564 | VH (SEQ ID NO: 483) | VL (SEQ ID NO: 539) |
| | CDR1: Kabat (SEQ ID NO: 20) | CDR1: Kabat (SEQ ID NO: 280) |
| | Chothia (SEQ ID NO: 41) | Chothia (SEQ ID NO: 305) |
| | IMGT (SEQ ID NO: 67) | IMGT (SEQ ID NO: 329) |
| | CDR2: Kabat (SEQ ID NO: 107) | CDR2: Kabat (SEQ ID NO: 337) |
| | Chothia (SEQ ID NO: 133) | Chothia (SEQ ID NO: 361) |
| | IMGT (SEQ ID NO: 167) | IMGT (SEQ ID NO: 361) |
| | CDR3: Kabat (SEQ ID NO: 180) | CDR3: Kabat (SEQ ID NO: 387) |
| | Chothia (SEQ ID NO: 208) | Chothia (SEQ ID NO: 412) |
| | IMGT (SEQ ID NO: 248) | IMGT (SEQ ID NO: 387) |
| 565 | VH (SEQ ID NO: 484) | VL (SEQ ID NO: 540) |
| | CDR1: Kabat (SEQ ID NO: 5) | CDR1: Kabat (SEQ ID NO: 283) |
| | Chothia (SEQ ID NO: 840) | Chothia (SEQ ID NO: 308) |
| | IMGT (SEQ ID NO: 48) | IMGT (SEQ ID NO: 332) |
| | CDR2: Kabat (SEQ ID NO: 108) | CDR2: Kabat (SEQ ID NO: 356) |
| | Chothia (SEQ ID NO: 139) | Chothia (SEQ ID NO: 361) |
| | IMGT (SEQ ID NO: 172) | IMGT (SEQ ID NO: 361) |
| | CDR3: Kabat (SEQ ID NO: 181) | CDR3: Kabat (SEQ ID NO: 374) |
| | Chothia (SEQ ID NO: 209) | Chothia (SEQ ID NO: 400) |
| | IMGT (SEQ ID NO: 229) | IMGT (SEQ ID NO: 374) |
| 566 | VH (SEQ ID NO: 485) | VL (SEQ ID NO: 541) |
| | CDR1: Kabat (SEQ ID NO: 21) | CDR1: Kabat (SEQ ID NO: 280) |
| | Chothia (SEQ ID NO: 42) | Chothia (SEQ ID NO: 305) |
| | IMGT (SEQ ID NO: 68) | IMGT (SEQ ID NO: 329) |
| | CDR2: Kabat (SEQ ID NO: 109) | CDR2: Kabat (SEQ ID NO: 337) |
| | Chothia (SEQ ID NO: 140) | Chothia (SEQ ID NO: 361) |
| | IMGT (SEQ ID NO: 173) | IMGT (SEQ ID NO: 361) |
| | CDR3: Kabat (SEQ ID NO: 199) | CDR3: Kabat (SEQ ID NO: 374) |
| | Chothia (SEQ ID NO: 220) | Chothia (SEQ ID NO: 400) |
| | IMGT (SEQ ID NO: 254) | IMGT (SEQ ID NO: 374) |
| 567 | VH (SEQ ID NO: 486) | VL (SEQ ID NO: 542) |
| | CDR1: Kabat (SEQ ID NO: 5) | CDR1: Kabat (SEQ ID NO: 284) |
| | Chothia (SEQ ID NO: 43) | Chothia (SEQ ID NO: 309) |
| | IMGT (SEQ ID NO: 69) | IMGT (SEQ ID NO: 333) |
| | CDR2: Kabat (SEQ ID NO: 106) | CDR2: Kabat (SEQ ID NO: 337) |
| | Chothia (SEQ ID NO: 137) | Chothia (SEQ ID NO: 361) |
| | IMGT (SEQ ID NO: 170) | IMGT (SEQ ID NO: 361) |
| | CDR3: Kabat (SEQ ID NO: 200) | CDR3: Kabat (SEQ ID NO: 374) |
| | Chothia (SEQ ID NO: 221) | Chothia (SEQ ID NO: 400) |
| | IMGT (SEQ ID NO: 255) | IMGT (SEQ ID NO: 374) |
| 568 | VH (SEQ ID NO: 487) | VL (SEQ ID NO: 543) |
| | CDR1: Kabat (SEQ ID NO: 5) | CDR1: Kabat (SEQ ID NO: 285) |
| | Chothia (SEQ ID NO: 43) | Chothia (SEQ ID NO: 310) |
| | IMGT (SEQ ID NO: 69) | IMGT (SEQ ID NO: 334) |
| | CDR2: Kabat (SEQ ID NO: 94) | CDR2: Kabat (SEQ ID NO: 357) |
| | Chothia (SEQ ID NO: 131) | Chothia (SEQ ID NO: 361) |
| | IMGT (SEQ ID NO: 162) | IMGT (SEQ ID NO: 361) |
| | CDR3: Kabat (SEQ ID NO: 201) | CDR3: Kabat (SEQ ID NO: 392) |
| | Chothia (SEQ ID NO: 222) | Chothia (SEQ ID NO: 417) |
| | IMGT (SEQ ID NO: 256) | IMGT (SEQ ID NO: 392) |
| 569 | VH (SEQ ID NO: 488) | VL (SEQ ID NO: 544) |
| | CDR1: Kabat (SEQ ID NO: 5) | CDR1: Kabat (SEQ ID NO: 285) |
| | Chothia (SEQ ID NO: 43) | Chothia (SEQ ID NO: 310) |
| | IMGT (SEQ ID NO: 69) | IMGT (SEQ ID NO: 334) |
| | CDR2: Kabat (SEQ ID NO: 110) | CDR2: Kabat (SEQ ID NO: 358) |
| | Chothia (SEQ ID NO: 141) | Chothia (SEQ ID NO: 361) |
| | IMGT (SEQ ID NO: 174) | IMGT (SEQ ID NO: 361) |
| | CDR3: Kabat (SEQ ID NO: 202) | CDR3: Kabat (SEQ ID NO: 393) |
| | Chothia (SEQ ID NO: 222) | Chothia (SEQ ID NO: 418) |
| | IMGT (SEQ ID NO: 257) | IMGT (SEQ ID NO: 393) |
| 104 | VH (SEQ ID NO: 818) | VL (SEQ ID NO: 825) |
| | CDR1: Kabat (SEQ ID NO: 5) | CDR1: Kabat (SEQ ID NO: 781) |
| | Chothia (SEQ ID NO: 840) | Chothia (SEQ ID NO: 787) |
| | IMGT (SEQ ID NO: 48) | IMGT (SEQ ID NO: 792) |
| | CDR2: Kabat (SEQ ID NO: 748) | CDR2: Kabat (SEQ ID NO: 797) |
| | Chothia (SEQ ID NO: 116) | Chothia (SEQ ID NO: 802) |
| | IMGT (SEQ ID NO: 758) | IMGT (SEQ ID NO: 802) |
| | CDR3: Kabat (SEQ ID NO: 763) | CDR3: Kabat (SEQ ID NO: 806) |
| | Chothia (SEQ ID NO: 769) | Chothia (SEQ ID NO: 812) |
| | IMGT (SEQ ID NO: 775) | IMGT (SEQ ID NO: 806) |
| 111 | VH (SEQ ID NO: 819) | VL (SEQ ID NO: 826) |
| | CDR1: Kabat (SEQ ID NO: 740) | CDR1: Kabat (SEQ ID NO: 782) |
| | Chothia (SEQ ID NO: 742) | Chothia (SEQ ID NO: 788) |
| | IMGT (SEQ ID NO: 745) | IMGT (SEQ ID NO: 793) |
| | CDR2: Kabat (SEQ ID NO: 749) | CDR2: Kabat (SEQ ID NO: 798) |
| | Chothia (SEQ ID NO: 754) | Chothia (SEQ ID NO: 803) |
| | IMGT (SEQ ID NO: 759) | IMGT (SEQ ID NO: 803) |
| | CDR3: Kabat (SEQ ID NO: 764) | CDR3: Kabat (SEQ ID NO: 807) |
| | Chothia (SEQ ID NO: 770) | Chothia (SEQ ID NO: 813) |
| | IMGT (SEQ ID NO: 776) | IMGT (SEQ ID NO: 807) |
| 115 | VH (SEQ ID NO: 820) | VL (SEQ ID NO: 827) |
| | CDR1: Kabat (SEQ ID NO: 17) | CDR1: Kabat (SEQ ID NO: 783) |
| | Chothia (SEQ ID NO: 743) | Chothia (SEQ ID NO: 298) |
| | IMGT (SEQ ID NO: 746) | IMGT (SEQ ID NO: 322) |
| | CDR2: Kabat (SEQ ID NO: 750) | CDR2: Kabat (SEQ ID NO: 799) |
| | Chothia (SEQ ID NO: 133) | Chothia (SEQ ID NO: 804) |
| | IMGT (SEQ ID NO: 165) | IMGT (SEQ ID NO: 804) |
| | CDR3: Kabat (SEQ ID NO: 765) | CDR3: Kabat (SEQ ID NO: 808) |
| | Chothia (SEQ ID NO: 771) | Chothia (SEQ ID NO: 814) |
| | IMGT (SEQ ID NO: 777) | IMGT (SEQ ID NO: 808) |
| 119 | VH (SEQ ID NO: 821) | VL (SEQ ID NO: 828) |
| | CDR1: Kabat (SEQ ID NO: 5) | CDR1: Kabat (SEQ ID NO: 784) |
| | Chothia (SEQ ID NO: 840) | Chothia (SEQ ID NO: 789) |
| | IMGT (SEQ ID NO: 48) | IMGT (SEQ ID NO: 794) |
| | CDR2: Kabat (SEQ ID NO: 751) | CDR2: Kabat (SEQ ID NO: 341) |
| | Chothia (SEQ ID NO: 755) | Chothia (SEQ ID NO: 361) |
| | IMGT (SEQ ID NO: 760) | IMGT (SEQ ID NO: 361) |
| | CDR3: Kabat (SEQ ID NO: 766) | CDR3: Kabat (SEQ ID NO: 809) |
| | Chothia (SEQ ID NO: 772) | Chothia (SEQ ID NO: 815) |
| | IMGT (SEQ ID NO: 778) | IMGT (SEQ ID NO: 809) |
| 216 | VH (SEQ ID NO: 822) | VL (SEQ ID NO: 829) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 70) | CDR2: Kabat (SEQ ID NO: 337) |
| | Chothia (SEQ ID NO: 111) | Chothia (SEQ ID NO: 361) |
| | IMGT (SEQ ID NO: 142) | IMGT (SEQ ID NO: 361) |
| | CDR3: Kabat (SEQ ID NO: 175) | CDR3: Kabat (SEQ ID NO: 368) |
| | Chothia (SEQ ID NO: 203) | Chothia (SEQ ID NO: 394) |
| | IMGT (SEQ ID NO: 223) | IMGT (SEQ ID NO: 368) |
| 231 | VH (SEQ ID NO: 823) | VL (SEQ ID NO: 830) |
| | CDR1: Kabat (SEQ ID NO: 8) | CDR1: Kabat (SEQ ID NO: 785) |
| | Chothia (SEQ ID NO: 26) | Chothia (SEQ ID NO: 790) |
| | IMGT (SEQ ID NO: 51) | IMGT (SEQ ID NO: 795) |
| | CDR2: Kabat (SEQ ID NO: 752) | CDR2: Kabat (SEQ ID NO: 800) |
| | Chothia (SEQ ID NO: 756) | Chothia (SEQ ID NO: 805) |
| | IMGT (SEQ ID NO: 761) | IMGT (SEQ ID NO: 805) |
| | CDR3: Kabat (SEQ ID NO: 767) | CDR3: Kabat (SEQ ID NO: 810) |
| | Chothia (SEQ ID NO: 773) | Chothia (SEQ ID NO: 816) |
| | IMGT (SEQ ID NO: 779) | IMGT (SEQ ID NO: 810) |
| 328 | VH (SEQ ID NO: 824) | VL (SEQ ID NO: 831) |
| | CDR1: Kabat (SEQ ID NO: 741) | CDR1: Kabat (SEQ ID NO: 786) |
| | Chothia (SEQ ID NO: 744) | Chothia (SEQ ID NO: 791) |
| | IMGT (SEQ ID NO: 747) | IMGT (SEQ ID NO: 796) |
| | CDR2: Kabat (SEQ ID NO: 753) | CDR2: Kabat (SEQ ID NO: 801) |
| | Chothia (SEQ ID NO: 757) | Chothia (SEQ ID NO: 360) |
| | IMGT (SEQ ID NO: 762) | IMGT (SEQ ID NO: 360) |
| | CDR3: Kabat (SEQ ID NO: 768) | CDR3: Kabat (SEQ ID NO: 811) |
| | Chothia (SEQ ID NO: 774) | Chothia (SEQ ID NO: 817) |
| | IMGT (SEQ ID NO: 780) | IMGT (SEQ ID NO: 811) |
| 105 | VH (SEQ ID NO: 841) | VL (SEQ ID NO: 842) |
| | CDR1: Kabat (SEQ ID NO: 10) | CDR1: Kabat (SEQ ID NO: 260) |
| | Chothia (SEQ ID NO: 27) | Chothia (SEQ ID NO: 288) |
| | IMGT (SEQ ID NO: 52) | IMGT (SEQ ID NO: 313) |
| | CDR2: Kabat (SEQ ID NO: 70) | CDR2: Kabat (SEQ ID NO: 347) |
| | Chothia (SEQ ID NO: 111) | Chothia (SEQ ID NO: 365) |
| | IMGT (SEQ ID NO: 142) | IMGT (SEQ ID NO: 365) |
| | CDR3: Kabat (SEQ ID NO: 175) | CDR3: Kabat (SEQ ID NO: 378) |
| | Chothia (SEQ ID NO: 203) | Chothia (SEQ ID NO: 404) |
| | IMGT (SEQ ID NO: 234) | IMGT (SEQ ID NO: 378) |

### VH Domains

In some embodiments, an antibody, or antigen binding fragment thereof, provided herein comprises a VH sequence selected from any one of SEQ ID NOs: 419-488, 818-824, and 841.

In some embodiments, an antibody, or antigen binding fragment thereof, provided herein comprises a VH sequence having at least about 80%, 90%, 91%, 92%, 93%, 94%, 95%, 96%, 97%, 98% or 99% identity to an illustrative VH sequence selected from any one of SEQ ID NOs: 419-488, 818-824, and 841. In some embodiments, an antibody, or antigen binding fragment thereof, provided herein comprises a VH sequence selected from any one of SEQ ID NOs: 419-488, 818-824, and 841, with up to 1, 2, 3, 4, 5, 6, 7, 8, 9, 10, 11, 12, 13, 14, 15, 16, 17, 18, 19, or 20 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

### VL Domains

In some embodiments, an antibody, or antigen binding fragment thereof, provided herein comprises a VL sequence selected from any one of SEQ ID NOs: 489-544, 825-831, and 842.

In some embodiments, an antibody, or antigen binding fragment thereof, provided herein comprises a VL sequence having at least about 80%, 90%, 91%, 92%, 93%, 94%, 95%, 96%, 97%, 98% or 99% identity to a VL sequence selected from any one of SEQ ID NOs: 489-544, 825-831, and 842. In some embodiments, an antibody, or antigen binding fragment thereof, provided herein comprises a VL sequence selected from any one of SEQ ID NOs: 489-544, 825-831, and 842, with up to 1, 2, 3, 4, 5, 6, 7, 8, 9, 10, 11, 12, 13, 14, 15, 16, 17, 18, 19, or 20 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

### VH-VL Combinations

In some embodiments, an antibody, or antigen binding fragment thereof, provided herein comprises a VH sequence selected from any one of SEQ ID NOs: 419-488, 818-824, and 841; and a VL sequence selected from any one of SEQ ID NOs: 489-544, 825-831, and 842.

In certain aspects, a VH sequence selected from any one of SEQ ID NOs: 419-488, 818-824, and 841 can be combined with a VL sequence selected from any one of SEQ ID NOs: 489-544, 825-831, and 842.

In certain aspects, an antibody, or antigen binding fragment thereof, provided herein comprises a VH sequence and a VL sequence of a construct provided in TABLE 3 (e.g., a VH sequence and a VL sequence from the same row of TABLE 3) or a VH sequence and a VL sequence of a construct provided in PCT Publication No. WO2024227174, which is incorporated by reference herein in its entirety. In certain aspects, an antibody, or antigen binding fragment thereof, provided herein comprises a VH sequence and a VL sequence from Table 2 in PCT Publication No. WO2024227174 (e.g., a VH sequence and a VL sequence from the same row of Table 2).

In some embodiments, an antibody, or antigen binding fragment thereof, provided herein comprises a VH sequence having at least about 80%, 90%, 91%, 92%, 93%, 94%, 95%, 96%, 97%, 98% or 99% identity to a VH sequence selected from any one of SEQ ID NOs: 419-488, 818-824, and 841; and a VL sequence having at least about 80%, 90%, 91%, 92%, 93%, 94%, 95%, 96%, 97%, 98% or 99% identity to a VL sequence selected from any one of SEQ ID NOs: 489-544, 825-831, and 842. In some embodiments, an antibody, or antigen binding fragment thereof, provided herein comprises a VH sequence selected from any one of SEQ ID NOs: 419-488, 818-824, and 841, with up to 1, 2, 3, 4, 5, 6, 7, 8, 9, 10, 11, 12, 13, 14, 15, 16, 17, 18, 19, or 20 amino acid substitutions, and a VL sequence selected from any one of SEQ ID NOs: 489-544, 825-831, and 842, with up to 1, 2, 3, 4, 5, 6, 7, 8, 9, 10, 11, 12, 13, 14, 15, 16, 17, 18, 19, or 20 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In certain embodiments, the antibody, or antigen binding fragment thereof, comprises a VH sequence set forth in SEQ ID NO: 419 and a VL sequence set forth in SEQ ID NO: 489. In certain embodiments, the antibody, or antigen binding fragment thereof, comprises a VH sequence set forth in SEQ ID NO: 420 and a VL sequence set forth in SEQ ID NO: 490. In certain embodiments, the antibody, or antigen binding fragment thereof, comprises a VH sequence set forth in SEQ ID NO: 421 and a VL sequence set forth in SEQ ID NO: 491. In certain embodiments, the antibody, or antigen binding fragment thereof, comprises a VH sequence set forth in SEQ ID NO: 422 and a VL sequence set forth in SEQ ID NO: 492. In certain embodiments, the antibody, or antigen binding fragment thereof, comprises a VH sequence set forth in SEQ ID NO: 423 and a VL sequence set forth in SEQ ID NO: 493.

In certain embodiments, the antibody, or antigen binding fragment thereof, comprises a VH sequence set forth in SEQ ID NO: 424 and a VL sequence set forth in SEQ ID NO: 494. In certain embodiments, the antibody, or antigen binding fragment thereof, comprises a VH sequence set forth in SEQ ID NO: 425 and a VL sequence set forth in SEQ ID NO: 495. In certain embodiments, the antibody, or antigen binding fragment thereof, comprises a VH sequence set forth in SEQ ID NO: 426 and a VL sequence set forth in SEQ ID NO: 496. In certain embodiments, the antibody, or antigen binding fragment thereof, comprises a VH sequence set forth in SEQ ID NO: 427 and a VL sequence set forth in SEQ ID NO: 497. In certain embodiments, the antibody, or antigen binding fragment thereof, comprises a VH sequence set forth in SEQ ID NO: 427 and a VL sequence set forth in SEQ ID NO: 498. In certain embodiments, the antibody, or antigen binding fragment thereof, comprises a VH sequence set forth in SEQ ID NO: 428 and a VL sequence set forth in SEQ ID NO: 497. In certain embodiments, the antibody, or antigen binding fragment thereof, comprises a VH sequence set forth in SEQ ID NO: 428 and a VL sequence set forth in SEQ ID NO: 498.

In certain embodiments, the antibody, or antigen binding fragment thereof, comprises a VH sequence set forth in SEQ ID NO: 429 and a VL sequence set forth in SEQ ID NO: 497.

In certain embodiments, the antibody, or antigen binding fragment thereof, comprises a VH sequence set forth in SEQ ID NO: 429 and a VL sequence set forth in SEQ ID NO: 498.

In certain embodiments, the antibody, or antigen binding fragment thereof, comprises a VH sequence set forth in SEQ ID NO: 430 and a VL sequence set forth in SEQ ID NO: 499.

In certain embodiments, the antibody, or antigen binding fragment thereof, comprises a VH sequence set forth in SEQ ID NO: 431 and a VL sequence set forth in SEQ ID NO: 500.

In certain embodiments, the antibody, or antigen binding fragment thereof, comprises a VH sequence set forth in SEQ ID NO: 432 and a VL sequence set forth in SEQ ID NO: 501.

In certain embodiments, the antibody, or antigen binding fragment thereof, comprises a VH sequence set forth in SEQ ID NO: 433 and a VL sequence set forth in SEQ ID NO: 502.

In certain embodiments, the antibody, or antigen binding fragment thereof, comprises a VH sequence set forth in SEQ ID NO: 434 and a VL sequence set forth in SEQ ID NO: 502.

In certain embodiments, the antibody, or antigen binding fragment thereof, comprises a VH sequence set forth in SEQ ID NO: 435 and a VL sequence set forth in SEQ ID NO: 502.

In certain embodiments, the antibody, or antigen binding fragment thereof, comprises a VH sequence set forth in SEQ ID NO: 436 and a VL sequence set forth in SEQ ID NO: 502.

In certain embodiments, the antibody, or antigen binding fragment thereof, comprises a VH sequence set forth in SEQ ID NO: 437 and a VL sequence set forth in SEQ ID NO: 502.

In certain embodiments, the antibody, or antigen binding fragment thereof, comprises a VH sequence set forth in SEQ ID NO: 438 and a VL sequence set forth in SEQ ID NO: 503.

In certain embodiments, the antibody, or antigen binding fragment thereof, comprises a VH sequence set forth in SEQ ID NO: 439 and a VL sequence set forth in SEQ ID NO: 502.

In certain embodiments, the antibody, or antigen binding fragment thereof, comprises a VH sequence set forth in SEQ ID NO: 440 and a VL sequence set forth in SEQ ID NO: 504.

In certain embodiments, the antibody, or antigen binding fragment thereof, comprises a VH sequence set forth in SEQ ID NO: 441 and a VL sequence set forth in SEQ ID NO: 505.

In certain embodiments, the antibody, or antigen binding fragment thereof, comprises a VH sequence set forth in SEQ ID NO: 442 and a VL sequence set forth in SEQ ID NO: 506.

In certain embodiments, the antibody, or antigen binding fragment thereof, comprises a VH sequence set forth in SEQ ID NO: 443 and a VL sequence set forth in SEQ ID NO: 507.

In certain embodiments, the antibody, or antigen binding fragment thereof, comprises a VH sequence set forth in SEQ ID NO: 443 and a VL sequence set forth in SEQ ID NO: 508.

In certain embodiments, the antibody, or antigen binding fragment thereof, comprises a VH sequence set forth in SEQ ID NO: 444 and a VL sequence set forth in SEQ ID NO: 506.

In certain embodiments, the antibody, or antigen binding fragment thereof, comprises a VH sequence set forth in SEQ ID NO: 445 and a VL sequence set forth in SEQ ID NO: 509.

In certain embodiments, the antibody, or antigen binding fragment thereof, comprises a VH sequence set forth in SEQ ID NO: 446 and a VL sequence set forth in SEQ ID NO: 510.

In certain embodiments, the antibody, or antigen binding fragment thereof, comprises a VH sequence set forth in SEQ ID NO: 447 and a VL sequence set forth in SEQ ID NO: 506.

In certain embodiments, the antibody, or antigen binding fragment thereof, comprises a VH sequence set forth in SEQ ID NO: 445 and a VL sequence set forth in SEQ ID NO: 511.

In certain embodiments, the antibody, or antigen binding fragment thereof, comprises a VH sequence set forth in SEQ ID NO: 448 and a VL sequence set forth in SEQ ID NO: 506.

In certain embodiments, the antibody, or antigen binding fragment thereof, comprises a VH sequence set forth in SEQ ID NO: 449 and a VL sequence set forth in SEQ ID NO: 502.

In certain embodiments, the antibody, or antigen binding fragment thereof, comprises a VH sequence set forth in SEQ ID NO: 449 and a VL sequence set forth in SEQ ID NO: 506.

In certain embodiments, the antibody, or antigen binding fragment thereof, comprises a VH sequence set forth in SEQ ID NO: 450 and a VL sequence set forth in SEQ ID NO: 506.

In certain embodiments, the antibody, or antigen binding fragment thereof, comprises a VH sequence set forth in SEQ ID NO: 445 and a VL sequence set forth in SEQ ID NO: 512.

In certain embodiments, the antibody, or antigen binding fragment thereof, comprises a VH sequence set forth in SEQ ID NO: 449 and a VL sequence set forth in SEQ ID NO: 513.

In certain embodiments, the antibody, or antigen binding fragment thereof, comprises a VH sequence set forth in SEQ ID NO: 445 and a VL sequence set forth in SEQ ID NO: 514.

In certain embodiments, the antibody, or antigen binding fragment thereof, comprises a VH sequence set forth in SEQ ID NO: 445 and a VL sequence set forth in SEQ ID NO: 515.

In certain embodiments, the antibody, or antigen binding fragment thereof, comprises a VH sequence set forth in SEQ ID NO: 451 and a VL sequence set forth in SEQ ID NO: 514.

In certain embodiments, the antibody, or antigen binding fragment thereof, comprises a VH sequence set forth in SEQ ID NO: 451 and a VL sequence set forth in SEQ ID NO: 506.

In certain embodiments, the antibody, or antigen binding fragment thereof, comprises a VH sequence set forth in SEQ ID NO: 452 and a VL sequence set forth in SEQ ID NO: 506.

In certain embodiments, the antibody, or antigen binding fragment thereof, comprises a VH sequence set forth in SEQ ID NO: 438 and a VL sequence set forth in SEQ ID NO: 506.

In certain embodiments, the antibody, or antigen binding fragment thereof, comprises a VH sequence set forth in SEQ ID NO: 453 and a VL sequence set forth in SEQ ID NO: 512.

In certain embodiments, the antibody, or antigen binding fragment thereof, comprises a VH sequence set forth in SEQ ID NO: 454 and a VL sequence set forth in SEQ ID NO: 490.

In certain embodiments, the antibody, or antigen binding fragment thereof, comprises a VH sequence set forth in SEQ ID NO: 455 and a VL sequence set forth in SEQ ID NO: 516.

In certain embodiments, the antibody, or antigen binding fragment thereof, comprises a VH sequence set forth in SEQ ID NO: 456 and a VL sequence set forth in SEQ ID NO: 517.

In certain embodiments, the antibody, or antigen binding fragment thereof, comprises a VH sequence set forth in SEQ ID NO: 457 and a VL sequence set forth in SEQ ID NO: 518.

In certain embodiments, the antibody, or antigen binding fragment thereof, comprises a VH sequence set forth in SEQ ID NO: 458 and a VL sequence set forth in SEQ ID NO: 492.

In certain embodiments, the antibody, or antigen binding fragment thereof, comprises a VH sequence set forth in SEQ ID NO: 459 and a VL sequence set forth in SEQ ID NO: 519.

In certain embodiments, the antibody, or antigen binding fragment thereof, comprises a VH sequence set forth in SEQ ID NO: 460 and a VL sequence set forth in SEQ ID NO: 520.

In certain embodiments, the antibody, or antigen binding fragment thereof, comprises a VH sequence set forth in SEQ ID NO: 461 and a VL sequence set forth in SEQ ID NO: 521.

In certain embodiments, the antibody, or antigen binding fragment thereof, comprises a VH sequence set forth in SEQ ID NO: 461 and a VL sequence set forth in SEQ ID NO: 522.

In certain embodiments, the antibody, or antigen binding fragment thereof, comprises a VH sequence set forth in SEQ ID NO: 461 and a VL sequence set forth in SEQ ID NO: 522.

In certain embodiments, the antibody, or antigen binding fragment thereof, comprises a VH sequence set forth in SEQ ID NO: 462 and a VL sequence set forth in SEQ ID NO: 523.

In certain embodiments, the antibody, or antigen binding fragment thereof, comprises a VH sequence set forth in SEQ ID NO: 463 and a VL sequence set forth in SEQ ID NO: 523.

In certain embodiments, the antibody, or antigen binding fragment thereof, comprises a VH sequence set forth in SEQ ID NO: 464 and a VL sequence set forth in SEQ ID NO: 524.

In certain embodiments, the antibody, or antigen binding fragment thereof, comprises a VH sequence set forth in SEQ ID NO: 465 and a VL sequence set forth in SEQ ID NO: 525.

In certain embodiments, the antibody, or antigen binding fragment thereof, comprises a VH sequence set forth in SEQ ID NO: 466 and a VL sequence set forth in SEQ ID NO: 526.

In certain embodiments, the antibody, or antigen binding fragment thereof, comprises a VH sequence set forth in SEQ ID NO: 467 and a VL sequence set forth in SEQ ID NO: 527.

In certain embodiments, the antibody, or antigen binding fragment thereof, comprises a VH sequence set forth in SEQ ID NO: 468 and a VL sequence set forth in SEQ ID NO: 528.

In certain embodiments, the antibody, or antigen binding fragment thereof, comprises a VH sequence set forth in SEQ ID NO: 469 and a VL sequence set forth in SEQ ID NO: 529.

In certain embodiments, the antibody, or antigen binding fragment thereof, comprises a VH sequence set forth in SEQ ID NO: 470 and a VL sequence set forth in SEQ ID NO: 528.

In certain embodiments, the antibody, or antigen binding fragment thereof, comprises a VH sequence set forth in SEQ ID NO: 471 and a VL sequence set forth in SEQ ID NO: 530.

In certain embodiments, the antibody, or antigen binding fragment thereof, comprises a VH sequence set forth in SEQ ID NO: 472 and a VL sequence set forth in SEQ ID NO: 531.

In certain embodiments, the antibody, or antigen binding fragment thereof, comprises a VH sequence set forth in SEQ ID NO: 473 and a VL sequence set forth in SEQ ID NO: 532

In certain embodiments, the antibody, or antigen binding fragment thereof, comprises a VH sequence set forth in SEQ ID NO: 474 and a VL sequence set forth in SEQ ID NO: 533.

In certain embodiments, the antibody, or antigen binding fragment thereof, comprises a VH sequence set forth in SEQ ID NO: 475 and a VL sequence set forth in SEQ ID NO: 534.

In certain embodiments, the antibody, or antigen binding fragment thereof, comprises a VH sequence set forth in SEQ ID NO: 476 and a VL sequence set forth in SEQ ID NO: 535.

In certain embodiments, the antibody, or antigen binding fragment thereof, comprises a VH sequence set forth in SEQ ID NO: 477 and a VL sequence set forth in SEQ ID NO: 536.

In certain embodiments, the antibody, or antigen binding fragment thereof, comprises a VH sequence set forth in SEQ ID NO: 478 and a VL sequence set forth in SEQ ID NO: 536.

In certain embodiments, the antibody, or antigen binding fragment thereof, comprises a VH sequence set forth in SEQ ID NO: 479 and a VL sequence set forth in SEQ ID NO: 537.

In certain embodiments, the antibody, or antigen binding fragment thereof, comprises a VH sequence set forth in SEQ ID NO: 480 and a VL sequence set forth in SEQ ID NO: 538.

In certain embodiments, the antibody, or antigen binding fragment thereof, comprises a VH sequence set forth in SEQ ID NO: 481 and a VL sequence set forth in SEQ ID NO: 539.

In certain embodiments, the antibody, or antigen binding fragment thereof, comprises a VH sequence set forth in SEQ ID NO: 482 and a VL sequence set forth in SEQ ID NO: 539.

In certain embodiments, the antibody, or antigen binding fragment thereof, comprises a VH sequence set forth in SEQ ID NO: 483 and a VL sequence set forth in SEQ ID NO: 539.

In certain embodiments, the antibody, or antigen binding fragment thereof, comprises a VH sequence set forth in SEQ ID NO: 484 and a VL sequence set forth in SEQ ID NO: 540.

In certain embodiments, the antibody, or antigen binding fragment thereof, comprises a VH sequence set forth in SEQ ID NO: 485 and a VL sequence set forth in SEQ ID NO: 541.

In certain embodiments, the antibody, or antigen binding fragment thereof, comprises a VH sequence set forth in SEQ ID NO: 486 and a VL sequence set forth in SEQ ID NO: 542.

In certain embodiments, the antibody, or antigen binding fragment thereof, comprises a VH sequence set forth in SEQ ID NO: 487 and a VL sequence set forth in SEQ ID NO: 543.

In certain embodiments, the antibody, or antigen binding fragment thereof, comprises a VH sequence set forth in SEQ ID NO: 488 and a VL sequence set forth in SEQ ID NO: 544.

In certain embodiments, the antibody, or antigen binding fragment thereof, comprises a VH sequence set forth in SEQ ID NO: 818 and a VL sequence set forth in SEQ ID NO: 825.

In certain embodiments, the antibody, or antigen binding fragment thereof, comprises a VH sequence set forth in SEQ ID NO: 819 and a VL sequence set forth in SEQ ID NO: 826.

In certain embodiments, the antibody, or antigen binding fragment thereof, comprises a VH sequence set forth in SEQ ID NO: 820 and a VL sequence set forth in SEQ ID NO: 827.

In certain embodiments, the antibody, or antigen binding fragment thereof, comprises a VH sequence set forth in SEQ ID NO: 821 and a VL sequence set forth in SEQ ID NO: 828.

In certain embodiments, the antibody, or antigen binding fragment thereof, comprises a VH sequence set forth in SEQ ID NO: 822 and a VL sequence set forth in SEQ ID NO: 829.

In certain embodiments, the antibody, or antigen binding fragment thereof, comprises a VH sequence set forth in SEQ ID NO: 823 and a VL sequence set forth in SEQ ID NO: 830.

In certain embodiments, the antibody, or antigen binding fragment thereof, comprises a VH sequence set forth in SEQ ID NO: 824 and a VL sequence set forth in SEQ ID NO: 831.

In certain embodiments, the antibody, or antigen binding fragment thereof, comprises a VH sequence set forth in SEQ ID NO: 841 and a VL sequence set forth in SEQ ID NO: 842.

In certain embodiments, an antibody, or antigen binding fragment thereof, comprises any of the VH and VL combinations described above and further comprises a heavy chain comprising a human IgG sequence selected from a sequence set forth in any one of SEQ ID NOs: 637-737 and 913-1012 or a sequence having at least about 80%, 90%, 91%, 92%, 93%, 94%, 95%, 96%, 97%, 98% or 99% identity thereto. In certain embodiments, an antibody comprises any of the VH and VL combinations described above and further comprises a heavy chain comprising a heavy chain sequence set forth in SEQ ID NO: 651 or SEQ ID NO: 927 or a sequence having at least about 80%, 90%, 91%, 92%, 93%, 94%, 95%, 96%, 97%, 98% or 99% identity thereto. In certain embodiments, an antibody comprises any of the VH and VL combinations described above and further comprises a heavy chain comprising a heavy chain sequence set forth in SEQ ID NO: 651 or a sequence having at least about 80%, 90%, 91%, 92%, 93%, 94%, 95%, 96%, 97%, 98% or 99% identity thereto. In certain embodiments, an antibody comprises any of the VH and VL combinations described above and further comprises a heavy chain comprising a heavy chain sequence set forth in SEQ ID NO: 927 or a sequence having at least about 80%, 90%, 91%, 92%, 93%, 94%, 95%, 96%, 97%, 98% or 99% identity thereto. In certain embodiments, an antibody comprises any of the VH and VL combinations described above and further comprises a human light chain sequence set forth in SEQ ID NO: 738 or a sequence having at least about 80%, 90%, 91%, 92%, 93%, 94%, 95%, 96%, 97%, 98% or 99% identity thereto. In certain embodiments, an antibody comprises any of the VH and VL combinations described above and further comprises a heavy chain sequence set forth in SEQ ID NO: 651 or a sequence having at least about 80%, 90%, 91%, 92%, 93%, 94%, 95%, 96%, 97%, 98% or 99% identity thereto and a human light chain sequence set forth in SEQ ID NO: 738 or a sequence having at least about 80%, 90%, 91%, 92%, 93%, 94%, 95%, 96%, 97%, 98% or 99% identity thereto. In certain embodiments, an antibody comprises any of the VH and VL combinations described above and further comprises a heavy chain sequence set forth in SEQ ID NO: 927 or a sequence having at least about 80%, 90%, 91%, 92%, 93%, 94%, 95%, 96%, 97%, 98% or 99% identity thereto and a human light chain sequence set forth in SEQ ID NO: 738 or a sequence having at least about 80%, 90%, 91%, 92%, 93%, 94%, 95%, 96%, 97%, 98% or 99% identity thereto.

In some embodiments, an antibody, or antigen binding fragment thereof, provided herein comprises a CDR-H1 comprising an amino acid sequence set forth in any one of SEQ ID NOs: 1, 22, and 44; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 258, 286, and 311; a CDR-L2 comprising an amino acid sequence set forth in any one of SEQ ID NOs: 335 and 359; a CDR-L3 comprising the sequence set forth in any one of SEQ ID NOs: 368 and 394, a heavy chain sequence set forth in SEQ ID NO: 651 or SEQ ID NO: 927 or a sequence having at least about 80%, 90%, 91%, 92%, 93%, 94%, 95%, 96%, 97%, 98% or 99% identity thereto and a human light chain sequence set forth in SEQ ID NO: 738 or a sequence having at least about 80%, 90%, 91%, 92%, 93%, 94%, 95%, 96%, 97%, 98% or 99% identity thereto.

In certain embodiments, an antibody, or antigen binding fragment thereof, comprises a VH comprising SEQ ID NO: 419 or a sequence having at least about 80%, 90%, 91%, 92%, 93%, 94%, 95%, 96%, 97%, 98% or 99% identity thereto, a VL comprising SEQ ID NO: 489 or a sequence having at least about 80%, 90%, 91%, 92%, 93%, 94%, 95%, 96%, 97%, 98% or 99% identity thereto, a heavy chain sequence set forth in SEQ ID NO: 651 or SEQ ID NO: 927 or a sequence having at least about 80%, 90%, 91%, 92%, 93%, 94%, 95%, 96%, 97%, 98% or 99% identity thereto and a human light chain sequence set forth in SEQ ID NO: 738 or a sequence having at least about 80%, 90%, 91%, 92%, 93%, 94%, 95%, 96%, 97%, 98% or 99% identity thereto.

In some embodiments, an antibody, or antigen binding fragment thereof, provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 752, 756, and 761; a CDR-H3 comprising an amino acid sequence set forth in any one of SEQ ID NOs: 767, 773, and 779, a CDR-L1 comprising an amino acid sequence set forth in any one of SEQ ID NOs: 785, 790, and 795; a CDR-L2 comprising an amino acid sequence set forth in any one of SEQ ID NOs: 800 and 805; a CDR-L3 comprising an amino acid sequence set forth in any one of SEQ ID NOs: 810 and 816, a heavy chain sequence set forth in SEQ ID NO: 651 or SEQ ID NO: 927 or a sequence having at least about 80%, 90%, 91%, 92%, 93%, 94%, 95%, 96%, 97%, 98% or 99% identity thereto and a human light chain sequence set forth in SEQ ID NO: 738 or a sequence having at least about 80%, 90%, 91%, 92%, 93%, 94%, 95%, 96%, 97%, 98% or 99% identity thereto.

In certain embodiments, an antibody, or antigen binding fragment thereof, comprises a VH comprising SEQ ID NO: 823 or a sequence having at least about 80%, 90%, 91%, 92%, 93%, 94%, 95%, 96%, 97%, 98% or 99% identity thereto, a VL comprising SEQ ID NO: 830 or a sequence having at least about 80%, 90%, 91%, 92%, 93%, 94%, 95%, 96%, 97%, 98% or 99% identity thereto, a heavy chain sequence set forth in SEQ ID NO: 651 or SEQ ID NO: 927 or a sequence having at least about 80%, 90%, 91%, 92%, 93%, 94%, 95%, 96%, 97%, 98% or 99% identity thereto and a human light chain sequence set forth in SEQ ID NO: 738 or a sequence having at least about 80%, 90%, 91%, 92%, 93%, 94%, 95%, 96%, 97%, 98% or 99% identity thereto.

In some embodiments, an antibody, or antigen binding fragment thereof, provided herein comprises a CDR-H1 comprising an amino acid sequence set forth in any one of SEQ ID NOs: 741, 744, and 747; a CDR-H2 comprising an amino acid sequence set forth in any one of SEQ ID NOs: 753, 757, and 762; a CDR-H3 comprising an amino acid sequence set forth in any one of SEQ ID NOs: 768, 774, and 780, a CDR-L1 comprising an amino acid sequence set forth in any one of SEQ ID NOs: 786, 791, and 796; a CDR-L2 comprising an amino acid sequence set forth in any one of SEQ ID NOs: 801 and 360; a CDR-L3 comprising an amino acid sequence set forth in any one of SEQ ID NOs: 811 and 817, a heavy chain sequence set forth in SEQ ID NO: 651 or SEQ ID NO: 927 or a sequence having at least about 80%, 90%, 91%, 92%, 93%, 94%, 95%, 96%, 97%, 98% or 99% identity thereto and a human light chain sequence set forth in SEQ ID NO: 738 or a sequence having at least about 80%, 90%, 91%, 92%, 93%, 94%, 95%, 96%, 97%, 98% or 99% identity thereto.

In certain embodiments, an antibody, or antigen binding fragment thereof, comprises a VH comprising SEQ ID NO: 824 or a sequence having at least about 80%, 90%, 91%, 92%, 93%, 94%, 95%, 96%, 97%, 98% or 99% identity thereto, a VL comprising SEQ ID NO: 831 or a sequence having at least about 80%, 90%, 91%, 92%, 93%, 94%, 95%, 96%, 97%, 98% or 99% identity thereto, a heavy chain sequence set forth in SEQ ID NO: 651 or SEQ ID NO: 927 or a sequence having at least about 80%, 90%, 91%, 92%, 93%, 94%, 95%, 96%, 97%, 98% or 99% identity thereto and a human light chain sequence set forth in SEQ ID NO: 738 or a sequence having at least about 80%, 90%, 91%, 92%, 93%, 94%, 95%, 96%, 97%, 98% or 99% identity thereto.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 234, a CDR-L1 comprising an amino acid sequence set forth in any one of SEQ ID NOs: 260, 288, 313; a CDR-L2 comprising an amino acid sequence set forth in any one of SEQ ID NOs: 347 and 365; a CDR-L3 comprising an amino acid sequence set forth in any one of SEQ ID NOs: 378 and 404, a heavy chain sequence set forth in SEQ ID NO: 651 or SEQ ID NO: 927 or a sequence having at least about 80%, 90%, 91%, 92%, 93%, 94%, 95%, 96%, 97%, 98% or 99% identity thereto and a human light chain sequence set forth in SEQ ID NO: 738 or a sequence having at least about 80%, 90%, 91%, 92%, 93%, 94%, 95%, 96%, 97%, 98% or 99% identity thereto.

In certain embodiments, an antibody, or antigen binding fragment thereof, comprises a VH comprising SEQ ID NO: 841 or a sequence having at least about 80%, 90%, 91%, 92%, 93%, 94%, 95%, 96%, 97%, 98% or 99% identity thereto, a VL comprising SEQ ID NO: 842 or a sequence having at least about 80%, 90%, 91%, 92%, 93%, 94%, 95%, 96%, 97%, 98% or 99% identity thereto, a heavy chain sequence set forth in SEQ ID NO: 651 or SEQ ID NO: 927 or a sequence having at least about 80%, 90%, 91%, 92%, 93%, 94%, 95%, 96%, 97%, 98% or 99% identity thereto and a human light chain sequence set forth in SEQ ID NO: 738 or a sequence having at least about 80%, 90%, 91%, 92%, 93%, 94%, 95%, 96%, 97%, 98% or 99% identity thereto.

Although the antibody may be produced using a polynucleotide sequence that encodes a constant heavy chain region containing a C-terminal lysine (e.g., a sequence encoding any one of SEQ ID NOs: 913-1012), the C-terminal lysine may be cleaved off during manufacture or after administration (resulting in, e.g., an antibody containing a constant heavy chain sequence of any one of SEQ ID NOs: 637-737). Accordingly, any of the antibodies or antigen binding fragments thereof described above may comprise a human IgG sequence containing a C-terminal lysine (e.g., any one of SEQ ID NOs: 913-1012), a human IgG sequence lacking a C-terminal lysine (e.g., any one of SEQ ID NOs: 637-737), or a mixture thereof (e.g., a mixture of the same heavy chain constant region sequence with and without a C-terminal lysine, such as a mixture of SEQ ID NO: 927 and SEQ ID NO: 651).

### CDRs

In some embodiments, disclosed herein is an antibody, or antigen binding fragment thereof, comprising 1, 2, 3, 4, 5, or 6 of the CDRs of TABLE 3. In some embodiments, disclosed herein is an antibody comprising 6 of the Kabat CDRs of TABLE 3 or TABLE 4, 6 of the Chothia CDRs of TABLE 3 or TABLE 4, or 6 of the IMGT CDRs of TABLE 3 or TABLE 4. In some embodiments, the antibody, or an antigen binding fragment thereof, comprises 6 of the Kabat CDRs, 6 of the Chothia CDRs, or 6 of the IMGT CDRs from a single row of TABLE 3 (e.g., 6 CDRs from the same antibody).

In some embodiments, disclosed herein is an antibody, or an antigen binding fragment thereof, comprising 1, 2, 3, 4, 5, or 6 of the CDRs of an antibody provided in PCT Publication No. WO2024227174, incorporated by reference herein in its entirety, such as 1, 2, 3, 4, 5, or 6 of the CDRs in Table 2 in PCT Publication No. WO2024227174. In some embodiments, disclosed herein is an antibody, or an antigen binding fragment thereof, comprising 1, 2, 3, 4, 5, or 6 of the Kabat CDRs of an antibody, or an antigen binding fragment thereof, provided in PCT Publication No. WO2024227174. In some embodiments, disclosed herein is an antibody, or an antigen binding fragment thereof, comprising 1, 2, 3, 4, 5, or 6 of the Chothia CDRs of an antibody, or an antigen binding fragment thereof, provided in PCT Publication No. WO2024227174. In some embodiments, disclosed herein is an antibody, or an antigen binding fragment thereof, comprising 1, 2, 3, 4, 5, or 6 of the IMGT CDRs of an antibody provided in PCT Publication No. WO2024227174. In some embodiments, the antibody, or an antigen binding fragment thereof, comprises 6 of the Kabat CDRs, 6 of the Chothia CDRs, or 6 of the IMGT CDRs from a single row of Table 2 in PCT Publication No. WO2024227174 (e.g., 6 CDRs from the same antibody).

In some embodiments, an antibody, or antigen binding fragment thereof, provided herein comprises one to three CDRs of a VH domain selected from any one of SEQ ID NOs: 419-488, 818-824, and 841. In some embodiments, an antibody, or antigen binding fragment thereof, provided herein comprises two to three CDRs of a VH domain selected from any one of SEQ ID NOs: 419-488, 818-824, and 841. In some embodiments, an antibody, or antigen binding fragment thereof, provided herein comprises three CDRs of a VH domain selected from any one of SEQ ID NOs: 419-488, 818-824, and 841. In some aspects, the CDRs are exemplary CDRs. In some aspects, the CDRs are Kabat CDRs. In some aspects, the CDRs are Chothia CDRs. In some aspects, the CDRs are IMGT CDRs. In some aspects, the CDRs are AbM CDRs. In some aspects, the CDRs are Contact CDRs.

In some embodiments, the CDRs are CDRs having at least about 80%, 90%, 91%, 92%, 93%, 94%, 95%, 96%, 97%, 98%, 99% or 100% identity to a CDR-H1, CDR-H2, or CDR-H3 selected from SEQ ID NOs: 1-257 and 740-780. In some embodiments, the CDR-H1 is a CDR-H1 of a VH domain selected from any one of SEQ ID NOs: 419-488, 818-824, and 841, with up to 1, 2, 3, 4, or 5 amino acid substitutions. In some embodiments, the CDR-H2 is a CDR-H2 of a VH domain selected from any one of SEQ ID NOs: 419-488, 818-824, and 841, with up to 1, 2, 3, 4, 5, 6, 7, or 8 amino acid substitutions. In some embodiments, the CDR-H3 is a CDR-H3 of a VH domain selected from any one of SEQ ID NOs: 419-488, 818-824, and 841, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises one to three CDRs of a VL domain selected from any one of SEQ ID NOs: 489-544, 825-831, and 842. In some embodiments, an antibody, or antigen binding fragment thereof, provided herein comprises two to three CDRs of a VL domain selected from any one of SEQ ID NOs: 489-544, 825-831, and 842. In some embodiments, an antibody, or antigen binding fragment thereof, provided herein comprises three CDRs of a VL domain selected from any one of SEQ ID NOs: 489-544, 825-831, and 842. In some aspects, the CDRs are exemplary CDRs. In some aspects, the CDRs are Kabat CDRs. In some aspects, the CDRs are Chothia CDRs. In some aspects, the CDRs are IMGT CDRs. In some aspects, the CDRs are AbM CDRs. In some aspects, the CDRs are Contact CDRs.

In some embodiments, the CDRs are CDRs having at least about 80%, 90%, 91%, 92%, 93%, 94%, 95%, 96%, 97%, 98%, 99% or 100% identity to a CDR-L1, CDR-L2, or CDR-L3 selected from SEQ ID NOs: 258-393 and 781-811, In some embodiments, the CDR-L1 is a CDR-L1 of a VL domain selected from any one of SEQ ID NOs: 489-544, 825-831, and 842, with up to 1, 2, 3, 4, or 5 amino acid substitutions. In some embodiments, the CDR-L2 is a CDR-L2 of a VL domain selected from any one of SEQ ID NOs: 489-544, 825-831, and 842, with up to 1, 2, 3, 4, 5, 6, 7, or 8 amino acid substitutions. In some embodiments, the CDR-L3 is a CDR-L3 of a VL domain selected from any one of SEQ ID NOs: 489-544, 825-831, and 842, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises one to three CDRs of a VH domain selected from any one of SEQ ID NOs: 419-488, 818-824, and 841 and one to three CDRs of a VL domain selected from any one of SEQ ID NOs: 489-544, 825-831, and 842. In some embodiments, an antibody, or antigen binding fragment thereof, provided herein comprises two to three CDRs of a VH domain selected from any one of SEQ ID NOs: 419-488, 818-824, and 841 and two to three CDRs of a VL domain selected from any one of SEQ ID NOs: 489-544, 825-831, and 842. In some embodiments, an antibody, or antigen binding fragment thereof, provided herein comprises three CDRs of a VH domain selected from any one of SEQ ID NOs: 419-488, 818-824, and 841 and three CDRs of a VL domain selected from any one of SEQ ID NOs: 489-544, 825-831, and 842. In some aspects, the CDRs are exemplary CDRs. In some aspects, the CDRs are Kabat CDRs. In some aspects, the CDRs are Chothia CDRs. In some aspects, the CDRs are IMGT CDRs. In some aspects, the CDRs are AbM CDRs. In some aspects, the CDRs are Contact CDRs.

In some embodiments, an antibody, or antigen binding fragment thereof, provided herein comprises a CDR-H3 selected from any one of SEQ ID NOs: 175-257 and 763-780. In some aspects, the CDR-H3 has at least about 80%, 90%, 91%, 92%, 93%, 94%, 95%, 96%, 97%, 98% or 99% identity to a CDR-H3 selected from any one of SEQ ID NOs: 175-257 and 763-780. In some embodiments, the CDR-H3 is a CDR-H3 selected from any one of SEQ ID NOs: 175-257 and 763-780, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises a CDR-H1 selected from any one of SEQ ID NOs: 1-69, 740-747, and 840. In some aspects, the CDR-H1 has at least about 80%, 90%, 91%, 92%, 93%, 94%, 95%, 96%, 97%, 98% or 99% identity to a CDR-H1 selected from any one of SEQ ID NOs: 1-69, 740-747, and 840. In some embodiments, the CDR-H1 is a CDR-H1 selected from any one of SEQ ID NOs: 1-69, 740-747, and 840, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises a CDR-H2 selected from any one of SEQ ID NOs: 70-174 and 748-762. In some aspects, the CDR-H2 has at least about 80%, 90%, 91%, 92%, 93%, 94%, 95%, 96%, 97%, 98% or 99% identity to a CDR-H2 H2 selected from any one of SEQ ID NOs: 70-174 and 748-762. In some embodiments, the CDR-H2 is a CDR-H2 selected from any one of SEQ ID NOs: 70-174 and 748-762, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises a CDR-L3 selected from any one of SEQ ID NOs: 268-393 and 806-811. In some aspects, the CDR-L3 has at least about 80%, 90%, 91%, 92%, 93%, 94%, 95%, 96%, 97%, 98% or 99% identity to a CDR-L3 selected from any one of SEQ ID NOs: 268-393 and 806-811. In some embodiments, the CDR-L3 is a CDR-L3 selected from any one of SEQ ID NOs: 268-393 and 806-811, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises a CDR-L2 selected from any one of SEQ ID NOs: 335-367 and 797-805. In some aspects, the CDR-L2 has at least about 80%, 90%, 91%, 92%, 93%, 94%, 95%, 96%, 97%, 98% or 99% identity to a CDR-L2 selected from any one of SEQ ID NOs: 335-367 and 797-805. In some embodiments, the CDR-L2 is a CDR-L2 selected from any one of SEQ ID NOs: 335-367 and 797-805, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises a CDR-L1 selected from any one of SEQ ID NOs: 258-334 and 781-796. In some aspects, the CDR-L1 has at least about 80%, 90%, 91%, 92%, 93%, 94%, 95%, 96%, 97%, 98% or 99% identity to a CDR-L1 selected from any one of SEQ ID NOs: 258-334 and 781-796. In some embodiments, the CDR-L1 is a CDR-L1 selected from any one of SEQ ID NOs: 258-334 and 781-796, with up to 1, 2, 3, 4, 5, 6, 7, or 8 amino acid substitutions. In some aspects, the amino acid substitutions are conservative amino acid substitutions. In some embodiments, the antibodies described in this paragraph are referred to herein as “variants.” In some embodiments, such variants are derived from a sequence provided herein, for example, by affinity maturation, site directed mutagenesis, random mutagenesis, or any other method known in the art or described herein. In some embodiments, such variants are not derived from a sequence provided herein and may, for example, be isolated de novo according to the methods provided herein for obtaining antibodies.

In some embodiments, an antibody, or antigen binding fragment thereof, provided herein comprises a CDR-H3 selected from any one of SEQ ID NOs: 175-257 and 763-780, a CDR-H2 selected from any one of SEQ ID NOs: 70-174 and 748-762, a CDR-H1 selected from any one of SEQ ID NOs: 1-69 and 740-747, a CDR-L3 selected from any one of SEQ ID NOs: 268-393 and 806-811, a CDR-L2 selected from any one of SEQ ID NOs: 335-367 and 797-805, and a CDR-L1 selected from any one of SEQ ID NOs: 258-334 and 781-796. In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175-257 and 763-780, the CDR-H2 has at least about 80%, 90%, 91%, 92%, 93%, 94%, 95%, 96%, 97%, 98% or 99% identity to a CDR-H2 selected from any one of SEQ ID NOs: 70-174 and 748-762, the CDR-H1 has at least about 80%, 90%, 91%, 92%, 93%, 94%, 95%, 96%, 97%, 98% or 99% identity to a CDR-H1 selected from any one of SEQ ID NOs: 1-69 and 740-747, the CDR-L3 has at least about 80%, 90%, 91%, 92%, 93%, 94%, 95%, 96%, 97%, 98% or 99% identity to a CDR-L3 selected from any one of SEQ ID NOs: 268-393 and 806-811, the CDR-L2 has at least about 80%, 90%, 91%, 92%, 93%, 94%, 95%, 96%, 97%, 98% or 99% identity to a CDR-L2 selected from any one of SEQ ID NOs: 335-367 and 797-805, and the CDR-L1 has at least about 80%, 90%, 91%, 92%, 93%, 94%, 95%, 96%, 97%, 98% or 99% identity to a CDR-L1 selected from any one of SEQ ID NOs: 258-334 and 781-796. In some embodiments, the CDR-H3 is a CDR-H3 selected from any one of SEQ ID NOs: 175-257 and 763-780, with up to 1, 2, 3, 4, 5, 6, 7, or 8 amino acid substitutions; the CDR-H2 is a CDR-H2 selected from any one of SEQ ID NOs: 70-174 and 748-762, with up to 1, 2, 3, 4, 5, 6, 7, or 8 amino acid substitutions; the CDR-H1 is a CDR-H1 selected from any one of SEQ ID NOs: 1-69 and 740-747, with up to 1, 2, 3, 4, or 5 amino acid substitutions; the CDR-L3 is a CDR-L3 selected from any one of SEQ ID NOs: 268-393 and 806-811, with up to 1, 2, 3, 4, or 5 amino acid substitutions; the CDR-L2 is a CDR-L2 selected from any one of SEQ ID NOs: 335-367 and 797-805, with up to 1, 2, 3, or 4 amino acid substitutions; and the CDR-L1 is a CDR-L1 selected from any one of SEQ ID NOs: 258-334 and 781-796, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 1, 22, and 44; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 258, 286, and 311; a CDR-L2 comprising an amino acid sequence set forth in any one of SEQ ID NOs: 335 and 359; and a CDR-L3 comprising the sequence set forth in any one of SEQ ID NOs: 368 and 394.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175, 203, and 223, the CDR-H2 has at least about 80%, 90%, 91%, 92%, 93%, 94%, 95%, 96%, 97%, 98% or 99% identity to a CDR-H2 selected from any one of SEQ ID NOs: 70, 111, and 142, the CDR-H1 has at least about 80%, 90%, 91%, 92%, 93%, 94%, 95%, 96%, 97%, 98% or 99% identity to a CDR-H1 selected from any one of SEQ ID NOs: 1, 22, and 44, the CDR-L3 has at least about 80%, 90%, 91%, 92%, 93%, 94%, 95%, 96%, 97%, 98% or 99% identity to a CDR-L3 selected from any one of SEQ ID NOs: 368 and 394, the CDR-L2 has at least about 80%, 90%, 91%, 92%, 93%, 94%, 95%, 96%, 97%, 98% or 99% identity to a CDR-L2 selected from any one of SEQ ID NOs: 335 and 359, and the CDR-L1 has at least about 80%, 90%, 91%, 92%, 93%, 94%, 95%, 96%, 97%, 98% or 99% identity to a CDR-L1 selected from any one of SEQ ID NOs: 258, 286, and 311. In some embodiments, the CDR-H3 is a CDR-H3 selected from any one of SEQ ID NOs: 175, 203, and 223, with up to 1, 2, 3, 4, 5, 6, 7, or 8 amino acid substitutions; the CDR-H2 is a CDR-H2 selected from any one of SEQ ID NOs: 70, 111, and 142, with up to 1, 2, 3, 4, 5, 6, 7, or 8 amino acid substitutions; the CDR-H1 is a CDR-H1 selected from any one of SEQ ID NOs: 1, 22, and 44, with up to 1, 2, 3, 4, or 5 amino acid substitutions; the CDR-L3 is a CDR-L3 selected from any one of SEQ ID NOs: 368 and 394, with up to 1, 2, 3, 4, or 5 amino acid substitutions; the CDR-L2 is a CDR-L2 selected from any one of SEQ ID NOs: 335 and 359, with up to 1, 2, 3, or 4 amino acid substitutions; and the CDR-L1 is a CDR-L1 selected from any one of SEQ ID NOs: 258, 286, and 311, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 1, 22, and 44; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 258, 286, and 311; a CDR-L2 comprising an amino acid sequence set forth in any one of SEQ ID NOs: 335 and 359; and a CDR-L3 comprising an amino acid sequence set forth in any one of SEQ ID NOs: 368 and 394.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 176, 204, and 224, the CDR-H2 has at least about 80%, 90%, 91%, 92%, 93%, 94%, 95%, 96%, 97%, 98% or 99% identity to a CDR-H2 selected from any one of SEQ ID NOs: 70, 111, and 142, the CDR-H1 has at least about 80%, 90%, 91%, 92%, 93%, 94%, 95%, 96%, 97%, 98% or 99% identity to a CDR-H1 selected from any one of SEQ ID NOs: 2, 23, and 45, the CDR-L3 has at least about 80%, 90%, 91%, 92%, 93%, 94%, 95%, 96%, 97%, 98% or 99% identity to a CDR-L3 selected from any one of SEQ ID NOs: 369 or 395, the CDR-L2 has at least about 80%, 90%, 91%, 92%, 93%, 94%, 95%, 96%, 97%, 98% or 99% identity to a CDR-L2 selected from any one of SEQ ID NOs: 336 or 360, and the CDR-L1 has at least about 80%, 90%, 91%, 92%, 93%, 94%, 95%, 96%, 97%, 98% or 99% identity to a CDR-L1 selected from any one of SEQ ID NOs: 259, 287, and 312. In some embodiments, the CDR-H3 is a CDR-H3 selected from any one of SEQ ID NOs: 176, 204, and 224, with up to 1, 2, 3, 4, 5, 6, 7, or 8 amino acid substitutions; the CDR-H2 is a CDR-H2 selected from any one of SEQ ID NOs: 70, 111, and 142, with up to 1, 2, 3, 4, 5, 6, 7, or 8 amino acid substitutions; the CDR-H1 is a CDR-H1 selected from any one of SEQ ID NOs: 2, 23, and 45, with up to 1, 2, 3, 4, or 5 amino acid substitutions; the CDR-L3 is a CDR-L3 selected from any one of SEQ ID NOs: 369 and 395, with up to 1, 2, 3, 4, or 5 amino acid substitutions; the CDR-L2 is a CDR-L2 selected from any one of SEQ ID NOs: 336 and 360, with up to 1, 2, 3, or 4 amino acid substitutions; and the CDR-L1 is a CDR-L1 selected from any one of SEQ ID NOs: 259, 287, and 312, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 2, 23, and 45; a CDR-H2 comprising an amino acid sequence set forth in any one of SEQ ID NOs: 70, 111, and 142; a CDR-H3 comprising an amino acid sequence set forth in any one of SEQ ID NOs: 176, 204, and 224; a CDR-L1 comprising an amino acid sequence set forth in any one of SEQ ID NOs: 259, 287, and 312; a CDR-L2 comprising an amino acid sequence set forth in any one of SEQ ID NOs: 336 and 360; and a CDR-L3 comprising an amino acid sequence set forth in any one of SEQ ID NOs: 369 and 395.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 177, 205, and 225, the CDR-H2 has at least about 80%, 90%, 91%, 92%, 93%, 94%, 95%, 96%, 97%, 98% or 99% identity to a CDR-H2 selected from any one of SEQ ID NOs: 71, 112, and 143, the CDR-H1 has at least about 80%, 90%, 91%, 92%, 93%, 94%, 95%, 96%, 97%, 98% or 99% identity to a CDR-H1 selected from any one of SEQ ID NOs: 3, 24, and 46, the CDR-L3 has at least about 80%, 90%, 91%, 92%, 93%, 94%, 95%, 96%, 97%, 98% or 99% identity to a CDR-L3 selected from any one of SEQ ID NOs: 370 and 396, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 177, 205, and 225, with up to 1, 2, 3, 4, 5, 6, 7, or 8 amino acid substitutions; the CDR-H2 is a CDR-H2 selected from any one of SEQ ID NOs: 71, 112, and 143, with up to 1, 2, 3, 4, 5, 6, 7, or 8 amino acid substitutions; the CDR-H1 is a CDR-H1 selected from any one of SEQ ID NOs: 3, 24, and 46, with up to 1, 2, 3, 4, or 5 amino acid substitutions; the CDR-L3 is a CDR-L3 selected from any one of SEQ ID NOs: 370 and 396, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequences set forth in any one of SEQ ID NOs: 3, 24, and 46; a CDR-H2 comprising an amino acid sequence set forth in any one of SEQ ID NOs: 71, 112, and 143; a CDR-H3 comprising an amino acid sequence set forth in any one of SEQ ID NOs: 177, 205, and 225; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 370 and 396.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 178, 206, and 226, the CDR-H2 has at least about 80%, 90%, 91%, 92%, 93%, 94%, 95%, 96%, 97%, 98% or 99% identity to a CDR-H2 selected from any one of SEQ ID NOs: 72, 113, and 144, the CDR-H1 has at least about 80%, 90%, 91%, 92%, 93%, 94%, 95%, 96%, 97%, 98% or 99% identity to a CDR-H1 selected from any one of SEQ ID NOs: 4, 22, and 47, the CDR-L3 has at least about 80%, 90%, 91%, 92%, 93%, 94%, 95%, 96%, 97%, 98% or 99% identity to a CDR-L3 selected from any one of SEQ ID NOs: 371 and 397, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 261, 289, and 314. In some embodiments, the CDR-H3 is a CDR-H3 selected from any one of SEQ ID NOs: 178, 206, and 226, with up to 1, 2, 3, 4, 5, 6, 7, or 8 amino acid substitutions; the CDR-H2 is a CDR-H2 selected from any one of SEQ ID NOs: 72, 113, and 144, with up to 1, 2, 3, 4, 5, 6, 7, or 8 amino acid substitutions; the CDR-H1 is a CDR-H1 selected from any one of SEQ ID NOs: 4, 22, and 47, with up to 1, 2, 3, 4, or 5 amino acid substitutions; the CDR-L3 is a CDR-L3 selected from any one of SEQ ID NOs: 371 and 397, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 261, 289, and 314, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 4, 22, and 47; a CDR-H2 comprising an amino acid sequence set forth in any one of SEQ ID NOs: 72, 113, and 144; a CDR-H3 comprising an amino acid sequence set forth in any one of SEQ ID NOs: 178, 206, and 226; a CDR-L1 comprising an amino acid sequence set forth in any one of SEQ ID NOs: 261, 289, and 314; a CDR-L2 comprising an amino acid sequence set forth in any one of SEQ ID NOs: 337 and 361; and a CDR-L3 comprising an amino acid sequence set forth in any one of SEQ ID NOs: 371 and 397.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 179, 207, and 227, the CDR-H2 has at least about 80%, 90%, 91%, 92%, 93%, 94%, 95%, 96%, 97%, 98% or 99% identity to a CDR-H2 selected from any one of SEQ ID NOs: 73, 114, and 145, the CDR-H1 has at least about 80%, 90%, 91%, 92%, 93%, 94%, 95%, 96%, 97%, 98% or 99% identity to a CDR-H1 selected from any one of SEQ ID NOs: 5, 840, and 48, the CDR-L3 has at least about 80%, 90%, 91%, 92%, 93%, 94%, 95%, 96%, 97%, 98% or 99% identity to a CDR-L3 selected from any one of SEQ ID NOs: 372 and 398, the CDR-L2 has at least about 80%, 90%, 91%, 92%, 93%, 94%, 95%, 96%, 97%, 98% or 99% identity to a CDR-L2 selected from any one of SEQ ID NOs: 338 and 362, and the CDR-L1 has at least about 80%, 90%, 91%, 92%, 93%, 94%, 95%, 96%, 97%, 98% or 99% identity to a CDR-L1 selected from any one of SEQ ID NOs: 262, 290, and 315. In some embodiments, the CDR-H3 is a CDR-H3 selected from any one of SEQ ID NOs: 179, 207, and 227, with up to 1, 2, 3, 4, 5, 6, 7, or 8 amino acid substitutions; the CDR-H2 is a CDR-H2 selected from any one of SEQ ID NOs: 73, 114, and 145, with up to 1, 2, 3, 4, 5, 6, 7 or 8 amino acid substitutions; the CDR-H1 is a CDR-H1 selected from any one of SEQ ID NOs: 5, 840, and 48, with up to 1, 2, 3, 4, or 5 amino acid substitutions; the CDR-L3 is a CDR-L3 selected from any one of SEQ ID NOs: 372 and 398, with up to 1, 2, 3, 4, or 5 amino acid substitutions; the CDR-L2 is a CDR-L2 selected from any one of SEQ ID NOs: 338 and 362, with up to 1, 2, 3, or 4 amino acid substitutions; and the CDR-L1 is a CDR-L1 selected from any one of SEQ ID NOs: 262, 290, and 315, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 73, 114, and 145; a CDR-H3 comprising an amino acid sequence set forth in any one of SEQ ID NOs: 179, 207, and 227; a CDR-L1 comprising an amino acid sequence set forth in any one of SEQ ID NOs: 262, 290, and 315; a CDR-L2 comprising an amino acid sequence set forth in any one of SEQ ID NOs: 338 and 362; and a CDR-L3 comprising an amino acid sequence set forth in any one of SEQ ID NOs: 372 and 398.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 180, 208, and 228, the CDR-H2 has at least about 80%, 90%, 91%, 92%, 93%, 94%, 95%, 96%, 97%, 98% or 99% identity to a CDR-H2 selected from any one of SEQ ID NOs: 74, 115, and 146, the CDR-H1 has at least about 80%, 90%, 91%, 92%, 93%, 94%, 95%, 96%, 97%, 98% or 99% identity to a CDR-H1 selected from any one of SEQ ID NOs: 6, 24, and 49, the CDR-L3 has at least about 80%, 90%, 91%, 92%, 93%, 94%, 95%, 96%, 97%, 98% or 99% identity to a CDR-L3 selected from any one of SEQ ID NOs: 373 and 399, the CDR-L2 has at least about 80%, 90%, 91%, 92%, 93%, 94%, 95%, 96%, 97%, 98% or 99% identity to a CDR-L2 selected from any one of SEQ ID NOs: 339 and 361, and the CDR-L1 has at least about 80%, 90%, 91%, 92%, 93%, 94%, 95%, 96%, 97%, 98% or 99% identity to a CDR-L1 selected from any one of SEQ ID NOs: 263, 291, and 316. In some embodiments, the CDR-H3 is a CDR-H3 selected from any one of SEQ ID NOs: 180, 208, and 228, with up to 1, 2, 3, 4, 5, 6, 7, or 8 amino acid substitutions; the CDR-H2 is a CDR-H2 selected from any one of SEQ ID NOs: 74, 115, and 146, with up to 1, 2, 3, 4, 5, 6, 7, or 8 amino acid substitutions; the CDR-H1 is a CDR-H1 selected from any one of SEQ ID NOs: 6, 24, and 49, with up to 1, 2, 3, 4, or 5 amino acid substitutions; the CDR-L3 is a CDR-L3 selected from any one of SEQ ID NOs: 373 and 399, with up to 1, 2, 3, 4 or 5 amino acid substitutions; the CDR-L2 is a CDR-L2 selected from any one of SEQ ID NOs: 339 and 361, with up to 1, 2, 3, or 4 amino acid substitutions; and the CDR-L1 is a CDR-L1 selected from any one of SEQ ID NOs: 263, 291, and 316, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 6, 24, and 49; a CDR-H2 comprising an amino acid sequence set forth in any one of SEQ ID NOs: 74, 115, and 146; a CDR-H3 comprising an amino acid sequence set forth in any one of SEQ ID NOs: 180, 208, and 228; a CDR-L1 comprising an amino acid sequence set forth in any one of SEQ ID NOs: 263, 291, and 316; a CDR-L2 comprising an amino acid sequence set forth in any one of SEQ ID NOs: 339 and 361; and a CDR-L3 comprising an amino acid sequence set forth in any one of SEQ ID NOs: 373 and 399.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 181, 209, and 229, the CDR-H2 has at least about 80%, 90%, 91%, 92%, 93%, 94%, 95%, 96%, 97%, 98% or 99% identity to a CDR-H2 selected from any one of SEQ ID NOs: 75, 116, and 147, the CDR-H1 has at least about 80%, 90%, 91%, 92%, 93%, 94%, 95%, 96%, 97%, 98% or 99% identity to a CDR-H1 selected from any one of SEQ ID NOs: 7, 25, and 50, the CDR-L3 has at least about 80%, 90%, 91%, 92%, 93%, 94%, 95%, 96%, 97%, 98% or 99% identity to a CDR-L3 selected from any one of SEQ ID NOs: 374 and 400, the CDR-L2 has at least about 80%, 90%, 91%, 92%, 93%, 94%, 95%, 96%, 97%, 98% or 99% identity to a CDR-L2 selected from any one of SEQ ID NOs: 340 and 363, and the CDR-L1 has at least about 80%, 90%, 91%, 92%, 93%, 94%, 95%, 96%, 97%, 98% or 99% identity to a CDR-L1 selected from any one of SEQ ID NOs: 264, 292, and 317. In some embodiments, the CDR-H3 is a CDR-H3 selected from any one of SEQ ID NOs: 181, 209, and 229, with up to 1, 2, 3, 4, 5, 6, 7, or 8 amino acid substitutions; the CDR-H2 is a CDR-H2 selected from any one of SEQ ID NOs: 75, 116, and 147, with up to 1, 2, 3, 4, 5, 6, 7, or 8 amino acid substitutions; the CDR-H1 is a CDR-H1 selected from any one of SEQ ID NOs: 7, 25, and 50, with up to 1, 2, 3, 4, or 5 amino acid substitutions; the CDR-L3 is a CDR-L3 selected from any one of SEQ ID NOs: 374 and 400, with up to 1, 2, 3, 4, or 5 amino acid substitutions; the CDR-L2 is a CDR-L2 selected from any one of SEQ ID NOs: 340 and 363, with up to 1, 2, 3, or 4 amino acid substitutions; and the CDR-L1 is a CDR-L1 selected from any one of SEQ ID NOs: 264, 292, and 317, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 7, 25, and 50; a CDR-H2 comprising an amino acid sequence set forth in any one of SEQ ID NOs: 75, 116, and 147; a CDR-H3 comprising an amino acid sequence set forth in any one of SEQ ID NOs: 181, 209, and 229; a CDR-L1 comprising an amino acid sequence set forth in any one of SEQ ID NOs: 264, 292, and 317; a CDR-L2 comprising an amino acid sequence set forth in any one of SEQ ID NOs: 340 and 363; and a CDR-L3 comprising an amino acid sequence set forth in any one of SEQ ID NOs: 374 and 400.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 182, 210, and 230, the CDR-H2 has at least about 80%, 90%, 91%, 92%, 93%, 94%, 95%, 96%, 97%, 98% or 99% identity to a CDR-H2 selected from any one of SEQ ID NOs: 76, 117, and 148, the CDR-H1 has at least about 80%, 90%, 91%, 92%, 93%, 94%, 95%, 96%, 97%, 98% or 99% identity to a CDR-H1 selected from any one of SEQ ID NOs: 8, 26, and 51, the CDR-L3 has at least about 80%, 90%, 91%, 92%, 93%, 94%, 95%, 96%, 97%, 98% or 99% identity to a CDR-L3 selected from any one of SEQ ID NOs: 375 and 401, the CDR-L2 has at least about 80%, 90%, 91%, 92%, 93%, 94%, 95%, 96%, 97%, 98% or 99% identity to a CDR-L2 selected from any one of SEQ ID NOs: 341 and 361, and the CDR-L1 has at least about 80%, 90%, 91%, 92%, 93%, 94%, 95%, 96%, 97%, 98% or 99% identity to a CDR-L1 selected from any one of SEQ ID NOs: 265, 293, and 318. In some embodiments, the CDR-H3 is a CDR-H3 selected from any one of SEQ ID NOs: 182, 210, and 230, with up to 1, 2, 3, 4, 5, 6, 7, or 8 amino acid substitutions; the CDR-H2 is a CDR-H2 selected from any one of SEQ ID NOs: 76, 117, and 148, with up to 1, 2, 3, 4, 5, 6, 7, or 8 amino acid substitutions; the CDR-H1 is a CDR-H1 selected from any one of SEQ ID NOs: 8, 26, and 51, with up to 1, 2, 3, 4, or 5 amino acid substitutions; the CDR-L3 is a CDR-L3 selected from any one of SEQ ID NOs: 375 and 401, with up to 1, 2, 3, 4, or 5 amino acid substitutions; the CDR-L2 is a CDR-L2 selected from any one of SEQ ID NOs: 341 and 361, with up to 1, 2, 3, or 4 amino acid substitutions; and the CDR-L1 is a CDR-L1 selected from any one of SEQ ID NOs: 265, 293, and 318, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 182, 210, and 230; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 375 and 401.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 183, 211, and 231, the CDR-H2 has at least about 80%, 90%, 91%, 92%, 93%, 94%, 95%, 96%, 97%, 98% or 99% identity to a CDR-H2 selected from any one of SEQ ID NOs: 76, 117, and 148, the CDR-H1 has at least about 80%, 90%, 91%, 92%, 93%, 94%, 95%, 96%, 97%, 98% or 99% identity to a CDR-H1 selected from any one of SEQ ID NOs: 8, 26, and 51, the CDR-L3 has at least about 80%, 90%, 91%, 92%, 93%, 94%, 95%, 96%, 97%, 98% or 99% identity to a CDR-L3 selected from any one of SEQ ID NOs: 376 and 402, the CDR-L2 has at least about 80%, 90%, 91%, 92%, 93%, 94%, 95%, 96%, 97%, 98% or 99% identity to a CDR-L2 selected from any one of SEQ ID NOs: 341 and 361, and the CDR-L1 has at least about 80%, 90%, 91%, 92%, 93%, 94%, 95%, 96%, 97%, 98% or 99% identity to a CDR-L1 selected from any one of SEQ ID NOs: 265, 293, and 318. In some embodiments, the CDR-H3 is a CDR-H3 selected from any one of SEQ ID NOs: 183, 211, and 231, with up to 1, 2, 3, 4, 5, 6, 7, or 8 amino acid substitutions; the CDR-H2 is a CDR-H2 selected from any one of SEQ ID NOs: 76, 117, and 148, with up to 1, 2, 3, 4, 5, 6, 7, or 8 amino acid substitutions; the CDR-H1 is a CDR-H1 selected from any one of SEQ ID NOs: 8, 26, and 51, with up to 1, 2, 3, 4, or 5 amino acid substitutions; the CDR-L3 is a CDR-L3 selected from any one of SEQ ID NOs: 376 and 402, with up to 1, 2, 3, 4, or 5 amino acid substitutions; the CDR-L2 is a CDR-L2 selected from any one of SEQ ID NOs: 341 and 361, with up to 1, 2, 3, or 4 amino acid substitutions; and the CDR-L1 is a CDR-L1 selected from any one of SEQ ID NOs: 265, 293, and 318, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 183, 211, and 231; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 376 and 402.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 183, 211, and 231, the CDR-H2 has at least about 80%, 90%, 91%, 92%, 93%, 94%, 95%, 96%, 97%, 98% or 99% identity to a CDR-H2 selected from any one of SEQ ID NOs: 76, 117, and 148, the CDR-H1 has at least about 80%, 90%, 91%, 92%, 93%, 94%, 95%, 96%, 97%, 98% or 99% identity to a CDR-H1 selected from any one of SEQ ID NOs: 8, 26, and 51, the CDR-L3 has at least about 80%, 90%, 91%, 92%, 93%, 94%, 95%, 96%, 97%, 98% or 99% identity to a CDR-L3 selected from any one of SEQ ID NOs: 377 and 403, the CDR-L2 has at least about 80%, 90%, 91%, 92%, 93%, 94%, 95%, 96%, 97%, 98% or 99% identity to a CDR-L2 selected from any one of SEQ ID NOs: 341 and 361, and the CDR-L1 has at least about 80%, 90%, 91%, 92%, 93%, 94%, 95%, 96%, 97%, 98% or 99% identity to a CDR-L1 selected from any one of SEQ ID NOs: 265, 293, and 318. In some embodiments, the CDR-H3 is a CDR-H3 selected from any one of SEQ ID NOs: 183, 211, or 231, with up to 1, 2, 3, 4, 5, 6, 7, or 8 amino acid substitutions; the CDR-H2 is a CDR-H2 selected from any one of SEQ ID NOs: 76, 117, and 148, with up to 1, 2, 3, 4, 5, 6, 7, or 8 amino acid substitutions; the CDR-H1 is a CDR-H1 selected from any one of SEQ ID NOs: 8, 26, and 51, with up to 1, 2, 3, 4, or 5 amino acid substitutions; the CDR-L3 is a CDR-L3 selected from any one of SEQ ID NOs: 377 and 403, with up to 1, 2, 3, 4, or 5 amino acid substitutions; the CDR-L2 is a CDR-L2 selected from any one of SEQ ID NOs: 341 and 361, with up to 1, 2, 3, or 4 amino acid substitutions; and the CDR-L1 is a CDR-L1 selected from any one of SEQ ID NOs: 265, 293, and 318, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 183, 211, and 231; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 377 and 403.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 184, 212, and 232, the CDR-H2 has at least about 80%, 90%, 91%, 92%, 93%, 94%, 95%, 96%, 97%, 98% or 99% identity to a CDR-H2 selected from any one of SEQ ID NOs: 76, 117, and 148, the CDR-H1 has at least about 80%, 90%, 91%, 92%, 93%, 94%, 95%, 96%, 97%, 98% or 99% identity to a CDR-H1 selected from any one of SEQ ID NOs: 8, 26, and 51, the CDR-L3 has at least about 80%, 90%, 91%, 92%, 93%, 94%, 95%, 96%, 97%, 98% or 99% identity to a CDR-L3 selected from any one of SEQ ID NOs: 376 and 402, the CDR-L2 has at least about 80%, 90%, 91%, 92%, 93%, 94%, 95%, 96%, 97%, 98% or 99% identity to a CDR-L2 selected from any one of SEQ ID NOs: 341 and 361, and the CDR-L1 has at least about 80%, 90%, 91%, 92%, 93%, 94%, 95%, 96%, 97%, 98% or 99% identity to a CDR-L1 selected from any one of SEQ ID NOs: 265, 293, and 318. In some embodiments, the CDR-H3 is a CDR-H3 selected from any one of SEQ ID NOs: 184, 212, and 232, with up to 1, 2, 3, 4, 5, 6, 7, or 8 amino acid substitutions; the CDR-H2 is a CDR-H2 selected from any one of SEQ ID NOs: 76, 117, and 148, with up to 1, 2, 3, 4, 5, 6, 7, or 8 amino acid substitutions; the CDR-H1 is a CDR-H1 selected from any one of SEQ ID NOs: 8, 26, and 51, with up to 1, 2, 3, 4, or 5 amino acid substitutions; the CDR-L3 is a CDR-L3 selected from any one of SEQ ID NOs: 376 and 402, with up to 1, 2, 3, 4, or 5 amino acid substitutions; the CDR-L2 is a CDR-L2 selected from any one of SEQ ID NOs: 341 and 361, with up to 1, 2, 3, or 4 amino acid substitutions; and the CDR-L1 is a CDR-L1 selected from any one of SEQ ID NOs: 265, 293, and 318, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 184, 212, and 232; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 376 and 402.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 184, 212, and 232, the CDR-H2 has at least about 80%, 90%, 91%, 92%, 93%, 94%, 95%, 96%, 97%, 98% or 99% identity to a CDR-H2 selected from any one of SEQ ID NOs: 76, 117, and 148, the CDR-H1 has at least about 80%, 90%, 91%, 92%, 93%, 94%, 95%, 96%, 97%, 98% or 99% identity to a CDR-H1 selected from any one of SEQ ID NOs: 8, 26, and 51, the CDR-L3 has at least about 80%, 90%, 91%, 92%, 93%, 94%, 95%, 96%, 97%, 98% or 99% identity to a CDR-L3 selected from any one of SEQ ID NOs: 377 and 403, the CDR-L2 has at least about 80%, 90%, 91%, 92%, 93%, 94%, 95%, 96%, 97%, 98% or 99% identity to a CDR-L2 selected from any one of SEQ ID NOs: 341 or 361, and the CDR-L1 has at least about 80%, 90%, 91%, 92%, 93%, 94%, 95%, 96%, 97%, 98% or 99% identity to a CDR-L1 selected from any one of SEQ ID NOs: 265, 293, and 318. In some embodiments, the CDR-H3 is a CDR-H3 selected from any one of SEQ ID NOs: 184, 212, and 232, with up to 1, 2, 3, 4, 5, 6, 7, or 8 amino acid substitutions; the CDR-H2 is a CDR-H2 selected from any one of SEQ ID NOs: 76, 117, and 148, with up to 1, 2, 3, 4, 5, 6, 7, or 8 amino acid substitutions; the CDR-H1 is a CDR-H1 selected from any one of SEQ ID NOs: 8, 26, and 51, with up to 1, 2, 3, 4, or 5 amino acid substitutions; the CDR-L3 is a CDR-L3 selected from any one of SEQ ID NOs: 377 and 403, with up to 1, 2, 3, 4, or 5 amino acid substitutions; the CDR-L2 is a CDR-L2 selected from any one of SEQ ID NOs: 341 and 361, with up to 1, 2, 3, or 4 amino acid substitutions; and the CDR-L1 is a CDR-L1 selected from any one of SEQ ID NOs: 265, 293, and 318, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 184, 212, and 232; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 377 and 403.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 185, 213, and 233, the CDR-H2 has at least about 80%, 90%, 91%, 92%, 93%, 94%, 95%, 96%, 97%, 98% or 99% identity to a CDR-H2 selected from any one of SEQ ID NOs: 76, 117, and 148, the CDR-H1 has at least about 80%, 90%, 91%, 92%, 93%, 94%, 95%, 96%, 97%, 98% or 99% identity to a CDR-H1 selected from any one of SEQ ID NOs: 8, 26, and 51, the CDR-L3 has at least about 80%, 90%, 91%, 92%, 93%, 94%, 95%, 96%, 97%, 98% or 99% identity to a CDR-L3 selected from any one of SEQ ID NOs: 376 and 402, the CDR-L2 has at least about 80%, 90%, 91%, 92%, 93%, 94%, 95%, 96%, 97%, 98% or 99% identity to a CDR-L2 selected from any one of SEQ ID NOs: 341 or 361, and the CDR-L1 has at least about 80%, 90%, 91%, 92%, 93%, 94%, 95%, 96%, 97%, 98% or 99% identity to a CDR-L1 selected from any one of SEQ ID NOs: 265, 293, and 318. In some embodiments, the CDR-H3 is a CDR-H3 selected from any one of SEQ ID NOs: 185, 213, and 233, with up to 1, 2, 3, 4, 5, 6, 7, or 8 amino acid substitutions; the CDR-H2 is a CDR-H2 selected from any one of SEQ ID NOs: 76, 117, and 148, with up to 1, 2, 3, 4, 5, 6, 7, or 8 amino acid substitutions; the CDR-H1 is a CDR-H1 selected from any one of SEQ ID NOs: 8, 26, and 51, with up to 1, 2, 3, 4, or 5 amino acid substitutions; the CDR-L3 is a CDR-L3 selected from any one of SEQ ID NOs: 376 and 402, with up to 1, 2, 3, 4, or 5 amino acid substitutions; the CDR-L2 is a CDR-L2 selected from any one of SEQ ID NOs: 341 and 361, with up to 1, 2, 3, or 4 amino acid substitutions; and the CDR-L1 is a CDR-L1 selected from any one of SEQ ID NOs: 265, 293, and 318, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 185, 213, and 233; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 376 and 402.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 185, 213, and 233, the CDR-H2 has at least about 80%, 90%, 91%, 92%, 93%, 94%, 95%, 96%, 97%, 98% or 99% identity to a CDR-H2 selected from any one of SEQ ID NOs: 76, 117, and 148, the CDR-H1 has at least about 80%, 90%, 91%, 92%, 93%, 94%, 95%, 96%, 97%, 98% or 99% identity to a CDR-H1 selected from any one of SEQ ID NOs: 8, 26, and 51, the CDR-L3 has at least about 80%, 90%, 91%, 92%, 93%, 94%, 95%, 96%, 97%, 98% or 99% identity to a CDR-L3 selected from any one of SEQ ID NOs: 377 and 403, the CDR-L2 has at least about 80%, 90%, 91%, 92%, 93%, 94%, 95%, 96%, 97%, 98% or 99% identity to a CDR-L2 selected from any one of SEQ ID NOs: 341 or 361, and the CDR-L1 has at least about 80%, 90%, 91%, 92%, 93%, 94%, 95%, 96%, 97%, 98% or 99% identity to a CDR-L1 selected from any one of SEQ ID NOs: 265, 293, and 318. In some embodiments, the CDR-H3 is a CDR-H3 selected from any one of SEQ ID NOs: 185, 213, and 233, with up to 1, 2, 3, 4, 5, 6, 7, or 8 amino acid substitutions; the CDR-H2 is a CDR-H2 selected from any one of SEQ ID NOs: 76, 117, and 148, with up to 1, 2, 3, 4, 5, 6, 7, or 8 amino acid substitutions; the CDR-H1 is a CDR-H1 selected from any one of SEQ ID NOs: 8, 26, and 51, with up to 1, 2, 3, 4, or 5 amino acid substitutions; the CDR-L3 is a CDR-L3 selected from any one of SEQ ID NOs: 377 and 403, with up to 1, 2, 3, 4, or 5 amino acid substitutions; the CDR-L2 is a CDR-L2 selected from any one of SEQ ID NOs: 341 and 361, with up to 1, 2, 3, or 4 amino acid substitutions; and the CDR-L1 is a CDR-L1 selected from any one of SEQ ID NOs: 265, 293, and 318, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 76, 117, and 148; a CDR-H3 comprising an amino acid sequence set forth in any one of SEQ ID NOs: 185, 213, and 233; a CDR-L1 comprising an amino acid sequence set forth in any one of SEQ ID NOs: 265, 293, and 318; a CDR-L2 comprising an amino acid sequence set forth in any one of SEQ ID NOs: 341 and 361; and a CDR-L3 comprising an amino acid sequence set forth in any one of SEQ ID NOs: 377 and 403.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175, 203, and 234, the CDR-H2 has at least about 80%, 90%, 91%, 92%, 93%, 94%, 95%, 96%, 97%, 98% or 99% identity to a CDR-H2 selected from any one of SEQ ID NOs: 77, 118, and 149, the CDR-H1 has at least about 80%, 90%, 91%, 92%, 93%, 94%, 95%, 96%, 97%, 98% or 99% identity to a CDR-H1 selected from any one of SEQ ID NOs: 9, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 368 and 394, the CDR-L2 has at least about 80%, 90%, 91%, 92%, 93%, 94%, 95%, 96%, 97%, 98% or 99% identity to a CDR-L2 selected from any one of SEQ ID NOs: 342 and 364, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 175, 203, and 234, with up to 1, 2, 3, 4, 5, 6, 7, or 8 amino acid substitutions; the CDR-H2 is a CDR-H2 selected from any one of SEQ ID NOs: 77, 118, and 149, with up to 1, 2, 3, 4, 5, 6, 7, or 8 amino acid substitutions; the CDR-H1 is a CDR-H1 selected from any one of SEQ ID NOs: 9, 27, and 52, with up to 1, 2, 3, 4, or 5 amino acid substitutions; the CDR-L3 is a CDR-L3 selected from any one of SEQ ID NOs: 368 and 394, with up to 1, 2, 3, 4, or 5 amino acid substitutions; the CDR-L2 is a CDR-L2 selected from any one of SEQ ID NOs: 342 and 364, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 9, 27, and 52; a CDR-H2 comprising an amino acid sequence set forth in any one of SEQ ID NOs: 77, 118, and 149; a CDR-H3 comprising an amino acid sequence set forth in any one of SEQ ID NOs: 175, 203, and 234; a CDR-L1 comprising an amino acid sequence set forth in any one of SEQ ID NOs: 260, 288, and 313; a CDR-L2 comprising an amino acid sequence set forth in any one of SEQ ID NOs: 342 and 364; and a CDR-L3 comprising an amino acid sequence set forth in any one of SEQ ID NOs: 368 and 394.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175, 203, and 223, the CDR-H2 has at least about 80%, 90%, 91%, 92%, 93%, 94%, 95%, 96%, 97%, 98% or 99% identity to a CDR-H2 selected from any one of SEQ ID NOs: 78, 119, and 150, the CDR-H1 has at least about 80%, 90%, 91%, 92%, 93%, 94%, 95%, 96%, 97%, 98% or 99% identity to a CDR-H1 selected from any one of SEQ ID NOs: 1, 28, and 53, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 343 and 365, and the CDR-L1 has at least about 80%, 90%, 91%, 92%, 93%, 94%, 95%, 96%, 97%, 98% or 99% identity to a CDR-L1 selected from any one of SEQ ID NOs: 266, 286, and 311. In some embodiments, the CDR-H3 is a CDR-H3 selected from any one of SEQ ID NOs: 175, 203, and 223, with up to 1, 2, 3, 4, 5, 6, 7, or 8 amino acid substitutions; the CDR-H2 is a CDR-H2 selected from any one of SEQ ID NOs: 78, 119, and 150, with up to 1, 2, 3, 4, 5, 6, 7, or 8 amino acid substitutions; the CDR-H1 is a CDR-H1 selected from any one of SEQ ID NOs: 1, 28, and 53, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 343 and 365, with up to 1, 2, 3, or 4 amino acid substitutions; and the CDR-L1 is a CDR-L1 selected from any one of SEQ ID NOs: 266, 286, and 311, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 1, 28, and 53; a CDR-H2 comprising an amino acid sequence set forth in any one of SEQ ID NOs: 78, 119, and 150; a CDR-H3 comprising an amino acid sequence set forth in any one of SEQ ID NOs: 175, 203, and 223; a CDR-L1 comprising an amino acid sequence set forth in any one of SEQ ID NOs: 266, 286, and 311; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 186, 203, and 235, the CDR-H2 has at least about 80%, 90%, 91%, 92%, 93%, 94%, 95%, 96%, 97%, 98% or 99% identity to a CDR-H2 selected from any one of SEQ ID NOs: 79, 119, and 150, the CDR-H1 has at least about 80%, 90%, 91%, 92%, 93%, 94%, 95%, 96%, 97%, 98% or 99% identity to a CDR-H1 selected from any one of SEQ ID NOs: 10, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 344 and 365, and the CDR-L1 has at least about 80%, 90%, 91%, 92%, 93%, 94%, 95%, 96%, 97%, 98% or 99% identity to a CDR-L1 selected from any one of SEQ ID NOs: 267, 294, and 319. In some embodiments, the CDR-H3 is a CDR-H3 selected from any one of SEQ ID NOs: 186, 203, and 235, with up to 1, 2, 3, 4, 5, 6, 7, or 8 amino acid substitutions; the CDR-H2 is a CDR-H2 selected from any one of SEQ ID NOs: 79, 119, and 150, with up to 1, 2, 3, 4, 5, 6, 7, or 8 amino acid substitutions; the CDR-H1 is a CDR-H1 selected from any one of SEQ ID NOs: 10, 27, and 52, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 344 and 365, with up to 1, 2, 3, or 4 amino acid substitutions; and the CDR-L1 is a CDR-L1 selected from any one of SEQ ID NOs: 267, 294, and 319, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 79, 119, and 150; a CDR-H3 comprising an amino acid sequence set forth in any one of SEQ ID NOs: 186, 203, and 235; a CDR-L1 comprising an amino acid sequence set forth in any one of SEQ ID NOs: 267, 294, and 319; a CDR-L2 comprising an amino acid sequence set forth in any one of SEQ ID NOs: 344 and 365; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 186, 203, and 235, the CDR-H2 has at least about 80%, 90%, 91%, 92%, 93%, 94%, 95%, 96%, 97%, 98% or 99% identity to a CDR-H2 selected from any one of SEQ ID NOs: 80, 120, and 151, the CDR-H1 has at least about 80%, 90%, 91%, 92%, 93%, 94%, 95%, 96%, 97%, 98% or 99% identity to a CDR-H1 selected from any one of SEQ ID NOs: 10, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 343 and 365, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 186, 203, and 235, with up to 1, 2, 3, 4, 5, 6, 7, or 8 amino acid substitutions; the CDR-H2 is a CDR-H2 selected from any one of SEQ ID NOs: 80, 120, and 151, with up to 1, 2, 3, 4, 5, 6, 7, or 8 amino acid substitutions; the CDR-H1 is a CDR-H1 selected from any one of SEQ ID NOs: 10, 27, and 52, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 343 and 365, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 80, 120, and 151; a CDR-H3 comprising an amino acid sequence set forth in any one of SEQ ID NOs: 186, 203, and 235; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175, 203, and 223, the CDR-H2 has at least about 80%, 90%, 91%, 92%, 93%, 94%, 95%, 96%, 97%, 98% or 99% identity to a CDR-H2 selected from any one of SEQ ID NOs: 79, 119, and 150, the CDR-H1 has at least about 80%, 90%, 91%, 92%, 93%, 94%, 95%, 96%, 97%, 98% or 99% identity to a CDR-H1 selected from any one of SEQ ID NOs: 11, 27, or 52, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 343 and 365, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 175, 203, and 223, with up to 1, 2, 3, 4, 5, 6, 7, or 8 amino acid substitutions; the CDR-H2 is a CDR-H2 selected from any one of SEQ ID NOs: 79, 119, and 150, with up to 1, 2, 3, 4, 5, 6, 7, or 8 amino acid substitutions; the CDR-H1 is a CDR-H1 selected from any one of SEQ ID NOs: 11, 27, and 52, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 343 and 365, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 11, 27, and 52; a CDR-H2 comprising an amino acid sequence set forth in any one of SEQ ID NOs: 79, 119, and 150; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175, 203, and 223, the CDR-H2 has at least about 80%, 90%, 91%, 92%, 93%, 94%, 95%, 96%, 97%, 98% or 99% identity to a CDR-H2 selected from any one of SEQ ID NOs: 81, 121, and 152, the CDR-H1 has at least about 80%, 90%, 91%, 92%, 93%, 94%, 95%, 96%, 97%, 98% or 99% identity to a CDR-H1 selected from any one of SEQ ID NOs: 11, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 343 and 365, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 175, 203, and 223, with up to 1, 2, 3, 4, 5, 6, 7, or 8 amino acid substitutions; the CDR-H2 is a CDR-H2 selected from any one of SEQ ID NOs: 81, 121, and 152, with up to 1, 2, 3, 4, 5, 6, 7, or 8 amino acid substitutions; the CDR-H1 is a CDR-H1 selected from any one of SEQ ID NOs: 11, 27, and 52, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 343 and 365, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 11, 27, and 52; a CDR-H2 comprising an amino acid sequence set forth in any one of SEQ ID NOs: 81, 121, and 152; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 187, 203, and 236, the CDR-H2 has at least about 80%, 90%, 91%, 92%, 93%, 94%, 95%, 96%, 97%, 98% or 99% identity to a CDR-H2 selected from any one of SEQ ID NOs: 80, 120, and 151, the CDR-H1 has at least about 80%, 90%, 91%, 92%, 93%, 94%, 95%, 96%, 97%, 98% or 99% identity to a CDR-H1 selected from any one of SEQ ID NOs: 10, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 343 and 365, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 187, 203, and 236, with up to 1, 2, 3, 4, 5, 6, 7, or 8 amino acid substitutions; the CDR-H2 is a CDR-H2 selected from any one of SEQ ID NOs: 80, 120, and 151, with up to 1, 2, 3, 4, 5, 6, 7, or 8 amino acid substitutions; the CDR-H1 is a CDR-H1 selected from any one of SEQ ID NOs: 10, 27, and 52, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 343 and 365, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 80, 120, and 151; a CDR-H3 comprising an amino acid sequence set forth in any one of SEQ ID NOs: 187, 203, and 236; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 187, 203, and 236, the CDR-H2 has at least about 80%, 90%, 91%, 92%, 93%, 94%, 95%, 96%, 97%, 98% or 99% identity to a CDR-H2 selected from any one of SEQ ID NOs: 79, 119, and 150, the CDR-H1 has at least about 80%, 90%, 91%, 92%, 93%, 94%, 95%, 96%, 97%, 98% or 99% identity to a CDR-H1 selected from any one of SEQ ID NOs: 10, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 343 and 365, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 187, 203, and 236, with up to 1, 2, 3, 4, 5, 6, 7, or 8 amino acid substitutions; the CDR-H2 is a CDR-H2 selected from any one of SEQ ID NOs: 79, 119, and 150, with up to 1, 2, 3, 4, 5, 6, 7, or 8 amino acid substitutions; the CDR-H1 is a CDR-H1 selected from any one of SEQ ID NOs: 10, 27, and 52, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 343 and 365, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 79, 119, and 150; a CDR-H3 comprising an amino acid sequence set forth in any one of SEQ ID NOs: 187, 203, and 236; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 188, 203, and 237, the CDR-H2 has at least about 80%, 90%, 91%, 92%, 93%, 94%, 95%, 96%, 97%, 98% or 99% identity to a CDR-H2 selected from any one of SEQ ID NOs: 70, 111, and 142, the CDR-H1 has at least about 80%, 90%, 91%, 92%, 93%, 94%, 95%, 96%, 97%, 98% or 99% identity to a CDR-H1 selected from any one of SEQ ID NOs: 10, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 368 and 394, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from SEQ ID NOs: 268, 288, and 313. In some embodiments, the CDR-H3 is a CDR-H3 any one of selected from SEQ ID NOs: 188, 203, and 237, with up to 1, 2, 3, 4, 5, 6, 7, or 8 amino acid substitutions; the CDR-H2 is a CDR-H2 selected from any one of SEQ ID NOs: 70, 111, and 142, with up to 1, 2, 3, 4, 5, 6, 7, or 8 amino acid substitutions; the CDR-H1 is a CDR-H1 selected from any one of SEQ ID NOs: 10, 27, and 52, with up to 1, 2, 3, 4, or 5 amino acid substitutions; the CDR-L3 is a CDR-L3 selected from any one of SEQ ID NOs: 368 and 394, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 268, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 188, 203, and 237; a CDR-L1 comprising an amino acid sequence set forth in any one of SEQ ID NOs: 268, 288, and 313; a CDR-L2 comprising an amino acid sequence set forth in any one of SEQ ID NOs: 337 and 361; and a CDR-L3 comprising an amino acid sequence set forth in any one of SEQ ID NOs: 368 and 394.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 188, 203, and 237, the CDR-H2 has at least about 80%, 90%, 91%, 92%, 93%, 94%, 95%, 96%, 97%, 98% or 99% identity to a CDR-H2 selected from any one of SEQ ID NOs: 70, 111, and 142, the CDR-H1 has at least about 80%, 90%, 91%, 92%, 93%, 94%, 95%, 96%, 97%, 98% or 99% identity to a CDR-H1 selected from any one of SEQ ID NOs: 12, 29, and 54, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 343 and 365, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 188, 203, and 237, with up to 1, 2, 3, 4, 5, 6, 7, or 8 amino acid substitutions; the CDR-H2 is a CDR-H2 selected from any one of SEQ ID NOs: 70, 111, and 142, with up to 1, 2, 3, 4, 5, 6, 7, or 8 amino acid substitutions; the CDR-H1 is a CDR-H1 selected from any one of SEQ ID NOs: 12, 29, and 54, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 343 and 365, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 12, 29, and 54; a CDR-H2 comprising an amino acid sequence set forth in any one of SEQ ID NOs: 70, 111, and 142; a CDR-H3 comprising an amino acid sequence set forth in any one of SEQ ID NOs: 188, 203, and 237; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 189, 214, and 238, the CDR-H2 has at least about 80%, 90%, 91%, 92%, 93%, 94%, 95%, 96%, 97%, 98% or 99% identity to a CDR-H2 selected from any one of SEQ ID NOs: 82, 111, and 142, the CDR-H1 has at least about 80%, 90%, 91%, 92%, 93%, 94%, 95%, 96%, 97%, 98% or 99% identity to a CDR-H1 selected from any one of SEQ ID NOs: 10, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 345 and 361, and the CDR-L1 has at least about 80%, 90%, 91%, 92%, 93%, 94%, 95%, 96%, 97%, 98% or 99% identity to a CDR-L1 selected from any one of SEQ ID NOs: 269, 288, and 313. In some embodiments, the CDR-H3 is a CDR-H3 selected from any one of SEQ ID NOs: 189, 214, and 238, with up to 1, 2, 3, 4, 5, 6, 7, or 8 amino acid substitutions; the CDR-H2 is a CDR-H2 selected from any one of SEQ ID NOs: 82, 111, and 142, with up to 1, 2, 3, 4, 5, 6, 7, or 8 amino acid substitutions; the CDR-H1 is a CDR-H1 selected from SEQ ID NO: 10, 27, and 52, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 345 and 361, with up to 1, 2, 3, or 4 amino acid substitutions; and the CDR-L1 is a CDR-L1 selected from any one of SEQ ID NOs: 269, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 82, 111, and 142; a CDR-H3 comprising an amino acid sequence set forth in any one of SEQ ID NOs: 189, 214, and 238; a CDR-L1 comprising an amino acid sequence set forth in any one of SEQ ID NOs: 269, 288, and 313; a CDR-L2 comprising an amino acid sequence set forth in any one of SEQ ID NOs: 345 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175, 203, and 234, the CDR-H2 has at least about 80%, 90%, 91%, 92%, 93%, 94%, 95%, 96%, 97%, 98% or 99% identity to a CDR-H2 selected from any one of SEQ ID NOs: 83, 122, and 153, the CDR-H1 has at least about 80%, 90%, 91%, 92%, 93%, 94%, 95%, 96%, 97%, 98% or 99% identity to a CDR-H1 selected from any one of SEQ ID NOs: 10, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 346 or 361, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 175, 203, and 234, with up to 1, 2, 3, 4, 5, 6, 7, or 8 amino acid substitutions; the CDR-H2 is a CDR-H2 selected from any one of SEQ ID NOs: 83, 122, and 153, with up to 1, 2, 3, 4, 5, 6, 7, or 8 amino acid substitutions; the CDR-H1 is a CDR-H1 selected from SEQ ID NO: 10, 27, and 52, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 346 and 361, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 83, 122, and 153; a CDR-H3 comprising an amino acid sequence set forth in any one of SEQ ID NOs: 175, 203, and 234; a CDR-L1 comprising an amino acid sequence set forth in any one of SEQ ID NOs: 260, 288, and 313; a CDR-L2 comprising an amino acid sequence set forth in any one of SEQ ID NOs: 346 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 188, 203, and 239, the CDR-H2 has at least about 80%, 90%, 91%, 92%, 93%, 94%, 95%, 96%, 97%, 98% or 99% identity to a CDR-H2 selected from any one of SEQ ID NOs: 84, 111, and 142, the CDR-H1 has at least about 80%, 90%, 91%, 92%, 93%, 94%, 95%, 96%, 97%, 98% or 99% identity to a CDR-H1 selected from any one of SEQ ID NOs: 13, 30, and 55, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 337 or 361, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 188, 203, and 239, with up to 1, 2, 3, 4, 5, 6, 7, or 8 amino acid substitutions; the CDR-H2 is a CDR-H2 selected from any one of SEQ ID NOs: 84, 111, and 142, with up to 1, 2, 3, 4, 5, 6, 7, or 8 amino acid substitutions; the CDR-H1 is a CDR-H1 selected from SEQ ID NO: 13, 30, and 55, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 13, 30, and 55; a CDR-H2 comprising an amino acid sequence set forth in any one of SEQ ID NOs: 84, 111, and 142; a CDR-H3 comprising an amino acid sequence set forth in any one of SEQ ID NOs: 188, 203, and 239; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175, 203, and 234, the CDR-H2 has at least about 80%, 90%, 91%, 92%, 93%, 94%, 95%, 96%, 97%, 98% or 99% identity to a CDR-H2 selected from any one of SEQ ID NOs: 70, 111, and 142, the CDR-H1 has at least about 80%, 90%, 91%, 92%, 93%, 94%, 95%, 96%, 97%, 98% or 99% identity to a CDR-H1 selected from any one of SEQ ID NOs: 10, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 347 and 365, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 175, 203, and 234, with up to 1, 2, 3, 4, 5, 6, 7, or 8 amino acid substitutions; the CDR-H2 is a CDR-H2 selected from any one of SEQ ID NOs: 70, 111, and 142, with up to 1, 2, 3, 4, 5, 6, 7, or 8 amino acid substitutions; the CDR-H1 is a CDR-H1 selected from any one of SEQ ID NOs: 10, 27, and 52, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 347 and 365, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 234; a CDR-L1 comprising an amino acid sequence set forth in any one of SEQ ID NOs: 260, 288, and 313; a CDR-L2 comprising an amino acid sequence set forth in any one of SEQ ID NOs: 347 and 365; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175, 203, and 234, the CDR-H2 has at least about 80%, 90%, 91%, 92%, 93%, 94%, 95%, 96%, 97%, 98% or 99% identity to a CDR-H2 selected from any one of SEQ ID NOs: 70, 111, and 142, the CDR-H1 has at least about 80%, 90%, 91%, 92%, 93%, 94%, 95%, 96%, 97%, 98% or 99% identity to a CDR-H1 selected from any one of SEQ ID NOs: 10, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, CDR-L2 has at least about 80%, 90%, 91%, 92%, 93%, 94%, 95%, 96%, 97%, 98% or 99% identity to a CDR-L2 selected from any one of SEQ ID NOs: 345 or 361, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 175, 203, and 234, with up to 1, 2, 3, 4, 5, 6, 7, or 8 amino acid substitutions; the CDR-H2 is a CDR-H2 selected from any one of SEQ ID NOs: 70, 111, and 142, with up to 1, 2, 3, 4, 5, 6, 7, or 8 amino acid substitutions; the CDR-H1 is a CDR-H1 selected from any one of SEQ ID NOs: 10, 27, and 52, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 345 and 361, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 234; a CDR-L1 comprising an amino acid sequence set forth in any one of SEQ ID NOs: 260, 288, and 313; a CDR-L2 comprising an amino acid sequence set forth in any one of SEQ ID NOs: 345 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 188, 203, and 239, the CDR-H2 has at least about 80%, 90%, 91%, 92%, 93%, 94%, 95%, 96%, 97%, 98% or 99% identity to a CDR-H2 selected from any one of SEQ ID NOs: 85, 123, and 154, the CDR-H1 has at least about 80%, 90%, 91%, 92%, 93%, 94%, 95%, 96%, 97%, 98% or 99% identity to a CDR-H1 selected from any one of SEQ ID NOs: 10, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 337 or 361, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 188, 203, and 239, with up to 1, 2, 3, 4, 5, 6, 7, or 8 amino acid substitutions; the CDR-H2 is a CDR-H2 selected from any one of SEQ ID NOs: 85, 123, and 154, with up to 1, 2, 3, 4, 5, 6, 7, or 8 amino acid substitutions; the CDR-H1 is a CDR-H1 selected from any one of SEQ ID NOs: 10, 27, and 52, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 85, 123, and 154; a CDR-H3 comprising an amino acid sequence set forth in any one of SEQ ID NOs: 188, 203, and 239; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175, 203, and 223, the CDR-H2 has at least about 80%, 90%, 91%, 92%, 93%, 94%, 95%, 96%, 97%, 98% or 99% identity to a CDR-H2 selected from any one of SEQ ID NOs: 70, 111, and 142, the CDR-H1 has at least about 80%, 90%, 91%, 92%, 93%, 94%, 95%, 96%, 97%, 98% or 99% identity to a CDR-H1 selected from any one of SEQ ID NOs: 10, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 270, 295, and 313. In some embodiments, the CDR-H3 is a CDR-H3 selected from any one of SEQ ID NOs: 175, 203, and 223, with up to 1, 2, 3, 4, 5, 6, 7, or 8 amino acid substitutions; the CDR-H2 is a CDR-H2 selected from any one of SEQ ID NOs: 70, 111, and 142, with up to 1, 2, 3, 4, 5, 6, 7, or 8 amino acid substitutions; the CDR-H1 is a CDR-H1 selected from any one of SEQ ID NOs: 10, 27, and 52, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 270, 295, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 270, 295, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175, 203, or 223, the CDR-H2 has at least about 80%, 90%, 91%, 92%, 93%, 94%, 95%, 96%, 97%, 98% or 99% identity to a CDR-H2 selected from any one of SEQ ID NOs: 70, 111, and 142, the CDR-H1 has at least about 80%, 90%, 91%, 92%, 93%, 94%, 95%, 96%, 97%, 98% or 99% identity to a CDR-H1 selected from any one of SEQ ID NOs: 1, 31, and 56, the CDR-L3 has at least about 80%, 90%, 91%, 92%, 93%, 94%, 95%, 96%, 97%, 98% or 99% identity to a CDR-L3 selected from any one of SEQ ID NOs: 379 and 405, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 175, 203, and 223, with up to 1, 2, 3, 4, 5, 6, 7, or 8 amino acid substitutions; the CDR-H2 is a CDR-H2 selected from any one of SEQ ID NOs: 70, 111, and 142, with up to 1, 2, 3, 4, 5, 6, 7, or 8 amino acid substitutions; the CDR-H1 is a CDR-H1 selected from any one of SEQ ID NOs: 1, 31, and 56, with up to 1, 2, 3, 4, or 5 amino acid substitutions; the CDR-L3 is a CDR-L3 selected from any one of SEQ ID NOs: 379 and 405, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 1, 31, and 56; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 379 and 405.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175, 203, and 223, the CDR-H2 has at least about 80%, 90%, 91%, 92%, 93%, 94%, 95%, 96%, 97%, 98% or 99% identity to a CDR-H2 selected from any one of SEQ ID NOs: 86, 124, and 155, the CDR-H1 has at least about 80%, 90%, 91%, 92%, 93%, 94%, 95%, 96%, 97%, 98% or 99% identity to a CDR-H1 selected from any one of SEQ ID NOs: 1, 32, and 57, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 175, 203, and 223, with up to 1, 2, 3, 4, 5, 6, 7, or 8 amino acid substitutions; the CDR-H2 is a CDR-H2 selected from any one of SEQ ID NOs: 86, 124, and 155, with up to 1, 2, 3, 4, 5, 6, 7, or 8 amino acid substitutions; the CDR-H1 is a CDR-H1 selected from any one of SEQ ID NOs: 1, 32, and 57, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 1, 32, and 57; a CDR-H2 comprising an amino acid sequence set forth in any one of SEQ ID NOs: 86, 124, and 155; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175, 203, and 223, the CDR-H2 has at least about 80%, 90%, 91%, 92%, 93%, 94%, 95%, 96%, 97%, 98% or 99% identity to a CDR-H2 selected from any one of SEQ ID NOs: 70, 111, and 142, the CDR-H1 has at least about 80%, 90%, 91%, 92%, 93%, 94%, 95%, 96%, 97%, 98% or 99% identity to a CDR-H1 selected from any one of SEQ ID NOs: 10, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 380 and 406, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 175, 203, and 223, with up to 1, 2, 3, 4, 5, 6, 7, or 8 amino acid substitutions; the CDR-H2 is a CDR-H2 selected from any one of SEQ ID NOs: 70, 111, and 142, with up to 1, 2, 3, 4, 5, 6, 7, or 8 amino acid substitutions; the CDR-H1 is a CDR-H1 selected from any one of SEQ ID NOs: 10, 27, and 52, with up to 1, 2, 3, 4, or 5 amino acid substitutions; the CDR-L3 is a CDR-L3 selected from any one of SEQ ID NOs: 380 and 406, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 380 and 406.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175, 203, and 223, the CDR-H2 has at least about 80%, 90%, 91%, 92%, 93%, 94%, 95%, 96%, 97%, 98% or 99% identity to a CDR-H2 selected from any one of SEQ ID NOs: 70, 111, and 142, the CDR-H1 has at least about 80%, 90%, 91%, 92%, 93%, 94%, 95%, 96%, 97%, 98% or 99% identity to a CDR-H1 selected from any one of SEQ ID NOs: 14, 33, and 58, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 175, 203, and 223, with up to 1, 2, 3, 4, 5, 6, 7, or 8 amino acid substitutions; the CDR-H2 is a CDR-H2 selected from any one of SEQ ID NOs: 70, 111, and 142, with up to 1, 2, 3, 4, 5, 6, 7, or 8 amino acid substitutions; the CDR-H1 is a CDR-H1 selected from any one of SEQ ID NOs: 14, 33, and 58, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 14, 33, and 58; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175, 203, and 223, the CDR-H2 has at least about 80%, 90%, 91%, 92%, 93%, 94%, 95%, 96%, 97%, 98% or 99% identity to a CDR-H2 selected from any one of SEQ ID NOs: 87, 111, and 142, the CDR-H1 has at least about 80%, 90%, 91%, 92%, 93%, 94%, 95%, 96%, 97%, 98% or 99% identity to a CDR-H1 selected from any one of SEQ ID NOs: 10, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 343 and 365, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 175, 203, and 223, with up to 1, 2, 3, 4, 5, 6, 7, or 8 amino acid substitutions; the CDR-H2 is a CDR-H2 selected from any one of SEQ ID NOs: 87, 111, and 142, with up to 1, 2, 3, 4, 5, 6, 7, or 8 amino acid substitutions; the CDR-H1 is a CDR-H1 selected from any one of SEQ ID NOs: 10, 27, and 52, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 343 and 365, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 87, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 343 and 365; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175, 203, and 223, the CDR-H2 has at least about 80%, 90%, 91%, 92%, 93%, 94%, 95%, 96%, 97%, 98% or 99% identity to a CDR-H2 selected from any one of SEQ ID NOs: 87, 111, and 142, the CDR-H1 has at least about 80%, 90%, 91%, 92%, 93%, 94%, 95%, 96%, 97%, 98% or 99% identity to a CDR-H1 selected from any one of SEQ ID NOs: 10, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 175, 203, and 223, with up to 1, 2, 3, 4, 5, 6, 7, or 8 amino acid substitutions; the CDR-H2 is a CDR-H2 selected from any one of SEQ ID NOs: 87, 111, and 142, with up to 1, 2, 3, 4, 5, 6, 7, or 8 amino acid substitutions; the CDR-H1 is a CDR-H1 selected from any one of SEQ ID NOs: 10, 27, and 52, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 87, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175, 203, and 223, the CDR-H2 has at least about 80%, 90%, 91%, 92%, 93%, 94%, 95%, 96%, 97%, 98% or 99% identity to a CDR-H2 selected from any one of SEQ ID NOs: 87, 111, and 142, the CDR-H1 has at least about 80%, 90%, 91%, 92%, 93%, 94%, 95%, 96%, 97%, 98% or 99% identity to a CDR-H1 selected from any one of SEQ ID NOs: 13, 30, and 55, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 175, 203, and 223, with up to 1, 2, 3, 4, 5, 6, 7, or 8 amino acid substitutions; the CDR-H2 is a CDR-H2 selected from any one of SEQ ID NOs: 87, 111, and 142, with up to 1, 2, 3, 4, 5, 6, 7, or 8 amino acid substitutions; the CDR-H1 is a CDR-H1 selected from any one of SEQ ID NOs: 13, 30, and 55, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 13, 30, and 55; a CDR-H2 comprising an amino acid sequence set forth in any one of SEQ ID NOs: 87, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175, 203, and 223, the CDR-H2 has at least about 80%, 90%, 91%, 92%, 93%, 94%, 95%, 96%, 97%, 98% or 99% identity to a CDR-H2 selected from any one of SEQ ID NOs: 70, 111, and 142, the CDR-H1 has at least about 80%, 90%, 91%, 92%, 93%, 94%, 95%, 96%, 97%, 98% or 99% identity to a CDR-H1 selected from any one of SEQ ID NOs: 10, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 368 and 394, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 175, 203, and 223, with up to 1, 2, 3, 4, 5, 6, 7, or 8 amino acid substitutions; the CDR-H2 is a CDR-H2 selected from any one of SEQ ID NOs: 70, 111, and 142, with up to 1, 2, 3, 4, 5, 6, 7, or 8 amino acid substitutions; the CDR-H1 is a CDR-H1 selected from any one of SEQ ID NOs: 10, 27, and 52, with up to 1, 2, 3, 4, or 5 amino acid substitutions; the CDR-L3 is a CDR-L3 selected from any one of SEQ ID NOs: 368 and 394, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 368 and 394.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175, 203, and 223, the CDR-H2 has at least about 80%, 90%, 91%, 92%, 93%, 94%, 95%, 96%, 97%, 98% or 99% identity to a CDR-H2 selected from any one of SEQ ID NOs: 87, 111, and 142, the CDR-H1 has at least about 80%, 90%, 91%, 92%, 93%, 94%, 95%, 96%, 97%, 98% or 99% identity to a CDR-H1 selected from any one of SEQ ID NOs: 10, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 348 and 366, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 175, 203, and 223, with up to 1, 2, 3, 4, 5, 6, 7, or 8 amino acid substitutions; the CDR-H2 is a CDR-H2 selected from any one of SEQ ID NOs: 87, 111, and 142, with up to 1, 2, 3, 4, 5, 6, 7, or 8 amino acid substitutions; the CDR-H1 is a CDR-H1 selected from any one of SEQ ID NOs: 10, 27, and 52, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 348 and 366, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 87, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 348 and 366; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175, 203, and 223, the CDR-H2 has at least about 80%, 90%, 91%, 92%, 93%, 94%, 95%, 96%, 97%, 98% or 99% identity to a CDR-H2 selected from any one of SEQ ID NOs: 70, 111, and 142, the CDR-H1 has at least about 80%, 90%, 91%, 92%, 93%, 94%, 95%, 96%, 97%, 98% or 99% identity to a CDR-H1 selected from any one of SEQ ID NOs: 10, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 381 and 405, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 175, 203, and 223, with up to 1, 2, 3, 4, 5, 6, 7, or 8 amino acid substitutions; the CDR-H2 is a CDR-H2 selected from any one of SEQ ID NOs: 70, 111, and 142, with up to 1, 2, 3, 4, 5, 6, 7, or 8 amino acid substitutions; the CDR-H1 is a CDR-H1 selected from any one of SEQ ID NOs: 10, 27, and 52, with up to 1, 2, 3, 4, or 5 amino acid substitutions; the CDR-L3 is a CDR-L3 selected from any one of SEQ ID NOs: 381 and 405, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 381 and 405.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175, 203, and 223, the CDR-H2 has at least about 80%, 90%, 91%, 92%, 93%, 94%, 95%, 96%, 97%, 98% or 99% identity to a CDR-H2 selected from any one of SEQ ID NOs: 70, 111, and 142, the CDR-H1 has at least about 80%, 90%, 91%, 92%, 93%, 94%, 95%, 96%, 97%, 98% or 99% identity to a CDR-H1 selected from any one of SEQ ID NOs: 10, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 368 and 394, the CDR-L2 has at least about 80%, 90%, 91%, 92%, 93%, 94%, 95%, 96%, 97%, 98% or 99% identity to a CDR-L2 selected from any one of SEQ ID NOs: 335 and 359, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 175, 203, and 223, with up to 1, 2, 3, 4, 5, 6, 7, or 8 amino acid substitutions; the CDR-H2 is a CDR-H2 selected from any one of SEQ ID NOs: 70, 111, and 142, with up to 1, 2, 3, 4, 5, 6, 7, or 8 amino acid substitutions; the CDR-H1 is a CDR-H1 selected from any one of SEQ ID NOs: 10, 27, and 52, with up to 1, 2, 3, 4, or 5 amino acid substitutions; the CDR-L3 is a CDR-L3 selected from any one of SEQ ID NOs: 368 and 394, with up to 1, 2, 3, 4, or 5 amino acid substitutions; the CDR-L2 is a CDR-L2 selected from any one of SEQ ID NOs: 335 and 359, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 335 and 359; and a CDR-L3 comprising an amino acid sequence set forth in any one of SEQ ID NOs: 368 and 394.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175, 203, and 223, the CDR-H2 has at least about 80%, 90%, 91%, 92%, 93%, 94%, 95%, 96%, 97%, 98% or 99% identity to a CDR-H2 selected from any one of SEQ ID NOs: 86, 124, and 155, the CDR-H1 has at least about 80%, 90%, 91%, 92%, 93%, 94%, 95%, 96%, 97%, 98% or 99% identity to a CDR-H1 selected from any one of SEQ ID NOs: 1, 22, and 44, the CDR-L3 has at least about 80%, 90%, 91%, 92%, 93%, 94%, 95%, 96%, 97%, 98% or 99% identity to a CDR-L3 selected from any one of SEQ ID NOs: 381 and 405, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 175, 203, and 223, with up to 1, 2, 3, 4, 5, 6, 7, or 8 amino acid substitutions; the CDR-H2 is a CDR-H2 selected from any one of SEQ ID NOs: 86, 124, and 155, with up to 1, 2, 3, 4, 5, 6, 7, or 8 amino acid substitutions; the CDR-H1 is a CDR-H1 selected from any one of SEQ ID NOs: 1, 22, and 44, with up to 1, 2, 3, 4, or 5 amino acid substitutions; the CDR-L3 is a CDR-L3 selected from any one of SEQ ID NOs: 381 and 405, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 1, 22, and 44; a CDR-H2 comprising an amino acid sequence set forth in any one of SEQ ID NOs: 86, 124, and 155; a CDR-H3 comprising an amino acid sequence set forth in any one of SEQ ID NOs: 175, 203, and 223; a CDR-L1 comprising an amino acid sequence set forth in any one of SEQ ID NOs: 260, 288, or 313; a CDR-L2 comprising an amino acid sequence set forth in any one of SEQ ID NOs: 337 and 361; and a CDR-L3 comprising an amino acid sequence set forth in any one of SEQ ID NOs: 381 and 405.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175, 203, and 223, the CDR-H2 has at least about 80%, 90%, 91%, 92%, 93%, 94%, 95%, 96%, 97%, 98% or 99% identity to a CDR-H2 selected from any one of SEQ ID NOs: 86, 124, and 155, the CDR-H1 has at least about 80%, 90%, 91%, 92%, 93%, 94%, 95%, 96%, 97%, 98% or 99% identity to a CDR-H1 selected from any one of SEQ ID NOs: 1, 22, and 44, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 175, 203, and 223, with up to 1, 2, 3, 4, 5, 6, 7, or 8 amino acid substitutions; the CDR-H2 is a CDR-H2 selected from any one of SEQ ID NOs: 86, 124, and 155, with up to 1, 2, 3, 4, 5, 6, 7, or 8 amino acid substitutions; the CDR-H1 is a CDR-H1 selected from any one of SEQ ID NOs: 1, 22, and 44, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 1, 22, and 44; a CDR-H2 comprising an amino acid sequence set forth in any one of SEQ ID NOs: 86, 124, and 155; a CDR-H3 comprising an amino acid sequence set forth in any one of SEQ ID NOs: 175, 203, and 223; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 188, 203, and 239, the CDR-H2 has at least about 80%, 90%, 91%, 92%, 93%, 94%, 95%, 96%, 97%, 98% or 99% identity to a CDR-H2 selected from any one of SEQ ID NOs: 70, 111, and 142, the CDR-H1 has at least about 80%, 90%, 91%, 92%, 93%, 94%, 95%, 96%, 97%, 98% or 99% identity to a CDR-H1 selected from any one of SEQ ID NOs: 10, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 188, 203, and 239, with up to 1, 2, 3, 4, 5, 6, 7, or 8 amino acid substitutions; the CDR-H2 is a CDR-H2 selected from any one of SEQ ID NOs: 70, 111, and 142, with up to 1, 2, 3, 4, 5, 6, 7, or 8 amino acid substitutions; the CDR-H1 is a CDR-H1 selected from any one of SEQ ID NOs: 10, 27, and 52, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 188, 203, and 239; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 188, 203, and 237, the CDR-H2 has at least about 80%, 90%, 91%, 92%, 93%, 94%, 95%, 96%, 97%, 98% or 99% identity to a CDR-H2 selected from any one of SEQ ID NOs: 70, 111, and 142, the CDR-H1 has at least about 80%, 90%, 91%, 92%, 93%, 94%, 95%, 96%, 97%, 98% or 99% identity to a CDR-H1 selected from any one of SEQ ID NOs: 10, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 188, 203, and 237, with up to 1, 2, 3, 4, 5, 6, 7, or 8 amino acid substitutions; the CDR-H2 is a CDR-H2 selected from any one of SEQ ID NOs: 70, 111, and 142, with up to 1, 2, 3, 4, 5, 6, 7, or 8 amino acid substitutions; the CDR-H1 is a CDR-H1 selected from any one of SEQ ID NOs: 10, 27, and 52, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 188, 203, and 237; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 188, 203, and 237, the CDR-H2 has at least about 80%, 90%, 91%, 92%, 93%, 94%, 95%, 96%, 97%, 98% or 99% identity to a CDR-H2 selected from any one of SEQ ID NOs: 78, 119, and 150, the CDR-H1 has at least about 80%, 90%, 91%, 92%, 93%, 94%, 95%, 96%, 97%, 98% or 99% identity to a CDR-H1 selected from any one of SEQ ID NOs: 10, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 368 and 394, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 188, 203, and 237, with up to 1, 2, 3, 4, 5, 6, 7, or 8 amino acid substitutions; the CDR-H2 is a CDR-H2 selected from any one of SEQ ID NOs: 78, 119, and 150, with up to 1, 2, 3, 4, 5, 6, 7, or 8 amino acid substitutions; the CDR-H1 is a CDR-H1 selected from any one of SEQ ID NOs: 10, 27, and 52, with up to 1, 2, 3, 4, or 5 amino acid substitutions; the CDR-L3 is a CDR-L3 selected from any one of SEQ ID NOs: 368 and 394, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 78, 119, and 150; a CDR-H3 comprising an amino acid sequence set forth in any one of SEQ ID NOs: 188, 203, and 237; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 368 and 394.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 176, 204, and 224, the CDR-H2 has at least about 80%, 90%, 91%, 92%, 93%, 94%, 95%, 96%, 97%, 98% or 99% identity to a CDR-H2 selected from any one of SEQ ID NOs: 70, 111, and 142, the CDR-H1 has at least about 80%, 90%, 91%, 92%, 93%, 94%, 95%, 96%, 97%, 98% or 99% identity to a CDR-H1 selected from any one of SEQ ID NOs: 2, 23, and 45, the CDR-L3 has at least about 80%, 90%, 91%, 92%, 93%, 94%, 95%, 96%, 97%, 98% or 99% identity to a CDR-L3 selected from any one of SEQ ID NOs: 369 and 395, the CDR-L2 has at least about 80%, 90%, 91%, 92%, 93%, 94%, 95%, 96%, 97%, 98% or 99% identity to a CDR-L2 selected from any one of SEQ ID NOs: 336 and 360, and the CDR-L1 has at least about 80%, 90%, 91%, 92%, 93%, 94%, 95%, 96%, 97%, 98% or 99% identity to a CDR-L1 selected from any one of SEQ ID NOs: 259, 287, and 312. In some embodiments, the CDR-H3 is a CDR-H3 selected from any one of SEQ ID NOs: 176, 204, and 224, with up to 1, 2, 3, 4, 5, 6, 7, or 8 amino acid substitutions; the CDR-H2 is a CDR-H2 selected from any one of SEQ ID NOs: 70, 111, and 142, with up to 1, 2, 3, 4, 5, 6, 7, or 8 amino acid substitutions; the CDR-H1 is a CDR-H1 selected from any one of SEQ ID NOs: 2, 23, and 45, with up to 1, 2, 3, 4, or 5 amino acid substitutions; the CDR-L3 is a CDR-L3 selected from any one of SEQ ID NOs: 369 and 395, with up to 1, 2, 3, 4, or 5 amino acid substitutions; the CDR-L2 is a CDR-L2 selected from any one of SEQ ID NOs: 336 and 360, with up to 1, 2, 3, or 4 amino acid substitutions; and the CDR-L1 is a CDR-L1 selected from any one of SEQ ID NOs: 259, 287, and 312, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 2, 23, and 45; a CDR-H2 comprising an amino acid sequence set forth in any one of SEQ ID NOs: 70, 111, and 142; a CDR-H3 comprising an amino acid sequence set forth in any one of SEQ ID NOs: 176, 204, and 224; a CDR-L1 comprising an amino acid sequence set forth in any one of SEQ ID NOs: 259, 287, and 312; a CDR-L2 comprising an amino acid sequence set forth in any one of SEQ ID NOs: 336 and 360; and a CDR-L3 comprising an amino acid sequence set forth in any one of SEQ ID NOs: 369 and 395.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 190, 215, and 240, the CDR-H2 has at least about 80%, 90%, 91%, 92%, 93%, 94%, 95%, 96%, 97%, 98% or 99% identity to a CDR-H2 selected from any one of SEQ ID NOs: 88, 125, and 156, the CDR-H1 has at least about 80%, 90%, 91%, 92%, 93%, 94%, 95%, 96%, 97%, 98% or 99% identity to a CDR-H1 selected from any one of SEQ ID NOs: 15, 34, and 59, the CDR-L3 has at least about 80%, 90%, 91%, 92%, 93%, 94%, 95%, 96%, 97%, 98% or 99% identity to a CDR-L3 selected from any one of SEQ ID NOs: 382 and 407, the CDR-L2 has at least about 80%, 90%, 91%, 92%, 93%, 94%, 95%, 96%, 97%, 98% or 99% identity to a CDR-L2 selected from any one of SEQ ID NOs: 349 and 365, and the CDR-L1 has at least about 80%, 90%, 91%, 92%, 93%, 94%, 95%, 96%, 97%, 98% or 99% identity to a CDR-L1 selected from any one of SEQ ID NOs: 269, 288, and 313. In some embodiments, the CDR-H3 is a CDR-H3 selected from any one of SEQ ID NOs: 190, 215, and 240, with up to 1, 2, 3, 4, 5, 6, 7, or 8 amino acid substitutions; the CDR-H2 is a CDR-H2 selected from any one of SEQ ID NOs: 88, 125, and 156, with up to 1, 2, 3, 4, 5, 6, 7, or 8 amino acid substitutions; the CDR-H1 is a CDR-H1 selected from any one of SEQ ID NOs: 15, 34, and 59, with up to 1, 2, 3, 4, or 5 amino acid substitutions; the CDR-L3 is a CDR-L3 selected from any one of SEQ ID NOs: 382 and 407, with up to 1, 2, 3, 4, or 5 amino acid substitutions; the CDR-L2 is a CDR-L2 selected from any one of SEQ ID NOs: 349 and 365, with up to 1, 2, 3, or 4 amino acid substitutions; and the CDR-L1 is a CDR-L1 selected from any one of SEQ ID NOs: 269, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 15, 34, and 59; a CDR-H2 comprising an amino acid sequence set forth in any one of SEQ ID NOs: 88, 125, and 156; a CDR-H3 comprising an amino acid sequence set forth in any one of SEQ ID NOs: 190, 215, and 240; a CDR-L1 comprising an amino acid sequence set forth in any one of SEQ ID NOs: 269, 288, and 313; a CDR-L2 comprising an amino acid sequence set forth in any one of SEQ ID NOs: 349 and 365; and a CDR-L3 comprising an amino acid sequence set forth in any one of SEQ ID NOs: 382 and 407.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 178, 206, and 226, the CDR-H2 has at least about 80%, 90%, 91%, 92%, 93%, 94%, 95%, 96%, 97%, 98% or 99% identity to a CDR-H2 selected from any one of SEQ ID NOs: 89, 126, and 157, the CDR-H1 has at least about 80%, 90%, 91%, 92%, 93%, 94%, 95%, 96%, 97%, 98% or 99% identity to a CDR-H1 selected from any one of SEQ ID NOs: 4, 22, and 47, the CDR-L3 has at least about 80%, 90%, 91%, 92%, 93%, 94%, 95%, 96%, 97%, 98% or 99% identity to a CDR-L3 selected from any one of SEQ ID NOs: 371 and 397, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 178, 206, and 226, with up to 1, 2, 3, 4, 5, 6, 7, or 8 amino acid substitutions; the CDR-H2 is a CDR-H2 selected from any one of SEQ ID NOs: 89, 126, and 157, with up to 1, 2, 3, 4, 5, 6, 7, or 8 amino acid substitutions; the CDR-H1 is a CDR-H1 selected from any one of SEQ ID NOs: 4, 22, and 47, with up to 1, 2, 3, 4, or 5 amino acid substitutions; the CDR-L3 is a CDR-L3 selected from any one of SEQ ID NOs: 371 and 397, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 4, 22, and 47; a CDR-H2 comprising an amino acid sequence set forth in any one of SEQ ID NOs: 89, 126, and 157; a CDR-H3 comprising an amino acid sequence set forth in any one of SEQ ID NOs: 178, 206, and 226; a CDR-L1 comprising an amino acid sequence set forth in any one of SEQ ID NOs: 260, 288, and 313; a CDR-L2 comprising an amino acid sequence set forth in any one of SEQ ID NOs: 337 and 361; and a CDR-L3 comprising an amino acid sequence set forth in any one of SEQ ID NOs: 371 and 397.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 178, 206, and 241, the CDR-H2 has at least about 80%, 90%, 91%, 92%, 93%, 94%, 95%, 96%, 97%, 98% or 99% identity to a CDR-H2 selected from any one of SEQ ID NOs: 72, 113, and 144, the CDR-H1 has at least about 80%, 90%, 91%, 92%, 93%, 94%, 95%, 96%, 97%, 98% or 99% identity to a CDR-H1 selected from any one of SEQ ID NOs: 4, 35, and 60, the CDR-L3 has at least about 80%, 90%, 91%, 92%, 93%, 94%, 95%, 96%, 97%, 98% or 99% identity to a CDR-L3 selected from any one of SEQ ID NOs: 371 and 397, the CDR-L2 has at least about 80%, 90%, 91%, 92%, 93%, 94%, 95%, 96%, 97%, 98% or 99% identity to a CDR-L2 selected from any one of SEQ ID NOs: 350 and 361, and the CDR-L1 has at least about 80%, 90%, 91%, 92%, 93%, 94%, 95%, 96%, 97%, 98% or 99% identity to a CDR-L1 selected from any one of SEQ ID NOs: 271, 296, and 320. In some embodiments, the CDR-H3 is a CDR-H3 selected from any one of SEQ ID NOs: 178, 206, and 241, with up to 1, 2, 3, 4, 5, 6, 7, or 8 amino acid substitutions; the CDR-H2 is a CDR-H2 selected from any one of SEQ ID NOs: 72, 113, and 144, with up to 1, 2, 3, 4, 5, 6, 7, or 8 amino acid substitutions; the CDR-H1 is a CDR-H1 selected from any one of SEQ ID NOs: 4, 35, and 60, with up to 1, 2, 3, 4, or 5 amino acid substitutions; the CDR-L3 is a CDR-L3 selected from any one of SEQ ID NOs: 371 and 397, with up to 1, 2, 3, 4, or 5 amino acid substitutions; the CDR-L2 is a CDR-L2 selected from any one of SEQ ID NOs: 350 and 361, with up to 1, 2, 3, or 4 amino acid substitutions; and the CDR-L1 is a CDR-L1 selected from any one of SEQ ID NOs: 271, 296, and 320, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 4, 35, and 60; a CDR-H2 comprising an amino acid sequence set forth in any one of SEQ ID NOs: 72, 113, and 144; a CDR-H3 comprising an amino acid sequence set forth in any one of SEQ ID NOs: 178, 206, and 241; a CDR-L1 comprising an amino acid sequence set forth in any one of SEQ ID NOs: 271, 296, and 320; a CDR-L2 comprising an amino acid sequence set forth in any one of SEQ ID NOs: 350 and 361; and a CDR-L3 comprising an amino acid sequence set forth in any one of SEQ ID NOs: 371 and 397.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 178, 206, and 226, the CDR-H2 has at least about 80%, 90%, 91%, 92%, 93%, 94%, 95%, 96%, 97%, 98% or 99% identity to a CDR-H2 selected from any one of SEQ ID NOs: 90, 127, and 158, the CDR-H1 has at least about 80%, 90%, 91%, 92%, 93%, 94%, 95%, 96%, 97%, 98% or 99% identity to a CDR-H1 selected from any one of SEQ ID NOs: 4, 22, and 47, the CDR-L3 has at least about 80%, 90%, 91%, 92%, 93%, 94%, 95%, 96%, 97%, 98% or 99% identity to a CDR-L3 selected from any one of SEQ ID NOs: 371 or 397, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 261, 289, and 314. In some embodiments, the CDR-H3 is a CDR-H3 selected from any one of SEQ ID NOs: 178, 206, and 226, with up to 1, 2, 3, 4, 5, 6, 7, or 8 amino acid substitutions; the CDR-H2 is a CDR-H2 selected from any one of SEQ ID NOs: 90, 127, and 158, with up to 1, 2, 3, 4, 5, 6, 7, or 8 amino acid substitutions; the CDR-H1 is a CDR-H1 selected from any one of SEQ ID NOs: 4, 22, and 47, with up to 1, 2, 3, 4, or 5 amino acid substitutions; the CDR-L3 is a CDR-L3 selected from any one of SEQ ID NOs: 371 and 397, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 261, 289, and 314, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 4, 22, and 47; a CDR-H2 comprising an amino acid sequence set forth in any one of SEQ ID NOs: 90, 127, and 158; a CDR-H3 comprising an amino acid sequence set forth in any one of SEQ ID NOs: 178, 206, and 226; a CDR-L1 comprising an amino acid sequence set forth in any one of SEQ ID NOs: 261, 289, and 314; a CDR-L2 comprising an amino acid sequence set forth in any one of SEQ ID NOs: 337 and 361; and a CDR-L3 comprising an amino acid sequence set forth in any one of SEQ ID NOs: 371 and 397.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 191, 215, and 242, the CDR-H2 has at least about 80%, 90%, 91%, 92%, 93%, 94%, 95%, 96%, 97%, 98% or 99% identity to a CDR-H2 selected from any one of SEQ ID NOs: 91, 128, and 159, the CDR-H1 has at least about 80%, 90%, 91%, 92%, 93%, 94%, 95%, 96%, 97%, 98% or 99% identity to a CDR-H1 selected from any one of SEQ ID NOs: 16, 36, and 61, the CDR-L3 has at least about 80%, 90%, 91%, 92%, 93%, 94%, 95%, 96%, 97%, 98% or 99% identity to a CDR-L3 selected from any one of SEQ ID NOs: 382 and 407, the CDR-L2 has at least about 80%, 90%, 91%, 92%, 93%, 94%, 95%, 96%, 97%, 98% or 99% identity to a CDR-L2 selected from any one of SEQ ID NOs: 351 or 361, and the CDR-L1 has at least about 80%, 90%, 91%, 92%, 93%, 94%, 95%, 96%, 97%, 98% or 99% identity to a CDR-L1 selected from any one of SEQ ID NOs: 269, 288, and 313. In some embodiments, the CDR-H3 is a CDR-H3 selected from any one of SEQ ID NOs: 191, 215, and 242, with up to 1, 2, 3, 4, 5, 6, 7, or 8 amino acid substitutions; the CDR-H2 is a CDR-H2 selected from any one of SEQ ID NOs: 91, 128, and 159, with up to 1, 2, 3, 4, 5, 6, 7, or 8 amino acid substitutions; the CDR-H1 is a CDR-H1 selected from any one of SEQ ID NOs: 16, 36, and 61, with up to 1, 2, 3, 4, or 5 amino acid substitutions; the CDR-L3 is a CDR-L3 selected from any one of SEQ ID NOs: 382 and 407, with up to 1, 2, 3, 4, or 5 amino acid substitutions; the CDR-L2 is a CDR-L2 selected from any one of SEQ ID NOs: 351 and 361, with up to 1, 2, 3, or 4 amino acid substitutions; and the CDR-L1 is a CDR-L1 selected from any one of SEQ ID NOs: 269, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 16, 36, and 61; a CDR-H2 comprising an amino acid sequence set forth in any one of SEQ ID NOs: 91, 128, and 159; a CDR-H3 comprising an amino acid sequence set forth in any one of SEQ ID NOs: 191, 215, and 242; a CDR-L1 comprising an amino acid sequence set forth in any one of SEQ ID NOs: 269, 288, and 313; a CDR-L2 comprising an amino acid sequence set forth in any one of SEQ ID NOs: 351 and 361; and a CDR-L3 comprising an amino acid sequence set forth in any one of SEQ ID NOs: 382 and 407.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 190, 215, and 240, the CDR-H2 has at least about 80%, 90%, 91%, 92%, 93%, 94%, 95%, 96%, 97%, 98% or 99% identity to a CDR-H2 selected from any one of SEQ ID NOs: 91, 128, and 159, the CDR-H1 has at least about 80%, 90%, 91%, 92%, 93%, 94%, 95%, 96%, 97%, 98% or 99% identity to a CDR-H1 selected from any one of SEQ ID NOs: 16, 36, and 61, the CDR-L3 has at least about 80%, 90%, 91%, 92%, 93%, 94%, 95%, 96%, 97%, 98% or 99% identity to a CDR-L3 selected from any one of SEQ ID NOs: 371 and 397, the CDR-L2 has at least about 80%, 90%, 91%, 92%, 93%, 94%, 95%, 96%, 97%, 98% or 99% identity to a CDR-L2 selected from any one of SEQ ID NOs: 351 and 361, and the CDR-L1 has at least about 80%, 90%, 91%, 92%, 93%, 94%, 95%, 96%, 97%, 98% or 99% identity to a CDR-L1 selected from any one of SEQ ID NOs: 269, 288, and 313. In some embodiments, the CDR-H3 is a CDR-H3 selected from any one of SEQ ID NOs: 190, 215, and 240, with up to 1, 2, 3, 4, 5, 6, 7, or 8 amino acid substitutions; the CDR-H2 is a CDR-H2 selected from any one of SEQ ID NOs: 91, 128, and 159, with up to 1, 2, 3, 4, 5, 6, 7, or 8 amino acid substitutions; the CDR-H1 is a CDR-H1 selected from any one of SEQ ID NOs: 16, 36, and 61, with up to 1, 2, 3, 4, or 5 amino acid substitutions; the CDR-L3 is a CDR-L3 selected from any one of SEQ ID NOs: 371 and 397, with up to 1, 2, 3, 4, or 5 amino acid substitutions; the CDR-L2 is a CDR-L2 selected from any one of SEQ ID NOs: 351 and 361, with up to 1, 2, 3, or 4 amino acid substitutions; and the CDR-L1 is a CDR-L1 selected from any one of SEQ ID NOs: 269, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 16, 36, and 61; a CDR-H2 comprising an amino acid sequence set forth in any one of SEQ ID NOs: 91, 128, and 159; a CDR-H3 comprising an amino acid sequence set forth in any one of SEQ ID NOs: 190, 215, and 240; a CDR-L1 comprising an amino acid sequence set forth in any one of SEQ ID NOs: 269, 288, and 313; a CDR-L2 comprising an amino acid sequence set forth in any one of SEQ ID NOs: 351 and 361; and a CDR-L3 comprising an amino acid sequence set forth in any one of SEQ ID NOs: 371 and 397.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 190, 215, and 240, the CDR-H2 has at least about 80%, 90%, 91%, 92%, 93%, 94%, 95%, 96%, 97%, 98% or 99% identity to a CDR-H2 selected from any one of SEQ ID NOs: 72, 113, and 144, the CDR-H1 has at least about 80%, 90%, 91%, 92%, 93%, 94%, 95%, 96%, 97%, 98% or 99% identity to a CDR-H1 selected from any one of SEQ ID NOs: 15, 27, and 62, the CDR-L3 has at least about 80%, 90%, 91%, 92%, 93%, 94%, 95%, 96%, 97%, 98% or 99% identity to a CDR-L3 selected from any one of SEQ ID NOs: 382 and 407, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 269, 288, and 313. In some embodiments, the CDR-H3 is a CDR-H3 selected from any one of SEQ ID NOs: 190, 215, and 240, with up to 1, 2, 3, 4, 5, 6, 7, or 8 amino acid substitutions; the CDR-H2 is a CDR-H2 selected from any one of SEQ ID NOs: 72, 113, and 144, with up to 1, 2, 3, 4, 5, 6, 7, or 8 amino acid substitutions; the CDR-H1 is a CDR-H1 selected from any one of SEQ ID NOs: 15, 27, and 62, with up to 1, 2, 3, 4, or 5 amino acid substitutions; the CDR-L3 is a CDR-L3 selected from any one of SEQ ID NOs: 382 and 407, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 269, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 15, 27, and 62; a CDR-H2 comprising an amino acid sequence set forth in any one of SEQ ID NOs: 72, 113, and 144; a CDR-H3 comprising an amino acid sequence set forth in any one of SEQ ID NOs: 190, 215, and 240; a CDR-L1 comprising an amino acid sequence set forth in any one of SEQ ID NOs: 269, 288, and 313; a CDR-L2 comprising an amino acid sequence set forth in any one of SEQ ID NOs: 337 and 361; and a CDR-L3 comprising an amino acid sequence set forth in any one of SEQ ID NOs: 382 and 407.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 190, 215, and 240, the CDR-H2 has at least about 80%, 90%, 91%, 92%, 93%, 94%, 95%, 96%, 97%, 98% or 99% identity to a CDR-H2 selected from any one of SEQ ID NOs: 72, 113, and 144, the CDR-H1 has at least about 80%, 90%, 91%, 92%, 93%, 94%, 95%, 96%, 97%, 98% or 99% identity to a CDR-H1 selected from any one of SEQ ID NOs: 15, 27, and 62, the CDR-L3 has at least about 80%, 90%, 91%, 92%, 93%, 94%, 95%, 96%, 97%, 98% or 99% identity to a CDR-L3 selected from any one of SEQ ID NOs: 383 and 408, the CDR-L2 has at least about 80%, 90%, 91%, 92%, 93%, 94%, 95%, 96%, 97%, 98% or 99% identity to a CDR-L2 selected from any one of SEQ ID NOs: 351 and 361, and the CDR-L1 has at least about 80%, 90%, 91%, 92%, 93%, 94%, 95%, 96%, 97%, 98% or 99% identity to a CDR-L1 selected from any one of SEQ ID NOs: 269, 288, and 313. In some embodiments, the CDR-H3 is a CDR-H3 selected from any one of SEQ ID NOs: 190, 215, and 240, with up to 1, 2, 3, 4, 5, 6, 7, or 8 amino acid substitutions; the CDR-H2 is a CDR-H2 selected from any one of SEQ ID NOs: 72, 113, and 144, with up to 1, 2, 3, 4, 5, 6, 7, or 8 amino acid substitutions; the CDR-H1 is a CDR-H1 selected from any one of SEQ ID NOs: 15, 27, and 62, with up to 1, 2, 3, 4, or 5 amino acid substitutions; the CDR-L3 is a CDR-L3 selected from any one of SEQ ID NOs: 383 and 408, with up to 1, 2, 3, 4, or 5 amino acid substitutions; the CDR-L2 is a CDR-L2 selected from any one of SEQ ID NOs: 351 and 361, with up to 1, 2, 3, or 4 amino acid substitutions; and the CDR-L1 is a CDR-L1 selected from any one of SEQ ID NOs: 269, 288, and 313, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 15, 27, and 62; a CDR-H2 comprising an amino acid sequence set forth in any one of SEQ ID NOs: 72, 113, and 144; a CDR-H3 comprising an amino acid sequence set forth in any one of SEQ ID NOs: 190, 215, and 240; a CDR-L1 comprising an amino acid sequence set forth in any one of SEQ ID NOs: 269, 288, and 313; a CDR-L2 comprising an amino acid sequence set forth in any one of SEQ ID NOs: 351 and 361; and a CDR-L3 comprising an amino acid sequence set forth in any one of SEQ ID NOs: 383 and 408.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 179, 207, and 227, the CDR-H2 has at least about 80%, 90%, 91%, 92%, 93%, 94%, 95%, 96%, 97%, 98% or 99% identity to a CDR-H2 selected from any one of SEQ ID NOs: 92, 129, and 160, the CDR-H1 has at least about 80%, 90%, 91%, 92%, 93%, 94%, 95%, 96%, 97%, 98% or 99% identity to a CDR-H1 selected from any one of SEQ ID NOs: 5, 37, and 63, the CDR-L3 has at least about 80%, 90%, 91%, 92%, 93%, 94%, 95%, 96%, 97%, 98% or 99% identity to a CDR-L3 selected from any one of SEQ ID NOs: 384 and 409, the CDR-L2 has at least about 80%, 90%, 91%, 92%, 93%, 94%, 95%, 96%, 97%, 98% or 99% identity to a CDR-L2 selected from any one of SEQ ID NOs: 338 and 362, and the CDR-L1 has at least about 80%, 90%, 91%, 92%, 93%, 94%, 95%, 96%, 97%, 98% or 99% identity to a CDR-L1 selected from any one of SEQ ID NOs: 272, 297, and 321. In some embodiments, the CDR-H3 is a CDR-H3 selected from any one of SEQ ID NOs: 179, 207, and 227, with up to 1, 2, 3, 4, 5, 6, 7, or 8 amino acid substitutions; the CDR-H2 is a CDR-H2 selected from any one of SEQ ID NOs: 92, 129, and 160, with up to 1, 2, 3, 4, 5, 6, 7, or 8 amino acid substitutions; the CDR-H1 is a CDR-H1 selected from any one of SEQ ID NOs: 5, 37, and 63, with up to 1, 2, 3, 4, or 5 amino acid substitutions; the CDR-L3 is a CDR-L3 selected from any one of SEQ ID NOs: 384 and 409, with up to 1, 2, 3, 4, or 5 amino acid substitutions; the CDR-L2 is a CDR-L2 selected from any one of SEQ ID NOs: 338 and 362, with up to 1, 2, 3, or 4 amino acid substitutions; and the CDR-L1 is a CDR-L1 selected from any one of SEQ ID NOs: 272, 297, and 321, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 5, 37, and 63; a CDR-H2 comprising an amino acid sequence set forth in any one of SEQ ID NOs: 92, 129, and 160; a CDR-H3 comprising an amino acid sequence set forth in any one of SEQ ID NOs: 179, 207, and 227; a CDR-L1 comprising an amino acid sequence set forth in any one of SEQ ID NOs: 272, 297, and 321; a CDR-L2 comprising an amino acid sequence set forth in any one of SEQ ID NOs: 338 and 362; and a CDR-L3 comprising an amino acid sequence set forth in any one of SEQ ID NOs: 384 and 409.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 179, 207, and 227, the CDR-H2 has at least about 80%, 90%, 91%, 92%, 93%, 94%, 95%, 96%, 97%, 98% or 99% identity to a CDR-H2 selected from any one of SEQ ID NOs: 93, 130, and 161, the CDR-H1 has at least about 80%, 90%, 91%, 92%, 93%, 94%, 95%, 96%, 97%, 98% or 99% identity to a CDR-H1 selected from any one of SEQ ID NOs: 5, 24, and 49, the CDR-L3 has at least about 80%, 90%, 91%, 92%, 93%, 94%, 95%, 96%, 97%, 98% or 99% identity to a CDR-L3 selected from any one of SEQ ID NOs: 384 and 409, the CDR-L2 has at least about 80%, 90%, 91%, 92%, 93%, 94%, 95%, 96%, 97%, 98% or 99% identity to a CDR-L2 selected from any one of SEQ ID NOs: 338 and 362, and the CDR-L1 has at least about 80%, 90%, 91%, 92%, 93%, 94%, 95%, 96%, 97%, 98% or 99% identity to a CDR-L1 selected from any one of SEQ ID NOs: 272, 297, and 321. In some embodiments, the CDR-H3 is a CDR-H3 selected from any one of SEQ ID NOs: 179, 207, and 227, with up to 1, 2, 3, 4, 5, 6, 7, or 8 amino acid substitutions; the CDR-H2 is a CDR-H2 selected from any one of SEQ ID NOs: 93, 130, and 161, with up to 1, 2, 3, 4, 5, 6, 7, or 8 amino acid substitutions; the CDR-H1 is a CDR-H1 selected from any one of SEQ ID NOs: 5, 24, and 49, with up to 1, 2, 3, 4, or 5 amino acid substitutions; the CDR-L3 is a CDR-L3 selected from any one of SEQ ID NOs: 384 and 409, with up to 1, 2, 3, 4, or 5 amino acid substitutions; the CDR-L2 is a CDR-L2 selected from any one of SEQ ID NOs: 338 and 362, with up to 1, 2, 3, or 4 amino acid substitutions; and the CDR-L1 is a CDR-L1 selected from any one of SEQ ID NOs: 272, 297, and 321, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 5, 24, and 49; a CDR-H2 comprising an amino acid sequence set forth in any one of SEQ ID NOs: 93, 130, and 161; a CDR-H3 comprising an amino acid sequence set forth in any one of SEQ ID NOs: 179, 207, and 227; a CDR-L1 comprising an amino acid sequence set forth in any one of SEQ ID NOs: 272, 297, and 321; a CDR-L2 comprising an amino acid sequence set forth in any one of SEQ ID NOs: 338 and 362; and a CDR-L3 comprising an amino acid sequence set forth in any one of SEQ ID NOs: 384 and 409.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 192, 207, and 243, the CDR-H2 has at least about 80%, 90%, 91%, 92%, 93%, 94%, 95%, 96%, 97%, 98% or 99% identity to a CDR-H2 selected from any one of SEQ ID NOs: 94, 131, and 162, the CDR-H1 has at least about 80%, 90%, 91%, 92%, 93%, 94%, 95%, 96%, 97%, 98% or 99% identity to a CDR-H1 selected from any one of SEQ ID NOs: 5, 840, and 48, the CDR-L3 has at least about 80%, 90%, 91%, 92%, 93%, 94%, 95%, 96%, 97%, 98% or 99% identity to a CDR-L3 selected from any one of SEQ ID NOs: 385 and 410, the CDR-L2 has at least about 80%, 90%, 91%, 92%, 93%, 94%, 95%, 96%, 97%, 98% or 99% identity to a CDR-L2 selected from any one of SEQ ID NOs: 338 and 362, and the CDR-L1 has at least about 80%, 90%, 91%, 92%, 93%, 94%, 95%, 96%, 97%, 98% or 99% identity to a CDR-L1 selected from any one of SEQ ID NOs: 273, 298, and 322. In some embodiments, the CDR-H3 is a CDR-H3 selected from any one of SEQ ID NOs: 192, 207, and 243, with up to 1, 2, 3, 4, 5, 6, 7, or 8 amino acid substitutions; the CDR-H2 is a CDR-H2 selected from any one of SEQ ID NOs: 94, 131, and 162, with up to 1, 2, 3, 4, 5, 6, 7, or 8 amino acid substitutions; the CDR-H1 is a CDR-H1 selected from any one of SEQ ID NOs: 5, 840, and 48, with up to 1, 2, 3, 4, or 5 amino acid substitutions; the CDR-L3 is a CDR-L3 selected from any one of SEQ ID NOs: 385 and 410, with up to 1, 2, 3, 4, or 5 amino acid substitutions; the CDR-L2 is a CDR-L2 selected from any one of SEQ ID NOs: 338 and 362, with up to 1, 2, 3, or 4 amino acid substitutions; and the CDR-L1 is a CDR-L1 selected from any one of SEQ ID NOs: 273, 298, and 322, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 94, 131, and 162; a CDR-H3 comprising an amino acid sequence set forth in any one of SEQ ID NOs: 192, 207, and 243; a CDR-L1 comprising an amino acid sequence set forth in any one of SEQ ID NOs: 273, 298, and 322; a CDR-L2 comprising an amino acid sequence set forth in any one of SEQ ID NOs: 338 and 362; and a CDR-L3 comprising an amino acid sequence set forth in any one of SEQ ID NOs: 385 and 410.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 179, 207, and 227, the CDR-H2 has at least about 80%, 90%, 91%, 92%, 93%, 94%, 95%, 96%, 97%, 98% or 99% identity to a CDR-H2 selected from any one of SEQ ID NOs: 95, 132, and 163, the CDR-H1 has at least about 80%, 90%, 91%, 92%, 93%, 94%, 95%, 96%, 97%, 98% or 99% identity to a CDR-H1 selected from any one of SEQ ID NOs: 17, 38, and 64, the CDR-L3 has at least about 80%, 90%, 91%, 92%, 93%, 94%, 95%, 96%, 97%, 98% or 99% identity to a CDR-L3 selected from any one of SEQ ID NOs: 386 and 411, the CDR-L2 has at least about 80%, 90%, 91%, 92%, 93%, 94%, 95%, 96%, 97%, 98% or 99% identity to a CDR-L2 selected from any one of SEQ ID NOs: 338 and 362, and the CDR-L1 has at least about 80%, 90%, 91%, 92%, 93%, 94%, 95%, 96%, 97%, 98% or 99% identity to a CDR-L1 selected from any one of SEQ ID NOs: 272, 297, and 321. In some embodiments, the CDR-H3 is a CDR-H3 selected from any one of SEQ ID NOs: 179, 207, and 227, with up to 1, 2, 3, 4, 5, 6, 7, or 8 amino acid substitutions; the CDR-H2 is a CDR-H2 selected from any one of SEQ ID NOs: 95, 132, and 163, with up to 1, 2, 3, 4, 5, 6, 7, or 8 amino acid substitutions; the CDR-H1 is a CDR-H1 selected from any one of SEQ ID NOs: 17, 38, and 64, with up to 1, 2, 3, 4, or 5 amino acid substitutions; the CDR-L3 is a CDR-L3 selected from any one of SEQ ID NOs: 386 and 411, with up to 1, 2, 3, 4, or 5 amino acid substitutions; the CDR-L2 is a CDR-L2 selected from any one of SEQ ID NOs: 338 and 362, with up to 1, 2, 3, or 4 amino acid substitutions; and the CDR-L1 is a CDR-L1 selected from any one of SEQ ID NOs: 272, 297, and 321, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 17, 38, and 64; a CDR-H2 comprising an amino acid sequence set forth in any one of SEQ ID NOs: 95, 132, and 163; a CDR-H3 comprising an amino acid sequence set forth in any one of SEQ ID NOs: 179, 207, and 227; a CDR-L1 comprising an amino acid sequence set forth in any one of SEQ ID NOs: 272, 297, and 321; a CDR-L2 comprising an amino acid sequence set forth in any one of SEQ ID NOs: 338 and 362; and a CDR-L3 comprising an amino acid sequence set forth in any one of SEQ ID NOs: 386 and 411.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 192, 207, and 243, the CDR-H2 has at least about 80%, 90%, 91%, 92%, 93%, 94%, 95%, 96%, 97%, 98% or 99% identity to a CDR-H2 selected from any one of SEQ ID NOs: 96, 130, and 164, the CDR-H1 has at least about 80%, 90%, 91%, 92%, 93%, 94%, 95%, 96%, 97%, 98% or 99% identity to a CDR-H1 selected from any one of SEQ ID NOs: 5, 24, and 49, the CDR-L3 has at least about 80%, 90%, 91%, 92%, 93%, 94%, 95%, 96%, 97%, 98% or 99% identity to a CDR-L3 selected from any one of SEQ ID NOs: 386 and 411, the CDR-L2 has at least about 80%, 90%, 91%, 92%, 93%, 94%, 95%, 96%, 97%, 98% or 99% identity to a CDR-L2 selected from any one of SEQ ID NOs: 338 and 362, and the CDR-L1 has at least about 80%, 90%, 91%, 92%, 93%, 94%, 95%, 96%, 97%, 98% or 99% identity to a CDR-L1 selected from any one of SEQ ID NOs: 274, 299, and 323. In some embodiments, the CDR-H3 is a CDR-H3 selected from any one of SEQ ID NOs: 192, 207, and 243, with up to 1, 2, 3, 4, 5, 6, 7, or 8 amino acid substitutions; the CDR-H2 is a CDR-H2 selected from any one of SEQ ID NOs: 96, 130, and 164, with up to 1, 2, 3, 4, 5, 6, 7, or 8 amino acid substitutions; the CDR-H1 is a CDR-H1 selected from any one of SEQ ID NOs: 5, 24, and 49, with up to 1, 2, 3, 4, or 5 amino acid substitutions; the CDR-L3 is a CDR-L3 selected from any one of SEQ ID NOs: 386 and 411, with up to 1, 2, 3, 4, or 5 amino acid substitutions; the CDR-L2 is a CDR-L2 selected from any one of SEQ ID NOs: 338 and 362, with up to 1, 2, 3, or 4 amino acid substitutions; and the CDR-L1 is a CDR-L1 selected from any one of SEQ ID NOs: 274, 299, and 323, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 5, 24, and 49; a CDR-H2 comprising an amino acid sequence set forth in any one of SEQ ID NOs: 96, 130, and 164; a CDR-H3 comprising an amino acid sequence set forth in any one of SEQ ID NOs: 192, 207, and 243; a CDR-L1 comprising an amino acid sequence set forth in any one of SEQ ID NOs: 274, 299, and 323; a CDR-L2 comprising an amino acid sequence set forth in any one of SEQ ID NOs: 338 and 362; and a CDR-L3 comprising an amino acid sequence set forth in any one of SEQ ID NOs: 386 and 411.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 179, 207, and 227, the CDR-H2 has at least about 80%, 90%, 91%, 92%, 93%, 94%, 95%, 96%, 97%, 98% or 99% identity to a CDR-H2 selected from any one of SEQ ID NOs: 97, 130, and 164, the CDR-H1 has at least about 80%, 90%, 91%, 92%, 93%, 94%, 95%, 96%, 97%, 98% or 99% identity to a CDR-H1 selected from any one of SEQ ID NOs: 5, 24, and 49, the CDR-L3 has at least about 80%, 90%, 91%, 92%, 93%, 94%, 95%, 96%, 97%, 98% or 99% identity to a CDR-L3 selected from any one of SEQ ID NOs: 386 and 411, the CDR-L2 has at least about 80%, 90%, 91%, 92%, 93%, 94%, 95%, 96%, 97%, 98% or 99% identity to a CDR-L2 selected from any one of SEQ ID NOs: 338 and 362, and the CDR-L1 has at least about 80%, 90%, 91%, 92%, 93%, 94%, 95%, 96%, 97%, 98% or 99% identity to a CDR-L1 selected from any one of SEQ ID NOs: 275, 300, and 324. In some embodiments, the CDR-H3 is a CDR-H3 selected from any one of SEQ ID NOs: 179, 207, and 227, with up to 1, 2, 3, 4, 5, 6, 7, or 8 amino acid substitutions; the CDR-H2 is a CDR-H2 selected from any one of SEQ ID NOs: 97, 130, and 164, with up to 1, 2, 3, 4, 5, 6, 7, or 8 amino acid substitutions; the CDR-H1 is a CDR-H1 selected from any one of SEQ ID NOs: 5, 24, and 49, with up to 1, 2, 3, 4, or 5 amino acid substitutions; the CDR-L3 is a CDR-L3 selected from any one of SEQ ID NOs: 386 and 411, with up to 1, 2, 3, 4, or 5 amino acid substitutions; the CDR-L2 is a CDR-L2 selected from any one of SEQ ID NOs: 338 and 362, with up to 1, 2, 3, or 4 amino acid substitutions; and the CDR-L1 is a CDR-L1 selected from any one of SEQ ID NOs: 275, 300, and 324, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 5, 24, and 49; a CDR-H2 comprising an amino acid sequence set forth in any one of SEQ ID NOs: 97, 130, and 164; a CDR-H3 comprising an amino acid sequence set forth in any one of SEQ ID NOs: 179, 207, and 227; a CDR-L1 comprising an amino acid sequence set forth in any one of SEQ ID NOs: 275, 300, and 324; a CDR-L2 comprising an amino acid sequence set forth in any one of SEQ ID NOs: 338 and 362; and a CDR-L3 comprising an amino acid sequence set forth in any one of SEQ ID NOs: 386 and 411.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 193, 216, and 244, the CDR-H2 has at least about 80%, 90%, 91%, 92%, 93%, 94%, 95%, 96%, 97%, 98% or 99% identity to a CDR-H2 selected from any one of SEQ ID NOs: 98, 116, and 147, the CDR-H1 has at least about 80%, 90%, 91%, 92%, 93%, 94%, 95%, 96%, 97%, 98% or 99% identity to a CDR-H1 selected from any one of SEQ ID NOs: 18, 39, and 65, the CDR-L3 has at least about 80%, 90%, 91%, 92%, 93%, 94%, 95%, 96%, 97%, 98% or 99% identity to a CDR-L3 selected from any one of SEQ ID NOs: 387 and 412, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 276, 301, and 325. In some embodiments, the CDR-H3 is a CDR-H3 selected from any one of SEQ ID NOs: 193, 216, and 244, with up to 1, 2, 3, 4, 5, 6, 7, or 8 amino acid substitutions; the CDR-H2 is a CDR-H2 selected from any one of SEQ ID NOs: 98, 116, and 147, with up to 1, 2, 3, 4, 5, 6, 7, or 8 amino acid substitutions; the CDR-H1 is a CDR-H1 selected from any one of SEQ ID NOs: 18, 39, and 65, with up to 1, 2, 3, 4, or 5 amino acid substitutions; the CDR-L3 is a CDR-L3 selected from any one of SEQ ID NOs: 387 and 412, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 276, 301, and 325, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 18, 39, and 65; a CDR-H2 comprising an amino acid sequence set forth in any one of SEQ ID NOs: 98, 116, and 147; a CDR-H3 comprising an amino acid sequence set forth in any one of SEQ ID NOs: 193, 216, and 244; a CDR-L1 comprising an amino acid sequence set forth in any one of SEQ ID NOs: 276, 301, and 325; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 193, 216, and 245, the CDR-H2 has at least about 80%, 90%, 91%, 92%, 93%, 94%, 95%, 96%, 97%, 98% or 99% identity to a CDR-H2 selected from any one of SEQ ID NOs: 98, 116, and 147, the CDR-H1 has at least about 80%, 90%, 91%, 92%, 93%, 94%, 95%, 96%, 97%, 98% or 99% identity to a CDR-H1 selected from any one of SEQ ID NOs: 18, 39, and 65, the CDR-L3 has at least about 80%, 90%, 91%, 92%, 93%, 94%, 95%, 96%, 97%, 98% or 99% identity to a CDR-L3 selected from any one of SEQ ID NOs: 387 and 412, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 277, 302, and 326. In some embodiments, the CDR-H3 is a CDR-H3 selected from any one of SEQ ID NOs: 193, 216, and 245, with up to 1, 2, 3, 4, 5, 6, 7, or 8 amino acid substitutions; the CDR-H2 is a CDR-H2 selected from any one of SEQ ID NOs: 98, 116, and 147, with up to 1, 2, 3, 4, 5, 6, 7, or 8 amino acid substitutions; the CDR-H1 is a CDR-H1 selected from any one of SEQ ID NOs: 18, 39, and 65, with up to 1, 2, 3, 4, or 5 amino acid substitutions; the CDR-L3 is a CDR-L3 selected from any one of SEQ ID NOs: 387 and 412, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 277, 302, and 326, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 18, 39, and 65; a CDR-H2 comprising an amino acid sequence set forth in any one of SEQ ID NOs: 98, 116, and 147; a CDR-H3 comprising an amino acid sequence set forth in any one of SEQ ID NOs: 193, 216, and 245; a CDR-L1 comprising an amino acid sequence set forth in any one of SEQ ID NOs: 277, 302, and 326; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 194, 217, and 246, the CDR-H2 has at least about 80%, 90%, 91%, 92%, 93%, 94%, 95%, 96%, 97%, 98% or 99% identity to a CDR-H2 selected from any one of SEQ ID NOs: 98, 116, and 147, the CDR-H1 has at least about 80%, 90%, 91%, 92%, 93%, 94%, 95%, 96%, 97%, 98% or 99% identity to a CDR-H1 selected from any one of SEQ ID NOs: 18, 39, and 65, the CDR-L3 has at least about 80%, 90%, 91%, 92%, 93%, 94%, 95%, 96%, 97%, 98% or 99% identity to a CDR-L3 selected from any one of SEQ ID NOs: 387 and 412, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 276, 301, and 325. In some embodiments, the CDR-H3 is a CDR-H3 selected from any one of SEQ ID NOs: 194, 217, and 246, with up to 1, 2, 3, 4, 5, 6, 7, or 8 amino acid substitutions; the CDR-H2 is a CDR-H2 selected from any one of SEQ ID NOs: 98, 116, and 147, with up to 1, 2, 3, 4, 5, 6, 7, or 8 amino acid substitutions; the CDR-H1 is a CDR-H1 selected from any one of SEQ ID NOs: 18, 39, and 65, with up to 1, 2, 3, 4, or 5 amino acid substitutions; the CDR-L3 is a CDR-L3 selected from any one of SEQ ID NOs: 387 and 412, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 276, 301, and 325, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 18, 39, and 65; a CDR-H2 comprising an amino acid sequence set forth in any one of SEQ ID NOs: 98, 116, and 147; a CDR-H3 comprising an amino acid sequence set forth in any one of SEQ ID NOs: 194, 217, and 246; a CDR-L1 comprising an amino acid sequence set forth in any one of SEQ ID NOs: 276, 301, and 325; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 194, 217, and 247, the CDR-H2 has at least about 80%, 90%, 91%, 92%, 93%, 94%, 95%, 96%, 97%, 98% or 99% identity to a CDR-H2 selected from any one of SEQ ID NOs: 98, 116, and 147, the CDR-H1 has at least about 80%, 90%, 91%, 92%, 93%, 94%, 95%, 96%, 97%, 98% or 99% identity to a CDR-H1 selected from any one of SEQ ID NOs: 18, 39, and 65, the CDR-L3 has at least about 80%, 90%, 91%, 92%, 93%, 94%, 95%, 96%, 97%, 98% or 99% identity to a CDR-L3 selected from any one of SEQ ID NOs: 388 and 413, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 276, 301, and 325. In some embodiments, the CDR-H3 is a CDR-H3 selected from any one of SEQ ID NOs: 194, 217, and 247, with up to 1, 2, 3, 4, 5, 6, 7, or 8 amino acid substitutions; the CDR-H2 is a CDR-H2 selected from any one of SEQ ID NOs: 98, 116, and 147, with up to 1, 2, 3, 4, 5, 6, 7, or 8 amino acid substitutions; the CDR-H1 is a CDR-H1 selected from any one of SEQ ID NOs: 18, 39, and 65, with up to 1, 2, 3, 4, or 5 amino acid substitutions; the CDR-L3 is a CDR-L3 selected from any one of SEQ ID NOs: 388 and 413, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 276, 301, and 325, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 18, 39, and 65; a CDR-H2 comprising an amino acid sequence set forth in any one of SEQ ID NOs: 98, 116, and 147; a CDR-H3 comprising an amino acid sequence set forth in any one of SEQ ID NOs: 194, 217, and 247; a CDR-L1 comprising an amino acid sequence set forth in any one of SEQ ID NOs: 276, 301, and 325; a CDR-L2 comprising an amino acid sequence set forth in any one of SEQ ID NOs: 337 and 361; and a CDR-L3 comprising an amino acid sequence set forth in any one of SEQ ID NOs: 388 and 413.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 180, 208, and 248, the CDR-H2 has at least about 80%, 90%, 91%, 92%, 93%, 94%, 95%, 96%, 97%, 98% or 99% identity to a CDR-H2 selected from any one of SEQ ID NOs: 99, 133, and 165, the CDR-H1 has at least about 80%, 90%, 91%, 92%, 93%, 94%, 95%, 96%, 97%, 98% or 99% identity to a CDR-H1 selected from any one of SEQ ID NOs: 19, 40, and 66, the CDR-L3 has at least about 80%, 90%, 91%, 92%, 93%, 94%, 95%, 96%, 97%, 98% or 99% identity to a CDR-L3 selected from any one of SEQ ID NOs: 387 and 412, the CDR-L2 has at least about 80%, 90%, 91%, 92%, 93%, 94%, 95%, 96%, 97%, 98% or 99% identity to a CDR-L2 selected from any one of SEQ ID NOs: 352 and 367, and the CDR-L1 has at least about 80%, 90%, 91%, 92%, 93%, 94%, 95%, 96%, 97%, 98% or 99% identity to a CDR-L1 selected from any one of SEQ ID NOs: 278, 303, and 327. In some embodiments, the CDR-H3 is a CDR-H3 selected from any one of SEQ ID NOs: 180, 208, and 248, with up to 1, 2, 3, 4, 5, 6, 7, or 8 amino acid substitutions; the CDR-H2 is a CDR-H2 selected from any one of SEQ ID NOs: 99, 133, and 165, with up to 1, 2, 3, 4, 5, 6, 7, or 8 amino acid substitutions; the CDR-H1 is a CDR-H1 selected from any one of SEQ ID NOs: 19, 40, and 66, with up to 1, 2, 3, 4, or 5 amino acid substitutions; the CDR-L3 is a CDR-L3 selected from any one of SEQ ID NOs: 387 and 412, with up to 1, 2, 3, 4, or 5 amino acid substitutions; the CDR-L2 is a CDR-L2 selected from any one of SEQ ID NOs: 352 and 367, with up to 1, 2, 3, or 4 amino acid substitutions; and the CDR-L1 is a CDR-L1 selected from any one of SEQ ID NOs: 278, 303, and 327, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 19, 40, and 66; a CDR-H2 comprising an amino acid sequence set forth in any one of SEQ ID NOs: 99, 133, and 165; a CDR-H3 comprising an amino acid sequence set forth in any one of SEQ ID NOs: 180, 208, and 248; a CDR-L1 comprising an amino acid sequence set forth in any one of SEQ ID NOs: 278, 303, and 327; a CDR-L2 comprising an amino acid sequence set forth in any one of SEQ ID NOs: 352 and 367; and a CDR-L3 comprising an amino acid sequence set forth in any one of SEQ ID NOs: 387 and 412.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 195, 218, and 249, the CDR-H2 has at least about 80%, 90%, 91%, 92%, 93%, 94%, 95%, 96%, 97%, 98% or 99% identity to a CDR-H2 selected from any one of SEQ ID NOs: 100, 134, and 166, the CDR-H1 has at least about 80%, 90%, 91%, 92%, 93%, 94%, 95%, 96%, 97%, 98% or 99% identity to a CDR-H1 selected from any one of SEQ ID NOs: 20, 41, and 67, the CDR-L3 has at least about 80%, 90%, 91%, 92%, 93%, 94%, 95%, 96%, 97%, 98% or 99% identity to a CDR-L3 selected from any one of SEQ ID NOs: 374 and 400, the CDR-L2 has at least about 80%, 90%, 91%, 92%, 93%, 94%, 95%, 96%, 97%, 98% or 99% identity to a CDR-L2 selected from any one of SEQ ID NOs: 353 and 361, and the CDR-L1 has at least about 80%, 90%, 91%, 92%, 93%, 94%, 95%, 96%, 97%, 98% or 99% identity to a CDR-L1 selected from any one of SEQ ID NOs: 279, 304, and 328. In some embodiments, the CDR-H3 is a CDR-H3 selected from any one of SEQ ID NOs: 195, 218, and 249, with up to 1, 2, 3, 4, 5, 6, 7, or 8 amino acid substitutions; the CDR-H2 is a CDR-H2 selected from any one of SEQ ID NOs: 100, 134, and 166, with up to 1, 2, 3, 4, 5, 6, 7, or 8 amino acid substitutions; the CDR-H1 is a CDR-H1 selected from any one of SEQ ID NOs: 20, 41, and 67, with up to 1, 2, 3, 4, or 5 amino acid substitutions; the CDR-L3 is a CDR-L3 selected from any one of SEQ ID NOs: 374 and 400, with up to 1, 2, 3, 4, or 5 amino acid substitutions; the CDR-L2 is a CDR-L2 selected from any one of SEQ ID NOs: 353 and 361, with up to 1, 2, 3, or 4 amino acid substitutions; and the CDR-L1 is a CDR-L1 selected from any one of SEQ ID NOs: 279, 304, and 328, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 20, 41, and 67; a CDR-H2 comprising an amino acid sequence set forth in any one of SEQ ID NOs: 100, 134, and 166; a CDR-H3 comprising an amino acid sequence set forth in any one of SEQ ID NOs: 195, 218, and 249; a CDR-L1 comprising an amino acid sequence set forth in any one of SEQ ID NOs: 279, 304, and 328; a CDR-L2 comprising an amino acid sequence set forth in any one of SEQ ID NOs: 353 and 361; and a CDR-L3 comprising an amino acid sequence set forth in any one of SEQ ID NOs: 374 and 400.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 180, 208, and 248, the CDR-H2 has at least about 80%, 90%, 91%, 92%, 93%, 94%, 95%, 96%, 97%, 98% or 99% identity to a CDR-H2 selected from any one of SEQ ID NOs: 101, 133, and 167, the CDR-H1 has at least about 80%, 90%, 91%, 92%, 93%, 94%, 95%, 96%, 97%, 98% or 99% identity to a CDR-H1 selected from any one of SEQ ID NOs: 20, 840, and 48, the CDR-L3 has at least about 80%, 90%, 91%, 92%, 93%, 94%, 95%, 96%, 97%, 98% or 99% identity to a CDR-L3 selected from any one of SEQ ID NOs: 389 and 414, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 280, 305, and 329. In some embodiments, the CDR-H3 is a CDR-H3 selected from any one of SEQ ID NOs: 180, 208, and 248, with up to 1, 2, 3, 4, 5, 6, 7, or 8 amino acid substitutions; the CDR-H2 is a CDR-H2 selected from any one of SEQ ID NOs: 101, 133, and 167, with up to 1, 2, 3, 4, 5, 6, 7, or 8 amino acid substitutions; the CDR-H1 is a CDR-H1 selected from any one of SEQ ID NOs: 20, 840, and 48, with up to 1, 2, 3, 4, or 5 amino acid substitutions; the CDR-L3 is a CDR-L3 selected from any one of SEQ ID NOs: 389 and 414, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 280, 305, and 329, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 20, 840, and 48; a CDR-H2 comprising an amino acid sequence set forth in any one of SEQ ID NOs: 101, 133, and 167; a CDR-H3 comprising an amino acid sequence set forth in any one of SEQ ID NOs: 180, 208, and 248; a CDR-L1 comprising an amino acid sequence set forth in any one of SEQ ID NOs: 280, 305, and 329; a CDR-L2 comprising an amino acid sequence set forth in any one of SEQ ID NOs: 337 and 361; and a CDR-L3 comprising an amino acid sequence set forth in any one of SEQ ID NOs: 389 and 414.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 180, 208, and 248, the CDR-H2 has at least about 80%, 90%, 91%, 92%, 93%, 94%, 95%, 96%, 97%, 98% or 99% identity to a CDR-H2 selected from any one of SEQ ID NOs: 102, 135, and 168, the CDR-H1 has at least about 80%, 90%, 91%, 92%, 93%, 94%, 95%, 96%, 97%, 98% or 99% identity to a CDR-H1 selected from any one of SEQ ID NOs: 5, 840, and 48, the CDR-L3 has at least about 80%, 90%, 91%, 92%, 93%, 94%, 95%, 96%, 97%, 98% or 99% identity to a CDR-L3 selected from any one of SEQ ID NOs: 389 and 414, the CDR-L2 has at least about 80%, 90%, 91%, 92%, 93%, 94%, 95%, 96%, 97%, 98% or 99% identity to a CDR-L2 selected from any one of SEQ ID NOs: 350 and 361, and the CDR-L1 has at least about 80%, 90%, 91%, 92%, 93%, 94%, 95%, 96%, 97%, 98% or 99% identity to a CDR-L1 selected from any one of SEQ ID NOs: 280, 305, and 329. In some embodiments, the CDR-H3 is a CDR-H3 selected from any one of SEQ ID NOs: 180, 208, and 248, with up to 1, 2, 3, 4, 5, 6, 7, or 8 amino acid substitutions; the CDR-H2 is a CDR-H2 selected from any one of SEQ ID NOs: 102, 135, and 168, with up to 1, 2, 3, 4, 5, 6, 7, or 8 amino acid substitutions; the CDR-H1 is a CDR-H1 selected from any one of SEQ ID NOs: 5, 840, and 48, with up to 1, 2, 3, 4, or 5 amino acid substitutions; the CDR-L3 is a CDR-L3 selected from any one of SEQ ID NOs: 389 and 414, with up to 1, 2, 3, 4, or 5 amino acid substitutions; the CDR-L2 is a CDR-L2 selected from any one of SEQ ID NOs: 350 and 361, with up to 1, 2, 3, or 4 amino acid substitutions; and the CDR-L1 is a CDR-L1 selected from any one of SEQ ID NOs: 280, 305, and 329, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 102, 135, and 168; a CDR-H3 comprising an amino acid sequence set forth in any one of SEQ ID NOs: 180, 208, and 248; a CDR-L1 comprising an amino acid sequence set forth in any one of SEQ ID NOs: 280, 305, and 329; a CDR-L2 comprising an amino acid sequence set forth in any one of SEQ ID NOs: 350 and 361; and a CDR-L3 comprising an amino acid sequence set forth in any one of SEQ ID NOs: 389 and 414.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 196, 218, and 250, the CDR-H2 has at least about 80%, 90%, 91%, 92%, 93%, 94%, 95%, 96%, 97%, 98% or 99% identity to a CDR-H2 selected from any one of SEQ ID NOs: 103, 136, and 169, the CDR-H1 has at least about 80%, 90%, 91%, 92%, 93%, 94%, 95%, 96%, 97%, 98% or 99% identity to a CDR-H1 selected from any one of SEQ ID NOs: 5, 840, and 48, the CDR-L3 has at least about 80%, 90%, 91%, 92%, 93%, 94%, 95%, 96%, 97%, 98% or 99% identity to a CDR-L3 selected from any one of SEQ ID NOs: 390 and 415, the CDR-L2 has at least about 80%, 90%, 91%, 92%, 93%, 94%, 95%, 96%, 97%, 98% or 99% identity to a CDR-L2 selected from any one of SEQ ID NOs: 354 and 367, and the CDR-L1 has at least about 80%, 90%, 91%, 92%, 93%, 94%, 95%, 96%, 97%, 98% or 99% identity to a CDR-L1 selected from any one of SEQ ID NOs: 280, 305, and 329. In some embodiments, the CDR-H3 is a CDR-H3 selected from any one of SEQ ID NOs: 196, 218, and 250, with up to 1, 2, 3, 4, 5, 6, 7, or 8 amino acid substitutions; the CDR-H2 is a CDR-H2 selected from any one of SEQ ID NOs: 103, 136, and 169, with up to 1, 2, 3, 4, 5, 6, 7, or 8 amino acid substitutions; the CDR-H1 is a CDR-H1 selected from any one of SEQ ID NOs: 5, 840, and 48, with up to 1, 2, 3, 4, or 5 amino acid substitutions; the CDR-L3 is a CDR-L3 selected from any one of SEQ ID NOs: 390 and 415, with up to 1, 2, 3, 4, or 5 amino acid substitutions; the CDR-L2 is a CDR-L2 selected from any one of SEQ ID NOs: 354 and 367, with up to 1, 2, 3, or 4 amino acid substitutions; and the CDR-L1 is a CDR-L1 selected from any one of SEQ ID NOs: 280, 305, and 329, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 103, 136, and 169; a CDR-H3 comprising an amino acid sequence set forth in any one of SEQ ID NOs: 196, 218, and 250; a CDR-L1 comprising an amino acid sequence set forth in any one of SEQ ID NOs: 280, 305, and 329; a CDR-L2 comprising an amino acid sequence set forth in any one of SEQ ID NOs: 354 and 367; and a CDR-L3 comprising an amino acid sequence set forth in any one of SEQ ID NOs: 390 and 415.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 197, 208, and 251, the CDR-H2 has at least about 80%, 90%, 91%, 92%, 93%, 94%, 95%, 96%, 97%, 98% or 99% identity to a CDR-H2 selected from any one of SEQ ID NOs: 104, 137, and 170, the CDR-H1 has at least about 80%, 90%, 91%, 92%, 93%, 94%, 95%, 96%, 97%, 98% or 99% identity to a CDR-H1 selected from any one of SEQ ID NOs: 5, 840, and 48, the CDR-L3 has at least about 80%, 90%, 91%, 92%, 93%, 94%, 95%, 96%, 97%, 98% or 99% identity to a CDR-L3 selected from any one of SEQ ID NOs: 387 and 412, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 281, 306, and 330. In some embodiments, the CDR-H3 is a CDR-H3 selected from any one of SEQ ID NOs: 197, 208, and 251, with up to 1, 2, 3, 4, 5, 6, 7, or 8 amino acid substitutions; the CDR-H2 is a CDR-H2 selected from any one of SEQ ID NOs: 104, 137, and 170, with up to 1, 2, 3, 4, 5, 6, 7, or 8 amino acid substitutions; the CDR-H1 is a CDR-H1 selected from any one of SEQ ID NOs: 5, 840, and 48, with up to 1, 2, 3, 4, or 5 amino acid substitutions; the CDR-L3 is a CDR-L3 selected from any one of SEQ ID NOs: 387 and 412, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 281, 306, and 330, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 104, 137, and 170; a CDR-H3 comprising an amino acid sequence set forth in any one of SEQ ID NOs: 197, 208, and 251; a CDR-L1 comprising an amino acid sequence set forth in any one of SEQ ID NOs: 281, 306, and 330; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 197, 208, and 251, the CDR-H2 has at least about 80%, 90%, 91%, 92%, 93%, 94%, 95%, 96%, 97%, 98% or 99% identity to a CDR-H2 selected from any one of SEQ ID NOs: 105, 138, and 171, the CDR-H1 has at least about 80%, 90%, 91%, 92%, 93%, 94%, 95%, 96%, 97%, 98% or 99% identity to a CDR-H1 selected from any one of SEQ ID NOs: 5, 840, and 48, the CDR-L3 has at least about 80%, 90%, 91%, 92%, 93%, 94%, 95%, 96%, 97%, 98% or 99% identity to a CDR-L3 selected from any one of SEQ ID NOs: 387 and 412, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 281, 306, and 330. In some embodiments, the CDR-H3 is a CDR-H3 selected from any one of SEQ ID NOs: 197, 208, and 251, with up to 1, 2, 3, 4, 5, 6, 7, or 8 amino acid substitutions; the CDR-H2 is a CDR-H2 selected from any one of SEQ ID NOs: 105, 138, and 171, with up to 1, 2, 3, 4, 5, 6, 7, or 8 amino acid substitutions; the CDR-H1 is a CDR-H1 selected from any one of SEQ ID NOs: 5, 840, and 48, with up to 1, 2, 3, 4, or 5 amino acid substitutions; the CDR-L3 is a CDR-L3 selected from any one of SEQ ID NOs: 387 and 412, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 281, 306, and 330, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 105, 138, and 171; a CDR-H3 comprising an amino acid sequence set forth in any one of SEQ ID NOs: 197, 208, and 251; a CDR-L1 comprising an amino acid sequence set forth in any one of SEQ ID NOs: 281, 306, and 330; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 198, 219, and 252, the CDR-H2 has at least about 80%, 90%, 91%, 92%, 93%, 94%, 95%, 96%, 97%, 98% or 99% identity to a CDR-H2 selected from any one of SEQ ID NOs: 94, 131, and 162, the CDR-H1 has at least about 80%, 90%, 91%, 92%, 93%, 94%, 95%, 96%, 97%, 98% or 99% identity to a CDR-H1 selected from any one of SEQ ID NOs: 5, 840, and 48, the CDR-L3 has at least about 80%, 90%, 91%, 92%, 93%, 94%, 95%, 96%, 97%, 98% or 99% identity to a CDR-L3 selected from any one of SEQ ID NOs: 391 and 416, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 282, 307, and 331. In some embodiments, the CDR-H3 is a CDR-H3 selected from any one of SEQ ID NOs: 198, 219, and 252, with up to 1, 2, 3, 4, 5, 6, 7, or 8 amino acid substitutions; the CDR-H2 is a CDR-H2 selected from any one of SEQ ID NOs: 94, 131, and 162, with up to 1, 2, 3, 4, 5, 6, 7, or 8 amino acid substitutions; the CDR-H1 is a CDR-H1 selected from any one of SEQ ID NOs: 5, 840, and 48, with up to 1, 2, 3, 4, or 5 amino acid substitutions; the CDR-L3 is a CDR-L3 selected from any one of SEQ ID NOs: 391 and 416, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 282, 307, and 331, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 94, 131, and 162; a CDR-H3 comprising an amino acid sequence set forth in any one of SEQ ID NOs: 198, 219, and 252; a CDR-L1 comprising an amino acid sequence set forth in any one of SEQ ID NOs: 282, 307, and 331; a CDR-L2 comprising an amino acid sequence set forth in any one of SEQ ID NOs: 337 and 361; and a CDR-L3 comprising an amino acid sequence set forth in any one of SEQ ID NOs: 391 and 416.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 195, 218, and 253, the CDR-H2 has at least about 80%, 90%, 91%, 92%, 93%, 94%, 95%, 96%, 97%, 98% or 99% identity to a CDR-H2 selected from any one of SEQ ID NOs: 94, 131, and 162, the CDR-H1 has at least about 80%, 90%, 91%, 92%, 93%, 94%, 95%, 96%, 97%, 98% or 99% identity to a CDR-H1 selected from any one of SEQ ID NOs: 20, 840, and 48, the CDR-L3 has at least about 80%, 90%, 91%, 92%, 93%, 94%, 95%, 96%, 97%, 98% or 99% identity to a CDR-L3 selected from any one of SEQ ID NOs: 387 and 412, the CDR-L2 has at least about 80%, 90%, 91%, 92%, 93%, 94%, 95%, 96%, 97%, 98% or 99% identity to a CDR-L2 selected from any one of SEQ ID NOs: 355 and 367, and the CDR-L1 has at least about 80%, 90%, 91%, 92%, 93%, 94%, 95%, 96%, 97%, 98% or 99% identity to a CDR-L1 selected from any one of SEQ ID NOs: 280, 305, and 329. In some embodiments, the CDR-H3 is a CDR-H3 selected from any one of SEQ ID NOs: 195, 218, and 253, with up to 1, 2, 3, 4, 5, 6, 7, or 8 amino acid substitutions; the CDR-H2 is a CDR-H2 selected from any one of SEQ ID NOs: 94, 131, and 162, with up to 1, 2, 3, 4, 5, 6, 7, or 8 amino acid substitutions; the CDR-H1 is a CDR-H1 selected from any one of SEQ ID NOs: 20, 840, and 48, with up to 1, 2, 3, 4, or 5 amino acid substitutions; the CDR-L3 is a CDR-L3 selected from any one of SEQ ID NOs: 387 and 412, with up to 1, 2, 3, 4, or 5 amino acid substitutions; the CDR-L2 is a CDR-L2 selected from any one of SEQ ID NOs: 355 and 367, with up to 1, 2, 3, or 4 amino acid substitutions; and the CDR-L1 is a CDR-L1 selected from any one of SEQ ID NOs: 280, 305, and 329, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 20, 840, and 48; a CDR-H2 comprising an amino acid sequence set forth in any one of SEQ ID NOs: 94, 131, and 162; a CDR-H3 comprising an amino acid sequence set forth in any one of SEQ ID NOs: 195, 218, and 253; a CDR-L1 comprising an amino acid sequence set forth in any one of SEQ ID NOs: 280, 305, and 329; a CDR-L2 comprising an amino acid sequence set forth in any one of SEQ ID NOs: 355 and 367; and a CDR-L3 comprising an amino acid sequence set forth in any one of SEQ ID NOs: 387 and 412.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 180, 208, and 248, the CDR-H2 has at least about 80%, 90%, 91%, 92%, 93%, 94%, 95%, 96%, 97%, 98% or 99% identity to a CDR-H2 selected from any one of SEQ ID NOs: 94, 131, and 162, the CDR-H1 has at least about 80%, 90%, 91%, 92%, 93%, 94%, 95%, 96%, 97%, 98% or 99% identity to a CDR-H1 selected from any one of SEQ ID NOs: 5, 840, and 48, the CDR-L3 has at least about 80%, 90%, 91%, 92%, 93%, 94%, 95%, 96%, 97%, 98% or 99% identity to a CDR-L3 selected from any one of SEQ ID NOs: 387 and 412, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 280, 305, and 329. In some embodiments, the CDR-H3 is a CDR-H3 selected from any one of SEQ ID NOs: 180, 208, and 248, with up to 1, 2, 3, 4, 5, 6, 7, or 8 amino acid substitutions; the CDR-H2 is a CDR-H2 selected from any one of SEQ ID NOs: 94, 131, and 162, with up to 1, 2, 3, 4, 5, 6, 7, or 8 amino acid substitutions; the CDR-H1 is a CDR-H1 selected from any one of SEQ ID NOs: 5, 840, and 48, with up to 1, 2, 3, 4, or 5 amino acid substitutions; the CDR-L3 is a CDR-L3 selected from any one of SEQ ID NOs: 387 and 412, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 280, 305, and 329, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 5, 840, or 48; a CDR-H2 comprising an amino acid sequence set forth in any one of SEQ ID NOs: 94, 131, and 162; a CDR-H3 comprising an amino acid sequence set forth in any one of SEQ ID NOs: 180, 208, and 248; a CDR-L1 comprising an amino acid sequence set forth in any one of SEQ ID NOs: 280, 305, and 329; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 195, 218, and 253, the CDR-H2 has at least about 80%, 90%, 91%, 92%, 93%, 94%, 95%, 96%, 97%, 98% or 99% identity to a CDR-H2 selected from any one of SEQ ID NOs: 106, 137, and 170, the CDR-H1 has at least about 80%, 90%, 91%, 92%, 93%, 94%, 95%, 96%, 97%, 98% or 99% identity to a CDR-H1 selected from any one of SEQ ID NOs: 6, 840, and 48, the CDR-L3 has at least about 80%, 90%, 91%, 92%, 93%, 94%, 95%, 96%, 97%, 98% or 99% identity to a CDR-L3 selected from any one of SEQ ID NOs: 387 and 412, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 280, 305, and 329. In some embodiments, the CDR-H3 is a CDR-H3 selected from any one of SEQ ID NOs: 195, 218, and 253, with up to 1, 2, 3, 4, 5, 6, 7, or 8 amino acid substitutions; the CDR-H2 is a CDR-H2 selected from any one of SEQ ID NOs: 106, 137, and 170, with up to 1, 2, 3, 4, 5, 6, 7, or 8 amino acid substitutions; the CDR-H1 is a CDR-H1 selected from any one of SEQ ID NOs: 6, 840, and 48, with up to 1, 2, 3, 4, or 5 amino acid substitutions; the CDR-L3 is a CDR-L3 selected from any one of SEQ ID NOs: 387 and 412, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 280, 305, and 329, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 6, 840, and 48; a CDR-H2 comprising an amino acid sequence set forth in any one of SEQ ID NOs: 106, 137, and 170; a CDR-H3 comprising an amino acid sequence set forth in any one of SEQ ID NOs: 195, 218, and 253; a CDR-L1 comprising an amino acid sequence set forth in any one of SEQ ID NOs: 280, 305, and 329; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 180, 208, and 248, the CDR-H2 has at least about 80%, 90%, 91%, 92%, 93%, 94%, 95%, 96%, 97%, 98% or 99% identity to a CDR-H2 selected from any one of SEQ ID NOs: 107, 133, and 167, the CDR-H1 has at least about 80%, 90%, 91%, 92%, 93%, 94%, 95%, 96%, 97%, 98% or 99% identity to a CDR-H1 selected from any one of SEQ ID NOs: 20, 41, and 67, the CDR-L3 has at least about 80%, 90%, 91%, 92%, 93%, 94%, 95%, 96%, 97%, 98% or 99% identity to a CDR-L3 selected from any one of SEQ ID NOs: 387 and 412, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 280, 305, and 329. In some embodiments, the CDR-H3 is a CDR-H3 selected from any one of SEQ ID NOs: 180, 208, or 248, with up to 1, 2, 3, 4, 5, 6, 7, or 8 amino acid substitutions; the CDR-H2 is a CDR-H2 selected from any one of SEQ ID NOs: 107, 133, and 167, with up to 1, 2, 3, 4, 5, 6, 7, or 8 amino acid substitutions; the CDR-H1 is a CDR-H1 selected from any one of SEQ ID NOs: 20, 41, and 67, with up to 1, 2, 3, 4, or 5 amino acid substitutions; the CDR-L3 is a CDR-L3 selected from any one of SEQ ID NOs: 387 and 412, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 280, 305, and 329, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 20, 41, and 67; a CDR-H2 comprising an amino acid sequence set forth in any one of SEQ ID NOs: 107, 133, and 167; a CDR-H3 comprising an amino acid sequence set forth in any one of SEQ ID NOs: 180, 208, and 248; a CDR-L1 comprising an amino acid sequence set forth in any one of SEQ ID NOs: 280, 305, and 329; a CDR-L2 comprising an amino acid sequence set forth in any one of SEQ ID NOs: 337 and 361; and a CDR-L3 comprising an amino acid sequence set forth in any one of SEQ ID NOs: 387 and 412.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 181, 209, and 229, the CDR-H2 has at least about 80%, 90%, 91%, 92%, 93%, 94%, 95%, 96%, 97%, 98% or 99% identity to a CDR-H2 selected from any one of SEQ ID NOs: 108, 139, and 172, the CDR-H1 has at least about 80%, 90%, 91%, 92%, 93%, 94%, 95%, 96%, 97%, 98% or 99% identity to a CDR-H1 selected from any one of SEQ ID NOs: 5, 840, and 48, the CDR-L3 has at least about 80%, 90%, 91%, 92%, 93%, 94%, 95%, 96%, 97%, 98% or 99% identity to a CDR-L3 selected from any one of SEQ ID NOs: 374 and 400, the CDR-L2 has at least about 80%, 90%, 91%, 92%, 93%, 94%, 95%, 96%, 97%, 98% or 99% identity to a CDR-L2 selected from any one of SEQ ID NOs: 356 and 361, and the CDR-L1 has at least about 80%, 90%, 91%, 92%, 93%, 94%, 95%, 96%, 97%, 98% or 99% identity to a CDR-L1 selected from any one of SEQ ID NOs: 283, 308, and 332. In some embodiments, the CDR-H3 is a CDR-H3 selected from any one of SEQ ID NOs: 181, 209, and 229, with up to 1, 2, 3, 4, 5, 6, 7, or 8 amino acid substitutions; the CDR-H2 is a CDR-H2 selected from any one of SEQ ID NOs: 108, 139, and 172, with up to 1, 2, 3, 4, 5, 6, 7, or 8 amino acid substitutions; the CDR-H1 is a CDR-H1 selected from any one of SEQ ID NOs: 5, 840, and 48, with up to 1, 2, 3, 4, or 5 amino acid substitutions; the CDR-L3 is a CDR-L3 selected from any one of SEQ ID NOs: 374 and 400, with up to 1, 2, 3, 4, or 5 amino acid substitutions; the CDR-L2 is a CDR-L2 selected from any one of SEQ ID NOs: 356 and 361, with up to 1, 2, 3, or 4 amino acid substitutions; and the CDR-L1 is a CDR-L1 selected from any one of SEQ ID NOs: 283, 308, and 332, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 108, 139, and 172; a CDR-H3 comprising an amino acid sequence set forth in any one of SEQ ID NOs: 181, 209, and 229; a CDR-L1 comprising an amino acid sequence set forth in any one of SEQ ID NOs: 283, 308, and 332; a CDR-L2 comprising an amino acid sequence set forth in any one of SEQ ID NOs: 356 and 361; and a CDR-L3 comprising an amino acid sequence set forth in any one of SEQ ID NOs: 374 and 400.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 199, 220, and 254, the CDR-H2 has at least about 80%, 90%, 91%, 92%, 93%, 94%, 95%, 96%, 97%, 98% or 99% identity to a CDR-H2 selected from any one of SEQ ID NOs: 109, 140, and 173, the CDR-H1 has at least about 80%, 90%, 91%, 92%, 93%, 94%, 95%, 96%, 97%, 98% or 99% identity to a CDR-H1 selected from any one of SEQ ID NOs: 21, 42, and 68, the CDR-L3 has at least about 80%, 90%, 91%, 92%, 93%, 94%, 95%, 96%, 97%, 98% or 99% identity to a CDR-L3 selected from any one of SEQ ID NOs: 374 and 400, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 280, 305, and 329. In some embodiments, the CDR-H3 is a CDR-H3 selected from any one of SEQ ID NOs: 199, 220, and 254, with up to 1, 2, 3, 4, 5, 6, 7, or 8 amino acid substitutions; the CDR-H2 is a CDR-H2 selected from any one of SEQ ID NOs: 109, 140, and 173, with up to 1, 2, 3, 4, 5, 6, 7, or 8 amino acid substitutions; the CDR-H1 is a CDR-H1 selected from any one of SEQ ID NOs: 21, 42, and 68, with up to 1, 2, 3, 4, or 5 amino acid substitutions; the CDR-L3 is a CDR-L3 selected from any one of SEQ ID NOs: 374 and 400, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 280, 305, and 329, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 21, 42, and 68; a CDR-H2 comprising an amino acid sequence set forth in any one of SEQ ID NOs: 109, 140, and 173; a CDR-H3 comprising an amino acid sequence set forth in any one of SEQ ID NOs: 199, 220, and 254; a CDR-L1 comprising an amino acid sequence set forth in any one of SEQ ID NOs: 280, 305, and 329; a CDR-L2 comprising an amino acid sequence set forth in any one of SEQ ID NOs: 337 and 361; and a CDR-L3 comprising an amino acid sequence set forth in any one of SEQ ID NOs: 374 and 400.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 200, 221, and 255, the CDR-H2 has at least about 80%, 90%, 91%, 92%, 93%, 94%, 95%, 96%, 97%, 98% or 99% identity to a CDR-H2 selected from any one of SEQ ID NOs: 106, 137, and 170, the CDR-H1 has at least about 80%, 90%, 91%, 92%, 93%, 94%, 95%, 96%, 97%, 98% or 99% identity to a CDR-H1 selected from any one of SEQ ID NOs: 5, 43, and 69, the CDR-L3 has at least about 80%, 90%, 91%, 92%, 93%, 94%, 95%, 96%, 97%, 98% or 99% identity to a CDR-L3 selected from any one of SEQ ID NOs: 374 and 400, the CDR-L2 has at least about 80%, 90%, 91%, 92%, 93%, 94%, 95%, 96%, 97%, 98% or 99% identity to a CDR-L2 selected from any one of SEQ ID NOs: 337 and 361, and the CDR-L1 has at least about 80%, 90%, 91%, 92%, 93%, 94%, 95%, 96%, 97%, 98% or 99% identity to a CDR-L1 selected from any one of SEQ ID NOs: 284, 309, and 333. In some embodiments, the CDR-H3 is a CDR-H3 selected from any one of SEQ ID NOs: 200, 221, and 255, with up to 1, 2, 3, 4, 5, 6, 7, or 8 amino acid substitutions; the CDR-H2 is a CDR-H2 selected from any one of SEQ ID NOs: 106, 137, and 170, with up to 1, 2, 3, 4, 5, 6, 7, or 8 amino acid substitutions; the CDR-H1 is a CDR-H1 selected from any one of SEQ ID NOs: 5, 43, and 69, with up to 1, 2, 3, 4, or 5 amino acid substitutions; the CDR-L3 is a CDR-L3 selected from any one of SEQ ID NOs: 374 or 400, with up to 1, 2, 3, 4, or 5 amino acid substitutions; the CDR-L2 is a CDR-L2 selected from any one of SEQ ID NOs: 337 and 361, with up to 1, 2, 3, or 4 amino acid substitutions; and the CDR-L1 is a CDR-L1 selected from any one of SEQ ID NOs: 284, 309, and 333, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 5, 43, and 69; a CDR-H2 comprising an amino acid sequence set forth in any one of SEQ ID NOs: 106, 137, and 170; a CDR-H3 comprising an amino acid sequence set forth in any one of SEQ ID NOs: 200, 221, and 255; a CDR-L1 comprising an amino acid sequence set forth in any one of SEQ ID NOs: 284, 309, and 333; a CDR-L2 comprising an amino acid sequence set forth in any one of SEQ ID NOs: 337 and 361; and a CDR-L3 comprising an amino acid sequence set forth in any one of SEQ ID NOs: 374 and 400.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 201, 222, and 256, the CDR-H2 has at least about 80%, 90%, 91%, 92%, 93%, 94%, 95%, 96%, 97%, 98% or 99% identity to a CDR-H2 selected from any one of SEQ ID NOs: 94, 131, and 162, the CDR-H1 has at least about 80%, 90%, 91%, 92%, 93%, 94%, 95%, 96%, 97%, 98% or 99% identity to a CDR-H1 selected from any one of SEQ ID NOs: 5, 43, and 69, the CDR-L3 has at least about 80%, 90%, 91%, 92%, 93%, 94%, 95%, 96%, 97%, 98% or 99% identity to a CDR-L3 selected from any one of SEQ ID NOs: 392 and 417, the CDR-L2 has at least about 80%, 90%, 91%, 92%, 93%, 94%, 95%, 96%, 97%, 98% or 99% identity to a CDR-L2 selected from any one of SEQ ID NOs: 357 or 361, and the CDR-L1 has at least about 80%, 90%, 91%, 92%, 93%, 94%, 95%, 96%, 97%, 98% or 99% identity to a CDR-L1 selected from any one of SEQ ID NOs: 285, 310, and 334. In some embodiments, the CDR-H3 is a CDR-H3 selected from any one of SEQ ID NOs: 201, 222, or 256, with up to 1, 2, 3, 4, 5, 6, 7, or 8 amino acid substitutions; the CDR-H2 is a CDR-H2 selected from any one of SEQ ID NOs: 94, 131, and 162, with up to 1, 2, 3, 4, 5, 6, 7, or 8 amino acid substitutions; the CDR-H1 is a CDR-H1 selected from any one of SEQ ID NOs: 5, 43, and 69, with up to 1, 2, 3, 4, or 5 amino acid substitutions; the CDR-L3 is a CDR-L3 selected from any one of SEQ ID NOs: 392 and 417, with up to 1, 2, 3, 4, or 5 amino acid substitutions; the CDR-L2 is a CDR-L2 selected from any one of SEQ ID NOs: 357 and 361, with up to 1, 2, 3, or 4 amino acid substitutions; and the CDR-L1 is a CDR-L1 selected from any one of SEQ ID NOs: 285, 310, and 334, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 5, 43, and 69; a CDR-H2 comprising an amino acid sequence set forth in any one of SEQ ID NOs: 94, 131, and 162; a CDR-H3 comprising an amino acid sequence set forth in any one of SEQ ID NOs: 201, 222, and 256; a CDR-L1 comprising an amino acid sequence set forth in any one of SEQ ID NOs: 285, 310, and 334; a CDR-L2 comprising an amino acid sequence set forth in any one of SEQ ID NOs: 357 and 361; and a CDR-L3 comprising an amino acid sequence set forth in any one of SEQ ID NOs: 392 and 417.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 202, 222, and 257, the CDR-H2 has at least about 80%, 90%, 91%, 92%, 93%, 94%, 95%, 96%, 97%, 98% or 99% identity to a CDR-H2 selected from any one of SEQ ID NOs: 110, 141, and 174, the CDR-H1 has at least about 80%, 90%, 91%, 92%, 93%, 94%, 95%, 96%, 97%, 98% or 99% identity to a CDR-H1 selected from any one of SEQ ID NOs: 5, 43, and 69, the CDR-L3 has at least about 80%, 90%, 91%, 92%, 93%, 94%, 95%, 96%, 97%, 98% or 99% identity to a CDR-L3 selected from any one of SEQ ID NOs: 393 and 418, the CDR-L2 has at least about 80%, 90%, 91%, 92%, 93%, 94%, 95%, 96%, 97%, 98% or 99% identity to a CDR-L2 selected from any one of SEQ ID NOs: 358 and 361, and the CDR-L1 has at least about 80%, 90%, 91%, 92%, 93%, 94%, 95%, 96%, 97%, 98% or 99% identity to a CDR-L1 selected from any one of SEQ ID NOs: 285, 310, and 334. In some embodiments, the CDR-H3 is a CDR-H3 selected from any one of SEQ ID NOs: 202, 222, and 257, with up to 1, 2, 3, 4, 5, 6, 7, or 8 amino acid substitutions; the CDR-H2 is a CDR-H2 selected from any one of SEQ ID NOs: 110, 141, or 174, with up to 1, 2, 3, 4, 5, 6, 7, or 8 amino acid substitutions; the CDR-H1 is a CDR-H1 selected from any one of SEQ ID NOs: 5, 43, and 69, with up to 1, 2, 3, 4, or 5 amino acid substitutions; the CDR-L3 is a CDR-L3 selected from any one of SEQ ID NOs: 393 or 418, with up to 1, 2, 3, 4, or 5 amino acid substitutions; the CDR-L2 is a CDR-L2 selected from any one of SEQ ID NOs: 358 and 361, with up to 1, 2, 3, or 4 amino acid substitutions; and the CDR-L1 is a CDR-L1 selected from any one of SEQ ID NOs: 285, 310, and 334, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 5, 43, and 69; a CDR-H2 comprising an amino acid sequence set forth in any one of SEQ ID NOs: 110, 141, and 174; a CDR-H3 comprising an amino acid sequence set forth in any one of SEQ ID NOs: 202, 222, and 257; a CDR-L1 comprising an amino acid sequence set forth in any one of SEQ ID NOs: 285, 310, and 334; a CDR-L2 comprising an amino acid sequence set forth in any one of SEQ ID NOs: 358 and 361; and a CDR-L3 comprising an amino acid sequence set forth in any one of SEQ ID NOs: 393 and 418.

In some embodiments, an antibody, or antigen binding fragment thereof, provided herein comprises a CDR-H3 comprising an amino acid sequence set forth in any one of SEQ ID NOs: 545, 546, and 547; and a CDR-L3 comprising an amino acid sequence set forth in any one of SEQ ID NOs: 548, 549, and 550.

In some embodiments, an antibody, or antigen binding fragment thereof, provided herein comprises a CDR-H1 comprising an amino acid sequence set forth in any one of SEQ ID NOs: 551, 557, and 563; a CDR-H2 comprising an amino acid sequence set forth in any one of SEQ ID NOs: 552, 558, and 564; a CDR-H3 comprising an amino acid sequence set forth in any one of SEQ ID NOs: 553, 559, and 565; a CDR-L1 comprising an amino acid sequence set forth in any one of SEQ ID NOs: 554, 560, and 566; a CDR-L2 comprising an amino acid sequence set forth in any one of SEQ ID NOs: 555, 561, and 567; and a CDR-L3 comprising an amino acid sequence set forth in any one of SEQ ID NOs: 556, 562, and 568.

In some embodiments, an antibody, or antigen binding fragment thereof, provided herein comprises a CDR-H1 comprising an amino acid sequence set forth in any one of SEQ ID NOs: 569, 575, and 581; a CDR-H2 comprising an amino acid sequence set forth in any one of SEQ ID NOs: 570, 576, and 582; a CDR-H3 comprising an amino acid sequence set forth in any one of SEQ ID NOs: 571, 577, and 583; a CDR-L1 comprising an amino acid sequence set forth in any one of SEQ ID NOs: 572, 578, and 584; a CDR-L2 comprising an amino acid sequence set forth in any one of SEQ ID NOs: 573, 579, and 585; and a CDR-L3 comprising an amino acid sequence set forth in any one of SEQ ID NOs: 574, 580, and 586.

In some embodiments, an antibody, or antigen binding fragment thereof, provided herein comprises a CDR-H1 comprising an amino acid sequence set forth in any one of SEQ ID NOs: 587, 592, and 596; a CDR-H2 comprising an amino acid sequence set forth in any one of SEQ ID NOs: 588, 593, and 597; a CDR-H3 comprising an amino acid sequence set forth in any one of SEQ ID NOs: 589, 207, and 598; a CDR-L1 comprising an amino acid sequence set forth in any one of SEQ ID NOs: 590, 594, and 599; a CDR-L2 comprising an amino acid sequence set forth in any one of SEQ ID NOs: 338, 362, and 362; and a CDR-L3 comprising an amino acid sequence set forth in any one of SEQ ID NOs: 591, 595, and 600.

In some embodiments, an antibody, or antigen binding fragment thereof, provided herein comprises a CDR-H1 comprising an amino acid sequence set forth in any one of SEQ ID NOs: 601, 607, and 613; a CDR-H2 comprising an amino acid sequence set forth in any one of SEQ ID NOs: 602, 608, and 614; a CDR-H3 comprising an amino acid sequence set forth in any one of SEQ ID NOs: 603, 609, and 615; a CDR-L1 comprising an amino acid sequence set forth in any one of SEQ ID NOs: 604, 610, and 616; a CDR-L2 comprising an amino acid sequence set forth in any one of SEQ ID NOs: 605, 611, and 617; and a CDR-L3 comprising an amino acid sequence set forth in any one of SEQ ID NOs: 606, 612, and 618.

In some embodiments, an antibody, or antigen binding fragment thereof, provided herein comprises a CDR-H1 comprising an amino acid sequence set forth in any one of SEQ ID NOs: 619, 626, and 631; a CDR-H2 comprising an amino acid sequence set forth in any one of SEQ ID NOs: 620, 626, and 632; a CDR-H3 comprising an amino acid sequence set forth in any one of SEQ ID NOs: 621, 627, and 633; a CDR-L1 comprising an amino acid sequence set forth in any one of SEQ ID NOs: 622, 628, and 634; a CDR-L2 comprising an amino acid sequence set forth in any one of SEQ ID NOs: 623, 629, and 635; and a CDR-L3 comprising an amino acid sequence set forth in any one of SEQ ID NOs: 624, 630, and 636.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 763, 769, and 775, the CDR-H2 has at least about 80%, 90%, 91%, 92%, 93%, 94%, 95%, 96%, 97%, 98% or 99% identity to a CDR-H2 selected from any one of SEQ ID NOs: 748, 116, and 758, the CDR-H1 has at least about 80%, 90%, 91%, 92%, 93%, 94%, 95%, 96%, 97%, 98% or 99% identity to a CDR-H1 selected from any one of SEQ ID NOs: 5, 840, and 48, the CDR-L3 has at least about 80%, 90%, 91%, 92%, 93%, 94%, 95%, 96%, 97%, 98% or 99% identity to a CDR-L3 selected from any one of SEQ ID NOs: 806 and 812, the CDR-L2 has at least about 80%, 90%, 91%, 92%, 93%, 94%, 95%, 96%, 97%, 98% or 99% identity to a CDR-L2 selected from any one of SEQ ID NOs: 797, and 802, and the CDR-L1 has at least about 80%, 90%, 91%, 92%, 93%, 94%, 95%, 96%, 97%, 98% or 99% identity to a CDR-L1 selected from any one of SEQ ID NOs: 781, 787, and 792. In some embodiments, the CDR-H3 is a CDR-H3 selected from any one of SEQ ID NOs: 763, 769, and 775, with up to 1, 2, 3, 4, 5, 6, 7, or 8 amino acid substitutions; the CDR-H2 is a CDR-H2 selected from any one of SEQ ID NOs: 748, 116, and 758, with up to 1, 2, 3, 4, 5, 6, 7, or 8 amino acid substitutions; the CDR-H1 is a CDR-H1 selected from any one of SEQ ID NOs: 5, 840, and 48, with up to 1, 2, 3, 4, or 5 amino acid substitutions; the CDR-L3 is a CDR-L3 selected from any one of SEQ ID NOs: 806 and 812, with up to 1, 2, 3, 4, or 5 amino acid substitutions; the CDR-L2 is a CDR-L2 selected from any one of SEQ ID NOs: 797 and 802, with up to 1, 2, 3, or 4 amino acid substitutions; and the CDR-L1 is a CDR-L1 selected from any one of SEQ ID NOs: 781, 787, and 792, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 748, 116, and 758; a CDR-H3 comprising an amino acid sequence set forth in any one of SEQ ID NOs: 763, 769, and 775, a CDR-L1 comprising an amino acid sequence set forth in any one of SEQ ID NOs: 781, 787, and 792; a CDR-L2 comprising an amino acid sequence set forth in any one of SEQ ID NOs: 797 and 802; and a CDR-L3 comprising an amino acid sequence set forth in any one of SEQ ID NOs: 806 and 812.

In some embodiments, an antibody, or antigen binding fragment thereof, provided herein comprises a CDR-H1 comprising an amino acid sequence set forth in any one of SEQ ID NOs: 740, 742, and 745; a CDR-H2 comprising an amino acid sequence set forth in any one of SEQ ID NOs: 749, 754, and 759; a CDR-H3 comprising an amino acid sequence set forth in any one of SEQ ID NOs: 764, 770, and 776, a CDR-L1 comprising an amino acid sequence set forth in any one of SEQ ID NOs: 782, 788, and 793; a CDR-L2 comprising an amino acid sequence set forth in any one of SEQ ID NOs: 798 and 803; and a CDR-L3 comprising an amino acid sequence set forth in any one of SEQ ID NOs: 807 and 813.

In some embodiments, an antibody, or antigen binding fragment thereof, provided herein comprises a CDR-H1 comprising an amino acid sequence set forth in any one of SEQ ID NOs: 17, 743, and 746; a CDR-H2 comprising an amino acid sequence set forth in any one of SEQ ID NOs: 750, 133, and 165; a CDR-H3 comprising an amino acid sequence set forth in any one of SEQ ID NOs: 765, 771, and 777, a CDR-L1 comprising an amino acid sequence set forth in any one of SEQ ID NOs: 783, 298, and 322; a CDR-L2 comprising an amino acid sequence set forth in any one of SEQ ID NOs: 799 and 804; and a CDR-L3 comprising an amino acid sequence set forth in any one of SEQ ID NOs: 808 and 814.

In some embodiments, an antibody, or antigen binding fragment thereof, provided herein comprises a CDR-H1 comprising an amino acid sequence set forth in any one of SEQ ID NOs: 5, 840, and 48; a CDR-H2 comprising an amino acid sequence set forth in any one of SEQ ID NOs: 751, 755, and 760; a CDR-H3 comprising an amino acid sequence set forth in any one of SEQ ID NOs: 766, 772, and 778, a CDR-L1 comprising an amino acid sequence set forth in any one of SEQ ID NOs: 784, 789, and 794; a CDR-L2 comprising an amino acid sequence set forth in any one of SEQ ID NOs: 341 and 361; and a CDR-L3 comprising an amino acid sequence set forth in any one of SEQ ID NOs: 809 and 815.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 223, a CDR-L1 comprising an amino acid sequence set forth in any one of SEQ ID NOs: 260, 288, and 313; a CDR-L2 comprising an amino acid sequence set forth in any one of SEQ ID NOs: 341 and 361; and a CDR-L3 comprising an amino acid sequence set forth in any one of SEQ ID NOs: 368 and 394.

In some embodiments, an antibody, or antigen binding fragment thereof, provided herein comprises a CDR-H1 comprising an amino acid sequence set forth in any one of SEQ ID NOs: 8, 26, and 51; a CDR-H2 comprising an amino acid sequence set forth in any one of SEQ ID NOs: 752, 756, and 761; a CDR-H3 comprising an amino acid sequence set forth in any one of SEQ ID NOs: 767, 773, and 779, a CDR-L1 comprising an amino acid sequence set forth in any one of SEQ ID NOs: 785, 790, and 795; a CDR-L2 comprising an amino acid sequence set forth in any one of SEQ ID NOs: 800 and 805; and a CDR-L3 comprising an amino acid sequence set forth in any one of SEQ ID NOs: 810 and 816.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 767, 773, and 779, the CDR-H2 has at least about 80%, 90%, 91%, 92%, 93%, 94%, 95%, 96%, 97%, 98% or 99% identity to a CDR-H2 selected from any one of SEQ ID NOs: 752, 756, and 761, the CDR-H1 has at least about 80%, 90%, 91%, 92%, 93%, 94%, 95%, 96%, 97%, 98% or 99% identity to a CDR-H1 selected from any one of SEQ ID NOs: 8, 26, and 51, the CDR-L3 has at least about 80%, 90%, 91%, 92%, 93%, 94%, 95%, 96%, 97%, 98% or 99% identity to a CDR-L3 selected from any one of SEQ ID NOs: 810 and 816, the CDR-L2 has at least about 80%, 90%, 91%, 92%, 93%, 94%, 95%, 96%, 97%, 98% or 99% identity to a CDR-L2 selected from any one of SEQ ID NOs: 800 and 805, and the CDR-L1 has at least about 80%, 90%, 91%, 92%, 93%, 94%, 95%, 96%, 97%, 98% or 99% identity to a CDR-L1 selected from any one of SEQ ID NOs: 785, 790, and 795. In some embodiments, the CDR-H3 is a CDR-H3 selected from any one of SEQ ID NOs: 767, 773, and 779, with up to 1, 2, 3, 4, 5, 6, 7, or 8 amino acid substitutions; the CDR-H2 is a CDR-H2 selected from any one of SEQ ID NOs: 752, 756, and 761, with up to 1, 2, 3, 4, 5, 6, 7, or 8 amino acid substitutions; the CDR-H1 is a CDR-H1 selected from any one of SEQ ID NOs: 8, 26, and 51, with up to 1, 2, 3, 4, or 5 amino acid substitutions; the CDR-L3 is a CDR-L3 selected from any one of SEQ ID NOs: 810 and 816, with up to 1, 2, 3, 4, or 5 amino acid substitutions; the CDR-L2 is a CDR-L2 selected from any one of SEQ ID NOs: 800 and 805, with up to 1, 2, 3, or 4 amino acid substitutions; and the CDR-L1 is a CDR-L1 selected from any one of SEQ ID NOs: 785, 790, and 795, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 741, 744, and 747; a CDR-H2 comprising an amino acid sequence set forth in any one of SEQ ID NOs: 753, 757, and 762; a CDR-H3 comprising an amino acid sequence set forth in any one of SEQ ID NOs: 768, 774, and 780, a CDR-L1 comprising an amino acid sequence set forth in any one of SEQ ID NOs: 786, 791, and 796; a CDR-L2 comprising an amino acid sequence set forth in any one of SEQ ID NOs: 801 and 360; and a CDR-L3 comprising an amino acid sequence set forth in any one of SEQ ID NOs: 811 and 817.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 768, 774, and 780, the CDR-H2 has at least about 80%, 90%, 91%, 92%, 93%, 94%, 95%, 96%, 97%, 98% or 99% identity to a CDR-H2 selected from any one of SEQ ID NOs: 753, 757, and 762, the CDR-H1 has at least about 80%, 90%, 91%, 92%, 93%, 94%, 95%, 96%, 97%, 98% or 99% identity to a CDR-H1 selected from any one of SEQ ID NOs: 741, 744, and 747, the CDR-L3 has at least about 80%, 90%, 91%, 92%, 93%, 94%, 95%, 96%, 97%, 98% or 99% identity to a CDR-L3 selected from any one of SEQ ID NOs: 811 and 817, the CDR-L2 has at least about 80%, 90%, 91%, 92%, 93%, 94%, 95%, 96%, 97%, 98% or 99% identity to a CDR-L2 selected from any one of SEQ ID NOs: 801 and 360, and the CDR-L1 has at least about 80%, 90%, 91%, 92%, 93%, 94%, 95%, 96%, 97%, 98% or 99% identity to a CDR-L1 selected from any one of SEQ ID NOs: 786, 791, and 796. In some embodiments, the CDR-H3 is a CDR-H3 selected from any one of SEQ ID NOs: 768, 774, and 780, with up to 1, 2, 3, 4, 5, 6, 7, or 8 amino acid substitutions; the CDR-H2 is a CDR-H2 selected from any one of SEQ ID NOs: 753, 757, and 762, with up to 1, 2, 3, 4, 5, 6, 7, or 8 amino acid substitutions; the CDR-H1 is a CDR-H1 selected from any one of SEQ ID NOs: 741, 744, and 747, with up to 1, 2, 3, 4, or 5 amino acid substitutions; the CDR-L3 is a CDR-L3 selected from any one of SEQ ID NOs: 811 and 817, with up to 1, 2, 3, 4, or 5 amino acid substitutions; the CDR-L2 is a CDR-L2 selected from any one of SEQ ID NOs: 801 and 360, with up to 1, 2, 3, or 4 amino acid substitutions; and the CDR-L1 is a CDR-L1 selected from any one of SEQ ID NOs: 786, 791, and 796, with up to 1, 2, 3, 4, 5, or 6 amino acid substitutions.

In some embodiments, an antibody, or antigen binding fragment thereof, provided herein comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 234, a CDR-L1 comprising an amino acid sequence set forth in any one of SEQ ID NOs: 260, 288, and 313; a CDR-L2 comprising an amino acid sequence set forth in any one of SEQ ID NOs: 347 and 365; and a CDR-L3 comprising an amino acid sequence set forth in any one of SEQ ID NOs: 378 and 404.

In some embodiments, the CDR-H3 has at least about 80%, 90%, 91%, 92%, 93%, 94%, 95%, 96%, 97%, 98% or 99% identity to a CDR-H3 selected from any one of SEQ ID NOs: 175, 203, and 234, the CDR-H2 has at least about 80%, 90%, 91%, 92%, 93%, 94%, 95%, 96%, 97%, 98% or 99% identity to a CDR-H2 selected from any one of SEQ ID NOs: 70, 111, and 142, the CDR-H1 has at least about 80%, 90%, 91%, 92%, 93%, 94%, 95%, 96%, 97%, 98% or 99% identity to a CDR-H1 selected from any one of SEQ ID NOs: 10, 27, and 52, the CDR-L3 has at least about 80%, 90%, 91%, 92%, 93%, 94%, 95%, 96%, 97%, 98% or 99% identity to a CDR-L3 selected from any one of SEQ ID NOs: 378 and 404, the CDR-L2 has at least about 80%, 90%, 91%, 92%, 93%, 94%, 95%, 96%, 97%, 98% or 99% identity to a CDR-L2 selected from any one of SEQ ID NOs: 347 and 365, and the CDR-L1 has at least about 80%, 90%, 91%, 92%, 93%, 94%, 95%, 96%, 97%, 98% or 99% identity to a CDR-L1 selected from any one of SEQ ID NOs: 260, 288, and 313. In some embodiments, the CDR-H3 is a CDR-H3 selected from any one of SEQ ID NOs: 175, 203, and 234, with up to 1, 2, 3, 4, 5, 6, 7, or 8 amino acid substitutions; the CDR-H2 is a CDR-H2 selected from any one of SEQ ID NOs: 70, 111, and 142, with up to 1, 2, 3, 4, 5, 6, 7, or 8 amino acid substitutions; the CDR-H1 is a CDR-H1 selected from any one of SEQ ID NOs: 10, 27, and 52, with up to 1, 2, 3, 4, or 5 amino acid substitutions; the CDR-L3 is a CDR-L3 selected from any one of SEQ ID NOs: 378 and 404, with up to 1, 2, 3, 4, or 5 amino acid substitutions; the CDR-L2 is a CDR-L2 selected from any one of SEQ ID NOs: 347 and 365, with up to 1, 2, 3, or 4 amino acid substitutions; and the CDR-L1 is a CDR-L1 selected from any one of SEQ ID NOs: 260, 288, and 313, with up to 1, 2, 3, 4, 5, or 6 amino acid substitutions.

TABLE 4
Consensus sequences of clones

| Consensus Sequence | SEQ ID NO: |
|---|---|
| Variants of 327 | |
| CDRH3 (Kabat) EGYDLWSGX₁SSNWLDP, where X₁ = | 545 |
| D, E, H, or Y | |
| CDRH3 (Chothia) GYDLWSGX₁SSNWLD, where X₁ = | 546 |
| D, E, H, or Y | |
| CDRH3 (IMGT) AREGYDLWSGX₁SSNWLDP, where | 547 |
| X₁ = D, E, H, or Y | |
| CDRL3 (Kabat) QQLX₁X₂YPRT, where X₁ = N or Q, | 548 |
| and X₂ = S or N | |
| CDRL3 (Chothia) LX₁X₂YPR, where X₁ = N or Q, and | 549 |
| X₂ = S or N | |
| CDRL3 (IMGT) QQLX₁X₂YPRT, where X₁ = N or Q, | 550 |
| and X₂ = S or N | |
| Variants of 114 | |
| CDRH1 (Kabat) X₁X₂AMX₃, where X₁ = N, S, T, or I, | |
| X₂ = Y or H, and X₃ = T, Y, or S | |
| CDRH2 (Kabat) LIX₁GX₂GX₃X₄TX₅YADSVX₆G, | 552 |
| where X₁ = S or T, X₂ = S or G, X₃ = G, A, or S, X₄ = L, S, I, or | |
| V, X₅ = K, H, N, or Y, and X₆ = K or R | |
| CDRH3 (Kabat) X₁EGLTX₂GEX₃, where X₁ = D or E, | 553 |
| X₂ = T or S, and X₃ = Y, F, or S | |
| CDRL1 (Kabat) RX₁SQX₂IRNX₃LX₄, where X₁ = A or | 554 |
| T, X₂ = D, G, or A, X₃ = D or N, and X₄ = A, G, or D | |
| CDRL2 (Kabat) X₁X₂SX₃X₄X₅S, where X₁ = A or V, | |
| X₂ = S, V, T, or A, X₃ = S or N, X₄ = L or F, and X₅ = Q or K | |
| CDRL3 (Kabat) LQHX₁X₂YPFX₃, where X₁ = N or S, | 556 |
| X₂ = N, S, or T, and X₃ = T or S | |
| CDRH1 (Chothia) GFTFX₁X₂X₃, where X₁ = S, R, N, | 557 |
| G, or T, X₂ = N, S, T, or I, and X₃ = Y or H | |
| CDRH2 (Chothia) X₁GX₂GX₃X₄, where X₁ = S or T, X₂ = | |
| S or G, X₃ = G, A, or S, and X₄ = L, S, I, or V | |
| CDRH3 (Chothia) EGLTX₁GE, where X₁ = T or S | 559 |
| CDRL1 (Chothia) SQX₁IRNX₂, where X₁ = D, G, or A, | 560 |
| and X₂ = D or N | |
| CDRL2 (Chothia) X₁X₂S, where X₁ = A or V, and X₂ = | |
| S, V, T, or A | |
| CDRL3 (Chothia) HX₁X₂YPF, where X₁ = N or S, and | 562 |
| X₂ = N, S, or T | |
| CDRH1 (IMGT) GFTFX₁X₂X₃A, where X₁ = S, R, N, | 563 |
| G, or T, X₂ = N, S, T, or I, and X₃ = Y or H | |
| CDRH2 (IMGT) IX₁GX₂GX₃X₄T, where X₁ = S or T, | 564 |
| X₂ = S or G, X₃ = G, A, or S, and X₄ = L, S, I, or V | |
| CDRH3 (IMGT) X₁KX₂EGLTX₃GEX₄, where X₁ = V | 565 |
| or A, X₂ = D or E, X₃ = T or S, and X₄ = Y, F, or S | |
| CDRL1 (IMGT) QX₁IRNX₂, where X₁ = D, G, or A, | 566 |
| and X₂ = D or N | |
| CDRL2 (IMGT) X₁X₂S, where X₁ = A or V, and X₂ = | |
| S, V, T, or A | |
| CDRL3 (IMGT) LQHX₁X₂YPFX₃, where X₁ = N or S, | 568 |
| X₂ = N, S, or T, and X₃ = T or S | |
| Variants of 122 | |
| CDRH1 (Kabat) X₁X₂WMH, where X₁ = N or S, and X₂ = | |
| Y or F | |
| CDRH2 (Kabat) RIX₁X₂X₃GX₄X₅TRYADSVKG, | 570 |
| where X₁ = N, D, or S, X₂ = S or N, X₃ = D or N, X₄ = I or F, | |
| and X₅ = S or N | |
| CDRH3 (Kabat) DFQLELX₁DX₂, where X₁ = P or R, | 571 |
| and X₂ = Y or F | |
| CDRL1 (Kabat) RASQX₁IRX₂DLX₃, where X₁ = G or | 572 |
| A, X₂ = D or N, and X₃ = A or G | |
| CDRL2 (Kabat) AX₁SX₂LQX₃, where X₁ = A or T, X₂ = | 573 |
| S or I, and X₃ = S or N | |
| CDRL3 (Kabat) LQHX₁SNPFT, where X₁ = K, N, or Y | 574 |
| CDRH1 (Chothia) GX₁TFX₂X₃X₄, where X₁ = F or L, | |
| X₂ = S or R, and X₃ = N or S, and X₄ = Y or F | |
| CDRH2 (Chothia) X₁X₂X₃GX₄X₅, where X₁ = N, D, or | |
| S, X₂ = S or N, X₃ = D or N, X₄ = I or F, and X₅ = S or N | |
| CDRH3 (Chothia) FQLELX₁D, where X₁ = P or R | 577 |
| CDRL1 (Chothia) SQX₁IRX₂D, where X₁ = G or A, and | 578 |
| X₂ = D or N | |
| CDRL2 (Chothia) AX₁S, where X₁ = A or T | |
| CDRL3 (Chothia) HX₁SNPF, where X₁ = K, N, or Y | 580 |
| CDRH1 (IMGT) GX₁TFX₂X₃X₄W, where X₁ = F or L, | 581 |
| X₂ = S or R, and X₃ = N or S, and X₄ = Y or F | |
| CDRH2 (IMGT) IX₁X₂X₃GX₄X₅T, where X₁ = N, D, or | |
| S, X₂ = S or N, X₃ = D or N, X₄ = I or F, and X₅ = S or N | |
| CDRH3 (IMGT) VX₁DFQLELX₂DX₃, where X₁ = R or | 583 |
| S, X₂ = P or R, and X₃ = Y or F | |
| CDRL1 (IMGT) QX₁IRX₂D, where X₁ = G or A, X₂ = | 584 |
| D or N | |
| CDRL2 (IMGT) AX₁S, where X₁ = A or T | |
| CDRL3 (IMGT) LQHX₁SNPFT, where X₁ = K, N, or Y | 586 |
| Variants of 127 | |
| CDRH1 (Kabat) X₁AWMS, where X₁ = N or S | 587 |
| CDRH2 (Kabat) | 588 |
| RX₁KX₂X₃X₄X₅GGTX₆DX₇X₈APX₉X₁₀G, where X₁ = I or V, | |
| X₂ = S or G, X₃ = N, V, or K, X₄ = S or T, X₅ = Y, A, D, or T, | |
| X₆ = T or A, X₇ = Y or F, X₈ = A or P, X₉ = A or V, and X₁₀ = I | |
| or K | |
| CDRH3 (Kabat) DHPKFGFDX₁, where X₁ = F or Y | 589 |
| CDRL1 (Kabat) KSSQSVX₁YX₂SX₃X₄KX₅YLX₆, | 590 |
| where X₁ = F or L, X₂ = S or T, and X₃ = N or S, X₄ = N or S, | |
| X₅ = N or H, and X₆ = A or N | |
| CDRL2 (Kabat) WASTRES | 338 |
| CDRL3 (Kabat) QQX₁YX₂X₃PRT, where X₁ = H or Y, | 591 |
| X₂ = D or S, and X₃ = I or T | |
| CDRH1 (Chothia) GFTFX₁X₂A, where X₁ = S, H, or N, | 592 |
| X₂ = N or S | |
| CDRH2 (Chothia) KX₁X₂X₃X₄GGT, where X₁ = S or | 593 |
| G, X₂ = N, V, or K, X₃ = S or T, and X₄ = Y, A, D, or T | |
| CDRH3 (Chothia) HPKFGFD | 207 |
| CDRL1 (Chothia) SQSVX₁YX₂SX₃X₄KX₅Y, where X₁ = | 594 |
| F or L, X₂ = S or T, and X₃ = N or S, X₄ = N or S, and X₅ = N | |
| or H | |
| CDRL2 (Chothia) WAS | |
| CDRL3 (Chothia) X₁YX₂X₃PR, where X₁ = H or Y, X₂ = | |
| D or S, and X₃ = I or T | |
| CDRH1 (IMGT) GFTFX₁X₂AW, where X₁ = S, H, or | 596 |
| N, X₂ = N or S | |
| CDRH2 (IMGT) X₁KX₂X₃X₄X₅GGTX₆, where X₁ = I | 597 |
| or V, X₂ = S or G, X₃ = N, V, or K, X₄ = S or T, X₅ = Y, A, D, | |
| or T, X₆ = T or A | |
| CDRH3 (IMGT) TTDHPKFGFDX₁, where X₁ = F or Y | 598 |
| CDRL1 (IMGT) QSVX₁YX₂SX₃X₄KX₅Y, where X₁ = | 599 |
| For L, X₂ = S or T, and X₃ = N or S, X₄ = N or S, X₅ = N or H | |
| CDRL2 (IMGT) WAS | |
| CDRL3 (IMGT) QQX₁YX₂X₃PRT, where X₁ = H or Y, | 600 |
| X₂ = D or S, and X₃ = I or T | |
| Variants of 128 | |
| CDRH1 (Kabat) X₁AWMX₂, where X₁ = N or Y, and | |
| X₂ = T, R, N, or S | |
| CDRH2 (Kabat) | 602 |
| X₁IKX₂X₃X₄X₅GGTX₆X₇X₈X₉APVX₁₀G, where X₁ = R or H, | |
| X₂ = S, R, or T, X₃ = I, E, K, L, N, or Q, X₄ = S or T, X₅ = Y or | |
| D, X₆ = T or R, X₇ = D or E, X₈ = S or Y, X₉ = A or D, and X₁₀ = | |
| K or Q | |
| CDRH3 (Kabat)GX₁VWX₂YGGSLDX₃, where X₁ = S | 603 |
| or G, X₂ = K, N, or R, and X₃ = F or Y | |
| CDRL1 (Kabat) RASQX₁IX₂RX₃LN, where X₁ = G, S, | 604 |
| F, N, or I, X₂ = S or T, and X₃ = F, Y, or S | |
| CDRL2 (Kabat) X₁ASX₂LX₃X₄, where X₁ = A or G, X₂ = | |
| T, S, or R, X₃ = Q or E, and X₄ = N or S | |
| CDRL3 (Kabat) QQX₁YX₂X₃X₄X₅T, where X₁ = T or | 606 |
| S, X₂ = S, N, R, or G, X₃ = T or S, X₄ = P or S, and X₅ = L or V | |
| CDRH1 (Chothia) GFX₁FX₂X₃A, where X₁ = T or I, X₂ = | 607 |
| N, I, or S, and X₃ = N, or Y | |
| CDRH2 (Chothia) KX₁X₂X₃X₄GGT, where X₁ = S, R, | 608 |
| or T, X₂ = I, E, K, L, N, or Q, X₃ = S or T, and X₄ = Y or D | |
| CDRH3 (Chothia) X₁VWX₂YGGSLD, where X₁ = S or | 609 |
| G, and X₂ = K, N, or R | |
| CDRL1 (Chothia) SQX₁IX₂RX₃, where X₁ = G, S, F, N, | 610 |
| or I, X₂ = S or T, and X₃ = F, Y, or S | |
| CDRL2 (Chothia) X₁AS, where X₁ = A or G | |
| CDRL3 (Chothia) X₁YX₂X₃X₄X₅, where X₁ = T or S, | |
| X₂ = S, N, R, or G, X₃ = T or S, X₄ = P or S, and X₅ = L or V | |
| CDRH1 (IMGT) GFX₁FX₂X₃AW, where X₁ = T or I, | 613 |
| X₂ = N, I, or S, and X₃ = N, or Y | |
| CDRH2 (IMGT) IKX₁X₂X₃X₄GGTX₅, where X₁ = S, R, | 614 |
| or T, X₂ = I, E, K, L, N, or Q, X₃ = S or T, X₄ = Y or D, and | |
| X₅ = T or R | |
| CDRH3 (IMGT) X₁TGX₂VWX₃YGGSLDX₄, where X₁ = | 615 |
| S, T, I, or A, X₂ = S or G, X₃ = K, N, or R, and X₄ = F or Y | |
| CDRL1 (IMGT) QX₁IX₂RX₃, where X₁ = G, S, F, N, or | |
| I, X₂ = S or T, and X₃ = F, Y, or S | |
| CDRL2 (IMGT) X₁AS, where X₁ = A or G | |
| CDRL3 (IMGT) QQX₁YX₂X₃X₄X₅T, where X₁ = T or | 618 |
| S, X₂ = S, N, R, or G, X₃ = T or S, X₄ = P or S, and X₅ = L or V | |
| Variants of 130 | |
| CDRH1 (Kabat) X₁AWMS, where X₁ = Y, N, or T | 619 |
| CDRH2 (Kabat) RIKX₁X₂X₃X₄GGTTDYAX₅PVKG, | 620 |
| where X₁ = S or R, X₂ = E or K, X₃ = T, S, or I, X₄ = D or Y, | |
| and X₅ = A or S | |
| CDRH3 (Kabat) GSSWX₁X₂YX₃FDX₄, where X₁ = N, | 621 |
| H, S or Y, X₂ = V or L, X₃ = F or Y, and X₄ = F or Y | |
| CDRL1 (Kabat) RASQSX₁SX₂X₃LN, where X₁ = I or | 622 |
| M, X₂ = N, S, R or T, and X₃ = Y or H | |
| CDRL2 (Kabat) AAX₁X₂LX₃X₄, where X₁ = Y or S, X₂ = | |
| R, S, or N, X₃ = Q or E, and X₄ = T or S | |
| CDRL3 (Kabat) QQSYX₁X₂PX₃T, where X₁ = S or R, | 624 |
| X₂ = S or T, and X₃ = L or F | |
| CDRH1 (Chothia) GFX₁FSX₂A, where X₁ = T or I, and | 625 |
| X₂ = Y, N, or T | |
| CDRH2 (Chothia) KX₁X₂X₃X₄GGT, where X₁ = S or | 626 |
| R, X₂ = E or K, X₃ = T, S, or I, X₄ = D or Y | |
| CDRH3 (Chothia) SSWX₁X₂YX₃FD, X₁ = N, H, S or | 627 |
| Y, X₂ = V or L, and X₃ = F or Y | |
| CDRL1 (Chothia) SQSX₁SX₂X₃, where X₁ = I or M, X₂ = | 628 |
| N, S, R or T, and X₃ = Y or H | |
| CDRL2 (Chothia) AAX₁, where X₁ = Y or S | |
| CDRL3 (Chothia) SYX₁X₂PX₃, where X₁ = S or R, X₂ = | |
| S or T, and X₃ = L or F | |
| CDRH1 (IMGT) GFX₁FSX₂AW, where X₁ = T or I, and | 631 |
| X₂ = Y, N, or T | |
| CDRH2 (IMGT) IKX₁X₂X₃X₄GGTT, where X₁ = S or | 632 |
| R, X₂ = E or K, X₃ = T, S, or I, and X₄ = D or Y | |
| CDRH3 (IMGT) TTGSSWX₁X₂YX₃FDX₄, where X₁ = | 633 |
| N, H, S or Y, X₂ = V or L, X₃ = F or Y, and X₄ = F or Y | |
| CDRL1 (IMGT) QSX₁SX₂X₃, where X₁ = I or M, X₂ = | |
| N, S, R or T, and X₃ = Y or H | |
| CDRL2 (IMGT) AAX₁, where X₁ = Y or S | |
| CDRL3 (IMGT) QQSYX₁X₂PX₃T, where X₁ = S or R, | 636 |
| X₂ = S or T, and X₃ = L or F | |

In certain embodiments, any of the antibodies or antigen binding fragment thereof described above further comprises a heavy chain comprising a human IgG sequence selected from a sequence set forth in any one of SEQ ID NOs: 637-737 and 913-1012 or a sequence having at least about 80%, 90%, 91%, 92%, 93%, 94%, 95%, 96%, 97%, 98% or 99% identity thereto. In certain embodiments, any of the antibodies or antigen binding fragment thereof described above further comprises a heavy chain sequence set forth in SEQ ID NO: 651 or SEQ ID NO: 927 or a sequence having at least about 80%, 90%, 91%, 92%, 93%, 94%, 95%, 96%, 97%, 98% or 99% identity thereto. In certain embodiments, any of the antibodies or antigen binding fragment thereof described above further comprises a heavy chain sequence set forth in SEQ ID NO: 651 or a sequence having at least about 80%, 90%, 91%, 92%, 93%, 94%, 95%, 96%, 97%, 98% or 99% identity thereto. In certain embodiments, any of the antibodies or antigen binding fragment thereof described above further comprises a heavy chain sequence set forth in SEQ ID NO: 927 or a sequence having at least about 80%, 90%, 91%, 92%, 93%, 94%, 95%, 96%, 97%, 98% or 99% identity thereto. In certain embodiments, any of the antibodies or antigen binding fragment thereof described above further comprises a human light chain sequence set forth in SEQ ID NO: 738 or a sequence having at least about 80%, 90%, 91%, 92%, 93%, 94%, 95%, 96%, 97%, 98% or 99% identity thereto. In certain embodiments, any of the antibodies or antigen binding fragment thereof described above further comprises a heavy chain sequence set forth in SEQ ID NO: 651 or a sequence having at least about 80%, 90%, 91%, 92%, 93%, 94%, 95%, 96%, 97%, 98% or 99% identity thereto and a human light chain sequence set forth in SEQ ID NO: 738 or a sequence having at least about 80%, 90%, 91%, 92%, 93%, 94%, 95%, 96%, 97%, 98% or 99% identity thereto. In certain embodiments, any of the antibodies or antigen binding fragment thereof described above further comprises a heavy chain sequence set forth in SEQ ID NO: 927 or a sequence having at least about 80%, 90%, 91%, 92%, 93%, 94%, 95%, 96%, 97%, 98% or 99% identity thereto and a human light chain sequence set forth in SEQ ID NO: 738 or a sequence having at least about 80%, 90%, 91%, 92%, 93%, 94%, 95%, 96%, 97%, 98% or 99% identity thereto.

In some embodiments, the antibody, or antigen binding fragment thereof, comprises a CDR-H1 comprising the sequence set forth in any one of SEQ ID NOs: 1, 22, and 44; a CDR-H2 comprising the sequence set forth in any one of SEQ ID NOs: 70, 111, and 142; a CDR-H3 comprising the sequence set forth in any one of SEQ ID NOs: 175, 203, and 223; a CDR-L1 comprising the sequence set forth in any one of SEQ ID NOs: 258, 286, and 311; a CDR-L2 comprising the sequence set forth in any one of SEQ ID NOs: 335 and 359; and a CDR-L3 comprising the sequence set forth in any one of SEQ ID NOs: 368 and 394. In certain embodiments, the antibody further comprises a heavy chain constant region (e.g., an IgG1 constant region) comprising LALA/YTE substitutions. In certain embodiments, the antibody comprises a heavy chain comprising a heavy chain constant region selected from SEQ ID NO: 927 and SEQ ID NO: 651. In certain embodiments, the antibody comprises a heavy chain comprising a heavy chain constant region set forth in SEQ ID NO: 927. In certain embodiments, the antibody comprises a human kappa light chain constant region. In some embodiments, the antibody comprises a constant light chain sequence comprising a sequence set forth in SEQ ID NO: 738.

In some embodiments, the antibody, or antigen binding fragment thereof, comprises a CDR-H1 comprising an amino acid sequence set forth in any one of SEQ ID NOs: 8, 26, and 51; a CDR-H2 comprising an amino acid sequence set forth in any one of SEQ ID NOs: 752, 756, and 761; a CDR-H3 comprising an amino acid sequence set forth in any one of SEQ ID NOs: 767, 773, and 779, a CDR-L1 comprising an amino acid sequence set forth in any one of SEQ ID NOs: 785, 790, and 795; a CDR-L2 comprising an amino acid sequence set forth in any one of SEQ ID NOs: 800 and 805; and a CDR-L3 comprising an amino acid sequence set forth in any one of SEQ ID NOs: 810 and 816. In certain embodiments, the antibody further comprises a heavy chain constant region (e.g., an IgG1 constant region) comprising LALA/YTE substitutions. In certain embodiments, the antibody comprises a heavy chain comprising a heavy chain constant region selected from SEQ ID NO: 927 and SEQ ID NO: 651. In certain embodiments, the antibody comprises a heavy chain comprising a heavy chain constant region set forth in SEQ ID NO: 927. In certain embodiments, the antibody comprises a human kappa light chain constant region. In some embodiments, the antibody comprises a constant light chain sequence comprising a sequence set forth in SEQ ID NO: 738.

In some embodiments, the antibody, or antigen binding fragment thereof, comprises a CDR-H1 comprising an amino acid sequence set forth in any one of SEQ ID NOs: 741, 744, and 747; a CDR-H2 comprising an amino acid sequence set forth in any one of SEQ ID NOs: 753, 757, and 762; a CDR-H3 comprising an amino acid sequence set forth in any one of SEQ ID NOs: 768, 774, and 780, a CDR-L1 comprising an amino acid sequence set forth in any one of SEQ ID NOs: 786, 791, and 796; a CDR-L2 comprising an amino acid sequence set forth in any one of SEQ ID NOs: 801 and 360; and a CDR-L3 comprising an amino acid sequence set forth in any one of SEQ ID NOs: 811 and 817. In certain embodiments, the antibody further comprises a heavy chain constant region (e.g., an IgG1 constant region) comprising LALA/YTE substitutions. In certain embodiments, the antibody comprises a heavy chain comprising a heavy chain constant region selected from SEQ ID NO: 927 and SEQ ID NO: 651. In certain embodiments, the antibody comprises a heavy chain comprising a heavy chain constant region set forth in SEQ ID NO: 927. In certain embodiments, the antibody comprises a human kappa light chain constant region. In some embodiments, the antibody comprises a constant light chain sequence comprising a sequence set forth in SEQ ID NO: 738.

In some embodiments, the antibody, or antigen binding fragment thereof, comprises a CDR-H1 comprising an amino acid sequence set forth in any one of SEQ ID NOs: 10, 27, and 52; a CDR-H2 comprising an amino acid sequence set forth in any one of SEQ ID NOs: 70, 111, and 142; a CDR-H3 comprising an amino acid sequence set forth in any one of SEQ ID NOs: 175, 203, and 234, a CDR-L1 comprising an amino acid sequence set forth in any one of SEQ ID NOs: 260, 288, and 313; a CDR-L2 comprising an amino acid sequence set forth in any one of SEQ ID NOs: 347 and 365; and a CDR-L3 comprising an amino acid sequence set forth in any one of SEQ ID NOs: 378 and 404. In certain embodiments, the antibody further comprises a heavy chain constant region (e.g., an IgG1 constant region) comprising LALA/YTE substitutions. In certain embodiments, the antibody comprises a heavy chain comprising a heavy chain constant region selected from SEQ ID NO: 927 and SEQ ID NO: 651. In certain embodiments, the antibody comprises a heavy chain comprising a heavy chain constant region set forth in SEQ ID NO: 927. In certain embodiments, the antibody comprises a human kappa light chain constant region. In some embodiments, the antibody comprises a constant light chain sequence comprising a sequence set forth in SEQ ID NO: 738.

Although the antibody may be produced using a polynucleotide sequence that encodes a constant heavy chain region containing a C-terminal lysine (e.g., a sequence encoding any one of SEQ ID NOs: 913-1012), the C-terminal lysine may be cleaved off during manufacture or after administration (resulting in, e.g., an antibody containing a constant heavy chain sequence of any one of SEQ ID NOs: 637-737). Accordingly, any of the antibodies or antigen binding fragments thereof described above may comprise a human IgG sequence containing a C-terminal lysine (e.g., any one of SEQ ID NOs: 913-1012)), a human IgG sequence lacking a C-terminal lysine (e.g., any one of SEQ ID NOs: 637-737), or a mixture thereof (e.g., a mixture of the same heavy chain constant region sequence with and without a C-terminal lysine, such as a mixture of SEQ ID NO: 927 and SEQ ID NO: 651).

In some aspects, the amino acid substitutions are conservative amino acid substitutions. In some embodiments, the antibodies described in this disclosure are referred to herein as “variants” or “clones”. In some embodiments, such variants or clones are derived from a sequence provided herein, for example, by affinity maturation, site directed mutagenesis, random mutagenesis, or any other method known in the art or described herein. In some embodiments, such variants or clones are not derived from a sequence provided herein and may, for example, be isolated de novo according to the methods provided herein for obtaining antibodies.

### Fc Region

The structures of the Fc regions of various immunoglobulins, and the glycosylation sites contained therein, are known in the art. See Schroeder et al. (2010) Allergy Clin. Immunol. 125:S41-52, incorporated by reference in its entirety. The Fc region may be a naturally occurring Fc region, or an Fc region modified as described in the art or elsewhere in this disclosure.

Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, MD, 1991. An “Fc polypeptide” of a dimeric Fc as used herein refers to one of the two polypeptides forming the dimeric Fc domain, i.e. a polypeptide comprising C-terminal constant regions of an immunoglobulin heavy chain, capable of stable self-association. For example, an Fc polypeptide of a dimeric IgG Fc comprises an IgG CH2 and an IgG CH3 constant domain sequence. An Fc can be of the class IgA, IgD, IgE, IgG, and IgM, and several of these may be further divided into subclasses (isotypes), e.g., IgG₁, IgG₂, IgG₃, IgG₄, IgA₁, and IgA₂.

In certain embodiments, provided human IgG1 Fc regions include an SRDEL (SEQ ID NO: 1016) allotype or an SREEM (SEQ ID NO: 1017) allotype.

The terms “Fc receptor” and “FcR” are used to describe a receptor that binds to the Fc region of an antibody. For example, an FcR can be a native sequence human FcR. Generally, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 1999)). Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976); and Kim et al., J. Immunol. 24:249 (1994)).

Modifications in the CH2 domain can affect the binding of FcRs to the Fc. A number of amino acid modifications in the Fc region are known in the art for selectively altering the affinity of the Fc for different Fcgamma receptors. In some aspects, the Fc comprises one or more modifications to promote selective binding of Fc-gamma receptors.

Exemplary mutations that may alter the binding of FcRs to the Fc are listed below (in EU numbering format):
S298A/E333A/K334A, S298A/E333A/K334A/K326A (Lu Y, Vernes J M, Chiang N, et al., J Immunol Methods. 2011 Feb. 28; 365(1-2):132-41);
F243L/R292P/Y300L/V305I/P396L, F243L/R292P/Y300L/L235V/P396L (Stavenhagen J B, Gorlatov S, Tuaillon N, et al., Cancer Res. 2007 Sep. 15; 67(18):8882-90; Nordstrom J L, Gorlatov S, Zhang W, et al., Breast Cancer Res. 2011 Nov. 30; 13(6):R123);
F243L (Stewart R, Thom G, Levens M, et al., Protein Eng Des Sel. 2011 September; 24(9):671-8.), S298A/E333A/K334A (Shields R L, Namenuk A K, Hong K, et al., J Biol Chem. 2001 Mar. 2; 276(9):6591-604);
S239D/I332E/A330L, S239D/I332E (Lazar et al., (2006)Proc Natl Acad Sci USA. 103(11):4005-10);
S239D/S267E, S267E/L328F (Chu et al., (2008) Mol Immunol. 45(15):3926-33);
S239D/D265S/S298A/I332E, S239E/S298A/K326A/A327H, G237F/S298A/A330L/I332E, S239D/I332E/S298A, S239D/K326E/A330L/I332E/S298A, G236A/S239D/D270L/I332E, S239E/S267E/H268D, L234F/S267E/N325L, G237F/V266L/S267D and other mutations listed in WO2011/120134 and WO2011/120135, herein incorporated by reference. Therapeutic Antibody Engineering (by William R. Strohl and Lila M. Strohl, Woodhead Publishing series in Biomedicine No 11, ISBN 1 907568 37 9, October 2012) lists mutations on page 283.

In some embodiments, the heavy chain comprises a constant heavy chain sequence selected from an amino acid sequence set forth in any one of SEQ ID NOs: 637-737 and 913-1012, or an amino acid sequence having at least 90%, at least 91%, at least 92%, at least 93%, at least 94%, at least 95%, at least 96%, at least 97%, at least 98%, or at least 99% sequence identity thereto. In some embodiments, the heavy chain comprises a constant heavy chain sequence set forth in SEQ ID NO: 651 or SEQ ID NO: 927, or an amino acid sequence having at least 90%, at least 91%, at least 92%, at least 93%, at least 94%, at least 95%, at least 96%, at least 97%, at least 98%, or at least 99% sequence identity thereto. In some embodiments, the heavy chain comprises a constant heavy chain sequence set forth in SEQ ID NO: 651, or an amino acid sequence having at least 90%, at least 91%, at least 92%, at least 93%, at least 94%, at least 95%, at least 96%, at least 97%, at least 98%, or at least 99% sequence identity thereto. In some embodiments, the heavy chain comprises a constant heavy chain sequence set forth in SEQ ID NO: 927, or an amino acid sequence having at least 90%, at least 91%, at least 92%, at least 93%, at least 94%, at least 95%, at least 96%, at least 97%, at least 98%, or at least 99% sequence identity thereto. In some embodiments, the light chain comprises a constant light chain sequence set forth in SEQ ID NO: 738, or an amino acid sequence having at least 90%, at least 91%, at least 92%, at least 93%, at least 94%, at least 95%, at least 96%, at least 97%, at least 98%, or at least 99% sequence identity thereto. In some embodiments, the heavy chain comprises a constant heavy chain sequence set forth in SEQ ID NO: 651, or an amino acid sequence having at least 90%, at least 91%, at least 92%, at least 93%, at least 94%, at least 95%, at least 96%, at least 97%, at least 98%, or at least 99% sequence identity thereto and the light chain comprises a constant light chain sequence set forth in SEQ ID NO: 738, or an amino acid sequence having at least 90%, at least 91%, at least 92%, at least 93%, at least 94%, at least 95%, at least 96%, at least 97%, at least 98%, or at least 99% sequence identity thereto. In some embodiments, the heavy chain comprises a constant heavy chain sequence set forth in SEQ ID NO: 927, or an amino acid sequence having at least 90%, at least 91%, at least 92%, at least 93%, at least 94%, at least 95%, at least 96%, at least 97%, at least 98%, or at least 99% sequence identity thereto and the light chain comprises a constant light chain sequence set forth in SEQ ID NO: 738, or an amino acid sequence having at least 90%, at least 91%, at least 92%, at least 93%, at least 94%, at least 95%, at least 96%, at least 97%, at least 98%, or at least 99% sequence identity thereto.

In some embodiments, an antibody described herein includes modifications intended to improve its ability to mediate effector function. Such modifications that can have this effect are known in the art and include afucosylation, or engineering of the affinity of the Fc towards an activating receptor, mainly FCGR3a for ADCC, and towards C1q for CDC. The following TABLE 5 summarizes various designs reported in the literature for effector function engineering.

Methods of producing antibodies with little or no fucose on the Fc glycosylation site (Asn 297 EU numbering) without altering the amino acid sequence are well known in the art. The GlymaX® technology (ProBioGen AG) is based on the introduction of a gene for an enzyme which deflects the cellular pathway of fucose biosynthesis into cells used for antibody production. This prevents the addition of the sugar “fucose” to the N-linked antibody carbohydrate part by antibody-producing cells. (von Horsten et al. (2010) Glycobiology. 20 (12):1607-18). Examples of cell lines capable of producing defucosylated antibody include CHO-DG44 with stable overexpression of the bacterial oxidoreductase GDP-6-deoxy-D-lyxo-4-hexylose reductase (RMD) (see von Horsten et al. (2010) supra) or Lec13 CHO cells, which are deficient in protein fucosylation (see Ripka et al. (1986) Arch. Biochem. Biophys. 249:533-545; U.S. Pat. Pub. No. 2003/0157108; WO 2004/056312; each of which is incorporated by reference in its entirety), and knockout cell lines, such as alpha-1,6-fucosyltransferase gene or FUT8 knockout CHO cells (see Yamane-Ohnuki et al. (2004) Biotech. Bioeng. 87: 614-622; Kanda et al. (2006) Biotechnol. Bioeng. 94:680-688; and WO 2003/085107; each of which is incorporated by reference in its entirety). Another approach to obtaining antibodies with lowered levels of fucosylation can be found in U.S. Pat. No. 8,409,572, which teaches selecting cell lines for antibody production for their ability to yield lower levels of fucosylation on antibodies.

Antibodies can be fully afucosylated (meaning they contain no detectable fucose) or they can be partially afucosylated, meaning that the isolated antibody contains less than 95%, less than 85%, less than 75%, less than 65%, less than 55%, less than 45%, less than 35%, less than 25%, less than 15% or less than 5% of the amount of fucose normally detected for a similar antibody produced by a mammalian expression system.

In some aspects, an antibody, or an antigen binding fragment thereof, provided herein comprises an IgG1 domain with reduced fucose content at position Asn 297 compared to a naturally occurring IgG1 domain. Such Fc domains can have improved ADCC. See Shields et al. (2002) J. Biol. Chem. 277:26733-26740, incorporated by reference in its entirety. In some aspects, such antibodies do not comprise any fucose at position Asn 297. The amount of fucose may be determined using any suitable method, for example as described in WO 2008/077546, incorporated by reference in its entirety.

In certain embodiments, an antibody, or an antigen binding fragment thereof, provided herein comprises an Fc region with one or more amino acid substitutions which improve ADCC, such as a substitution at one or more of positions 298, 333, and 334 of the Fc region. In some embodiments, an antibody, or an antigen binding fragment thereof, provided herein comprises an Fc region with one or more amino acid substitutions at positions 239, 332, and 330, as described in Lazar et al. (2006) Proc. Natl. Acad. Sci. USA 103:4005-4010, incorporated by reference in its entirety.

Other illustrative glycosylation variants which may be incorporated into the antibodies provided herein are described, for example, in U.S. Pat. Pub. Nos. 2003/0157108, 2004/0093621, 2003/0157108, 2003/0115614, 2002/0164328, 2004/0093621, 2004/0132140, 2004/0110704, 2004/0110282, 2004/0109865; International Pat. Pub. Nos. 2000/61739, 2001/29246, 2003/085119, 2003/084570, 2005/035586, 2005/035778; 2005/053742, 2002/031140; Okazaki et al., J. Mol. Biol., 2004, 336:1239-1249; and Yamane-Ohnuki et al. (2004) supra; each of which is incorporated by reference in its entirety.

In some embodiments, an antibody, or an antigen binding fragment thereof, provided herein comprises an Fc region with at least one galactose residue in the oligosaccharide attached to the Fc region. Such antibody variants may have improved CDC function. Examples of such antibody variants are described, for example, in WO 1997/30087; WO 1998/58964; and WO 1999/22764; each of which is incorporated by reference in its entirety.

Thus, in one embodiment, an antibody described herein can include a dimeric Fc that comprises one or more amino acid modifications as noted in TABLE 5 that may confer improved effector function. In another embodiment, the antibody can be afucosylated to improve effector function.

TABLE 5
CH2 domains and effector function engineering
TABLE 4

| Reference | Mutations | Effect |
|---|---|---|
| Lu (2011), supra; | Afucosylated | Increased ADCC |
| Ferrara (2011), supra; | | |
| Mizushima et al. (2011) | | |
| Genes Cells. 16(11): | | |
| 1071-1080. | | |
| Lu (2011), supra | S298A/E333A/K334A | Increased ADCC |
| Lu (2011), supra | S298A/E333A/K334A/ | Increased ADCC |
| | K326A | |
| Stavenhagen (2007), | F243L/R292P/Y300L/ | Increased ADCC |
| supra | V305I/P396L | |
| Nordstrom (2011), supra | F243L/R292P/Y300L/ | Increased ADCC |
| | L235V/P396L | |
| Stewart (2011), supra | F243L | Increased ADCC |
| Shields (2001), supra | S298A/E333A/K334A | Increased ADCC |
| Lazar (2006), supra | S239D/I332E/A330L | Increased ADCC |
| Lazar (2006), supra | S239D/I332E | Increased ADCC |
| Bowles et al., (2006) | AME-D, not specified | Increased ADCC |
| Blood 108(8): 2648-54 | mutations | |
| Heider et al., (2011) | 37.1, mutations not | Increased ADCC |
| Blood 118(15): 4159-68 | disclosed | |
| Moore et al., (2010) | S267E/H268F/S324T | Increased CDC |
| MAbs 2(2): 181-9 | | |

Fc modifications intended to reduce FcgR and/or complement binding and/or effector function are known in the art. Recent publications describe strategies that have been used to engineer antibodies with reduced or silenced effector activity (see Strohl, W R (2009) Curr Opin Biotech 20:685-691, and Strohl, W R and Strohl L M, “Antibody Fc engineering for optimal antibody performance” In Therapeutic Antibody Engineering, Cambridge: Woodhead Publishing (2012), pp 225-249). These strategies include reduction of effector function through modification of glycosylation, use of IgG2/IgG4 scaffolds, or the introduction of mutations in the hinge or CH2 regions of the Fc. For example, U.S. Patent Publication No. 2011/0212087 (Strohl), International Patent Publication No. WO 2006/105338 (Xencor), U.S. Patent Publication No. 2012/0225058 (Xencor), U.S. Patent Publication No. 2012/0251531 (Genentech), and Strop et al ((2012) J. Mol. Biol. 420: 204-219), each of which is incorporated by reference in its entirety, describe specific modifications designed to reduce FcgR or complement binding to the Fc.

Specific, non-limiting examples of amino acid modifications intended to reduce FcgR or complement binding to the Fc include those identified in the following TABLE 6:

TABLE 6
Modifications designed to reduce FcgR or complement binding to the Fc
Mutations

N297A
L234A/L235A
IGG2 V234A/G237A
IGG4 L235A/G237A/E318A
IGG4 S228P/L236E
IGG2/IGG4
IGG2 H268Q/V309L/A330S/A331S
C220S/C226S/C229S/P238S
C226S/C229S/E3233P/L235V/L235A
E. coli production, non glyco
L234F/L235E/P331S
Hinge mutant, e.g., C226S/P230S
D265A
L235A/G237A
L234A/L235A/G237A
L234A/L235A/P329G

In some embodiments, an antibody provided herein comprises one or more alterations that is designed to improve or diminish C1q binding and/or CDC. See U.S. Pat. No. 6,194,551; WO 99/51642; and Idusogie et al. (2000) J. Immunol. 164:4178-4184; each of which is incorporated by reference in its entirety.

In certain embodiments, an antibody provided herein comprises a heavy chain comprising a constant heavy chain sequence selected from the sequences set forth in SEQ ID NOs: 637-737 and 913-1012. In certain embodiments, the constant heavy chain sequence, (e.g., a constant heavy chain sequence selected from SEQ ID NOs: 637-737) further comprises a C-terminal lysine (e.g., SEQ ID NOs: 913-1012). Although the antibody may be produced using a polynucleotide sequence that encodes a constant heavy chain region containing a C-terminal lysine, the C-terminal lysine may be cleaved off during manufacture or after administration. Accordingly, sequences of heavy chain constant regions with and without the C-terminal lysine are provided herein. Consequently, a composition resulting from the manufacture of an antibody using a polynucleotide sequence that encodes a constant heavy chain region containing a C-terminal lysine (e.g., an antibody with a coding sequence that encodes any one of SEQ ID NOs: 913-1012) may comprise antibodies having a constant heavy chain sequence containing a C-terminal lysine (e.g., selected from SEQ ID NOs: 913-1012), antibodies having a constant heavy chain sequence lacking a C-terminal lysine (e.g., the corresponding sequence of any one of SEQ ID NOs: 637-737), or a mixture thereof. For example, a composition comprising an antibody comprising the constant heavy chain sequence of SEQ ID NO: 927 that is administered to a subject may comprise antibodies having the constant heavy chain sequence set forth in SEQ ID NO: 927 or SEQ ID NO: 651 or a mixture thereof (e.g., a mixture of antibodies having either a constant heavy chain sequence of SEQ ID NO: 927 or a constant heavy chain sequence of SEQ ID NO: 651 and/or antibodies containing both constant heavy chain sequences (e.g., in a single antibody containing two constant heavy chain sequences)).

In certain embodiments, an antibody provided herein comprises a VH sequence and a VL sequence provided in TABLE 3 with a heavy chain constant region selected from any one of SEQ ID NOs: 637-737 and 913-1012. In certain embodiments, an antibody provided herein comprises a VH sequence and a VL sequence provided in TABLE 3 with a heavy chain constant region selected from any one of SEQ ID NOs: 637-737 and 913-1012 and a light chain constant region set forth in SEQ ID NO: 738.

In certain embodiments, the antibody, or antigen binding fragment thereof, comprises a VH sequence set forth in SEQ ID NO: 419 and a VL sequence set forth in SEQ ID NO: 489; and the constant heavy chain comprises a human IgG sequence selected from a sequence set forth in any one of SEQ ID NOs: 637-737 and 913-1012 (e.g., SEQ ID NO: 927 or SEQ ID NO: 651). In certain embodiments, the antibody, or antigen binding fragment thereof, comprises a light chain constant region set forth in SEQ ID NO: 738.

In certain embodiments, the anti-IL-OX40L antibody comprises a heavy chain comprising an amino acid sequence set forth in SEQ ID NO: 1013 and/or SEQ ID NO: 1014. In certain embodiments, the anti-OX40L antibody comprises a light chain comprising an amino acid sequence set forth in SEQ ID NO: 1015. In certain embodiments, the anti-OX40L antibody comprises a heavy chain comprising an amino acid sequence set forth in SEQ ID NO: 1013 and a light chain comprising an amino acid sequence set forth in SEQ ID NO: 1015. The heavy chain sequence of SEQ ID NO: 1013 contains a C-terminal lysine, which may be cleaved off during manufacture or after administration, resulting in an antibody containing the heavy chain sequence of SEQ ID NO: 1014. In certain embodiments, the anti-OX40L antibody comprises a heavy chain comprising an amino acid sequence set forth in SEQ ID NO: 1014 and a light chain comprising an amino acid sequence set forth in SEQ ID NO: 1015.

In certain embodiments, the Fc region comprises one or more amino acid substitutions, wherein the one or more amino acid substitutions result in an increase in one or more of antibody half-life, ADCC activity, ADCP activity, or CDC activity compared with the Fc region without the one or more amino acid substitutions. In certain embodiments, the one or more amino acid substitutions results in increased antibody half-life at pH 6.0 compared to an antibody comprising a wild-type Fc region. In certain embodiments, the antibody has an increased half-life that is about 10,000-fold, 1,000-fold, 500-fold, 100-fold, 50-fold, 20-fold, 10-fold, 9-fold, 8-fold, 7-fold, 6-fold, 5-fold, 4.5-fold, 4-fold, 3.5-fold, 3-fold, 2.5-fold, 2-fold, 1.95-fold, 1.9-fold, 1.85-fold, 1.8-fold, 1.75-fold, 1.7-fold, 1.65-fold, 1.6-fold, 1.55-fold, 1.50-fold, 1.45-fold, 1.4-fold, 1.35-fold, 1.3-fold, 1.25-fold, 1.2-fold, 1.15-fold, 1.1-fold, or 1.05-fold longer compared to an antibody comprising a wild-type Fc region. In certain embodiments, the antibody has an increased half-life that is about 10,000-fold, 1,000-fold, 500-fold, 100-fold, 50-fold, 20-fold, 10-fold, 9-fold, 8-fold, 7-fold, 6-fold, 5-fold, 4.5-fold, 4-fold, 3.5-fold, 3-fold, 2.5-fold, 2-fold, 1.95-fold, 1.9-fold, 1.85-fold, 1.8-fold, 1.75-fold, 1.7-fold, 1.65-fold, 1.6-fold, 1.55-fold, 1.50-fold, 1.45-fold, 1.4-fold, 1.35-fold, 1.3-fold, 1.25-fold, 1.2-fold, 1.15-fold, 1.1-fold, or 1.05-fold longer compared to amlitelimab.

In certain embodiments, an antibody described herein comprises a heavy chain constant domain having a means for increasing the half-life of the antibody.

In certain embodiments, the Fc region comprises one or more amino acid substitutions, wherein the one or more amino acid substitutions result in a decrease in one or more of ADCC activity, ADCP activity, or CDC activity compared with the Fc region without the one or more amino acid substitutions.

In certain embodiments, the one or more amino acid substitutions is selected from the group consisting of S228P (SP), M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W. In certain embodiments, the one or more amino acid substitutions comprises a specific combination of amino acid substitutions selected from the group consisting of M428L/N434S (LS), M252Y/S254T/T256E (YTE), T250Q/M428L, T307A/E380A/N434A, T256D/T307Q (DQ), T256D/T307W (DW), M252Y/T256D (YD), T307Q/Q311V/A378V (QVV), T256D/H285D/T307R/Q311V/A378V (DDRVV), L309D/Q311H/N434S (DHS), S228P/L235E (SPLE), L234A/L235A (LALA), M428L/N434A (LA), L235A/G237A (LAGA), L234A/L235A/G237A (LALAGA), L234A/L235A/P329G (LALAPG), D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/N434A, D265A/N434A, LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV.

Although the EU numbering system is typically used to identify the positions of the various Fc mutations described herein, direct numbering can also be used. For example, in certain embodiments, an antibody described herein comprises an Fc region with YTE mutations at positions 253, 255 and 257, respectively. In certain embodiments, an antibody described herein comprises an Fc region with LALA mutations at positions 235 and 236, respectively. In certain embodiments, an antibody described herein comprises an Fc region (e.g., a human IgG1 Fc region) with YTE mutations at positions 253, 255 and 257 and with LALA mutations at positions 235 and 236, respectively. In certain embodiments, an OX40L antibody described herein comprises the VH and VL of Construct 114 and an Fc region comprising YTE mutations at positions 253, 255 and 257 (M253Y/S255T/T257E), respectively and with LALA mutations at positions 235 and 236 (L235A/L236A), respectively (e.g., as shown in the heavy chain sequences of SEQ ID NOs: 1013 and 1014).

In certain embodiments the human Fc region comprises a human IgG1 Fc region with LALA mutations. In certain embodiments, the human Fc region comprises a human IgG1 Fc region with YTE mutations. In certain embodiments, the human Fc region comprises a human IgG1 Fc region with LALA and YTE mutations. In certain embodiments, when direct numbering is used, “YTE” and “LALA” mutations can be located at different amino acid position numbers. For example, a human Fc region can comprise a human IgG1 Fc region with LALA mutations at L235A/L236A and/or YTE mutations at M253Y/S255T/T257E.

In certain embodiments, the Fc region binds an Fc Receptor selected from the group consisting of: FcγRI, FcγRIIa, FcγRIIb, FcγRIIc, FcγRIIIa, and FcγRIIIb. In certain embodiments, the Fc region binds an Fc Receptor with higher affinity at pH 6.0 compared to an antibody comprising a wild-type Fc region.

In some embodiments, an antibody disclosed herein comprises a VH and/or VL sequence from TABLE 7. In some embodiments, an antibody containing a VH and/or VL sequence of TABLE 7 also comprises one or more amino acid substitutions, e.g., substitutions in the Fc region, disclosed herein. In some embodiments, an antibody containing a VH and/or VL sequence of TABLE 7 comprises a constant heavy chain sequence selected from a sequences set forth in any one of SEQ ID NOs: 637-737 and 913-1012. In some embodiments, an antibody comprising a VH and/or VL sequence of TABLE 7 comprises one or more amino acid substitutions selected from the group consisting of S228P (SP), M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W. In certain embodiments, an antibody containing a VH and/or VL sequence of TABLE 7 comprises a specific combination of amino acid substitutions selected from the group consisting of M428L/N434S (LS), M252Y/S254T/T256E (YTE), T250Q/M428L, T307A/E380A/N434A, T256D/T307Q (DQ), T256D/T307W (DW), M252Y/T256D (YD), T307Q/Q311V/A378V (QVV), T256D/H285D/T307R/Q311V/A378V (DDRVV), L309D/Q311H/N434S (DHS), S228P/L235E (SPLE), L234A/L235A (LALA), M428L/N434A (LA), L235A/G237A (LAGA), L234A/L235A/G237A (LALAGA), L234A/L235A/P329G (LALAPG), D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/N434A, D265A/N434A, LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV.

TABLE 7
VH and VL sequences of anti-OX40L antibodies

| Antibody | VH | VL |
|---|---|---|
| Amlitelimab | EVQLVESGGGLVQPGGSLRLSC | DIQMTQSPSSLSASVGDRVTITC |
| | AASGFTFSNYAMNWVRQAPGK | RASQSISSYLNWYQQKPGKAP |
| | GLEWVSTISGSGGATRYADSVK | NLLIYAASSLQSGVPSRFSGSGS |
| | GRFTISRDNSRNTVYLQMNSLR | ETDFTLTISSLQPEDFATYYCQQ |
| | VEDTAVFYCTKDRLIMATVRGP | SHSVSFTFGPGTKVDIK |
| | YYYGMDVWGQGTTVTVSS | (SEQ ID NO: 833) |
| | (SEQ ID NO: 832) | |
| LC.001 | EVQLLESGGGLVQPGGSLRLSC | DIQMTQSPSSLSASVGDRVTITC |
| (Oxelumab) | AASGFTFNSYAMSWVRQAPGK | RASQGISSWLAWYQQKPEKAP |
| | GLEWVSIISGSGGFTYYADSVK | KSLIYAASSLQSGVPSRFSGSGS |
| | GRFTISRDNSRTTLYLQMNSLR | GTDFTLTISSLQPEDFATYYCQ |
| | AEDTAVYYCAKDRLVAPGTFD | QYNSYPYTFGQGTKLEIK |
| | YWGQGALVTVSS | (SEQ ID NO: 835) |
| | (SEQ ID NO: 834) | |
| LC.005 | QVQLVESGGGVVQPGRSLRLSC | EIVLTQSPGTLSLSPGERATLSC |
| | AASGFTFSNFGMHWVRQAPGK | RASQSVSSNYLAWYQQKPGQA |
| | GLEWVAAIWYDGHDKYYSYY | PRLLIYGASSRATGIPDRFSGSG |
| | VKGRFTISRDNSKNTLFLQMNS | SGTDFTLTISRLEPEDFAVYYC |
| | LRAEDTAVYYCARDSSSWYRY | QQYGSSFTFGPGTKVDIK |
| | FDYWGQGTLVTVSS | (SEQ ID NO: 837) |
| | (SEQ ID NO: 836) | |
| LC.019 | QKQLVEFGGGVVQPGRSLRLSC | EIVLTQSPATLSLSPGERATLSC |
| | AASGFTFSNYGMHWVRQAPGK | RASQGVSRYLAWYQQKPGQA |
| | GLWVAVIWNDGSNKYYVDSV | PRLLIYDASNRATGIPARVSGS |
| | KGRFIISRDNSKNTLYLQMNSL | GPGTDFTLTISSLEPEDFAVDYC |
| | RAEDTAVYYCARDRMGIYYYG | QQRSNWQYTFGQGTKLEIK |
| | MDVWGQGTTVTVSS | (SEQ ID NO: 839) |
| | (SEQ ID NO: 838) | |

### Binding

The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein, such as surface plasmon resonance (SPR) technology (e.g., BIACORE®) or biolayer interferometry (e.g., FORTEBIO®).

With regard to the binding of an antibody to a target molecule, the terms “bind,” “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction (e.g., with a non-target molecule). Specific binding can be measured, for example, by measuring binding to a target molecule (i.e., OX40L) and comparing it to binding to a non-target molecule. Specific binding can also be determined by competition with a control molecule that mimics the epitope recognized on the target molecule. In that case, specific binding is indicated if the binding of the antibody to the target molecule is competitively inhibited by the control molecule. In some embodiments, the affinity of an anti-OX40L antibody, or antigen binding fragment thereof, for a non-target molecule is less than about 50% of the affinity for OX40L. In some embodiments, the affinity of an anti-OX40L antibody, or antigen binding fragment thereof, for a non-target molecule is less than about 40% of the affinity for OX40L. In some embodiments, the affinity of an anti-OX40L antibody, or antigen binding fragment thereof, for a non-target molecule is less than about 30% of the affinity for OX40L. In some embodiments, the affinity of an anti-OX40L antibody, or antigen binding fragment thereof, for a non-target molecule is less than about 20% of the affinity for OX40L. In some embodiments, the affinity of an anti-OX40L antibody, or antigen binding fragment thereof, for a non-target molecule is less than about 10% of the affinity for OX40L. In some embodiments, the affinity of an anti-OX40L antibody, or antigen binding fragment thereof, for a non-target molecule is less than about 1% of the affinity for OX40L. In some embodiments, the affinity of an anti-OX40L antibody, or antigen binding fragment thereof, for a non-target molecule is less than about 0.1% of the affinity for OX40L.

When used herein in the context of two or more antibodies, the term “competes with” or “cross-competes with” indicates that the two or more antibodies compete for binding to an antigen (e.g., OX40L). In one exemplary assay, OX40L is coated on a surface and contacted with a first anti-OX40L antibody, after which a second anti-OX40L antibody is added. In another exemplary assay, a first anti-OX40L antibody is coated on a surface and contacted with OX40L, and then a second anti-OX40L antibody is added. If the presence of the first anti-OX40L antibody reduces binding of the second anti-OX40L antibody, in either assay, then the antibodies compete with each other. The term “competes with” also includes combinations of antibodies where one antibody reduces binding of another antibody, but where no competition is observed when the antibodies are added in the reverse order. However, in some embodiments, the first and second antibodies inhibit binding of each other, regardless of the order in which they are added. In some embodiments, one antibody reduces binding of another antibody to its antigen by at least 25%, at least 50%, at least 60%, at least 70%, at least 80%, at least 85%, at least 90%, at least 95%, or at least 99% as measured in a competitive binding assay. A skilled artisan can select the concentrations of the antibodies used in the competition assays based on the affinities of the antibodies for OX-40L and the valency of the antibodies. The assays described in this definition are illustrative, and a skilled artisan can utilize any suitable assay to determine if antibodies compete with each other. Suitable assays are described, for example, in Cox et al., “Immunoassay Methods,” in Assay Guidance Manual [Internet], Updated Dec. 24, 2014 (ncbi.nlm.nih.gov/books/NBK92434/; accessed Sep. 29, 2015); Silman et al. (2001) Cytometry 44:30-37; and Finco et al., (2011) J. Pharm. Biomed. Anal. 54:351-358; each of which is incorporated by reference in its entirety.

A test antibody competes with a reference antibody if an excess of a test antibody (e.g., at least 2×, 5×, 10×, 20×, or 100×) inhibits or blocks binding of the reference antibody by, e.g., at least 50%, 60%, 70%, 75%, 80%, 85%, 90%, 95%, or 99% as measured in a competitive binding assay. Antibodies identified by competition assay (competing antibody) include antibodies binding to the same epitope as the reference antibody and antibodies binding to an adjacent epitope sufficiently proximal to the epitope bound by the reference antibody for steric hindrance to occur. For example, a second, competing antibody can be identified that competes for binding to OX40L with a first antibody described herein. In certain instances, the second antibody can block or inhibit binding of the first antibody by, e.g., at least 50%, 60%, 70%, 75%, 80%, 85%, 90%, 95%, or 99% as measured in a competitive binding assay. In certain instances, the second antibody can displace the first antibody by greater than 50%, 60%, 70%, 75%, 80%, 85%, 90%, 95%, or 99%.

In certain embodiments, the antibody, or antigen binding fragment thereof, binds an OX40L sequence set forth in SEQ ID NO: 739.

In certain embodiments, the antibody, or antigen binding fragment thereof, binds to an OX40L sequence set forth in SEQ ID NO: 739 with a K_{D} of less than or equal to about 1, 2, 3, 4, 5, 6, 7, 8, or 9×10⁻⁹ M, as measured by surface plasmon resonance (SPR). In certain embodiments, the antibody, or antigen binding fragment thereof, binds to an OX40L sequence set forth in SEQ ID NO: 739 with a K_{D} of less than or equal to about 1×10⁻¹⁰ M, as measured by surface plasmon resonance (SPR). In certain embodiments, the antibody, or antigen binding fragment thereof, binds to human OX40L with a K_{D} of less than or equal to about 1×10⁻⁹ M, as measured by surface plasmon resonance (SPR).

In some embodiments, an antibody, or antigen binding fragment thereof, provided herein binds OX40L with a K_{D} of less than or equal to about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 1.95, 2, 2.5, 3, 3.5, 4, 4.5, 5, 6, 7, 8, 9, or 10×10⁻⁸ M, as measured by ELISA or any other suitable method known in the art. In some embodiments, an antibody, or antigen binding fragment thereof, provided herein binds OX40L with a K_{D} of less than or equal to about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 1.95, 2, 2.5, 3, 3.5, 4, 4.5, 5, 6, 7, 8, 9, or 10×10⁻⁹ M, as measured by ELISA or any other suitable method known in the art.

In some embodiments, the K_{D} of the antibody, or antigen binding fragment thereof, provided herein for the binding of OX40L is between about 0.001-0.01, 0.01-0.1, 0.01-0.05, 0.05-0.1, 0.1-0.5, 0.5-1, 0.25-0.75, 0.25-0.5, 0.5-0.75, 0.75-1, 0.75-2, 1.1-1.2, 1.2-1.3, 1.3-1.4, 1.4-1.5, 1.5-1.6, 1.6-1.7, 1.7-1.8, 1.8-1.9, 1.9-2, 1-2, 1-5, 2-7, 3-8, 3-5, 4-6, 5-7, 6-8, 7-9, 7-10, or 5-10×10⁻⁸ M, as measured by ELISA or any other suitable method known in the art. In some embodiments, an antibody, or antigen binding fragment thereof, provided herein binds OX40L with a K_{D} of less than or equal to about 1×10⁻⁸ M, or less than or equal to about 1×10⁻⁹ M as measured by ELISA or any other suitable method known in the art.

In some embodiments, the antibody, or antigen binding fragment thereof, provided herein binds OX40L with a K_{D} of less than or equal to about 10, 9, 8, 7, 6, 5, 4.5, 4, 3.5, 3, 2.5, 2, 1.98, 1.95, 1.9, 1.85, 1.8, 1.75, 1.7, 1.65, 1.6, 1.55, 1.50, 1.45, 1.4, 1.3, 1.2, 1.1, 1, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.01, 0.005, 0.001, 0.0005, or 0.0001×10⁻⁸ M, or less, as measured by ELISA or any other suitable method known in the art. In some embodiments, the antibody, or antigen binding fragment thereof, provided herein binds OX40L with a K_{D} between 5-3, 4-2, 3-1, 1.9-1.8, 1.8-1.7, 1.7-1.6, 1.6-1.5, 1.9-1.5, 1.5-1, 1-0.8, 1-0.5, 0.9-0.6, 0.7-0.4, 0.6-0.2, 0.5-0.3, 0.3-0.2, 0.2-0.1, 0.1-0.01, 0.01-0.001, or 0.001-0.0001×10⁻⁸M as measured by ELISA or any other suitable method known in the art.

In some embodiments, the antibody, or antigen binding fragment thereof, provided herein binds FcRn with an affinity at pH 7.4 compared to pH 6.0 at a ratio (pH 7.4/pH 6.0) of about 10,000, 1,000, 500, 100, 50, 20, 10, 9, 8, 7, 6, 5, 4.5, 4, 3.5, 3, 2.5, 2, 1.95, 1.9, 1.85, 1.8, 1.75, 1.7, 1.65, 1.6, 1.55, 1.50, 1.45, 1.4, 1.3, 1.2, 1.1, or 1.05, as measured by ELISA or any other suitable method known in the art. In some embodiments, the antibody, or antigen binding fragment thereof, provided herein binds FcRn with an affinity at pH 6.0 compared to pH 7.4 at a ratio (pH 6.0/pH 7.4) of about 1-0.8, 1-0.5, 0.9-0.6, 0.7-0.4, 0.6-0.2, 0.5-0.3, 0.3-0.2, 0.2-0.1, 0.1-0.01, 0.01-0.001, or 0.001-0.0001×10⁻⁸ M as measured by ELISA or any other suitable method known in the art.

### Function

“Effector functions” refer to those biological activities mediated by the Fc region of an antibody, which activities may vary depending on the antibody isotype. Examples of antibody effector functions include receptor ligand blocking, agonism, or antagonism, C1q binding to activate complement dependent cytotoxicity (CDC), Fc receptor binding to activate antibody-dependent cellular cytotoxicity (ADCC), and antibody dependent cellular phagocytosis (ADCP).

### Pharmaceutical Compositions

The present application provides compositions comprising the antibodies including pharmaceutical compositions comprising any one or more of the antibodies or antigen binding fragments thereof described herein with one or more pharmaceutically acceptable excipients. In some embodiments the composition is sterile. The pharmaceutical compositions generally comprise an effective amount of an antibody, or antigen binding fragment thereof. In some embodiments, the pharmaceutical compositions further comprise a hyaluronidase or a variant thereof.

These compositions can comprise, in addition to one or more of the antibodies or antigen binding fragments thereof disclosed herein, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution, or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

For the anti-OX40L antibody, or antigen binding fragment thereof, that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of the disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### Methods

### Methods of Preparation

Antibodies, or antigen binding fragments thereof, described herein can be produced using recombinant methods and compositions, e.g., as described in U.S. Pat. No. 4,816,567. In one embodiment, an isolated nucleic acid encoding an antibody, or antigen binding fragment thereof, described herein is provided. Such a nucleic acid may encode an amino acid sequence comprising the VL and/or an amino acid sequence comprising the VH of the antibody (e.g., the light and/or heavy chains of the antibody) or an amino acid sequence comprising the VHH of a single domain antibody. In a further embodiment, one or more vectors (e.g., expression vectors) comprising such a nucleic acid are provided. In one embodiment, the nucleic acid is provided in a multicistronic vector. In a further embodiment, a host cell comprising such a nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antigen-binding polypeptide construct, or (2) a first vector comprising a nucleic acid that encodes an amino acid sequence comprising the VL of the antigen-binding polypeptide construct and a second vector comprising a nucleic acid that encodes an amino acid sequence comprising the VH of the antigen-binding polypeptide construct. In one embodiment, the host cell is eukaryotic, e.g. a Chinese Hamster Ovary (CHO) cell, human embryonic kidney (HEK) cell, or lymphoid cell (e.g., Y0, NS0, Sp20 cell). In one embodiment, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of the antibody, a nucleic acid encoding an antibody, e.g., as described above, is isolated and inserted into one or more vectors for further cloning and/or expression in a host cell. Such a nucleic acid may be readily isolated and sequenced using conventional procedures (e.g., by using oligonucleotide probes that are capable of binding specifically to genes encoding the heavy and light chains of the antibody).

When an antibody or variant thereof is recombinantly produced by the host cells, the protein in certain embodiments is present at about 30%, about 25%, about 20%, about 15%, about 10%, about 5%, about 4%, about 3%, about 2%, or about 1% or less of the dry weight of the cells. When the antibody or variant thereof is recombinantly produced by the host cells, the protein, in certain embodiments, is present in the culture medium at about 5 g/L, about 4 g/L, about 3 g/L, about 2 g/L, about 1 g/L, about 750 mg/L, about 500 mg/L, about 250 mg/L, about 100 mg/L, about 50 mg/L, about 10 mg/L, or about 1 mg/L or less of the dry weight of the cells. In certain embodiments, a “substantially purified” antibody produced by the methods described herein has a purity level of at least about 30%, at least about 35%, at least about 40%, at least about 45%, at least about 50%, at least about 55%, at least about 60%, at least about 65%, at least about 70%, specifically, a purity level of at least about 75%, at least about 80%, at least about 85%, and more specifically, a purity level of at least about 90%, a purity level of at least about 95%, a purity level of at least about 99% or greater as determined by appropriate methods such as SDS/PAGE analysis, RP-HPLC, SEC, and capillary electrophoresis.

Suitable host cells for cloning or expression of antibody-encoding vectors include prokaryotic or eukaryotic cells described herein.

Recombinant host cells or host cells are cells that include an exogenous polynucleotide, regardless of the method used for insertion, for example, direct uptake, transduction, f-mating, or other methods known in the art to create recombinant host cells. The exogenous polynucleotide may be maintained as a nonintegrated vector, for example, a plasmid, or alternatively, may be integrated into the host genome. Host cells can include CHO, derivatives of CHO, NS0, Sp20, CV-1, VERO-76, HeLa, HepG2, Per.C6, or BHK.

For example, an antibody may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibodies are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al. (1980) Proc. Natl. Acad. Sci. USA 77:4216); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

In one embodiment, the antibodies described herein are produced in stable mammalian cells, by a method comprising: transfecting at least one stable mammalian cell with: nucleic acid encoding the antibody, in a predetermined ratio; and expressing the nucleic acid in the at least one mammalian cell. In some embodiments, the predetermined ratio of nucleic acid is determined in transient transfection experiments to determine the relative ratio of input nucleic acids that results in the highest percentage of the antibody in the expressed product.

In some embodiments, the method of producing an antibody in stable mammalian cells as described herein results in an expression product of the at least one stable mammalian cell comprising a larger percentage of the desired glycosylated antibody as compared to the monomeric heavy or light chain polypeptides, or other antibodies.

In some embodiments, the method of producing a glycosylated antibody in stable mammalian cells described herein comprises identifying and purifying the desired glycosylated antibody. In some embodiments, the said identification is by one or both of liquid chromatography and mass spectrometry.

If required, the antibodies can be purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc and antibodies, and these proteins can find use in the present invention for purification of antibodies. For example, the bacterial proteins A and G bind to the Fc region. Likewise, the bacterial protein L binds to the Fab region of some antibodies. Purification can often be enabled by a particular fusion partner. For example, antibodies may be purified using glutathione resin if a GST fusion is employed, Ni⁺² affinity chromatography if a His-tag is employed or immobilized anti-flag antibody if a flag-tag is used. For general guidance in suitable purification techniques, see, e.g. incorporated entirely by reference Protein Purification: Principles and Practice, 3rd Ed., Scopes, Springer-Verlag, NY, 1994, incorporated entirely by reference. The degree of purification necessary will vary depending on the use of the antibodies. In some instances, no purification is necessary.

In certain embodiments, the antibodies are purified using Anion Exchange Chromatography including, but not limited to, chromatography on Q-sepharose, DEAE sepharose, poros HQ, poros DEAF, Toyopearl Q, Toyopearl QAE, Toyopearl DEAE, Resource/Source Q and DEAE, Fractogel Q and DEAE columns.

In specific embodiments, the proteins described herein are purified using Cation Exchange Chromatography including, but not limited to, SP-sepharose, CM sepharose, poros HS, poros CM, Toyopearl SP, Toyopearl CM, Resource/Source S and CM, Fractogel S and CM columns and their equivalents and comparables.

In addition, antibodies described herein can be chemically synthesized using techniques known in the art (e.g., see Creighton, 1983, Proteins: Structures and Molecular Principles, W. H. Freeman & Co., N.Y and Hunkapiller et al., Nature, 310:105-111 (1984)). For example, a polypeptide corresponding to a fragment of a polypeptide can be synthesized by use of a peptide synthesizer. Furthermore, if desired, nonclassical amino acids or chemical amino acid analogs can be introduced as a substitution or addition into the polypeptide sequence. Nonclassical amino acids include, but are not limited to, to the D-isomers of the common amino acids, 2,4diaminobutyric acid, alpha-amino isobutyric acid, 4aminobutyric acid, Abu, 2-amino butyric acid, g-Abu, e-Ahx, 6amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, alanine, fluoro-amino acids, designer amino acids such as methyl amino acids, C-methyl amino acids, N-methyl amino acids, and amino acid analogs in general. Furthermore, the amino acid can be D (dextrorotary) or L (levorotary).

### Methods of Use

In an aspect, the present application provides methods of contacting OX40L with an anti-OX40L antibody, or antigen binding fragment thereof, such as a human or humanized antibody, which results in inhibition of OX40L binding to OX40 expressed on a cell.

In an aspect, the present application provides methods of using the isolated anti-OX40L antibodies, or antigen binding fragments thereof, described herein for treatment of a disorder or disease in a subject. In certain aspects, described herein is a method for treating a subject in need thereof with an anti-OX40L antibody, or antigen binding fragment thereof, the method comprising administering to the subject (e.g., a mammalian subject) a therapeutically effective amount of an anti-OX40L antibody, or antigen binding fragment thereof, or pharmaceutical composition comprising an anti-OX40L antibody, or antigen binding fragment thereof, described herein. In certain embodiments, the present application provides methods of treating a disorder or disease associated with elevated levels of OX40L and/or IgE in a subject.

In certain aspects, described herein are methods for treating a pathology associated with OX40L activity, the method comprising administering to a mammalian subject a therapeutically effective amount an isolated anti-OX40L antibody, or antigen binding fragment thereof, or a pharmaceutical composition comprising an isolated anti-OX40L antibody, or antigen binding fragment thereof, described herein.

In certain embodiments, the isolated antibody, or antigen binding fragment thereof, is used in the treatment of an inflammatory disorder or disease. In certain embodiments, the isolated antibody, or antigen binding fragment thereof, is used in the treatment of atopic dermatitis. In certain embodiments, the treatment reduces disease severity in a patient and wherein disease severity is assessed by an atopic dermatitis disease severity outcome measure. In certain embodiments, the isolated antibody, or antigen binding fragment thereof, is used in the treatment of idiopathic pulmonary fibrosis. In certain embodiments, the isolated antibody, or antigen binding fragment thereof, is used in the treatment of alopecia areata. In certain embodiments, the isolated antibody, or antigen binding fragment thereof, is used in the treatment of chronic sinusitis with nasal polyps. In certain embodiments, the isolated antibody, or antigen binding fragment thereof, is used in the treatment of Chronic Rhinosinusitis without Nasal Polyps (CRSsNP). In certain embodiments, the isolated antibody, or antigen binding fragment thereof, is used in the treatment of eosinophilic esophagitis (EoE). In certain embodiments, the isolated antibody, or antigen binding fragment thereof, is used in the treatment of an Eosinophilic gastrointestinal disorder or disease (EGID), such as Eosinophilic Gastritis (EoG), Eosinophilic Enteritis (EoN), Eosinophilic Colitis (EoC), or Eosinophilic Gastroenteritis (EGE). In certain embodiments, the isolated antibody, or antigen binding fragment thereof, is used in the treatment of Churg-Strauss syndrome/Eosinophilic granulomatosis with polyangiitis (EGPA). In certain embodiments, the isolated antibody, or antigen binding fragment thereof, is used in the treatment of Prurigo Nodularis (PN). In certain embodiments, the isolated antibody, or antigen binding fragment thereof, is used in the treatment of Chronic Spontaneous Urticaria (CSU). In certain embodiments, the isolated antibody, or antigen binding fragment thereof, is used in the treatment of Chronic Pruritis of Unknown Origin (CPUO). In certain embodiments, the isolated antibody, or antigen binding fragment thereof, is used in the treatment of Bullous Pemphigoid (BP). In certain embodiments, the isolated antibody, or antigen binding fragment thereof, is used in the treatment of Cold Inducible Urticaria (ColdU). In certain embodiments, the isolated antibody, or antigen binding fragment thereof, is used in the treatment of Allergic Fungal Rhinosinusitis (AFRS). In certain embodiments, the isolated antibody, or antigen binding fragment thereof, is used in the treatment of Allergic Bronchopulmonary Aspergillosis (ABPA). In certain embodiments, the isolated antibody, or antigen binding fragment thereof, is used in the treatment of Chronic Obstructive Pulmonary Disease (COPD). In certain embodiments, the isolated antibody, or antigen binding fragment thereof, is used in the treatment of inflammatory bowel disease, such as Crohn's disease or ulcerative colitis. In certain embodiments, the isolated antibody, or antigen binding fragment thereof, is used in the treatment of psoriasis. In certain embodiments, the isolated antibody, or antigen binding fragment thereof, is used in the treatment of lupus. In certain embodiments, the isolated antibody, or antigen binding fragment thereof, is used in the treatment of rheumatoid arthritis. In certain embodiments, the isolated antibody, or antigen binding fragment thereof, is used in the treatment of hidradenitis suppurativa. In certain embodiments, the isolated antibody, or antigen binding fragment thereof, is used in the treatment of celiac disease. In certain embodiments, the isolated antibody, or antigen binding fragment thereof, is used in the treatment of systemic sclerosis. In certain embodiments, the isolated antibody, or antigen binding fragment thereof, is used in the treatment of allergic rhinitis. In certain embodiments, the isolated antibody, or antigen binding fragment thereof, is used in the treatment of eosinophilic fasciitis. In certain embodiments, the isolated antibody, or antigen binding fragment thereof, is used in the treatment of scleromyxedema. In certain embodiments, the isolated antibody, or antigen binding fragment thereof, is used in the treatment of scleredema. In certain embodiments, the isolated antibody, or antigen binding fragment thereof, is used in the treatment of nephrogenic systemic fibrosis.

In certain aspects, described herein is a method for treating an inflammatory disorder or disease in a mammalian subject in need thereof, the method comprising administering to the mammalian subject a therapeutically effective amount an antibody, or antigen binding fragment thereof, described herein or a pharmaceutical composition described herein. In certain embodiments of the methods described herein, the inflammatory disorder or disease is atopic dermatitis. In certain embodiments, the inflammatory disorder or disease is asthma. In certain embodiments, the inflammatory disorder or disease is idiopathic pulmonary fibrosis. In certain embodiments of the methods described herein, the inflammatory disorder or disease is alopecia areata. In certain embodiments, the inflammatory disorder or disease is chronic sinusitis with nasal polyps. In certain embodiments, the inflammatory disorder or disease is Chronic Rhinosinusitis without Nasal Polyps (CRSsNP). In certain embodiments, the inflammatory disorder or disease is eosinophilic esophagitis (EoE). In certain embodiments, the inflammatory disorder or disease is an Eosinophilic gastrointestinal disorder or disease (EGID), such as Eosinophilic Gastritis (EoG), Eosinophilic Enteritis (EoN), Eosinophilic Colitis (EoC), or Eosinophilic Gastroenteritis (EGE). In certain embodiments, the inflammatory disorder or disease is Churg-Strauss syndrome/Eosinophilic granulomatosis with polyangiitis (EGPA). In certain embodiments, the inflammatory disorder or disease is Prurigo Nodularis (PN). In certain embodiments, the inflammatory disorder or disease is Chronic Spontaneous Urticaria (CSU). In certain embodiments, the inflammatory disorder or disease is Chronic Pruritis of Unknown Origin (CPUO). In certain embodiments, the inflammatory disorder or disease is Bullous Pemphigoid (BP). In certain embodiments, the inflammatory disorder or disease is Cold Inducible Urticaria (ColdU). In certain embodiments, the inflammatory disorder or disease is Allergic Fungal Rhinosinusitis (AFRS). In certain embodiments, the inflammatory disorder or disease is Allergic Bronchopulmonary Aspergillosis (ABPA). In certain embodiments, the inflammatory disorder or disease is Chronic Obstructive Pulmonary Disease (COPD). In certain embodiments, the inflammatory disorder or disease is inflammatory bowel disease, such as Crohn's disease or ulcerative colitis. In certain embodiments, the inflammatory disorder or disease is psoriasis. In certain embodiments, the inflammatory disorder or disease is lupus. In certain embodiments, the inflammatory disorder or disease is rheumatoid arthritis. In certain embodiments, the inflammatory disorder or disease is celiac disease. In certain embodiments, the inflammatory disorder or disease is hidradenitis suppurativa. In certain embodiments, the inflammatory disorder or disease is systemic sclerosis. In certain embodiments, the inflammatory disorder or disease is allergic rhinitis. In certain embodiments, the inflammatory disorder or disease is eosinophilic fasciitis. In certain embodiments, the inflammatory disorder or disease is scleromyxedema. In certain embodiments, the inflammatory disorder or disease is scleredema. In certain embodiments, the inflammatory disorder or disease is nephrogenic systemic fibrosis.

In certain aspects, described herein are methods for treating a pathology associated with elevated levels of OX40L in a mammalian subject in need thereof, the method comprising administering to the mammalian subject a therapeutically effective amount an antibody, or antigen binding fragment thereof, or a pharmaceutical composition described herein.

In certain aspects, described herein are methods of reducing biological activity of OX40L in a mammalian subject in need thereof, the method comprising administering to the mammalian subject a therapeutically effective amount an antibody, or antigen binding fragment thereof, or a pharmaceutical composition described herein.

In certain aspects, described herein are methods of preventing an inflammatory disorder or disease in a mammalian subject in need thereof, the method comprising administering to the mammalian subject a therapeutically effective amount an antibody, or antigen binding fragment thereof, or a pharmaceutical composition described herein.

Any of the above-described methods may further include administration of a hyaluronidase or a variant thereof (e.g., rHuPH20). In some embodiments, the hyaluronidase or variant thereof is included in the same composition as the anti-OX40L antibody, or antigen binding fragment thereof. In other embodiments, the hyaluronidase or variant thereof is administered separately from the anti-OX40L antibody, or antigen binding fragment thereof. The hyaluronidase or variant thereof may be administered before or after the anti-OX40L antibody, or antigen binding fragment thereof, or may be administered at about the same time as the anti-OX40L antibody or antigen binding fragment thereof (e.g., in a separate composition or via co-formulation).

### Methods of Administration

In some embodiments, the methods provided herein are useful for the treatment of a disease or disorder in an individual, e.g., a human. In an embodiment, the individual is a human and the antibody is an anti-OX40L antibody, or antigen binding fragment thereof, described herein.

In some embodiments, an antibody, or antigen binding fragment thereof, is administered intravenously, intramuscularly, subcutaneously, topically, orally, transdermally, intraperitoneally, intraorbitally, by implantation, by inhalation, intrathecally, intraventricularly, or intranasally. An effective amount of an anti-OX40L antibody, or antigen binding fragment thereof, may be administered for the treatment of a disease or disorder. The appropriate dosage of the anti-OX40L antibody, or antigen binding fragment thereof, may be determined based on the type of disease or disorder to be treated, the type of the anti-OX40L antibody, the severity and course of the disease or disorder, the clinical condition of the individual, the individual's clinical history and response to the treatment, and the discretion of the attending physician.

In some embodiments, an antibody, or antigen binding fragment thereof, provided herein is administered with at least one additional therapeutic agent. Any suitable additional therapeutic or immunotherapeutic agent may be administered with an antibody, or antigen binding fragment thereof, provided herein. Additional therapeutic agents include agents that are used to treat or prevent a disease or disorder such as, but not limited to, an inflammatory disease or disorder associated with elevated levels of OX40L and/or IgE.

The additional therapeutic agent can be administered by any suitable means. In some embodiments, an antibody, or antigen binding fragment thereof, provided herein and the additional therapeutic agent are included in the same pharmaceutical composition. In some embodiments, an antibody, or antigen binding fragment thereof, provided herein and the additional therapeutic agent are included in different pharmaceutical compositions.

In embodiments where an antibody, or antigen binding fragment thereof, provided herein and the additional therapeutic agent are included in different pharmaceutical compositions, administration of the antibody, or antigen binding fragment thereof, can occur prior to, simultaneously, and/or following, administration of the additional therapeutic agent. In some embodiments, administration of an antibody, or antigen binding fragment thereof, provided herein and the additional therapeutic agent occur within about one month of each other. In some embodiments, administration of an antibody, or antigen binding fragment thereof, provided herein and the additional therapeutic agent occur within about one week of each other. In some embodiments, administration of an antibody, or antigen binding fragment thereof, provided herein and the additional therapeutic agent occur within about one day of each other. In some embodiments, administration of an antibody, or antigen binding fragment thereof, provided herein and the additional therapeutic agent occur within about twelve hours of each other. In some embodiments, administration of an antibody, or antigen binding fragment thereof, provided herein and the additional therapeutic agent occur within about one hour of each other.

### Compositions, Combinations, and Methods Involving Co-Administration

In certain aspects, provided herein are compositions comprising any of the antibodies, or antigen binding fragments thereof, that bind OX40L described herein and hyaluronidase or variant thereof.

In certain aspects, described herein are a combination of any one of the antibodies, or antigen binding fragments thereof, that bind OX40L described herein and a hyaluronidase or variant thereof.

In certain aspects, described herein are methods of treating an inflammatory disorder or disease in a human patient comprising co-administering to the patient any one of the antibodies, or antigen binding fragments thereof, that bind OX40L described herein and a hyaluronidase or variant thereof.

In certain embodiments, the antibody, or antigen binding fragment thereof, is a humanized, human, or chimeric antibody. In certain embodiments, the antibody, or antigen binding fragment thereof, is a monoclonal antibody. In certain embodiments, the antibody, or antigen binding fragment thereof, comprises a heavy chain human constant region of a class selected from IgG, IgA, IgD, IgE, and IgM. In certain embodiments, the antibody, or antigen binding fragment thereof, comprises a human Fc region comprising a human heavy chain constant region of the class IgG and a subclass selected from IgG1, IgG2, IgG3, and IgG4. In certain embodiments, the human Fc region comprises a human IgG1 Fc region.

In certain embodiments the human Fc region comprises a human IgG1 Fc region with LALA mutations. In certain embodiments the human Fc region comprises a human IgG1 Fc region with YTE mutations. In certain embodiments the human Fc region comprises a human IgG1 Fc region with LALA and YTE mutations. In certain embodiments, when direct numbering is used these “YTE” and “LALA” mutations can be located at different amino acid position numbers. For example, in one embodiment, the human Fc region comprises a human IgG1 Fc region with LALA mutations at L235A/L236A and/or YTE mutations at M253Y/S255T/T257E.

In certain embodiments, the antibody, or antigen binding fragment thereof, that binds OX40L comprises a CDR-H1 comprising the sequence set forth in SEQ ID NO:1; a CDR-H2 comprising the sequence set forth in SEQ ID NO: 70; a CDR-H3 comprising the sequence set forth in SEQ ID NO: 175; a CDR-L1 comprising the sequence set forth in SEQ ID NO: 258; a CDR-L2 comprising the sequence set forth in SEQ ID NO: 335; and a CDR-L3 comprising the sequence set forth in SEQ ID NO: 368. In certain embodiments, the antibody, or antigen binding fragment thereof, that binds OX40L comprises a VH sequence set forth in SEQ ID NO: 419 and a VL sequence set forth in SEQ ID NO: 489. In certain embodiments, the antibody, or antigen binding fragment thereof, comprises a heavy chain comprising a constant heavy chain sequence set forth in any one of SEQ ID NOs: 637-737 and 913-1012. In certain embodiments, the antibody, or antigen binding fragment thereof, comprises a heavy chain comprising a constant heavy chain sequence set forth in SEQ ID NO: 651 or SEQ ID NO: 927. In certain embodiments, the antibody, or antigen binding fragment thereof, comprises a heavy chain comprising a constant heavy chain sequence set forth in SEQ ID NO: 651. In certain embodiments, the antibody, or antigen binding fragment thereof, comprises a heavy chain comprising a constant heavy chain sequence set forth in SEQ ID NO: 927. In certain embodiments, the antibody, or antigen binding fragment thereof, comprises a light chain comprising a constant light chain sequence set forth in SEQ ID NO: 738. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 927 and/or a constant heavy chain sequence set forth in SEQ ID NO: 651, and a constant light chain sequence set forth in SEQ ID NO: 738. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 927, and a constant light chain sequence set forth in SEQ ID NO: 738. In certain embodiments, the antibody, or antigen binding fragment thereof, comprises a heavy chain sequence set forth in SEQ ID NO: 1013 and a light chain sequence set forth in SEQ ID NO: 1015. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 651, and a constant light chain sequence set forth in SEQ ID NO: 738. In certain embodiments, the antibody, or antigen binding fragment thereof, comprises a heavy chain sequence set forth in SEQ ID NO: 1014 and a light chain sequence set forth in SEQ ID NO: 1015.

In certain embodiments, the antibody, or antigen binding fragment thereof, that binds OX40L comprises a CDR-H1 comprising the sequence set forth in SEQ ID NO: 44; a CDR-H2 comprising the sequence set forth in SEQ ID NO: 910; a CDR-H3 comprising the sequence set forth in SEQ ID NO: 911; a CDR-L1 comprising the sequence set forth in SEQ ID NO: 334; a CDR-L2 comprising the sequence set forth in SEQ ID NO: 361; and a CDR-L3 comprising the sequence set forth in SEQ ID NO: 912. In certain embodiments, the antibody, or antigen binding fragment thereof, that binds OX40L comprises a VH sequence set forth in SEQ ID NO: 832 and a VL sequence set forth in SEQ ID NO: 833. In certain embodiments, the antibody, or antigen binding fragment thereof comprises a heavy chain comprising a constant heavy chain sequence selected from a sequence set forth in any one of SEQ ID NOs: 637-737 and 913-1012. In certain embodiments, the antibody has a heavy chain sequence set forth in SEQ ID NO: 1018 and a light chain sequence set forth in SEQ ID NO: 1019. In certain embodiments, the antibody, or antigen binding fragment thereof comprises a heavy chain comprising a constant heavy chain sequence selected from a sequence set forth in any one of SEQ ID NOs: 637-639, 641-737, 913-915, and 917-1012. In certain embodiments, the antibody, or antigen binding fragment thereof does not comprise a constant heavy chain sequence set forth in SEQ ID NOs: 640 and 916 (i.e., a human IgG4-SPLE constant heavy chain region).

In certain embodiments, the Fc region of the antibody, or antigen binding fragment thereof, binds to Neonatal Fc receptor (FcRn). In certain embodiments, the Fc region binds an FcRn with higher affinity at pH 6.0 compared to an antibody comprising a wild-type Fc region. In certain embodiments, the Fc region binds to FcRn with a K_{D} of <1×10⁻⁷ M at pH 6.0. In certain embodiments, the antibody, or antigen binding fragment thereof, binds an OX40L sequence set forth in SEQ ID NO: 739.

Any suitable hyaluronidase or variant thereof can be used in the compositions, combinations, and methods described herein. In certain embodiments, the hyaluronidase is a soluble hyaluronidase. In certain embodiments, the hyaluronidase is a recombinant human hyaluronidase. In certain embodiments, the recombinant human hyaluronidase comprises the amino acid sequence set forth in any one of SEQ ID NOs: 843-852 and 854-909, or a mixture thereof (e.g., the recombinant human hyaluronidase may comprise one or more of SEQ ID NOs: 843-852 and 854-909). In certain embodiments, the recombinant human hyaluronidase is rHuPH20 (i.e., a composition comprising one or more of SEQ ID NOs: 845-850). In certain embodiments, the rHuPH20 formulation is ENHANZE®.

In one embodiment, the recombinant human hyaluronidase includes a sequence of amino acids in any one of SEQ ID NOs: 843-852 and 854-909, or has at least 80%, 81%, 82%, 83%, 84%, 85%, 86%, 87%, 88%, 89%, 90%, 91%, 92%, 93%, 94%, 95%, 96%, 97%, 98%, or 99% sequence identity to a sequence of amino acids included in any one of SEQ ID NOs: 843-852 and 854-909 and retains hyaluronidase activity. In one embodiment, the recombinant human hyaluronidase comprises a sequence having at least 95%, 96%, 97%, 98%, or 99% to the amino acid sequence of SEQ ID NO: 843. In one embodiment, the recombinant human hyaluronidase comprises a sequence having at least 95%, 96%, 97%, 98%, or 99% to the amino acid sequence of SEQ ID NO: 845. In another embodiment, the recombinant human hyaluronidase comprises the amino acid sequence set forth in SEQ ID NO:843. In another embodiment, the recombinant human hyaluronidase consists of the amino acid sequence set forth in SEQ ID NO:843. In another embodiment, the recombinant human hyaluronidase comprises the amino acid sequence set forth in SEQ ID NO:844. In another embodiment, the recombinant human hyaluronidase consists of the amino acid sequence set forth in SEQ ID NO:844. In another embodiment, the recombinant human hyaluronidase comprises the amino acid sequence set forth in SEQ ID NO:845. In another embodiment, the recombinant human hyaluronidase consists of the amino acid sequence set forth in SEQ ID NO:845. In another embodiment, the recombinant human hyaluronidase comprises the amino acid sequence set forth in SEQ ID NO:846. In another embodiment, the recombinant human hyaluronidase consists of the amino acid sequence set forth in SEQ ID NO:846. In another embodiment, the recombinant human hyaluronidase comprises the amino acid sequence set forth in SEQ ID NO:847. In another embodiment, the recombinant human hyaluronidase consists of the amino acid sequence set forth in SEQ ID NO:847. In another embodiment, the recombinant human hyaluronidase comprises the amino acid sequence set forth in SEQ ID NO:848. In another embodiment, the recombinant human hyaluronidase consists of the amino acid sequence set forth in SEQ ID NO:848. In another embodiment, the recombinant human hyaluronidase comprises the amino acid sequence set forth in SEQ ID NO:849. In another embodiment, the recombinant human hyaluronidase consists of the amino acid sequence set forth in SEQ ID NO:849. In another embodiment, the recombinant human hyaluronidase comprises the amino acid sequence set forth in SEQ ID NO:850. In another embodiment, the recombinant human hyaluronidase consists of the amino acid sequence set forth in SEQ ID NO:850. In another embodiment, the recombinant human hyaluronidase comprises the amino acid sequence set forth in SEQ ID NO:851. In another embodiment, the recombinant human hyaluronidase consists of the amino acid sequence set forth in SEQ ID NO:851. In another embodiment, the recombinant human hyaluronidase comprises the amino acid sequence set forth in SEQ ID NO:852. In another embodiment, the recombinant human hyaluronidase consists of the amino acid sequence set forth in SEQ ID NO:852. In another embodiment, the recombinant human hyaluronidase comprises the amino acid sequence set forth in SEQ ID NO:854. In another embodiment, the recombinant human hyaluronidase consists of the amino acid sequence set forth in SEQ ID NO:854. In another embodiment, the recombinant human hyaluronidase comprises the amino acid sequence set forth in SEQ ID NO:855. In another embodiment, the recombinant human hyaluronidase consists of the amino acid sequence set forth in SEQ ID NO:855. In another embodiment, the recombinant human hyaluronidase comprises the amino acid sequence set forth in SEQ ID NO:856. In another embodiment, the recombinant human hyaluronidase consists of the amino acid sequence set forth in SEQ ID NO:856. In another embodiment, the recombinant human hyaluronidase comprises the amino acid sequence set forth in SEQ ID NO:857. In another embodiment, the recombinant human hyaluronidase consists of the amino acid sequence set forth in SEQ ID NO:857. In another embodiment, the recombinant human hyaluronidase comprises the amino acid sequence set forth in SEQ ID NO:858. In another embodiment, the recombinant human hyaluronidase consists of the amino acid sequence set forth in SEQ ID NO:858. In another embodiment, the recombinant human hyaluronidase comprises the amino acid sequence set forth in SEQ ID NO:859. In another embodiment, the recombinant human hyaluronidase consists of the amino acid sequence set forth in SEQ ID NO:859. In another embodiment, the recombinant human hyaluronidase comprises the amino acid sequence set forth in SEQ ID NO:860. In another embodiment, the recombinant human hyaluronidase consists of the amino acid sequence set forth in SEQ ID NO:860. In another embodiment, the recombinant human hyaluronidase comprises the amino acid sequence set forth in SEQ ID NO:861. In another embodiment, the recombinant human hyaluronidase consists of the amino acid sequence set forth in SEQ ID NO:861. In another embodiment, the recombinant human hyaluronidase comprises the amino acid sequence set forth in SEQ ID NO:862. In another embodiment, the recombinant human hyaluronidase consists of the amino acid sequence set forth in SEQ ID NO:862. In another embodiment, the recombinant human hyaluronidase comprises the amino acid sequence set forth in SEQ ID NO:863. In another embodiment, the recombinant human hyaluronidase consists of the amino acid sequence set forth in SEQ ID NO:863. In another embodiment, the recombinant human hyaluronidase comprises the amino acid sequence set forth in SEQ ID NO:864. In another embodiment, the recombinant human hyaluronidase consists of the amino acid sequence set forth in SEQ ID NO:864. In another embodiment, the recombinant human hyaluronidase comprises the amino acid sequence set forth in SEQ ID NO:865. In another embodiment, the recombinant human hyaluronidase consists of the amino acid sequence set forth in SEQ ID NO:865. In another embodiment, the recombinant human hyaluronidase comprises the amino acid sequence set forth in SEQ ID NO:866. In another embodiment, the recombinant human hyaluronidase consists of the amino acid sequence set forth in SEQ ID NO:866. In another embodiment, the recombinant human hyaluronidase comprises the amino acid sequence set forth in SEQ ID NO:867. In another embodiment, the recombinant human hyaluronidase consists of the amino acid sequence set forth in SEQ ID NO:867. In another embodiment, the recombinant human hyaluronidase comprises the amino acid sequence set forth in SEQ ID NO:868. In another embodiment, the recombinant human hyaluronidase consists of the amino acid sequence set forth in SEQ ID NO:868. In another embodiment, the recombinant human hyaluronidase comprises the amino acid sequence set forth in SEQ ID NO:869. In another embodiment, the recombinant human hyaluronidase consists of the amino acid sequence set forth in SEQ ID NO:869. In another embodiment, the recombinant human hyaluronidase comprises the amino acid sequence set forth in SEQ ID NO:870. In another embodiment, the recombinant human hyaluronidase consists of the amino acid sequence set forth in SEQ ID NO:870. In another embodiment, the recombinant human hyaluronidase comprises the amino acid sequence set forth in SEQ ID NO:871. In another embodiment, the recombinant human hyaluronidase consists of the amino acid sequence set forth in SEQ ID NO:871. In another embodiment, the recombinant human hyaluronidase comprises the amino acid sequence set forth in SEQ ID NO:872. In another embodiment, the recombinant human hyaluronidase consists of the amino acid sequence set forth in SEQ ID NO:872. In another embodiment, the recombinant human hyaluronidase comprises the amino acid sequence set forth in SEQ ID NO:873. In another embodiment, the recombinant human hyaluronidase consists of the amino acid sequence set forth in SEQ ID NO:873. In another embodiment, the recombinant human hyaluronidase comprises the amino acid sequence set forth in SEQ ID NO:874. In another embodiment, the recombinant human hyaluronidase consists of the amino acid sequence set forth in SEQ ID NO:874. In another embodiment, the recombinant human hyaluronidase comprises the amino acid sequence set forth in SEQ ID NO:875. In another embodiment, the recombinant human hyaluronidase consists of the amino acid sequence set forth in SEQ ID NO:875. In another embodiment, the recombinant human hyaluronidase comprises the amino acid sequence set forth in SEQ ID NO:876. In another embodiment, the recombinant human hyaluronidase consists of the amino acid sequence set forth in SEQ ID NO:876. In another embodiment, the recombinant human hyaluronidase comprises the amino acid sequence set forth in SEQ ID NO:877. In another embodiment, the recombinant human hyaluronidase consists of the amino acid sequence set forth in SEQ ID NO:877. In another embodiment, the recombinant human hyaluronidase comprises the amino acid sequence set forth in SEQ ID NO:878. In another embodiment, the recombinant human hyaluronidase consists of the amino acid sequence set forth in SEQ ID NO:878. In another embodiment, the recombinant human hyaluronidase comprises the amino acid sequence set forth in SEQ ID NO:879. In another embodiment, the recombinant human hyaluronidase consists of the amino acid sequence set forth in SEQ ID NO:879. In another embodiment, the recombinant human hyaluronidase comprises the amino acid sequence set forth in SEQ ID NO:880. In another embodiment, the recombinant human hyaluronidase consists of the amino acid sequence set forth in SEQ ID NO:880. In another embodiment, the recombinant human hyaluronidase comprises the amino acid sequence set forth in SEQ ID NO:881. In another embodiment, the recombinant human hyaluronidase consists of the amino acid sequence set forth in SEQ ID NO:881. In another embodiment, the recombinant human hyaluronidase comprises the amino acid sequence set forth in SEQ ID NO:882. In another embodiment, the recombinant human hyaluronidase consists of the amino acid sequence set forth in SEQ ID NO:882. In another embodiment, the recombinant human hyaluronidase comprises the amino acid sequence set forth in SEQ ID NO:883. In another embodiment, the recombinant human hyaluronidase consists of the amino acid sequence set forth in SEQ ID NO:883. In another embodiment, the recombinant human hyaluronidase comprises the amino acid sequence set forth in SEQ ID NO:884. In another embodiment, the recombinant human hyaluronidase consists of the amino acid sequence set forth in SEQ ID NO:884. In another embodiment, the recombinant human hyaluronidase comprises the amino acid sequence set forth in SEQ ID NO:885. In another embodiment, the recombinant human hyaluronidase consists of the amino acid sequence set forth in SEQ ID NO:885. In another embodiment, the recombinant human hyaluronidase comprises the amino acid sequence set forth in SEQ ID NO:886. In another embodiment, the recombinant human hyaluronidase consists of the amino acid sequence set forth in SEQ ID NO:886. In another embodiment, the recombinant human hyaluronidase comprises the amino acid sequence set forth in SEQ ID NO:887. In another embodiment, the recombinant human hyaluronidase consists of the amino acid sequence set forth in SEQ ID NO:887. In another embodiment, the recombinant human hyaluronidase comprises the amino acid sequence set forth in SEQ ID NO:888. In another embodiment, the recombinant human hyaluronidase consists of the amino acid sequence set forth in SEQ ID NO:888. In another embodiment, the recombinant human hyaluronidase comprises the amino acid sequence set forth in SEQ ID NO:889. In another embodiment, the recombinant human hyaluronidase consists of the amino acid sequence set forth in SEQ ID NO:889. In another embodiment, the recombinant human hyaluronidase comprises the amino acid sequence set forth in SEQ ID NO:890. In another embodiment, the recombinant human hyaluronidase consists of the amino acid sequence set forth in SEQ ID NO:890. In another embodiment, the recombinant human hyaluronidase comprises the amino acid sequence set forth in SEQ ID NO:891. In another embodiment, the recombinant human hyaluronidase consists of the amino acid sequence set forth in SEQ ID NO:891. In another embodiment, the recombinant human hyaluronidase comprises the amino acid sequence set forth in SEQ ID NO:892. In another embodiment, the recombinant human hyaluronidase consists of the amino acid sequence set forth in SEQ ID NO:892. In another embodiment, the recombinant human hyaluronidase comprises the amino acid sequence set forth in SEQ ID NO:893. In another embodiment, the recombinant human hyaluronidase consists of the amino acid sequence set forth in SEQ ID NO:893. In another embodiment, the recombinant human hyaluronidase comprises the amino acid sequence set forth in SEQ ID NO:894. In another embodiment, the recombinant human hyaluronidase consists of the amino acid sequence set forth in SEQ ID NO:894. In another embodiment, the recombinant human hyaluronidase comprises the amino acid sequence set forth in SEQ ID NO:895. In another embodiment, the recombinant human hyaluronidase consists of the amino acid sequence set forth in SEQ ID NO:895. In another embodiment, the recombinant human hyaluronidase comprises the amino acid sequence set forth in SEQ ID NO:896. In another embodiment, the recombinant human hyaluronidase consists of the amino acid sequence set forth in SEQ ID NO:896. In another embodiment, the recombinant human hyaluronidase comprises the amino acid sequence set forth in SEQ ID NO:897. In another embodiment, the recombinant human hyaluronidase consists of the amino acid sequence set forth in SEQ ID NO:897. In another embodiment, the recombinant human hyaluronidase comprises the amino acid sequence set forth in SEQ ID NO:898. In another embodiment, the recombinant human hyaluronidase consists of the amino acid sequence set forth in SEQ ID NO:898. In another embodiment, the recombinant human hyaluronidase comprises the amino acid sequence set forth in SEQ ID NO:899. In another embodiment, the recombinant human hyaluronidase consists of the amino acid sequence set forth in SEQ ID NO:899. In another embodiment, the recombinant human hyaluronidase comprises the amino acid sequence set forth in SEQ ID NO:900. In another embodiment, the recombinant human hyaluronidase consists of the amino acid sequence set forth in SEQ ID NO:900. In another embodiment, the recombinant human hyaluronidase comprises the amino acid sequence set forth in SEQ ID NO:901. In another embodiment, the recombinant human hyaluronidase consists of the amino acid sequence set forth in SEQ ID NO:901. In another embodiment, the recombinant human hyaluronidase comprises the amino acid sequence set forth in SEQ ID NO:902. In another embodiment, the recombinant human hyaluronidase consists of the amino acid sequence set forth in SEQ ID NO:902. In another embodiment, the recombinant human hyaluronidase comprises the amino acid sequence set forth in SEQ ID NO:903. In another embodiment, the recombinant human hyaluronidase consists of the amino acid sequence set forth in SEQ ID NO:903. In another embodiment, the recombinant human hyaluronidase comprises the amino acid sequence set forth in SEQ ID NO:904. In another embodiment, the recombinant human hyaluronidase consists of the amino acid sequence set forth in SEQ ID NO:904. In another embodiment, the recombinant human hyaluronidase comprises the amino acid sequence set forth in SEQ ID NO:905. In another embodiment, the recombinant human hyaluronidase consists of the amino acid sequence set forth in SEQ ID NO:905. In another embodiment, the recombinant human hyaluronidase comprises the amino acid sequence set forth in SEQ ID NO:906. In another embodiment, the recombinant human hyaluronidase consists of the amino acid sequence set forth in SEQ ID NO:906. In another embodiment, the recombinant human hyaluronidase comprises the amino acid sequence set forth in SEQ ID NO:907. In another embodiment, the recombinant human hyaluronidase consists of the amino acid sequence set forth in SEQ ID NO:907. In another embodiment, the recombinant human hyaluronidase comprises the amino acid sequence set forth in SEQ ID NO:908. In another embodiment, the recombinant human hyaluronidase consists of the amino acid sequence set forth in SEQ ID NO:908. In another embodiment, the recombinant human hyaluronidase comprises the amino acid sequence set forth in SEQ ID NO:909. In another embodiment, the recombinant human hyaluronidase consists of the amino acid sequence set forth in SEQ ID NO:909.

In certain embodiments, the hyaluronidase or variant thereof and the OX40L antibody, or antigen binding fragment thereof, are administered in separate formulations. In certain embodiments, the hyaluronidase or variant thereof and the anti-OX40L antibody, or antigen binding fragment thereof, are administered simultaneously in separate formulations. In certain embodiments, the hyaluronidase or variant thereof is administered to the patient prior to administration of the anti-OX40L antibody, or antigen binding fragment thereof. In certain embodiments, the hyaluronidase or variant thereof is administered to the patient after administration of the anti-OX40L antibody, or antigen binding fragment thereof. In certain embodiments, the hyaluronidase or variant thereof and the anti-OX40L antibody, or antigen binding fragment thereof, are mixed and administered in a single formulation. In certain embodiments, the hyaluronidase or variant thereof and the anti-OX40L antibody, or antigen binding fragment thereof, are administered by subcutaneous injection. In certain embodiments, the hyaluronidase or variant thereof and the anti-OX40L antibody, or antigen binding fragment thereof, are administered by intravenous injection.

In certain embodiments, the hyaluronidase is rHuPH20 administered at a concentration of 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, or 40,000 units.

In certain embodiments, methods of treating an inflammatory disorder or disease in a human patient are provided, wherein the method comprises co-administering to the patient any one of the antibodies, or antigen binding fragments thereof, that bind OX40L described herein and a hyaluronidase or variant thereof. In certain embodiments, the inflammatory disorder or disease is atopic dermatitis. In certain embodiments, the treatment reduces disease severity in the patient and wherein disease severity is assessed by an atopic dermatitis disease severity outcome measure. In certain embodiments, the inflammatory disorder or disease is asthma, idiopathic pulmonary fibrosis, alopecia areata, chronic sinusitis with nasal polyps, Chronic Rhinosinusitis without Nasal Polyps (CRSsNP), eosinophilic esophagitis (EoE), Churg-Strauss syndrome/Eosinophilic granulomatosis with polyangiitis (EGPA), Prurigo Nodularis (PN), Chronic Spontaneous Urticaria (CSU), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), lupus, rheumatoid arthritis (RA), psoriasis, an inflammatory bowel disease, such as Crohn's disease or ulcerative colitis, hidradenitis suppurativa, celiac disease, systemic sclerosis, allergic rhinitis, eosinophilic fasciitis, scleromyxedema, scleredema, or nephrogenic systemic fibrosis. In certain embodiments, the inflammatory disorder or disease is an Eosinophilic gastrointestinal disorder or disease (EGID), such as Eosinophilic Gastritis (EoG), Eosinophilic Enteritis (EoN), Eosinophilic Colitis (EoC), or Eosinophilic Gastroenteritis (EGE).

In certain aspects, described herein are compositions comprising an antibody, or antigen binding fragment thereof, that binds OX40L wherein the antibody, or antigen binding fragment thereof, comprises a CDR-H1 comprising the sequence set forth in SEQ ID NO:1; a CDR-H2 comprising the sequence set forth in SEQ ID NO: 70; a CDR-H3 comprising the sequence set forth in SEQ ID NO: 175; a CDR-L1 comprising the sequence set forth in SEQ ID NO: 258; a CDR-L2 comprising the sequence set forth in SEQ ID NO: 335; and a CDR-L3 comprising the sequence set forth in SEQ ID NO: 368, and a hyaluronidase or variant thereof. In certain embodiments, the antibody, or antigen binding fragment thereof, that binds OX40L comprises a VH sequence set forth in SEQ ID NO: 419 and a VL sequence set forth in SEQ ID NO: 489. In certain embodiments, the antibody, or antigen binding fragment thereof, comprises a heavy chain comprising a constant heavy chain sequence set forth in any one of SEQ ID NOs: 637-737 and 913-1012. In certain embodiments, the antibody, or antigen binding fragment thereof, comprises a heavy chain comprising a constant heavy chain sequence set forth in SEQ ID NO: 651 or SEQ ID NO: 927. In certain embodiments, the antibody, or antigen binding fragment thereof, comprises a heavy chain comprising a constant heavy chain sequence set forth in SEQ ID NO: 651. In certain embodiments, the antibody, or antigen binding fragment thereof, comprises a heavy chain comprising a constant heavy chain sequence set forth in SEQ ID NO: 927. In certain embodiments, the antibody, or antigen binding fragment thereof, comprises a light chain comprising a constant light chain sequence set forth in SEQ ID NO: 738. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 927 and/or a constant heavy chain sequence set forth in SEQ ID NO: 651, and a constant light chain sequence set forth in SEQ ID NO: 738. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 927, and a constant light chain sequence set forth in SEQ ID NO: 738. In certain embodiments, the antibody, or antigen binding fragment thereof, comprises a heavy chain sequence set forth in SEQ ID NO: 1013 and a light chain sequence set forth in SEQ ID NO: 1015. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 651, and a constant light chain sequence set forth in SEQ ID NO: 738. In certain embodiments, the antibody, or antigen binding fragment thereof, comprises a heavy chain sequence set forth in SEQ ID NO: 1014 and a light chain sequence set forth in SEQ ID NO: 1015.

In certain aspects, described herein are compositions comprising an antibody, or antigen binding fragment thereof, that binds OX40L wherein the antibody, or antigen binding fragment thereof, comprises a CDR-H1 comprising the sequence set forth in SEQ ID NO: 44; a CDR-H2 comprising the sequence set forth in SEQ ID NO: 910; a CDR-H3 comprising the sequence set forth in SEQ ID NO: 911; a CDR-L1 comprising the sequence set forth in SEQ ID NO: 334; a CDR-L2 comprising the sequence set forth in SEQ ID NO: 361; and a CDR-L3 comprising the sequence set forth in SEQ ID NO: 912, and a hyaluronidase or variant thereof. In certain embodiments, the antibody, or antigen binding fragment thereof, that binds OX40L comprises a VH sequence set forth in SEQ ID NO: 832 and a VL sequence set forth in SEQ ID NO: 833. In certain embodiments, the antibody, or antigen binding fragment thereof comprises a heavy chain comprising a constant heavy chain sequence selected from a sequence set forth in any one of SEQ ID NOs: 637-737 and 913-1012. In certain embodiments, the antibody has a heavy chain sequence set forth in SEQ ID NO: 1018 and a light chain sequence set forth in SEQ ID NO: 1019. In certain embodiments, the antibody, or antigen binding fragment thereof comprises a heavy chain comprising a constant heavy chain sequence selected from a sequence set forth in any one of SEQ ID NOs: 637-639, 641-737, 913-915, and 917-1012. In certain embodiments, the antibody, or antigen binding fragment thereof does not comprise a constant heavy chain sequence set forth in SEQ ID NOs: 640 and 916 (i.e., a human IgG4-SPLE constant heavy chain region).

In certain aspects, described herein are a combination of an antibody, or antigen binding fragment thereof, that binds OX40L, wherein the antibody, or antigen binding fragment thereof, comprises a CDR-H1 comprising the sequence set forth in SEQ ID NO:1; a CDR-H2 comprising the sequence set forth in SEQ ID NO: 70; a CDR-H3 comprising the sequence set forth in SEQ ID NO: 175; a CDR-L1 comprising the sequence set forth in SEQ ID NO: 258; a CDR-L2 comprising the sequence set forth in SEQ ID NO: 335; and a CDR-L3 comprising the sequence set forth in SEQ ID NO: 368, and a hyaluronidase or variant thereof. In certain embodiments, the antibody, or antigen binding fragment thereof, that binds OX40L comprises a VH sequence set forth in SEQ ID NO: 419 and a VL sequence set forth in SEQ ID NO: 489. In certain embodiments, the antibody, or antigen binding fragment thereof, comprises a heavy chain comprising a constant heavy chain sequence set forth in any one of SEQ ID NOs: 637-737 and 913-1012. In certain embodiments, the antibody, or antigen binding fragment thereof, comprises a heavy chain comprising a constant heavy chain sequence set forth in SEQ ID NO: 651 or SEQ ID NO: 927. In certain embodiments, the antibody, or antigen binding fragment thereof, comprises a heavy chain comprising a constant heavy chain sequence set forth in SEQ ID NO: 651. In certain embodiments, the antibody, or antigen binding fragment thereof, comprises a heavy chain comprising a constant heavy chain sequence set forth in SEQ ID NO: 927. In certain embodiments, the antibody, or antigen binding fragment thereof, comprises a light chain comprising a constant light chain sequence set forth in SEQ ID NO: 738. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 927 and/or a constant heavy chain sequence set forth in SEQ ID NO: 651, and a constant light chain sequence set forth in SEQ ID NO: 738. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 927, and a constant light chain sequence set forth in SEQ ID NO: 738. In certain embodiments, the antibody, or antigen binding fragment thereof, comprises a heavy chain sequence set forth in SEQ ID NO: 1013 and a light chain sequence set forth in SEQ ID NO: 1015. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 651, and a constant light chain sequence set forth in SEQ ID NO: 738. In certain embodiments, the antibody, or antigen binding fragment thereof, comprises a heavy chain sequence set forth in SEQ ID NO: 1014 and a light chain sequence set forth in SEQ ID NO: 1015.

In certain aspects, described herein are a combination of an antibody, or antigen binding fragment thereof, that binds OX40L, wherein the antibody, or antigen binding fragment thereof, comprises a CDR-H1 comprising the sequence set forth in SEQ ID NO: 44; a CDR-H2 comprising the sequence set forth in SEQ ID NO: 910; a CDR-H3 comprising the sequence set forth in SEQ ID NO: 911; a CDR-L1 comprising the sequence set forth in SEQ ID NO: 334; a CDR-L2 comprising the sequence set forth in SEQ ID NO: 361; and a CDR-L3 comprising the sequence set forth in SEQ ID NO: 912, and a hyaluronidase or variant thereof. In certain embodiments, the antibody, or antigen binding fragment thereof, that binds OX40L comprises a VH sequence set forth in SEQ ID NO: 832 and a VL sequence set forth in SEQ ID NO: 833. In certain embodiments, the antibody, or antigen binding fragment thereof comprises a heavy chain comprising a constant heavy chain sequence selected from a sequence set forth in any one of SEQ ID NOs: 637-737 and 913-1012. In certain embodiments, the antibody has a heavy chain sequence set forth in SEQ ID NO: 1018 and a light chain sequence set forth in SEQ ID NO: 1019. In certain embodiments, the antibody, or antigen binding fragment thereof comprises a heavy chain comprising a constant heavy chain sequence selected from a sequence set forth in any one of SEQ ID NOs: 637-639, 641-737, 913-915, and 917-1012. In certain embodiments, the antibody, or antigen binding fragment thereof does not comprise a constant heavy chain sequence set forth in SEQ ID NOs: 640 and 916 (i.e., a human IgG4-SPLE constant heavy chain region).

In certain aspects, described herein are methods of treating an inflammatory disorder or disease in a human patient comprising co-administering to the patient an antibody, or antigen binding fragment thereof, that binds OX40L, wherein the antibody, or antigen binding fragment thereof, comprises a CDR-H1 comprising the sequence set forth in SEQ ID NO:1; a CDR-H2 comprising the sequence set forth in SEQ ID NO: 70; a CDR-H3 comprising the sequence set forth in SEQ ID NO: 175; a CDR-L1 comprising the sequence set forth in SEQ ID NO: 258; a CDR-L2 comprising the sequence set forth in SEQ ID NO: 335; and a CDR-L3 comprising the sequence set forth in SEQ ID NO: 368, and a hyaluronidase or variant thereof. In certain embodiments, the antibody, or antigen binding fragment thereof, that binds OX40L comprises a VH sequence set forth in SEQ ID NO: 419 and a VL sequence set forth in SEQ ID NO: 489. In certain embodiments, the antibody, or antigen binding fragment thereof, comprises a heavy chain comprising a constant heavy chain sequence set forth in any one of SEQ ID NOs: 637-737 and 913-1012. In certain embodiments, the antibody, or antigen binding fragment thereof, comprises a heavy chain comprising a constant heavy chain sequence set forth in SEQ ID NO: 651 or SEQ ID NO: 927. In certain embodiments, the antibody, or antigen binding fragment thereof, comprises a heavy chain comprising a constant heavy chain sequence set forth in SEQ ID NO: 651. In certain embodiments, the antibody, or antigen binding fragment thereof, comprises a heavy chain comprising a constant heavy chain sequence set forth in SEQ ID NO: 927. In certain embodiments, the antibody, or antigen binding fragment thereof, comprises a light chain comprising a constant light chain sequence set forth in SEQ ID NO: 738. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 927 and/or a constant heavy chain sequence set forth in SEQ ID NO: 651, and a constant light chain sequence set forth in SEQ ID NO: 738. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 927, and a constant light chain sequence set forth in SEQ ID NO: 738. In certain embodiments, the antibody, or antigen binding fragment thereof, comprises a heavy chain sequence set forth in SEQ ID NO: 1013 and a light chain sequence set forth in SEQ ID NO: 1015. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 651, and a constant light chain sequence set forth in SEQ ID NO: 738. In certain embodiments, the antibody, or antigen binding fragment thereof, comprises a heavy chain sequence set forth in SEQ ID NO: 1014 and a light chain sequence set forth in SEQ ID NO: 1015.

In other aspects, described herein are methods of treating an inflammatory disorder or disease in a human patient comprising co-administering to the patient an antibody, or antigen binding fragment thereof, that binds OX40L, wherein the antibody, or antigen binding fragment thereof, comprises a CDR-H1 comprising the sequence set forth in SEQ ID NO: 44; a CDR-H2 comprising the sequence set forth in SEQ ID NO: 910; a CDR-H3 comprising the sequence set forth in SEQ ID NO: 911; a CDR-L1 comprising the sequence set forth in SEQ ID NO: 334; a CDR-L2 comprising the sequence set forth in SEQ ID NO: 361; and a CDR-L3 comprising the sequence set forth in SEQ ID NO: 912, and a hyaluronidase or variant thereof. In certain embodiments, the antibody, or antigen binding fragment thereof, that binds OX40L comprises a VH sequence set forth in SEQ ID NO: 832 and a VL sequence set forth in SEQ ID NO: 833. In certain embodiments, the antibody, or antigen binding fragment thereof comprises a heavy chain comprising a constant heavy chain sequence selected from a sequence set forth in any one of SEQ ID NOs: 637-737 and 913-1012. In certain embodiments, the antibody has a heavy chain sequence set forth in SEQ ID NO: 1018 and a light chain sequence set forth in SEQ ID NO: 1019. In certain embodiments, the antibody, or antigen binding fragment thereof comprises a heavy chain comprising a constant heavy chain sequence selected from a sequence set forth in any one of SEQ ID NOs: 637-639, 641-737, 913-915, and 917-1012. In certain embodiments, the antibody, or antigen binding fragment thereof does not comprise a constant heavy chain sequence set forth in SEQ ID NOs: 640 and 916 (i.e., a human IgG4-SPLE constant heavy chain region).

### Kits and Articles of Manufacture

The present application provides kits comprising any one or more of the antibody compositions described herein and instructions for use. In some embodiments, the kits further contain a component selected from any of secondary antibodies, reagents for immunohistochemistry analysis, a pharmaceutically acceptable excipient, a package insert, and an instruction manual and any combination thereof. In some embodiments, the kits further contain a hyaluronidase or variant thereof. In one specific embodiment, the kit comprises a pharmaceutical composition comprising any one or more of the antibody compositions described herein, with one or more pharmaceutically acceptable excipients. In some embodiments, the pharmaceutical composition further comprises a hyaluronidase or variant thereof.

The present application also provides articles of manufacture comprising any one of the antibody compositions or kits described herein. Examples of an article of manufacture include vials (including sealed vials).

In certain aspects, described herein are kits for treating an inflammatory disorder or disease in a human patient, the kit comprising: (a) a dose of any one of the antibodies, or antigen binding fragments thereof, that binds OX40L described herein (e.g., an antibody, or an antigen binding fragment thereof, that binds OX40L in unit dosage form); (b) a dose of a hyaluronidase or variant thereof (e.g., a hyaluronidase or a variant thereof in unit dosage form); and (c) instructions. In certain embodiments, the antibody, or antigen binding fragment thereof, that binds OX40L comprises a CDR-H1 comprising the sequence set forth in SEQ ID NO:1; a CDR-H2 comprising the sequence set forth in SEQ ID NO: 70; a CDR-H3 comprising the sequence set forth in SEQ ID NO: 175; a CDR-L1 comprising the sequence set forth in SEQ ID NO: 258; a CDR-L2 comprising the sequence set forth in SEQ ID NO: 335; and a CDR-L3 comprising the sequence set forth in SEQ ID NO: 368. In certain embodiments, the antibody, or antigen binding fragment thereof, that binds OX40L comprises a CDR-H1 comprising the sequence set forth in SEQ ID NO: 44; a CDR-H2 comprising the sequence set forth in SEQ ID NO: 910; a CDR-H3 comprising the sequence set forth in SEQ ID NO: 911; a CDR-L1 comprising the sequence set forth in SEQ ID NO: 334; a CDR-L2 comprising the sequence set forth in SEQ ID NO: 361; and a CDR-L3 comprising the sequence set forth in SEQ ID NO: 912. In certain embodiments, the antibody, or antigen binding fragment thereof, that binds OX40L comprises a VH sequence set forth in SEQ ID NO: 419 and a VL sequence set forth in SEQ ID NO: 489. In certain embodiments, the antibody, or antigen binding fragment thereof, that binds OX40L comprises a VH sequence set forth in SEQ ID NO: 832 and a VL sequence set forth in SEQ ID NO: 833. In certain embodiments, the antibody, or antigen binding fragment thereof, comprises a heavy chain comprising a constant heavy chain sequence set forth in SEQ ID NO: 651 or SEQ ID NO: 927. In certain embodiments, the antibody, or antigen binding fragment thereof, comprises a heavy chain comprising a constant heavy chain sequence set forth in SEQ ID NO: 651. In certain embodiments, the antibody, or antigen binding fragment thereof, comprises a heavy chain comprising a constant heavy chain sequence set forth in SEQ ID NO: 927. In certain embodiments, the antibody, or antigen binding fragment thereof, comprises a light chain comprising a constant light chain sequence set forth in SEQ ID NO: 738. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 927 and/or a constant heavy chain sequence set forth in SEQ ID NO: 651, and a constant light chain sequence set forth in SEQ ID NO: 738. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 927, and a constant light chain sequence set forth in SEQ ID NO: 738. In certain embodiments, the antibody, or antigen binding fragment thereof, comprises a heavy chain sequence set forth in SEQ ID NO: 1013 and a light chain sequence set forth in SEQ ID NO: 1015. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 651, and a constant light chain sequence set forth in SEQ ID NO: 738. In certain embodiments, the antibody, or antigen binding fragment thereof, comprises a heavy chain sequence set forth in SEQ ID NO: 1014 and a light chain sequence set forth in SEQ ID NO: 1015.

In certain embodiments, the hyaluronidase is a soluble hyaluronidase. In certain embodiments, the hyaluronidase is a recombinant human hyaluronidase or variant thereof. In certain embodiments, the hyaluronidase is a recombinant human hyaluronidase. In certain embodiments, the recombinant human hyaluronidase comprises the amino acid sequence set forth in any one of SEQ ID NOs: 843-852 and 854-909, or a mixture thereof. In certain embodiments, the recombinant human hyaluronidase is rHuPH20 (i.e., a composition comprising one or more of SEQ ID NOs: 845-850). In certain embodiments, the rHuPH20 formulation is ENHANZE®.

In certain embodiments, the kit is for use in treating an inflammatory disorder or disease. In certain embodiments, the inflammatory disorder or disease is atopic dermatitis. In certain embodiments, the inflammatory disorder or disease is asthma, idiopathic pulmonary fibrosis, alopecia areata, chronic sinusitis with nasal polyps, Chronic Rhinosinusitis without Nasal Polyps (CRSsNP), eosinophilic esophagitis (EoE), Churg-Strauss syndrome/Eosinophilic granulomatosis with polyangiitis (EGPA), Prurigo Nodularis (PN), Chronic Spontaneous Urticaria (CSU), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), lupus, rheumatoid arthritis (RA), psoriasis, an inflammatory bowel disease, such as Crohn's disease or ulcerative colitis, hidradenitis suppurativa, celiac disease, systemic sclerosis, allergic rhinitis, eosinophilic fasciitis, scleromyxedema, scleredema, or nephrogenic systemic fibrosis. In certain embodiments, the inflammatory disorder or disease is an Eosinophilic gastrointestinal disorder or disease (EGID), such as Eosinophilic Gastritis (EoG), Eosinophilic Enteritis (EoN), Eosinophilic Colitis (EoC), or Eosinophilic Gastroenteritis (EGE).

### EXAMPLE

Below is an example of a specific embodiment for carrying out the present invention. The example is offered for illustrative purposes only, and is not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed (Plenum Press) Vols A and B (1992).

### Example 1: Clinical Study

A clinical study is conducted to assess the safety, tolerability, pharmacokinetics, pharmacodynamics, and immunogenicity of an isolated antibody, or antigen binding fragment thereof, that binds OX40L administered in combination with hyaluronidase or a variant thereof. In one embodiment, the antibody, or antigen binding fragment thereof, comprises a CDR-H1 comprising the sequence set forth in SEQ ID NO: 1; a CDR-H2 comprising the sequence set forth in SEQ ID NO: 70; a CDR-H3 comprising the sequence set forth in SEQ ID NO: 175; a CDR-L1 comprising the sequence set forth in SEQ ID NO: 258; a CDR-L2 comprising the sequence set forth in SEQ ID NO: 335; and a CDR-L3 comprising the sequence set forth in SEQ ID NO: 368. In certain embodiments, the antibody, or antigen binding fragment thereof, that binds OX40L comprises a CDR-H1 comprising the sequence set forth in SEQ ID NO: 44; a CDR-H2 comprising the sequence set forth in SEQ ID NO: 910; a CDR-H3 comprising the sequence set forth in SEQ ID NO: 911; a CDR-L1 comprising the sequence set forth in SEQ ID NO: 334; a CDR-L2 comprising the sequence set forth in SEQ ID NO: 361; and a CDR-L3 comprising the sequence set forth in SEQ ID NO: 912. In certain embodiments, the antibody, or antigen binding fragment thereof, that binds OX40L comprises a VH sequence set forth in SEQ ID NO: 419 and a VL sequence set forth in SEQ ID NO: 489. In certain embodiments, the antibody, or antigen binding fragment thereof, that binds OX40L comprises a VH sequence set forth in SEQ ID NO: 832 and a VL sequence set forth in SEQ ID NO: 833. In certain embodiments, the heavy chain comprises a constant heavy chain sequence set forth in SEQ ID NO: 927 or SEQ ID NO: 651. In certain embodiments, the light chain comprises a constant light chain sequence set forth in SEQ ID NO: 738. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 927 and/or a constant heavy chain sequence set forth in SEQ ID NO: 651, and a constant light chain sequence set forth in SEQ ID NO: 738. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 927, and a constant light chain sequence set forth in SEQ ID NO: 738. In certain embodiments, the antibody, or antigen binding fragment thereof, comprises a heavy chain sequence set forth in SEQ ID NO: 1013 and a light chain sequence set forth in SEQ ID NO: 1015. In certain embodiments, the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 651, and a constant light chain sequence set forth in SEQ ID NO: 738. In certain embodiments, the antibody, or antigen binding fragment thereof, comprises a heavy chain sequence set forth in SEQ ID NO: 1014 and a light chain sequence set forth in SEQ ID NO: 1015.

In one embodiment, the hyaluronidase is a recombinant human hyaluronidase, such as rHuPH20 (i.e., a composition comprising one or more of SEQ ID NOs: 845-850). In certain embodiments, the rHuPH20 formulation is ENHANZE®.

In certain embodiments, the hyaluronidase or variant thereof and the anti-OX40L antibody, or antigen binding fragment thereof, are administered in separate formulations. In certain embodiments, the hyaluronidase or variant thereof and the anti-OX40L antibody, or antigen binding fragment thereof, are administered simultaneously in separate formulations. In certain embodiments, the hyaluronidase or variant thereof is administered to the patient prior to administration of the anti-OX40L antibody, or antigen binding fragment thereof. In certain embodiments, the hyaluronidase or variant thereof is administered to the patient after administration of the anti-OX40L antibody, or antigen binding fragment thereof. In certain embodiments, the hyaluronidase or variant thereof and the anti-OX40L antibody, or antigen binding fragment thereof, are mixed and administered in a single formulation. In certain embodiments, the hyaluronidase or variant thereof and the anti-OX40L antibody, or antigen binding fragment thereof, are administered by subcutaneous injection. In certain embodiments, the hyaluronidase or variant thereof and the anti-OX40L antibody, or antigen binding fragment thereof, are administered by intravenous injection.

In certain embodiments, the hyaluronidase is rHuPH20 administered at a concentration of 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, or 40,000 units.

Exemplary embodiments of the invention are described in the enumerated paragraphs below.
E1. A composition comprising an isolated antibody, or antigen binding fragment thereof, that binds OX40L and a hyaluronidase or variant thereof, wherein the antibody, or antigen binding fragment thereof, comprises a CDR-H1 comprising the sequence set forth in SEQ ID NO: 1; a CDR-H2 comprising the sequence set forth in SEQ ID NO: 70; a CDR-H3 comprising the sequence set forth in SEQ ID NO: 175; a CDR-L1 comprising the sequence set forth in SEQ ID NO: 258; a CDR-L2 comprising the sequence set forth in SEQ ID NO: 335; and a CDR-L3 comprising the sequence set forth in SEQ ID NO: 368.
E2. The composition of E1, wherein the antibody, or antigen binding fragment thereof, comprises a VH sequence set forth in SEQ ID NO: 419 and a VL sequence set forth in SEQ ID NO: 489.
E3. A composition comprising an isolated antibody, or antigen binding fragment thereof, that binds OX40L and a hyaluronidase or variant thereof, wherein the antibody, or antigen binding fragment thereof, comprises a CDR-H1 comprising the sequence set forth in SEQ ID NO: 44; a CDR-H2 comprising the sequence set forth in SEQ ID NO: 910; a CDR-H3 comprising the sequence set forth in SEQ ID NO: 911; a CDR-L1 comprising the sequence set forth in SEQ ID NO: 334; a CDR-L2 comprising the sequence set forth in SEQ ID NO: 361; and a CDR-L3 comprising the sequence set forth in SEQ ID NO: 912.
E4. The composition of E3, wherein the antibody, or antigen binding fragment thereof, comprises a VH sequence set forth in SEQ ID NO: 832 and a VL sequence set forth in SEQ ID NO: 833.
E5. The composition of any one of E1-E4, wherein the antibody, or antigen binding fragment thereof, is a humanized, human, or chimeric antibody.
E6. The composition of E5, wherein the antibody, or antigen binding fragment thereof, is a humanized antibody.
E7. The composition of any one of E1-E6, wherein the antibody, or antigen binding fragment thereof, comprises a heavy chain human constant region of a class selected from IgG, IgA, IgD, IgE, and IgM.
E8. The composition of E7, wherein the antibody, or antigen binding fragment thereof, comprises a heavy chain human constant region of the class IgG and a subclass selected from IgG1, IgG2, IgG3, and IgG4.
E9. The composition of any one of E1-E8, wherein the antibody, or antigen binding fragment thereof, comprises a human IgG1 Fc region.
E10. The composition of any one of E1-E9, wherein the antibody, or antigen binding fragment thereof, comprises a human Fc region comprising a human IgG sequence selected from a sequence set forth in any one of SEQ ID NOs: 637-737 and 913-1012.
E11. The composition of any one of E1-E10, wherein the antibody, or antigen binding fragment thereof, comprises a heavy chain comprising a constant heavy chain region having a means for extending the half-life of the antibody.
E12. The composition of any one of E1-E11, wherein the antibody, or antigen binding fragment thereof, comprises a light chain comprising a constant light chain sequence set forth in SEQ ID NO: 738.
E13. The composition of any one of E1-E12, wherein the antibody, or antigen binding fragment thereof, comprises an Fc region comprising one or more amino acid substitutions, wherein the one or more amino acid substitutions result in an increase in one or more of antibody half-life, ADCC activity, ADCP activity, or CDC activity compared with the Fc region without the one or more amino acid substitutions.
E14. The composition of any one of E1-E12, wherein the antibody, or antigen binding fragment thereof, comprises an Fc region comprising one or more amino acid substitutions, wherein the one or more amino acid substitutions result in a decrease in one or more of, ADCC activity, ADCP activity or CDC activity compared to an antibody comprising a wild-type Fc region.
E15. The composition of E13 or E14, wherein the one or more amino acid substitutions is selected from the group consisting of S228P, M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W; optionally, wherein the one or more amino acid substitutions comprises a plurality of amino acid substitutions selected from the group consisting of M428L/N434S (LS), M252Y/S254T/T256E (YTE), T250Q/M428L, T307A/E380A/N434A, T256D/T307Q (DQ), T256D/T307W (DW), M252Y/T256D (YD), T307Q/Q311V/A378V (QVV), T256D/H285D/T307R/Q311V/A378V (DDRVV), L309D/Q311H/N434S (DHS), S228P/L235E (SPLE), L234A/L235A (LALA), M428L/N434A (LA), L235A/G237A (LAGA), L234A/L235A/G237A (LALAGA), L234A/L235A/P329G (LALAPG), D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/N434A, D265A/N434A, LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV.
E16. The composition of E15, wherein the one or more amino acid substitutions is selected from the group consisting of LS, YTE, T250Q/M428L, T307A/E380A/N434A, DQ, DW, YD, QVV, DHS, LA, D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, N297A/QVV, D265A/QVV, LALA/QVV, LAGA/QVV, LALAGA/QVV, and LALAPG/QVV.
E17. The composition of any one of E1-E16, wherein the antibody, or antigen binding fragment thereof, comprises a human IgG1 Fc region with LALA and YTE mutations.
E18. The composition of any one of E1-E17, wherein the antibody, or antigen binding fragment thereof, comprises a human IgG1 Fc region with LALA mutations at positions L235A/L236A by direct numbering and YTE mutations at positions M253Y/S255T/T257E by direct numbering.
E19. The composition of any one of E1-E18, wherein the antibody, or antigen binding fragment thereof, comprises a heavy chain comprising a constant heavy chain sequence set forth in SEQ ID NO: 651 and/or SEQ ID NO: 927.
E20. The composition of E19, wherein the antibody, or antigen binding fragment thereof, comprises a heavy chain comprising a constant heavy chain sequence set forth in SEQ ID NO: 927.
E21. The composition of E19, wherein the antibody, or antigen binding fragment thereof, comprises a heavy chain comprising a constant heavy chain sequence set forth in SEQ ID NO: 651.
E22. The composition of any one of E1, E2, and E5-E21, wherein the antibody, or antigen binding fragment thereof, comprises a VH domain comprising the amino acid sequence set forth in SEQ ID NO: 419, a VL domain comprising the amino acid sequence set forth in SEQ ID NO: 489, and a human IgG1 Fc region comprising LALA and YTE substitutions.
E23. The composition of any one of E1, E2, and E5-E22, wherein the antibody, or antigen binding fragment thereof, comprises a VH domain comprising the amino acid sequence set forth in SEQ ID NO: 419, a VL domain comprising the amino acid sequence set forth in SEQ ID NO: 489, a constant heavy chain sequence set forth in SEQ ID NO: 927 and/or a constant heavy chain sequence set forth in SEQ ID NO: 651, and a constant light chain sequence set forth in SEQ ID NO: 738.
E24. The composition of E23, wherein the antibody, or antigen binding fragment thereof, comprises a VH domain comprising the amino acid sequence set forth in SEQ ID NO: 419, a VL domain comprising the amino acid sequence set forth in SEQ ID NO: 489, a constant heavy chain sequence set forth in SEQ ID NO: 927, and a constant light chain sequence set forth in SEQ ID NO: 738.
E25. The composition of E24, wherein the antibody, or antigen binding fragment thereof, comprises a heavy chain sequence set forth in SEQ ID NO: 1013 and a light chain sequence set forth in SEQ ID NO: 1015.
E26. The composition of E23, wherein the antibody, or antigen binding fragment thereof, comprises a VH domain comprising the amino acid sequence set forth in SEQ ID NO: 419, a VL domain comprising the amino acid sequence set forth in SEQ ID NO: 489, a constant heavy chain sequence set forth in SEQ ID NO: 651, and a constant light chain sequence set forth in SEQ ID NO: 738.
E27. The composition of E26, wherein the antibody, or antigen binding fragment thereof, comprises a heavy chain sequence set forth in SEQ ID NO: 1014 and a light chain sequence set forth in SEQ ID NO: 1015.
E28. The composition of any one of E1-E27, wherein the Fc region of the antibody, or antigen binding fragment thereof, binds to Neonatal Fc receptor (FcRn).
E29. The composition of E28, wherein the Fc region of the antibody, or antigen binding fragment thereof, binds an FcRn with higher affinity at pH 6.0 compared to an antibody comprising a wild-type Fc region.
E30. The composition of E28 or E29, wherein the Fc region of the antibody, or antigen binding fragment thereof, binds to FcRn with a K_{D} of <1×10⁻⁷ M at pH 6.0.
E31. The composition of any one of E1-E30, wherein the antibody, or antigen binding fragment thereof, is a monoclonal antibody.
E32. The composition of any one of E1-E31, wherein the antibody, or antigen binding fragment thereof, binds an OX40L sequence set forth in SEQ ID NO: 739.
E33. The composition of any one of E1-E32, wherein the hyaluronidase is a recombinant human hyaluronidase.
E34. The composition of E33, wherein the recombinant human hyaluronidase comprises an amino acid sequence set forth in any one of SEQ ID NOs: 843-852 and 854-909 (e.g., SEQ ID NO: 845), or a mixture thereof.
E35. The composition of E33, wherein the recombinant human hyaluronidase is a soluble hyaluronidase provided as a composition designated rHuPH20.
E36. The composition of E35, wherein the rHuPH20 formulation is ENHANZE®. E37. The composition of any one of E1-E36, wherein the composition is formulated for administration by subcutaneous injection.
E38. The composition of any one of E1-E36, wherein the composition is formulated for administration by intravenous injection.
E39. A combination comprising an isolated antibody, or antigen binding fragment thereof, that binds OX40L and a hyaluronidase or variant thereof, wherein the antibody, or antigen binding fragment thereof, comprises a CDR-H1 comprising the sequence set forth in SEQ ID NO: 1; a CDR-H2 comprising the sequence set forth in SEQ ID NO: 70; a CDR-H3 comprising the sequence set forth in SEQ ID NO: 175; a CDR-L1 comprising the sequence set forth in SEQ ID NO: 258; a CDR-L2 comprising the sequence set forth in SEQ ID NO: 335; and a CDR-L3 comprising the sequence set forth in SEQ ID NO: 368.
E40. The combination of E39, wherein the antibody, or antigen binding fragment thereof, comprises a VH sequence set forth in SEQ ID NO: 419 and a VL sequence set forth in SEQ ID NO: 489.
E41. A combination comprising an isolated antibody, or antigen binding fragment thereof, that binds OX40L and a hyaluronidase or variant thereof, wherein the antibody, or antigen binding fragment thereof, comprises a CDR-H1 comprising the sequence set forth in SEQ ID NO: 44; a CDR-H2 comprising the sequence set forth in SEQ ID NO: 910; a CDR-H3 comprising the sequence set forth in SEQ ID NO: 911; a CDR-L1 comprising the sequence set forth in SEQ ID NO: 334; a CDR-L2 comprising the sequence set forth in SEQ ID NO: 361; and a CDR-L3 comprising the sequence set forth in SEQ ID NO: 912.
E42. The combination of E41, wherein the antibody, or antigen binding fragment thereof, comprises a VH sequence set forth in SEQ ID NO: 832 and a VL sequence set forth in SEQ ID NO: 833.
E43. The combination of any one of E39-E42, wherein the antibody, or antigen binding fragment thereof, is a humanized, human, or chimeric antibody.
E44. The combination of E43, wherein the antibody, or antigen binding fragment thereof, is a humanized antibody.
E45. The combination of any one of E39-E44, wherein the antibody, or antigen binding fragment thereof, comprises a heavy chain human constant region of a class selected from IgG, IgA, IgD, IgE, and IgM.
E46. The combination of E45, wherein the antibody, or antigen binding fragment thereof, comprises a heavy chain human constant region of the class IgG and a subclass selected from IgG1, IgG2, IgG3, and IgG4.
E47. The combination of any one of E39-E46, wherein the antibody, or antigen binding fragment thereof, comprises a human IgG1 Fc region.
E48. The combination of any one of E39-E47, wherein the antibody, or antigen binding fragment thereof, comprises a human Fc region comprising a human IgG sequence selected from a sequence set forth in any one of SEQ ID NOs: 637-737 and 913-1012.
E49. The combination of any one of E39-E48, wherein the antibody, or antigen binding fragment thereof, comprises a heavy chain comprising a constant heavy chain region having a means for extending the half-life of the antibody.
E50. The combination of any one of E39-E49, wherein the antibody, or antigen binding fragment thereof, comprises a light chain comprising a constant light chain sequence set forth in SEQ ID NO: 738.
E51. The combination of any one of E39-E50, wherein the antibody, or antigen binding fragment thereof, comprises an Fc region comprising one or more amino acid substitutions, wherein the one or more amino acid substitutions result in an increase in one or more of antibody half-life, ADCC activity, ADCP activity, or CDC activity compared with the Fc region without the one or more amino acid substitutions.
E52. The combination of any one of E39-E50, wherein the antibody, or antigen binding fragment thereof, comprises an Fc region comprising one or more amino acid substitutions, wherein the one or more amino acid substitutions result in a decrease in one or more of, ADCC activity, ADCP activity or CDC activity compared to an antibody comprising a wild-type Fc region.
E53. The combination of E51 or E52, wherein the one or more amino acid substitutions is selected from the group consisting of S228P, M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W; optionally, wherein the one or more amino acid substitutions comprises a plurality of amino acid substitutions selected from the group consisting of M428L/N434S (LS), M252Y/S254T/T256E (YTE), T250Q/M428L, T307A/E380A/N434A, T256D/T307Q (DQ), T256D/T307W (DW), M252Y/T256D (YD), T307Q/Q311V/A378V (QVV), T256D/H285D/T307R/Q311V/A378V (DDRVV), L309D/Q311H/N434S (DHS), S228P/L235E (SPLE), L234A/L235A (LALA), M428L/N434A (LA), L235A/G237A (LAGA), L234A/L235A/G237A (LALAGA), L234A/L235A/P329G (LALAPG), D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/N434A, D265A/N434A, LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV.
E54. The combination of E53, wherein the one or more amino acid substitutions is selected from the group consisting of LS, YTE, T250Q/M428L, T307A/E380A/N434A, DQ, DW, YD, QVV, DHS, LA, D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, N297A/QVV, D265A/QVV, LALA/QVV, LAGA/QVV, LALAGA/QVV, and LALAPG/QVV.
E55. The combination of any one of E39-E54, wherein the antibody, or antigen binding fragment thereof, comprises a human IgG1 Fc region with LALA and YTE mutations.
E56. The combination of any one of E39-E55, wherein the antibody, or antigen binding fragment thereof, comprises a human IgG1 Fc region with LALA mutations at positions L235A/L236A by direct numbering and YTE mutations at positions M253Y/S255T/T257E by direct numbering.
E57. The combination of any one of E39-E56, wherein the antibody, or antigen binding fragment thereof, comprises a heavy chain comprising a constant heavy chain sequence set forth in SEQ ID NO: 651 and/or SEQ ID NO: 927.
E58. The combination of E57, wherein the antibody, or antigen binding fragment thereof, comprises a heavy chain comprising a constant heavy chain sequence set forth in SEQ ID NO: 927.
E59. The combination of E57, wherein the antibody, or antigen binding fragment thereof, comprises a heavy chain comprising a constant heavy chain sequence set forth in SEQ ID NO: 651.
E60. The combination of any one of E39, E40, and E43-E59, wherein the antibody, or antigen binding fragment thereof, comprises a VH domain comprising the amino acid sequence set forth in SEQ ID NO: 419, a VL domain comprising the amino acid sequence set forth in SEQ ID NO: 489, and a human IgG1 Fc region comprising LALA and YTE substitutions.
E61. The combination of any one of E39, E40, and E43-E60, wherein the antibody, or antigen binding fragment thereof, comprises a VH domain comprising the amino acid sequence set forth in SEQ ID NO: 419, a VL domain comprising the amino acid sequence set forth in SEQ ID NO: 489, a constant heavy chain sequence set forth in SEQ ID NO: 927 and/or a constant heavy chain sequence set forth in SEQ ID NO: 651, and a constant light chain sequence set forth in SEQ ID NO: 738.
E62. The combination of E61, wherein the antibody, or antigen binding fragment thereof, comprises a VH domain comprising the amino acid sequence set forth in SEQ ID NO: 419, a VL domain comprising the amino acid sequence set forth in SEQ ID NO: 489, a constant heavy chain sequence set forth in SEQ ID NO: 927, and a constant light chain sequence set forth in SEQ ID NO: 738.
E63. The combination of E62, wherein the antibody, or antigen binding fragment thereof, comprises a heavy chain sequence set forth in SEQ ID NO: 1013 and a light chain sequence set forth in SEQ ID NO: 1015.
E64. The combination of E61, wherein the antibody, or antigen binding fragment thereof, comprises a VH domain comprising the amino acid sequence set forth in SEQ ID NO: 419, a VL domain comprising the amino acid sequence set forth in SEQ ID NO: 489, a constant heavy chain sequence set forth in SEQ ID NO: 651, and a constant light chain sequence set forth in SEQ ID NO: 738.
E65. The combination of E64, wherein the antibody, or antigen binding fragment thereof, comprises a heavy chain sequence set forth in SEQ ID NO: 1014 and a light chain sequence set forth in SEQ ID NO: 1015.
E66. The combination of any one of E39-E65, wherein the Fc region of the antibody, or antigen binding fragment thereof, binds to Neonatal Fc receptor (FcRn).
E67. The combination of E66, wherein the Fc region of the antibody, or antigen binding fragment thereof, binds an FcRn with higher affinity at pH 6.0 compared to an antibody comprising a wild-type Fc region.
E68. The combination of E66 or E67, wherein the Fc region of the antibody, or antigen binding fragment thereof, binds to FcRn with a K_{D} of <1×10⁻⁷ M at pH 6.0.
E69. The combination of any one of E39-E68, wherein the antibody, or antigen binding fragment thereof, is a monoclonal antibody.
E70. The combination of any one of E39-E69, wherein the antibody, or antigen binding fragment thereof, binds an OX40L sequence set forth in SEQ ID NO: 739.
E71. The combination of any one of E39-E70, wherein the hyaluronidase is a recombinant human hyaluronidase.
E72. The combination of E71, wherein the recombinant human hyaluronidase comprises an amino acid sequence set forth in any one of SEQ ID NOs: 843-852 and 854-909 (e.g., SEQ ID NO: 845), or a mixture thereof.
E73. The combination of E71, wherein the recombinant human hyaluronidase is a soluble hyaluronidase provided as a composition designated rHuPH20.
E74. The combination of E73, wherein the rHuPH20 formulation is ENHANZE®.
E75. The combination of any one of E39-E74, wherein the hyaluronidase and the antibody, or antigen binding fragment thereof, are administered in separate formulations.
E76. The combination of E75, wherein the hyaluronidase and the antibody, or antigen binding fragment thereof, are administered simultaneously in separate formulations.
E77. The combination of E75, wherein the hyaluronidase is administered to the patient prior to administration of the antibody, or antigen binding fragment thereof.
E78. The combination of E75, wherein the hyaluronidase is administered to the patient after administration of the antibody, or antigen binding fragment thereof.
E79. The combination of any one of E39-E74, wherein the hyaluronidase and the antibody, or antigen binding fragment thereof, are administered in a single formulation.
E80. The combination of any one of E39-E79, wherein the hyaluronidase and the antibody, or antigen binding fragment thereof, are administered by subcutaneous injection.
E81. The combination of any one of E39-E79, wherein the hyaluronidase and the antibody, or antigen binding fragment thereof, are administered by intravenous injection.
E82. The combination of any one of E39-E81, wherein the hyaluronidase is rHuPH20 administered at a concentration of 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, or 40,000 units.
E83. The composition of any one of E1-E38 or the combination of any one of E39-E82, for use in the treatment of an inflammatory disorder or a disease.
E84. The composition or combination for use according to E83, wherein the inflammatory disorder or disease is atopic dermatitis.
E85. The composition or combination for use according to E83, wherein the inflammatory disorder or disease is asthma, chronic sinusitis with nasal polyps, Chronic Rhinosinusitis without Nasal Polyps (CRSsNP), eosinophilic esophagitis (EoE), an EGID, such as Eosinophilic Gastritis (EoG), Eosinophilic Enteritis (EoN), Eosinophilic Colitis (EoC), or Eosinophilic Gastroenteritis (EGE), Churg-Strauss syndrome/Eosinophilic granulomatosis with polyangiitis (EGPA), Prurigo Nodularis (PN), Chronic Spontaneous Urticaria (CSU), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), lupus, rheumatoid arthritis (RA), psoriasis, an inflammatory bowel disease, such as ulcerative colitis or Crohn's disease, hidradenitis suppurativa, celiac disease, idiopathic pulmonary fibrosis, alopecia areata, allergic rhinitis, eosinophilic fasciitis, scleromyxedema, scleredema, nephrogenic systemic fibrosis, or systemic sclerosis.
E86. A method of treating an inflammatory disorder or disease in a human patient in need thereof, comprising administering to the patient an isolated antibody, or antigen binding fragment thereof, that binds OX40L and a hyaluronidase or variant thereof, wherein the antibody, or antigen binding fragment thereof, comprises a CDR-H1 comprising the sequence set forth in SEQ ID NO: 1; a CDR-H2 comprising the sequence set forth in SEQ ID NO: 70; a CDR-H3 comprising the sequence set forth in SEQ ID NO: 175; a CDR-L1 comprising the sequence set forth in SEQ ID NO: 258; a CDR-L2 comprising the sequence set forth in SEQ ID NO: 335; and a CDR-L3 comprising the sequence set forth in SEQ ID NO: 368.
E87. The method of E86, wherein the antibody, or antigen binding fragment thereof, comprises a VH sequence set forth in SEQ ID NO: 419 and a VL sequence set forth in SEQ ID NO: 489.
E88. A method of treating an inflammatory disorder or disease in a human patient in need thereof, comprising administering to the patient an isolated antibody, or antigen binding fragment thereof, that binds OX40L and a hyaluronidase or variant thereof, wherein the antibody, or antigen binding fragment thereof, comprises a CDR-H1 comprising the sequence set forth in SEQ ID NO: 44; a CDR-H2 comprising the sequence set forth in SEQ ID NO: 910; a CDR-H3 comprising the sequence set forth in SEQ ID NO: 911; a CDR-L1 comprising the sequence set forth in SEQ ID NO: 334; a CDR-L2 comprising the sequence set forth in SEQ ID NO: 361; and a CDR-L3 comprising the sequence set forth in SEQ ID NO: 912.
E89. The method of E88, wherein the antibody, or antigen binding fragment thereof, comprises a VH sequence set forth in SEQ ID NO: 832 and a VL sequence set forth in SEQ ID NO: 833.
E90. The method of any one of E86-E89, wherein the antibody, or antigen binding fragment thereof, is a humanized, human, or chimeric antibody.
E91. The method of E90, wherein the antibody, or antigen binding fragment thereof, is a humanized antibody.
E92. The method of any one of E86-E91, wherein the antibody, or antigen binding fragment thereof, comprises a heavy chain human constant region of a class selected from IgG, IgA, IgD, IgE, and IgM.
E93. The method of E92, wherein the antibody, or antigen binding fragment thereof, comprises a heavy chain human constant region of the class IgG and a subclass selected from IgG1, IgG2, IgG3, and IgG4.
E94. The method of any one of E86-E93, wherein the antibody, or antigen binding fragment thereof, comprises a human IgG1 Fc region.
E95. The method of any one of E86-E94, wherein the antibody, or antigen binding fragment thereof, comprises a human Fc region comprising a human IgG sequence selected from a sequence set forth in any one of SEQ ID NOs: 637-737 and 913-1012.
E96. The method of any one of E86-E95, wherein the antibody, or antigen binding fragment thereof, comprises a heavy chain comprising a constant heavy chain region having a means for extending the half-life of the antibody.
E97. The method of any one of E86-E96, wherein the antibody, or antigen binding fragment thereof, comprises a light chain comprising a constant light chain sequence set forth in SEQ ID NO: 738.
E98. The method of any one of E86-E97, wherein the antibody, or antigen binding fragment thereof, comprises an Fc region comprising one or more amino acid substitutions, wherein the one or more amino acid substitutions result in an increase in one or more of antibody half-life, ADCC activity, ADCP activity, or CDC activity compared with the Fc region without the one or more amino acid substitutions.
E99. The method of any one of E86-E97, wherein the antibody, or antigen binding fragment thereof, comprises an Fc region comprising one or more amino acid substitutions, wherein the one or more amino acid substitutions result in a decrease in one or more of ADCC activity, ADCP activity or CDC activity compared to an antibody comprising a wild-type Fc region.
E100. The method of E98 or E99, wherein the one or more amino acid substitutions is selected from the group consisting of S228P, M252Y, S254T, T256E, T256D, T250Q, H285D, T307A, T307Q, T307R, T307W, L309D, Q411H, Q311V, A378V, E380A, M428L, N434A, N434S, N297A, D265A, L234A, L235A, and N434W; optionally, wherein the one or more amino acid substitutions comprises a plurality of amino acid substitutions selected from the group consisting of M428L/N434S (LS), M252Y/S254T/T256E (YTE), T250Q/M428L, T307A/E380A/N434A, T256D/T307Q (DQ), T256D/T307W (DW), M252Y/T256D (YD), T307Q/Q311V/A378V (QVV), T256D/H285D/T307R/Q311V/A378V (DDRVV), L309D/Q311H/N434S (DHS), S228P/L235E (SPLE), L234A/L235A (LALA), M428L/N434A (LA), L235A/G237A (LAGA), L234A/L235A/G237A (LALAGA), L234A/L235A/P329G (LALAPG), D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/N434A, D265A/N434A, LALA/N434A, LAGA/N434A, LALAGA/N434A, LALAPG/N434A, N297A/N434W, D265A/N434W, LALA/N434W, LAGA/N434W, LALAGA/N434W, LALAPG/N434W, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, T307Q/Q311V/A378V (QVV), N297A/QVV, D265A/QVV, LALA/QVV, LAGA/QVV, LALAGA/QVV, LALAPG/QVV, DDRVV, N297A/DDRVV, D265A/DDRVV, LALA/DDRVV, LAGA/DDRVV, LALAGA/DDRVV, and LALAPG/DDRVV.
E101. The method of E100, wherein the one or more amino acid substitutions is selected from the group consisting of LS, YTE, T250Q/M428L, T307A/E380A/N434A, DQ, DW, YD, QVV, DHS, LA, D265A/YTE, LALA/YTE, LAGA/YTE, LALAGA/YTE, LALAPG/YTE, N297A/LS, D265A/LS, LALA/LS, LALAGA/LS, LALAPG/LS, N297A/DHS, D265A/DHS, LALA/DHS, LAGA/DHS, LALAGA/DHS, LALAPG/DHS, SP/YTE, SPLE/YTE, SP/LS, SPLE/LS, SP/DHS, SPLE/DHS, N297A/LA, D265A/LA, LALA/LA, LAGA/LA, LALAGA/LA, LALAPG/LA, N297A/DQ, D265A/DQ, LALA/DQ, LAGA/DQ, LALAGA/DQ, LALAPG/DQ, N297A/DW, D265A/DW, LALA/DW, LAGA/DW, LALAGA/DW, LALAPG/DW, N297A/YD, D265A/YD, LALA/YD, LAGA/YD, LALAGA/YD, LALAPG/YD, N297A/QVV, D265A/QVV, LALA/QVV, LAGA/QVV, LALAGA/QVV, and LALAPG/QVV.
E102. The method of any one of E86-E101, wherein the antibody, or antigen binding fragment thereof, comprises a human IgG1 Fc region with LALA and YTE mutations.
E103. The method of any one of E86-E102, wherein the antibody, or antigen binding fragment thereof, comprises a human IgG1 Fc region with LALA mutations at positions L235A/L236A by direct numbering and YTE mutations at positions M253Y/S255T/T257E by direct numbering.
E104. The method of any one of E86-E103, wherein the antibody, or antigen binding fragment thereof, comprises a heavy chain comprising a constant heavy chain sequence set forth in SEQ ID NO: 651 and/or SEQ ID NO: 927.
E105. The method of E104, wherein the antibody, or antigen binding fragment thereof, comprises a heavy chain comprising a constant heavy chain sequence set forth in SEQ ID NO: 927.
E106. The method of E104, wherein the antibody, or antigen binding fragment thereof, comprises a heavy chain comprising a constant heavy chain sequence set forth in SEQ ID NO: 651.
E107. The method of any one of E86, E87, and E90-E106, wherein the antibody, or antigen binding fragment thereof, comprises a VH domain comprising the amino acid sequence set forth in SEQ ID NO: 419, a VL domain comprising the amino acid sequence set forth in SEQ ID NO: 489, and a human IgG1 Fc region comprising LALA and YTE substitutions.
E108. The method of any one of E86, E87, and E90-E107, wherein the antibody, or antigen binding fragment thereof, comprises a VH domain comprising the amino acid sequence set forth in SEQ ID NO: 419, a VL domain comprising the amino acid sequence set forth in SEQ ID NO: 489, a constant heavy chain sequence set forth in SEQ ID NO: 927 and/or a constant heavy chain sequence set forth in SEQ ID NO: 651, and a constant light chain sequence set forth in SEQ ID NO: 738.
E109. The method of E108, wherein the antibody, or antigen binding fragment thereof, comprises a VH domain comprising the amino acid sequence set forth in SEQ ID NO: 419, a VL domain comprising the amino acid sequence set forth in SEQ ID NO: 489, a constant heavy chain sequence set forth in SEQ ID NO: 927, and a constant light chain sequence set forth in SEQ ID NO: 738.
E110. The method of E109, wherein the antibody, or antigen binding fragment thereof, comprises a heavy chain sequence set forth in SEQ ID NO: 1013 and a light chain sequence set forth in SEQ ID NO: 1015.
E111. The method of E108, wherein the antibody, or antigen binding fragment thereof, comprises a VH domain comprising the amino acid sequence set forth in SEQ ID NO: 419, a VL domain comprising the amino acid sequence set forth in SEQ ID NO: 489, a constant heavy chain sequence set forth in SEQ ID NO: 651, and a constant light chain sequence set forth in SEQ ID NO: 738.
E112. The method of E111, wherein the antibody, or antigen binding fragment thereof, comprises a heavy chain sequence set forth in SEQ ID NO: 1014 and a light chain sequence set forth in SEQ ID NO: 1015.
E113. The method of any one of E86-E112, wherein the Fc region of the antibody, or antigen binding fragment thereof, binds to Neonatal Fc receptor (FcRn).
E114. The method of E113, wherein the Fc region of the antibody, or antigen binding fragment thereof, binds an FcRn with higher affinity at pH 6.0 compared to an antibody comprising a wild-type Fc region.
E115. The method of E113 or E114, wherein the Fc region of the antibody, or antigen binding fragment thereof, binds to FcRn with a K_{D} of <1×10⁻⁷ M at pH 6.0.
E116. The method of any one of E86-E115, wherein the antibody, or antigen binding fragment thereof, is a monoclonal antibody.
E117. The method of any one of E86-E116, wherein the antibody, or antigen binding fragment thereof, binds an OX40L sequence set forth in SEQ ID NO: 739.
E118. The method of any one of E86-E117, wherein the hyaluronidase is a recombinant human hyaluronidase.
E119. The method of E118, wherein the recombinant human hyaluronidase comprises an amino acid sequence set forth in any one of SEQ ID NOs: 843-852 and 854-909 (e.g., SEQ ID NO: 845), or a mixture thereof.
E120. The method of E118, wherein the recombinant human hyaluronidase is a soluble hyaluronidase provided as a composition designated rHuPH20.
E121. The method of E120, wherein the rHuPH20 formulation is ENHANZE®.
E122. The method of any one of E86-E121, wherein the hyaluronidase and the antibody, or antigen binding fragment thereof, are administered in separate formulations.
E123. The method of E122, wherein the hyaluronidase and the antibody, or antigen binding fragment thereof, are administered simultaneously in separate formulations.
E124. The method of E122, wherein the hyaluronidase is administered to the patient prior to administration of the antibody, or antigen binding fragment thereof.
E125. The method of E122, wherein the hyaluronidase is administered to the patient after administration of the antibody, or antigen binding fragment thereof.
E126. The method of any one of E86-E121, wherein the hyaluronidase and the antibody, or antigen binding fragment thereof, are administered in a single formulation.
E127. The method of any one of E86-E126, wherein the hyaluronidase and the antibody, or antigen binding fragment thereof, are administered by subcutaneous injection.
E128. The method of any one of E86-E126, wherein the hyaluronidase and the antibody, or antigen binding fragment thereof, are administered by intravenous injection.
E129. The method of any one of E86-E128, wherein the hyaluronidase is rHuPH20 administered at a concentration of 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, or 40,000 units.
E130. The method of any one of E86-E129, wherein the inflammatory disorder or disease is atopic dermatitis.
E131. The method of any one of E86-E129, wherein the inflammatory disorder or disease is asthma, chronic sinusitis with nasal polyps, Chronic Rhinosinusitis without Nasal Polyps (CRSsNP), eosinophilic esophagitis (EoE), an EGID, such as Eosinophilic Gastritis (EoG), Eosinophilic Enteritis (EoN), Eosinophilic Colitis (EoC), or Eosinophilic Gastroenteritis (EGE), Churg-Strauss syndrome/Eosinophilic granulomatosis with polyangiitis (EGPA), Prurigo Nodularis (PN), Chronic Spontaneous Urticaria (CSU), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), lupus, rheumatoid arthritis (RA), psoriasis, an inflammatory bowel disease, such as ulcerative colitis or Crohn's disease, hidradenitis suppurativa, celiac disease, idiopathic pulmonary fibrosis, alopecia areata, allergic rhinitis, eosinophilic fasciitis, scleromyxedema, scleredema, nephrogenic systemic fibrosis, or systemic sclerosis.
E132. A kit comprising:
   (a) an antibody, or antigen binding fragment thereof, that binds OX40L in unit dosage form, wherein the antibody comprises a VH sequence set forth in SEQ ID NO: 419 and a VL sequence set forth in SEQ ID NO: 489;
   (b) a hyaluronidase or variant thereof in unit dosage form; and
   (c) instructions.
E133. The kit of E132, wherein the hyaluronidase is a recombinant human hyaluronidase.
E134. The kit of E133, wherein the recombinant human hyaluronidase comprises an amino acid sequence set forth in any one of SEQ ID NOs: 843-852 and 854-909 (e.g., SEQ ID NO: 845), or a mixture thereof.
E135. The kit of E133, wherein the recombinant human hyaluronidase is a soluble hyaluronidase provided as a composition designated rHuPH20.
E136. The kit of E135, wherein the rHuPH20 formulation is ENHANZE®.
E137. The kit of any one of E132-E136, for use in treating an inflammatory disorder or disease.
E138. The kit of E137, wherein the inflammatory disorder or disease is atopic dermatitis.
E139. The kit of E137, wherein the inflammatory disorder or disease is asthma, chronic sinusitis with nasal polyps, Chronic Rhinosinusitis without Nasal Polyps (CRSsNP), eosinophilic esophagitis (EoE), an EGID, such as Eosinophilic Gastritis (EoG), Eosinophilic Enteritis (EoN), Eosinophilic Colitis (EoC), or Eosinophilic Gastroenteritis (EGE), Churg-Strauss syndrome/Eosinophilic granulomatosis with polyangiitis (EGPA), Prurigo Nodularis (PN), Chronic Spontaneous Urticaria (CSU), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), lupus, rheumatoid arthritis (RA), psoriasis, an inflammatory bowel disease, such as ulcerative colitis or Crohn's disease, hidradenitis suppurativa, celiac disease, idiopathic pulmonary fibrosis, alopecia areata, allergic rhinitis, eosinophilic fasciitis, scleromyxedema, scleredema, nephrogenic systemic fibrosis, or systemic sclerosis.
E140. The kit of any one of E132-E139, wherein the antibody, or antigen binding fragment thereof, comprises a human Fc region comprising a human IgG sequence selected from a sequence set forth in any one of SEQ ID NOs: 637-737 and 913-1012.
E141. The kit of any one of E132-E140, wherein the antibody, or antigen binding fragment thereof, comprises a light chain comprising a constant light chain sequence set forth in SEQ ID NO: 738.
E142. The kit of any one of E132-E141, wherein the antibody, or antigen binding fragment thereof, comprises a human IgG1 Fc region with LALA and YTE mutations.
E143. The kit of any one of E132-E142, wherein the antibody, or antigen binding fragment thereof, comprises a human IgG1 Fc with LALA mutations at positions L235A/L236A by direct numbering and YTE mutations at positions M253Y/S255T/T257E by direct numbering.
E144. The kit of any one of E143-E143, wherein the antibody, or antigen binding fragment thereof, comprises a heavy chain comprising a constant heavy chain sequence set forth in SEQ ID NO: 651 and/or SEQ ID NO: 927.
E145. The kit of E144, wherein the antibody, or antigen binding fragment thereof, comprises a VH domain comprising the amino acid sequence set forth in SEQ ID NO: 419, a VL domain comprising the amino acid sequence set forth in SEQ ID NO: 489, a constant heavy chain sequence set forth in SEQ ID NO: 927, and a constant light chain sequence set forth in SEQ ID NO: 738.
E146. The kit of E145, wherein the antibody, or antigen binding fragment thereof, comprises a heavy chain sequence set forth in SEQ ID NO: 1013 and a light chain sequence set forth in SEQ ID NO: 1015.
E147. The kit of E144, wherein the antibody, or antigen binding fragment thereof, comprises a VH domain comprising the amino acid sequence set forth in SEQ ID NO: 419, a VL domain comprising the amino acid sequence set forth in SEQ ID NO: 489, a constant heavy chain sequence set forth in SEQ ID NO: 651, and a constant light chain sequence set forth in SEQ ID NO: 738.
E148. The kit of E147, wherein the antibody, or antigen binding fragment thereof, comprises a heavy chain sequence set forth in SEQ ID NO: 1014 and a light chain sequence set forth in SEQ ID NO: 1015.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

Informal Sequence Listing

| | | SEQ ID |
|---|---|---|
| Description | Sequence | NO |
| Kabat HCDR1 | NYAMT | SEQ ID |
| | | NO: 1 |
| Kabat HCDR1 | SYAMN | SEQ ID |
| | | NO: 2 |
| Kabat HCDR1 | SNYMS | SEQ ID |
| | | NO: 3 |
| Kabat HCDR1 | NYWMH | SEQ ID |
| | | NO: 4 |
| Kabat HCDR1 | NAWMS | SEQ ID |
| | | NO: 5 |
| Kabat HCDR1 | NAWMT | SEQ ID |
| | | NO: 6 |
| Kabat HCDR1 | YAWMS | SEQ ID |
| | | NO: 7 |
| Kabat HCDR1 | NYDIN | SEQ ID |
| | | NO: 8 |
| Kabat HCDR1 | SYAMY | SEQ ID |
| | | NO: 9 |
| Kabat HCDR1 | SYAMT | SEQ ID |
| | | NO: 10 |
| Kabat HCDR1 | SYAMS | SEQ ID |
| | | NO: 11 |
| Kabat HCDR1 | TYAMT | SEQ ID |
| | | NO: 12 |
| Kabat HCDR1 | SHAMT | SEQ ID |
| | | NO: 13 |
| Kabat HCDR1 | IYAMT | SEQ ID |
| | | NO: 14 |
| Kabat HCDR1 | SYWMH | SEQ ID |
| | | NO: 15 |
| Kabat HCDR1 | SFWMH | SEQ ID |
| | | NO: 16 |
| Kabat HCDR1 | SAWMS | SEQ ID |
| | | NO: 17 |
| Kabat HCDR1 | NAWMR | SEQ ID |
| | | NO: 18 |
| Kabat HCDR1 | YAWMT | SEQ ID |
| | | NO: 19 |
| Kabat HCDR1 | NAWMN | SEQ ID |
| | | NO: 20 |
| Kabat HCDR1 | TAWMS | SEQ ID |
| | | NO: 21 |
| Chothia HCDR1 | GFTFSNY | SEQ ID |
| | | NO: 22 |
| Chothia HCDR1 | GFRFSSY | SEQ ID |
| | | NO: 23 |
| Chothia HCDR1 | GFPVSSN | SEQ ID |
| | | NO: 24 |
| Chothia HCDR1 | GFTFSYA | SEQ ID |
| | | NO: 25 |
| Chothia HCDR1 | GYTFTNY | SEQ ID |
| | | NO: 26 |
| Chothia HCDR1 | GFTFSSY | SEQ ID |
| | | NO: 27 |
| Chothia HCDR1 | GFTFRNY | SEQ ID |
| | | NO: 28 |
| Chothia HCDR1 | GFTFNTY | SEQ ID |
| | | NO: 29 |
| Chothia HCDR1 | GFTFSSH | SEQ ID |
| | | NO: 30 |
| Chothia HCDR1 | GFTFGNY | SEQ ID |
| | | NO: 31 |
| Chothia HCDR1 | GFTFTNY | SEQ ID |
| | | NO: 32 |
| Chothia HCDR1 | GFTFSIY | SEQ ID |
| | | NO: 33 |
| Chothia HCDR1 | GFTFRSY | SEQ ID |
| | | NO: 34 |
| Chothia HCDR1 | GLTFSNY | SEQ ID |
| | | NO: 35 |
| Chothia HCDR1 | GFTFSSF | SEQ ID |
| | | NO: 36 |
| Chothia HCDR1 | GFTFHNA | SEQ ID |
| | | NO: 37 |
| Chothia HCDR1 | GFTFNSA | SEQ ID |
| | | NO: 38 |
| Chothia HCDR1 | GFIFNNA | SEQ ID |
| | | NO: 39 |
| Chothia HCDR1 | GFTFNYA | SEQ ID |
| | | NO: 40 |
| Chothia HCDR1 | GFTFINA | SEQ ID |
| | | NO: 41 |
| Chothia HCDR1 | GFTFSTA | SEQ ID |
| | | NO: 42 |
| Chothia HCDR1 | GFIFSNA | SEQ ID |
| | | NO: 43 |
| IMGT CDR1 | GFTFSNYA | SEQ ID |
| | | NO: 44 |
| IMGT CDR1 | GFRFSSYA | SEQ ID |
| | | NO: 45 |
| IMGT CDR1 | GFPVSSNY | SEQ ID |
| | | NO: 46 |
| IMGT CDR1 | GFTFSNYW | SEQ ID |
| | | NO: 47 |
| IMGT CDR1 | GFTFSNAW | SEQ ID |
| | | NO: 48 |
| IMGT CDR1 | GFTFNNAW | SEQ ID |
| | | NO: 49 |
| IMGT CDR1 | GFTFSYAW | SEQ ID |
| | | NO: 50 |
| IMGT CDR1 | GYTFTNYD | SEQ ID |
| | | NO: 51 |
| IMGT CDR1 | GFTFSSYA | SEQ ID |
| | | NO: 52 |
| IMGT CDR1 | GFTFRNYA | SEQ ID |
| | | NO: 53 |
| IMGT CDR1 | GFTFNTYA | SEQ ID |
| | | NO: 54 |
| IMGT CDR1 | GFTFSSHA | SEQ ID |
| | | NO: 55 |
| IMGT CDR1 | GFTFGNYA | SEQ ID |
| | | NO: 56 |
| IMGT CDR1 | GFTFTNYA | SEQ ID |
| | | NO: 57 |
| IMGT CDR1 | GFTFSIYA | SEQ ID |
| | | NO: 58 |
| IMGT CDR1 | GFTFRSYW | SEQ ID |
| | | NO: 59 |
| IMGT CDR1 | GLTFSNYW | SEQ ID |
| | | NO: 60 |
| IMGT CDR1 | GFTFSSFW | SEQ ID |
| | | NO: 61 |
| IMGT CDR1 | GFTFSSYW | SEQ ID |
| | | NO: 62 |
| IMGT CDR1 | GFTFHNAW | SEQ ID |
| | | NO: 63 |
| IMGT CDR1 | GFTFNSAW | SEQ ID |
| | | NO: 64 |
| IMGT CDR1 | GFIFNNAW | SEQ ID |
| | | NO: 65 |
| IMGT CDR1 | GFTFNYAW | SEQ ID |
| | | NO: 66 |
| IMGT CDR1 | GFTFINAW | SEQ ID |
| | | NO: 67 |
| IMGT CDR1 | GFTFSTAW | SEQ ID |
| | | NO: 68 |
| IMGT CDR1 | GFIFSNAW | SEQ ID |
| | | NO: 69 |
| Kabat HCDR2 | LISGSGGLTKYADSVKG | SEQ ID |
| | | NO: 70 |
| Kabat HCDR2 | IIYSRDSTHYADSVKG | SEQ ID |
| | | NO: 71 |
| Kabat HCDR2 | RINSDGISTRYADSVKG | SEQ ID |
| | | NO: 72 |
| Kabat HCDR2 | RIKSNSYGGTTDYAAPAIG | SEQ ID |
| | | NO: 73 |
| Kabat HCDR2 | RIKSISYGGTTDSAAPVKG | SEQ ID |
| | | NO: 74 |
| Kabat HCDR2 | RIKSETDGGTTDYAAPVKG | SEQ ID |
| | | NO: 75 |
| Kabat HCDR2 | WMNPHSGQTGYAQKFQG | SEQ ID |
| | | NO: 76 |
| Kabat HCDR2 | LITGSGGSTHYADSVKG | SEQ ID |
| | | NO: 77 |
| Kabat HCDR2 | LISGSGGITKYADSVKG | SEQ ID |
| | | NO: 78 |
| Kabat HCDR2 | LISGSGGITHYADSVKG | SEQ ID |
| | | NO: 79 |
| Kabat HCDR2 | LISGSGGVTHYADSVKG | SEQ ID |
| | | NO: 80 |
| Kabat HCDR2 | LISGSGAITHYADSVKG | SEQ ID |
| | | NO: 81 |
| Kabat HCDR2 | LISGSGGLTNYADSVKG | SEQ ID |
| | | NO: 82 |
| Kabat HCDR2 | LISGSGGSTHYADSVKG | SEQ ID |
| | | NO: 83 |
| Kabat HCDR2 | LISGSGGLTKYADSVRG | SEQ ID |
| | | NO: 84 |
| Kabat HCDR2 | LISGSGSLTKYADSVKG | SEQ ID |
| | | NO: 85 |
| Kabat HCDR2 | LISGGGGLTYYADSVKG | SEQ ID |
| | | NO: 86 |
| Kabat HCDR2 | LISGSGGLTYYADSVKG | SEQ ID |
| | | NO: 87 |
| Kabat HCDR2 | RIDNNGINTRYADSVKG | SEQ ID |
| | | NO: 88 |
| Kabat HCDR2 | RISSDGISTRYADSVKG | SEQ ID |
| | | NO: 89 |
| Kabat HCDR2 | RINNDGISTRYADSVKG | SEQ ID |
| | | NO: 90 |
| Kabat HCDR2 | RINSDGFSTRYADSVKG | SEQ ID |
| | | NO: 91 |
| Kabat HCDR2 | RIKGVTAGGTADYAAPVKG | SEQ ID |
| | | NO: 92 |
| Kabat HCDR2 | RVKSVTAGGTTDYAAPVKG | SEQ ID |
| | | NO: 93 |
| Kabat HCDR2 | RIKSKTDGGTTDYAAPVKG | SEQ ID |
| | | NO: 94 |
| Kabat HCDR2 | RIKSVTTGGTTDYAAPVKG | SEQ ID |
| | | NO: 95 |
| Kabat HCDR2 | RIKSVTAGGTTDYAAPVKG | SEQ ID |
| | | NO: 96 |
| Kabat HCDR2 | RIKSVTAGGTTDFPAPVKG | SEQ ID |
| | | NO: 97 |
| Kabat HCDR2 | HIKSETDGGTTDYAAPVKG | SEQ ID |
| | | NO: 98 |
| Kabat HCDR2 | RIKRKTDGGTTDYAAPVQG | SEQ ID |
| | | NO: 99 |
| Kabat HCDR2 | RIKSLTDGGTTDYAAPVKG | SEQ ID |
| | | NO: 100 |
| Kabat HCDR2 | RIKRKTDGGTREYAAPVKG | SEQ ID |
| | | NO: 101 |
| Kabat HCDR2 | RIKRNTDGGTTDYAAPVKG | SEQ ID |
| | | NO: 102 |
| Kabat HCDR2 | RIKTNTDGGTTDYAAPVKG | SEQ ID |
| | | NO: 103 |
| Kabat HCDR2 | RIKSKTYGGTTDYDAPVKG | SEQ ID |
| | | NO: 104 |
| Kabat HCDR2 | RIKSQTYGGTTDYAAPVKG | SEQ ID |
| | | NO: 105 |
| Kabat HCDR2 | RIKSKTYGGTTDYAAPVKG | SEQ ID |
| | | NO: 106 |
| Kabat HCDR2 | RIKRKTDGGTRDYAAPVKG | SEQ ID |
| | | NO: 107 |
| Kabat HCDR2 | RIKRETDGGTTDYASPVKG | SEQ ID |
| | | NO: 108 |
| Kabat HCDR2 | RIKRKSDGGTTDYAAPVKG | SEQ ID |
| | | NO: 109 |
| Kabat HCDR2 | RIKSEIDGGTTDYAAPVKG | SEQ ID |
| | | NO: 110 |
| Chothia HCDR2 | SGSGGL | SEQ ID |
| | | NO: 111 |
| Chothia HCDR2 | YSRDS | SEQ ID |
| | | NO: 112 |
| Chothia HCDR2 | NSDGIS | SEQ ID |
| | | NO: 113 |
| Chothia HCDR2 | KSNSYGGT | SEQ ID |
| | | NO: 114 |
| Chothia HCDR2 | KSISYGGT | SEQ ID |
| | | NO: 115 |
| Chothia HCDR2 | KSETDGGT | SEQ ID |
| | | NO: 116 |
| Chothia HCDR2 | NPHSGQ | SEQ ID |
| | | NO: 117 |
| Chothia HCDR2 | TGSGGS | SEQ ID |
| | | NO: 118 |
| Chothia HCDR2 | SGSGGI | SEQ ID |
| | | NO: 119 |
| Chothia HCDR2 | SGSGGV | SEQ ID |
| | | NO: 120 |
| Chothia HCDR2 | SGSGAI | SEQ ID |
| | | NO: 121 |
| Chothia HCDR2 | SGSGGS | SEQ ID |
| | | NO: 122 |
| Chothia HCDR2 | SGSGSL | SEQ ID |
| | | NO: 123 |
| Chothia HCDR2 | SGGGGL | SEQ ID |
| | | NO: 124 |
| Chothia HCDR2 | DNNGIN | SEQ ID |
| | | NO: 125 |
| Chothia HCDR2 | SSDGIS | SEQ ID |
| | | NO: 126 |
| Chothia HCDR2 | NNDGIS | SEQ ID |
| | | NO: 127 |
| Chothia HCDR2 | NSDGFS | SEQ ID |
| | | NO: 128 |
| Chothia HCDR2 | KGVTAGGT | SEQ ID |
| | | NO: 129 |
| Chothia HCDR2 | KSVTAGGT | SEQ ID |
| | | NO: 130 |
| Chothia HCDR2 | KSKTDGGT | SEQ ID |
| | | NO: 131 |
| Chothia HCDR2 | KSVTTGGT | SEQ ID |
| | | NO: 132 |
| Chothia HCDR2 | KRKTDGGT | SEQ ID |
| | | NO: 133 |
| Chothia HCDR2 | KSLTDGGT | SEQ ID |
| | | NO: 134 |
| Chothia HCDR2 | KRNTDGGT | SEQ ID |
| | | NO: 135 |
| Chothia HCDR2 | KRNTDGGT | SEQ ID |
| | | NO: 136 |
| Chothia HCDR2 | KSKTYGGT | SEQ ID |
| | | NO: 137 |
| Chothia HCDR2 | KSQTYGGT | SEQ ID |
| | | NO: 138 |
| Chothia HCDR2 | KRETDGGT | SEQ ID |
| | | NO: 139 |
| Chothia HCDR2 | KRKSDGGT | SEQ ID |
| | | NO: 140 |
| Chothia HCDR2 | KSEIDGGT | SEQ ID |
| | | NO: 141 |
| IMGT HCDR2 | ISGSGGLT | SEQ ID |
| | | NO: 142 |
| IMGT HCDR2 | IYSRDST | SEQ ID |
| | | NO: 143 |
| IMGT HCDR2 | INSDGIST | SEQ ID |
| | | NO: 144 |
| IMGT HCDR2 | IKSNSYGGTT | SEQ ID |
| | | NO: 145 |
| IMGT HCDR2 | IKSISYGGTT | SEQ ID |
| | | NO: 146 |
| IMGT HCDR2 | IKSETDGGTT | SEQ ID |
| | | NO: 147 |
| IMGT HCDR2 | MNPHSGQT | SEQ ID |
| | | NO: 148 |
| IMGT HCDR2 | ITGSGGST | SEQ ID |
| | | NO: 149 |
| IMGT HCDR2 | ISGSGGIT | SEQ ID |
| | | NO: 150 |
| IMGT HCDR2 | ISGSGGVT | SEQ ID |
| | | NO: 151 |
| IMGT HCDR2 | ISGSGAIT | SEQ ID |
| | | NO: 152 |
| IMGT HCDR2 | ISGSGGST | SEQ ID |
| | | NO: 153 |
| IMGT HCDR2 | ISGSGSLT | SEQ ID |
| | | NO: 154 |
| IMGT HCDR2 | ISGGGGLT | SEQ ID |
| | | NO: 155 |
| IMGT HCDR2 | IDNNGINT | SEQ ID |
| | | NO: 156 |
| IMGT HCDR2 | ISSDGIST | SEQ ID |
| | | NO: 157 |
| IMGT HCDR2 | INNDGIST | SEQ ID |
| | | NO: 158 |
| IMGT HCDR2 | INSDGFST | SEQ ID |
| | | NO: 159 |
| IMGT HCDR2 | IKGVTAGGTA | SEQ ID |
| | | NO: 160 |
| IMGT HCDR2 | VKSVTAGGTT | SEQ ID |
| | | NO: 161 |
| IMGT HCDR2 | IKSKTDGGTT | SEQ ID |
| | | NO: 162 |
| IMGT HCDR2 | IKSVTTGGTT | SEQ ID |
| | | NO: 163 |
| IMGT HCDR2 | IKSVTAGGTT | SEQ ID |
| | | NO: 164 |
| IMGT HCDR2 | IKRKTDGGTT | SEQ ID |
| | | NO: 165 |
| IMGT HCDR2 | IKSLTDGGTT | SEQ ID |
| | | NO: 166 |
| IMGT HCDR2 | IKRKTDGGTR | SEQ ID |
| | | NO: 167 |
| IMGT HCDR2 | IKRNTDGGTT | SEQ ID |
| | | NO: 168 |
| IMGT HCDR2 | IKTNTDGGTT | SEQ ID |
| | | NO: 169 |
| IMGT HCDR2 | IKSKTYGGTT | SEQ ID |
| | | NO: 170 |
| IMGT HCDR2 | IKSQTYGGTT | SEQ ID |
| | | NO: 171 |
| IMGT HCDR2 | IKRETDGGTT | SEQ ID |
| | | NO: 172 |
| IMGT HCDR2 | IKRKSDGGTT | SEQ ID |
| | | NO: 173 |
| IMGT HCDR2 | IKSEIDGGTT | SEQ ID |
| | | NO: 174 |
| Kabat HCDR3 | DEGLTTGEY | SEQ ID |
| | | NO: 175 |
| Kabat HCDR3 | ERWEPSFHY | SEQ ID |
| | | NO: 176 |
| Kabat HCDR3 | TLTYYYGAGSDY | SEQ ID |
| | | NO: 177 |
| Kabat HCDR3 | DFQLELPDY | SEQ ID |
| | | NO: 178 |
| Kabat HCDR3 | DHPKFGFDF | SEQ ID |
| | | NO: 179 |
| Kabat HCDR3 | GSVWKYGGSLDF | SEQ ID |
| | | NO: 180 |
| Kabat HCDR3 | GSSWNVYFFDF | SEQ ID |
| | | NO: 181 |
| Kabat HCDR3 | EGYDLWSGDSSNWLDP | SEQ ID |
| | | NO: 182 |
| Kabat HCDR3 | EGYDLWSGESSNWLDP | SEQ ID |
| | | NO: 183 |
| Kabat HCDR3 | EGYDLWSGHSSNWLDP | SEQ ID |
| | | NO: 184 |
| Kabat HCDR3 | EGYDLWSGYSSNWLDP | SEQ ID |
| | | NO: 185 |
| Kabat HCDR3 | DEGLTTGEF | SEQ ID |
| | | NO: 186 |
| Kabat HCDR3 | DEGLTTGES | SEQ ID |
| | | NO: 187 |
| Kabat HCDR3 | EEGLTTGEY | SEQ ID |
| | | NO: 188 |
| Kabat HCDR3 | DEGLTSGEY | SEQ ID |
| | | NO: 189 |
| Kabat HCDR3 | DFQLELRDY | SEQ ID |
| | | NO: 190 |
| Kabat HCDR3 | DFQLELRDF | SEQ ID |
| | | NO: 191 |
| Kabat HCDR3 | DHPKFGFDY | SEQ ID |
| | | NO: 192 |
| Kabat HCDR3 | GGVWNYGGSLDF | SEQ ID |
| | | NO: 193 |
| Kabat HCDR3 | GGVWKYGGSLDF | SEQ ID |
| | | NO: 194 |
| Kabat HCDR3 | GSVWNYGGSLDF | SEQ ID |
| | | NO: 195 |
| Kabat HCDR3 | GSVWNYGGSLDY | SEQ ID |
| | | NO: 196 |
| Kabat HCDR3 | GSVWKYGGSLDY | SEQ ID |
| | | NO: 197 |
| Kabat HCDR3 | GSVWRYGGSLDF | SEQ ID |
| | | NO: 198 |
| Kabat HCDR3 | GSSWHVYFFDF | SEQ ID |
| | | NO: 199 |
| Kabat HCDR3 | GSSWSLYYFDF | SEQ ID |
| | | NO: 200 |
| Kabat HCDR3 | GSSWYVYYFDY | SEQ ID |
| | | NO: 201 |
| Kabat HCDR3 | GSSWYVYYFDF | SEQ ID |
| | | NO: 202 |
| Chothia HCDR3 | EGLTTGE | SEQ ID |
| | | NO: 203 |
| Chothia HCDR3 | RWEPSFH | SEQ ID |
| | | NO: 204 |
| Chothia HCDR3 | LTYYYGAGSD | SEQ ID |
| | | NO: 205 |
| Chothia HCDR3 | FQLELPD | SEQ ID |
| | | NO: 206 |
| Chothia HCDR3 | HPKFGFD | SEQ ID |
| | | NO: 207 |
| Chothia HCDR3 | SVWKYGGSLD | SEQ ID |
| | | NO: 208 |
| Chothia HCDR3 | SSWNVYFFD | SEQ ID |
| | | NO: 209 |
| Chothia HCDR3 | GYDLWSGDSSNWLD | SEQ ID |
| | | NO: 210 |
| Chothia HCDR3 | GYDLWSGESSNWLD | SEQ ID |
| | | NO: 211 |
| Chothia HCDR3 | GYDLWSGHSSNWLD | SEQ ID |
| | | NO: 212 |
| Chothia HCDR3 | GYDLWSGYSSNWLD | SEQ ID |
| | | NO: 213 |
| Chothia HCDR3 | EGLTSGE | SEQ ID |
| | | NO: 214 |
| Chothia HCDR3 | FQLELRD | SEQ ID |
| | | NO: 215 |
| Chothia HCDR3 | GVWNYGGSLD | SEQ ID |
| | | NO: 216 |
| Chothia HCDR3 | GVWKYGGSLD | SEQ ID |
| | | NO: 217 |
| Chothia HCDR3 | SVWNYGGSLD | SEQ ID |
| | | NO: 218 |
| Chothia HCDR3 | SVWRYGGSLD | SEQ ID |
| | | NO: 219 |
| Chothia HCDR3 | SSWHVYFFD | SEQ ID |
| | | NO: 220 |
| Chothia HCDR3 | SSWSLYYFD | SEQ ID |
| | | NO: 221 |
| Chothia HCDR3 | SSWYVYYFD | SEQ ID |
| | | NO: 222 |
| IMGT HCDR3 | VKDEGLTTGEY | SEQ ID |
| | | NO: 223 |
| IMGT HCDR3 | AKERWEPSFHY | SEQ ID |
| | | NO: 224 |
| IMGT HCDR3 | AGTLTYYYGAGSDY | SEQ ID |
| | | NO: 225 |
| IMGT HCDR3 | VRDFQLELPDY | SEQ ID |
| | | NO: 226 |
| IMGT HCDR3 | TTDHPKFGFDF | SEQ ID |
| | | NO: 227 |
| IMGT HCDR3 | STGSVWKYGGSLDF | SEQ ID |
| | | NO: 228 |
| IMGT HCDR3 | TTGSSWNVYFFDF | SEQ ID |
| | | NO: 229 |
| IMGT HCDR3 | AREGYDLWSGDSSNWLDP | SEQ ID |
| | | NO: 230 |
| IMGT HCDR3 | AREGYDLWSGESSNWLDP | SEQ ID |
| | | NO: 231 |
| IMGT HCDR3 | AREGYDLWSGHSSNWLDP | SEQ ID |
| | | NO: 232 |
| IMGT HCDR3 | AREGYDLWSGYSSNWLDP | SEQ ID |
| | | NO: 233 |
| IMGT HCDR3 | AKDEGLTTGEY | SEQ ID |
| | | NO: 234 |
| IMGT HCDR3 | VKDEGLTTGEF | SEQ ID |
| | | NO: 235 |
| IMGT HCDR3 | VKDEGLTTGES | SEQ ID |
| | | NO: 236 |
| IMGT HCDR3 | AKEEGLTTGEY | SEQ ID |
| | | NO: 237 |
| IMGT HCDR3 | AKDEGLTSGEY | SEQ ID |
| | | NO: 238 |
| IMGT HCDR3 | VKEEGLTTGEY | SEQ ID |
| | | NO: 239 |
| IMGT HCDR3 | VRDFQLELRDY | SEQ ID |
| | | NO: 240 |
| IMGT HCDR3 | VSDFQLELPDY | SEQ ID |
| | | NO: 241 |
| IMGT HCDR3 | VRDFQLELRDF | SEQ ID |
| | | NO: 242 |
| IMGT HCDR3 | TTDHPKFGFDY | SEQ ID |
| | | NO: 243 |
| IMGT HCDR3 | TTGGVWNYGGSLDF | SEQ ID |
| | | NO: 244 |
| IMGT HCDR3 | ITGGVWNYGGSLDF | SEQ ID |
| | | NO: 245 |
| IMGT HCDR3 | TTGGVWKYGGSLDF | SEQ ID |
| | | NO: 246 |
| IMGT HCDR3 | ATGGVWKYGGSLDF | SEQ ID |
| | | NO: 247 |
| IMGT HCDR3 | TTGSVWKYGGSLDF | SEQ ID |
| | | NO: 248 |
| IMGT HCDR3 | STGSVWNYGGSLDF | SEQ ID |
| | | NO: 249 |
| IMGT HCDR3 | TTGSVWNYGGSLDY | SEQ ID |
| | | NO: 250 |
| IMGT HCDR3 | TTGSVWKYGGSLDY | SEQ ID |
| | | NO: 251 |
| IMGT HCDR3 | TTGSVWRYGGSLDF | SEQ ID |
| | | NO: 252 |
| IMGT HCDR3 | TTGSVWNYGGSLDF | SEQ ID |
| | | NO: 253 |
| IMGT HCDR3 | TTGSSWHVYFFDF | SEQ ID |
| | | NO: 254 |
| IMGT HCDR3 | TTGSSWSLYYFDF | SEQ ID |
| | | NO: 255 |
| IMGT HCDR3 | TTGSSWYVYYFDY | SEQ ID |
| | | NO: 256 |
| IMGT HCDR3 | TTGSSWYVYYFDF | SEQ ID |
| | | NO: 257 |
| Kabat LCDR1 | RASQDIRNDLA | SEQ ID |
| | | NO: 258 |
| Kabat LCDR1 | QASQGITNYLN | SEQ ID |
| | | NO: 259 |
| Kabat LCDR1 | RASQGIRNDLG | SEQ ID |
| | | NO: 260 |
| Kabat LCDR1 | RASQGIRDDLA | SEQ ID |
| | | NO: 261 |
| Kabat LCDR1 | KSSQSVFYSSNNKNYLA | SEQ ID |
| | | NO: 262 |
| Kabat LCDR1 | RASQGISRFLN | SEQ ID |
| | | NO: 263 |
| Kabat LCDR1 | RASQSISNYLN | SEQ ID |
| | | NO: 264 |
| Kabat LCDR1 | RASQGISSYLA | SEQ ID |
| | | NO: 265 |
| Kabat LCDR1 | RASQDIRNDLD | SEQ ID |
| | | NO: 266 |
| Kabat LCDR1 | RASQAIRNDLG | SEQ ID |
| | | NO: 267 |
| Kabat LCDR1 | RTSQGIRNDLA | SEQ ID |
| | | NO: 268 |
| Kabat LCDR1 | RASQGIRNDLA | SEQ ID |
| | | NO: 269 |
| Kabat LCDR1 | RASQGIRNNLG | SEQ ID |
| | | NO: 270 |
| Kabat LCDR1 | RASQAIRDDLA | SEQ ID |
| | | NO: 271 |
| Kabat LCDR1 | KSSQSVFYTSNSKHYLN | SEQ ID |
| | | NO: 272 |
| Kabat LCDR1 | KSSQSVLYSSNNKNYLA | SEQ ID |
| | | NO: 273 |
| Kabat LCDR1 | KSSQSVFYTSNNKNYLN | SEQ ID |
| | | NO: 274 |
| Kabat LCDR1 | KSSQSVFYTSSNKHYLN | SEQ ID |
| | | NO: 275 |
| Kabat LCDR1 | RASQSITRYLN | SEQ ID |
| | | NO: 276 |
| Kabat LCDR1 | RASQFITRYLN | SEQ ID |
| | | NO: 277 |
| Kabat LCDR1 | RASQGISRYLN | SEQ ID |
| | | NO: 278 |
| Kabat LCDR1 | RASQSITRSLN | SEQ ID |
| | | NO: 279 |
| Kabat LCDR1 | RASQSISRYLN | SEQ ID |
| | | NO: 280 |
| Kabat LCDR1 | RASQNISRYLN | SEQ ID |
| | | NO: 281 |
| Kabat LCDR1 | RASQIISRYLN | SEQ ID |
| | | NO: 282 |
| Kabat LCDR1 | RASQSMSSHLN | SEQ ID |
| | | NO: 283 |
| Kabat LCDR1 | RASQSISTYLN | SEQ ID |
| | | NO: 284 |
| Kabat LCDR1 | RASQSISSYLN | SEQ ID |
| | | NO: 285 |
| Chothia LCDR1 | SQDIRND | SEQ ID |
| | | NO: 286 |
| Chothia LCDR1 | SQGITNY | SEQ ID |
| | | NO: 287 |
| Chothia LCDR1 | SQGIRND | SEQ ID |
| | | NO: 288 |
| Chothia LCDR1 | SQGIRDD | SEQ ID |
| | | NO: 289 |
| Chothia LCDR1 | SQSVFYSSNNKNY | SEQ ID |
| | | NO: 290 |
| Chothia LCDR1 | SQGISRF | SEQ ID |
| | | NO: 291 |
| Chothia LCDR1 | SQSISNY | SEQ ID |
| | | NO: 292 |
| Chothia LCDR1 | SQGISSY | SEQ ID |
| | | NO: 293 |
| Chothia LCDR1 | SQAIRND | SEQ ID |
| | | NO: 294 |
| Chothia LCDR1 | SQGIRNN | SEQ ID |
| | | NO: 295 |
| Chothia LCDR1 | SQAIRDD | SEQ ID |
| | | NO: 296 |
| Chothia LCDR1 | SQSVFYTSNSKHY | SEQ ID |
| | | NO: 297 |
| Chothia LCDR1 | SQSVLYSSNNKNY | SEQ ID |
| | | NO: 298 |
| Chothia LCDR1 | SQSVFYTSNNKNY | SEQ ID |
| | | NO: 299 |
| Chothia LCDR1 | SQSVFYTSSNKHY | SEQ ID |
| | | NO: 300 |
| Chothia LCDR1 | SQSITRY | SEQ ID |
| | | NO: 301 |
| Chothia LCDR1 | SQFITRY | SEQ ID |
| | | NO: 302 |
| Chothia LCDR1 | SQGISRY | SEQ ID |
| | | NO: 303 |
| Chothia LCDR1 | SQSITRS | SEQ ID |
| | | NO: 304 |
| Chothia LCDR1 | SQSISRY | SEQ ID |
| | | NO: 305 |
| Chothia LCDR1 | SQNISRY | SEQ ID |
| | | NO: 306 |
| Chothia LCDR1 | SQIISRY | SEQ ID |
| | | NO: 307 |
| Chothia LCDR1 | SQSMSSH | SEQ ID |
| | | NO: 308 |
| Chothia LCDR1 | SQSISTY | SEQ ID |
| | | NO: 309 |
| Chothia LCDR1 | SQSISSY | SEQ ID |
| | | NO: 310 |
| IMGT LCDR1 | QDIRND | SEQ ID |
| | | NO: 311 |
| IMGT LCDR1 | QGITNY | SEQ ID |
| | | NO: 312 |
| IMGT LCDR1 | QGIRND | SEQ ID |
| | | NO: 313 |
| IMGT LCDR1 | QGIRDD | SEQ ID |
| | | NO: 314 |
| IMGT LCDR1 | QSVFYSSNNKNY | SEQ ID |
| | | NO: 315 |
| IMGT LCDR1 | QGISRF | SEQ ID |
| | | NO: 316 |
| IMGT LCDR1 | QSISNY | SEQ ID |
| | | NO: 317 |
| IMGT LCDR1 | QGISSY | SEQ ID |
| | | NO: 318 |
| IMGT LCDR1 | QAIRND | SEQ ID |
| | | NO: 319 |
| IMGT LCDR1 | QAIRDD | SEQ ID |
| | | NO: 320 |
| IMGT LCDR1 | QSVFYTSNSKHY | SEQ ID |
| | | NO: 321 |
| IMGT LCDR1 | QSVLYSSNNKNY | SEQ ID |
| | | NO: 322 |
| IMGT LCDR1 | QSVFYTSNNKNY | SEQ ID |
| | | NO: 323 |
| IMGT LCDR1 | QSVFYTSSNKHY | SEQ ID |
| | | NO: 324 |
| IMGT LCDR1 | QSITRY | SEQ ID |
| | | NO: 325 |
| IMGT LCDR1 | QFITRY | SEQ ID |
| | | NO: 326 |
| IMGT LCDR1 | QGISRY | SEQ ID |
| | | NO: 327 |
| IMGT LCDR1 | QSITRS | SEQ ID |
| | | NO: 328 |
| IMGT LCDR1 | QSISRY | SEQ ID |
| | | NO: 329 |
| IMGT LCDR1 | QNISRY | SEQ ID |
| | | NO: 330 |
| IMGT LCDR1 | QIISRY | SEQ ID |
| | | NO: 331 |
| IMGT LCDR1 | QSMSSH | SEQ ID |
| | | NO: 332 |
| IMGT LCDR1 | QSISTY | SEQ ID |
| | | NO: 333 |
| IMGT LCDR1 | QSISSY | SEQ ID |
| | | NO: 334 |
| Kabat LCDR2 | ASSSLQS | SEQ ID |
| | | NO: 335 |
| Kabat LCDR2 | DASNLET | SEQ ID |
| | | NO: 336 |
| Kabat LCDR2 | AASSLQS | SEQ ID |
| | | NO: 337 |
| Kabat LCDR2 | WASTRES | SEQ ID |
| | | NO: 338 |
| Kabat LCDR2 | AASTLQN | SEQ ID |
| | | NO: 339 |
| Kabat LCDR2 | AAYRLQT | SEQ ID |
| | | NO: 340 |
| Kabat LCDR2 | AASTLQS | SEQ ID |
| | | NO: 341 |
| Kabat LCDR2 | VVSSFQS | SEQ ID |
| | | NO: 342 |
| Kabat LCDR2 | ATSSLQS | SEQ ID |
| | | NO: 343 |
| Kabat LCDR2 | ATSNLQS | SEQ ID |
| | | NO: 344 |
| Kabat LCDR2 | AASSFQS | SEQ ID |
| | | NO: 345 |
| Kabat LCDR2 | AASSLKS | SEQ ID |
| | | NO: 346 |
| Kabat LCDR2 | ATSSFQS | SEQ ID |
| | | NO: 347 |
| Kabat LCDR2 | AVSSLQS | SEQ ID |
| | | NO: 348 |
| Kabat LCDR2 | ATSILQS | SEQ ID |
| | | NO: 349 |
| Kabat LCDR2 | AASSLQN | SEQ ID |
| | | NO: 350 |
| Kabat LCDR2 | AASILQS | SEQ ID |
| | | NO: 351 |
| Kabat LCDR2 | GASTLQS | SEQ ID |
| | | NO: 352 |
| Kabat LCDR2 | AASRLQS | SEQ ID |
| | | NO: 353 |
| Kabat LCDR2 | GASSLES | SEQ ID |
| | | NO: 354 |
| Kabat LCDR2 | GASSLQS | SEQ ID |
| | | NO: 355 |
| Kabat LCDR2 | AASRLES | SEQ ID |
| | | NO: 356 |
| Kabat LCDR2 | AASNLQS | SEQ ID |
| | | NO: 357 |
| Kabat LCDR2 | AASSLQS | SEQ ID |
| | | NO: 358 |
| Chothia and | ASS | |
| IMGT LCDR2 | | |
| Chothia and | DAS | |
| IMGT LCDR2 | | |
| Chothia and | AAS | |
| IMGT LCDR2 | | |
| Chothia and | WAS | |
| IMGT LCDR2 | | |
| Chothia and | AAY | |
| IMGT LCDR2 | | |
| Chothia and | VVS | |
| IMGT LCDR2 | | |
| Chothia and | ATS | |
| IMGT LCDR2 | | |
| Chothia and | AVS | |
| IMGT LCDR2 | | |
| Chothia and | GAS | |
| IMGT LCDR2 | | |
| Kabat LCDR3 | LQHNNYPFT | SEQ ID |
| | | NO: 368 |
| Kabat LCDR3 | QQYENLPYT | SEQ ID |
| | | NO: 369 |
| Kabat LCDR3 | LQHNSYPWS | SEQ ID |
| | | NO: 370 |
| Kabat LCDR3 | LQHKSNPFT | SEQ ID |
| | | NO: 371 |
| Kabat LCDR3 | QQHYDIPRT | SEQ ID |
| | | NO: 372 |
| Kabat LCDR3 | QQTYSTPLT | SEQ ID |
| | | NO: 373 |
| Kabat LCDR3 | QQSYSSPLT | SEQ ID |
| | | NO: 374 |
| Kabat LCDR3 | QQLNSYPRT | SEQ ID |
| | | NO: 375 |
| Kabat LCDR3 | QQLNNYPRT | SEQ ID |
| | | NO: 376 |
| Kabat LCDR3 | QQLQSYPRT | SEQ ID |
| | | NO: 377 |
| Kabat LCDR3 | LQHNSYPFT | SEQ ID |
| | | NO: 378 |
| Kabat LCDR3 | LQHNTYPFS | SEQ ID |
| | | NO: 379 |
| Kabat LCDR3 | LQHSSYPFT | SEQ ID |
| | | NO: 380 |
| Kabat LCDR3 | LQHNTYPFT | SEQ ID |
| | | NO: 381 |
| Kabat LCDR3 | LQHNSNPFT | SEQ ID |
| | | NO: 382 |
| Kabat LCDR3 | LQHYSNPFT | SEQ ID |
| | | NO: 383 |
| Kabat LCDR3 | QQYYSIPRT | SEQ ID |
| | | NO: 384 |
| Kabat LCDR3 | QQHYSTPRT | SEQ ID |
| | | NO: 385 |
| Kabat LCDR3 | QQYYSTPRT | SEQ ID |
| | | NO: 386 |
| Kabat LCDR3 | QQSYSTPLT | SEQ ID |
| | | NO: 387 |
| Kabat LCDR3 | QQSYNTPLT | SEQ ID |
| | | NO: 388 |
| Kabat LCDR3 | QQSYSTPVT | SEQ ID |
| | | NO: 389 |
| Kabat LCDR3 | QQSYRTSLT | SEQ ID |
| | | NO: 390 |
| Kabat LCDR3 | QQSYGTPLT | SEQ ID |
| | | NO: 391 |
| Kabat LCDR3 | QQSYSTPFT | SEQ ID |
| | | NO: 392 |
| Kabat LCDR3 | QQSYRSPFT | SEQ ID |
| | | NO: 393 |
| Chothia and | HNNYPF | SEQ ID |
| IMGT LCDR3 | | NO: 394 |
| Chothia and | YENLPY | SEQ ID |
| IMGT LCDR3 | | NO: 395 |
| Chothia and | HNSYPW | SEQ ID |
| IMGT LCDR3 | | NO: 396 |
| Chothia and | HKSNPF | SEQ ID |
| IMGT LCDR3 | | NO: 397 |
| Chothia and | HYDIPR | SEQ ID |
| IMGT LCDR3 | | NO: 398 |
| Chothia and | TYSTPL | SEQ ID |
| IMGT LCDR3 | | NO: 399 |
| Chothia and | SYSSPL | SEQ ID |
| IMGT LCDR3 | | NO: 400 |
| Chothia and | LNSYPR | SEQ ID |
| IMGT LCDR3 | | NO: 401 |
| Chothia and | LNNYPR | SEQ ID |
| IMGT LCDR3 | | NO: 402 |
| Chothia and | LQSYPR | SEQ ID |
| IMGT LCDR3 | | NO: 403 |
| Chothia and | HNSYPF | SEQ ID |
| IMGT LCDR3 | | NO: 404 |
| Chothia and | HNTYPF | SEQ ID |
| IMGT LCDR3 | | NO: 405 |
| Chothia and | HSSYPF | SEQ ID |
| IMGT LCDR3 | | NO: 406 |
| Chothia and | HNSNPF | SEQ ID |
| IMGT LCDR3 | | NO: 407 |
| Chothia and | HYSNPF | SEQ ID |
| IMGT LCDR3 | | NO: 408 |
| Chothia and | YYSIPR | SEQ ID |
| IMGT LCDR3 | | NO: 409 |
| Chothia and | HYSTPR | SEQ ID |
| IMGT LCDR3 | | NO: 410 |
| Chothia and | YYSTPR | SEQ ID |
| IMGT LCDR3 | | NO: 411 |
| Chothia and | SYSTPL | SEQ ID |
| IMGT LCDR3 | | NO: 412 |
| Chothia and | SYNTPL | SEQ ID |
| IMGT LCDR3 | | NO: 413 |
| Chothia and | SYSTPV | SEQ ID |
| IMGT LCDR3 | | NO: 414 |
| Chothia and | SYRTSL | SEQ ID |
| IMGT LCDR3 | | NO: 415 |
| Chothia and | SYGTPL | SEQ ID |
| IMGT LCDR3 | | NO: 416 |
| Chothia and | SYSTPF | SEQ ID |
| IMGT LCDR3 | | NO: 417 |
| Chothia and | SYRSPF | SEQ ID |
| IMGT LCDR3 | | NO: 418 |
| Heavy Chain | EVQVVESGGGLVQPGGSLRLSCAASGFTFSNYAMTWVRQSP | SEQ ID |
| Variable Domain | GKGLEWVSLISGSGGLTKYADSVKGRFTISRDNSKKTLFLQM | NO: 419 |
| 1 | NSLRAEDTAAYYCVKDEGLTTGEYWGQGTQVTVSS | |
| Heavy Chain | KVQLVESGGGLVQPGGSLRLSCAASGFRFSSYAMNWVRQAP | SEQ ID |
| Variable Domain | GKGLEWVSLISGSGGLTKYADSVKGRFTISRDNSRNTLYLQM | NO: 420 |
| 2 | ISLRAEDTAVYFCAKERWEPSFHYWGQGTLVTVSS | |
| Heavy Chain | EVQVVESGGGLIQPGGSLRISCAASGFPVSSNYMSWVRQAPG | SEQ ID |
| Variable Domain | KGLDWVSIIYSRDSTHYADSVKGRFTMSRDNSKNTVYLQMN | NO: 421 |
| 3 | SLRAEDTAVYYCAGTLTYYYGAGSDYWGQGTLVTVSS | |
| Heavy Chain | EVQLVESGGGLVQPGGSLRLSCAASGFTFSNYWMHWVRQA | SEQ ID |
| Variable Domain | PGKGLVWVSRINSDGISTRYADSVKGRFTISRDNAKNTLYLQ | NO: 422 |
| 4 | MKSLRAEDTAVYYCVRDFQLELPDYWGQGTLVTVSS | |
| Heavy Chain | EVQLVESGGGFVKPGGSLRLSCAASGFTFSNAWMSWVRQAP | SEQ ID |
| Variable Domain | GKGLEWVGRIKSNSYGGTTDYAAPAIGRFTISRDGSKSTLYL | NO: 423 |
| 5 | QMNSLKTEDTAVYYCTTDHPKFGFDFWGQGTLVTVSS | |
| Heavy Chain | EVQLVESGGGLVTPGGSLRISCAASGFTFNNAWMTWVRQAP | SEQ ID |
| Variable Domain | GKGLEWVGRIKSISYGGTTDSAAPVKGRFTISRDDSENTLYL | NO: 424 |
| 6 | QMSSLKTEDTAVYYCSTGSVWKYGGSLDFWGQGTLVTVSS | |
| Heavy Chain | EVQLVESGGGLVKPGGSLRLSCAASGFTFSYAWMSWVRQAP | SEQ ID |
| Variable Domain | GRGLEWVGRIKSETDGGTTDYAAPVKGRFTISRDHSENTLFL | NO: 425 |
| 7 | QMNSLKTEDTAVYYCTTGSSWNVYFFDFWGQGTLVTVSS | |
| Heavy Chain | QVQLVQSGAEVKKPGASVKVSCKASGYTFTNYDINWVRQA | SEQ ID |
| Variable Domain | TGQGLEWMGWMNPHSGQTGYAQKFQGRVTMTRNTAISTA | NO: 426 |
| 8 | YMELSSLRSEDTAVYYCAREGYDLWSGDSSNWLDPWGQGT | |
| | LVTVSA | |
| Heavy Chain | QVQLVQSGAEVKKPGASVKVSCKASGYTFTNYDINWVRQA | SEQ ID |
| Variable Domain | TGQGLEWMGWMNPHSGQTGYAQKFQGRVTMTRNTAISTA | NO: 427 |
| 9 | YMELSSLRSEDTAVYYCAREGYDLWSGESSNWLDPWGQGT | |
| | LVTVSA | |
| Heavy Chain | QVQLVQSGAEVKKPGASVKVSCKASGYTFTNYDINWVRQA | SEQ ID |
| Variable Domain | TGQGLEWMGWMNPHSGQTGYAQKFQGRVTMTRNTAISTA | NO: 428 |
| 10 | YMELSSLRSEDTAVYYCAREGYDLWSGHSSNWLDPWGQGT | |
| | LVTVSA | |
| Heavy Chain | QVQLVQSGAEVKKPGASVKVSCKASGYTFTNYDINWVRQA | SEQ ID |
| Variable Domain | TGQGLEWMGWMNPHSGQTGYAQKFQGRVTMTRNTAISTA | NO: 429 |
| 11 | YMELSSLRSEDTAVYYCAREGYDLWSGYSSNWLDPWGQGT | |
| | LVTVSA | |
| Heavy Chain | EVQVVESGGGLVQPGGSLRLSCAASGFTFSSYAMYWVRQSP | SEQ ID |
| Variable Domain | GKGLEWVSLITGSGGSTHYADSVKGRFTISRDNSKKTLFLQM | NO: 430 |
| 12 | NSLRAEDTAAYYCAKDEGLTTGEYWGQGTQVTVSS | |
| Heavy Chain | EVQVVESGGGLVQPGGSLRLSCAASGFTFRNYAMTWVRQSP | SEQ ID |
| Variable Domain | GKGLEWVSLISGSGGITKYADSVKGRFTISRDNSKKTLFLQM | NO: 431 |
| 13 | NSLRAEDTAAYYCVKDEGLTTGEYWGQGTQVTVSS | |
| Heavy Chain | EVQVVESGGGLVQPGGSLRLSCAASGFTFSSYAMTWVRQSP | SEQ ID |
| Variable Domain | GKGLEWVSLISGSGGITHYADSVKGRFTISRDNSKKTLFLQM | NO: 432 |
| 14 | NSLRAEDTAAYYCVKDEGLTTGEFWGQGTQVTVSS | |
| Heavy Chain | EVQVVESGGGLVQPGGSLRLSCAASGFTFSSYAMTWVRQSP | SEQ ID |
| Variable Domain | GKGLEWVSLISGSGGVTHYADSVKGRFTISRDNSKKTLFLQM | NO: 433 |
| 15 | NSLRAEDTAAYYCVKDEGLTTGEFWGQGTQVTVSS | |
| Heavy Chain | EVQVVESGGGLVQPGGSLRLSCAASGFTFSSYAMSWVRQSP | SEQ ID |
| Variable Domain | GKGLEWVSLISGSGGITHYADSVKGRFTISRDNSKKTLFLQM | NO: 434 |
| 16 | NSLRAEDTAAYYCVKDEGLTTGEYWGQGTQVTVSS | |
| Heavy Chain | EVQVVESGGGLVQPGGSLRLSCAASGFTFSSYAMSWVRQSP | SEQ ID |
| Variable Domain | GKGLEWVSLISGSGAITHYADSVKGRFTISRDNSKKTLFLQM | NO: 435 |
| 17 | NSLRAEDTAAYYCVKDEGLTTGEYWGQGTQVTVSS | |
| Heavy Chain | EVQVVESGGGLVQPGGSLRLSCAASGFTFSSYAMTWVRQSP | SEQ ID |
| Variable Domain | GKGLEWVSLISGSGGVTHYADSVKGRFTISRDNSKKTLFLQM | NO: 436 |
| 18 | NSLRAEDTAAYYCVKDEGLTTGESWGQGTQVTVSS | |
| Heavy Chain | EVQVVESGGGLVQPGGSLRLSCAASGFTFSSYAMTWVRQSP | SEQ ID |
| Variable Domain | GKGLEWVSLISGSGGITHYADSVKGRFTISRDNSKKTLFLQM | NO: 437 |
| 19 | NSLRAEDTAAYYCVKDEGLTTGESWGQGTQVTVSS | |
| Heavy Chain | EVQVVESGGGLVQPGGSLRLSCAASGFTFSSYAMTWVRQSP | SEQ ID |
| Variable Domain | GKGLEWVSLISGSGGLTKYADSVKGRFTISRDNSKKTLFLQM | NO: 438 |
| 20 | NSLRAEDTAAYYCAKEEGLTTGEYWGQGTQVTVSS | |
| Heavy Chain | EVQVVESGGGLVQPGGSLRLSCAASGFTFNTYAMTWVRQSP | SEQ ID |
| Variable Domain | GKGLEWVSLISGSGGLTKYADSVKGRFTISRDNSKKTLFLQM | NO: 439 |
| 21 | NSLRAEDTAAYYCAKEEGLTTGEYWGQGTQVTVSS | |
| Heavy Chain | EVQVVESGGGLVQPGGSLRLSCAASGFTFSSYAMTWVRQSP | SEQ ID |
| Variable Domain | GKGLEWVSLISGSGGLTNYADSVKGRFTISRDNSKKTLFLQM | NO: 440 |
| 22 | NSLRAEDTAAYYCAKDEGLTSGEYWGQGTQVTVSS | |
| Heavy Chain | EVQVVESGGGLVQPGGSLRLSCAASGFTFSSYAMTWVRQSP | SEQ ID |
| Variable Domain | GKGLEWVSLISGSGGSTHYADSVKGRFTISRDNSKKTLFLQM | NO: 441 |
| 23 | NSLRAEDTAAYYCAKDEGLTTGEYWGQGTQVTVSS | |
| Heavy Chain | EVQVVESGGGLVQPGGSLRLSCAASGFTFSSHAMTWVRQSP | SEQ ID |
| Variable Domain | GKGLEWVSLISGSGGLTKYADSVRGRFTISRDNSKKTLFLQM | NO: 442 |
| 24 | NSLRAEDTAAYYCVKEEGLTTGEYWGQGTQVTVSS | |
| Heavy Chain | EVQVVESGGGLVQPGGSLRLSCAASGFTFSSYAMTWVRQSP | SEQ ID |
| Variable Domain | GKGLEWVSLISGSGGLTKYADSVKGRFTISRDNSKKTLFLQM | NO: 443 |
| 25 | NSLRAEDTAAYYCAKDEGLTTGEYWGQGTQVTVSS | |
| Heavy Chain | EVQVVESGGGLVQPGGSLRLSCAASGFTFSSYAMTWVRQSP | SEQ ID |
| Variable Domain | GKGLEWVSLISGSGSLTKYADSVKGRFTISRDNSKKTLFLQM | NO: 444 |
| 26 | NSLRAEDTAAYYCVKEEGLTTGEYWGQGTQVTVSS | |
| Heavy Chain | EVQVVESGGGLVQPGGSLRLSCAASGFTFSSYAMTWVRQSP | SEQ ID |
| Variable Domain | GKGLEWVSLISGSGGLTKYADSVKGRFTISRDNSKKTLFLQM | NO: 445 |
| 27 | NSLRAEDTAAYYCVKDEGLTTGEYWGQGTQVTVSS | |
| Heavy Chain | EVQVVESGGGLVQPGGSLRLSCAASGFTFGNYAMTWVRQSP | SEQ ID |
| Variable Domain | GKGLEWVSLISGSGGLTKYADSVKGRFTISRDNSKKTLFLQM | NO: 446 |
| 28 | NSLRAEDTAAYYCVKDEGLTTGEYWGQGTQVTVSS | |
| Heavy Chain | EVQVVESGGGLVQPGGSLRLSCAASGFTFTNYAMTWVRQSP | SEQ ID |
| Variable Domain | GKGLEWVSLISGGGGLTYYADSVKGRFTISRDNSKKTLFLQ | NO: 447 |
| 29 | MNSLRAEDTAAYYCVKDEGLTTGEYWGQGTQVTVSS | |
| Heavy Chain | EVQVVESGGGLVQPGGSLRLSCAASGFTFSIYAMTWVRQSP | SEQ ID |
| Variable Domain | GKGLEWVSLISGSGGLTKYADSVKGRFTISRDNSKKTLFLQM | NO: 448 |
| 30 | NSLRAEDTAAYYCVKDEGLTTGEYWGQGTQVTVSS | |
| Heavy Chain | EVQVVESGGGLVQPGGSLRLSCAASGFTFSSYAMTWVRQSP | SEQ ID |
| Variable Domain | GKGLEWVSLISGSGGLTYYADSVKGRFTISRDNSKKTLFLQM | NO: 449 |
| 31 | NSLRAEDTAAYYCVKDEGLTTGEYWGQGTQVTVSS | |
| Heavy Chain | EVQVVESGGGLVQPGGSLRLSCAASGFTFSSHAMTWVRQSP | SEQ ID |
| Variable Domain | GKGLEWVSLISGSGGLTYYADSVKGRFTISRDNSKKTLFLQM | NO: 450 |
| 32 | NSLRAEDTAAYYCVKDEGLTTGEYWGQGTQVTVSS | |
| Heavy Chain | EVQVVESGGGLVQPGGSLRLSCAASGFTFSNYAMTWVRQSP | SEQ ID |
| Variable Domain | GKGLEWVSLISGGGGLTYYADSVKGRFTISRDNSKKTLFLQ | NO: 451 |
| 33 | MNSLRAEDTAAYYCVKDEGLTTGEYWGQGTQVTVSS | |
| Heavy Chain | EVQVVESGGGLVQPGGSLRLSCAASGFTFSSYAMTWVRQSP | SEQ ID |
| Variable Domain | GKGLEWVSLISGSGGLTKYADSVKGRFTISRDNSKKTLFLQM | NO: 452 |
| 34 | NSLRAEDTAAYYCVKEEGLTTGEYWGQGTQVTVSS | |
| Heavy Chain | EVQVVESGGGLVQPGGSLRLSCAASGFTFSSYAMTWVRQSP | SEQ ID |
| Variable Domain | GKGLEWVSLISGSGGITKYADSVKGRFTISRDNSKKTLFLQM | NO: 453 |
| 35 | NSLRAEDTAAYYCAKEEGLTTGEYWGQGTQVTVSS | |
| Heavy Chain | EVQLVESGGGLVQPGGSLRLSCAASGFRFSSYAMNWVRQAP | SEQ ID |
| Variable Domain | GKGLEWVSLISGSGGLTKYADSVKGRFTISRDNSRNTLYLQM | NO: 454 |
| 36 | ISLRAEDTAVYFCAKERWEPSFHYWGQGTLVTVSS | |
| Heavy Chain | EVQLVESGGGLVQPGGSLRLSCAASGFTFRSYWMHWVRQA | SEQ ID |
| Variable Domain | PGKGLVWVSRIDNNGINTRYADSVKGRFTISRDNAKNTLYL | NO: 455 |
| 37 | QMKSLRAEDTAVYYCVRDFQLELRDYWGQGTLVTVSS | |
| Heavy Chain | EVQLVESGGGLVQPGGSLRLSCAASGFTFSNYWMHWVRQA | SEQ ID |
| Variable Domain | PGKGLVWVSRISSDGISTRYADSVKGRFTISRDNAKNTLYLQ | NO: 456 |
| 38 | MKSLRAEDTAVYYCVRDFQLELPDYWGQGTLVTVSS | |
| Heavy Chain | EVQLVESGGGLVQPGGSLRLSCAASGLTFSNYWMHWVRQA | SEQ ID |
| Variable Domain | PGKGLVWVSRINSDGISTRYADSVKGRFTISRDNAKNTLYLQ | NO: 457 |
| 39 | MKSLRAEDTAVYYCVSDFQLELPDYWGQGTLVTVSS | |
| Heavy Chain | EVQLVESGGGLVQPGGSLRLSCAASGFTFSNYWMHWVRQA | SEQ ID |
| Variable Domain | PGKGLVWVSRINNDGISTRYADSVKGRFTISRDNAKNTLYLQ | NO: 458 |
| 40 | MKSLRAEDTAVYYCVRDFQLELPDYWGQGTLVTVSS | |
| Heavy Chain | EVQLVESGGGLVQPGGSLRLSCAASGFTFSSFWMHWVRQAP | SEQ ID |
| Variable Domain | GKGLVWVSRINSDGFSTRYADSVKGRFTISRDNAKNTLYLQ | NO: 459 |
| 41 | MKSLRAEDTAVYYCVRDFQLELRDFWGQGTLVTVSS | |
| Heavy Chain | EVQLVESGGGLVQPGGSLRLSCAASGFTFSSFWMHWVRQAP | SEQ ID |
| Variable Domain | GKGLVWVSRINSDGFSTRYADSVKGRFTISRDNAKNTLYLQ | NO: 460 |
| 42 | MKSLRAEDTAVYYCVRDFQLELRDYWGQGTLVTVSS | |
| Heavy Chain | EVQLVESGGGLVQPGGSLRLSCAASGFTFSSYWMHWVRQAP | SEQ ID |
| Variable Domain | GKGLVWVSRINSDGISTRYADSVKGRFTISRDNAKNTLYLQ | NO: 461 |
| 43 | MKSLRAEDTAVYYCVRDFQLELRDYWGQGTLVTVSS | |
| Heavy Chain | EVQLVESGGGFVKPGGSLRLSCAASGFTFHNAWMSWVRQA | SEQ ID |
| Variable Domain | PGKGLEWVGRIKGVTAGGTADYAAPVKGRFTISRDGSKSTL | NO: 462 |
| 44 | YLQMNSLKTEDTAVYYCTTDHPKFGFDFWGQGTLVTVSS | |
| Heavy Chain | EVQLVESGGGFVKPGGSLRLSCAASGFTFNNAWMSWVRQA | SEQ ID |
| Variable Domain | PGKGLEWVGRVKSVTAGGTTDYAAPVKGRFTISRDGSKSTL | NO: 463 |
| 45 | YLQMNSLKTEDTAVYYCTTDHPKFGFDFWGQGTLVTVSS | |
| Heavy Chain | EVQLVESGGGFVKPGGSLRLSCAASGFTFSNAWMSWVRQAP | SEQ ID |
| Variable Domain | GKGLEWVGRIKSKTDGGTTDYAAPVKGRFTISRDGSKSTLYL | NO: 464 |
| 46 | QMNSLKTEDTAVYYCTTDHPKFGFDYWGQGTLVTVSS | |
| Heavy Chain | EVQLVESGGGFVKPGGSLRLSCAASGFTFNSAWMSWVRQAP | SEQ ID |
| Variable Domain | GKGLEWVGRIKSVTTGGTTDYAAPVKGRFTISRDGSKSTLYL | NO: 465 |
| 47 | QMNSLKTEDTAVYYCTTDHPKFGFDFWGQGTLVTVSS | |
| Heavy Chain | EVQLVESGGGFVKPGGSLRLSCAASGFTFNNAWMSWVRQA | SEQ ID |
| Variable Domain | PGKGLEWVGRIKSVTAGGTTDYAAPVKGRFTISRDGSKSTLY | NO: 466 |
| 48 | LQMNSLKTEDTAVYYCTTDHPKFGFDYWGQGTLVTVSS | |
| Heavy Chain | EVQLVESGGGFVKPGGSLRLSCAASGFTFNNAWMSWVRQA | SEQ ID |
| Variable Domain | PGKGLEWVGRIKSVTAGGTTDFPAPVKGRFTISRDGSKSTLY | NO: 467 |
| 49 | LQMNSLKTEDTAVYYCTTDHPKFGFDFWGQGTLVTVSS | |
| Heavy Chain | EVQLVESGGGLVTPGGSLRISCAASGFIFNNAWMRWVRQAP | SEQ ID |
| Variable Domain | GKGLEWVGHIKSETDGGTTDYAAPVKGRFTISRDDSENTLYL | NO: 468 |
| 50 | QMSSLKTEDTAVYYCTTGGVWNYGGSLDFWGQGTLVTVSS | |
| Heavy Chain | EVQLVESGGGLVTPGGSLRISCAASGFIFNNAWMRWVRQAP | SEQ ID |
| Variable Domain | GKGLEWVGHIKSETDGGTTDYAAPVKGRFTISRDDSENTLYL | NO: 469 |
| 51 | QMSSLKTEDTAVYYCITGGVWNYGGSLDFWGQGTLVTVSS | |
| Heavy Chain | EVQLVESGGGLVTPGGSLRISCAASGFIFNNAWMRWVRQAP | SEQ ID |
| Variable Domain | GKGLEWVGHIKSETDGGTTDYAAPVKGRFTISRDDSENTLYL | NO: 470 |
| 52 | QMSSLKTEDTAVYYCTTGGVWKYGGSLDFWGQGTLVTVSS | |
| Heavy Chain | EVQLVESGGGLVTPGGSLRISCAASGFIFNNAWMRWVRQAP | SEQ ID |
| Variable Domain | GKGLEWVGHIKSETDGGTTDYAAPVKGRFTISRDDSENTLYL | NO: 471 |
| 53 | QMSSLKTEDTAVYYCATGGVWKYGGSLDFWGQGTLVTVSS | |
| Heavy Chain | EVQLVESGGGLVTPGGSLRISCAASGFTFNYAWMTWVRQAP | SEQ ID |
| Variable Domain | GKGLEWVGRIKRKTDGGTTDYAAPVQGRFTISRDDSENTLYL | NO: 472 |
| 54 | QMSSLKTEDTAVYYCTTGSVWKYGGSLDFWGQGTLVTVSS | |
| Heavy Chain | EVQLVESGGGLVTPGGSLRISCAASGFTFINAWMNWVRQAP | SEQ ID |
| Variable Domain | GKGLEWVGRIKSLTDGGTTDYAAPVKGRFTISRDDSENTLY | NO: 473 |
| 55 | LQMSSLKTEDTAVYYCSTGSVWNYGGSLDFWGQGTLVTVSS | |
| Heavy Chain | EVQLVESGGGLVTPGGSLRISCAASGFTFSNAWMNWVRQAP | SEQ ID |
| Variable Domain | GKGLEWVGRIKRKTDGGTREYAAPVKGRFTISRDDSENTLY | NO: 474 |
| 56 | LQMSSLKTEDTAVYYCTTGSVWKYGGSLDFWGQGTLVTVSS | |
| Heavy Chain | EVQLVESGGGLVTPGGSLRISCAASGFTFSNAWMSWVRQAP | SEQ ID |
| Variable Domain | GKGLEWVGRIKRNTDGGTTDYAAPVKGRFTISRDDSENTLY | NO: 475 |
| 57 | LQMSSLKTEDTAVYYCTTGSVWKYGGSLDFWGQGTLVTVSS | |
| Heavy Chain | EVQLVESGGGLVTPGGSLRISCAASGFTFSNAWMSWVRQAP | SEQ ID |
| Variable Domain | GKGLEWVGRIKTNTDGGTTDYAAPVKGRFTISRDDSENTLY | NO: 476 |
| 58 | LQMSSLKTEDTAVYYCTTGSVWNYGGSLDYWGQGTLVTVSS | |
| Heavy Chain | EVQLVESGGGLVTPGGSLRISCAASGFTFSNAWMSWVRQAP | SEQ ID |
| Variable Domain | GKGLEWVGRIKSKTYGGTTDYDAPVKGRFTISRDDSENTLY | NO: 477 |
| 59 | LQMSSLKTEDTAVYYCTTGSVWKYGGSLDYWGQGTLVTVSS | |
| Heavy Chain | EVQLVESGGGLVTPGGSLRISCAASGFTFSNAWMSWVRQAP | SEQ ID |
| Variable Domain | GKGLEWVGRIKSQTYGGTTDYAAPVKGRFTISRDDSENTLY | NO: 478 |
| 60 | LQMSSLKTEDTAVYYCTTGSVWKYGGSLDYWGQGTLVTVSS | |
| Heavy Chain | EVQLVESGGGLVTPGGSLRISCAASGFTFSNAWMSWVRQAP | SEQ ID |
| Variable Domain | GKGLEWVGRIKSKTDGGTTDYAAPVKGRFTISRDDSENTLY | NO: 479 |
| 61 | LQMSSLKTEDTAVYYCTTGSVWRYGGSLDFWGQGTLVTVSS | |
| Heavy Chain | EVQLVESGGGLVTPGGSLRISCAASGFTFSNAWMNWVRQAP | SEQ ID |
| Variable Domain | GKGLEWVGRIKSKTDGGTTDYAAPVKGRFTISRDDSENTLY | NO: 480 |
| 62 | LQMSSLKTEDTAVYYCTTGSVWNYGGSLDFWGQGTLVTVSS | |
| Heavy Chain | EVQLVESGGGLVTPGGSLRISCAASGFTFSNAWMSWVRQAP | SEQ ID |
| Variable Domain | GKGLEWVGRIKSKTDGGTTDYAAPVKGRFTISRDDSENTLY | NO: 481 |
| 63 | LQMSSLKTEDTAVYYCTTGSVWKYGGSLDFWGQGTLVTVSS | |
| Heavy Chain | EVQLVESGGGLVTPGGSLRISCAASGFTFSNAWMTWVRQAP | SEQ ID |
| Variable Domain | GKGLEWVGRIKSKTYGGTTDYAAPVKGRFTISRDDSENTLY | NO: 482 |
| 64 | LQMSSLKTEDTAVYYCTTGSVWNYGGSLDFWGQGTLVTVSS | |
| Heavy Chain | EVQLVESGGGLVTPGGSLRISCAASGFTFINAWMNWVRQAP | SEQ ID |
| Variable Domain | GKGLEWVGRIKRKTDGGTRDYAAPVKGRFTISRDDSENTLY | NO: 483 |
| 65 | LQMSSLKTEDTAVYYCTTGSVWKYGGSLDFWGQGTLVTVSS | |
| Heavy Chain | EVQLVESGGGLVKPGGSLRLSCAASGFTFSNAWMSWVRQAP | SEQ ID |
| Variable Domain | GRGLEWVGRIKRETDGGTTDYASPVKGRFTISRDHSENTLF | NO: 484 |
| 66 | LQMNSLKTEDTAVYYCTTGSSWNVYFFDFWGQGTLVTVSS | |
| Heavy Chain | EVQLVESGGGLVKPGGSLRLSCAASGFTFSTAWMSWVRQAP | SEQ ID |
| Variable Domain | GRGLEWVGRIKRKSDGGTTDYAAPVKGRFTISRDHSENTLF | NO: 485 |
| 67 | LQMNSLKTEDTAVYYCTTGSSWHVYFFDFWGQGTLVTVSS | |
| Heavy Chain | EVQLVESGGGLVKPGGSLRLSCAASGFIFSNAWMSWVRQAP | SEQ ID |
| Variable Domain | GRGLEWVGRIKSKTYGGTTDYAAPVKGRFTISRDHSENTLFL | NO: 486 |
| 68 | QMNSLKTEDTAVYYCTTGSSWSLYYFDFWGQGTLVTVSS | |
| Heavy Chain | EVQLVESGGGLVKPGGSLRLSCAASGFIFSNAWMSWVRQAP | SEQ ID |
| Variable Domain | GRGLEWVGRIKSKTDGGTTDYAAPVKGRFTISRDHSENTLFL | NO: 487 |
| 69 | QMNSLKTEDTAVYYCTTGSSWYVYYFDYWGQGTLVTVSS | |
| Heavy Chain | EVQLVESGGGLVKPGGSLRLSCAASGFIFSNAWMSWVRQAP | SEQ ID |
| Variable Domain | GRGLEWVGRIKSEIDGGTTDYAAPVKGRFTISRDHSENTLFL | NO: 488 |
| 70 | QMNSLKTEDTAVYYCTTGSSWYVYYFDFWGQGTLVTVSS | |
| Light Chain | DIQMTQSPSSLSASVGDRVTITCRASQDIRNDLAWYQQKPGK | SEQ ID |
| Variable Domain | APKRLIYASSSLQSGVPSRFSGSGSGTEFTLTISSLQPEDFATY | NO: 489 |
| 1 | YCLQHNNYPFTFGPGTKVDIK | |
| Light Chain | DIQMTQSPSSLSASVGDSVIITCQASQGITNYLNWYQQKPGK | SEQ ID |
| Variable Domain | APNLLIYDASNLETGVPSRFSGSGSGTYFTFTISSLQPEDIATY | NO: 490 |
| 2 | FCQQYENLPYTFGQGTKLEIK | |
| Light Chain | DIQMTQSPSSLSASVGDRVTITCRASQGIRNDLGWYQQKPGK | SEQ ID |
| Variable Domain | APKRLIYAASSLQSGVPSRFSGSGSGTEFTLTISSLQPEDFATY | NO: 491 |
| 3 | YCLQHNSYPWSFGQGTKVEIK | |
| Light Chain | DIQMTQSPSSLSASVGDRVTITCRASQGIRDDLAWYQQKAGK | SEQ ID |
| Variable Domain | APKRLIFAASSLQSGVPSRFSGSGSGTEFTLTISGLQPEDFANY | NO: 492 |
| 4 | YCLQHKSNPFTFGQGTKLEIK | |
| Light Chain | DIVMTQSPDSLAVSLGERATIDCKSSQSVFYSSNNKNYLAWF | SEQ ID |
| Variable Domain | QQKPGQPPKLLIYWASTRESGVPDRFSGSGSGTDFTLTISSLQ | NO: 493 |
| 5 | AEDVAVYYCQQHYDIPRTFGQGTKVEIK | |
| Light Chain | DIQMTQSPSSLSASVGDRVTFTCRASQGISRFLNWYQQKPGK | SEQ ID |
| Variable Domain | APKLLIYAASTLQNGVPSRFSGSGSGTDFTLTISSLQPEDFATY | NO: 494 |
| 6 | FCQQTYSTPLTFGGGTKVEIY | |
| Light Chain | DIQMTQSPSSLSASVGDSVTITCRASQSISNYLNWYHQKPGK | SEQ ID |
| Variable Domain | APKLLIYAAYRLQTGVPSRFSGSGSGTDFTLTISSLQPEDFAT | NO: 495 |
| 7 | YYCQQSYSSPLTFGGGTKVEIK | |
| Light Chain | DIQLTQSPSFLSASVGDRVTITCRASQGISSYLAWYQQKPGKA | SEQ ID |
| Variable Domain | PKLLIYAASTLQSGFRSRFSGSGSGTEFTLTISSLQPEDFASYY | NO: 496 |
| 8 | CQQLNSYPRTFGQGTKVEIK | |
| Light Chain | DIQLTQSPSFLSASVGDRVTITCRASQGISSYLAWYQQKPGKA | SEQ ID |
| Variable Domain | PKLLIYAASTLQSGFRSRFSGSGSGTEFTLTISSLQPEDFASYY | NO: 497 |
| 9 | CQQLNNYPRTFGQGTKVEIK | |
| Light Chain | DIQLTQSPSFLSASVGDRVTITCRASQGISSYLAWYQQKPGKA | SEQ ID |
| Variable Domain | PKLLIYAASTLQSGFRSRFSGSGSGTEFTLTISSLQPEDFASYY | NO: 498 |
| 10 | CQQLQSYPRTFGQGTKVEIK | |
| Light Chain | DIQMTQSPSSLSASVGDRVTITCRASQGIRNDLGWYQQKPGK | SEQ ID |
| Variable Domain | APKRLIYVVSSFQSGVPSRFSGSGSGTEFTLTISSLQPEDFATY | NO: 499 |
| 11 | YCLQHNNYPFTFGPGTKVDIK | |
| Light Chain | DIQMTQSPSSLSASVGDRVTITCRASQDIRNDLDWYQQKPGK | SEQ ID |
| Variable Domain | APKRLIYATSSLQSGVPSRFSGSGSGTEFTLTISSLQPEDFATY | NO: 500 |
| 12 | YCLQHNSYPFTFGPGTKVDIK | |
| Light Chain | DIQMTQSPSSLSASVGDRVTITCRASQAIRNDLGWYQQKPGK | SEQ ID |
| Variable Domain | APKRLIYATSNLQSGVPSRFSGSGSGTEFTLTISSLQPEDFATY | NO: 501 |
| 13 | YCLQHNSYPFTFGPGTKVDIK | |
| Light Chain | DIQMTQSPSSLSASVGDRVTITCRASQGIRNDLGWYQQKPGK | SEQ ID |
| Variable Domain | APKRLIYATSSLQSGVPSRFSGSGSGTEFTLTISSLQPEDFATY | NO: 502 |
| 14 | YCLQHNSYPFTFGPGTKVDIK | |
| Light Chain | DIQMTQSPSSLSASVGDRVTITCRTSQGIRNDLAWYQQKPGK | SEQ ID |
| Variable Domain | APKRLIYAASSLQSGVPSRFSGSGSGTEFTLTISSLQPEDFATY | NO: 503 |
| 15 | YCLQHNNYPFTFGPGTKVDIK | |
| Light Chain | DIQMTQSPSSLSASVGDRVTITCRASQGIRNDLAWYQQKPGK | SEQ ID |
| Variable Domain | APKRLIYAASSFQSGVPSRFSGSGSGTEFTLTISSLQPEDFATY | NO: 504 |
| 16 | YCLQHNSYPFTFGPGTKVDIK | |
| Light Chain | DIQMTQSPSSLSASVGDRVTITCRASQGIRNDLGWYQQKPGK | SEQ ID |
| Variable Domain | APKRLIYAASSLKSGVPSRFSGSGSGTEFTLTISSLQPEDFATY | NO: 505 |
| 17 | YCLQHNSYPFTFGPGTKVDIK | |
| Light Chain | DIQMTQSPSSLSASVGDRVTITCRASQGIRNDLGWYQQKPGK | SEQ ID |
| Variable Domain | APKRLIYAASSLQSGVPSRFSGSGSGTEFTLTISSLQPEDFATY | NO: 506 |
| 18 | YCLQHNSYPFTFGPGTKVDIK | |
| Light Chain | DIQMTQSPSSLSASVGDRVTITCRASQGIRNDLGWYQQKPGK | SEQ ID |
| Variable Domain | APKRLIYATSSFQSGVPSRFSGSGSGTEFTLTISSLQPEDFATY | NO: 507 |
| 19 | YCLQHNSYPFTFGPGTKVDIK | |
| Light Chain | DIQMTQSPSSLSASVGDRVTITCRASQGIRNDLGWYQQKPGK | SEQ ID |
| Variable Domain | APKRLIYAASSFQSGVPSRFSGSGSGTEFTLTISSLQPEDFATY | NO: 508 |
| 20 | YCLQHNSYPFTFGPGTKVDIK | |
| Light Chain | DIQMTQSPSSLSASVGDRVTITCRASQGIRNNLGWYQQKPGK | SEQ ID |
| Variable Domain | APKRLIYAASSLQSGVPSRFSGSGSGTEFTLTISSLQPEDFATY | NO: 509 |
| 21 | YCLQHNSYPFTFGPGTKVDIK | |
| Light Chain | DIQMTQSPSSLSASVGDRVTITCRASQGIRNDLGWYQQKPGK | SEQ ID |
| Variable Domain | APKRLIYAASSLQSGVPSRFSGSGSGTEFTLTISSLQPEDFATY | NO: 510 |
| 22 | YCLQHNTYPFSFGPGTKVDIK | |
| Light Chain | DIQMTQSPSSLSASVGDRVTITCRASQGIRNDLGWYQQKPGK | SEQ ID |
| Variable Domain | APKRLIYAASSLQSGVPSRFSGSGSGTEFTLTISSLQPEDFATY | NO: 511 |
| 23 | YCLQHSSYPFTFGPGTKVDIK | |
| Light Chain | DIQMTQSPSSLSASVGDRVTITCRASQGIRNDLGWYQQKPGK | SEQ ID |
| Variable Domain | APKRLIYAASSLQSGVPSRFSGSGSGTEFTLTISSLQPEDFATY | NO: 512 |
| 24 | YCLQHNNYPFTFGPGTKVDIK | |
| Light Chain | DIQMTQSPSSLSASVGDRVTITCRASQGIRNDLGWYQQKPGK | SEQ ID |
| Variable Domain | APKRLIYAVSSLQSGVPSRFSGSGSGTEFTLTISSLQPEDFATY | NO: 513 |
| 25 | YCLQHNSYPFTFGPGTKVDIK | |
| Light Chain | DIQMTQSPSSLSASVGDRVTITCRASQGIRNDLGWYQQKPGK | SEQ ID |
| Variable Domain | APKRLIYAASSLQSGVPSRFSGSGSGTEFTLTISSLQPEDFATY | NO: 514 |
| 26 | YCLQHNTYPFTFGPGTKVDIK | |
| Light Chain | DIQMTQSPSSLSASVGDRVTITCRASQGIRNDLGWYQQKPGK | SEQ ID |
| Variable Domain | APKRLIYASSSLQSGVPSRFSGSGSGTEFTLTISSLQPEDFATY | NO: 515 |
| 27 | YCLQHNNYPFTFGPGTKVDIK | |
| Light Chain | DIQMTQSPSSLSASVGDRVTITCRASQGIRNDLAWYQQKAGK | SEQ ID |
| Variable Domain | APKRLIFATSILQSGVPSRFSGSGSGTEFTLTISGLQPEDFANY | NO: 516 |
| 28 | YCLQHNSNPFTFGQGTKLEIK | |
| Light Chain | DIQMTQSPSSLSASVGDRVTITCRASQGIRNDLGWYQQKAGK | SEQ ID |
| Variable Domain | APKRLIFAASSLQSGVPSRFSGSGSGTEFTLTISGLQPEDFANY | NO: 517 |
| 29 | YCLQHKSNPFTFGQGTKLEIK | |
| Light Chain | DIQMTQSPSSLSASVGDRVTITCRASQAIRDDLAWYQQKAGK | SEQ ID |
| Variable Domain | APKRLIFAASSLQNGVPSRFSGSGSGTEFTLTISGLQPEDFANY | NO: 518 |
| 30 | YCLQHKSNPFTFGQGTKLEIK | |
| Light Chain | DIQMTQSPSSLSASVGDRVTITCRASQGIRNDLAWYQQKAGK | SEQ ID |
| Variable Domain | APKRLIFAASILQSGVPSRFSGSGSGTEFTLTISGLQPEDFANY | NO: 519 |
| 31 | YCLQHNSNPFTFGQGTKLEIK | |
| Light Chain | DIQMTQSPSSLSASVGDRVTITCRASQGIRNDLAWYQQKAGK | SEQ ID |
| Variable Domain | APKRLIFAASILQSGVPSRFSGSGSGTEFTLTISGLQPEDFANY | NO: 520 |
| 32 | YCLQHKSNPFTFGQGTKLEIK | |
| Light Chain | DIQMTQSPSSLSASVGDRVTITCRASQGIRNDLAWYQQKAGK | SEQ ID |
| Variable Domain | APKRLIFAASSLQSGVPSRFSGSGSGTEFTLTISGLQPEDFANY | NO: 521 |
| 33 | YCLQHNSNPFTFGQGTKLEIK | |
| Light Chain | DIQMTQSPSSLSASVGDRVTITCRASQGIRNDLAWYQQKAGK | SEQ ID |
| Variable Domain | APKRLIFAASILQSGVPSRFSGSGSGTEFTLTISGLQPEDFANY | NO: 522 |
| 34 | YCLQHYSNPFTFGQGTKLEIK | |
| Light Chain | DIVMTQSPDSLAVSLGERATIDCKSSQSVFYTSNSKHYLNWF | SEQ ID |
| Variable Domain | QQKPGQPPKLLIYWASTRESGVPDRFSGSGSGTDFTLTISSLQ | NO: 523 |
| 35 | AEDVAVYYCQQYYSIPRTFGQGTKVEIK | |
| Light Chain | DIVMTQSPDSLAVSLGERATIDCKSSQSVLYSSNNKNYLAWF | SEQ ID |
| Variable Domain | QQKPGQPPKLLIYWASTRESGVPDRFSGSGSGTDFTLTISSLQ | NO: 524 |
| 36 | AEDVAVYYCQQHYSTPRTFGQGTKVEIK | |
| Light Chain | DIVMTQSPDSLAVSLGERATIDCKSSQSVFYTSNSKHYLNWF | SEQ ID |
| Variable Domain | QQKPGQPPKLLIYWASTRESGVPDRFSGSGSGTDFTLTISSLQ | NO: 525 |
| 37 | AEDVAVYYCQQYYSTPRTFGQGTKVEIK | |
| Light Chain | DIVMTQSPDSLAVSLGERATIDCKSSQSVFYTSNNKNYLNWF | SEQ ID |
| Variable Domain | QQKPGQPPKLLIYWASTRESGVPDRFSGSGSGTDFTLTISSLQ | NO: 526 |
| 38 | AEDVAVYYCQQYYSTPRTFGQGTKVEIK | |
| Light Chain | DIVMTQSPDSLAVSLGERATIDCKSSQSVFYTSSNKHYLNWF | SEQ ID |
| Variable Domain | QQKPGQPPKLLIYWASTRESGVPDRFSGSGSGTDFTLTISSLQ | NO: 527 |
| 39 | AEDVAVYYCQQYYSTPRTFGQGTKVEIK | |
| Light Chain | DIQMTQSPSSLSASVGDRVTFTCRASQSITRYLNWYQQKPGK | SEQ ID |
| Variable Domain | APKLLIYAASSLQSGVPSRFSGSGSGTDFTLTISSLQPEDFATY | NO: 528 |
| 40 | FCQQSYSTPLTFGGGTKVEIY | |
| Light Chain | DIQMTQSPSSLSASVGDRVTFTCRASQFITRYLNWYQQKPGK | SEQ ID |
| Variable Domain | APKLLIYAASSLQSGVPSRFSGSGSGTDFTLTISSLQPEDFATY | NO: 529 |
| 41 | FCQQSYSTPLTFGGGTKVEIY | |
| Light Chain | DIQMTQSPSSLSASVGDRVTFTCRASQSITRYLNWYQQKPGK | SEQ ID |
| Variable Domain | APKLLIYAASSLQSGVPSRFSGSGSGTDFTLTISSLQPEDFATY | NO: 530 |
| 42 | FCQQSYNTPLTFGGGTKVEIY | |
| Light Chain | DIQMTQSPSSLSASVGDRVTFTCRASQGISRYLNWYQQKPGK | SEQ ID |
| Variable Domain | APKLLIYGASTLQSGVPSRFSGSGSGTDFTLTISSLQPEDFATY | NO: 531 |
| 43 | FCQQSYSTPLTFGGGTKVEIY | |
| Light Chain | DIQMTQSPSSLSASVGDRVTFTCRASQSITRSLNWYQQKPGK | SEQ ID |
| Variable Domain | APKLLIYAASRLQSGVPSRFSGSGSGTDFTLTISSLQPEDFATY | NO: 532 |
| 44 | FCQQSYSSPLTFGGGTKVEIY | |
| Light Chain | DIQMTQSPSSLSASVGDRVTFTCRASQSISRYLNWYQQKPGK | SEQ ID |
| Variable Domain | APKLLIYAASSLQSGVPSRFSGSGSGTDFTLTISSLQPEDFATY | NO: 533 |
| 45 | FCQQSYSTPVTFGGGTKVEIY | |
| Light Chain | DIQMTQSPSSLSASVGDRVTFTCRASQSISRYLNWYQQKPGK | SEQ ID |
| Variable Domain | APKLLIYAASSLQNGVPSRFSGSGSGTDFTLTISSLQPEDFATY | NO: 534 |
| 46 | FCQQSYSTPVTFGGGTKVEIY | |
| Light Chain | DIQMTQSPSSLSASVGDRVTFTCRASQSISRYLNWYQQKPGK | SEQ ID |
| Variable Domain | APKLLIYGASSLESGVPSRFSGSGSGTDFTLTISSLQPEDFATY | NO: 535 |
| 47 | FCQQSYRTSLTFGGGTKVEIY | |
| Light Chain | DIQMTQSPSSLSASVGDRVTFTCRASQNISRYLNWYQQKPGK | SEQ ID |
| Variable Domain | APKLLIYAASSLQSGVPSRFSGSGSGTDFTLTISSLQPEDFATY | NO: 536 |
| 48 | FCQQSYSTPLTFGGGTKVEIY | |
| Light Chain | DIQMTQSPSSLSASVGDRVTFTCRASQIISRYLNWYQQKPGK | SEQ ID |
| Variable Domain | APKLLIYAASSLQSGVPSRFSGSGSGTDFTLTISSLQPEDFATY | NO: 537 |
| 49 | FCQQSYGTPLTFGGGTKVEIY | |
| Light Chain | DIQMTQSPSSLSASVGDRVTFTCRASQSISRYLNWYQQKPGK | SEQ ID |
| Variable Domain | APKLLIYGASSLQSGVPSRFSGSGSGTDFTLTISSLQPEDFATY | NO: 538 |
| 50 | FCQQSYSTPLTFGGGTKVEIY | |
| Light Chain | DIQMTQSPSSLSASVGDRVTFTCRASQSISRYLNWYQQKPGK | SEQ ID |
| Variable Domain | APKLLIYAASSLQSGVPSRFSGSGSGTDFTLTISSLQPEDFATY | NO: 539 |
| 51 | FCQQSYSTPLTFGGGTKVEIY | |
| Light Chain | DIQMTQSPSSLSASVGDSVTITCRASQSMSSHLNWYHQKPGK | SEQ ID |
| Variable Domain | APKLLIYAASRLESGVPSRFSGSGSGTDFTLTISSLQPEDFATY | NO: 540 |
| 52 | YCQQSYSSPLTFGGGTKVEIK | |
| Light Chain | DIQMTQSPSSLSASVGDSVTITCRASQSISRYLNWYHQKPGK | SEQ ID |
| Variable Domain | APKLLIYAASSLQSGVPSRFSGSGSGTDFTLTISSLQPEDFATY | NO: 541 |
| 53 | YCQQSYSSPLTFGGGTKVEIK | |
| Light Chain | DIQMTQSPSSLSASVGDSVTITCRASQSISTYLNWYHQKPGK | SEQ ID |
| Variable Domain | APKLLIYAASSLQSGVPSRFSGSGSGTDFTLTISSLQPEDFATY | NO: 542 |
| 54 | YCQQSYSSPLTFGGGTKVEIK | |
| Light Chain | DIQMTQSPSSLSASVGDSVTITCRASQSISSYLNWYHQKPGKA | SEQ ID |
| Variable Domain | PKLLIYAASNLQSGVPSRFSGSGSGTDFTLTISSLQPEDFATYY | NO: 543 |
| 55 | CQQSYSTPFTFGGGTKVEIK | |
| Light Chain | DIQMTQSPSSLSASVGDSVTITCRASQSISSYLNWYHQKPGKA | SEQ ID |
| Variable Domain | PKLLIYAASSLQSGVPSRFSGSGSGTDFTLTISSLQPEDFATYY | NO: 544 |
| 56 | CQQSYRSPFTFGGGTKVEIK | |
| Consensus | EGYDLWSGX₁SSNWLDP, where X₁ = D, E, H, or Y | SEQ ID |
| CDRH3 (Kabat) | | NO: 545 |
| Consensus | GYDLWSGX₁SSNWLD, where X₁ = D, E, H, or Y | SEQ ID |
| CDRH3 | | NO: 546 |
| (Chothia) | | |
| Consensus | AREGYDLWSGX₁SSNWLDP, where X₁ = D, E, H, or Y | SEQ ID |
| CDRH3 (IMGT) | | NO: 547 |
| Consensus | QQLX₁X₂YPRT, where X₁ = N or Q, and X₂ = S or N | SEQ ID |
| CDRL3 (Kabat) | | NO: 548 |
| Consensus | LX₁X₂YPR, where X₁ = N or Q, and X₂ = S or N | SEQ ID |
| CDRL3 | | NO: 549 |
| (Chothia) | | |
| Consensus | QQLX₁X₂YPRT, where X₁ = N or Q, and X₂ = S or N | SEQ ID |
| CDRL3 (IMGT) | | NO: 550 |
| Consensus | X₁X₂AMX₃, where X₁ = N, S, T, or I, X₂ = Y or H, | |
| CDRH1 (Kabat) | and X₃ = T, Y, or S | |
| Consensus | LIX₁GX₂GX₃X₄TX₅YADSVX₆G, where X₁ = S or T, | SEQ ID |
| CDRH2 (Kabat) | X₂ = S or G, X₃ = G, A, or S, | NO: 552 |
| | X₄ = L, S, I, or V, X₅ = K, H, N, or Y, and | |
| | X₆ = K or R | |
| Consensus | X₁EGLTX₂GEX₃, where X₁ = D or E, X₂ = T or S, | SEQ ID |
| CDRH3 (Kabat) | and X₃ = Y, F, or S | NO: 553 |
| Consensus | RX₁SQX₂IRNX₃LX₄, where X₁ = A or T, | SEQ ID |
| CDRL1 (Kabat) | X₂ = D, G, or A, X₃ = D or N, and | NO: 554 |
| | X₄ = A, G, or D | |
| Consensus | X₁X₂SX₃X₄X₅S, where X₁ = A or V, | |
| CDRL2 (Kabat) | X₂ = S, V, T, or A, X₃ = S or N, X₄ = L or F, | |
| | and X₅ = Q or K | |
| Consensus | LQHX₁X₂YPFX₃, where X₁ = N or S, X₂ = N, S, or | SEQ ID |
| CDRL3 (Kabat) | T, and X₃ = T or S | NO: 556 |
| Consensus | GFTFX₁X₂X₃, where X₁ = S, R, N, G, or T, | SEQ ID |
| CDRH1 | X₂ = N, S, T, or I, and X₃ = Y or H | NO: 557 |
| (Chothia) | | |
| Consensus | X1GX2GX3X4, where X1 = S or T, X2 = S or G, | |
| CDRH2 | X3 = G, A, or S, and X4 = L, S, I, or V | |
| (Chothia) | | |
| Consensus | EGLTX₁GE, where X₁ = T or S | SEQ ID |
| CDRH3 | | NO: 559 |
| (Chothia) | | |
| Consensus | SQX₁IRNX₂, where X₁ = D, G, or A, and | SEQ ID |
| CDRL1 | X₂ = D or N | NO: 560 |
| (Chothia) | | |
| Consensus | X₁X₂S, where X₁ = A or V, and | |
| CDRL2 | X₂ = S, V, T, or A | |
| (Chothia) | | |
| Consensus | HX₁X₂YPF, where X₁ = N or S, and | SEQ ID |
| CDRL3 | X₂ = N, S, or T | NO: 562 |
| (Chothia) | | |
| Consensus | GFTFX₁X₂X₃A, where X₁ = S, R, N, G, or T, | SEQ ID |
| CDRH1 (IMGT) | X₂ = N, S, T, or I, and X₃ = Y or H | NO: 563 |
| Consensus | IX₁GX₂GX₃X₄T, where X₁ = S or T, X₂ = S or G, | SEQ ID |
| CDRH2 (IMGT) | X₃ = G, A, or S, and X₄ = L, S, I, or V | NO: 564 |
| Consensus | X₁KX₂EGLTX₃GEX₄, where X₁ = V or A, X₂ = D or E, | SEQ ID |
| CDRH3 (IMGT) | X₃ = T or S, and X₄ = Y, F, or S | NO: 565 |
| Consensus | QX₁IRNX₂, where X₁ = D, G, or A, and | SEQ ID |
| CDRL1 (IMGT) | X₂ = D or N | NO: 566 |
| Consensus | X₁X₂S, where X₁ = A or V, and | |
| CDRL2 (IMGT) | X₂ = S, V, T, or A | |
| Consensus | LQHX₁X₂YPFX₃, where X₁ = N or S, X₂ = N, S, or | SEQ ID |
| CDRL3 (IMGT) | T, and X₃ = T or S | NO: 568 |
| Consensus | X₁X₂WMH, where X₁ = N or S, and X₂ = Y or F | |
| Variants of 122 | | |
| CDRH1 (Kabat) | | |
| Consensus | RIX₁X₂X₃GX₄X₅TRYADSVKG, where X₁ = N, D, or S, | SEQ ID |
| CDRH2 (Kabat) | X₂ = S or N, X₃ = D or N, X₄ = I or F, and | NO: 570 |
| | X₅ = S or N | |
| Consensus | DFQLELX₁DX₂, where X₁ = P or R, and X₂ = Y or F | SEQ ID |
| CDRH3 (Kabat) | | NO: 571 |
| Consensus | RASQX₁IRX₂DLX₃, where X₁ = G or A, X₂ = D or N, | SEQ ID |
| CDRL1 (Kabat) | and X₃ = A or G | NO: 572 |
| Consensus | AX₁SX₂LQX₃, where X₁ = A or T, X₂ = S or I, and | SEQ ID |
| CDRL2 (Kabat) | X₃ = S or N | NO: 573 |
| Consensus | LQHX₁SNPFT, where X₁ = K, N, or Y | SEQ ID |
| CDRL3 (Kabat) | | NO: 574 |
| Consensus | GX₁TFX₂X₃X₄, where X₁ = F or L, X₂ = S or R, and | |
| CDRH1 | X₃ = N or S, and X₄ = Y or F | |
| (Chothia) | | |
| Consensus | X₁X₂X₃GX₄X₅, where X₁ = N, D, or S, X₂ = S or N, | |
| CDRH2 | X₃ = D or N, X₄ = I or F, and X₅ = S or N | |
| (Chothia) | | |
| Consensus | FQLELX₁D, where X₁ = P or R | SEQ ID |
| CDRH3 | | NO: 577 |
| (Chothia) | | |
| Consensus | SQX₁IRX₂D, where X₁ = G or A, and X₂ = D or N | SEQ ID |
| CDRL1 | | NO: 578 |
| (Chothia) | | |
| Consensus | AX₁S, where X₁ = A or T | |
| CDRL2 | | |
| (Chothia) | | |
| Consensus | HX₁SNPF, where X₁ = K, N, or Y | SEQ ID |
| CDRL3 | | NO: 580 |
| (Chothia) | | |
| Consensus | GX₁TFX₂X₃X₄W, where X₁ = F or L, X₂ = S or R, | SEQ ID |
| CDRH1 (IMGT) | and X₃ = N or S, and X₄ = Y or F | NO: 581 |
| Consensus | IX₁X₂X₃GX₄X₅T, where X₁ = N, D, or S, | |
| CDRH2 (IMGT) | X₂ = S or N, X₃ = D or N, X₄ = I or F, and | |
| | X₅ = S or N | |
| Consensus | VX₁DFQLELX₂DX₃, where X₁ = R or S, X₂ = P or R, | SEQ ID |
| CDRH3 (IMGT) | and X₃ = Y or F | NO: 583 |
| Consensus | QX₁IRX₂D, where X₁ = G or A, X₂ = D or N | SEQ ID |
| CDRL1 (IMGT) | | NO: 584 |
| Consensus | AX₁S, where X₁ = A or T | |
| CDRL2 (IMGT) | | |
| Consensus | LQHX₁SNPFT, where X₁ = K, N, or Y | SEQ ID |
| CDRL3 (IMGT) | | NO: 586 |
| Variants of 127 | X₁AWMS, where X₁ = N or S | SEQ ID |
| Consensus | | NO: 587 |
| CDRH1 (Kabat) | | |
| Consensus | RX₁KX₂X₃X₄X₅GGTX₆DX₇X₈APX₉X₁₀G, where X₁ = I or V, | SEQ ID |
| CDRH2 (Kabat) | X₂ = S or G, X₃ = N, V, or K, X₄ = S or T, | NO: 588 |
| | X₅ = Y, A, D, or T, X₆ = T or A, X₇ = Y or F, | |
| | X₈ = A or P, X₉ = A or V, and X₁₀ = I or K | |
| Consensus | DHPKFGFDX₁, where X₁ = F or Y | SEQ ID |
| CDRH3 (Kabat) | | NO: 589 |
| Consensus | KSSQSVX₁YX₂SX₃X₄KXYLX₆, where X₁ = F or L, | SEQ ID |
| CDRL1 (Kabat) | X₂ = S or T, and X₃ = N or S, X₄ = N or S, | NO: 590 |
| | X₅ = N or H, and X₆ = A or N | |
| Consensus | QQX₁YX₂X₃PRT, where X₁ = H or Y, X₂ = D or S, | SEQ ID |
| CDRL3 (Kabat) | and X₃ = I or T | NO: 591 |
| Consensus | GFTFX₁X₂A, where X₁ = S, H, or N, X₂ = N or S | SEQ ID |
| CDRH1 | | NO: 592 |
| (Chothia) | | |
| Consensus | KX₁X₂X₃X₄GGT, where X₁ = S or G, | SEQ ID |
| CDRH2 | X₂ = N, V, or K, X₃ = S or T, and | NO: 593 |
| (Chothia) | X₄ = Y, A, D, or T | |
| Consensus | SQSVX₁YX₂SX₃X₄KX₅Y, where X₁ = F or L, | SEQ ID |
| CDRL1 | X₂ = S or T, and X₃ = N or S, X₄ = N or S, and | NO: 594 |
| (Chothia) | X₅ = N or H | |
| Consensus | X₁YX₂X₃PR, where X₁ = H or Y, X₂ = D or S, and | |
| CDRL3 | X₃ = I or T | |
| (Chothia) | | |
| Consensus | GFTFX₁X₂AW, where X₁ = S, H, or N, X₂ = N or S | SEQ ID |
| CDRH1 (IMGT) | | NO: 596 |
| Consensus | X₁KX₂X₃X₄X₅GGTX₆, where X₁ = I or V, X₂ = S or G, | SEQ ID |
| CDRH2 (IMGT) | X₃ = N, V, or K, X₄ = S or T, | NO: 597 |
| | X₅ = Y, A, D, or T, X₆ = T or A | |
| Consensus | TTDHPKFGFDX₁, where X₁ = F or Y | SEQ ID |
| CDRH3 (IMGT) | | NO: 598 |
| Consensus | QSVX₁YX₂SX₃X₄KX₅Y, where X₁ = F or L, | SEQ ID |
| CDRL1 (IMGT) | X₂ = S or T, and X₃ = N or S, X₄ = N or S, | NO: 599 |
| | X₅ = N or H | |
| Consensus | QQX₁YX₂X₃PRT, where X₁ = H or Y, X₂ = D or S, | SEQ ID |
| CDRL3 (IMGT | and X₃ = I or T | NO: 600 |
| Variants of 128 | X₁AWMX₂, where X₁ = N or Y, and | |
| Consensus | X₂ = T, R, N, or S | |
| CDRH1 (Kabat) | | |
| Consensus | X₁IKX₂X₃X₄X₅GGTX₆X₇X₈X₉APVX₁₀G, where X₁ = R or H, | SEQ ID |
| CDRH2 (Kabat) | X₂ = S, R, or T, X₃ = I, E, K, L, N, or Q, | NO: 602 |
| | X₄ = S or T, X₅ = Y or D, X₆ = T or R, | |
| | X₇ = D or E, X₈ = S or Y, X₉ = A or D, and | |
| | X₁₀ = K or Q | |
| Consensus | GX₁VWX₂YGGSLDX₃, where X₁ = S or G, | SEQ ID |
| CDRH3 (Kabat) | X₂ = K, N, or R, and X₃ = F or Y | NO: 603 |
| Consensus | RASQX₁IX₂RX₃LN, where X₁ = G, S, F, N, or I, | SEQ ID |
| CDRL1 (Kabat) | X₂ = S or T, and X₃ = F, Y, or S | NO: 604 |
| Consensus | X₁ASX₂LX₃X₄, where X₁ = A or G, X₂ = T, S, or R, | |
| CDRL2 (Kabat) | X₃ = Q or E, and X₄ = N or S | |
| Consensus | QQX₁YX₂X₃X₄X₅T, where X₁ = T or S, | SEQ ID |
| CDRL3 (Kabat) | X₂ = S, N, R, or G, X₃ = T or S, X₄ = P or S, | NO: 606 |
| | and X₅ = L or V | |
| Consensus | GFX₁FX₂X₃A, where X₁ = T or I, X₂ = N, I, or S, | SEQ ID |
| CDRH1 | and X₃ = N, or Y | NO: 607 |
| (Chothia) | | |
| Consensus | KX₁X₂X₃X₄GGT, where X₁ = S, R, or T, | SEQ ID |
| CDRH2 | X₂ = I, E, K, L, N, or Q, X₃ = S or T, and | NO: 608 |
| (Chothia) | X₄ = Y or D | |
| Consensus | X₁VWX₂YGGSLD, where X₁ = S or G, and | SEQ ID |
| CDRH3 | X₂ = K, N, or R | NO: 609 |
| (Chothia) | | |
| Consensus | SQX₁IX₂RX₃, where X₁ = G, S, F, N, or I, | SEQ ID |
| CDRL1 | X₂ = S or T, and X₃ = F, Y, or S | NO: 610 |
| (Chothia) | | |
| Consensus | X₁AS, where X₁ = A or G | |
| CDRL2 | | |
| (Chothia) | | |
| Consensus | X₁YX₂X₃X₄X₅, where X₁ = T or S, | |
| CDRL3 | X₂ = S, N, R, or G, X₃ = T or S, X₄ = P or S, | |
| (Chothia) | and X₅ = L or V | |
| Consensus | GFX₁FX₂X₃AW, where X₁ = T or I, X₂ = N, I, or S, | SEQ ID |
| CDRH1 (IMGT) | and X₃ = N, or Y | NO: 613 |
| Consensus | IKX₁X₂X₃X₄GGTX₅, where X₁ = S, R, or T, | SEQ ID |
| CDRH2 (IMGT) | X₂ = I, E, K, L, N, or Q, X₃ = S or T, | NO: 614 |
| | X₄ = Y or D, and X₅ = T or R | |
| Consensus | X₁TGX₂VWX₃YGGSLDX₄, where X₁ = S, T, I, or A, | SEQ ID |
| CDRH3 (IMGT) | X₂ = S or G, X₃ = K, N, or R, and X₄ = F or Y | NO: 615 |
| Consensus | QX₁IX₂RX₃, where X₁ = G, S, F, N, or I, | |
| CDRL1 (IMGT) | X₂ = S or T, and X₃ = F, Y, or S | |
| Consensus | X₁AS, where X₁ = A or G | |
| CDRL2 (IMGT) | | |
| Consensus | QQX₁YX₂X₃X₄X₅T, where X₁ = T or S, | SEQ ID |
| CDRL3 (IMGT) | X₂ = S, N, R, or G, X₃ = T or S, X₄ = P or S, | NO: 618 |
| | and X₅ = L or V | |
| Variants of 130 | X₁AWMS, where X₁ = Y, N, or T | SEQ ID |
| Consensus | | NO: 619 |
| CDRH1 (Kabat) | | |
| Consensus | RIKX₁X₂X₃X₄GGTTDYAX₅PVKG, where X₁ = S or R, | SEQ ID |
| CDRH2 (Kabat) | X₂ = E or K, X₃ = T, S, or I, X₄ = D or Y, | NO: 620 |
| | and X₅ = A or S | |
| Consensus | GSSWX₁X₂YX₃FDX₄, where X₁ = N, H, S or Y, | SEQ ID |
| CDRH3 (Kabat) | X₂ = V or L, X₃ = F or Y, and X₄ = F or Y | NO: 621 |
| Consensus | RASQSX₁SX₂X₃LN, where X₁ = I or M, | SEQ ID |
| CDRL1 (Kabat) | X₂ = N, S, R or T, and X₃ = Y or H | NO: 622 |
| Consensus | AAX₁X₂LX₃X₄, where X₁ = Y or S, X₂ = R, S, or N, | |
| CDRL2 (Kabat) | X₃ = Q or E, and X₄ = T or S | |
| Consensus | QQSYX₁X₂PX₃T, where X₁ = S or R, X₂ = S or T, | SEQ ID |
| CDRL3 (Kabat) | and X₃ = L or F | NO: 624 |
| Consensus | GFX₁FSX₂A, where X₁ = T or I, and | SEQ ID |
| CDRH1 | X₂ = Y, N, or T | NO: 625 |
| (Chothia) | | |
| Consensus | KX₁X₂X₃X₄GGT, where X₁ = S or R, X₂ = E or K, | SEQ ID |
| CDRH2 | X₃ = T, S, or I, X₄ = D or Y | NO: 626 |
| (Chothia) | | |
| Consensus | SSWX₁X₂YX₃FD, X₁ = N, H, S or Y, X₂ = V or L, | SEQ ID |
| CDRH3 | and X₃ = F or Y | NO: 627 |
| (Chothia) | | |
| Consensus | SQSX₁SX₂X₃, where X₁ = I or M, | SEQ ID |
| CDRL1 | X₂ = N, S, R or T, and X₃ = Y or H | NO: 628 |
| (Chothia) | | |
| Consensus | AAX₁, where X₁ = Y or S | |
| CDRL2 | | |
| (Chothia) | | |
| Consensus | SYX₁X₂PX₃, where X₁ = S or R, X₂ = S or T, and | |
| CDRL3 | X₃ = L or F | |
| (Chothia) | | |
| Consensus | GFX₁FSX₂AW, where X₁ = T or I, and | SEQ ID |
| CDRH1 (IMGT) | X₂ = Y, N, or T | NO: 631 |
| Consensus | IKX₁X₂X₃X₄GGTT, where X₁ = S or R, X₂ = E or K, | SEQ ID |
| CDRH2 (IMGT) | X₃ = T, S, or I, and X₄ = D or Y | NO: 632 |
| Consensus | TTGSSWX₁X₂YX₃FDX₄, where X₁ = N, H, S or Y, | SEQ ID |
| CDRH3 (IMGT) | X₂ = V or L, X₃ = F or Y, and X₄ = F or Y | NO: 633 |
| Consensus | QSX₁SX₂X₃, where X₁ = I or M, X₂ = N, S, R or T, | |
| CDRL1 (IMGT) | and X₃ = Y or H | |
| Consensus | AAX₁, where X₁ = Y or S | |
| CDRL2 (IMGT) | | |
| Consensus | QQSYX₁X₂PX₃T, where X₁ = S or R, X₂ = S or T, | SEQ ID |
| CDRL3 (IMGT) | and X₃ = L or F | NO: 636 |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 637 |
| hIgG1* | NHKPSNTKVDKKVEPKSCDKTHTCPPCPAPELLGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 638 |
| hIgG4 | DHKPSNTKVDKRVESKYGPPCPSCPAPEFLGGPSVFLFPPKPK | |
| | DTLMISRTPEVTCVVVDVSQEDPEVQFNWYVDGVEVHNAK | |
| | TKPREEQFNSTYRVVSVLTVLHQDWLNGKEYKCKVSNKGLP | |
| | SSIEKTISKAKGQPREPQVYTLPPSQEEMTKNQVSLTCLVKGF | |
| | YPSDIAVEWESNGQPENNYKTTPPVLDSDGSFFLYSRLTVDK | |
| | SRWQEGNVFSCSVMHEALHNHYTQKSLSLSLG | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 639 |
| IgG4-SP | DHKPSNTKVDKRVESKYGPPCPPCPAPEFLGGPSVFLFPPKPK | |
| | DTLMISRTPEVTCVVVDVSQEDPEVQFNWYVDGVEVHNAK | |
| | TKPREEQFNSTYRVVSVLTVLHQDWLNGKEYKCKVSNKGLP | |
| | SSIEKTISKAKGQPREPQVYTLPPSQEEMTKNQVSLTCLVKGF | |
| | YPSDIAVEWESNGQPENNYKTTPPVLDSDGSFFLYSRLTVDK | |
| | SRWQEGNVFSCSVMHEALHNHYTQKSLSLSLG | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 640 |
| IgG4-SPLE | DHKPSNTKVDKRVESKYGPPCPPCPAPEELGGPSVFLFPPKPK | |
| | DTLMISRTPEVTCVVVDVSQEDPEVQFNWYVDGVEVHNAK | |
| | TKPREEQFNSTYRVVSVLTVLHQDWLNGKEYKCKVSNKGLP | |
| | SSIEKTISKAKGQPREPQVYTLPPSQEEMTKNQVSLTCLVKGF | |
| | YPSDIAVEWESNGQPENNYKTTPPVLDSDGSFFLYSRLTVDK | |
| | SRWQEGNVFSCSVMHEALHNHYTQKSLSLSLG | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSNFGTQTYTCNV | NO: 641 |
| IgG2 | DHKPSNTKVDKTVERKCCVECPPCPAPPVAGPSVFLFPPKPK | |
| | DTLMISRTPEVTCVVVDVSHEDPEVQFNWYVDGVEVHNAK | |
| | TKPREEQFNSTFRVVSVLTVVHQDWLNGKEYKCKVSNKGLP | |
| | APIEKTISKTKGQPREPQVYTLPPSREEMTKNQVSLTCLVKGF | |
| | YPSDIAVEWESNGQPENNYKTTPPMLDSDGSFFLYSKLTVDK | |
| | SRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 642 |
| hIgG1-N297A | NHKPSNTKVDKKVEPKSCDKTHTCPPCPAPELLGGPSVFLFP | |
| | PKPKDTLMISRTPEVTCVVVDVSHEDPEVKFNWYVDGVEVH | |
| | NAKTKPREEQYASTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 643 |
| hIgG1-D265A | NHKPSNTKVDKKVEPKSCDKTHTCPPCPAPELLGGPSVFLFP | |
| | PKPKDTLMISRTPEVTCVVVAVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 644 |
| hIgG1-LALA | NHKPSNTKVDKKVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 645 |
| hIgG1-LAGA | NHKPSNTKVDKKVEPKSCDKTHTCPPCPAPELAGAPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 646 |
| hIgG1- | NHKPSNTKVDKKVEPKSCDKTHTCPPCPAPEAAGAPSVFLFP | |
| LALAGA | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 647 |
| hIgG1-LALAPG | NHKPSNTKVDKKVEPKSCDKTHTCPPCPAPEAAGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 648 |
| hIgG1-YTE | NHKPSNTKVDKKVEPKSCDKTHTCPPCPAPELLGGPSVFLFP | |
| | PKPKDTLYITREPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 649 |
| hIgG1- | NHKPSNTKVDKKVEPKSCDKTHTCPPCPAPELLGGPSVFLFP | |
| N297A/YTE | PKPKDTLYITREPEVTCVVVDVSHEDPEVKFNWYVDGVEVH | |
| | NAKTKPREEQYASTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 650 |
| hIgG1- | NHKPSNTKVDKKVEPKSCDKTHTCPPCPAPELLGGPSVFLFP | |
| D265A/YTE | PKPKDTLYITREPEVTCVVVAVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 651 |
| hIgG1- | NHKPSNTKVDKKVEPKSCDKTHTCPPCPAPEAAGGPSVFLFP | |
| LALA/YTE | PKPKDTLYITREPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 652 |
| hIgG1- | NHKPSNTKVDKKVEPKSCDKTHTCPPCPAPELAGAPSVFLFP | |
| LAGA/YTE | PKPKDTLYITREPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 653 |
| hIgG1- | NHKPSNTKVDKKVEPKSCDKTHTCPPCPAPEAAGAPSVFLFP | |
| LALAGA/YTE | PKPKDTLYITREPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 654 |
| hIgG1- | NHKPSNTKVDKKVEPKSCDKTHTCPPCPAPEAAGGPSVFLFP | |
| LALAPG/YTE | PKPKDTLYITREPEVTCVVVDVSHEDPEVKFNWYVDGVEVH | |
| | NAKTKPREEQYNSTYRVVSVLTVLHQDWLNGKEYKCKVSN | |
| | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 655 |
| hIgG1-LS | NHKPSNTKVDKKVEPKSCDKTHTCPPCPAPELLGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHSHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 656 |
| hIgG1- | NHKPSNTKVDKKVEPKSCDKTHTCPPCPAPELLGGPSVFLFP | |
| N297A/LS | PKPKDTLMISRTPEVTCVVVDVSHEDPEVKFNWYVDGVEVH | |
| | NAKTKPREEQYASTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHSHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 657 |
| hIgG1- | NHKPSNTKVDKKVEPKSCDKTHTCPPCPAPELLGGPSVFLFP | |
| D265A/LS | PKPKDTLMISRTPEVTCVVVAVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHSHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 658 |
| hIgG1- | NHKPSNTKVDKKVEPKSCDKTHTCPPCPAPEAAGGPSVFLFP | |
| LALA/LS | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHSHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 659 |
| hIgG1- | NHKPSNTKVDKKVEPKSCDKTHTCPPCPAPELAGAPSVFLFP | |
| LAGA/LS | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHSHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 660 |
| hIgG1- | NHKPSNTKVDKKVEPKSCDKTHTCPPCPAPEAAGAPSVFLFP | |
| LALAGA/LS | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHSHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 661 |
| hIgG1- | NHKPSNTKVDKKVEPKSCDKTHTCPPCPAPEAAGGPSVFLFP | |
| LALAPG/LS | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHSHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 662 |
| hIgG1-DHS | NHKPSNTKVDKKVEPKSCDKTHTCPPCPAPELLGGPSVFLFP | |
| | PKPKDTLMISRTPEVTCVVVDVSHEDPEVKFNWYVDGVEVH | |
| | NAKTKPREEQYNSTYRVVSVLTVDHHDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHSHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 663 |
| hIgG1- | NHKPSNTKVDKKVEPKSCDKTHTCPPCPAPELLGGPSVFLFP | |
| N297A/DHS | PKPKDTLMISRTPEVTCVVVDVSHEDPEVKFNWYVDGVEVH | |
| | NAKTKPREEQYASTYRVVSVLTVDHHDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHSHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 664 |
| hIgG1- | NHKPSNTKVDKKVEPKSCDKTHTCPPCPAPELLGGPSVFLFP | |
| D265A/DHS | PKPKDTLMISRTPEVTCVVVAVSHEDPEVKFNWYVDGVEVH | |
| | NAKTKPREEQYNSTYRVVSVLTVDHHDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHSHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 665 |
| hIgG1- | NHKPSNTKVDKKVEPKSCDKTHTCPPCPAPEAAGGPSVFLFP | |
| LALA/DHS | PKPKDTLMISRTPEVTCVVVDVSHEDPEVKFNWYVDGVEVH | |
| | NAKTKPREEQYNSTYRVVSVLTVDHHDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHSHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 666 |
| hIgG1- | NHKPSNTKVDKKVEPKSCDKTHTCPPCPAPELAGAPSVFLFP | |
| LAGA/DHS | PKPKDTLMISRTPEVTCVVVDVSHEDPEVKFNWYVDGVEVH | |
| | NAKTKPREEQYNSTYRVVSVLTVDHHDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHSHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 667 |
| hIgG1- | NHKPSNTKVDKKVEPKSCDKTHTCPPCPAPEAAGAPSVFLFP | |
| LALAGA/DHS | PKPKDTLMISRTPEVTCVVVDVSHEDPEVKFNWYVDGVEVH | |
| | NAKTKPREEQYNSTYRVVSVLTVDHHDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHSHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 668 |
| hIgG1- | NHKPSNTKVDKKVEPKSCDKTHTCPPCPAPEAAGGPSVFLFP | |
| LALAPG/DHS | PKPKDTLMISRTPEVTCVVVDVSHEDPEVKFNWYVDGVEVH | |
| | NAKTKPREEQYNSTYRVVSVLTVDHHDWLNGKEYKCKVSN | |
| | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHSHYTQKSLSLSPG | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 669 |
| hIgG4-YTE | DHKPSNTKVDKRVESKYGPPCPSCPAPEFLGGPSVFLFPPKPK | |
| | DTLYITREPEVTCVVVDVSQEDPEVQFNWYVDGVEVHNAKT | |
| | KPREEQFNSTYRVVSVLTVLHQDWLNGKEYKCKVSNKGLPS | |
| | SIEKTISKAKGQPREPQVYTLPPSQEEMTKNQVSLTCLVKGFY | |
| | PSDIAVEWESNGQPENNYKTTPPVLDSDGSFFLYSRLTVDKS | |
| | RWQEGNVFSCSVMHEALHNHYTQKSLSLSLG | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 670 |
| hIgG4-SP/YTE | DHKPSNTKVDKRVESKYGPPCPPCPAPEFLGGPSVFLFPPKPK | |
| | DTLYITREPEVTCVVVDVSQEDPEVQFNWYVDGVEVHNAKT | |
| | KPREEQFNSTYRVVSVLTVLHQDWLNGKEYKCKVSNKGLPS | |
| | SIEKTISKAKGQPREPQVYTLPPSQEEMTKNQVSLTCLVKGFY | |
| | PSDIAVEWESNGQPENNYKTTPPVLDSDGSFFLYSRLTVDKS | |
| | RWQEGNVFSCSVMHEALHNHYTQKSLSLSLG | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 671 |
| hIgG4- | DHKPSNTKVDKRVESKYGPPCPPCPAPEELGGPSVFLFPPKPK | |
| SPLE/YTE | DTLYITREPEVTCVVVDVSQEDPEVQFNWYVDGVEVHNAKT | |
| | KPREEQFNSTYRVVSVLTVLHQDWLNGKEYKCKVSNKGLPS | |
| | SIEKTISKAKGQPREPQVYTLPPSQEEMTKNQVSLTCLVKGFY | |
| | PSDIAVEWESNGQPENNYKTTPPVLDSDGSFFLYSRLTVDKS | |
| | RWQEGNVFSCSVMHEALHNHYTQKSLSLSLG | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 672 |
| hIgG4-LS | DHKPSNTKVDKRVESKYGPPCPSCPAPEFLGGPSVFLFPPKPK | |
| | DTLMISRTPEVTCVVVDVSQEDPEVQFNWYVDGVEVHNAK | |
| | TKPREEQFNSTYRVVSVLTVLHQDWLNGKEYKCKVSNKGLP | |
| | SSIEKTISKAKGQPREPQVYTLPPSQEEMTKNQVSLTCLVKGF | |
| | YPSDIAVEWESNGQPENNYKTTPPVLDSDGSFFLYSRLTVDK | |
| | SRWQEGNVFSCSVLHEALHSHYTQKSLSLSLG | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 673 |
| hIgG4-SP/LS | DHKPSNTKVDKRVESKYGPPCPPCPAPEFLGGPSVFLFPPKPK | |
| | DTLMISRTPEVTCVVVDVSQEDPEVQFNWYVDGVEVHNAK | |
| | TKPREEQFNSTYRVVSVLTVLHQDWLNGKEYKCKVSNKGLP | |
| | SSIEKTISKAKGQPREPQVYTLPPSQEEMTKNQVSLTCLVKGF | |
| | YPSDIAVEWESNGQPENNYKTTPPVLDSDGSFFLYSRLTVDK | |
| | SRWQEGNVFSCSVLHEALHSHYTQKSLSLSLG | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 674 |
| hIgG4-SPLE/LS | DHKPSNTKVDKRVESKYGPPCPPCPAPEELGGPSVFLFPPKPK | |
| | DTLMISRTPEVTCVVVDVSQEDPEVQFNWYVDGVEVHNAK | |
| | TKPREEQFNSTYRVVSVLTVLHQDWLNGKEYKCKVSNKGLP | |
| | SSIEKTISKAKGQPREPQVYTLPPSQEEMTKNQVSLTCLVKGF | |
| | YPSDIAVEWESNGQPENNYKTTPPVLDSDGSFFLYSRLTVDK | |
| | SRWQEGNVFSCSVLHEALHSHYTQKSLSLSLG | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 675 |
| hIgG4-DHS | DHKPSNTKVDKRVESKYGPPCPSCPAPEFLGGPSVFLFPPKPK | |
| | DTLMISRTPEVTCVVVDVSQEDPEVQFNWYVDGVEVHNAK | |
| | TKPREEQFNSTYRVVSVLTVDHHDWLNGKEYKCKVSNKGL | |
| | PSSIEKTISKAKGQPREPQVYTLPPSQEEMTKNQVSLTCLVKG | |
| | FYPSDIAVEWESNGQPENNYKTTPPVLDSDGSFFLYSRLTVD | |
| | KSRWQEGNVFSCSVMHEALHSHYTQKSLSLSLG | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 676 |
| hIgG4-SP/DHS | DHKPSNTKVDKRVESKYGPPCPPCPAPEFLGGPSVFLFPPKPK | |
| | DTLMISRTPEVTCVVVDVSQEDPEVQFNWYVDGVEVHNAK | |
| | TKPREEQFNSTYRVVSVLTVDHHDWLNGKEYKCKVSNKGL | |
| | PSSIEKTISKAKGQPREPQVYTLPPSQEEMTKNQVSLTCLVKG | |
| | FYPSDIAVEWESNGQPENNYKTTPPVLDSDGSFFLYSRLTVD | |
| | KSRWQEGNVFSCSVMHEALHSHYTQKSLSLSLG | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 677 |
| hIgG4- | DHKPSNTKVDKRVESKYGPPCPPCPAPEELGGPSVFLFPPKPK | |
| SPLE/DHS | DTLMISRTPEVTCVVVDVSQEDPEVQFNWYVDGVEVHNAK | |
| | TKPREEQFNSTYRVVSVLTVDHHDWLNGKEYKCKVSNKGL | |
| | PSSIEKTISKAKGQPREPQVYTLPPSQEEMTKNQVSLTCLVKG | |
| | FYPSDIAVEWESNGQPENNYKTTPPVLDSDGSFFLYSRLTVD | |
| | KSRWQEGNVFSCSVMHEALHSHYTQKSLSLSLG | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSNFGTQTYTCNV | NO: 678 |
| hIgG2-YTE | DHKPSNTKVDKTVERKCCVECPPCPAPPVAGPSVFLFPPKPK | |
| | DTLYITREPEVTCVVVDVSHEDPEVQFNWYVDGVEVHNAKT | |
| | KPREEQFNSTFRVVSVLTVVHQDWLNGKEYKCKVSNKGLPA | |
| | PIEKTISKTKGQPREPQVYTLPPSREEMTKNQVSLTCLVKGFY | |
| | PSDIAVEWESNGQPENNYKTTPPMLDSDGSFFLYSKLTVDKS | |
| | RWQQGNVFSCSVMHEALHNHYTQKSLSLSPG | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSNFGTQTYTCNV | NO: 679 |
| hIgG2-LS | DHKPSNTKVDKTVERKCCVECPPCPAPPVAGPSVFLFPPKPK | |
| | DTLMISRTPEVTCVVVDVSHEDPEVQFNWYVDGVEVHNAK | |
| | TKPREEQFNSTFRVVSVLTVVHQDWLNGKEYKCKVSNKGLP | |
| | APIEKTISKTKGQPREPQVYTLPPSREEMTKNQVSLTCLVKGF | |
| | YPSDIAVEWESNGQPENNYKTTPPMLDSDGSFFLYSKLTVDK | |
| | SRWQQGNVFSCSVLHEALHSHYTQKSLSLSPG | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSNFGTQTYTCNV | NO: 680 |
| hIgG2-DHS | DHKPSNTKVDKTVERKCCVECPPCPAPPVAGPSVFLFPPKPK | |
| | DTLMISRTPEVTCVVVDVSHEDPEVQFNWYVDGVEVHNAK | |
| | TKPREEQFNSTFRVVSVLTVDHHDWLNGKEYKCKVSNKGLP | |
| | APIEKTISKTKGQPREPQVYTLPPSREEMTKNQVSLTCLVKGF | |
| | YPSDIAVEWESNGQPENNYKTTPPMLDSDGSFFLYSKLTVDK | |
| | SRWQQGNVFSCSVMHEALHSHYTQKSLSLSPG | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 681 |
| IgG4-SP | DHKPSNTKVDKRVESKYGPPCPPCPAPEFLGGPSVFLFPPKPK | |
| | DTLMISRTPEVTCVVVDVSQEDPEVQFNWYVDGVEVHNAK | |
| | TKPREEQFNSTYRVVSVLTVLHQDWLNGKEYKCKVSNKGLP | |
| | SSIEKTISKAKGQPREPQVYTLPPSQEEMTKNQVSLTCLVKGF | |
| | YPSDIAVEWESNGQPENNYKTTPPVLDSDGSFFLYSRLTVDK | |
| | SRWQEGNVFSCSVMHEALHNHYTQKSLSLSLG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 682 |
| hIgG1-LA | NHKPSNTKVDKKVEPKSCDKTHTCPPCPAPELLGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHAHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 683 |
| hIgG1- | NHKPSNTKVDKKVEPKSCDKTHTCPPCPAPELLGGPSVFLFP | |
| N297A/LA | PKPKDTLMISRTPEVTCVVVDVSHEDPEVKFNWYVDGVEVH | |
| | NAKTKPREEQYASTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHAHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 684 |
| hIgG1- | NHKPSNTKVDKKVEPKSCDKTHTCPPCPAPELLGGPSVFLFP | |
| D265A/LA | PKPKDTLMISRTPEVTCVVVAVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHAHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 685 |
| hIgG1- | NHKPSNTKVDKKVEPKSCDKTHTCPPCPAPEAAGGPSVFLFP | |
| LALA/LA | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHAHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 686 |
| hIgG1- | NHKPSNTKVDKKVEPKSCDKTHTCPPCPAPELAGAPSVFLFP | |
| LAGA/LA | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHAHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 687 |
| hIgG1- | NHKPSNTKVDKKVEPKSCDKTHTCPPCPAPEAAGAPSVFLFP | |
| LALAGA/LA | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHAHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 688 |
| hIgG1- | NHKPSNTKVDKKVEPKSCDKTHTCPPCPAPEAAGGPSVFLFP | |
| LALAPG/LA | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHAHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 689 |
| hIgG1-N434A | NHKPSNTKVDKKVEPKSCDKTHTCPPCPAPELLGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHAHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 690 |
| hIgG1-N297A/ | NHKPSNTKVDKKVEPKSCDKTHTCPPCPAPELLGGPSVFLFP | |
| N434A | PKPKDTLMISRTPEVTCVVVDVSHEDPEVKFNWYVDGVEVH | |
| | NAKTKPREEQYASTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHAHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 691 |
| hIgG1-D265A/ | NHKPSNTKVDKKVEPKSCDKTHTCPPCPAPELLGGPSVFLFP | |
| N434A | PKPKDTLMISRTPEVTCVVVAVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHAHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 692 |
| hIgG1-LALA/ | NHKPSNTKVDKKVEPKSCDKTHTCPPCPAPEAAGGPSVFLFP | |
| N434A | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHAHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 693 |
| hIgG1-LAGA/ | NHKPSNTKVDKKVEPKSCDKTHTCPPCPAPELAGAPSVFLFP | |
| N434A | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHAHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 694 |
| hIgG1- | NHKPSNTKVDKKVEPKSCDKTHTCPPCPAPEAAGAPSVFLFP | |
| LALAGA/ | PKPKDTLMISRTPEVTCVVVDVSHEDPEVKFNWYVDGVEVH | |
| N434A | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHAHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 695 |
| hIgG1- | NHKPSNTKVDKKVEPKSCDKTHTCPPCPAPEAAGGPSVFLFP | |
| LALAPG/ | PKPKDTLMISRTPEVTCVVVDVSHEDPEVKFNWYVDGVEVH | |
| N434A | NAKTKPREEQYNSTYRVVSVLTVLHQDWLNGKEYKCKVSN | |
| | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHAHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 696 |
| hIgG1-N434W | NHKPSNTKVDKKVEPKSCDKTHTCPPCPAPELLGGPSVFLFP | |
| | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHWHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 697 |
| hIgG1-N297A/ | NHKPSNTKVDKKVEPKSCDKTHTCPPCPAPELLGGPSVFLFP | |
| N434W | PKPKDTLMISRTPEVTCVVVDVSHEDPEVKFNWYVDGVEVH | |
| | NAKTKPREEQYASTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHWHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 698 |
| hIgG1-D265A/ | NHKPSNTKVDKKVEPKSCDKTHTCPPCPAPELLGGPSVFLFP | |
| N434W | PKPKDTLMISRTPEVTCVVVAVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHWHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 699 |
| hIgG1-LALA/ | NHKPSNTKVDKKVEPKSCDKTHTCPPCPAPEAAGGPSVFLFP | |
| N434W | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHWHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 700 |
| hIgG1-LAGA/ | NHKPSNTKVDKKVEPKSCDKTHTCPPCPAPELAGAPSVFLFP | |
| N434W | PKPKDTLMISRTPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHWHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 701 |
| hIgG1- | NHKPSNTKVDKKVEPKSCDKTHTCPPCPAPEAAGAPSVFLFP | |
| LALAGA/ | PKPKDTLMISRTPEVTCVVVDVSHEDPEVKFNWYVDGVEVH | |
| N434W | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHWHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 702 |
| hIgG1- | NHKPSNTKVDKKVEPKSCDKTHTCPPCPAPEAAGGPSVFLFP | |
| LALAPG/ | PKPKDTLMISRTPEVTCVVVDVSHEDPEVKFNWYVDGVEVH | |
| N434W | NAKTKPREEQYNSTYRVVSVLTVLHQDWLNGKEYKCKVSN | |
| | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHWHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 703 |
| hIgG1-DQ | NHKPSNTKVDKKVEPKSCDKTHTCPPCPAPELLGGPSVFLFP | |
| | PKPKDTLMISRDPEVTCVVVDVSHEDPEVKFNWYVDGVEVH | |
| | NAKTKPREEQYNSTYRVVSVLQVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 704 |
| hIgG1- | NHKPSNTKVDKKVEPKSCDKTHTCPPCPAPELLGGPSVFLFP | |
| N297A/DQ | PKPKDTLMISRDPEVTCVVVDVSHEDPEVKFNWYVDGVEVH | |
| | NAKTKPREEQYASTYRVVSVLQVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 705 |
| hIgG1- | NHKPSNTKVDKKVEPKSCDKTHTCPPCPAPELLGGPSVFLFP | |
| D265A/DQ | PKPKDTLMISRDPEVTCVVVAVSHEDPEVKFNWYVDGVEVH | |
| | NAKTKPREEQYNSTYRVVSVLQVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 706 |
| hIgG1- | NHKPSNTKVDKKVEPKSCDKTHTCPPCPAPEAAGGPSVFLFP | |
| LALA/DQ | PKPKDTLMISRDPEVTCVVVDVSHEDPEVKFNWYVDGVEVH | |
| | NAKTKPREEQYNSTYRVVSVLQVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 707 |
| hIgG1- | NHKPSNTKVDKKVEPKSCDKTHTCPPCPAPELAGAPSVFLFP | |
| LAGA/DQ | PKPKDTLMISRDPEVTCVVVDVSHEDPEVKFNWYVDGVEVH | |
| | NAKTKPREEQYNSTYRVVSVLQVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 708 |
| hIgG1- | NHKPSNTKVDKKVEPKSCDKTHTCPPCPAPEAAGAPSVFLFP | |
| LALAGA/DQ | PKPKDTLMISRDPEVTCVVVDVSHEDPEVKFNWYVDGVEVH | |
| | NAKTKPREEQYNSTYRVVSVLQVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 709 |
| hIgG1- | NHKPSNTKVDKKVEPKSCDKTHTCPPCPAPEAAGGPSVFLFP | |
| LALAPG/DQ | PKPKDTLMISRDPEVTCVVVDVSHEDPEVKFNWYVDGVEVH | |
| | NAKTKPREEQYNSTYRVVSVLQVLHQDWLNGKEYKCKVSN | |
| | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 710 |
| hIgG1-DW | NHKPSNTKVDKKVEPKSCDKTHTCPPCPAPELLGGPSVFLFP | |
| | PKPKDTLMISRDPEVTCVVVDVSHEDPEVKFNWYVDGVEVH | |
| | NAKTKPREEQYNSTYRVVSVLWVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 711 |
| hIgG1- | NHKPSNTKVDKKVEPKSCDKTHTCPPCPAPELLGGPSVFLFP | |
| N297A/DW | PKPKDTLMISRDPEVTCVVVDVSHEDPEVKFNWYVDGVEVH | |
| | NAKTKPREEQYASTYRVVSVLWVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 712 |
| hIgG1- | NHKPSNTKVDKKVEPKSCDKTHTCPPCPAPELLGGPSVFLFP | |
| D265A/DW | PKPKDTLMISRDPEVTCVVVAVSHEDPEVKFNWYVDGVEVH | |
| | NAKTKPREEQYNSTYRVVSVLWVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 713 |
| hIgG1- | NHKPSNTKVDKKVEPKSCDKTHTCPPCPAPEAAGGPSVFLFP | |
| LALA/DW | PKPKDTLMISRDPEVTCVVVDVSHEDPEVKFNWYVDGVEVH | |
| | NAKTKPREEQYNSTYRVVSVLWVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 714 |
| hIgG1- | NHKPSNTKVDKKVEPKSCDKTHTCPPCPAPELAGAPSVFLFP | |
| LAGA/DW | PKPKDTLMISRDPEVTCVVVDVSHEDPEVKFNWYVDGVEVH | |
| | NAKTKPREEQYNSTYRVVSVLWVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 715 |
| hIgG1- | NHKPSNTKVDKKVEPKSCDKTHTCPPCPAPEAAGAPSVFLFP | |
| LALAGA/DW | PKPKDTLMISRDPEVTCVVVDVSHEDPEVKFNWYVDGVEVH | |
| | NAKTKPREEQYNSTYRVVSVLWVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 716 |
| hIgG1- | NHKPSNTKVDKKVEPKSCDKTHTCPPCPAPEAAGGPSVFLFP | |
| LALAPG/DW | PKPKDTLMISRDPEVTCVVVDVSHEDPEVKFNWYVDGVEVH | |
| | NAKTKPREEQYNSTYRVVSVLWVLHQDWLNGKEYKCKVSN | |
| | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 717 |
| hIgG1-YD | NHKPSNTKVDKKVEPKSCDKTHTCPPCPAPELLGGPSVFLFP | |
| | PKPKDTLYISRDPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 718 |
| hIgG1- | NHKPSNTKVDKKVEPKSCDKTHTCPPCPAPELLGGPSVFLFP | |
| N297A/YD | PKPKDTLYISRDPEVTCVVVDVSHEDPEVKFNWYVDGVEVH | |
| | NAKTKPREEQYASTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 719 |
| hIgG1- | NHKPSNTKVDKKVEPKSCDKTHTCPPCPAPELLGGPSVFLFP | |
| D265A/YD | PKPKDTLYISRDPEVTCVVVAVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 720 |
| hIgG1- | NHKPSNTKVDKKVEPKSCDKTHTCPPCPAPEAAGGPSVFLFP | |
| LALA/YD | PKPKDTLYISRDPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 721 |
| hIgG1- | NHKPSNTKVDKKVEPKSCDKTHTCPPCPAPELAGAPSVFLFP | |
| LAGA/YD | PKPKDTLYISRDPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 722 |
| hIgG1- | NHKPSNTKVDKKVEPKSCDKTHTCPPCPAPEAAGAPSVFLFP | |
| LALAGA/YD | PKPKDTLYISRDPEVTCVVVDVSHEDPEVKFNWYVDGVEVH | |
| | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 723 |
| hIgG1- | NHKPSNTKVDKKVEPKSCDKTHTCPPCPAPEAAGGPSVFLFP | |
| LALAPG/YD | PKPKDTLYISRDPEVTCVVVDVSHEDPEVKFNWYVDGVEVH | |
| | NAKTKPREEQYNSTYRVVSVLTVLHQDWLNGKEYKCKVSN | |
| | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVT | SEQ ID |
| Constant Region | VSWNSGALTSGVHTFPAVLQSSGLYSLSSVVTVPSSSLGTQT | NO: 724 |
| hIgG1-QVV | YICNVNHKPSNTKVDKKVEPKSCDKTHTCPPCPAPELLGGPS | |
| | VFLFPPKPKDTLMISRTPEVTCVVVDVSHEDPEVKFNWYVD | |
| | GVEVHNAKTKPREEQYNSTYRVVSVLQVLHVDWLNGKEYK | |
| | CKVSNKALPAPIEKTISKAKGQPREPQVYTLPPSRDELTKNQV | |
| | SLTCLVKGFYPSDIVVEWESNGQPENNYKTTPPVLDSDGSFF | |
| | LYSKLTVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 725 |
| hIgG1- | NHKPSNTKVDKKVEPKSCDKTHTCPPCPAPELLGGPSVFLFP | |
| N297A/QVV | PKPKDTLMISRTPEVTCVVVDVSHEDPEVKFNWYVDGVEVH | |
| | NAKTKPREEQYASTYRVVSVLQVLHVDWLNGKEYKCKVSN | |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 726 |
| hIgG1- | NHKPSNTKVDKKVEPKSCDKTHTCPPCPAPELLGGPSVFLFP | |
| D265A/QVV | PKPKDTLMISRTPEVTCVVVAVSHEDPEVKFNWYVDGVEVH | |
| | NAKTKPREEQYNSTYRVVSVLQVLHVDWLNGKEYKCKVSN | |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 727 |
| hIgG1- | NHKPSNTKVDKKVEPKSCDKTHTCPPCPAPEAAGGPSVFLFP | |
| LALA/QVV | PKPKDTLMISRTPEVTCVVVDVSHEDPEVKFNWYVDGVEVH | |
| | NAKTKPREEQYNSTYRVVSVLQVLHVDWLNGKEYKCKVSN | |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 728 |
| hIgG1- | NHKPSNTKVDKKVEPKSCDKTHTCPPCPAPELAGAPSVFLFP | |
| LAGA/QVV | PKPKDTLMISRTPEVTCVVVDVSHEDPEVKFNWYVDGVEVH | |
| | NAKTKPREEQYNSTYRVVSVLQVLHVDWLNGKEYKCKVSN | |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 729 |
| hIgG1- | NHKPSNTKVDKKVEPKSCDKTHTCPPCPAPEAAGAPSVFLFP | |
| LALAGA/QVV | PKPKDTLMISRTPEVTCVVVDVSHEDPEVKFNWYVDGVEVH | |
| | NAKTKPREEQYNSTYRVVSVLQVLHVDWLNGKEYKCKVSN | |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 730 |
| hIgG1- | NHKPSNTKVDKKVEPKSCDKTHTCPPCPAPEAAGGPSVFLFP | |
| LALAPG/QVV | PKPKDTLMISRTPEVTCVVVDVSHEDPEVKFNWYVDGVEVH | |
| | NAKTKPREEQYNSTYRVVSVLQVLHVDWLNGKEYKCKVSN | |
| | KALGAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 731 |
| hIgG1-DDRVV | NHKPSNTKVDKKVEPKSCDKTHTCPPCPAPELLGGPSVFLFP | |
| | PKPKDTLMISRDPEVTCVVVDVSHEDPEVKFNWYVDGVEVD | |
| | NAKTKPREEQYNSTYRVVSVLRVLHVDWLNGKEYKCKVSN | |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 732 |
| hIgG1- | NHKPSNTKVDKKVEPKSCDKTHTCPPCPAPELLGGPSVFLFP | |
| N297A/DDRVV | PKPKDTLMISRDPEVTCVVVDVSHEDPEVKFNWYVDGVEVD | |
| | NAKTKPREEQYASTYRVVSVLRVLHVDWLNGKEYKCKVSN | |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 733 |
| hIgG1- | NHKPSNTKVDKKVEPKSCDKTHTCPPCPAPELLGGPSVFLFP | |
| D265A/DDRVV | PKPKDTLMISRDPEVTCVVVAVSHEDPEVKFNWYVDGVEVD | |
| | NAKTKPREEQYNSTYRVVSVLRVLHVDWLNGKEYKCKVSN | |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 734 |
| hIgG1- | NHKPSNTKVDKKVEPKSCDKTHTCPPCPAPEAAGGPSVFLFP | |
| LALA/DDRVV | PKPKDTLMISRDPEVTCVVVDVSHEDPEVKFNWYVDGVEVD | |
| | NAKTKPREEQYNSTYRVVSVLRVLHVDWLNGKEYKCKVSN | |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 735 |
| hIgG1- | NHKPSNTKVDKKVEPKSCDKTHTCPPCPAPELAGAPSVFLFP | |
| LAGA/DDRVV | PKPKDTLMISRDPEVTCVVVDVSHEDPEVKFNWYVDGVEVD | |
| | NAKTKPREEQYNSTYRVVSVLRVLHVDWLNGKEYKCKVSN | |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 736 |
| hIgG1- | NHKPSNTKVDKKVEPKSCDKTHTCPPCPAPEAAGAPSVFLFP | |
| LALAGA/DDRVV | PKPKDTLMISRDPEVTCVVVDVSHEDPEVKFNWYVDGVEVD | |
| | NAKTKPREEQYNSTYRVVSVLRVLHVDWLNGKEYKCKVSN | |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 737 |
| hIgG1- | NHKPSNTKVDKKVEPKSCDKTHTCPPCPAPEAAGGPSVFLFP | |
| LALAPG/DDRVV | PKPKDTLMISRDPEVTCVVVDVSHEDPEVKFNWYVDGVEVD | |
| | NAKTKPREEQYNSTYRVVSVLRVLHVDWLNGKEYKCKVSN | |
| | KALGAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPG | |
| Human kappa | RTVAAPSVFIFPPSDEQLKSGTASVVCLLNNFYPREAKVQWK | SEQ ID |
| LC | VDNALQSGNSQESVTEQDSKDSTYSLSSTLTLSKADYEKHKV | NO: 738 |
| | YACEVTHQGLSSPVTKSFNRGEC | |
| OX40L protein | MERVQPLEENVGNAARPRFERNKLLLVASVIQGLGLLLCFTY | SEQ ID |
| | ICLHFSALQVSHRYPRIQSIKVQFTEYKKEKGFILTSQKEDEIM | NO: 739 |
| | KVQNNSVIINCDGFYLISLKGYFSQEVNISLHYQKDEEPLFQL | |
| | KKVRSVNSLMVASLTYKDKVYLNVTTDNTSLDDFHVNGGE | |
| | LILIHQNPGEFCVL | |
| Kabat HCDR1 | SGNWWS | SEQ ID |
| | | NO: 740 |
| Kabat HCDR1 | SGGYYWS | SEQ ID |
| | | NO: 741 |
| Chothia HCDR1 | GGSISSGN | SEQ ID |
| | | NO: 742 |
| Chothia HCDR1 | GFTFDSA | SEQ ID |
| | | NO: 743 |
| Chothia HCDR1 | GGSISSGGY | SEQ ID |
| | | NO: 744 |
| IMGT CDR1 | GGSISSGNW | SEQ ID |
| | | NO: 745 |
| IMGT CDR1 | GFTFDSAW | SEQ ID |
| | | NO: 746 |
| IMGT CDR1 | GGSISSGGYY | SEQ ID |
| | | NO: 747 |
| Kabat HCDR2 | RFKSETDGGTKDFGAHVRG | SEQ ID |
| | | NO: 748 |
| Kabat HCDR2 | EIHHSGRTNYNPSLKS | SEQ ID |
| | | NO: 749 |
| Kabat HCDR2 | RIKRKTDGGTTDYAAPVKG | SEQ ID |
| | | NO: 750 |
| Kabat HCDR2 | RIKSKNEGGTIDYAAPVKG | SEQ ID |
| | | NO: 751 |
| Kabat HCDR2 | WIHPNTGKTGYAQKFQG | SEQ ID |
| | | NO: 752 |
| Kabat HCDR2 | HIYYSGSTKYNPSLKS | SEQ ID |
| | | NO: 753 |
| Chothia HCDR2 | HHSGR | SEQ ID |
| | | NO: 754 |
| Chothia HCDR2 | KSKNEGGT | SEQ ID |
| | | NO: 755 |
| Chothia HCDR2 | HPNTGK | SEQ ID |
| | | NO: 756 |
| Chothia HCDR2 | YYSGS | SEQ ID |
| | | NO: 757 |
| IMGT HCDR2 | FKSETDGGTK | SEQ ID |
| | | NO: 758 |
| IMGT HCDR2 | IHHSGRT | SEQ ID |
| | | NO: 759 |
| IMGT HCDR2 | IKSKNEGGTI | SEQ ID |
| | | NO: 760 |
| IMGT HCDR2 | IHPNTGKT | SEQ ID |
| | | NO: 761 |
| IMGT HCDR2 | IYYSGST | SEQ ID |
| | | NO: 762 |
| Kabat HCDR3 | GGTAVPFDF | SEQ ID |
| | | NO: 763 |
| Kabat HCDR3 | DRQGSFAFDI | SEQ ID |
| | | NO: 764 |
| Kabat HCDR3 | DRPWGSFFDY | SEQ ID |
| | | NO: 765 |
| Kabat HCDR3 | DFLWFGELPFDY | SEQ ID |
| | | NO: 766 |
| Kabat HCDR3 | ESYDLWSGHSFNWFDP | SEQ ID |
| | | NO: 767 |
| Kabat HCDR3 | DNWGSGWYYMDV | SEQ ID |
| | | NO: 768 |
| Chothia HCDR3 | GTAVPFD | SEQ ID |
| | | NO: 769 |
| Chothia HCDR3 | RQGSFAFD | SEQ ID |
| | | NO: 770 |
| Chothia HCDR3 | RPWGSFFD | SEQ ID |
| | | NO: 771 |
| Chothia HCDR3 | FLWFGELPFD | SEQ ID |
| | | NO: 772 |
| Chothia HCDR3 | SYDLWSGHSFNWFD | SEQ ID |
| | | NO: 773 |
| Chothia HCDR3 | NWGSGWYYMD | SEQ ID |
| | | NO: 774 |
| IMGT HCDR3 | TTGGTAVPFDF | SEQ ID |
| | | NO: 775 |
| IMGT HCDR3 | ARDRQGSFAFDI | SEQ ID |
| | | NO: 776 |
| IMGT HCDR3 | TLDRPWGSFFDY | SEQ ID |
| | | NO: 777 |
| IMGT HCDR3 | NTDFLWFGELPFDY | SEQ ID |
| | | NO: 778 |
| IMGT HCDR3 | ARESYDLWSGHSFNWFDP | SEQ ID |
| | | NO: 779 |
| IMGT HCDR3 | ARDNWGSGWYYMDV | SEQ ID |
| | | NO: 780 |
| Kabat LCDR1 | KSSQSLLYSSNNKNYLA | SEQ ID |
| | | NO: 781 |
| Kabat LCDR1 | RASQSISTWLV | SEQ ID |
| | | NO: 782 |
| Kabat LCDR1 | KSSQSVLYSSNNKNYFA | SEQ ID |
| | | NO: 783 |
| Kabat LCDR1 | RASQGISNHLA | SEQ ID |
| | | NO: 784 |
| Kabat LCDR1 | RASQGITSYLA | SEQ ID |
| | | NO: 785 |
| Kabat LCDR1 | RASQSVSNYFA | SEQ ID |
| | | NO: 786 |
| Chothia LCDR1 | SQSLLYSSNNKNY | SEQ ID |
| | | NO: 787 |
| Chothia LCDR1 | SQSISTW | SEQ ID |
| | | NO: 788 |
| Chothia LCDR1 | SQGISNH | SEQ ID |
| | | NO: 789 |
| Chothia LCDR1 | SQGITSY | SEQ ID |
| | | NO: 790 |
| Chothia LCDR1 | SQSVSNY | SEQ ID |
| | | NO: 791 |
| IMGT LCDR1 | QSLLYSSNNKNY | SEQ ID |
| | | NO: 792 |
| IMGT LCDR1 | QSISTW | SEQ ID |
| | | NO: 793 |
| IMGT LCDR1 | QGISNH | SEQ ID |
| | | NO: 794 |
| IMGT LCDR1 | QGITSY | SEQ ID |
| | | NO: 795 |
| IMGT LCDR1 | QSVSNY | SEQ ID |
| | | NO: 796 |
| Kabat LCDR2 | WTSTRES | SEQ ID |
| | | NO: 797 |
| Kabat LCDR2 | KASSLES | SEQ ID |
| | | NO: 798 |
| Kabat LCDR2 | RASTRES | SEQ ID |
| | | NO: 799 |
| Kabat LCDR2 | GATTLQS | SEQ ID |
| | | NO: 800 |
| Kabat LCDR2 | DASNRAT | SEQ ID |
| | | NO: 801 |
| Chothia and | WTS | |
| IMGT LCDR2 | | |
| Chothia and | KAS | |
| IMGT LCDR2 | | |
| Chothia and | RAS | |
| IMGT LCDR2 | | |
| Chothia and | GAT | |
| IMGT LCDR2 | | |
| Kabat LCDR3 | LQYSNTPYS | SEQ ID |
| | | NO: 806 |
| Kabat LCDR3 | QQHISYPYT | SEQ ID |
| | | NO: 807 |
| Kabat LCDR3 | QQYYTTPLT | SEQ ID |
| | | NO: 808 |
| Kabat LCDR3 | QKLNSAPLT | SEQ ID |
| | | NO: 809 |
| Kabat LCDR3 | QQLQSYPYT | SEQ ID |
| | | NO: 810 |
| Kabat LCDR3 | QHRSNWPLT | SEQ ID |
| | | NO: 811 |
| Chothia and | YSNTPY | SEQ ID |
| IMGT LCDR3 | | NO: 812 |
| Chothia and | HISYPY | SEQ ID |
| IMGT LCDR3 | | NO: 813 |
| Chothia and | YYTTPL | SEQ ID |
| IMGT LCDR3 | | NO: 814 |
| Chothia and | LNSAPL | SEQ ID |
| IMGT LCDR3 | | NO: 815 |
| Chothia and | LQSYPY | SEQ ID |
| IMGT LCDR3 | | NO: 816 |
| Chothia and | RSNWPL | SEQ ID |
| IMGT LCDR3 | | NO: 817 |
| Heavy Chain | EVQLVESGGGLVKPGGSLRLSCAASGFTFSNAWMSWVRQAP | SEQ ID |
| Variable Domain | GKGLEWVGRFKSETDGGTKDFGAHVRGRFIISKDASKNTLY | NO: 818 |
| 71 | LQMNRLKTEDTAVYYCTTGGTAVPFDFWGLGTLVTVSS | |
| Heavy Chain | QVQLQESGPGLVKPSGTLSLTCAVSGGSISSGNWWSWVRQP | SEQ ID |
| Variable Domain | PGSGLEWIGEIHHSGRTNYNPSLKSRVVISVDKSKNHFSLKLS | NO: 819 |
| 72 | SVTAADTAVYYCARDRQGSFAFDIWGQGTMVTVSS | |
| Heavy Chain | EVQLVESGGGLVKPGGSLRLSCTASGFTFDSAWMSWVRQAP | SEQ ID |
| Variable Domain | GKGLEWVGRIKRKTDGGTTDYAAPVKGRFTISRDDSTKTLY | NO: 820 |
| 73 | LQMNSLKTEDTAVYYCTLDRPWGSFFDYWGQGTLVTVSS | |
| Heavy Chain | EVQLVESGGGLVKPGGSLRLSCAASGFTFSNAWMSWVRQAP | SEQ ID |
| Variable Domain | GKGLEWVGRIKSKNEGGTIDYAAPVKGRFTISRDDSKNTLYL | NO: 821 |
| 74 | QMNSLKTEDTAVYYCNTDFLWFGELPFDYWGQGTLVTVSS | |
| Heavy Chain | EVQVVESGGGLVQPGGSLRLSCAASGFTFSSYAMTWVRQAP | SEQ ID |
| Variable Domain | GKGLEWVSLISGSGGLTKYADSVKGRFTISRDNSKKTLYLQ | NO: 822 |
| 75 | MNSLRAEDTAAYYCVKDEGLTTGEYWGQGTLVTVSS | |
| Heavy Chain | QVQLVQSGTEVKKPGASVKVSCKASGYTFTNYDINWVRQA | SEQ ID |
| Variable Domain | TGQGLEWVGWIHPNTGKTGYAQKFQGRVTMTRDISIGTAY | NO: 823 |
| 76 | MELNSLRSEDTAVYYCARESYDLWSGHSFNWFDPWGQGTL | |
| | VTVSS | |
| Heavy Chain | QVQLQESGPGLVKPSQTLSLTCTVSGGSISSGGYYWSWIRQY | SEQ ID |
| Variable Domain | PGKGLEWIGHIYYSGSTKYNPSLKSRLTISVDTSKNQFSLKLS | NO: 824 |
| 77 | SVTAADTAVYYCARDNWGSGWYYMDVWGKGTTVTVSS | |
| Light Chain | DIVMTQSPDSLAVSLGERATINCKSSQSLLYSSNNKNYLAWY | SEQ ID |
| Variable Domain | QQKPGQPPKLLFYWTSTRESGVPDRFGASGSGTDFTLTISSLQ | NO: 825 |
| 57 | VEDVAIYFCLQYSNTPYSFGQGTKLEIK | |
| Light Chain | DIQMTQSPATLSASVGDRVTITCRASQSISTWLVWYQQKPGK | SEQ ID |
| Variable Domain | APKLLIYKASSLESGVPSRFSGSGSGTEFTLTISSLQPDDFATY | NO: 826 |
| 58 | YCQQHISYPYTFGQGTRLEIK | |
| Light Chain | DIVMTQSPDSLAVSLGERATINCKSSQSVLYSSNNKNYFAWY | SEQ ID |
| Variable Domain | QQKPGQPPRLLIYRASTRESGVPDRFSGSGSGTDFTLTISSLQA | NO: 827 |
| 59 | EDVAVYYCQQYYTTPLTFGGGTKVEIR | |
| Light Chain | DNQMTQSPSSLSASVGDRVTITCRASQGISNHLAWYQQKPG | SEQ ID |
| Variable Domain | KVPDLLIYAASTLQSGVPSRFSGSGSGTDFTLTISSLQPEDVA | NO: 828 |
| 60 | NYYCQKLNSAPLTFGGGTKVEIK | |
| Light Chain | DIQMTQSPSSLSASVGDRVTITCRASQGIRNDLGWYQQKPGK | SEQ ID |
| Variable Domain | APKRLIYAASSLQSGVPSRFSGSGSGTEFTLTISSLQPEDFATY | NO: 829 |
| 61 | YCLQHNNYPFTFGPGTKVDIK | |
| Light Chain | DIQLTQSPSFLSASVGDRVIITCRASQGITSYLAWYQRKPGKA | SEQ ID |
| Variable Domain | PKLLIYGATTLQSGVPSRFSGSGSGTEFTLTISSLQPEDFATYY | NO: 830 |
| 62 | CQQLQSYPYTFGQGTKLEIK | |
| Light Chain | EIVLTQSPATLSLSPGERATLSCRASQSVSNYFAWYRQKPGQ | SEQ ID |
| Variable Domain | APRLLIYDASNRATGIPARFSGSGSGTDFTLTISSLEPEDFAVY | NO: 831 |
| 63 | YCQHRSNWPLTFGGGTKVEIK | |
| Amlitelimab VH | EVQLVESGGGLVQPGGSLRLSCAASGFTFSNYAMNWVRQAP | SEQ ID |
| | GKGLEWVSTISGSGGATRYADSVKGRFTISRDNSRNTVYLQ | NO: 832 |
| | MNSLRVEDTAVFYCTKDRLIMATVRGPYYYGMDVWGQGTT | |
| | VTVSS | |
| Amlitelimab VL | DIQMTQSPSSLSASVGDRVTITCRASQSISSYLNWYQQKPGK | SEQ ID |
| | APNLLIYAASSLQSGVPSRFSGSGSETDFTLTISSLQPEDFATY | NO: 833 |
| | YCQQSHSVSFTFGPGTKVDIK | |
| LC.001 | EVQLLESGGGLVQPGGSLRLSCAASGFTFNSYAMSWVRQAP | SEQ ID |
| (Oxelumab) VH | GKGLEWVSIISGSGGFTYYADSVKGRFTISRDNSRTTLYLQM | NO: 834 |
| | NSLRAEDTAVYYCAKDRLVAPGTFDYWGQGALVTVSS | |
| LC.001 | DIQMTQSPSSLSASVGDRVTITCRASQGISSWLAWYQQKPEK | SEQ ID |
| (Oxelumab) VL | APKSLIYAASSLQSGVPSRFSGSGSGTDFTLTISSLQPEDFATY | NO: 835 |
| | YCQQYNSYPYTFGQGTKLEIK | |
| LC.005 VH | QVQLVESGGGVVQPGRSLRLSCAASGFTFSNFGMHWVRQAP | SEQ ID |
| | GKGLEWVAAIWYDGHDKYYSYYVKGRFTISRDNSKNTLFL | NO: 836 |
| | QMNSLRAEDTAVYYCARDSSSWYRYFDYWGQGTLVTVSS | |
| LC.005 VL | EIVLTQSPGTLSLSPGERATLSCRASQSVSSNYLAWYQQKPG | SEQ ID |
| | QAPRLLIYGASSRATGIPDRFSGSGSGTDFTLTISRLEPEDFAV | NO: 837 |
| | YYCQQYGSSFTFGPGTKVDIK | |
| LC.019 VH | QKQLVEFGGGVVQPGRSLRLSCAASGFTFSNYGMHWVRQA | SEQ ID |
| | PGKGLWVAVIWNDGSNKYYVDSVKGRFIISRDNSKNTLYLQ | NO: 838 |
| | MNSLRAEDTAVYYCARDRMGIYYYGMDVWGQGTTVTVSS | |
| LC.019 VL | EIVLTQSPATLSLSPGERATLSCRASQGVSRYLAWYQQKPGQ | SEQ ID |
| | APRLLIYDASNRATGIPARVSGSGPGTDFTLTISSLEPEDFAVD | NO: 839 |
| | YCQQRSNWQYTFGQGTKLEIK | |
| Chothia HCDR1 | GFTFSNA | SEQ ID |
| | | NO: 840 |
| Heavy Chain | EVRLVESGGGLVQPGGSLRLSCAASGFTFSSYAMTWVRQAP | SEQ ID |
| Variable Domain | GKGLEWVSLISGSGGLTKYADSVKGRFTISRDNSKKTLYLQ | NO: 841 |
| 78 | MNSLRAEDTAVYYCAKDEGLTTGEYWGQGTLVTVSS | |
| Light Chain | DIQMTQSPSSLSASVGDRVTITCRASQGIRNDLGWYQQKPGK | SEQ ID |
| Variable Domain | APKRLIYATSSFQSGVPSRFSGSGSGTEFTLTISSLQPEDFATY | NO: 842 |
| 64 | YCLQHNSYPFTFGPGTKVDVK | |

*SEQ ID NO: 637 is an hIgG1 sequence having a SRDEL (SEQ ID NO: 1016) allotype. The disclosure also encompasses SREEM (SEQ ID NO: 1017) allotype variants of any of the SRDEL (SEQ ID NO: 1016) allotype sequences set forth in the table above, and either a SRDEL (SEQ ID NO: 1016) allotype heavy chain constant region sequence or a SREEM (SEQ ID NO: 1017) allotype heavy chain constant region sequence may be included in an antibody described herein.

SEQ ID NO: 843
MGVLKFKHIFFRSFVKSSGVSQIVFTFLLIPCCLTLNFRAPPVIPNVPFLWAWNAPSEFCLGKFD
EPLDMSLFSFIGSPRINATGQGVTIFYVDRLGYYPYIDSITGVTVNGGIPQKISLQDHLDKAKKD
ITFYMPVDNLGMAVIDWEEWRPTWARNWKPKDVYKNRSIELVQQQNVQLSLTEATEKAKQEFEKA
GKDFLVETIKLGKLLRPNHLWGYYLFPDCYNHHYKKPGYNGSCFNVEIKRNDDLSWLWNESTALY
PSIYLNTQQSPVAATLYVRNRVREAIRVSKIPDAKSPLPVFAYTRIVFTDQVLKFLSQDELVYTF
GETVALGASGIVIWGTLSIMRSMKSCLLLDNYMETILNPYIINVTLAAKMCSQVLCQEQGVCIRK
NWNSSDYLHLNPDNFAIQLEKGGKFTVRGKPTLEDLEQFSEKFYCSCYSTLSCKEKADVKDTDAV
DVCIADGVCIDAFLKPPMETEEPQIFYNASPSTLSATMFIVSILFLIISSVASL
SEQ ID NO: 844
LNFRAPPVIPNVPFLWAWNAPSEFCLGKFDEPLDMSLFSFIGSPRINATGQGVTIFYVDRLGYYP
YIDSITGVTVNGGIPQKISLQDHLDKAKKDITFYMPVDNLGMAVIDWEEWRPTWARNWKPKDVYK
NRSIELVQQQNVQLSLTEATEKAKQEFEKAGKDFLVETIKLGKLLRPNHLWGYYLFPDCYNHHYK
KPGYNGSCFNVEIKRNDDLSWLWNESTALYPSIYLNTQQSPVAATLYVRNRVREAIRVSKIPDAK
SPLPVFAYTRIVFTDQVLKFLSQDELVYTFGETVALGASGIVIWGTLSIMRSMKSCLLLDNYMET
ILNPYIINVTLAAKMCSQVLCQEQGVCIRKNWNSSDYLHLNPDNFAIQLEKGGKFTVRGKPTLED
LEQFSEKFYCSCYSTLSCKEKADVKDTDAVDVCIADGVCIDAFLKPPMETEEPQIFYNASPSTLS
ATMFIVSILFLIISSVASL
SEQ ID NO: 845
LNFRAPPVIPNVPFLWAWNAPSEFCLGKFDEPLDMSLFSFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDFLVETIKLGKLLRPNHLWGYYLFPDCYNHHYKKPGYNG
SCFNVEIKRNDDLSWLWNESTALYPSIYLNTQQSPVAATLYVRNRVREAIRVSKIPDAKSPLPVFAY
TRIVFTDQVLKFLSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDAFLKPPMETEEPQIFY
SEQ ID NO: 846
LNFRAPPVIPNVPFLWAWNAPSEFCLGKFDEPLDMSLFSFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDFLVETIKLGKLLRPNHLWGYYLFPDCYNHHYKKPGYNG
SCFNVEIKRNDDLSWLWNESTALYPSIYLNTQQSPVAATLYVRNRVREAIRVSKIPDAKSPLPVFAY
TRIVFTDQVLKFLSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDAFLKPPMETEEPQIF
SEQ ID NO: 847
LNFRAPPVIPNVPFLWAWNAPSEFCLGKFDEPLDMSLFSFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDFLVETIKLGKLLRPNHLWGYYLFPDCYNHHYKKPGYNG
SCFNVEIKRNDDLSWLWNESTALYPSIYLNTQQSPVAATLYVRNRVREAIRVSKIPDAKSPLPVFAY
TRIVFTDQVLKFLSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDAFLKPPMETEEPQI
SEQ ID NO: 848
LNFRAPPVIPNVPFLWAWNAPSEFCLGKFDEPLDMSLFSFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDFLVETIKLGKLLRPNHLWGYYLFPDCYNHHYKKPGYNG
SCFNVEIKRNDDLSWLWNESTALYPSIYLNTQQSPVAATLYVRNRVREAIRVSKIPDAKSPLPVFAY
TRIVFTDQVLKFLSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDAFLKPPMETEEPQ
SEQ ID NO: 849
LNFRAPPVIPNVPFLWAWNAPSEFCLGKFDEPLDMSLFSFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDFLVETIKLGKLLRPNHLWGYYLFPDCYNHHYKKPGYNG
SCFNVEIKRNDDLSWLWNESTALYPSIYLNTQQSPVAATLYVRNRVREAIRVSKIPDAKSPLPVFAY
TRIVFTDQVLKFLSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDAFLKPPMETEEP
SEQ ID NO: 850
LNFRAPPVIPNVPFLWAWNAPSEFCLGKFDEPLDMSLFSFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDFLVETIKLGKLLRPNHLWGYYLFPDCYNHHYKKPGYNG
SCFNVEIKRNDDLSWLWNESTALYPSIYLNTQQSPVAATLYVRNRVREAIRVSKIPDAKSPLPVFAY
TRIVFTDQVLKFLSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDAFLKPPMETEE
SEQ ID NO: 851
LNFRAPPVIPNVPFLWAWNAPSEFCLGKFDEPLDMSLFSFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDFLVETIKLGKLLRPNHLWGYYLFPDCYNHHYKKPGYNG
SCFNVEIKRNDDLSWLWNESTALYPSIYLNTQQSPVAATLYVRNRVREAIRVSKIPDAKSPLPVFAY
TRIVFTDQVLKFLSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDA
SEQ ID NO: 852
LNFRAPPVIPNVPFLWAWNAPSEFCLGKFDEPLDMSLFSFIGSPRINATGQGVTIFYVDRLGYYPYI
DSITGVTVNGGIPQKISLQDHLDKAKKDITFYMPVDNLGMAVIDWEEWRPTWARNWKPKDVYKNRSI
ELVQQQNVQLSLTEATEKAKQEFEKAGKDFLVETIKLGKLLRPNHLWGYYLFPDCYNHHYKKPGYNG
SCFNVEIKRNDDLSWLWNESTALYPSIYLNTQQSPVAATLYVRNRVREAIRVSKIPDAKSPLPVFAY
TRIVFTDQVLKFLSQDELVYTFGETVALGASGIVIWGTLSIMRSMKSCLLLDNYMETILNPYIINVT
LAAKMCSQVLCQEQGVCIRKNWNSSDYLHLNPDNFAIQLEKGGKFTVRGKPTLEDLEQFSEKFYCSC
YSTLSCKEKADVKDTDAVDVCIADGVCIDAFLKPPMETEEPQIFYN
SEQ ID NO: 853
atgggagtgc taaaattcaa gcacatcttt ttcagaagct ttgttaaatc aagtggagta tcccagatag ttttcacctt
ccttctgatt
ccatgttgct tgactctgaa tttcagagca cctcctgtta ttccaaatgt gcctttcctc tgggcctgga atgccccaag
tgaattttgt
cttggaaaat ttgatgagcc actagatatg agcctcttct ctttcatagg aagcccccga ataaacgcca ccgggcaagg
tgttacaata
ttttatgttg atagacttgg ctactatcct tacatagatt caatcacagg agtaactgtg aatggaggaa tcccccagaa
gatttcctta
caagaccatc tggacaaagc taagaaagac attacatttt atatgccagt agacaatttg ggaatggctg ttattgactg
ggaagaatgg
agacccactt gggcaagaaa ctggaaacct aaagatgttt acaagaatag gtctattgaa ttggttcagc aacaaaatgt
acaacttagt
ctcacagagg ccactgagaa agcaaaacaa gaatttgaaa aggcagggaa ggatttcctg gtagagacta taaaattggg
aaaattactt
cggccaaatc acttgtgggg ttattatctt tttccggatt gttacaacca tcactataag aaacccggtt acaatggaag
ttgcttcaat
gtagaaataa aaagaaatga tgatctcagc tggttgtgga atgaaagcac tgctctttac ccatccattt atttgaacac
tcagcagtct
cctgtagctg ctacactcta tgtgcgcaat cgagttcggg aagccatcag agtttccaaa atacctgatg caaaaagtcc
acttccggtt
tttgcatata cccgcatagt ttttactgat caagttttga aattcctttc tcaagatgaa cttgtgtata catttggcga
aactgttgct
ctgggtgctt ctggaattgt aatatgggga accctcagta taatgcgaag tatgaaatct tgcttgctcc tagacaatta
catggagact
atactgaatc cttacataat caacgtcaca ctagcagcca aaatgtgtag ccaagtgctt tgccaggagc aaggagtgtg
tataaggaaa
aactggaatt caagtgacta tcttcacctc aacccagata attttgctat tcaacttgag aaaggtggaa agttcacagt
acgtggaaaa
ccgacacttg aagacctgga gcaattttct gaaaaatttt attgcagctg ttatagcacc ttgagttgta aggagaaagc
tgatgtaaaa
gacactgatg ctgttgatgt gtgtattgct gatggtgtct gtatagatgc ttttctaaaa cctcccatgg agacagaaga
acctcaaatt
ttctac
SEQ ID NO: 854
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Leu Ser Ile Thr Arg Thr Lys Glu Ser Cys Gln Ala
Ile Lys Glu Tyr Met Asp Thr Thr Leu Gly Pro Tyr Ile Ile Asn Val Thr Leu Ala Ala Lys Met
Cys Ser Gln Val Leu Cys Gln Glu Gln Gly Val Cys Ile Arg Lys Asn Trp Asn Ser Ser Asp
Tyr Leu His Leu Asn Pro Asp Asn Phe Ala Ile Gln Leu Glu Lys Gly Gly Lys Phe Thr Val
Arg Gly Lys Pro Thr Leu Glu Asp Leu Glu Gln Phe Ser Glu Lys Phe Tyr Cys Ser Cys Tyr
Ser Thr Leu Ser Cys Lys Glu Lys Ala Asp Val Lys Asp Thr Asp Ala Val Asp Val Cys Ile
Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 855
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Leu Ser Ile Thr Arg Thr Lys Glu Ser Cys Gln Ala
Ile Lys Glu Tyr Met Asp Thr Thr Leu Asn Pro Phe Ile Leu Asn Val Thr Ser Gly Ala Leu
Leu Cys Ser Gln Ala Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 856
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Leu Ser Ile Thr Arg Thr Lys Glu Ser Cys Gln Ala
Ile Lys Glu Tyr Met Asp Thr Thr Leu Gly Pro Phe Ile Leu Asn Val Thr Ser Gly Ala Leu
Leu Cys Ser Gln Ala Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 857
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Val Ser Trp Glu Asn Thr Arg Thr Lys Glu Ser Cys Gln Ala
Ile Lys Glu Tyr Met Asp Thr Thr Leu Gly Pro Tyr Ile Ile Asn Val Thr Leu Ala Ala Lys Met
Cys Ser Gln Val Leu Cys Gln Glu Gln Gly Val Cys Ile Arg Lys Asn Trp Asn Ser Ser Asp
Tyr Leu His Leu Asn Pro Asp Asn Phe Ala Ile Gln Leu Glu Lys Gly Gly Lys Phe Thr Val
Arg Gly Lys Pro Thr Leu Glu Asp Leu Glu Gln Phe Ser Glu Lys Phe Tyr Cys Ser Cys Tyr
Ser Thr Leu Ser Cys Lys Glu Lys Ala Asp Val Lys Asp Thr Asp Ala Val Asp Val Cys Ile
Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 858
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Leu Ser Ile Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 859
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Val Ser Trp Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 860
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Leu Ser Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 861
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Leu Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 862
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Trp Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 863
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Leu Ser Ile Thr Arg Thr Lys Glu Ser Cys Gln Ala
Ile Lys Glu Tyr Met Asp Thr Thr Leu Asn Pro Phe Ile Leu Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 864
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Leu Ser Ile Thr Arg Thr Lys Glu Ser Cys Gln Ala
Ile Lys Glu Tyr Met Asp Thr Thr Leu Asn Pro Phe Ile Leu Asn Val Thr Ser Gly Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 865
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Leu Ser Ile Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys Pro Pro Met Glu Thr Glu Glu Pro Gln Ile Phe
Tyr Asn Ala Ser Pro Ser Thr Leu Ser
SEQ ID NO: 866
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Leu Ser Ile Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile
SEQ ID NO: 867
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Leu Ser Ile Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe
SEQ ID NO: 868
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu
Gly Met Ala Val Ile Asp Trp Glu Glu Trp Arg Pro Thr Trp Ala Arg Asn Trp Lys Pro Lys
Asp Val Tyr Lys Asn Arg Ser Ile Glu Leu Val Gln Gln Gln Asn Val Gln Leu Ser Leu Thr
Glu Ala Thr Glu Lys Ala Lys Gln Glu Phe Glu Lys Ala Gly Lys Asp Phe Leu Val Glu
Thr Ile Lys Leu Gly Lys Leu Leu Arg Pro Asn His Leu Trp Gly Tyr Tyr Leu Phe Pro Asp
Cys Tyr Asn His His Tyr Lys Lys Pro Gly Tyr Asn Gly Ser Cys Phe Asn Val Glu Ile Lys
Arg Asn Asp Asp Leu Ser Trp Leu Trp Asn Glu Ser Thr Ala Leu Tyr Pro Ser Ile Tyr Leu
Asn Thr Gln Gln Ser Pro Val Ala Ala Thr Leu Tyr Val Arg Asn Arg Val Arg Glu Ala Ile
Arg Val Ser Lys Ile Pro Asp Ala Lys Ser Pro Leu Pro Val Phe Ala Tyr Thr Arg Ile Val
Phe Thr Asp Gln Val Leu Lys Phe Leu Ser Gln Asp Glu Leu Val Tyr Thr Phe Gly Glu
Thr Val Ala Leu Gly Ala Ser Gly Ile Val Ile Trp Gly Thr Leu Ser Ile Thr Arg Thr Lys Glu
Ser Cys Gln Ala Ile Lys Glu Tyr Met Asp Thr Thr Leu Asn Pro Tyr Ile Ile Asn Val Thr
Leu Ala Ala Lys Met Cys Ser Gln Val Leu Cys Gln Glu Gln Gly Val Cys Ile Arg Lys Asn
Trp Asn Ser Ser Asp Tyr Leu His Leu Asn Pro Asp Asn Phe Ala Ile Gln Leu Glu Lys Gly
Gly Lys Phe Thr Val Arg Gly Lys Pro Thr Leu Glu Asp Leu Glu Gln Phe Ser Glu Lys
Phe Tyr Cys Ser Cys Tyr Ser Thr Leu Ser Cys Lys Glu Lys Ala Asp Val Lys Asp Thr Asp
Ala Val Asp Val Cys Ile Ala Asp Gly Val Cys Ile Asp Ala Phe Leu Lys Pro
SEQ ID NO: 869
Phe Arg Gly Pro Leu Leu Pro Asn Arg Pro Phe Leu Trp Ala Trp Asn Ala Pro Ser Glu Phe
Cys Leu Gly Lys Phe Asp Glu Pro Leu Asp Met Ser Leu Phe Ser Phe Ile Gly Ser Pro Arg
Ile Asn Ala Thr Gly Gln Gly Val Thr Ile Phe Tyr Val Asp Arg Leu Gly Tyr Tyr Pro Tyr Ile
Asp Ser Ile Thr Gly Val Thr Val Asn Gly Gly Ile Pro Gln Lys Ile Ser Leu Gln Asp His Leu
Asp Lys Ala Lys Lys Asp Ile Thr Phe Tyr Met Pro Val Asp Asn Leu Gly Met Ala Val Ile
Asp Trp Glu Glu Trp Arg Pro Thr Trp Ala Arg Asn Trp Lys Pro Lys Asp Val Tyr Lys Asn
Arg Ser Ile Glu Leu Val Gln Gln Gln Asn Val Gln Leu Ser Leu Thr Glu Ala Thr Glu Lys
Ala Lys Gln Glu Phe Glu Lys Ala Gly Lys Asp Phe Leu Val Glu Thr Ile Lys Leu Gly Lys
Leu Leu Arg Pro Asn His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg Asn Asp Asp Leu Ser
Trp Leu Trp Asn Glu Ser Thr Ala Leu Tyr Pro Ser Ile Tyr Leu Asn Thr Gln Gln Ser Pro
Val Ala Ala Thr Leu Tyr Val Arg Asn Arg Val Arg Glu Ala Ile Arg Val Ser Lys Ile Pro
Asp Ala Lys Ser Pro Leu Pro Val Phe Ala Tyr Thr Arg Ile Val Phe Thr Asp Gln Val Leu
Lys Phe Leu Ser Gln Asp Glu Leu Val Tyr Thr Phe Gly Glu Thr Val Ala Leu Gly Ala Ser
Gly Ile Val Ile Trp Gly Thr Leu Ser Ile Thr Arg Thr Lys Glu Ser Cys Gln Ala Ile Lys Glu
Tyr Met Asp Thr Thr Leu Asn Pro Tyr Ile Ile Asn Val Thr Leu Ala Ala Lys Met Cys Ser
Gln Val Leu Cys Gln Glu Gln Gly Val Cys Ile Arg Lys Asn Trp Asn Ser Ser Asp Tyr Leu
His Leu Asn Pro Asp Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys Ser Cys Tyr Ser Thr
Leu Ser Cys Lys Glu Lys Ala Asp Val Lys Asp Thr Asp Ala Val Asp Val Cys Ile Ala Asp
Gly Val Cys Ile Asp Ala Phe Leu Lys Pro Pro Met Glu Thr Glu Glu Pro Gln Ile Phe Tyr
Asn Ala Ser Pro Ser Thr Leu Ser
SEQ ID NO: 870
Phe Arg Gly Pro Leu Leu Pro Asn Arg Pro Phe Thr Thr Val Trp Asn Ala Pro Ser Glu Phe
Cys Leu Gly Lys Phe Asp Glu Pro Leu Asp Met Ser Leu Phe Ser Phe Ile Gly Ser Pro Arg
Ile Asn Ala Thr Gly Gln Gly Val Thr Ile Phe Tyr Val Asp Arg Leu Gly Tyr Tyr Pro Tyr Ile
Asp Ser Ile Thr Gly Val Thr Val Asn Gly Gly Ile Pro Gln Lys Ile Ser Leu Gln Asp His Leu
Asp Lys Ala Lys Lys Asp Ile Thr Phe Tyr Met Pro Val Asp Asn Leu Gly Met Ala Val Ile
Asp Trp Glu Glu Trp Arg Pro Thr Trp Ala Arg Asn Trp Lys Pro Lys Asp Val Tyr Lys Asn
Arg Ser Ile Glu Leu Val Gln Gln Gln Asn Val Gln Leu Ser Leu Thr Glu Ala Thr Glu Lys
Ala Lys Gln Glu Phe Glu Lys Ala Gly Lys Asp Phe Leu Val Glu Thr Ile Lys Leu Gly Lys
Leu Leu Arg Pro Asn His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg Asn Asp Asp Leu Ser
Trp Leu Trp Asn Glu Ser Thr Ala Leu Tyr Pro Ser Ile Tyr Leu Asn Thr Gln Gln Ser Pro
Val Ala Ala Thr Leu Tyr Val Arg Asn Arg Val Arg Glu Ala Ile Arg Val Ser Lys Ile Pro
Asp Ala Lys Ser Pro Leu Pro Val Phe Ala Tyr Thr Arg Ile Val Phe Thr Asp Gln Val Leu
Lys Phe Leu Ser Gln Asp Glu Leu Val Tyr Thr Phe Gly Glu Thr Val Ala Leu Gly Ala Ser
Gly Ile Val Ile Trp Gly Thr Leu Ser Ile Thr Arg Thr Lys Glu Ser Cys Gln Ala Ile Lys Glu
Tyr Met Asp Thr Thr Leu Asn Pro Tyr Ile Ile Asn Val Thr Leu Ala Ala Lys Met Cys Ser
Gln Val Leu Cys Gln Glu Gln Gly Val Cys Ile Arg Lys Asn Trp Asn Ser Ser Asp Tyr Leu
His Leu Asn Pro Asp Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys Ser Cys Tyr Ser Thr
Leu Ser Cys Lys Glu Lys Ala Asp Val Lys Asp Thr Asp Ala Val Asp Val Cys Ile Ala Asp
Gly Val Cys Ile Asp Ala Phe Leu Lys Pro Pro Met Glu Thr Glu Glu Pro Gln Ile Phe Tyr
Asn Ala Ser Pro Ser Thr Leu Ser
SEQ ID NO: 871
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys
SEQ ID NO: 872
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe
SEQ ID NO: 873
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Ser Trp Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 874
Ala Pro Pro Val Ile Pro Asn Val Pro Phe Leu Trp Ala Trp Asn Ala Pro Ser Glu Phe Cys
Leu Gly Lys Phe Asp Glu Pro Leu Asp Met Ser Leu Phe Ser Phe Ile Gly Ser Pro Arg Ile
Asn Ala Thr Gly Gln Gly Val Thr Ile Phe Tyr Val Asp Arg Leu Gly Tyr Tyr Pro Tyr Ile
Asp Ser Ile Thr Gly Val Thr Val Asn Gly Gly Ile Pro Gln Lys Ile Ser Leu Gln Asp His Leu
Asp Lys Ala Lys Lys Asp Ile Thr Phe Tyr Met Pro Val Asp Asn Leu Gly Met Ala Val Ile
Asp Trp Glu Glu Trp Arg Pro Thr Trp Ala Arg Asn Trp Lys Pro Lys Asp Val Tyr Lys Asn
Arg Ser Ile Glu Leu Val Gln Gln Gln Asn Val Gln Leu Ser Leu Thr Glu Ala Thr Glu Lys
Ala Lys Gln Glu Phe Glu Lys Ala Gly Lys Asp Phe Leu Val Glu Thr Ile Lys Leu Gly Lys
Leu Leu Arg Pro Asn His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg Asn Asp Asp Leu Ser
Trp Leu Trp Asn Glu Ser Thr Ala Leu Tyr Pro Ser Ile Tyr Leu Asn Thr Gln Gln Ser Pro
Val Ala Ala Thr Leu Tyr Val Arg Asn Arg Val Arg Glu Ala Ile Arg Val Ser Lys Ile Pro
Asp Ala Lys Ser Pro Leu Pro Val Phe Ala Tyr Thr Arg Ile Val Phe Thr Asp Gln Val Leu
Lys Phe Leu Ser Gln Asp Glu Leu Val Tyr Thr Phe Gly Glu Thr Val Ala Leu Gly Ala Ser
Gly Ile Val Ile Trp Gly Thr Leu Ser Ile Thr Arg Thr Lys Glu Ser Cys Gln Ala Ile Lys Glu
Tyr Met Asp Thr Thr Leu Asn Pro Tyr Ile Ile Asn Val Thr Leu Ala Ala Lys Met Cys Ser
Gln Val Leu Cys Gln Glu Gln Gly Val Cys Ile Arg Lys Asn Trp Asn Ser Ser Asp Tyr Leu
His Leu Asn Pro Asp Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys Ser Cys Tyr Ser Thr
Leu Ser Cys Lys Glu Lys Ala Asp Val Lys Asp Thr Asp Ala Val Asp Val Cys Ile Ala Asp
Gly Val Cys Ile Asp Ala Phe Leu Lys Pro Pro Met Glu Thr Glu Glu Pro Gln Ile Phe Tyr
Asn Ala Ser Pro Ser Thr Leu Ser
SEQ ID NO: 875
Pro Val Ile Pro Asn Val Pro Phe Leu Trp Ala Trp Asn Ala Pro Ser Glu Phe Cys Leu Gly
Lys Phe Asp Glu Pro Leu Asp Met Ser Leu Phe Ser Phe Ile Gly Ser Pro Arg Ile Asn Ala
Thr Gly Gln Gly Val Thr Ile Phe Tyr Val Asp Arg Leu Gly Tyr Tyr Pro Tyr Ile Asp Ser Ile
Thr Gly Val Thr Val Asn Gly Gly Ile Pro Gln Lys Ile Ser Leu Gln Asp His Leu Asp Lys
Ala Lys Lys Asp Ile Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val Tyr Lys Asn Arg Ser
Ile Glu Leu Val Gln Gln Gln Asn Val Gln Leu Ser Leu Thr Glu Ala Thr Glu Lys Ala Lys
Gln Glu Phe Glu Lys Ala Gly Lys Asp Phe Leu Val Glu Thr Ile Lys Leu Gly Lys Leu Leu
Arg Pro Asn His Leu Trp Gly Tyr Tyr Leu Phe Pro Asp Cys Tyr Asn His His Tyr Lys Lys
Pro Gly Tyr Asn Gly Ser Cys Phe Asn Val Glu Ile Lys Arg Asn Asp Asp Leu Ser Trp Leu
Trp Asn Glu Ser Thr Ala Leu Tyr Pro Ser Ile Tyr Leu Asn Thr Gln Gln Ser Pro Val Ala Ala
Thr Leu Tyr Val Arg Asn Arg Val Arg Glu Ala Ile Arg Val Ser Lys Ile Pro Asp Ala Lys
Ser Pro Leu Pro Val Phe Ala Tyr Thr Arg Ile Val Phe Thr Asp Gln Val Leu Lys Phe Leu
Ser Gln Asp Glu Leu Val Tyr Thr Phe Gly Glu Thr Val Ala Leu Gly Ala Ser Gly Ile Val Ile
Trp Gly Thr Leu Ser Ile Thr Arg Thr Lys Glu Ser Cys Gln Ala Ile Lys Glu Tyr Met Asp
Thr Thr Leu Asn Pro Tyr Ile Ile Asn Val Thr Leu Ala Ala Lys Met Cys Ser Gln Val Leu
Cys Gln Glu Gln Gly Val Cys Ile Arg Lys Asn Trp Asn Ser Ser Asp Tyr Leu His Leu Asn
Pro Asp Asn Phe Ala Ile Gln Leu Glu Lys Gly Gly Lys Phe Thr Val Arg Gly Lys Pro Thr
Leu Glu Asp Leu Glu Gln Phe Ser Glu Lys Phe Tyr Cys Ser Cys Tyr Ser Thr Leu Ser Cys
Lys Glu Lys Ala Asp Val Lys Asp Thr Asp Ala Val Asp Val Cys Ile Ala Asp Gly Val Cys
Ile Asp Ala Phe Leu Lys Pro Pro Met Glu Thr Glu Glu Pro Gln Ile Phe Tyr Asn Ala Ser Pro
Ser Thr Leu Ser
SEQ ID NO: 876
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Trp Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala
SEQ ID NO: 877
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Trp Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys
SEQ ID NO: 878
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Trp Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Pro Thr Leu Glu Asp Leu Glu Gln Phe Ser Glu Lys Phe Tyr Cys Ser Cys Tyr
Ser Thr Leu Ser Cys Lys Glu Lys Ala Asp Val Lys Asp Thr Asp Ala Val Asp Val Cys Ile
Ala Asp
SEQ ID NO: 879
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Trp Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
SEQ ID NO: 880
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Thr Trp Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val
SEQ ID NO: 881
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Ser Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys
SEQ ID NO: 882
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys Pro Pro
SEQ ID NO: 883
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met
Ala Val Ile Asp Trp Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu Ser Leu Thr Glu Ala
Thr Glu Lys Ala Lys Gln Glu Phe Glu Lys Ala Gly Lys Asp Phe Leu Val Glu Thr Ile Lys
Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe Gly Glu Thr Val
Ala Leu Gly Ala Ser Gly Ile Val Ile Trp Gly Thr Trp Glu Asn Thr Arg Thr Lys Glu Ser
Cys Gln Ala Ile Lys Glu Tyr Met Asp Thr Thr Leu Asn Pro Tyr Ile Ile Asn Val Thr Leu
Ala Ala Lys Met Cys Ser Gln Val Leu Cys Gln Glu Gln Gly Val Cys Ile Arg Lys Asn Trp
Asn Ser Ser Asp Tyr Leu His Leu Asn Pro Asp Asn Phe Ala Ile Gln Leu Glu Lys Gly Gly
Lys Phe Thr Val Arg Gly Lys Pro Thr Leu Glu Asp Leu Glu Gln Phe Ser Glu Lys Phe Tyr
Cys Ser Cys Tyr Ser Thr Leu Ser Cys Lys Glu Lys Ala Asp Val Lys Asp Thr Asp Ala Val
Asp Val Cys Ile Ala Asp Gly Val Cys Ile Asp Ala Phe Leu Lys Pro Pro Met
SEQ ID NO: 884
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys Pro Pro Met Glu
SEQ ID NO: 885
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys Pro Pro Met Glu Thr
SEQ ID NO: 886
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys Pro Pro Met Glu Thr Glu
SEQ ID NO: 887
Asn Phe Arg Ala Pro Pro Val Ile Pro Asn Val Pro Phe Leu Trp Ala Trp Asn Ala Pro Ser
Glu Phe Cys Leu Gly Lys Phe Asp Glu Pro Leu Asp Met Ser Leu Phe Ser Phe Ile Gly Ser
Pro Arg Ile Asn Ala Thr Gly Gln Gly Val Thr Ile Phe Tyr Val Asp Arg Leu Gly Tyr Tyr
Pro Tyr Ile Asp Ser Ile Thr Gly Val Thr Val Asn Gly Gly Ile Pro Gln Lys Ile Ser Leu Gln
Asp His Leu Asp Lys Ala Lys Lys Asp Ile Thr Phe Tyr Met Pro Val Asp Asn Leu Gly Met
Ala Val Ile Asp Trp Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu Ser Leu Thr Glu Ala
Thr Glu Lys Ala Lys Gln Glu Phe Glu Lys Ala Gly Lys Asp Phe Leu Val Glu Thr Ile Lys
Leu Gly Lys Leu Leu Arg Pro Asn His Leu Trp Gly Tyr Tyr Leu Phe Pro Asp Cys Tyr Asn
His His Tyr Lys Lys Pro Gly Tyr Asn Gly Ser Cys Phe Asn Val Glu Ile Lys Arg Asn Asp
Asp Leu Ser Trp Leu Trp Asn Glu Ser Thr Ala Leu Tyr Pro Ser Ile Tyr Leu Asn Thr Gln
Gln Ser Pro Val Ala Ala Thr Leu Tyr Val Arg Asn Arg Val Arg Glu Ala Ile Arg Val Ser
Lys Ile Pro Asp Ala Lys Ser Pro Leu Pro Val Phe Ala Tyr Thr Arg Ile Val Phe Thr Asp Gln
Val Leu Lys Phe Leu Ser Gln Asp Glu Leu Val Tyr Thr Phe Gly Glu Thr Val Ala Leu Gly
Ala Ser Gly Ile Val Ile Trp Gly Thr Leu Ser Ile Thr Arg Thr Lys Glu Ser Cys Gln Ala Ile
Lys Glu Tyr Met Asp Thr Thr Leu Asn Pro Tyr Ile Ile Asn Val Thr Leu Ala Ala Lys Met
Cys Ser Gln Val Leu Cys Gln Glu Gln Gly Val Cys Ile Arg Lys Asn Trp Asn Ser Ser Asp
Tyr Leu His Leu Asn Pro Asp Asn Phe Ala Ile Gln Leu Glu Lys Gly Gly Lys Phe Thr Val
Arg Gly Lys Pro Thr Leu Glu Asp Leu Glu Gln Phe Ser Glu Lys Phe Tyr Cys Ser Cys Tyr
Ser Thr Leu Ser Cys Lys Glu Lys Ala Asp Val Lys Asp Thr Asp Ala Val Asp Val Cys Ile
Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 888
Arg Ala Pro Pro Val Ile Pro Asn Val Pro Phe Leu Trp Ala Trp Asn Ala Pro Ser Glu Phe
Cys Leu Gly Lys Phe Asp Glu Pro Leu Asp Met Ser Leu Phe Ser Phe Ile Gly Ser Pro Arg
Ile Asn Ala Thr Gly Gln Gly Val Thr Ile Phe Tyr Val Asp Arg Leu Gly Tyr Tyr Pro Tyr Ile
Asp Ser Ile Thr Gly Val Thr Val Asn Gly Gly Ile Pro Gln Lys Ile Ser Leu Gln Asp His Leu
Asp Lys Ala Lys Lys Asp Ile Thr Phe Tyr Met Pro Val Asp Asn Leu Gly Met Ala Val Ile
Asp Trp Glu Glu Trp Arg Pro Thr Trp Ala Arg Asn Trp Lys Pro Lys Asp Val Tyr Lys Asn
Arg Ser Ile Glu Leu Val Gln Gln Gln Asn Val Gln Leu Ser Leu Thr Glu Ala Thr Glu Lys
Ala Lys Gln Glu Phe Glu Lys Ala Gly Lys Asp Phe Leu Val Glu Thr Ile Lys Leu Gly Lys
Leu Leu Arg Pro Asn His Leu Trp Gly Tyr Tyr Leu Phe Pro Asp Cys Tyr Asn His His Tyr
Lys Lys Pro Gly Tyr Asn Gly Ser Cys Phe Asn Val Glu Ile Lys Arg Asn Asp Asp Leu Ser
Trp Leu Trp Asn Glu Ser Thr Ala Leu Tyr Pro Ser Ile Tyr Leu Asn Thr Gln Gln Ser Pro
Val Ala Ala Thr Leu Tyr Val Arg Asn Arg Val Arg Glu Ala Ile Arg Val Ser Lys Ile Pro
Asp Ala Lys Ser Pro Leu Pro Val Phe Ala Tyr Thr Arg Ile Val Phe Thr Asp Gln Val Leu
Lys Phe Leu Ser Gln Asp Glu Leu Val Tyr Thr Phe Gly Glu Thr Val Ala Leu Gly Ala Ser
Gly Ile Val Ile Trp Gly Thr Leu Ser Ile Thr Arg Thr Lys Glu Ser Cys Gln Ala Ile Lys Glu
Tyr Met Asp Thr Thr Leu Asn Pro Tyr Ile Ile Asn Val Thr Leu Ala Ala Lys Met Cys Ser
Gln Val Leu Cys Gln Glu Gln Gly Val Cys Ile Arg Lys Asn Trp Asn Ser Ser Asp Tyr Leu
His Leu Asn Pro Asp Asn Phe Ala Ile Gln Leu Glu Lys Gly Gly Lys Phe Thr Val Arg Gly
Lys Pro Thr Leu Glu Asp Leu Glu Gln Phe Ser Glu Lys Phe Tyr Cys Ser Cys Tyr Ser Thr
Leu Ser Cys Lys Glu Lys Ala Asp Val Lys Asp Thr Asp Ala Val Asp Val Cys Ile Ala Asp
Gly Val Cys Ile Asp Ala Phe Leu Lys Pro Pro Met Glu Thr Glu Glu Pro Gln Ile Phe Tyr
Asn Ala Ser Pro Ser Thr Leu Ser
SEQ ID NO: 889
Pro Pro Val Ile Pro Asn Val Pro Phe Leu Trp Ala Trp Asn Ala Pro Ser Glu Phe Cys Leu
Gly Lys Phe Asp Glu Pro Leu Asp Met Ser Leu Phe Ser Phe Ile Gly Ser Pro Arg Ile Asn
Ala Thr Gly Gln Gly Val Thr Ile Phe Tyr Val Asp Arg Leu Gly Tyr Tyr Pro Tyr Ile Asp Ser
Ile Thr Gly Val Thr Val Asn Gly Gly Ile Pro Gln Lys Ile Ser Leu Gln Asp His Leu Asp Lys
Ala Lys Lys Asp Ile Thr Phe Tyr Met Pro Val Asp Asn Leu Gly Met Ala Val Ile Asp Trp
Glu Glu Trp Arg Pro Thr Trp Ala Arg Asn Trp Lys Pro Lys Asp Val Tyr Lys Asn Arg Ser
Ile Glu Leu Val Gln Gln Gln Asn Val Gln Leu Ser Leu Thr Glu Ala Thr Glu Lys Ala Lys
Gln Glu Phe Glu Lys Ala Gly Lys Asp Phe Leu Val Glu Thr Ile Lys Leu Gly Lys Leu Leu
Arg Pro Asn His Leu Trp Gly Tyr Tyr Leu Phe Pro Asp Cys Tyr Asn His His Tyr Lys Lys
Pro Gly Tyr Asn Gly Ser Cys Phe Asn Val Glu Ile Lys Arg Asn Asp Asp Leu Ser Trp Leu
Trp Asn Glu Ser Thr Ala Leu Tyr Pro Ser Ile Tyr Leu Asn Thr Gln Gln Ser Pro Val Ala Ala
Thr Leu Tyr Val Arg Asn Arg Val Arg Glu Ala Ile Arg Val Ser Lys Ile Pro Asp Ala Lys
Ser Pro Leu Pro Val Phe Ala Tyr Thr Arg Ile Val Phe Thr Asp Gln Val Leu Lys Phe Leu
Ser Gln Asp Glu Leu Val Tyr Thr Phe Gly Glu Thr Val Ala Leu Gly Ala Ser Gly Ile Val Ile
Trp Gly Thr Leu Ser Ile Thr Arg Thr Lys Glu Ser Cys Gln Ala Ile Lys Glu Tyr Met Asp
Thr Thr Leu Asn Pro Tyr Ile Ile Asn Val Thr Leu Ala Ala Lys Met Cys Ser Gln Val Leu
Cys Gln Glu Gln Gly Val Cys Ile Arg Lys Asn Trp Asn Ser Ser Asp Tyr Leu His Leu Asn
Pro Asp Asn Phe Ala Ile Gln Leu Glu Lys Gly Gly Lys Phe Thr Val Arg Gly Lys Pro Thr
Leu Glu Asp Leu Glu Gln Phe Ser Glu Lys Phe Tyr Cys Ser Cys Tyr Ser Thr Leu Ser Cys
Lys Glu Lys Ala Asp Val Lys Asp Thr Asp Ala Val Asp Val Cys Ile Ala Asp Gly Val Cys
Ile Asp Ala Phe Leu Lys Pro Pro Met Glu Thr Glu Glu Pro Gln Ile Phe Tyr Asn Ala Ser Pro
Ser Thr Leu Ser
SEQ ID NO: 890
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Ser Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile
SEQ ID NO: 891
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Ser Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp
SEQ ID NO: 892
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Ser Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala
SEQ ID NO: 893
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Ser Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe
SEQ ID NO: 894
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys Pro Pro Met Glu Thr Glu Glu Pro
SEQ ID NO: 895
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys Pro Pro Met Glu Thr Glu Glu Pro Gln Ile
SEQ ID NO: 896
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys Pro Pro Met Glu Thr Glu Glu Pro Gln Ile Phe
Tyr
SEQ ID NO: 897
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys Pro Pro Met Glu Thr Glu Glu Pro Gln Ile Phe
Tyr Asn Ala
SEQ ID NO: 898
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys Pro Pro Met Glu Thr Glu Glu Pro Gln Ile Phe
Tyr Asn Ala Ser Pro
SEQ ID NO: 899
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe Leu Lys Pro Pro Met Glu Thr Glu Glu Pro Gln Ile Phe
Tyr Asn Ala Ser Pro Ser Thr
SEQ ID NO: 900
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Gly Trp Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 901
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Ala Trp Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 902
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Cys Trp Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 903
Leu Asn Phe Arg Ala Pro Pro Val Ile Pro Asn Val Pro Phe Leu Trp Ala Trp Asn Ala Pro
Ser Glu Phe Cys Leu Gly Lys Phe Asp Glu Pro Leu Asp Met Ser Leu Phe Ser Phe Ile Gly
Ser Pro Arg Ile Asn Ala Thr Gly Gln Gly Val Thr Ile Phe Tyr Val Asp Arg Leu Gly Tyr
Tyr Pro Tyr Ile Asp Ser Ile Thr Gly Val Thr Val Asn Gly Gly Ile Pro Gln Lys Ile Ser Leu
Gln Asp His Leu Asp Lys Ala Lys Lys Asp Ile Thr Phe Tyr Met Pro Val Asp Asn Leu Gly
Met Ala Val Ile Asp Trp Glu Glu Trp Arg Pro Thr Trp Ala Arg Asn Trp Lys Pro Lys Asp
Val Tyr Lys Asn Arg Ser Ile Glu Leu Val Gln Gln Gln Asn Val Gln Leu Ser Leu Thr Glu
Ala Thr Glu Lys Ala Lys Gln Glu Phe Glu Lys Ala Gly Lys Asp Phe Leu Val Glu Thr Ile
Lys Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr Asp
Gln Val Leu Lys Phe Leu Ser Gln Asp Glu Leu Val Tyr Thr Phe Gly Glu Thr Val Ala Leu
Gly Ala Ser Gly Ile Val Ile Trp Gly Asp Trp Glu Asn Thr Arg Thr Lys Glu Ser Cys Gln Ala
Ile Lys Glu Tyr Met Asp Thr Thr Leu Asn Pro Tyr Ile Ile Asn Val Thr Leu Ala Ala Lys
Met Cys Ser Gln Val Leu Cys Gln Glu Gln Gly Val Cys Ile Arg Lys Asn Trp Asn Ser Ser
Asp Tyr Leu His Leu Asn Pro Asp Asn Phe Ala Ile Gln Leu Glu Lys Gly Gly Lys Phe Thr
Val Arg Gly Lys Pro Thr Leu Glu Asp Leu Glu Gln Phe Ser Glu Lys Phe Tyr Cys Ser Cys
Tyr Ser Thr Leu Ser Cys Lys Glu Lys Ala Asp Val Lys Asp Thr Asp Ala Val Asp Val Cys
Ile Ala Asp Gly Val Cys Ile Asp Ala Phe Leu Lys Pro Pro Met Glu Thr Glu Glu Pro Gln Ile
Phe Tyr Asn Ala Ser Pro Ser Thr Leu Ser
SEQ ID NO: 904
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Leu Ser Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe
SEQ ID NO: 905
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Thr Leu Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe
SEQ ID NO: 906
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Ala Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe
SEQ ID NO: 907
Phe Arg Ala Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met
Ala Val Ile Asp Trp Glu Glu Trp Arg Pro Thr Trp Ala Arg Asn Trp Lys Pro Lys Asp Val
Tyr Lys Asn Arg Ser Ile Glu Leu Val Gln Gln Gln Asn Val Gln Leu Ser Leu Thr Glu Ala
Thr Glu Lys Ala Lys Gln Glu Phe Glu Lys Ala Gly Lys Asp Phe Leu Val Glu Thr Ile Lys
Leu Gly Lys Leu Leu Arg Pro Asn His Leu Trp Gly Tyr Tyr Leu Phe Pro Asp Cys Tyr
Asn His His Tyr Lys Lys Pro Gly Tyr Asn Gly Ser Cys Phe Asn Val Glu Ile Lys Arg Asn
Asp Asp Leu Ser Trp Leu Trp Asn Glu Ser Thr Ala Leu Tyr Pro Ser Ile Tyr Leu Asn Thr
Gln Gln Ser Pro Val Ala Ala Thr Leu Tyr Val Arg Asn Arg Val Arg Glu Ala Ile Arg Val
Ser Lys Ile Pro Asp Ala Lys Ser Pro Leu Pro Val Phe Ala Tyr Thr Arg Ile Val Phe Thr
Asp Gln Val Leu Lys Phe Leu Ser Gln Asp Glu Leu Val Tyr Thr Phe Gly Glu Thr Val
Ala Leu Gly Ala Ser Gly Ile Val Ile Trp Gly Gly Trp Glu Asn Thr Arg Thr Lys Glu Ser
Cys Gln Ala Ile Lys Glu Tyr Met Asp Thr Thr Leu Asn Pro Tyr Ile Ile Asn Val Thr Leu
Ala Ala Lys Met Cys Ser Gln Val Leu Cys Gln Glu Gln Gly Val Cys Ile Arg Lys Asn Trp
Asn Ser Ser Asp Tyr Leu His Leu Asn Pro Asp Asn Phe Ala Ile Gln Leu Glu Lys Gly Gly
Lys Phe Thr Val Arg Gly Lys Pro Thr Leu Glu Asp Leu Glu Gln Phe Ser Glu Lys Phe Tyr
Cys Ser Cys Tyr Ser Thr Leu Ser Cys Lys Glu Lys Ala Asp Val Lys Asp Thr Asp Ala Val
Asp Val Cys Ile Ala Asp Gly Val Cys Ile Asp Ala Phe
SEQ ID NO: 908
Phe Arg Gly Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Ser Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe
SEQ ID NO: 909
Phe Arg Gly Pro Pro Val Ile Pro Asn Val Pro Phe Leu Trp Ala Trp Asn Ala Pro Ser Glu
Phe Cys Leu Gly Lys Phe Asp Glu Pro Leu Asp Met Ser Leu Phe Ser Phe Ile Gly Ser Pro
Arg Ile Asn Ala Thr Gly Gln Gly Val Thr Ile Phe Tyr Val Asp Arg Leu Gly Tyr Tyr Pro
Tyr Ile Asp Ser Ile Thr Gly Val Thr Val Asn Gly Gly Ile Pro Gln Lys Ile Ser Leu Gln Asp
His Leu Asp Lys Ala Lys Lys Asp Ile Thr Phe Tyr Met Pro Val Asp Asn Leu Gly Met Ala
Val Ile Asp Trp Glu Glu Trp Arg Pro Thr Trp Ala Arg Asn Trp Lys Pro Lys Asp Val Tyr
Lys Asn Arg Ser Ile Glu Leu Val Gln Gln Gln Asn Val Gln Leu Ser Leu Thr Glu Ala Thr
Glu Lys Ala Lys Gln Glu Phe Glu Lys Ala Gly Lys Asp Phe Leu Val Glu Thr Ile Lys Leu
Gly Lys Leu Leu Arg Pro Asn His Leu Trp Gly Tyr Tyr Leu Phe Pro Asp Cys Tyr Asn His
His Tyr Lys Lys Pro Gly Tyr Asn Gly Ser Cys Phe Asn Val Glu Ile Lys Arg Asn Asp Asp
Leu Ser Trp Leu Trp Asn Glu Ser Thr Ala Leu Tyr Pro Ser Ile Tyr Leu Asn Thr Gln Gln
Ser Pro Val Ala Ala Thr Leu Tyr Val Arg Asn Arg Val Arg Glu Ala Ile Arg Val Ser Lys Ile
Pro Asp Ala Lys Ser Pro Leu Pro Val Phe Ala Tyr Thr Arg Ile Val Phe Thr Asp Gln Val
Leu Lys Phe Leu Ser Gln Asp Glu Leu Val Tyr Thr Phe Gly Glu Thr Val Ala Leu Gly Ala
Ser Gly Ile Val Ile Trp Gly Ser Trp Glu Asn Thr Arg Thr Lys Glu Ser Cys Gln Ala Ile Lys
Glu Tyr Met Asp Thr Thr Leu Asn Pro Tyr Ile Ile Asn Val Thr Leu Ala Ala Lys Met Cys
Ser Gln Val Leu Cys Gln Glu Gln Gly Val Cys Ile Arg Lys Asn Trp Asn Ser Ser Asp Tyr
Leu His Leu Asn Pro Asp Asn Phe Ala Ile Gln Leu Glu Lys Gly Gly Lys Phe Thr Val Arg
Gly Lys Pro Thr Leu Glu Asp Leu Glu Gln Phe Ser Glu Lys Phe Tyr Cys Ser Cys Tyr Ser
Thr Leu Ser Cys Lys Glu Lys Ala Asp Val Lys Asp Thr Asp Ala Val Asp Val Cys Ile Ala
Asp Gly Val Cys Ile Asp Ala Phe

SEQ ID NO: 910
ISGSGGAT
SEQ ID NO: 911
TKDRLIMATVRGPYYYGMDV
SEQ ID NO: 912
QQSHSVSFT

| | | SEQ ID |
|---|---|---|
| Description | Sequence | NO |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 913 |
| hIgG1* C- | NHKPSNTKVDKKVEPKSCDKTHTCPPCPAPELLGGPSVFLFP | |
| terminal lysine | PKPKDTLMISRTPEVTCVVVDVSHEDPEVKFNWYVDGVEVH | |
| variant | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 914 |
| hIgG4 C- | DHKPSNTKVDKRVESKYGPPCPSCPAPEFLGGPSVFLFPPKPK | |
| terminal lysine | DTLMISRTPEVTCVVVDVSQEDPEVQFNWYVDGVEVHNAK | |
| variant | TKPREEQFNSTYRVVSVLTVLHQDWLNGKEYKCKVSNKGLP | |
| | SSIEKTISKAKGQPREPQVYTLPPSQEEMTKNQVSLTCLVKGF | |
| | YPSDIAVEWESNGQPENNYKTTPPVLDSDGSFFLYSRLTVDK | |
| | SRWQEGNVFSCSVMHEALHNHYTQKSLSLSLGK | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 915 |
| IgG4-SP C- | DHKPSNTKVDKRVESKYGPPCPPCPAPEFLGGPSVFLFPPKPK | |
| terminal lysine | DTLMISRTPEVTCVVVDVSQEDPEVQFNWYVDGVEVHNAK | |
| variant | TKPREEQFNSTYRVVSVLTVLHQDWLNGKEYKCKVSNKGLP | |
| | SSIEKTISKAKGQPREPQVYTLPPSQEEMTKNQVSLTCLVKGF | |
| | YPSDIAVEWESNGQPENNYKTTPPVLDSDGSFFLYSRLTVDK | |
| | SRWQEGNVFSCSVMHEALHNHYTQKSLSLSLGK | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 916 |
| IgG4-SPLE C- | DHKPSNTKVDKRVESKYGPPCPPCPAPEELGGPSVFLFPPKPK | |
| terminal lysine | DTLMISRTPEVTCVVVDVSQEDPEVQFNWYVDGVEVHNAK | |
| variant | TKPREEQFNSTYRVVSVLTVLHQDWLNGKEYKCKVSNKGLP | |
| | SSIEKTISKAKGQPREPQVYTLPPSQEEMTKNQVSLTCLVKGF | |
| | YPSDIAVEWESNGQPENNYKTTPPVLDSDGSFFLYSRLTVDK | |
| | SRWQEGNVFSCSVMHEALHNHYTQKSLSLSLGK | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSNFGTQTYTCNV | NO: 917 |
| IgG2 C-terminal | DHKPSNTKVDKTVERKCCVECPPCPAPPVAGPSVFLFPPKPK | |
| lysine variant | DTLMISRTPEVTCVVVDVSHEDPEVQFNWYVDGVEVHNAK | |
| | TKPREEQFNSTFRVVSVLTVVHQDWLNGKEYKCKVSNKGLP | |
| | APIEKTISKTKGQPREPQVYTLPPSREEMTKNQVSLTCLVKGF | |
| | YPSDIAVEWESNGQPENNYKTTPPMLDSDGSFFLYSKLTVDK | |
| | SRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 918 |
| hIgG1-N297A | NHKPSNTKVDKKVEPKSCDKTHTCPPCPAPELLGGPSVFLFP | |
| C-terminal lysine | PKPKDTLMISRTPEVTCVVVDVSHEDPEVKFNWYVDGVEVH | |
| variant | NAKTKPREEQYASTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 919 |
| hIgG1-D265A | NHKPSNTKVDKKVEPKSCDKTHTCPPCPAPELLGGPSVFLFP | |
| C-terminal lysine | PKPKDTLMISRTPEVTCVVVAVSHEDPEVKFNWYVDGVEVH | |
| variant | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 920 |
| hIgG1-LALA C- | NHKPSNTKVDKKVEPKSCDKTHTCPPCPAPEAAGGPSVFLFP | |
| terminal lysine | PKPKDTLMISRTPEVTCVVVDVSHEDPEVKFNWYVDGVEVH | |
| variant | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 921 |
| hIgG1-LAGA C- | NHKPSNTKVDKKVEPKSCDKTHTCPPCPAPELAGAPSVFLFP | |
| terminal lysine | PKPKDTLMISRTPEVTCVVVDVSHEDPEVKFNWYVDGVEVH | |
| variant | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 922 |
| hIgG1- | NHKPSNTKVDKKVEPKSCDKTHTCPPCPAPEAAGAPSVFLFP | |
| LALAGA C- | PKPKDTLMISRTPEVTCVVVDVSHEDPEVKFNWYVDGVEVH | |
| terminal lysine | NAKTKPREEQYNSTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 923 |
| hIgG1-LALAPG | NHKPSNTKVDKKVEPKSCDKTHTCPPCPAPEAAGGPSVFLFP | |
| C-terminal lysine | PKPKDTLMISRTPEVTCVVVDVSHEDPEVKFNWYVDGVEVH | |
| variant | NAKTKPREEQYNSTYRVVSVLTVLHQDWLNGKEYKCKVSN | |
| | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 924 |
| hIgG1-YTE C- | NHKPSNTKVDKKVEPKSCDKTHTCPPCPAPELLGGPSVFLFP | |
| terminal lysine | PKPKDTLYITREPEVTCVVVDVSHEDPEVKFNWYVDGVEVH | |
| variant | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 925 |
| hIgG1- | NHKPSNTKVDKKVEPKSCDKTHTCPPCPAPELLGGPSVFLFP | |
| N297A/YTE C- | PKPKDTLYITREPEVTCVVVDVSHEDPEVKFNWYVDGVEVH | |
| terminal lysine | NAKTKPREEQYASTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 926 |
| hIgG1- | NHKPSNTKVDKKVEPKSCDKTHTCPPCPAPELLGGPSVFLFP | |
| D265A/YTE C- | PKPKDTLYITREPEVTCVVVAVSHEDPEVKFNWYVDGVEVH | |
| terminal lysine | NAKTKPREEQYNSTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 927 |
| hIgG1- | NHKPSNTKVDKKVEPKSCDKTHTCPPCPAPEAAGGPSVFLFP | |
| LALA/YTE C- | PKPKDTLYITREPEVTCVVVDVSHEDPEVKFNWYVDGVEVH | |
| terminal lysine | NAKTKPREEQYNSTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 928 |
| hIgG1- | NHKPSNTKVDKKVEPKSCDKTHTCPPCPAPELAGAPSVFLFP | |
| LAGA/YTE C- | PKPKDTLYITREPEVTCVVVDVSHEDPEVKFNWYVDGVEVH | |
| terminal lysine | NAKTKPREEQYNSTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 929 |
| hIgG1- | NHKPSNTKVDKKVEPKSCDKTHTCPPCPAPEAAGAPSVFLFP | |
| LALAGA/YTE | PKPKDTLYITREPEVTCVVVDVSHEDPEVKFNWYVDGVEVH | |
| C-terminal lysine | NAKTKPREEQYNSTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 930 |
| hIgG1- | NHKPSNTKVDKKVEPKSCDKTHTCPPCPAPEAAGGPSVFLFP | |
| LALAPG/YTE | PKPKDTLYITREPEVTCVVVDVSHEDPEVKFNWYVDGVEVH | |
| C-terminal lysine | NAKTKPREEQYNSTYRVVSVLTVLHQDWLNGKEYKCKVSN | |
| variant | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 931 |
| hIgG1-LS C- | NHKPSNTKVDKKVEPKSCDKTHTCPPCPAPELLGGPSVFLFP | |
| terminal lysine | PKPKDTLMISRTPEVTCVVVDVSHEDPEVKFNWYVDGVEVH | |
| variant | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHSHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 932 |
| hIgG1- | NHKPSNTKVDKKVEPKSCDKTHTCPPCPAPELLGGPSVFLFP | |
| N297A/LS C- | PKPKDTLMISRTPEVTCVVVDVSHEDPEVKFNWYVDGVEVH | |
| terminal lysine | NAKTKPREEQYASTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHSHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 933 |
| hIgG1- | NHKPSNTKVDKKVEPKSCDKTHTCPPCPAPELLGGPSVFLFP | |
| D265A/LS C- | PKPKDTLMISRTPEVTCVVVAVSHEDPEVKFNWYVDGVEVH | |
| terminal lysine | NAKTKPREEQYNSTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHSHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 934 |
| hIgG1- | NHKPSNTKVDKKVEPKSCDKTHTCPPCPAPEAAGGPSVFLFP | |
| LALA/LS C- | PKPKDTLMISRTPEVTCVVVDVSHEDPEVKFNWYVDGVEVH | |
| terminal lysine | NAKTKPREEQYNSTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHSHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 935 |
| hIgG1- | NHKPSNTKVDKKVEPKSCDKTHTCPPCPAPELAGAPSVFLFP | |
| LAGA/LS C- | PKPKDTLMISRTPEVTCVVVDVSHEDPEVKFNWYVDGVEVH | |
| terminal lysine | NAKTKPREEQYNSTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHSHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 936 |
| hIgG1- | NHKPSNTKVDKKVEPKSCDKTHTCPPCPAPEAAGAPSVFLFP | |
| LALAGA/LS C- | PKPKDTLMISRTPEVTCVVVDVSHEDPEVKFNWYVDGVEVH | |
| terminal lysine | NAKTKPREEQYNSTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHSHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 937 |
| hIgG1- | NHKPSNTKVDKKVEPKSCDKTHTCPPCPAPEAAGGPSVFLFP | |
| LALAPG/LS C- | PKPKDTLMISRTPEVTCVVVDVSHEDPEVKFNWYVDGVEVH | |
| terminal lysine | NAKTKPREEQYNSTYRVVSVLTVLHQDWLNGKEYKCKVSN | |
| variant | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHSHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 938 |
| hIgG1-DHS C- | NHKPSNTKVDKKVEPKSCDKTHTCPPCPAPELLGGPSVFLFP | |
| terminal lysine | PKPKDTLMISRTPEVTCVVVDVSHEDPEVKFNWYVDGVEVH | |
| variant | NAKTKPREEQYNSTYRVVSVLTVDHHDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHSHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | |
| hIgG1- | NHKPSNTKVDKKVEPKSCDKTHTCPPCPAPELLGGPSVFLFP | |
| N297A/DHS C- | PKPKDTLMISRTPEVTCVVVDVSHEDPEVKFNWYVDGVEVH | NO: 939 |
| terminal lysine | NAKTKPREEQYASTYRVVSVLTVDHHDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHSHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 940 |
| hIgG1- | NHKPSNTKVDKKVEPKSCDKTHTCPPCPAPELLGGPSVFLFP | |
| D265A/DHS C- | PKPKDTLMISRTPEVTCVVVAVSHEDPEVKFNWYVDGVEVH | |
| terminal lysine | NAKTKPREEQYNSTYRVVSVLTVDHHDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHSHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 941 |
| hIgG1- | NHKPSNTKVDKKVEPKSCDKTHTCPPCPAPEAAGGPSVFLFP | |
| LALA/DHS C- | PKPKDTLMISRTPEVTCVVVDVSHEDPEVKFNWYVDGVEVH | |
| terminal lysine | NAKTKPREEQYNSTYRVVSVLTVDHHDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHSHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 942 |
| hIgG1- | NHKPSNTKVDKKVEPKSCDKTHTCPPCPAPELAGAPSVFLFP | |
| LAGA/DHS C- | PKPKDTLMISRTPEVTCVVVDVSHEDPEVKFNWYVDGVEVH | |
| terminal lysine | NAKTKPREEQYNSTYRVVSVLTVDHHDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHSHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 943 |
| hIgG1- | NHKPSNTKVDKKVEPKSCDKTHTCPPCPAPEAAGAPSVFLFP | |
| LALAGA/DHS | PKPKDTLMISRTPEVTCVVVDVSHEDPEVKFNWYVDGVEVH | |
| C-terminal lysine | NAKTKPREEQYNSTYRVVSVLTVDHHDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHSHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 944 |
| hIgG1- | NHKPSNTKVDKKVEPKSCDKTHTCPPCPAPEAAGGPSVFLFP | |
| LALAPG/DHS | PKPKDTLMISRTPEVTCVVVDVSHEDPEVKFNWYVDGVEVH | |
| C-terminal lysine | NAKTKPREEQYNSTYRVVSVLTVDHHDWLNGKEYKCKVSN | |
| variant | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHSHYTQKSLSLSPGK | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 945 |
| IgG4-YTE C- | DHKPSNTKVDKRVESKYGPPCPSCPAPEFLGGPSVFLFPPKPK | |
| terminal lysine | DTLYITREPEVTCVVVDVSQEDPEVQFNWYVDGVEVHNAKT | |
| variant | KPREEQFNSTYRVVSVLTVLHQDWLNGKEYKCKVSNKGLPS | |
| | SIEKTISKAKGQPREPQVYTLPPSQEEMTKNQVSLTCLVKGFY | |
| | PSDIAVEWESNGQPENNYKTTPPVLDSDGSFFLYSRLTVDKS | |
| | RWQEGNVFSCSVMHEALHNHYTQKSLSLSLGK | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 946 |
| IgG4-SP/YTE C- | DHKPSNTKVDKRVESKYGPPCPPCPAPEFLGGPSVFLFPPKPK | |
| terminal lysine | DTLYITREPEVTCVVVDVSQEDPEVQFNWYVDGVEVHNAKT | |
| variant | KPREEQFNSTYRVVSVLTVLHQDWLNGKEYKCKVSNKGLPS | |
| | SIEKTISKAKGQPREPQVYTLPPSQEEMTKNQVSLTCLVKGFY | |
| | PSDIAVEWESNGQPENNYKTTPPVLDSDGSFFLYSRLTVDKS | |
| | RWQEGNVFSCSVMHEALHNHYTQKSLSLSLGK | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 947 |
| IgG4-SPLE/YTE | DHKPSNTKVDKRVESKYGPPCPPCPAPEELGGPSVFLFPPKPK | |
| C-terminal lysine | DTLYITREPEVTCVVVDVSQEDPEVQFNWYVDGVEVHNAKT | |
| variant | KPREEQFNSTYRVVSVLTVLHQDWLNGKEYKCKVSNKGLPS | |
| | SIEKTISKAKGQPREPQVYTLPPSQEEMTKNQVSLTCLVKGFY | |
| | PSDIAVEWESNGQPENNYKTTPPVLDSDGSFFLYSRLTVDKS | |
| | RWQEGNVFSCSVMHEALHNHYTQKSLSLSLGK | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 948 |
| IgG4-LS C- | DHKPSNTKVDKRVESKYGPPCPSCPAPEFLGGPSVFLFPPKPK | |
| terminal lysine | DTLMISRTPEVTCVVVDVSQEDPEVQFNWYVDGVEVHNAK | |
| variant | TKPREEQFNSTYRVVSVLTVLHQDWLNGKEYKCKVSNKGLP | |
| | SSIEKTISKAKGQPREPQVYTLPPSQEEMTKNQVSLTCLVKGF | |
| | YPSDIAVEWESNGQPENNYKTTPPVLDSDGSFFLYSRLTVDK | |
| | SRWQEGNVFSCSVLHEALHSHYTQKSLSLSLGK | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 949 |
| IgG4-SP/LS C- | DHKPSNTKVDKRVESKYGPPCPPCPAPEFLGGPSVFLFPPKPK | |
| terminal lysine | DTLMISRTPEVTCVVVDVSQEDPEVQFNWYVDGVEVHNAK | |
| variant | TKPREEQFNSTYRVVSVLTVLHQDWLNGKEYKCKVSNKGLP | |
| | SSIEKTISKAKGQPREPQVYTLPPSQEEMTKNQVSLTCLVKGF | |
| | YPSDIAVEWESNGQPENNYKTTPPVLDSDGSFFLYSRLTVDK | |
| | SRWQEGNVFSCSVLHEALHSHYTQKSLSLSLGK | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 950 |
| IgG4-SPLE/LS | DHKPSNTKVDKRVESKYGPPCPPCPAPEELGGPSVFLFPPKPK | |
| C-terminal lysine | DTLMISRTPEVTCVVVDVSQEDPEVQFNWYVDGVEVHNAK | |
| variant | TKPREEQFNSTYRVVSVLTVLHQDWLNGKEYKCKVSNKGLP | |
| | SSIEKTISKAKGQPREPQVYTLPPSQEEMTKNQVSLTCLVKGF | |
| | YPSDIAVEWESNGQPENNYKTTPPVLDSDGSFFLYSRLTVDK | |
| | SRWQEGNVFSCSVLHEALHSHYTQKSLSLSLGK | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 951 |
| IgG4-DHS C- | DHKPSNTKVDKRVESKYGPPCPSCPAPEFLGGPSVFLFPPKPK | |
| terminal lysine | DTLMISRTPEVTCVVVDVSQEDPEVQFNWYVDGVEVHNAK | |
| variant | TKPREEQFNSTYRVVSVLTVDHHDWLNGKEYKCKVSNKGL | |
| | PSSIEKTISKAKGQPREPQVYTLPPSQEEMTKNQVSLTCLVKG | |
| | FYPSDIAVEWESNGQPENNYKTTPPVLDSDGSFFLYSRLTVD | |
| | KSRWQEGNVFSCSVMHEALHSHYTQKSLSLSLGK | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 952 |
| IgG4-SP/DHS | DHKPSNTKVDKRVESKYGPPCPPCPAPEFLGGPSVFLFPPKPK | |
| C-terminal lysine | DTLMISRTPEVTCVVVDVSQEDPEVQFNWYVDGVEVHNAK | |
| variant | TKPREEQFNSTYRVVSVLTVDHHDWLNGKEYKCKVSNKGL | |
| | PSSIEKTISKAKGQPREPQVYTLPPSQEEMTKNQVSLTCLVKG | |
| | FYPSDIAVEWESNGQPENNYKTTPPVLDSDGSFFLYSRLTVD | |
| | KSRWQEGNVFSCSVMHEALHSHYTQKSLSLSLGK | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSSLGTKTYTCNV | NO: 953 |
| IgG4- | DHKPSNTKVDKRVESKYGPPCPPCPAPEELGGPSVFLFPPKPK | |
| SPLE/DHS C- | DTLMISRTPEVTCVVVDVSQEDPEVQFNWYVDGVEVHNAK | |
| terminal lysine | TKPREEQFNSTYRVVSVLTVDHHDWLNGKEYKCKVSNKGL | |
| variant | PSSIEKTISKAKGQPREPQVYTLPPSQEEMTKNQVSLTCLVKG | |
| | FYPSDIAVEWESNGQPENNYKTTPPVLDSDGSFFLYSRLTVD | |
| | KSRWQEGNVFSCSVMHEALHSHYTQKSLSLSLGK | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSNFGTQTYTCNV | NO: 954 |
| IgG2-YTE C- | DHKPSNTKVDKTVERKCCVECPPCPAPPVAGPSVFLFPPKPK | |
| terminal lysine | DTLYITREPEVTCVVVDVSHEDPEVQFNWYVDGVEVHNAKT | |
| variant | KPREEQFNSTFRVVSVLTVVHQDWLNGKEYKCKVSNKGLPA | |
| | PIEKTISKTKGQPREPQVYTLPPSREEMTKNQVSLTCLVKGFY | |
| | PSDIAVEWESNGQPENNYKTTPPMLDSDGSFFLYSKLTVDKS | |
| | RWQQGNVFSCSVMHEALHNHYTQKSLSLSPGK | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSNFGTQTYTCNV | NO: 955 |
| IgG2-LS C- | DHKPSNTKVDKTVERKCCVECPPCPAPPVAGPSVFLFPPKPK | |
| terminal lysine | DTLMISRTPEVTCVVVDVSHEDPEVQFNWYVDGVEVHNAK | |
| variant | TKPREEQFNSTFRVVSVLTVVHQDWLNGKEYKCKVSNKGLP | |
| | APIEKTISKTKGQPREPQVYTLPPSREEMTKNQVSLTCLVKGF | |
| | YPSDIAVEWESNGQPENNYKTTPPMLDSDGSFFLYSKLTVDK | |
| | SRWQQGNVFSCSVLHEALHSHYTQKSLSLSPGK | |
| Heavy Chain | ASTKGPSVFPLAPCSRSTSESTAALGCLVKDYFPEPVTVSWNS | SEQ ID |
| Constant Region | GALTSGVHTFPAVLQSSGLYSLSSVVTVPSSNFGTQTYTCNV | NO: 956 |
| IgG2-DHS C- | DHKPSNTKVDKTVERKCCVECPPCPAPPVAGPSVFLFPPKPK | |
| terminal lysine | DTLMISRTPEVTCVVVDVSHEDPEVQFNWYVDGVEVHNAK | |
| variant | TKPREEQFNSTFRVVSVLTVDHHDWLNGKEYKCKVSNKGLP | |
| | APIEKTISKTKGQPREPQVYTLPPSREEMTKNQVSLTCLVKGF | |
| | YPSDIAVEWESNGQPENNYKTTPPMLDSDGSFFLYSKLTVDK | |
| | SRWQQGNVFSCSVMHEALHSHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 957 |
| hIgG1-LA C- | NHKPSNTKVDKKVEPKSCDKTHTCPPCPAPELLGGPSVFLFP | |
| terminal lysine | PKPKDTLMISRTPEVTCVVVDVSHEDPEVKFNWYVDGVEVH | |
| variant | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHAHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 958 |
| hIgG1- | NHKPSNTKVDKKVEPKSCDKTHTCPPCPAPELLGGPSVFLFP | |
| N297A/LA C- | PKPKDTLMISRTPEVTCVVVDVSHEDPEVKFNWYVDGVEVH | |
| terminal lysine | NAKTKPREEQYASTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHAHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 959 |
| hIgG1- | NHKPSNTKVDKKVEPKSCDKTHTCPPCPAPELLGGPSVFLFP | |
| D265A/LA C- | PKPKDTLMISRTPEVTCVVVAVSHEDPEVKFNWYVDGVEVH | |
| terminal lysine | NAKTKPREEQYNSTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHAHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 960 |
| hIgG1- | NHKPSNTKVDKKVEPKSCDKTHTCPPCPAPEAAGGPSVFLFP | |
| LALA/LA C- | PKPKDTLMISRTPEVTCVVVDVSHEDPEVKFNWYVDGVEVH | |
| terminal lysine | NAKTKPREEQYNSTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHAHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 961 |
| hIgG1- | NHKPSNTKVDKKVEPKSCDKTHTCPPCPAPELAGAPSVFLFP | |
| LAGA/LA C- | PKPKDTLMISRTPEVTCVVVDVSHEDPEVKFNWYVDGVEVH | |
| terminal lysine | NAKTKPREEQYNSTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHAHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 962 |
| hIgG1- | NHKPSNTKVDKKVEPKSCDKTHTCPPCPAPEAAGAPSVFLFP | |
| LALAGA/LA C- | PKPKDTLMISRTPEVTCVVVDVSHEDPEVKFNWYVDGVEVH | |
| terminal lysine | NAKTKPREEQYNSTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHAHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 963 |
| hIgG1- | NHKPSNTKVDKKVEPKSCDKTHTCPPCPAPEAAGGPSVFLFP | |
| LALAPG/LA C- | PKPKDTLMISRTPEVTCVVVDVSHEDPEVKFNWYVDGVEVH | |
| terminal lysine | NAKTKPREEQYNSTYRVVSVLTVLHQDWLNGKEYKCKVSN | |
| variant | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVLHEALHAHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 964 |
| hIgG1-N434A | NHKPSNTKVDKKVEPKSCDKTHTCPPCPAPELLGGPSVFLFP | |
| C-terminal lysine | PKPKDTLMISRTPEVTCVVVDVSHEDPEVKFNWYVDGVEVH | |
| variant | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHAHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 965 |
| hIgG1-N297A/ | NHKPSNTKVDKKVEPKSCDKTHTCPPCPAPELLGGPSVFLFP | |
| N434A C- | PKPKDTLMISRTPEVTCVVVDVSHEDPEVKFNWYVDGVEVH | |
| terminal lysine | NAKTKPREEQYASTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHAHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 966 |
| hIgG1-D265A/ | NHKPSNTKVDKKVEPKSCDKTHTCPPCPAPELLGGPSVFLFP | |
| N434A C- | PKPKDTLMISRTPEVTCVVVAVSHEDPEVKFNWYVDGVEVH | |
| terminal lysine | NAKTKPREEQYNSTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHAHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 967 |
| hIgG1-LALA/ | NHKPSNTKVDKKVEPKSCDKTHTCPPCPAPEAAGGPSVFLFP | |
| N434A C- | PKPKDTLMISRTPEVTCVVVDVSHEDPEVKFNWYVDGVEVH | |
| terminal lysine | NAKTKPREEQYNSTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHAHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 968 |
| hIgG1-LAGA/ | NHKPSNTKVDKKVEPKSCDKTHTCPPCPAPELAGAPSVFLFP | |
| N434A C- | PKPKDTLMISRTPEVTCVVVDVSHEDPEVKFNWYVDGVEVH | |
| terminal lysine | NAKTKPREEQYNSTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHAHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 969 |
| hIgG1- | NHKPSNTKVDKKVEPKSCDKTHTCPPCPAPEAAGAPSVFLFP | |
| LALAGA/ | PKPKDTLMISRTPEVTCVVVDVSHEDPEVKFNWYVDGVEVH | |
| N434A C- | NAKTKPREEQYNSTYRVVSVLTVLHQDWLNGKEYKCKVSN | |
| terminal lysine | KALPAPIEKTISKAKGQPREPQVYTLPPSRDELTKNQVSLTCL | |
| variant | VKGFYPSDIAVEWESNGQPENNYKTTPPVLDSDGSFFLYSKL | |
| | TVDKSRWQQGNVFSCSVMHEALHAHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 970 |
| hIgG1- | NHKPSNTKVDKKVEPKSCDKTHTCPPCPAPEAAGGPSVFLFP | |
| LALAPG/ | PKPKDTLMISRTPEVTCVVVDVSHEDPEVKFNWYVDGVEVH | |
| N434A C- | NAKTKPREEQYNSTYRVVSVLTVLHQDWLNGKEYKCKVSN | |
| terminal lysine | KALGAPIEKTISKAKGQPREPQVYTLPPSRDELTKNQVSLTCL | |
| variant | VKGFYPSDIAVEWESNGQPENNYKTTPPVLDSDGSFFLYSKL | |
| | TVDKSRWQQGNVFSCSVMHEALHAHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 971 |
| hIgG1-N434W | NHKPSNTKVDKKVEPKSCDKTHTCPPCPAPELLGGPSVFLFP | |
| C-terminal lysine | PKPKDTLMISRTPEVTCVVVDVSHEDPEVKFNWYVDGVEVH | |
| variant | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHWHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | |
| hIgG1-N297A/ | NHKPSNTKVDKKVEPKSCDKTHTCPPCPAPELLGGPSVFLFP | |
| N434W C- | PKPKDTLMISRTPEVTCVVVDVSHEDPEVKFNWYVDGVEVH | NO: 972 |
| terminal lysine | NAKTKPREEQYASTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHWHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | |
| hIgG1-D265A/ | NHKPSNTKVDKKVEPKSCDKTHTCPPCPAPELLGGPSVFLFP | |
| N434W C- | PKPKDTLMISRTPEVTCVVVAVSHEDPEVKFNWYVDGVEVH | NO: 973 |
| terminal lysine | NAKTKPREEQYNSTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHWHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 974 |
| hIgG1-LALA/ | NHKPSNTKVDKKVEPKSCDKTHTCPPCPAPEAAGGPSVFLFP | |
| N434W C- | PKPKDTLMISRTPEVTCVVVDVSHEDPEVKFNWYVDGVEVH | |
| terminal lysine | NAKTKPREEQYNSTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHWHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 975 |
| hIgG1-LAGA/ | NHKPSNTKVDKKVEPKSCDKTHTCPPCPAPELAGAPSVFLFP | |
| N434W C- | PKPKDTLMISRTPEVTCVVVDVSHEDPEVKFNWYVDGVEVH | |
| terminal lysine | NAKTKPREEQYNSTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHWHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 976 |
| hIgG1- | NHKPSNTKVDKKVEPKSCDKTHTCPPCPAPEAAGAPSVFLFP | |
| LALAGA/ | PKPKDTLMISRTPEVTCVVVDVSHEDPEVKFNWYVDGVEVH | |
| N434W C- | NAKTKPREEQYNSTYRVVSVLTVLHQDWLNGKEYKCKVSN | |
| terminal lysine | KALPAPIEKTISKAKGQPREPQVYTLPPSRDELTKNQVSLTCL | |
| variant | VKGFYPSDIAVEWESNGQPENNYKTTPPVLDSDGSFFLYSKL | |
| | TVDKSRWQQGNVFSCSVMHEALHWHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 977 |
| hIgG1- | NHKPSNTKVDKKVEPKSCDKTHTCPPCPAPEAAGGPSVFLFP | |
| LALAPG/ | PKPKDTLMISRTPEVTCVVVDVSHEDPEVKFNWYVDGVEVH | |
| N434W C- | NAKTKPREEQYNSTYRVVSVLTVLHQDWLNGKEYKCKVSN | |
| terminal lysine | KALGAPIEKTISKAKGQPREPQVYTLPPSRDELTKNQVSLTCL | |
| variant | VKGFYPSDIAVEWESNGQPENNYKTTPPVLDSDGSFFLYSKL | |
| | TVDKSRWQQGNVFSCSVMHEALHWHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 978 |
| hIgG1-DQ C- | NHKPSNTKVDKKVEPKSCDKTHTCPPCPAPELLGGPSVFLFP | |
| terminal lysine | PKPKDTLMISRDPEVTCVVVDVSHEDPEVKFNWYVDGVEVH | |
| variant | NAKTKPREEQYNSTYRVVSVLQVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 979 |
| hIgG1- | NHKPSNTKVDKKVEPKSCDKTHTCPPCPAPELLGGPSVFLFP | |
| N297A/DQ C- | PKPKDTLMISRDPEVTCVVVDVSHEDPEVKFNWYVDGVEVH | |
| terminal lysine | NAKTKPREEQYASTYRVVSVLQVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 980 |
| hIgG1- | NHKPSNTKVDKKVEPKSCDKTHTCPPCPAPELLGGPSVFLFP | |
| D265A/DQ C- | PKPKDTLMISRDPEVTCVVVAVSHEDPEVKFNWYVDGVEVH | |
| terminal lysine | NAKTKPREEQYNSTYRVVSVLQVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 981 |
| hIgG1- | NHKPSNTKVDKKVEPKSCDKTHTCPPCPAPEAAGGPSVFLFP | |
| LALA/DQ C- | PKPKDTLMISRDPEVTCVVVDVSHEDPEVKFNWYVDGVEVH | |
| terminal lysine | NAKTKPREEQYNSTYRVVSVLQVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 982 |
| hIgG1- | NHKPSNTKVDKKVEPKSCDKTHTCPPCPAPELAGAPSVFLFP | |
| LAGA/DQ C- | PKPKDTLMISRDPEVTCVVVDVSHEDPEVKFNWYVDGVEVH | |
| terminal lysine | NAKTKPREEQYNSTYRVVSVLQVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 983 |
| hIgG1- | NHKPSNTKVDKKVEPKSCDKTHTCPPCPAPEAAGAPSVFLFP | |
| LALAGA/DQ | PKPKDTLMISRDPEVTCVVVDVSHEDPEVKFNWYVDGVEVH | |
| C-terminal lysine | NAKTKPREEQYNSTYRVVSVLQVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 984 |
| hIgG1- | NHKPSNTKVDKKVEPKSCDKTHTCPPCPAPEAAGGPSVFLFP | |
| LALAPG/DQ C- | PKPKDTLMISRDPEVTCVVVDVSHEDPEVKFNWYVDGVEVH | |
| terminal lysine | NAKTKPREEQYNSTYRVVSVLQVLHQDWLNGKEYKCKVSN | |
| variant | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 985 |
| hIgG1-DW C- | NHKPSNTKVDKKVEPKSCDKTHTCPPCPAPELLGGPSVFLFP | |
| terminal lysine | PKPKDTLMISRDPEVTCVVVDVSHEDPEVKFNWYVDGVEVH | |
| variant | NAKTKPREEQYNSTYRVVSVLWVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 986 |
| hIgG1- | NHKPSNTKVDKKVEPKSCDKTHTCPPCPAPELLGGPSVFLFP | |
| N297A/DW C- | PKPKDTLMISRDPEVTCVVVDVSHEDPEVKFNWYVDGVEVH | |
| terminal lysine | NAKTKPREEQYASTYRVVSVLWVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 987 |
| hIgG1- | NHKPSNTKVDKKVEPKSCDKTHTCPPCPAPELLGGPSVFLFP | |
| D265A/DW C- | PKPKDTLMISRDPEVTCVVVAVSHEDPEVKFNWYVDGVEVH | |
| terminal lysine | NAKTKPREEQYNSTYRVVSVLWVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 988 |
| hIgG1- | NHKPSNTKVDKKVEPKSCDKTHTCPPCPAPEAAGGPSVFLFP | |
| LALA/DW C- | PKPKDTLMISRDPEVTCVVVDVSHEDPEVKFNWYVDGVEVH | |
| terminal lysine | NAKTKPREEQYNSTYRVVSVLWVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 989 |
| hIgG1- | NHKPSNTKVDKKVEPKSCDKTHTCPPCPAPELAGAPSVFLFP | |
| LAGA/DW C- | PKPKDTLMISRDPEVTCVVVDVSHEDPEVKFNWYVDGVEVH | |
| terminal lysine | NAKTKPREEQYNSTYRVVSVLWVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 990 |
| hIgG1- | NHKPSNTKVDKKVEPKSCDKTHTCPPCPAPEAAGAPSVFLFP | |
| LALAGA/DW | PKPKDTLMISRDPEVTCVVVDVSHEDPEVKFNWYVDGVEVH | |
| C-terminal lysine | NAKTKPREEQYNSTYRVVSVLWVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 991 |
| hIgG1- | NHKPSNTKVDKKVEPKSCDKTHTCPPCPAPEAAGGPSVFLFP | |
| LALAPG/DW | PKPKDTLMISRDPEVTCVVVDVSHEDPEVKFNWYVDGVEVH | |
| C-terminal lysine | NAKTKPREEQYNSTYRVVSVLWVLHQDWLNGKEYKCKVSN | |
| variant | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 992 |
| hIgG1-YD C- | NHKPSNTKVDKKVEPKSCDKTHTCPPCPAPELLGGPSVFLFP | |
| terminal lysine | PKPKDTLYISRDPEVTCVVVDVSHEDPEVKFNWYVDGVEVH | |
| variant | NAKTKPREEQYNSTYRVVSVLTVLHQDWLNGKEYKCKVSN | |
| | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 993 |
| hIgG1- | NHKPSNTKVDKKVEPKSCDKTHTCPPCPAPELLGGPSVFLFP | |
| N297A/YD C- | PKPKDTLYISRDPEVTCVVVDVSHEDPEVKFNWYVDGVEVH | |
| terminal lysine | NAKTKPREEQYASTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 994 |
| hIgG1- | NHKPSNTKVDKKVEPKSCDKTHTCPPCPAPELLGGPSVFLFP | |
| D265A/YD C- | PKPKDTLYISRDPEVTCVVVAVSHEDPEVKFNWYVDGVEVH | |
| terminal lysine | NAKTKPREEQYNSTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 995 |
| hIgG1- | NHKPSNTKVDKKVEPKSCDKTHTCPPCPAPEAAGGPSVFLFP | |
| LALA/YD C- | PKPKDTLYISRDPEVTCVVVDVSHEDPEVKFNWYVDGVEVH | |
| terminal lysine | NAKTKPREEQYNSTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | |
| hIgG1- | NHKPSNTKVDKKVEPKSCDKTHTCPPCPAPELAGAPSVFLFP | |
| LAGA/YD C- | PKPKDTLYISRDPEVTCVVVDVSHEDPEVKFNWYVDGVEVH | NO: 996 |
| terminal lysine | NAKTKPREEQYNSTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 997 |
| hIgG1- | NHKPSNTKVDKKVEPKSCDKTHTCPPCPAPEAAGAPSVFLFP | |
| LALAGA/YD | PKPKDTLYISRDPEVTCVVVDVSHEDPEVKFNWYVDGVEVH | |
| C-terminal lysine | NAKTKPREEQYNSTYRVVSVLTVLHQDWLNGKEYKCKVSN | |
| variant | KALPAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | |
| hIgG1- | NHKPSNTKVDKKVEPKSCDKTHTCPPCPAPEAAGGPSVFLFP | |
| LALAPG/YD C- | PKPKDTLYISRDPEVTCVVVDVSHEDPEVKFNWYVDGVEVH | |
| terminal lysine | NAKTKPREEQYNSTYRVVSVLTVLHQDWLNGKEYKCKVSN | NO: 998 |
| variant | KALGAPIEKTISKAKGQPREPQVYTLPPSRDELTKNQVSLTCL | |
| | VKGFYPSDIAVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 999 |
| hIgG1-QVV C- | NHKPSNTKVDKKVEPKSCDKTHTCPPCPAPELLGGPSVFLFP | |
| terminal lysine | PKPKDTLMISRTPEVTCVVVDVSHEDPEVKFNWYVDGVEVH | |
| variant | NAKTKPREEQYNSTYRVVSVLQVLHVDWLNGKEYKCKVSN | |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 1000 |
| hIgG1- | NHKPSNTKVDKKVEPKSCDKTHTCPPCPAPELLGGPSVFLFP | |
| N297A/QVV C- | PKPKDTLMISRTPEVTCVVVDVSHEDPEVKFNWYVDGVEVH | |
| terminal lysine | NAKTKPREEQYASTYRVVSVLQVLHVDWLNGKEYKCKVSN | |
| variant | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 1001 |
| hIgG1- | NHKPSNTKVDKKVEPKSCDKTHTCPPCPAPELLGGPSVFLFP | |
| D265A/QVV C- | PKPKDTLMISRTPEVTCVVVAVSHEDPEVKFNWYVDGVEVH | |
| terminal lysine | NAKTKPREEQYNSTYRVVSVLQVLHVDWLNGKEYKCKVSN | |
| variant | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 1002 |
| hIgG1- | NHKPSNTKVDKKVEPKSCDKTHTCPPCPAPEAAGGPSVFLFP | |
| LALA/QVV C- | PKPKDTLMISRTPEVTCVVVDVSHEDPEVKFNWYVDGVEVH | |
| terminal lysine | NAKTKPREEQYNSTYRVVSVLQVLHVDWLNGKEYKCKVSN | |
| variant | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 1003 |
| hIgG1- | NHKPSNTKVDKKVEPKSCDKTHTCPPCPAPELAGAPSVFLFP | |
| LAGA/QVV C- | PKPKDTLMISRTPEVTCVVVDVSHEDPEVKFNWYVDGVEVH | |
| terminal lysine | NAKTKPREEQYNSTYRVVSVLQVLHVDWLNGKEYKCKVSN | |
| variant | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 1004 |
| hIgG1- | NHKPSNTKVDKKVEPKSCDKTHTCPPCPAPEAAGAPSVFLFP | |
| LALAGA/QVV | PKPKDTLMISRTPEVTCVVVDVSHEDPEVKFNWYVDGVEVH | |
| C-terminal lysine | NAKTKPREEQYNSTYRVVSVLQVLHVDWLNGKEYKCKVSN | |
| variant | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 1005 |
| hIgG1- | NHKPSNTKVDKKVEPKSCDKTHTCPPCPAPEAAGGPSVFLFP | |
| LALAPG/QVV | PKPKDTLMISRTPEVTCVVVDVSHEDPEVKFNWYVDGVEVH | |
| C-terminal lysine | NAKTKPREEQYNSTYRVVSVLQVLHVDWLNGKEYKCKVSN | |
| variant | KALGAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 1006 |
| hIgG1-DDRVV | NHKPSNTKVDKKVEPKSCDKTHTCPPCPAPELLGGPSVFLFP | |
| C-terminal lysine | PKPKDTLMISRDPEVTCVVVDVSHEDPEVKFNWYVDGVEVD | |
| variant | NAKTKPREEQYNSTYRVVSVLRVLHVDWLNGKEYKCKVSN | |
| | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | |
| hIgG1- | NHKPSNTKVDKKVEPKSCDKTHTCPPCPAPELLGGPSVFLFP | NO: 1007 |
| N297A/DDRVV | PKPKDTLMISRDPEVTCVVVDVSHEDPEVKFNWYVDGVEVD | |
| C-terminal lysine | NAKTKPREEQYASTYRVVSVLRVLHVDWLNGKEYKCKVSN | |
| variant | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 1008 |
| hIgG1- | NHKPSNTKVDKKVEPKSCDKTHTCPPCPAPELLGGPSVFLFP | |
| D265A/DDRVV | PKPKDTLMISRDPEVTCVVVAVSHEDPEVKFNWYVDGVEVD | |
| C-terminal lysine | NAKTKPREEQYNSTYRVVSVLRVLHVDWLNGKEYKCKVSN | |
| variant | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 1009 |
| hIgG1- | NHKPSNTKVDKKVEPKSCDKTHTCPPCPAPEAAGGPSVFLFP | |
| LALA/DDRVV | PKPKDTLMISRDPEVTCVVVDVSHEDPEVKFNWYVDGVEVD | |
| C-terminal lysine | NAKTKPREEQYNSTYRVVSVLRVLHVDWLNGKEYKCKVSN | |
| variant | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 1010 |
| hIgG1- | NHKPSNTKVDKKVEPKSCDKTHTCPPCPAPELAGAPSVFLFP | |
| LAGA/DDRVV | PKPKDTLMISRDPEVTCVVVDVSHEDPEVKFNWYVDGVEVD | |
| C-terminal lysine | NAKTKPREEQYNSTYRVVSVLRVLHVDWLNGKEYKCKVSN | |
| variant | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 1011 |
| hIgG1- | NHKPSNTKVDKKVEPKSCDKTHTCPPCPAPEAAGAPSVFLFP | |
| LALAGA/DDR | PKPKDTLMISRDPEVTCVVVDVSHEDPEVKFNWYVDGVEVD | |
| VV C-terminal | NAKTKPREEQYNSTYRVVSVLRVLHVDWLNGKEYKCKVSN | |
| lysine variant | KALPAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPGK | |
| Heavy Chain | ASTKGPSVFPLAPSSKSTSGGTAALGCLVKDYFPEPVTVSWN | SEQ ID |
| Constant Region | SGALTSGVHTFPAVLQSSGLYSLSSVVTVPSSSLGTQTYICNV | NO: 1012 |
| hIgG1- | NHKPSNTKVDKKVEPKSCDKTHTCPPCPAPEAAGGPSVFLFP | |
| LALAPG/DDR | PKPKDTLMISRDPEVTCVVVDVSHEDPEVKFNWYVDGVEVD | |
| VV C-terminal | NAKTKPREEQYNSTYRVVSVLRVLHVDWLNGKEYKCKVSN | |
| lysine variant | KALGAPIEKTISKAKGQPREPQVYTLPPSRDELTKNQVSLTCL | |
| | VKGFYPSDIVVEWESNGQPENNYKTTPPVLDSDGSFFLYSKL | |
| | TVDKSRWQQGNVFSCSVMHEALHNHYTQKSLSLSPGK | |
| Construct 114 | EVQVVESGGGLVQPGGSLRLSCAASGFTFSNYAMTWVRQSP | SEQ ID |
| Heavy Chain | GKGLEWVSLISGSGGLTKYADSVKGRFTISRDNSKKTLFLQM | NO: 1013 |
| | NSLRAEDTAAYYCVKDEGLTTGEYWGQGTQVTVSSASTKG | |
| | PSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKKVEPKSCDKTHTCPPCPAPEAAGGPSVFLFPPKPKD | |
| | TLYITREPEVTCVVVDVSHEDPEVKFNWYVDGVEVHNAKTK | |
| | PREEQYNSTYRVVSVLTVLHQDWLNGKEYKCKVSNKALPA | |
| | PIEKTISKAKGQPREPQVYTLPPSRDELTKNQVSLTCLVKGFY | |
| | PSDIAVEWESNGQPENNYKTTPPVLDSDGSFFLYSKLTVDKS | |
| | RWQQGNVFSCSVMHEALHNHYTQKSLSLSPGK | |
| Construct 114 | EVQVVESGGGLVQPGGSLRLSCAASGFTFSNYAMTWVRQSP | SEQ ID |
| Heavy Chain | GKGLEWVSLISGSGGLTKYADSVKGRFTISRDNSKKTLFLQM | NO: 1014 |
| without C- | NSLRAEDTAAYYCVKDEGLTTGEYWGQGTQVTVSSASTKG | |
| terminal lysine | PSVFPLAPSSKSTSGGTAALGCLVKDYFPEPVTVSWNSGALT | |
| | SGVHTFPAVLQSSGLYSLSSVVTVPSSSLGTQTYICNVNHKPS | |
| | NTKVDKKVEPKSCDKTHTCPPCPAPEAAGGPSVFLFPPKPKD | |
| | TLYITREPEVTCVVVDVSHEDPEVKFNWYVDGVEVHNAKTK | |
| | PREEQYNSTYRVVSVLTVLHQDWLNGKEYKCKVSNKALPA | |
| | PIEKTISKAKGQPREPQVYTLPPSRDELTKNQVSLTCLVKGFY | |
| | PSDIAVEWESNGQPENNYKTTPPVLDSDGSFFLYSKLTVDKS | |
| | RWQQGNVFSCSVMHEALHNHYTQKSLSLSPG | |
| Construct 114 | DIQMTQSPSSLSASVGDRVTITCRASQDIRNDLAWYQQKPGK | SEQ ID |
| light chain | APKRLIYASSSLQSGVPSRFSGSGSGTEFTLTISSLQPEDFATY | NO: 1015 |
| | YCLQHNNYPFTFGPGTKVDIKRTVAAPSVFIFPPSDEQLKSGT | |
| | ASVVCLLNNFYPREAKVQWKVDNALQSGNSQESVTEQDSK | |
| | DSTYSLSSTLTLSKADYEKHKVYACEVTHQGLSSPVTKSFNR | |
| | GEC | |
| IgG allotype | SRDEL | SEQ ID |
| variant | | NO: 1016 |
| IgG allotype | SREEM | SEQ ID |
| variant | | NO: 1017 |
| Amlitelimab | EVQLVESGGGLVQPGGSLRLSCAASGFTFSNYAMNWVRQAP | SEQ ID |
| Heavy Chain | GKGLEWVSTISGSGGATRYADSVKGRFTISRDNSRNTVYLQ | NO: 1018 |
| | MNSLRVEDTAVFYCTKDRLIMATVRGPYYYGMDVWGQGTT | |
| | VTVSSASTKGPSVFPLAPCSRSTSESTAALGCLVKDYFPEPVT | |
| | VSWNSGALTSGVHTFPAVLQSSGLYSLSSVVTVPSSSLGTKT | |
| | YTCNVDHKPSNTKVDKRVESKYGPPCPPCPAPEFEGGPSVFL | |
| | FPPKPKDTLMISRTPEVTCVVVDVSQEDPEVQFNWYVDGVE | |
| | VHNAKTKPREEQFNSTYRVVSVLTVLHQDWLNGKEYKCKV | |
| | SNKGLPSSIEKTISKAKGQPREPQVYTLPPSQEEMTKNQVSLT | |
| | CLVKGFYPSDIAVEWESNGQPENNYKTTPPVLDSDGSFFLYS | |
| | RLTVDKSRWQEGNVFSCSVMHEALHNHYTQKSLSLSLGK | |
| Amlitelimab | DIQMTQSPSSLSASVGDRVTITCRASQSISSYLNWYQQKPGK | SEQ ID |
| Light Chain | APNLLIYAASSLQSGVPSRFSGSGSETDFTLTISSLQPEDFATY | NO: 1019 |
| | YCQQSHSVSFTFGPGTKVDIKRTVAAPSVFIFPPSDEQLKSGT | |
| | ASVVCLLNNFYPREAKVQWKVDNALQSGNSQESVTEQDSK | |
| | DSTYSLSSTLTLSKADYEKHKVYACEVTHQGLSSPVTKSFNR | |
| | GEC | |

*SEQ ID NO: 913 is an hIgG1 sequence having a SRDEL (SEQ ID NO: 1016) allotype. The disclosure also encompasses SREEM (SEQ ID NO: 1017) allotype variants of any of the SRDEL (SEQ ID NO: 1016) allotype sequences set forth in the table above, and either a SRDEL (SEQ ID NO: 1016) allotype heavy chain constant region sequence or a SREEM (SEQ ID NO: 1017) allotype heavy chain constant region sequence may be included in an antibody described herein.

1. A composition comprising an isolated antibody, or antigen binding fragment thereof, that binds OX40L and a hyaluronidase, wherein the antibody, or antigen binding fragment thereof, comprises a CDR-H1 comprising the sequence set forth in SEQ ID NO: 1; a CDR-H2 comprising the sequence set forth in SEQ ID NO: 70; a CDR-H3 comprising the sequence set forth in SEQ ID NO: 175; a CDR-L1 comprising the sequence set forth in SEQ ID NO: 258; a CDR-L2 comprising the sequence set forth in SEQ ID NO: 335; and a CDR-L3 comprising the sequence set forth in SEQ ID NO: 368.

2. A composition comprising an isolated antibody, or antigen binding fragment thereof, that binds OX40L and a hyaluronidase, wherein the antibody, or antigen binding fragment thereof, comprises a CDR-H1 comprising the sequence set forth in SEQ ID NO: 44; a CDR-H2 comprising the sequence set forth in SEQ ID NO: 910; a CDR-H3 comprising the sequence set forth in SEQ ID NO: 911; a CDR-L1 comprising the sequence set forth in SEQ ID NO: 334; a CDR-L2 comprising the sequence set forth in SEQ ID NO: 361; and a CDR-L3 comprising the sequence set forth in SEQ ID NO: 912.

3. A composition comprising an isolated antibody, or antigen binding fragment thereof, that binds OX40L and a hyaluronidase, wherein the antibody, or antigen binding fragment thereof, comprises a VH sequence set forth in SEQ ID NO: 419 and a VL sequence set forth in SEQ ID NO: 489.

4. A composition comprising an isolated antibody, or antigen binding fragment thereof, that binds OX40L and a hyaluronidase, wherein the antibody, or antigen binding fragment thereof, comprises a VH sequence set forth in SEQ ID NO: 832 and a VL sequence set forth in SEQ ID NO: 833.

5. A combination comprising an isolated antibody, or antigen binding fragment thereof, that binds OX40L and a hyaluronidase, wherein the antibody, or antigen binding fragment thereof, comprises a CDR-H1 comprising the sequence set forth in SEQ ID NO:1; a CDR-H2 comprising the sequence set forth in SEQ ID NO: 70; a CDR-H3 comprising the sequence set forth in SEQ ID NO: 175; a CDR-L1 comprising the sequence set forth in SEQ ID NO: 258; a CDR-L2 comprising the sequence set forth in SEQ ID NO: 335; and a CDR-L3 comprising the sequence set forth in SEQ ID NO: 368.

6. A combination comprising an isolated antibody, or antigen binding fragment thereof, that binds OX40L and a hyaluronidase, wherein the antibody, or antigen binding fragment thereof, comprises a CDR-H1 comprising the sequence set forth in SEQ ID NO: 44; a CDR-H2 comprising the sequence set forth in SEQ ID NO: 910; a CDR-H3 comprising the sequence set forth in SEQ ID NO: 911; a CDR-L1 comprising the sequence set forth in SEQ ID NO: 334; a CDR-L2 comprising the sequence set forth in SEQ ID NO: 361; and a CDR-L3 comprising the sequence set forth in SEQ ID NO: 912.

7. A combination comprising an isolated antibody, or antigen binding fragment thereof, that binds OX40L and a hyaluronidase, wherein the antibody, or antigen binding fragment thereof, comprises a VH sequence set forth in SEQ ID NO: 419 and a VL sequence set forth in SEQ ID NO: 489.

8. A combination comprising an isolated antibody, or antigen binding fragment thereof, that binds OX40L and a hyaluronidase, wherein the antibody, or antigen binding fragment thereof, comprises a VH sequence set forth in SEQ ID NO: 832 and a VL sequence set forth in SEQ ID NO: 833.

9. A method of treating an inflammatory disorder or disease in a human patient comprising administering to the patient an isolated antibody, or antigen binding fragment thereof, that binds OX40L and a hyaluronidase, wherein the antibody, or antigen binding fragment thereof, comprises a CDR-H1 comprising the sequence set forth in SEQ ID NO:1; a CDR-H2 comprising the sequence set forth in SEQ ID NO: 70; a CDR-H3 comprising the sequence set forth in SEQ ID NO: 175; a CDR-L1 comprising the sequence set forth in SEQ ID NO: 258; a CDR-L2 comprising the sequence set forth in SEQ ID NO: 335; and a CDR-L3 comprising the sequence set forth in SEQ ID NO: 368.

10. A method of treating an inflammatory disorder or disease in a human patient comprising administering to the patient an isolated antibody, or antigen binding fragment thereof, that binds OX40L and a hyaluronidase, wherein the antibody, or antigen binding fragment thereof, comprises a CDR-H1 comprising the sequence set forth in SEQ ID NO: 44; a CDR-H2 comprising the sequence set forth in SEQ ID NO: 910; a CDR-H3 comprising the sequence set forth in SEQ ID NO: 911; a CDR-L1 comprising the sequence set forth in SEQ ID NO: 334; a CDR-L2 comprising the sequence set forth in SEQ ID NO: 361; and a CDR-L3 comprising the sequence set forth in SEQ ID NO: 912.

11. A method of treating an inflammatory disorder or disease in a human patient comprising administering to the patient an isolated antibody, or antigen binding fragment thereof, that binds OX40L and a hyaluronidase, wherein the antibody, or antigen binding fragment thereof, comprises a VH sequence set forth in SEQ ID NO: 419 and a VL sequence set forth in SEQ ID NO: 489.

12. A method of treating an inflammatory disorder or disease in a human patient comprising administering to the patient an isolated antibody, or antigen binding fragment thereof, that binds OX40L and a hyaluronidase, wherein the antibody, or antigen binding fragment thereof, comprises a VH sequence set forth in SEQ ID NO: 832 and a VL sequence set forth in SEQ ID NO: 833.

13. The composition of any one of claims 1-4, the combination of any one of claims 5-8, or the method of any one of claims 9-12, wherein the antibody, or antigen binding fragment thereof, is a humanized, human, or chimeric antibody.

14. The composition of any one of claims 1-4, the combination of any one of claims 5-8, or the method of any one of claims 9-12, wherein the antibody, or antigen binding fragment thereof, is a humanized antibody.

15. The composition of any one of claims 1-4, the combination of any one of claims 5-8, or the method of any one of claims 9-12, wherein the antibody, or antigen binding fragment thereof, comprises a heavy chain human constant region of a class selected from IgG, IgA, IgD, IgE, and IgM.

16. The composition of any one of claims 1-4, the combination of any one of claims 5-8, or the method of any one of claims 9-12, wherein the antibody, or antigen binding fragment thereof, comprises a human Fc region comprising a human heavy chain constant region of the class IgG and a subclass selected from IgG1, IgG2, IgG3, and IgG4.

17. The composition of any one of claims 1-4, the combination of any one of claims 5-8, or the method of any one of claims 9-12, wherein the antibody, or antigen binding fragment thereof, comprises a human IgG1 Fc region.

18. The composition of any one of claims 1-4, the combination of any one of claims 5-8, or the method of any one of claims 9-12, wherein the antibody, or antigen binding fragment thereof, comprises a human IgG1 Fc region with LALA and YTE mutations.

19. The composition of any one of claims 1-4, the combination of any one of claims 5-8, or the method of any one of claims 9-12, wherein the antibody, or antigen binding fragment thereof, comprises a human IgG1 Fc region with LALA mutations at positions 235 and 236 (L235A/L236A) and YTE mutations at positions 253, 255, and 257 (M253Y/S255T/T257E) by direct numbering.

20. The composition of any one of claims 1-4, the combination of any one of claims 5-8, or the method of any one of claims 9-12, wherein the antibody, or antigen binding fragment thereof, comprises a human Fc region comprising a human IgG sequence selected from a sequence set forth in any one of SEQ ID NOs: 637-737 and 913-1012.

21. The composition of any one of claims 1-4, the combination of any one of claims 5-8, or the method of any one of claims 9-12, wherein the antibody, or antigen binding fragment thereof, comprises a heavy chain comprising a constant heavy chain region having a means for extending the half-life of the antibody.

22. The composition of any one of claims 1-4, the combination of any one of claims 5-8, or the method of any one of claims 9-12, wherein the antibody, or antigen binding fragment thereof, comprises a heavy chain comprising a constant heavy chain sequence set forth in SEQ ID NO: 651 or SEQ ID NO: 927.

23. The composition of any one of claims 1-4, the combination of any one of claims 5-8, or the method of any one of claims 9-12, wherein the antibody, or antigen binding fragment thereof, comprises a light chain comprising a constant light chain sequence set forth in SEQ ID NO: 738.

24. The composition of claim 1 or 3, the combination of claim 5 or 7, or the method of claim 9 or 11, wherein the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 927 or SEQ ID NO: 651, and a constant light chain sequence set forth in SEQ ID NO: 738.

25. The composition of any one of claims 1-4, the combination of any one of claims 5-8, or the method of any one of claims 9-12, wherein the Fc region of the antibody binds to Neonatal Fc receptor (FcRn).

26. The composition of any one of claims 1-4, the combination of any one of claims 5-8, or the method of any one of claims 9-12, wherein the Fc region of the antibody binds an FcRn with higher affinity at pH 6.0 compared to an antibody comprising a wild-type Fc region.

27. The composition of any one of claims 1-4, the combination of any one of claims 5-8, or the method of any one of claims 9-12, wherein the Fc region of the antibody binds to FcRn with a K_{D} of <1×10⁻⁷ M at pH 6.0.

28. The composition of any one of claims 1-4, the combination of any one of claims 5-8, or the method of any one of claims 9-12, wherein the antibody, or antigen binding fragment thereof, is a monoclonal antibody.

29. The composition of any one of claims 1-4, the combination of any one of claims 5-8, or the method of any one of claims 9-12, wherein the antibody, or antigen binding fragment thereof, binds an OX40L sequence set forth in SEQ ID NO: 739.

30. The composition of any one of claims 1-4, the combination of any one of claims 5-8, or the method of any one of claims 9-12, wherein the hyaluronidase is a recombinant human hyaluronidase.

31. The composition, combination, or method of claim 30, wherein the recombinant human hyaluronidase comprises the amino acid sequence set forth in any one of SEQ ID NOs: 843-852 and 854-909.

32. The composition, combination, or method of claim 30, wherein the recombinant human hyaluronidase is rHuPH20.

33. The composition, combination, or method of claim 32, wherein the rHuPH20 formulation is ENHANZE®.

34. The combination of any one of claims 5-8 or the method of any one of claims 9-12, wherein the hyaluronidase and antibody, or antigen binding fragment thereof, are administered in separate formulations.

35. The combination or method of claim 34, wherein the hyaluronidase and antibody, or antigen binding fragment thereof, are administered simultaneously in separate formulations.

36. The combination or method of claim 34, wherein the hyaluronidase is administered to the patient prior to administration of the antibody, or antigen binding fragment thereof.

37. The combination or method of claim 34, wherein the hyaluronidase is administered to the patient after administration of the antibody, or antigen binding fragment thereof.

38. The composition of any one of claims 1-4, the combination of any one of claims 5-8, or the method of any one of claims 9-12, wherein the hyaluronidase and antibody, or antigen binding fragment thereof, are mixed and administered in a single formulation.

39. The composition of any one of claims 1-4, the combination of any one of claims 5-8, or the method of any one of claims 9-12, wherein the hyaluronidase and antibody, or antigen binding fragment thereof, are administered by subcutaneous injection.

40. The composition of any one of claims 1-4, the combination of any one of claims 5-8, or the method of any one of claims 9-12, wherein the hyaluronidase and antibody, or antigen binding fragment thereof, are administered by intravenous injection.

41. The composition of any one of claims 1-4, the combination of any one of claims 5-8, or the method of any one of claims 9-12, wherein the hyaluronidase is rHuPH20 administered at a concentration of 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, or 40,000 units.

42. The method of any one of claims 9-12, wherein the inflammatory disorder or disease is atopic dermatitis.

43. The method of claim 42, wherein the treatment reduces disease severity in the patient and wherein disease severity is assessed by an atopic dermatitis disease severity outcome measure.

44. The method of any one of claims 9-12, wherein the inflammatory disorder or disease is selected from the group consisting of asthma, chronic sinusitis with nasal polyps, Chronic Rhinosinusitis without Nasal Polyps (CRSsNP), eosinophilic esophagitis (EoE), Churg-Strauss syndrome/Eosinophilic granulomatosis with polyangiitis (EGPA), Prurigo Nodularis (PN), Chronic Spontaneous Urticaria (CSU), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), lupus, rheumatoid arthritis (RA), psoriasis, ulcerative colitis, Crohn's disease, hidradenitis suppurativa, celiac disease, idiopathic pulmonary fibrosis, alopecia areata, allergic rhinitis, eosinophilic fasciitis, scleromyxedema, scleredema, nephrogenic systemic fibrosis, and systemic sclerosis.

45. The method of any one of claims 9-12, wherein the inflammatory disorder or disease is an Eosinophilic gastrointestinal disorder or disease (EGID) selected from the group consisting of Eosinophilic Gastritis (EoG), Eosinophilic Enteritis (EoN), Eosinophilic Colitis (EoC), and Eosinophilic Gastroenteritis (EGE).

46. A kit for treating an inflammatory disorder or disease in a human patient, the kit comprising:
(a) a dose of an isolated antibody, or antigen binding fragment thereof, that binds OX40L, wherein the antibody comprises a VH sequence set forth in SEQ ID NO: 419 and a VL sequence set forth in SEQ ID NO: 489;
(b) a dose of a hyaluronidase; and
(c) instructions.

47. The kit of claim 46, wherein the hyaluronidase is a recombinant human hyaluronidase.

48. The kit of claim 47, wherein the recombinant human hyaluronidase comprises the amino acid sequence set forth in any one of SEQ ID NOs: 843-852 and 854-909.

49. The kit of claim 47, wherein the recombinant human hyaluronidase is rHuPH20.

50. The kit of claim 49, wherein the rHuPH20 formulation is ENHANZE®.

51. The kit of claim 46 for use in treating an inflammatory disorder or disease.

52. The kit of claim 51, wherein the inflammatory disorder or disease is atopic dermatitis.

53. The kit of claim 51, wherein the inflammatory disorder or disease is selected from the group consisting of asthma, chronic sinusitis with nasal polyps, Chronic Rhinosinusitis without Nasal Polyps (CRSsNP), eosinophilic esophagitis (EoE), Churg-Strauss syndrome/Eosinophilic granulomatosis with polyangiitis (EGPA), Prurigo Nodularis (PN), Chronic Spontaneous Urticaria (CSU), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), Chronic Pruritis of Unknown Origin (CPUO), Bullous Pemphigoid (BP), Cold Inducible Urticaria (ColdU), Allergic Fungal Rhinosinusitis (AFRS), Allergic Bronchopulmonary Aspergillosis (ABPA), Chronic Obstructive Pulmonary Disease (COPD), lupus, rheumatoid arthritis (RA), psoriasis, ulcerative colitis, Crohn's disease, hidradenitis suppurativa, celiac disease, idiopathic pulmonary fibrosis, alopecia areata, allergic rhinitis, eosinophilic fasciitis, scleromyxedema, scleredema, nephrogenic systemic fibrosis, and systemic sclerosis.

54. The kit of claim 51, wherein the inflammatory disorder or disease is an Eosinophilic gastrointestinal disorder or disease (EGID) selected from the group consisting of Eosinophilic Gastritis (EoG), Eosinophilic Enteritis (EoN), Eosinophilic Colitis (EoC), and Eosinophilic Gastroenteritis (EGE).

55. The kit of any one of claims 46-54, wherein the antibody, or antigen binding fragment thereof, comprises a VH sequence set forth in SEQ ID NO: 419, a VL sequence set forth in SEQ ID NO: 489, a constant heavy chain sequence set forth in SEQ ID NO: 927, and a constant light chain sequence set forth in SEQ ID NO: 738.


SEQUENCE LISTING (1019 sequences)

SEQ ID NO: 1 - protein, 5 aa, synthetic construct
NYAMT

SEQ ID NO: 2 - protein, 5 aa, synthetic construct
SYAMN

SEQ ID NO: 3 - protein, 5 aa, synthetic construct
SNYMS

SEQ ID NO: 4 - protein, 5 aa, synthetic construct
NYWMH

SEQ ID NO: 5 - protein, 5 aa, synthetic construct
NAWMS

SEQ ID NO: 6 - protein, 5 aa, synthetic construct
NAWMT

SEQ ID NO: 7 - protein, 5 aa, synthetic construct
YAWMS

SEQ ID NO: 8 - protein, 5 aa, synthetic construct
NYDIN

SEQ ID NO: 9 - protein, 5 aa, synthetic construct
SYAMY

SEQ ID NO: 10 - protein, 5 aa, synthetic construct
SYAMT

SEQ ID NO: 11 - protein, 5 aa, synthetic construct
SYAMS

SEQ ID NO: 12 - protein, 5 aa, synthetic construct
TYAMT

SEQ ID NO: 13 - protein, 5 aa, synthetic construct
SHAMT

SEQ ID NO: 14 - protein, 5 aa, synthetic construct
IYAMT

SEQ ID NO: 15 - protein, 5 aa, synthetic construct
SYWMH

SEQ ID NO: 16 - protein, 5 aa, synthetic construct
SFWMH

SEQ ID NO: 17 - protein, 5 aa, synthetic construct
SAWMS

SEQ ID NO: 18 - protein, 5 aa, synthetic construct
NAWMR

SEQ ID NO: 19 - protein, 5 aa, synthetic construct
YAWMT

SEQ ID NO: 20 - protein, 5 aa, synthetic construct
NAWMN

SEQ ID NO: 21 - protein, 5 aa, synthetic construct
TAWMS

SEQ ID NO: 22 - protein, 7 aa, synthetic construct
GFTFSNY

SEQ ID NO: 23 - protein, 7 aa, synthetic construct
GFRFSSY

SEQ ID NO: 24 - protein, 7 aa, synthetic construct
GFPVSSN

SEQ ID NO: 25 - protein, 7 aa, synthetic construct
GFTFSYA

SEQ ID NO: 26 - protein, 7 aa, synthetic construct
GYTFTNY

SEQ ID NO: 27 - protein, 7 aa, synthetic construct
GFTFSSY

SEQ ID NO: 28 - protein, 7 aa, synthetic construct
GFTFRNY

SEQ ID NO: 29 - protein, 7 aa, synthetic construct
GFTFNTY

SEQ ID NO: 30 - protein, 7 aa, synthetic construct
GFTFSSH

SEQ ID NO: 31 - protein, 7 aa, synthetic construct
GFTFGNY

SEQ ID NO: 32 - protein, 7 aa, synthetic construct
GFTFTNY

SEQ ID NO: 33 - protein, 7 aa, synthetic construct
GFTFSIY

SEQ ID NO: 34 - protein, 7 aa, synthetic construct
GFTFRSY

SEQ ID NO: 35 - protein, 7 aa, synthetic construct
GLTFSNY

SEQ ID NO: 36 - protein, 7 aa, synthetic construct
GFTFSSF

SEQ ID NO: 37 - protein, 7 aa, synthetic construct
GFTFHNA

SEQ ID NO: 38 - protein, 7 aa, synthetic construct
GFTFNSA

SEQ ID NO: 39 - protein, 7 aa, synthetic construct
GFIFNNA

SEQ ID NO: 40 - protein, 7 aa, synthetic construct
GFTFNYA

SEQ ID NO: 41 - protein, 7 aa, synthetic construct
GFTFINA

SEQ ID NO: 42 - protein, 7 aa, synthetic construct
GFTFSTA

SEQ ID NO: 43 - protein, 7 aa, synthetic construct
GFIFSNA

SEQ ID NO: 44 - protein, 8 aa, synthetic construct
GFTFSNYA

SEQ ID NO: 45 - protein, 8 aa, synthetic construct
GFRFSSYA

SEQ ID NO: 46 - protein, 8 aa, synthetic construct
GFPVSSNY

SEQ ID NO: 47 - protein, 8 aa, synthetic construct
GFTFSNYW

SEQ ID NO: 48 - protein, 8 aa, synthetic construct
GFTFSNAW

SEQ ID NO: 49 - protein, 8 aa, synthetic construct
GFTFNNAW

SEQ ID NO: 50 - protein, 8 aa, synthetic construct
GFTFSYAW

SEQ ID NO: 51 - protein, 8 aa, synthetic construct
GYTFTNYD

SEQ ID NO: 52 - protein, 8 aa, synthetic construct
GFTFSSYA

SEQ ID NO: 53 - protein, 8 aa, synthetic construct
GFTFRNYA

SEQ ID NO: 54 - protein, 8 aa, synthetic construct
GFTFNTYA

SEQ ID NO: 55 - protein, 8 aa, synthetic construct
GFTFSSHA

SEQ ID NO: 56 - protein, 8 aa, synthetic construct
GFTFGNYA

SEQ ID NO: 57 - protein, 8 aa, synthetic construct
GFTFTNYA

SEQ ID NO: 58 - protein, 8 aa, synthetic construct
GFTFSIYA

SEQ ID NO: 59 - protein, 8 aa, synthetic construct
GFTFRSYW

SEQ ID NO: 60 - protein, 8 aa, synthetic construct
GLTFSNYW

SEQ ID NO: 61 - protein, 8 aa, synthetic construct
GFTFSSFW

SEQ ID NO: 62 - protein, 8 aa, synthetic construct
GFTFSSYW

SEQ ID NO: 63 - protein, 8 aa, synthetic construct
GFTFHNAW

SEQ ID NO: 64 - protein, 8 aa, synthetic construct
GFTFNSAW

SEQ ID NO: 65 - protein, 8 aa, synthetic construct
GFIFNNAW

SEQ ID NO: 66 - protein, 8 aa, synthetic construct
GFTFNYAW

SEQ ID NO: 67 - protein, 8 aa, synthetic construct
GFTFINAW

SEQ ID NO: 68 - protein, 8 aa, synthetic construct
GFTFSTAW

SEQ ID NO: 69 - protein, 8 aa, synthetic construct
GFIFSNAW

SEQ ID NO: 70 - protein, 17 aa, synthetic construct
LISGSGGLTKYADSVKG

SEQ ID NO: 71 - protein, 16 aa, synthetic construct
IIYSRDSTHYADSVKG

SEQ ID NO: 72 - protein, 17 aa, synthetic construct
RINSDGISTRYADSVKG

SEQ ID NO: 73 - protein, 19 aa, synthetic construct
RIKSNSYGGTTDYAAPAIG

SEQ ID NO: 74 - protein, 19 aa, synthetic construct
RIKSISYGGTTDSAAPVKG

SEQ ID NO: 75 - protein, 19 aa, synthetic construct
RIKSETDGGTTDYAAPVKG

SEQ ID NO: 76 - protein, 17 aa, synthetic construct
WMNPHSGQTGYAQKFQG

SEQ ID NO: 77 - protein, 17 aa, synthetic construct
LITGSGGSTHYADSVKG

SEQ ID NO: 78 - protein, 17 aa, synthetic construct
LISGSGGITKYADSVKG

SEQ ID NO: 79 - protein, 17 aa, synthetic construct
LISGSGGITHYADSVKG

SEQ ID NO: 80 - protein, 17 aa, synthetic construct
LISGSGGVTHYADSVKG

SEQ ID NO: 81 - protein, 17 aa, synthetic construct
LISGSGAITHYADSVKG

SEQ ID NO: 82 - protein, 17 aa, synthetic construct
LISGSGGLTNYADSVKG

SEQ ID NO: 83 - protein, 17 aa, synthetic construct
LISGSGGSTHYADSVKG

SEQ ID NO: 84 - protein, 17 aa, synthetic construct
LISGSGGLTKYADSVRG

SEQ ID NO: 85 - protein, 17 aa, synthetic construct
LISGSGSLTKYADSVKG

SEQ ID NO: 86 - protein, 17 aa, synthetic construct
LISGGGGLTYYADSVKG

SEQ ID NO: 87 - protein, 17 aa, synthetic construct
LISGSGGLTYYADSVKG

SEQ ID NO: 88 - protein, 17 aa, synthetic construct
RIDNNGINTRYADSVKG

SEQ ID NO: 89 - protein, 17 aa, synthetic construct
RISSDGISTRYADSVKG

SEQ ID NO: 90 - protein, 17 aa, synthetic construct
RINNDGISTRYADSVKG

SEQ ID NO: 91 - protein, 17 aa, synthetic construct
RINSDGFSTRYADSVKG

SEQ ID NO: 92 - protein, 19 aa, synthetic construct
RIKGVTAGGTADYAAPVKG

SEQ ID NO: 93 - protein, 19 aa, synthetic construct
RVKSVTAGGTTDYAAPVKG

SEQ ID NO: 94 - protein, 19 aa, synthetic construct
RIKSKTDGGTTDYAAPVKG

SEQ ID NO: 95 - protein, 19 aa, synthetic construct
RIKSVTTGGTTDYAAPVKG

SEQ ID NO: 96 - protein, 19 aa, synthetic construct
RIKSVTAGGTTDYAAPVKG

SEQ ID NO: 97 - protein, 19 aa, synthetic construct
RIKSVTAGGTTDFPAPVKG

SEQ ID NO: 98 - protein, 19 aa, synthetic construct
HIKSETDGGTTDYAAPVKG

SEQ ID NO: 99 - protein, 19 aa, synthetic construct
RIKRKTDGGTTDYAAPVQG

SEQ ID NO: 100 - protein, 19 aa, synthetic construct
RIKSLTDGGTTDYAAPVKG

SEQ ID NO: 101 - protein, 19 aa, synthetic construct
RIKRKTDGGTREYAAPVKG

SEQ ID NO: 102 - protein, 19 aa, synthetic construct
RIKRNTDGGTTDYAAPVKG

SEQ ID NO: 103 - protein, 19 aa, synthetic construct
RIKTNTDGGTTDYAAPVKG

SEQ ID NO: 104 - protein, 19 aa, synthetic construct
RIKSKTYGGTTDYDAPVKG

SEQ ID NO: 105 - protein, 19 aa, synthetic construct
RIKSQTYGGTTDYAAPVKG

SEQ ID NO: 106 - protein, 19 aa, synthetic construct
RIKSKTYGGTTDYAAPVKG

SEQ ID NO: 107 - protein, 19 aa, synthetic construct
RIKRKTDGGTRDYAAPVKG

SEQ ID NO: 108 - protein, 19 aa, synthetic construct
RIKRETDGGTTDYASPVKG

SEQ ID NO: 109 - protein, 19 aa, synthetic construct
RIKRKSDGGTTDYAAPVKG

SEQ ID NO: 110 - protein, 19 aa, synthetic construct
RIKSEIDGGTTDYAAPVKG

SEQ ID NO: 111 - protein, 6 aa, synthetic construct
SGSGGL

SEQ ID NO: 112 - protein, 5 aa, synthetic construct
YSRDS

SEQ ID NO: 113 - protein, 6 aa, synthetic construct
NSDGIS

SEQ ID NO: 114 - protein, 8 aa, synthetic construct
KSNSYGGT

SEQ ID NO: 115 - protein, 8 aa, synthetic construct
KSISYGGT

SEQ ID NO: 116 - protein, 8 aa, synthetic construct
KSETDGGT

SEQ ID NO: 117 - protein, 6 aa, synthetic construct
NPHSGQ

SEQ ID NO: 118 - protein, 6 aa, synthetic construct
TGSGGS

SEQ ID NO: 119 - protein, 6 aa, synthetic construct
SGSGGI

SEQ ID NO: 120 - protein, 6 aa, synthetic construct
SGSGGV

SEQ ID NO: 121 - protein, 6 aa, synthetic construct
SGSGAI

SEQ ID NO: 122 - protein, 6 aa, synthetic construct
SGSGGS

SEQ ID NO: 123 - protein, 6 aa, synthetic construct
SGSGSL

SEQ ID NO: 124 - protein, 6 aa, synthetic construct
SGGGGL

SEQ ID NO: 125 - protein, 6 aa, synthetic construct
DNNGIN

SEQ ID NO: 126 - protein, 6 aa, synthetic construct
SSDGIS

SEQ ID NO: 127 - protein, 6 aa, synthetic construct
NNDGIS

SEQ ID NO: 128 - protein, 6 aa, synthetic construct
NSDGFS

SEQ ID NO: 129 - protein, 8 aa, synthetic construct
KGVTAGGT

SEQ ID NO: 130 - protein, 8 aa, synthetic construct
KSVTAGGT

SEQ ID NO: 131 - protein, 8 aa, synthetic construct
KSKTDGGT

SEQ ID NO: 132 - protein, 8 aa, synthetic construct
KSVTTGGT

SEQ ID NO: 133 - protein, 8 aa, synthetic construct
KRKTDGGT

SEQ ID NO: 134 - protein, 8 aa, synthetic construct
KSLTDGGT

SEQ ID NO: 135 - protein, 8 aa, synthetic construct
KRNTDGGT

SEQ ID NO: 136 - protein, 8 aa, synthetic construct
KRNTDGGT

SEQ ID NO: 137 - protein, 8 aa, synthetic construct
KSKTYGGT

SEQ ID NO: 138 - protein, 8 aa, synthetic construct
KSQTYGGT

SEQ ID NO: 139 - protein, 8 aa, synthetic construct
KRETDGGT

SEQ ID NO: 140 - protein, 8 aa, synthetic construct
KRKSDGGT

SEQ ID NO: 141 - protein, 8 aa, synthetic construct
KSEIDGGT

SEQ ID NO: 142 - protein, 8 aa, synthetic construct
ISGSGGLT

SEQ ID NO: 143 - protein, 7 aa, synthetic construct
IYSRDST

SEQ ID NO: 144 - protein, 8 aa, synthetic construct
INSDGIST

SEQ ID NO: 145 - protein, 10 aa, synthetic construct
IKSNSYGGTT

SEQ ID NO: 146 - protein, 10 aa, synthetic construct
IKSISYGGTT

SEQ ID NO: 147 - protein, 10 aa, synthetic construct
IKSETDGGTT

SEQ ID NO: 148 - protein, 8 aa, synthetic construct
MNPHSGQT

SEQ ID NO: 149 - protein, 8 aa, synthetic construct
ITGSGGST

SEQ ID NO: 150 - protein, 8 aa, synthetic construct
ISGSGGIT

SEQ ID NO: 151 - protein, 8 aa, synthetic construct
ISGSGGVT

SEQ ID NO: 152 - protein, 8 aa, synthetic construct
ISGSGAIT

SEQ ID NO: 153 - protein, 8 aa, synthetic construct
ISGSGGST

SEQ ID NO: 154 - protein, 8 aa, synthetic construct
ISGSGSLT

SEQ ID NO: 155 - protein, 8 aa, synthetic construct
ISGGGGLT

SEQ ID NO: 156 - protein, 8 aa, synthetic construct
IDNNGINT

SEQ ID NO: 157 - protein, 8 aa, synthetic construct
ISSDGIST

SEQ ID NO: 158 - protein, 8 aa, synthetic construct
INNDGIST

SEQ ID NO: 159 - protein, 8 aa, synthetic construct
INSDGFST

SEQ ID NO: 160 - protein, 10 aa, synthetic construct
IKGVTAGGTA

SEQ ID NO: 161 - protein, 10 aa, synthetic construct
VKSVTAGGTT

SEQ ID NO: 162 - protein, 10 aa, synthetic construct
IKSKTDGGTT

SEQ ID NO: 163 - protein, 10 aa, synthetic construct
IKSVTTGGTT

SEQ ID NO: 164 - protein, 10 aa, synthetic construct
IKSVTAGGTT

SEQ ID NO: 165 - protein, 10 aa, synthetic construct
IKRKTDGGTT

SEQ ID NO: 166 - protein, 10 aa, synthetic construct
IKSLTDGGTT

SEQ ID NO: 167 - protein, 10 aa, synthetic construct
IKRKTDGGTR

SEQ ID NO: 168 - protein, 10 aa, synthetic construct
IKRNTDGGTT

SEQ ID NO: 169 - protein, 10 aa, synthetic construct
IKTNTDGGTT

SEQ ID NO: 170 - protein, 10 aa, synthetic construct
IKSKTYGGTT

SEQ ID NO: 171 - protein, 10 aa, synthetic construct
IKSQTYGGTT

SEQ ID NO: 172 - protein, 10 aa, synthetic construct
IKRETDGGTT

SEQ ID NO: 173 - protein, 10 aa, synthetic construct
IKRKSDGGTT

SEQ ID NO: 174 - protein, 10 aa, synthetic construct
IKSEIDGGTT

SEQ ID NO: 175 - protein, 9 aa, synthetic construct
DEGLTTGEY

SEQ ID NO: 176 - protein, 9 aa, synthetic construct
ERWEPSFHY

SEQ ID NO: 177 - protein, 12 aa, synthetic construct
TLTYYYGAGSDY

SEQ ID NO: 178 - protein, 9 aa, synthetic construct
DFQLELPDY

SEQ ID NO: 179 - protein, 9 aa, synthetic construct
DHPKFGFDF

SEQ ID NO: 180 - protein, 12 aa, synthetic construct
GSVWKYGGSLDF

SEQ ID NO: 181 - protein, 11 aa, synthetic construct
GSSWNVYFFDF

SEQ ID NO: 182 - protein, 16 aa, synthetic construct
EGYDLWSGDSSNWLDP

SEQ ID NO: 183 - protein, 16 aa, synthetic construct
EGYDLWSGESSNWLDP

SEQ ID NO: 184 - protein, 16 aa, synthetic construct
EGYDLWSGHSSNWLDP

SEQ ID NO: 185 - protein, 16 aa, synthetic construct
EGYDLWSGYSSNWLDP

SEQ ID NO: 186 - protein, 9 aa, synthetic construct
DEGLTTGEF

SEQ ID NO: 187 - protein, 9 aa, synthetic construct
DEGLTTGES

SEQ ID NO: 188 - protein, 9 aa, synthetic construct
EEGLTTGEY

SEQ ID NO: 189 - protein, 9 aa, synthetic construct
DEGLTSGEY

SEQ ID NO: 190 - protein, 9 aa, synthetic construct
DFQLELRDY

SEQ ID NO: 191 - protein, 9 aa, synthetic construct
DFQLELRDF

SEQ ID NO: 192 - protein, 9 aa, synthetic construct
DHPKFGFDY

SEQ ID NO: 193 - protein, 12 aa, synthetic construct
GGVWNYGGSLDF

SEQ ID NO: 194 - protein, 12 aa, synthetic construct
GGVWKYGGSLDF

SEQ ID NO: 195 - protein, 12 aa, synthetic construct
GSVWNYGGSLDF

SEQ ID NO: 196 - protein, 12 aa, synthetic construct
GSVWNYGGSLDY

SEQ ID NO: 197 - protein, 12 aa, synthetic construct
GSVWKYGGSLDY

SEQ ID NO: 198 - protein, 12 aa, synthetic construct
GSVWRYGGSLDF

SEQ ID NO: 199 - protein, 11 aa, synthetic construct
GSSWHVYFFDF

SEQ ID NO: 200 - protein, 11 aa, synthetic construct
GSSWSLYYFDF

SEQ ID NO: 201 - protein, 11 aa, synthetic construct
GSSWYVYYFDY

SEQ ID NO: 202 - protein, 11 aa, synthetic construct
GSSWYVYYFDF

SEQ ID NO: 203 - protein, 7 aa, synthetic construct
EGLTTGE

SEQ ID NO: 204 - protein, 7 aa, synthetic construct
RWEPSFH

SEQ ID NO: 205 - protein, 10 aa, synthetic construct
LTYYYGAGSD

SEQ ID NO: 206 - protein, 7 aa, synthetic construct
FQLELPD

SEQ ID NO: 207 - protein, 7 aa, synthetic construct
HPKFGFD

SEQ ID NO: 208 - protein, 10 aa, synthetic construct
SVWKYGGSLD

SEQ ID NO: 209 - protein, 9 aa, synthetic construct
SSWNVYFFD

SEQ ID NO: 210 - protein, 14 aa, synthetic construct
GYDLWSGDSSNWLD

SEQ ID NO: 211 - protein, 14 aa, synthetic construct
GYDLWSGESSNWLD

SEQ ID NO: 212 - protein, 14 aa, synthetic construct
GYDLWSGHSSNWLD

SEQ ID NO: 213 - protein, 14 aa, synthetic construct
GYDLWSGYSSNWLD

SEQ ID NO: 214 - protein, 7 aa, synthetic construct
EGLTSGE

SEQ ID NO: 215 - protein, 7 aa, synthetic construct
FQLELRD

SEQ ID NO: 216 - protein, 10 aa, synthetic construct
GVWNYGGSLD

SEQ ID NO: 217 - protein, 10 aa, synthetic construct
GVWKYGGSLD

SEQ ID NO: 218 - protein, 10 aa, synthetic construct
SVWNYGGSLD

SEQ ID NO: 219 - protein, 10 aa, synthetic construct
SVWRYGGSLD

SEQ ID NO: 220 - protein, 9 aa, synthetic construct
SSWHVYFFD

SEQ ID NO: 221 - protein, 9 aa, synthetic construct
SSWSLYYFD

SEQ ID NO: 222 - protein, 9 aa, synthetic construct
SSWYVYYFD

SEQ ID NO: 223 - protein, 11 aa, synthetic construct
VKDEGLTTGEY

SEQ ID NO: 224 - protein, 11 aa, synthetic construct
AKERWEPSFHY

SEQ ID NO: 225 - protein, 14 aa, synthetic construct
AGTLTYYYGAGSDY

SEQ ID NO: 226 - protein, 11 aa, synthetic construct
VRDFQLELPDY

SEQ ID NO: 227 - protein, 11 aa, synthetic construct
TTDHPKFGFDF

SEQ ID NO: 228 - protein, 14 aa, synthetic construct
STGSVWKYGGSLDF

SEQ ID NO: 229 - protein, 13 aa, synthetic construct
TTGSSWNVYFFDF

SEQ ID NO: 230 - protein, 18 aa, synthetic construct
AREGYDLWSGDSSNWLDP

SEQ ID NO: 231 - protein, 18 aa, synthetic construct
AREGYDLWSGESSNWLDP

SEQ ID NO: 232 - protein, 18 aa, synthetic construct
AREGYDLWSGHSSNWLDP

SEQ ID NO: 233 - protein, 18 aa, synthetic construct
AREGYDLWSGYSSNWLDP

SEQ ID NO: 234 - protein, 11 aa, synthetic construct
AKDEGLTTGEY

SEQ ID NO: 235 - protein, 11 aa, synthetic construct
VKDEGLTTGEF

SEQ ID NO: 236 - protein, 11 aa, synthetic construct
VKDEGLTTGES

SEQ ID NO: 237 - protein, 11 aa, synthetic construct
AKEEGLTTGEY

SEQ ID NO: 238 - protein, 11 aa, synthetic construct
AKDEGLTSGEY

SEQ ID NO: 239 - protein, 11 aa, synthetic construct
VKEEGLTTGEY

SEQ ID NO: 240 - protein, 11 aa, synthetic construct
VRDFQLELRDY

SEQ ID NO: 241 - protein, 11 aa, synthetic construct
VSDFQLELPDY

SEQ ID NO: 242 - protein, 11 aa, synthetic construct
VRDFQLELRDF

SEQ ID NO: 243 - protein, 11 aa, synthetic construct
TTDHPKFGFDY

SEQ ID NO: 244 - protein, 14 aa, synthetic construct
TTGGVWNYGGSLDF

SEQ ID NO: 245 - protein, 14 aa, synthetic construct
ITGGVWNYGGSLDF

SEQ ID NO: 246 - protein, 14 aa, synthetic construct
TTGGVWKYGGSLDF

SEQ ID NO: 247 - protein, 14 aa, synthetic construct
ATGGVWKYGGSLDF

SEQ ID NO: 248 - protein, 14 aa, synthetic construct
TTGSVWKYGGSLDF

SEQ ID NO: 249 - protein, 14 aa, synthetic construct
STGSVWNYGGSLDF

SEQ ID NO: 250 - protein, 14 aa, synthetic construct
TTGSVWNYGGSLDY

SEQ ID NO: 251 - protein, 14 aa, synthetic construct
TTGSVWKYGGSLDY

SEQ ID NO: 252 - protein, 14 aa, synthetic construct
TTGSVWRYGGSLDF

SEQ ID NO: 253 - protein, 14 aa, synthetic construct
TTGSVWNYGGSLDF

SEQ ID NO: 254 - protein, 13 aa, synthetic construct
TTGSSWHVYFFDF

SEQ ID NO: 255 - protein, 13 aa, synthetic construct
TTGSSWSLYYFDF

SEQ ID NO: 256 - protein, 13 aa, synthetic construct
TTGSSWYVYYFDY

SEQ ID NO: 257 - protein, 13 aa, synthetic construct
TTGSSWYVYYFDF

SEQ ID NO: 258 - protein, 11 aa, synthetic construct
RASQDIRNDLA

SEQ ID NO: 259 - protein, 11 aa, synthetic construct
QASQGITNYLN

SEQ ID NO: 260 - protein, 11 aa, synthetic construct
RASQGIRNDLG

SEQ ID NO: 261 - protein, 11 aa, synthetic construct
RASQGIRDDLA

SEQ ID NO: 262 - protein, 17 aa, synthetic construct
KSSQSVFYSSNNKNYLA

SEQ ID NO: 263 - protein, 11 aa, synthetic construct
RASQGISRFLN

SEQ ID NO: 264 - protein, 11 aa, synthetic construct
RASQSISNYLN

SEQ ID NO: 265 - protein, 11 aa, synthetic construct
RASQGISSYLA

SEQ ID NO: 266 - protein, 11 aa, synthetic construct
RASQDIRNDLD

SEQ ID NO: 267 - protein, 11 aa, synthetic construct
RASQAIRNDLG

SEQ ID NO: 268 - protein, 11 aa, synthetic construct
RTSQGIRNDLA

SEQ ID NO: 269 - protein, 11 aa, synthetic construct
RASQGIRNDLA

SEQ ID NO: 270 - protein, 11 aa, synthetic construct
RASQGIRNNLG

SEQ ID NO: 271 - protein, 11 aa, synthetic construct
RASQAIRDDLA

SEQ ID NO: 272 - protein, 17 aa, synthetic construct
KSSQSVFYTSNSKHYLN

SEQ ID NO: 273 - protein, 17 aa, synthetic construct
KSSQSVLYSSNNKNYLA

SEQ ID NO: 274 - protein, 17 aa, synthetic construct
KSSQSVFYTSNNKNYLN

SEQ ID NO: 275 - protein, 17 aa, synthetic construct
KSSQSVFYTSSNKHYLN

SEQ ID NO: 276 - protein, 11 aa, synthetic construct
RASQSITRYLN

SEQ ID NO: 277 - protein, 11 aa, synthetic construct
RASQFITRYLN

SEQ ID NO: 278 - protein, 11 aa, synthetic construct
RASQGISRYLN

SEQ ID NO: 279 - protein, 11 aa, synthetic construct
RASQSITRSLN

SEQ ID NO: 280 - protein, 11 aa, synthetic construct
RASQSISRYLN

SEQ ID NO: 281 - protein, 11 aa, synthetic construct
RASQNISRYLN

SEQ ID NO: 282 - protein, 11 aa, synthetic construct
RASQIISRYLN

SEQ ID NO: 283 - protein, 11 aa, synthetic construct
RASQSMSSHLN

SEQ ID NO: 284 - protein, 11 aa, synthetic construct
RASQSISTYLN

SEQ ID NO: 285 - protein, 11 aa, synthetic construct
RASQSISSYLN

SEQ ID NO: 286 - protein, 7 aa, synthetic construct
SQDIRND

SEQ ID NO: 287 - protein, 7 aa, synthetic construct
SQGITNY

SEQ ID NO: 288 - protein, 7 aa, synthetic construct
SQGIRND

SEQ ID NO: 289 - protein, 7 aa, synthetic construct
SQGIRDD

SEQ ID NO: 290 - protein, 13 aa, synthetic construct
SQSVFYSSNNKNY

SEQ ID NO: 291 - protein, 7 aa, synthetic construct
SQGISRF

SEQ ID NO: 292 - protein, 7 aa, synthetic construct
SQSISNY

SEQ ID NO: 293 - protein, 7 aa, synthetic construct
SQGISSY

SEQ ID NO: 294 - protein, 7 aa, synthetic construct
SQAIRND

SEQ ID NO: 295 - protein, 7 aa, synthetic construct
SQGIRNN

SEQ ID NO: 296 - protein, 7 aa, synthetic construct
SQAIRDD

SEQ ID NO: 297 - protein, 13 aa, synthetic construct
SQSVFYTSNSKHY

SEQ ID NO: 298 - protein, 13 aa, synthetic construct
SQSVLYSSNNKNY

SEQ ID NO: 299 - protein, 13 aa, synthetic construct
SQSVFYTSNNKNY

SEQ ID NO: 300 - protein, 13 aa, synthetic construct
SQSVFYTSSNKHY

SEQ ID NO: 301 - protein, 7 aa, synthetic construct
SQSITRY

SEQ ID NO: 302 - protein, 7 aa, synthetic construct
SQFITRY

SEQ ID NO: 303 - protein, 7 aa, synthetic construct
SQGISRY

SEQ ID NO: 304 - protein, 7 aa, synthetic construct
SQSITRS

SEQ ID NO: 305 - protein, 7 aa, synthetic construct
SQSISRY

SEQ ID NO: 306 - protein, 7 aa, synthetic construct
SQNISRY

SEQ ID NO: 307 - protein, 7 aa, synthetic construct
SQIISRY

SEQ ID NO: 308 - protein, 7 aa, synthetic construct
SQSMSSH

SEQ ID NO: 309 - protein, 7 aa, synthetic construct
SQSISTY

SEQ ID NO: 310 - protein, 7 aa, synthetic construct
SQSISSY

SEQ ID NO: 311 - protein, 6 aa, synthetic construct
QDIRND

SEQ ID NO: 312 - protein, 6 aa, synthetic construct
QGITNY

SEQ ID NO: 313 - protein, 6 aa, synthetic construct
QGIRND

SEQ ID NO: 314 - protein, 6 aa, synthetic construct
QGIRDD

SEQ ID NO: 315 - protein, 12 aa, synthetic construct
QSVFYSSNNKNY

SEQ ID NO: 316 - protein, 6 aa, synthetic construct
QGISRF

SEQ ID NO: 317 - protein, 6 aa, synthetic construct
QSISNY

SEQ ID NO: 318 - protein, 6 aa, synthetic construct
QGISSY

SEQ ID NO: 319 - protein, 6 aa, synthetic construct
QAIRND

SEQ ID NO: 320 - protein, 6 aa, synthetic construct
QAIRDD

SEQ ID NO: 321 - protein, 12 aa, synthetic construct
QSVFYTSNSKHY

SEQ ID NO: 322 - protein, 12 aa, synthetic construct
QSVLYSSNNKNY

SEQ ID NO: 323 - protein, 12 aa, synthetic construct
QSVFYTSNNKNY

SEQ ID NO: 324 - protein, 12 aa, synthetic construct
QSVFYTSSNKHY

SEQ ID NO: 325 - protein, 6 aa, synthetic construct
QSITRY

SEQ ID NO: 326 - protein, 6 aa, synthetic construct
QFITRY

SEQ ID NO: 327 - protein, 6 aa, synthetic construct
QGISRY

SEQ ID NO: 328 - protein, 6 aa, synthetic construct
QSITRS

SEQ ID NO: 329 - protein, 6 aa, synthetic construct
QSISRY

SEQ ID NO: 330 - protein, 6 aa, synthetic construct
QNISRY

SEQ ID NO: 331 - protein, 6 aa, synthetic construct
QIISRY

SEQ ID NO: 332 - protein, 6 aa, synthetic construct
QSMSSH

SEQ ID NO: 333 - protein, 6 aa, synthetic construct
QSISTY

SEQ ID NO: 334 - protein, 6 aa, synthetic construct
QSISSY

SEQ ID NO: 335 - protein, 7 aa, synthetic construct
ASSSLQS

SEQ ID NO: 336 - protein, 7 aa, synthetic construct
DASNLET

SEQ ID NO: 337 - protein, 7 aa, synthetic construct
AASSLQS

SEQ ID NO: 338 - protein, 7 aa, synthetic construct
WASTRES

SEQ ID NO: 339 - protein, 7 aa, synthetic construct
AASTLQN

SEQ ID NO: 340 - protein, 7 aa, synthetic construct
AAYRLQT

SEQ ID NO: 341 - protein, 7 aa, synthetic construct
AASTLQS

SEQ ID NO: 342 - protein, 7 aa, synthetic construct
VVSSFQS

SEQ ID NO: 343 - protein, 7 aa, synthetic construct
ATSSLQS

SEQ ID NO: 344 - protein, 7 aa, synthetic construct
ATSNLQS

SEQ ID NO: 345 - protein, 7 aa, synthetic construct
AASSFQS

SEQ ID NO: 346 - protein, 7 aa, synthetic construct
AASSLKS

SEQ ID NO: 347 - protein, 7 aa, synthetic construct
ATSSFQS

SEQ ID NO: 348 - protein, 7 aa, synthetic construct
AVSSLQS

SEQ ID NO: 349 - protein, 7 aa, synthetic construct
ATSILQS

SEQ ID NO: 350 - protein, 7 aa, synthetic construct
AASSLQN

SEQ ID NO: 351 - protein, 7 aa, synthetic construct
AASILQS

SEQ ID NO: 352 - protein, 7 aa, synthetic construct
GASTLQS

SEQ ID NO: 353 - protein, 7 aa, synthetic construct
AASRLQS

SEQ ID NO: 354 - protein, 7 aa, synthetic construct
GASSLES

SEQ ID NO: 355 - protein, 7 aa, synthetic construct
GASSLQS

SEQ ID NO: 356 - protein, 7 aa, synthetic construct
AASRLES

SEQ ID NO: 357 - protein, 7 aa, synthetic construct
AASNLQS

SEQ ID NO: 358 - protein, 7 aa, synthetic construct
AASSLQS

SEQ ID NO: 359-367 - skipped (9 entries)

SEQ ID NO: 368 - protein, 9 aa, synthetic construct
LQHNNYPFT

SEQ ID NO: 369 - protein, 9 aa, synthetic construct
QQYENLPYT

SEQ ID NO: 370 - protein, 9 aa, synthetic construct
LQHNSYPWS

SEQ ID NO: 371 - protein, 9 aa, synthetic construct
LQHKSNPFT

SEQ ID NO: 372 - protein, 9 aa, synthetic construct
QQHYDIPRT

SEQ ID NO: 373 - protein, 9 aa, synthetic construct
QQTYSTPLT

SEQ ID NO: 374 - protein, 9 aa, synthetic construct
QQSYSSPLT

SEQ ID NO: 375 - protein, 9 aa, synthetic construct
QQLNSYPRT

SEQ ID NO: 376 - protein, 9 aa, synthetic construct
QQLNNYPRT

SEQ ID NO: 377 - protein, 9 aa, synthetic construct
QQLQSYPRT

SEQ ID NO: 378 - protein, 9 aa, synthetic construct
LQHNSYPFT

SEQ ID NO: 379 - protein, 9 aa, synthetic construct
LQHNTYPFS

SEQ ID NO: 380 - protein, 9 aa, synthetic construct
LQHSSYPFT

SEQ ID NO: 381 - protein, 9 aa, synthetic construct
LQHNTYPFT

SEQ ID NO: 382 - protein, 9 aa, synthetic construct
LQHNSNPFT

SEQ ID NO: 383 - protein, 9 aa, synthetic construct
LQHYSNPFT

SEQ ID NO: 384 - protein, 9 aa, synthetic construct
QQYYSIPRT

SEQ ID NO: 385 - protein, 9 aa, synthetic construct
QQHYSTPRT

SEQ ID NO: 386 - protein, 9 aa, synthetic construct
QQYYSTPRT

SEQ ID NO: 387 - protein, 9 aa, synthetic construct
QQSYSTPLT

SEQ ID NO: 388 - protein, 9 aa, synthetic construct
QQSYNTPLT

SEQ ID NO: 389 - protein, 9 aa, synthetic construct
QQSYSTPVT

SEQ ID NO: 390 - protein, 9 aa, synthetic construct
QQSYRTSLT

SEQ ID NO: 391 - protein, 9 aa, synthetic construct
QQSYGTPLT

SEQ ID NO: 392 - protein, 9 aa, synthetic construct
QQSYSTPFT

SEQ ID NO: 393 - protein, 9 aa, synthetic construct
QQSYRSPFT

SEQ ID NO: 394 - protein, 6 aa, synthetic construct
HNNYPF

SEQ ID NO: 395 - protein, 6 aa, synthetic construct
YENLPY

SEQ ID NO: 396 - protein, 6 aa, synthetic construct
HNSYPW

SEQ ID NO: 397 - protein, 6 aa, synthetic construct
HKSNPF

SEQ ID NO: 398 - protein, 6 aa, synthetic construct
HYDIPR

SEQ ID NO: 399 - protein, 6 aa, synthetic construct
TYSTPL

SEQ ID NO: 400 - protein, 6 aa, synthetic construct
SYSSPL

SEQ ID NO: 401 - protein, 6 aa, synthetic construct
LNSYPR

SEQ ID NO: 402 - protein, 6 aa, synthetic construct
LNNYPR

SEQ ID NO: 403 - protein, 6 aa, synthetic construct
LQSYPR

SEQ ID NO: 404 - protein, 6 aa, synthetic construct
HNSYPF

SEQ ID NO: 405 - protein, 6 aa, synthetic construct
HNTYPF

SEQ ID NO: 406 - protein, 6 aa, synthetic construct
HSSYPF

SEQ ID NO: 407 - protein, 6 aa, synthetic construct
HNSNPF

SEQ ID NO: 408 - protein, 6 aa, synthetic construct
HYSNPF

SEQ ID NO: 409 - protein, 6 aa, synthetic construct
YYSIPR

SEQ ID NO: 410 - protein, 6 aa, synthetic construct
HYSTPR

SEQ ID NO: 411 - protein, 6 aa, synthetic construct
YYSTPR

SEQ ID NO: 412 - protein, 6 aa, synthetic construct
SYSTPL

SEQ ID NO: 413 - protein, 6 aa, synthetic construct
SYNTPL

SEQ ID NO: 414 - protein, 6 aa, synthetic construct
SYSTPV

SEQ ID NO: 415 - protein, 6 aa, synthetic construct
SYRTSL

SEQ ID NO: 416 - protein, 6 aa, synthetic construct
SYGTPL

SEQ ID NO: 417 - protein, 6 aa, synthetic construct
SYSTPF

SEQ ID NO: 418 - protein, 6 aa, synthetic construct
SYRSPF

SEQ ID NO: 419 - protein, 118 aa, synthetic construct
EVQVVESGGGLVQPGGSLRLSCAASGFTFSNYAMTWVRQSPGKGLEWVSLISGSGGLTKYADSVKGRFTISRDNSKKTLFLQMNSLRAEDTAAYYCVKDEGLTTGEYWGQGTQVTVSS

SEQ ID NO: 420 - protein, 118 aa, synthetic construct
KVQLVESGGGLVQPGGSLRLSCAASGFRFSSYAMNWVRQAPGKGLEWVSLISGSGGLTKYADSVKGRFTISRDNSRNTLYLQMISLRAEDTAVYFCAKERWEPSFHYWGQGTLVTVSS

SEQ ID NO: 421 - protein, 120 aa, synthetic construct
EVQVVESGGGLIQPGGSLRISCAASGFPVSSNYMSWVRQAPGKGLDWVSIIYSRDSTHYADSVKGRFTMSRDNSKNTVYLQMNSLRAEDTAVYYCAGTLTYYYGAGSDYWGQGTLVTVSS

SEQ ID NO: 422 - protein, 118 aa, synthetic construct
EVQLVESGGGLVQPGGSLRLSCAASGFTFSNYWMHWVRQAPGKGLVWVSRINSDGISTRYADSVKGRFTISRDNAKNTLYLQMKSLRAEDTAVYYCVRDFQLELPDYWGQGTLVTVSS

SEQ ID NO: 423 - protein, 120 aa, synthetic construct
EVQLVESGGGFVKPGGSLRLSCAASGFTFSNAWMSWVRQAPGKGLEWVGRIKSNSYGGTTDYAAPAIGRFTISRDGSKSTLYLQMNSLKTEDTAVYYCTTDHPKFGFDFWGQGTLVTVSS

SEQ ID NO: 424 - protein, 123 aa, synthetic construct
EVQLVESGGGLVTPGGSLRISCAASGFTFNNAWMTWVRQAPGKGLEWVGRIKSISYGGTTDSAAPVKGRFTISRDDSENTLYLQMSSLKTEDTAVYYCSTGSVWKYGGSLDFWGQGTLVTVSS

SEQ ID NO: 425 - protein, 122 aa, synthetic construct
EVQLVESGGGLVKPGGSLRLSCAASGFTFSYAWMSWVRQAPGRGLEWVGRIKSETDGGTTDYAAPVKGRFTISRDHSENTLFLQMNSLKTEDTAVYYCTTGSSWNVYFFDFWGQGTLVTVSS

SEQ ID NO: 426 - protein, 125 aa, synthetic construct
QVQLVQSGAEVKKPGASVKVSCKASGYTFTNYDINWVRQATGQGLEWMGWMNPHSGQTGYAQKFQGRVTMTRNTAISTAYMELSSLRSEDTAVYYCAREGYDLWSGDSSNWLDPWGQGTLVTVSA

SEQ ID NO: 427 - protein, 125 aa, synthetic construct
QVQLVQSGAEVKKPGASVKVSCKASGYTFTNYDINWVRQATGQGLEWMGWMNPHSGQTGYAQKFQGRVTMTRNTAISTAYMELSSLRSEDTAVYYCAREGYDLWSGESSNWLDPWGQGTLVTVSA

SEQ ID NO: 428 - protein, 125 aa, synthetic construct
QVQLVQSGAEVKKPGASVKVSCKASGYTFTNYDINWVRQATGQGLEWMGWMNPHSGQTGYAQKFQGRVTMTRNTAISTAYMELSSLRSEDTAVYYCAREGYDLWSGHSSNWLDPWGQGTLVTVSA

SEQ ID NO: 429 - protein, 125 aa, synthetic construct
QVQLVQSGAEVKKPGASVKVSCKASGYTFTNYDINWVRQATGQGLEWMGWMNPHSGQTGYAQKFQGRVTMTRNTAISTAYMELSSLRSEDTAVYYCAREGYDLWSGYSSNWLDPWGQGTLVTVSA

SEQ ID NO: 430 - protein, 118 aa, synthetic construct
EVQVVESGGGLVQPGGSLRLSCAASGFTFSSYAMYWVRQSPGKGLEWVSLITGSGGSTHYADSVKGRFTISRDNSKKTLFLQMNSLRAEDTAAYYCAKDEGLTTGEYWGQGTQVTVSS

SEQ ID NO: 431 - protein, 118 aa, synthetic construct
EVQVVESGGGLVQPGGSLRLSCAASGFTFRNYAMTWVRQSPGKGLEWVSLISGSGGITKYADSVKGRFTISRDNSKKTLFLQMNSLRAEDTAAYYCVKDEGLTTGEYWGQGTQVTVSS

SEQ ID NO: 432 - protein, 118 aa, synthetic construct
EVQVVESGGGLVQPGGSLRLSCAASGFTFSSYAMTWVRQSPGKGLEWVSLISGSGGITHYADSVKGRFTISRDNSKKTLFLQMNSLRAEDTAAYYCVKDEGLTTGEFWGQGTQVTVSS

SEQ ID NO: 433 - protein, 118 aa, synthetic construct
EVQVVESGGGLVQPGGSLRLSCAASGFTFSSYAMTWVRQSPGKGLEWVSLISGSGGVTHYADSVKGRFTISRDNSKKTLFLQMNSLRAEDTAAYYCVKDEGLTTGEFWGQGTQVTVSS

SEQ ID NO: 434 - protein, 118 aa, synthetic construct
EVQVVESGGGLVQPGGSLRLSCAASGFTFSSYAMSWVRQSPGKGLEWVSLISGSGGITHYADSVKGRFTISRDNSKKTLFLQMNSLRAEDTAAYYCVKDEGLTTGEYWGQGTQVTVSS

SEQ ID NO: 435 - protein, 118 aa, synthetic construct
EVQVVESGGGLVQPGGSLRLSCAASGFTFSSYAMSWVRQSPGKGLEWVSLISGSGAITHYADSVKGRFTISRDNSKKTLFLQMNSLRAEDTAAYYCVKDEGLTTGEYWGQGTQVTVSS

SEQ ID NO: 436 - protein, 118 aa, synthetic construct
EVQVVESGGGLVQPGGSLRLSCAASGFTFSSYAMTWVRQSPGKGLEWVSLISGSGGVTHYADSVKGRFTISRDNSKKTLFLQMNSLRAEDTAAYYCVKDEGLTTGESWGQGTQVTVSS

SEQ ID NO: 437 - protein, 118 aa, synthetic construct
EVQVVESGGGLVQPGGSLRLSCAASGFTFSSYAMTWVRQSPGKGLEWVSLISGSGGITHYADSVKGRFTISRDNSKKTLFLQMNSLRAEDTAAYYCVKDEGLTTGESWGQGTQVTVSS

SEQ ID NO: 438 - protein, 118 aa, synthetic construct
EVQVVESGGGLVQPGGSLRLSCAASGFTFSSYAMTWVRQSPGKGLEWVSLISGSGGLTKYADSVKGRFTISRDNSKKTLFLQMNSLRAEDTAAYYCAKEEGLTTGEYWGQGTQVTVSS

SEQ ID NO: 439 - protein, 118 aa, synthetic construct
EVQVVESGGGLVQPGGSLRLSCAASGFTFNTYAMTWVRQSPGKGLEWVSLISGSGGLTKYADSVKGRFTISRDNSKKTLFLQMNSLRAEDTAAYYCAKEEGLTTGEYWGQGTQVTVSS

SEQ ID NO: 440 - protein, 118 aa, synthetic construct
EVQVVESGGGLVQPGGSLRLSCAASGFTFSSYAMTWVRQSPGKGLEWVSLISGSGGLTNYADSVKGRFTISRDNSKKTLFLQMNSLRAEDTAAYYCAKDEGLTSGEYWGQGTQVTVSS

SEQ ID NO: 441 - protein, 118 aa, synthetic construct
EVQVVESGGGLVQPGGSLRLSCAASGFTFSSYAMTWVRQSPGKGLEWVSLISGSGGSTHYADSVKGRFTISRDNSKKTLFLQMNSLRAEDTAAYYCAKDEGLTTGEYWGQGTQVTVSS

SEQ ID NO: 442 - protein, 118 aa, synthetic construct
EVQVVESGGGLVQPGGSLRLSCAASGFTFSSHAMTWVRQSPGKGLEWVSLISGSGGLTKYADSVRGRFTISRDNSKKTLFLQMNSLRAEDTAAYYCVKEEGLTTGEYWGQGTQVTVSS

SEQ ID NO: 443 - protein, 118 aa, synthetic construct
EVQVVESGGGLVQPGGSLRLSCAASGFTFSSYAMTWVRQSPGKGLEWVSLISGSGGLTKYADSVKGRFTISRDNSKKTLFLQMNSLRAEDTAAYYCAKDEGLTTGEYWGQGTQVTVSS

SEQ ID NO: 444 - protein, 118 aa, synthetic construct
EVQVVESGGGLVQPGGSLRLSCAASGFTFSSYAMTWVRQSPGKGLEWVSLISGSGSLTKYADSVKGRFTISRDNSKKTLFLQMNSLRAEDTAAYYCVKEEGLTTGEYWGQGTQVTVSS

SEQ ID NO: 445 - protein, 118 aa, synthetic construct
EVQVVESGGGLVQPGGSLRLSCAASGFTFSSYAMTWVRQSPGKGLEWVSLISGSGGLTKYADSVKGRFTISRDNSKKTLFLQMNSLRAEDTAAYYCVKDEGLTTGEYWGQGTQVTVSS

SEQ ID NO: 446 - protein, 118 aa, synthetic construct
EVQVVESGGGLVQPGGSLRLSCAASGFTFGNYAMTWVRQSPGKGLEWVSLISGSGGLTKYADSVKGRFTISRDNSKKTLFLQMNSLRAEDTAAYYCVKDEGLTTGEYWGQGTQVTVSS

SEQ ID NO: 447 - protein, 118 aa, synthetic construct
EVQVVESGGGLVQPGGSLRLSCAASGFTFTNYAMTWVRQSPGKGLEWVSLISGGGGLTYYADSVKGRFTISRDNSKKTLFLQMNSLRAEDTAAYYCVKDEGLTTGEYWGQGTQVTVSS

SEQ ID NO: 448 - protein, 118 aa, synthetic construct
EVQVVESGGGLVQPGGSLRLSCAASGFTFSIYAMTWVRQSPGKGLEWVSLISGSGGLTKYADSVKGRFTISRDNSKKTLFLQMNSLRAEDTAAYYCVKDEGLTTGEYWGQGTQVTVSS

SEQ ID NO: 449 - protein, 118 aa, synthetic construct
EVQVVESGGGLVQPGGSLRLSCAASGFTFSSYAMTWVRQSPGKGLEWVSLISGSGGLTYYADSVKGRFTISRDNSKKTLFLQMNSLRAEDTAAYYCVKDEGLTTGEYWGQGTQVTVSS

SEQ ID NO: 450 - protein, 118 aa, synthetic construct
EVQVVESGGGLVQPGGSLRLSCAASGFTFSSHAMTWVRQSPGKGLEWVSLISGSGGLTYYADSVKGRFTISRDNSKKTLFLQMNSLRAEDTAAYYCVKDEGLTTGEYWGQGTQVTVSS

SEQ ID NO: 451 - protein, 118 aa, synthetic construct
EVQVVESGGGLVQPGGSLRLSCAASGFTFSNYAMTWVRQSPGKGLEWVSLISGGGGLTYYADSVKGRFTISRDNSKKTLFLQMNSLRAEDTAAYYCVKDEGLTTGEYWGQGTQVTVSS

SEQ ID NO: 452 - protein, 118 aa, synthetic construct
EVQVVESGGGLVQPGGSLRLSCAASGFTFSSYAMTWVRQSPGKGLEWVSLISGSGGLTKYADSVKGRFTISRDNSKKTLFLQMNSLRAEDTAAYYCVKEEGLTTGEYWGQGTQVTVSS

SEQ ID NO: 453 - protein, 118 aa, synthetic construct
EVQVVESGGGLVQPGGSLRLSCAASGFTFSSYAMTWVRQSPGKGLEWVSLISGSGGITKYADSVKGRFTISRDNSKKTLFLQMNSLRAEDTAAYYCAKEEGLTTGEYWGQGTQVTVSS

SEQ ID NO: 454 - protein, 118 aa, synthetic construct
EVQLVESGGGLVQPGGSLRLSCAASGFRFSSYAMNWVRQAPGKGLEWVSLISGSGGLTKYADSVKGRFTISRDNSRNTLYLQMISLRAEDTAVYFCAKERWEPSFHYWGQGTLVTVSS

SEQ ID NO: 455 - protein, 118 aa, synthetic construct
EVQLVESGGGLVQPGGSLRLSCAASGFTFRSYWMHWVRQAPGKGLVWVSRIDNNGINTRYADSVKGRFTISRDNAKNTLYLQMKSLRAEDTAVYYCVRDFQLELRDYWGQGTLVTVSS

SEQ ID NO: 456 - protein, 118 aa, synthetic construct
EVQLVESGGGLVQPGGSLRLSCAASGFTFSNYWMHWVRQAPGKGLVWVSRISSDGISTRYADSVKGRFTISRDNAKNTLYLQMKSLRAEDTAVYYCVRDFQLELPDYWGQGTLVTVSS

SEQ ID NO: 457 - protein, 118 aa, synthetic construct
EVQLVESGGGLVQPGGSLRLSCAASGLTFSNYWMHWVRQAPGKGLVWVSRINSDGISTRYADSVKGRFTISRDNAKNTLYLQMKSLRAEDTAVYYCVSDFQLELPDYWGQGTLVTVSS

SEQ ID NO: 458 - protein, 118 aa, synthetic construct
EVQLVESGGGLVQPGGSLRLSCAASGFTFSNYWMHWVRQAPGKGLVWVSRINNDGISTRYADSVKGRFTISRDNAKNTLYLQMKSLRAEDTAVYYCVRDFQLELPDYWGQGTLVTVSS

SEQ ID NO: 459 - protein, 118 aa, synthetic construct
EVQLVESGGGLVQPGGSLRLSCAASGFTFSSFWMHWVRQAPGKGLVWVSRINSDGFSTRYADSVKGRFTISRDNAKNTLYLQMKSLRAEDTAVYYCVRDFQLELRDFWGQGTLVTVSS

SEQ ID NO: 460 - protein, 118 aa, synthetic construct
EVQLVESGGGLVQPGGSLRLSCAASGFTFSSFWMHWVRQAPGKGLVWVSRINSDGFSTRYADSVKGRFTISRDNAKNTLYLQMKSLRAEDTAVYYCVRDFQLELRDYWGQGTLVTVSS

SEQ ID NO: 461 - protein, 118 aa, synthetic construct
EVQLVESGGGLVQPGGSLRLSCAASGFTFSSYWMHWVRQAPGKGLVWVSRINSDGISTRYADSVKGRFTISRDNAKNTLYLQMKSLRAEDTAVYYCVRDFQLELRDYWGQGTLVTVSS

SEQ ID NO: 462 - protein, 120 aa, synthetic construct
EVQLVESGGGFVKPGGSLRLSCAASGFTFHNAWMSWVRQAPGKGLEWVGRIKGVTAGGTADYAAPVKGRFTISRDGSKSTLYLQMNSLKTEDTAVYYCTTDHPKFGFDFWGQGTLVTVSS

SEQ ID NO: 463 - protein, 120 aa, synthetic construct
EVQLVESGGGFVKPGGSLRLSCAASGFTFNNAWMSWVRQAPGKGLEWVGRVKSVTAGGTTDYAAPVKGRFTISRDGSKSTLYLQMNSLKTEDTAVYYCTTDHPKFGFDFWGQGTLVTVSS

SEQ ID NO: 464 - protein, 120 aa, synthetic construct
EVQLVESGGGFVKPGGSLRLSCAASGFTFSNAWMSWVRQAPGKGLEWVGRIKSKTDGGTTDYAAPVKGRFTISRDGSKSTLYLQMNSLKTEDTAVYYCTTDHPKFGFDYWGQGTLVTVSS

SEQ ID NO: 465 - protein, 120 aa, synthetic construct
EVQLVESGGGFVKPGGSLRLSCAASGFTFNSAWMSWVRQAPGKGLEWVGRIKSVTTGGTTDYAAPVKGRFTISRDGSKSTLYLQMNSLKTEDTAVYYCTTDHPKFGFDFWGQGTLVTVSS

SEQ ID NO: 466 - protein, 120 aa, synthetic construct
EVQLVESGGGFVKPGGSLRLSCAASGFTFNNAWMSWVRQAPGKGLEWVGRIKSVTAGGTTDYAAPVKGRFTISRDGSKSTLYLQMNSLKTEDTAVYYCTTDHPKFGFDYWGQGTLVTVSS

SEQ ID NO: 467 - protein, 120 aa, synthetic construct
EVQLVESGGGFVKPGGSLRLSCAASGFTFNNAWMSWVRQAPGKGLEWVGRIKSVTAGGTTDFPAPVKGRFTISRDGSKSTLYLQMNSLKTEDTAVYYCTTDHPKFGFDFWGQGTLVTVSS

SEQ ID NO: 468 - protein, 123 aa, synthetic construct
EVQLVESGGGLVTPGGSLRISCAASGFIFNNAWMRWVRQAPGKGLEWVGHIKSETDGGTTDYAAPVKGRFTISRDDSENTLYLQMSSLKTEDTAVYYCTTGGVWNYGGSLDFWGQGTLVTVSS

SEQ ID NO: 469 - protein, 123 aa, synthetic construct
EVQLVESGGGLVTPGGSLRISCAASGFIFNNAWMRWVRQAPGKGLEWVGHIKSETDGGTTDYAAPVKGRFTISRDDSENTLYLQMSSLKTEDTAVYYCITGGVWNYGGSLDFWGQGTLVTVSS

SEQ ID NO: 470 - protein, 123 aa, synthetic construct
EVQLVESGGGLVTPGGSLRISCAASGFIFNNAWMRWVRQAPGKGLEWVGHIKSETDGGTTDYAAPVKGRFTISRDDSENTLYLQMSSLKTEDTAVYYCTTGGVWKYGGSLDFWGQGTLVTVSS

SEQ ID NO: 471 - protein, 123 aa, synthetic construct
EVQLVESGGGLVTPGGSLRISCAASGFIFNNAWMRWVRQAPGKGLEWVGHIKSETDGGTTDYAAPVKGRFTISRDDSENTLYLQMSSLKTEDTAVYYCATGGVWKYGGSLDFWGQGTLVTVSS

SEQ ID NO: 472 - protein, 123 aa, synthetic construct
EVQLVESGGGLVTPGGSLRISCAASGFTFNYAWMTWVRQAPGKGLEWVGRIKRKTDGGTTDYAAPVQGRFTISRDDSENTLYLQMSSLKTEDTAVYYCTTGSVWKYGGSLDFWGQGTLVTVSS

SEQ ID NO: 473 - protein, 123 aa, synthetic construct
EVQLVESGGGLVTPGGSLRISCAASGFTFINAWMNWVRQAPGKGLEWVGRIKSLTDGGTTDYAAPVKGRFTISRDDSENTLYLQMSSLKTEDTAVYYCSTGSVWNYGGSLDFWGQGTLVTVSS

SEQ ID NO: 474 - protein, 123 aa, synthetic construct
EVQLVESGGGLVTPGGSLRISCAASGFTFSNAWMNWVRQAPGKGLEWVGRIKRKTDGGTREYAAPVKGRFTISRDDSENTLYLQMSSLKTEDTAVYYCTTGSVWKYGGSLDFWGQGTLVTVSS

SEQ ID NO: 475 - protein, 123 aa, synthetic construct
EVQLVESGGGLVTPGGSLRISCAASGFTFSNAWMSWVRQAPGKGLEWVGRIKRNTDGGTTDYAAPVKGRFTISRDDSENTLYLQMSSLKTEDTAVYYCTTGSVWKYGGSLDFWGQGTLVTVSS

SEQ ID NO: 476 - protein, 123 aa, synthetic construct
EVQLVESGGGLVTPGGSLRISCAASGFTFSNAWMSWVRQAPGKGLEWVGRIKTNTDGGTTDYAAPVKGRFTISRDDSENTLYLQMSSLKTEDTAVYYCTTGSVWNYGGSLDYWGQGTLVTVSS

SEQ ID NO: 477 - protein, 123 aa, synthetic construct
EVQLVESGGGLVTPGGSLRISCAASGFTFSNAWMSWVRQAPGKGLEWVGRIKSKTYGGTTDYDAPVKGRFTISRDDSENTLYLQMSSLKTEDTAVYYCTTGSVWKYGGSLDYWGQGTLVTVSS

SEQ ID NO: 478 - protein, 123 aa, synthetic construct
EVQLVESGGGLVTPGGSLRISCAASGFTFSNAWMSWVRQAPGKGLEWVGRIKSQTYGGTTDYAAPVKGRFTISRDDSENTLYLQMSSLKTEDTAVYYCTTGSVWKYGGSLDYWGQGTLVTVSS

SEQ ID NO: 479 - protein, 123 aa, synthetic construct
EVQLVESGGGLVTPGGSLRISCAASGFTFSNAWMSWVRQAPGKGLEWVGRIKSKTDGGTTDYAAPVKGRFTISRDDSENTLYLQMSSLKTEDTAVYYCTTGSVWRYGGSLDFWGQGTLVTVSS

SEQ ID NO: 480 - protein, 123 aa, synthetic construct
EVQLVESGGGLVTPGGSLRISCAASGFTFSNAWMNWVRQAPGKGLEWVGRIKSKTDGGTTDYAAPVKGRFTISRDDSENTLYLQMSSLKTEDTAVYYCTTGSVWNYGGSLDFWGQGTLVTVSS

SEQ ID NO: 481 - protein, 123 aa, synthetic construct
EVQLVESGGGLVTPGGSLRISCAASGFTFSNAWMSWVRQAPGKGLEWVGRIKSKTDGGTTDYAAPVKGRFTISRDDSENTLYLQMSSLKTEDTAVYYCTTGSVWKYGGSLDFWGQGTLVTVSS

SEQ ID NO: 482 - protein, 123 aa, synthetic construct
EVQLVESGGGLVTPGGSLRISCAASGFTFSNAWMTWVRQAPGKGLEWVGRIKSKTYGGTTDYAAPVKGRFTISRDDSENTLYLQMSSLKTEDTAVYYCTTGSVWNYGGSLDFWGQGTLVTVSS

SEQ ID NO: 483 - protein, 123 aa, synthetic construct
EVQLVESGGGLVTPGGSLRISCAASGFTFINAWMNWVRQAPGKGLEWVGRIKRKTDGGTRDYAAPVKGRFTISRDDSENTLYLQMSSLKTEDTAVYYCTTGSVWKYGGSLDFWGQGTLVTVSS

SEQ ID NO: 484 - protein, 122 aa, synthetic construct
EVQLVESGGGLVKPGGSLRLSCAASGFTFSNAWMSWVRQAPGRGLEWVGRIKRETDGGTTDYASPVKGRFTISRDHSENTLFLQMNSLKTEDTAVYYCTTGSSWNVYFFDFWGQGTLVTVSS

SEQ ID NO: 485 - protein, 122 aa, synthetic construct
EVQLVESGGGLVKPGGSLRLSCAASGFTFSTAWMSWVRQAPGRGLEWVGRIKRKSDGGTTDYAAPVKGRFTISRDHSENTLFLQMNSLKTEDTAVYYCTTGSSWHVYFFDFWGQGTLVTVSS

SEQ ID NO: 486 - protein, 122 aa, synthetic construct
EVQLVESGGGLVKPGGSLRLSCAASGFIFSNAWMSWVRQAPGRGLEWVGRIKSKTYGGTTDYAAPVKGRFTISRDHSENTLFLQMNSLKTEDTAVYYCTTGSSWSLYYFDFWGQGTLVTVSS

SEQ ID NO: 487 - protein, 122 aa, synthetic construct
EVQLVESGGGLVKPGGSLRLSCAASGFIFSNAWMSWVRQAPGRGLEWVGRIKSKTDGGTTDYAAPVKGRFTISRDHSENTLFLQMNSLKTEDTAVYYCTTGSSWYVYYFDYWGQGTLVTVSS

SEQ ID NO: 488 - protein, 122 aa, synthetic construct
EVQLVESGGGLVKPGGSLRLSCAASGFIFSNAWMSWVRQAPGRGLEWVGRIKSEIDGGTTDYAAPVKGRFTISRDHSENTLFLQMNSLKTEDTAVYYCTTGSSWYVYYFDFWGQGTLVTVSS

SEQ ID NO: 489 - protein, 107 aa, synthetic construct
DIQMTQSPSSLSASVGDRVTITCRASQDIRNDLAWYQQKPGKAPKRLIYASSSLQSGVPSRFSGSGSGTEFTLTISSLQPEDFATYYCLQHNNYPFTFGPGTKVDIK

SEQ ID NO: 490 - protein, 107 aa, synthetic construct
DIQMTQSPSSLSASVGDSVIITCQASQGITNYLNWYQQKPGKAPNLLIYDASNLETGVPSRFSGSGSGTYFTFTISSLQPEDIATYFCQQYENLPYTFGQGTKLEIK

SEQ ID NO: 491 - protein, 107 aa, synthetic construct
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPWSFGQGTKVEIK

SEQ ID NO: 492 - protein, 107 aa, synthetic construct
DIQMTQSPSSLSASVGDRVTITCRASQGIRDDLAWYQQKAGKAPKRLIFAASSLQSGVPSRFSGSGSGTEFTLTISGLQPEDFANYYCLQHKSNPFTFGQGTKLEIK

SEQ ID NO: 493 - protein, 113 aa, synthetic construct
DIVMTQSPDSLAVSLGERATIDCKSSQSVFYSSNNKNYLAWFQQKPGQPPKLLIYWASTRESGVPDRFSGSGSGTDFTLTISSLQAEDVAVYYCQQHYDIPRTFGQGTKVEIK

SEQ ID NO: 494 - protein, 107 aa, synthetic construct
DIQMTQSPSSLSASVGDRVTFTCRASQGISRFLNWYQQKPGKAPKLLIYAASTLQNGVPSRFSGSGSGTDFTLTISSLQPEDFATYFCQQTYSTPLTFGGGTKVEIY

SEQ ID NO: 495 - protein, 107 aa, synthetic construct
DIQMTQSPSSLSASVGDSVTITCRASQSISNYLNWYHQKPGKAPKLLIYAAYRLQTGVPSRFSGSGSGTDFTLTISSLQPEDFATYYCQQSYSSPLTFGGGTKVEIK

SEQ ID NO: 496 - protein, 107 aa, synthetic construct
DIQLTQSPSFLSASVGDRVTITCRASQGISSYLAWYQQKPGKAPKLLIYAASTLQSGFRSRFSGSGSGTEFTLTISSLQPEDFASYYCQQLNSYPRTFGQGTKVEIK

SEQ ID NO: 497 - protein, 107 aa, synthetic construct
DIQLTQSPSFLSASVGDRVTITCRASQGISSYLAWYQQKPGKAPKLLIYAASTLQSGFRSRFSGSGSGTEFTLTISSLQPEDFASYYCQQLNNYPRTFGQGTKVEIK

SEQ ID NO: 498 - protein, 107 aa, synthetic construct
DIQLTQSPSFLSASVGDRVTITCRASQGISSYLAWYQQKPGKAPKLLIYAASTLQSGFRSRFSGSGSGTEFTLTISSLQPEDFASYYCQQLQSYPRTFGQGTKVEIK

SEQ ID NO: 499 - protein, 107 aa, synthetic construct
DIQMTQSPSSLSASVGDRVTITCRASQGIRNDLGWYQQKPGKAPKRLIYVVSSFQSGVPSRFSGSGSGTEFTLTISSLQPEDFATYYCLQHNNYPFTFGPGTKVDIK

SEQ ID NO: 500 - protein, 107 aa, synthetic construct
DIQMTQSPSSLSASVGDRVTITCRASQDIRNDLDWYQQKPGKAPKRLIYATSSLQSGVPSRFSGSGSGTEFTLTISSLQPEDFATYYCLQHNSYPFTFGPGTKVDIK

SEQ ID NO: 501 - protein, 107 aa, synthetic construct
DIQMTQSPSSLSASVGDRVTITCRASQAIRNDLGWYQQKPGKAPKRLIYATSNLQSGVPSRFSGSGSGTEFTLTISSLQPEDFATYYCLQHNSYPFTFGPGTKVDIK

SEQ ID NO: 502 - protein, 107 aa, synthetic construct
DIQMTQSPSSLSASVGDRVTITCRASQGIRNDLGWYQQKPGKAPKRLIYATSSLQSGVPSRFSGSGSGTEFTLTISSLQPEDFATYYCLQHNSYPFTFGPGTKVDIK

SEQ ID NO: 503 - protein, 107 aa, synthetic construct
DIQMTQSPSSLSASVGDRVTITCRTSQGIRNDLAWYQQKPGKAPKRLIYAASSLQSGVPSRFSGSGSGTEFTLTISSLQPEDFATYYCLQHNNYPFTFGPGTKVDIK

SEQ ID NO: 504 - protein, 107 aa, synthetic construct
DIQMTQSPSSLSASVGDRVTITCRASQGIRNDLAWYQQKPGKAPKRLIYAASSFQSGVPSRFSGSGSGTEFTLTISSLQPEDFATYYCLQHNSYPFTFGPGTKVDIK

SEQ ID NO: 505 - protein, 107 aa, synthetic construct
DIQMTQSPSSLSASVGDRVTITCRASQGIRNDLGWYQQKPGKAPKRLIYAASSLKSGVPSRFSGSGSGTEFTLTISSLQPEDFATYYCLQHNSYPFTFGPGTKVDIK

SEQ ID NO: 506 - protein, 107 aa, synthetic construct
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPFTFGPGTKVDIK

SEQ ID NO: 507 - protein, 107 aa, synthetic construct
DIQMTQSPSSLSASVGDRVTITCRASQGIRNDLGWYQQKPGKAPKRLIYATSSFQSGVPSRFSGSGSGTEFTLTISSLQPEDFATYYCLQHNSYPFTFGPGTKVDIK

SEQ ID NO: 508 - protein, 107 aa, synthetic construct
DIQMTQSPSSLSASVGDRVTITCRASQGIRNDLGWYQQKPGKAPKRLIYAASSFQSGVPSRFSGSGSGTEFTLTISSLQPEDFATYYCLQHNSYPFTFGPGTKVDIK

SEQ ID NO: 509 - protein, 107 aa, synthetic construct
DIQMTQSPSSLSASVGDRVTITCRASQGIRNNLGWYQQKPGKAPKRLIYAASSLQSGVPSRFSGSGSGTEFTLTISSLQPEDFATYYCLQHNSYPFTFGPGTKVDIK

SEQ ID NO: 510 - protein, 107 aa, synthetic construct
DIQMTQSPSSLSASVGDRVTITCRASQGIRNDLGWYQQKPGKAPKRLIYAASSLQSGVPSRFSGSGSGTEFTLTISSLQPEDFATYYCLQHNTYPFSFGPGTKVDIK

SEQ ID NO: 511 - protein, 107 aa, synthetic construct
DIQMTQSPSSLSASVGDRVTITCRASQGIRNDLGWYQQKPGKAPKRLIYAASSLQSGVPSRFSGSGSGTEFTLTISSLQPEDFATYYCLQHSSYPFTFGPGTKVDIK

SEQ ID NO: 512 - protein, 107 aa, synthetic construct
DIQMTQSPSSLSASVGDRVTITCRASQGIRNDLGWYQQKPGKAPKRLIYAASSLQSGVPSRFSGSGSGTEFTLTISSLQPEDFATYYCLQHNNYPFTFGPGTKVDIK

SEQ ID NO: 513 - protein, 107 aa, synthetic construct
DIQMTQSPSSLSASVGDRVTITCRASQGIRNDLGWYQQKPGKAPKRLIYAVSSLQSGVPSRFSGSGSGTEFTLTISSLQPEDFATYYCLQHNSYPFTFGPGTKVDIK

SEQ ID NO: 514 - protein, 107 aa, synthetic construct
DIQMTQSPSSLSASVGDRVTITCRASQGIRNDLGWYQQKPGKAPKRLIYAASSLQSGVPSRFSGSGSGTEFTLTISSLQPEDFATYYCLQHNTYPFTFGPGTKVDIK

SEQ ID NO: 515 - protein, 107 aa, synthetic construct
DIQMTQSPSSLSASVGDRVTITCRASQGIRNDLGWYQQKPGKAPKRLIYASSSLQSGVPSRFSGSGSGTEFTLTISSLQPEDFATYYCLQHNNYPFTFGPGTKVDIK

SEQ ID NO: 516 - protein, 107 aa, synthetic construct
DIQMTQSPSSLSASVGDRVTITCRASQGIRNDLAWYQQKAGKAPKRLIFATSILQSGVPSRFSGSGSGTEFTLTISGLQPEDFANYYCLQHNSNPFTFGQGTKLEIK

SEQ ID NO: 517 - protein, 107 aa, synthetic construct
DIQMTQSPSSLSASVGDRVTITCRASQGIRNDLGWYQQKAGKAPKRLIFAASSLQSGVPSRFSGSGSGTEFTLTISGLQPEDFANYYCLQHKSNPFTFGQGTKLEIK

SEQ ID NO: 518 - protein, 107 aa, synthetic construct
DIQMTQSPSSLSASVGDRVTITCRASQAIRDDLAWYQQKAGKAPKRLIFAASSLQNGVPSRFSGSGSGTEFTLTISGLQPEDFANYYCLQHKSNPFTFGQGTKLEIK

SEQ ID NO: 519 - protein, 107 aa, synthetic construct
DIQMTQSPSSLSASVGDRVTITCRASQGIRNDLAWYQQKAGKAPKRLIFAASILQSGVPSRFSGSGSGTEFTLTISGLQPEDFANYYCLQHNSNPFTFGQGTKLEIK

SEQ ID NO: 520 - protein, 107 aa, synthetic construct
DIQMTQSPSSLSASVGDRVTITCRASQGIRNDLAWYQQKAGKAPKRLIFAASILQSGVPSRFSGSGSGTEFTLTISGLQPEDFANYYCLQHKSNPFTFGQGTKLEIK

SEQ ID NO: 521 - protein, 107 aa, synthetic construct
DIQMTQSPSSLSASVGDRVTITCRASQGIRNDLAWYQQKAGKAPKRLIFAASSLQSGVPSRFSGSGSGTEFTLTISGLQPEDFANYYCLQHNSNPFTFGQGTKLEIK

SEQ ID NO: 522 - protein, 107 aa, synthetic construct
DIQMTQSPSSLSASVGDRVTITCRASQGIRNDLAWYQQKAGKAPKRLIFAASILQSGVPSRFSGSGSGTEFTLTISGLQPEDFANYYCLQHYSNPFTFGQGTKLEIK

SEQ ID NO: 523 - protein, 113 aa, synthetic construct
DIVMTQSPDSLAVSLGERATIDCKSSQSVFYTSNSKHYLNWFQQKPGQPPKLLIYWASTRESGVPDRFSGSGSGTDFTLTISSLQAEDVAVYYCQQYYSIPRTFGQGTKVEIK

SEQ ID NO: 524 - protein, 113 aa, synthetic construct
DIVMTQSPDSLAVSLGERATIDCKSSQSVLYSSNNKNYLAWFQQKPGQPPKLLIYWASTRESGVPDRFSGSGSGTDFTLTISSLQAEDVAVYYCQQHYSTPRTFGQGTKVEIK

SEQ ID NO: 525 - protein, 113 aa, synthetic construct
DIVMTQSPDSLAVSLGERATIDCKSSQSVFYTSNSKHYLNWFQQKPGQPPKLLIYWASTRESGVPDRFSGSGSGTDFTLTISSLQAEDVAVYYCQQYYSTPRTFGQGTKVEIK

SEQ ID NO: 526 - protein, 113 aa, synthetic construct
DIVMTQSPDSLAVSLGERATIDCKSSQSVFYTSNNKNYLNWFQQKPGQPPKLLIYWASTRESGVPDRFSGSGSGTDFTLTISSLQAEDVAVYYCQQYYSTPRTFGQGTKVEIK

SEQ ID NO: 527 - protein, 113 aa, synthetic construct
DIVMTQSPDSLAVSLGERATIDCKSSQSVFYTSSNKHYLNWFQQKPGQPPKLLIYWASTRESGVPDRFSGSGSGTDFTLTISSLQAEDVAVYYCQQYYSTPRTFGQGTKVEIK

SEQ ID NO: 528 - protein, 107 aa, synthetic construct
DIQMTQSPSSLSASVGDRVTFTCRASQSITRYLNWYQQKPGKAPKLLIYAASSLQSGVPSRFSGSGSGTDFTLTISSLQPEDFATYFCQQSYSTPLTFGGGTKVEIY

SEQ ID NO: 529 - protein, 107 aa, synthetic construct
DIQMTQSPSSLSASVGDRVTFTCRASQFITRYLNWYQQKPGKAPKLLIYAASSLQSGVPSRFSGSGSGTDFTLTISSLQPEDFATYFCQQSYSTPLTFGGGTKVEIY

SEQ ID NO: 530 - protein, 107 aa, synthetic construct
DIQMTQSPSSLSASVGDRVTFTCRASQSITRYLNWYQQKPGKAPKLLIYAASSLQSGVPSRFSGSGSGTDFTLTISSLQPEDFATYFCQQSYNTPLTFGGGTKVEIY

SEQ ID NO: 531 - protein, 107 aa, synthetic construct
DIQMTQSPSSLSASVGDRVTFTCRASQGISRYLNWYQQKPGKAPKLLIYGASTLQSGVPSRFSGSGSGTDFTLTISSLQPEDFATYFCQQSYSTPLTFGGGTKVEIY

SEQ ID NO: 532 - protein, 107 aa, synthetic construct
DIQMTQSPSSLSASVGDRVTFTCRASQSITRSLNWYQQKPGKAPKLLIYAASRLQSGVPSRFSGSGSGTDFTLTISSLQPEDFATYFCQQSYSSPLTFGGGTKVEIY

SEQ ID NO: 533 - protein, 107 aa, synthetic construct
DIQMTQSPSSLSASVGDRVTFTCRASQSISRYLNWYQQKPGKAPKLLIYAASSLQSGVPSRFSGSGSGTDFTLTISSLQPEDFATYFCQQSYSTPVTFGGGTKVEIY

SEQ ID NO: 534 - protein, 107 aa, synthetic construct
DIQMTQSPSSLSASVGDRVTFTCRASQSISRYLNWYQQKPGKAPKLLIYAASSLQNGVPSRFSGSGSGTDFTLTISSLQPEDFATYFCQQSYSTPVTFGGGTKVEIY

SEQ ID NO: 535 - protein, 107 aa, synthetic construct
DIQMTQSPSSLSASVGDRVTFTCRASQSISRYLNWYQQKPGKAPKLLIYGASSLESGVPSRFSGSGSGTDFTLTISSLQPEDFATYFCQQSYRTSLTFGGGTKVEIY

SEQ ID NO: 536 - protein, 107 aa, synthetic construct
DIQMTQSPSSLSASVGDRVTFTCRASQNISRYLNWYQQKPGKAPKLLIYAASSLQSGVPSRFSGSGSGTDFTLTISSLQPEDFATYFCQQSYSTPLTFGGGTKVEIY

SEQ ID NO: 537 - protein, 107 aa, synthetic construct
DIQMTQSPSSLSASVGDRVTFTCRASQIISRYLNWYQQKPGKAPKLLIYAASSLQSGVPSRFSGSGSGTDFTLTISSLQPEDFATYFCQQSYGTPLTFGGGTKVEIY

SEQ ID NO: 538 - protein, 107 aa, synthetic construct
DIQMTQSPSSLSASVGDRVTFTCRASQSISRYLNWYQQKPGKAPKLLIYGASSLQSGVPSRFSGSGSGTDFTLTISSLQPEDFATYFCQQSYSTPLTFGGGTKVEIY

SEQ ID NO: 539 - protein, 107 aa, synthetic construct
DIQMTQSPSSLSASVGDRVTFTCRASQSISRYLNWYQQKPGKAPKLLIYAASSLQSGVPSRFSGSGSGTDFTLTISSLQPEDFATYFCQQSYSTPLTFGGGTKVEIY

SEQ ID NO: 540 - protein, 107 aa, synthetic construct
DIQMTQSPSSLSASVGDSVTITCRASQSMSSHLNWYHQKPGKAPKLLIYAASRLESGVPSRFSGSGSGTDFTLTISSLQPEDFATYYCQQSYSSPLTFGGGTKVEIK

SEQ ID NO: 541 - protein, 107 aa, synthetic construct
DIQMTQSPSSLSASVGDSVTITCRASQSISRYLNWYHQKPGKAPKLLIYAASSLQSGVPSRFSGSGSGTDFTLTISSLQPEDFATYYCQQSYSSPLTFGGGTKVEIK

SEQ ID NO: 542 - protein, 107 aa, synthetic construct
DIQMTQSPSSLSASVGDSVTITCRASQSISTYLNWYHQKPGKAPKLLIYAASSLQSGVPSRFSGSGSGTDFTLTISSLQPEDFATYYCQQSYSSPLTFGGGTKVEIK

SEQ ID NO: 543 - protein, 107 aa, synthetic construct
DIQMTQSPSSLSASVGDSVTITCRASQSISSYLNWYHQKPGKAPKLLIYAASNLQSGVPSRFSGSGSGTDFTLTISSLQPEDFATYYCQQSYSTPFTFGGGTKVEIK

SEQ ID NO: 544 - protein, 107 aa, synthetic construct
DIQMTQSPSSLSASVGDSVTITCRASQSISSYLNWYHQKPGKAPKLLIYAASSLQSGVPSRFSGSGSGTDFTLTISSLQPEDFATYYCQQSYRSPFTFGGGTKVEIK

SEQ ID NO: 545 - protein, 16 aa, synthetic construct
  VARIANT 9: note = D, E, H, or Y
EGYDLWSGXSSNWLDP

SEQ ID NO: 546 - protein, 14 aa, synthetic construct
  VARIANT 8: note = D, E, H, or Y
GYDLWSGXSSNWLD

SEQ ID NO: 547 - protein, 18 aa, synthetic construct
  VARIANT 11: note = D, E, H, or Y
AREGYDLWSGXSSNWLDP

SEQ ID NO: 548 - protein, 9 aa, synthetic construct
  VARIANT 4: note = N or Q
  VARIANT 5: note = S or N
QQLXXYPRT

SEQ ID NO: 549 - protein, 6 aa, synthetic construct
  VARIANT 2: note = N or Q
  VARIANT 3: note = S or N
LXXYPR

SEQ ID NO: 550 - protein, 9 aa, synthetic construct
  VARIANT 4: note = N or Q
  VARIANT 5: note = S or N
QQLXXYPRT

SEQ ID NO: 551 - skipped

SEQ ID NO: 552 - protein, 17 aa, synthetic construct
  VARIANT 3: note = S or T
  VARIANT 5: note = S or G
  VARIANT 7: note = G, A, or S
  VARIANT 8: note = L, S, I, or V
  VARIANT 10: note = K, H, N, or Y
  VARIANT 16: note = K or R
LIXGXGXXTXYADSVXG

SEQ ID NO: 553 - protein, 9 aa, synthetic construct
  VARIANT 1: note = D or E
  VARIANT 6: note = T or S
  VARIANT 9: note = Y, F, or S
XEGLTXGEX

SEQ ID NO: 554 - protein, 11 aa, synthetic construct
  VARIANT 2: note = A or T
  VARIANT 5: note = D, G, or A
  VARIANT 9: note = D or N
  VARIANT 11: note = A, G, or D
RXSQXIRNXLX

SEQ ID NO: 555 - skipped

SEQ ID NO: 556 - protein, 9 aa, synthetic construct
  VARIANT 4: note = N or S
  VARIANT 5: note = N, S, or T
  VARIANT 9: note = T or S
LQHXXYPFX

SEQ ID NO: 557 - protein, 7 aa, synthetic construct
  VARIANT 5: note = S, R, N, G, or T
  VARIANT 6: note = N, S, T, or I
  VARIANT 7: note = Y or H
GFTFXXX

SEQ ID NO: 558 - skipped

SEQ ID NO: 559 - protein, 7 aa, synthetic construct
  VARIANT 5: note = T or S
EGLTXGE

SEQ ID NO: 560 - protein, 7 aa, synthetic construct
  VARIANT 3: note = D, G, or A
  VARIANT 7: note = D or N
SQXIRNX

SEQ ID NO: 561 - skipped

SEQ ID NO: 562 - protein, 6 aa, synthetic construct
  VARIANT 2: note = N or S
  VARIANT 3: note = N, S, or T
HXXYPF

SEQ ID NO: 563 - protein, 8 aa, synthetic construct
  VARIANT 5: note = S, R, N, G, or T
  VARIANT 6: note = N, S, T, or I
  VARIANT 7: note = Y or H
GFTFXXXA

SEQ ID NO: 564 - protein, 8 aa, synthetic construct
  VARIANT 2: note = S or T
  VARIANT 4: note = S or G
  VARIANT 6: note = G, A, or S
  VARIANT 7: note = L, S, I, or V
IXGXGXXT

SEQ ID NO: 565 - protein, 11 aa, synthetic construct
  VARIANT 1: note = V or A
  VARIANT 3: note = D or E
  VARIANT 8: note = T or S
  VARIANT 11: note = Y, F, or S
XKXEGLTXGEX

SEQ ID NO: 566 - protein, 6 aa, synthetic construct
  VARIANT 2: note = D, G, or A
  VARIANT 6: note = D or N
QXIRNX

SEQ ID NO: 567 - skipped

SEQ ID NO: 568 - protein, 9 aa, synthetic construct
  VARIANT 4: note = N or S
  VARIANT 5: note = N, S, or T
  VARIANT 9: note = T or S
LQHXXYPFX

SEQ ID NO: 569 - skipped

SEQ ID NO: 570 - protein, 17 aa, synthetic construct
  VARIANT 3: note = N, D, or S
  VARIANT 4: note = S or N
  VARIANT 5: note = D or N
  VARIANT 7: note = I or F
  VARIANT 8: note = S or N
RIXXXGXXTRYADSVKG

SEQ ID NO: 571 - protein, 9 aa, synthetic construct
  VARIANT 7: note = P or R
  VARIANT 9: note = Y or F
DFQLELXDX

SEQ ID NO: 572 - protein, 11 aa, synthetic construct
  VARIANT 5: note = G or A
  VARIANT 8: note = D or N
  VARIANT 11: note = A or G
RASQXIRXDLX

SEQ ID NO: 573 - protein, 7 aa, synthetic construct
  VARIANT 2: note = A or T
  VARIANT 4: note = S or I
  VARIANT 7: note = S or N
AXSXLQX

SEQ ID NO: 574 - protein, 9 aa, synthetic construct
  VARIANT 4: note = K, N, or Y
LQHXSNPFT

SEQ ID NO: 575-576 - skipped (2 entries)

SEQ ID NO: 577 - protein, 7 aa, synthetic construct
  VARIANT 6: note = P or R
FQLELXD

SEQ ID NO: 578 - protein, 7 aa, synthetic construct
  VARIANT 3: note = G or A
  VARIANT 6: note = D or N
SQXIRXD

SEQ ID NO: 579 - skipped

SEQ ID NO: 580 - protein, 6 aa, synthetic construct
  VARIANT 2: note = K, N, or Y
HXSNPF

SEQ ID NO: 581 - protein, 8 aa, synthetic construct
  VARIANT 2: note = F or L
  VARIANT 5: note = S or R
  VARIANT 6: note = N or S
  VARIANT 7: note = Y or F
GXTFXXXW

SEQ ID NO: 582 - skipped

SEQ ID NO: 583 - protein, 11 aa, synthetic construct
  VARIANT 2: note = R or S
  VARIANT 9: note = P or R
  VARIANT 11: note = Y or F
VXDFQLELXDX

SEQ ID NO: 584 - protein, 6 aa, synthetic construct
  VARIANT 2: note = G or A
  VARIANT 5: note = D or N
QXIRXD

SEQ ID NO: 585 - skipped

SEQ ID NO: 586 - protein, 9 aa, synthetic construct
  VARIANT 4: note = K, N, or Y
LQHXSNPFT

SEQ ID NO: 587 - protein, 5 aa, synthetic construct
  VARIANT 1: note = N or S
XAWMS

SEQ ID NO: 588 - protein, 19 aa, synthetic construct
  VARIANT 2: note = I or V
  VARIANT 4: note = S or G
  VARIANT 5: note = N, V, or K
  VARIANT 6: note = S or T
  VARIANT 7: note = Y, A, D, or T
  VARIANT 11: note = T or A
  VARIANT 13: note = Y or F
  VARIANT 14: note = A or P
  VARIANT 17: note = A or V
  VARIANT 18: note = I or K
RXKXXXXGGTXDXXAPXXG

SEQ ID NO: 589 - protein, 9 aa, synthetic construct
  VARIANT 9: note = F or Y
DHPKFGFDX

SEQ ID NO: 590 - protein, 17 aa, synthetic construct
  VARIANT 7: note = F or L
  VARIANT 9: note = S or T
  VARIANT 11: note = N or S
  VARIANT 12: note = N or S
  VARIANT 14: note = N or H
  VARIANT 17: note = A or N
KSSQSVXYXSXXKXYLX

SEQ ID NO: 591 - protein, 9 aa, synthetic construct
  VARIANT 3: note = H or Y
  VARIANT 5: note = D or S
  VARIANT 6: note = I or T
QQXYXXPRT

SEQ ID NO: 592 - protein, 7 aa, synthetic construct
  VARIANT 5: note = S, H, or N
  VARIANT 6: note = N or S
GFTFXXA

SEQ ID NO: 593 - protein, 8 aa, synthetic construct
  VARIANT 2: note = S or G
  VARIANT 3: note = N, V, or K
  VARIANT 4: note = S or T
  VARIANT 5: note = Y, A, D, or T
KXXXXGGT

SEQ ID NO: 594 - protein, 13 aa, synthetic construct
  VARIANT 5: note = F or L
  VARIANT 7: note = S or T
  VARIANT 9: note = N or S
  VARIANT 10: note = N or S
  VARIANT 12: note = N or H
SQSVXYXSXXKXY

SEQ ID NO: 595 - skipped

SEQ ID NO: 596 - protein, 8 aa, synthetic construct
  VARIANT 5: note = S, H, or N
  VARIANT 6: note = N or S
GFTFXXAW

SEQ ID NO: 597 - protein, 10 aa, synthetic construct
  VARIANT 1: note = I or V
  VARIANT 3: note = S or G
  VARIANT 4: note = N, V, or K
  VARIANT 5: note = S or T
  VARIANT 6: note = Y, A, D, or T
  VARIANT 10: note = T or A
XKXXXXGGTX

SEQ ID NO: 598 - protein, 11 aa, synthetic construct
  VARIANT 11: note = F or Y
TTDHPKFGFDX

SEQ ID NO: 599 - protein, 12 aa, synthetic construct
  VARIANT 4: note = F or L
  VARIANT 6: note = S or T
  VARIANT 8: note = N or S
  VARIANT 9: note = N or S
  VARIANT 11: note = N or H
QSVXYXSXXKXY

SEQ ID NO: 600 - protein, 9 aa, synthetic construct
  VARIANT 3: note = H or Y
  VARIANT 5: note = D or S
  VARIANT 6: note = I or T
QQXYXXPRT

SEQ ID NO: 601 - skipped

SEQ ID NO: 602 - protein, 19 aa, synthetic construct
  VARIANT 1: note = R or H
  VARIANT 4: note = S, R, or T
  VARIANT 5: note = I, E, K, L, N, or Q
  VARIANT 6: note = S or T
  VARIANT 7: note = Y or D
  VARIANT 11: note = T or R
  VARIANT 12: note = D or E
  VARIANT 13: note = S or Y
  VARIANT 14: note = A or D
  VARIANT 18: note = K or Q
XIKXXXXGGTXXXXAPVXG

SEQ ID NO: 603 - protein, 12 aa, synthetic construct
  VARIANT 2: note = S or G
  VARIANT 5: note = K, N, or R
  VARIANT 12: note = F or Y
GXVWXYGGSLDX

SEQ ID NO: 604 - protein, 11 aa, synthetic construct
  VARIANT 5: note = G, S, F, N, or I
  VARIANT 7: note = S or T
  VARIANT 9: note = F, Y, or S
RASQXIXRXLN

SEQ ID NO: 605 - skipped

SEQ ID NO: 606 - protein, 9 aa, synthetic construct
  VARIANT 3: note = T or S
  VARIANT 5: note = S, N, R, or G
  VARIANT 6: note = T or S
  VARIANT 7: note = P or S
  VARIANT 8: note = L or V
QQXYXXXXT

SEQ ID NO: 607 - protein, 7 aa, synthetic construct
  VARIANT 3: note = T or I
  VARIANT 5: note = N, I, or S
  VARIANT 6: note = N, or Y
GFXFXXA

SEQ ID NO: 608 - protein, 8 aa, synthetic construct
  VARIANT 2: note = S, R, or T
  VARIANT 3: note = I, E, K, L, N, or Q
  VARIANT 4: note = S or T
  VARIANT 5: note = Y or D
KXXXXGGT

SEQ ID NO: 609 - protein, 10 aa, synthetic construct
  VARIANT 1: note = S or G
  VARIANT 4: note = K, N, or R
XVWXYGGSLD

SEQ ID NO: 610 - protein, 7 aa, synthetic construct
  VARIANT 3: note = G, S, F, N, or I
  VARIANT 5: note = S or T
  VARIANT 7: note = F, Y, or S
SQXIXRX

SEQ ID NO: 611-612 - skipped (2 entries)

SEQ ID NO: 613 - protein, 8 aa, synthetic construct
  VARIANT 3: note = T or I
  VARIANT 5: note = N, I, or S
  VARIANT 6: note = N, or Y
GFXFXXAW

SEQ ID NO: 614 - protein, 10 aa, synthetic construct
  VARIANT 3: note = S, R, or T
  VARIANT 4: note = I, E, K, L, N, or Q
  VARIANT 5: note = S or T
  VARIANT 6: note = Y or D
  VARIANT 10: note = T or R
IKXXXXGGTX

SEQ ID NO: 615 - protein, 14 aa, synthetic construct
  VARIANT 1: note = S, T, I, or A
  VARIANT 4: note = S or G
  VARIANT 7: note = K, N, or R
  VARIANT 14: note = F or Y
XTGXVWXYGGSLDX

SEQ ID NO: 616-617 - skipped (2 entries)

SEQ ID NO: 618 - protein, 9 aa, synthetic construct
  VARIANT 3: note = T or S
  VARIANT 5: note = S, N, R, or G
  VARIANT 6: note = T or S
  VARIANT 7: note = P or S
  VARIANT 8: note = L or V
QQXYXXXXT

SEQ ID NO: 619 - protein, 5 aa, synthetic construct
  VARIANT 1: note = Y, N, or T
XAWMS

SEQ ID NO: 620 - protein, 19 aa, synthetic construct
  VARIANT 4: note = S or R
  VARIANT 5: note = E or K
  VARIANT 6: note = T, S, or I
  VARIANT 7: note = D or Y
  VARIANT 15: note = A or S
RIKXXXXGGTTDYAXPVKG

SEQ ID NO: 621 - protein, 11 aa, synthetic construct
  VARIANT 5: note = N, H, S or Y
  VARIANT 6: note = V or L
  VARIANT 8: note = F or Y
  VARIANT 11: note = F or Y
GSSWXXYXFDX

SEQ ID NO: 622 - protein, 11 aa, synthetic construct
  VARIANT 6: note = I or M
  VARIANT 8: note = N, S, R or T
  VARIANT 9: note = Y or H
RASQSXSXXLN

SEQ ID NO: 623 - skipped

SEQ ID NO: 624 - protein, 9 aa, synthetic construct
  VARIANT 5: note = S or R
  VARIANT 6: note = S or T
  VARIANT 8: note = L or F
QQSYXXPXT

SEQ ID NO: 625 - protein, 7 aa, synthetic construct
  VARIANT 3: note = T or I
  VARIANT 6: note = Y, N, or T
GFXFSXA

SEQ ID NO: 626 - protein, 8 aa, synthetic construct
  VARIANT 2: note = S or R
  VARIANT 3: note = E or K
  VARIANT 4: note = T, S, or I
  VARIANT 5: note = D or Y
KXXXXGGT

SEQ ID NO: 627 - protein, 9 aa, synthetic construct
  VARIANT 4: note = N, H, S or Y
  VARIANT 5: note = V or L
  VARIANT 7: note = F or Y
SSWXXYXFD

SEQ ID NO: 628 - protein, 7 aa, synthetic construct
  VARIANT 4: note = I or M
  VARIANT 6: note = N, S, R or T
  VARIANT 7: note = Y or H
SQSXSXX

SEQ ID NO: 629-630 - skipped (2 entries)

SEQ ID NO: 631 - protein, 8 aa, synthetic construct
  VARIANT 3: note = T or I
  VARIANT 6: note = Y, N, or T
GFXFSXAW

SEQ ID NO: 632 - protein, 10 aa, synthetic construct
  VARIANT 3: note = S or R
  VARIANT 4: note = E or K
  VARIANT 5: note = T, S, or I
  VARIANT 6: note = D or Y
IKXXXXGGTT

SEQ ID NO: 633 - protein, 13 aa, synthetic construct
  VARIANT 7: note = N, H, S or Y
  VARIANT 8: note = V or L
  VARIANT 10: note = F or Y
  VARIANT 13: note = F or Y
TTGSSWXXYXFDX

SEQ ID NO: 634-635 - skipped (2 entries)

SEQ ID NO: 636 - protein, 9 aa, synthetic construct
  VARIANT 5: note = S or R
  VARIANT 6: note = S or T
  VARIANT 8: note = L or F
QQSYXXPXT

SEQ ID NO: 637 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 638 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 639 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 640 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 641 - protein, 325 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 642 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 643 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 644 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 645 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 646 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 647 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 648 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 649 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 650 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 651 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 652 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 653 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 654 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 655 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 656 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 657 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 658 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 659 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 660 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 661 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 662 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 663 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 664 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 665 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 666 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 667 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 668 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 669 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 670 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 671 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 672 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 673 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 674 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 675 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLG

SEQ ID NO: 676 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLG

SEQ ID NO: 677 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLG

SEQ ID NO: 678 - protein, 325 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLYITREPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 679 - protein, 325 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSPG

SEQ ID NO: 680 - protein, 325 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVDHHDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHSHYTQKSLSLSPG

SEQ ID NO: 681 - protein, 326 aa, synthetic construct; identical to SEQ ID NO: 639

SEQ ID NO: 682 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 683 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 684 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 685 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 686 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 687 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 688 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPG

SEQ ID NO: 689 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 690 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 691 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 692 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 693 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 694 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 695 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPG

SEQ ID NO: 696 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 697 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 698 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 699 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 700 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 701 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 702 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPG

SEQ ID NO: 703 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 704 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 705 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 706 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 707 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 708 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 709 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 710 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 711 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 712 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 713 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 714 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 715 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 716 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 717 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 718 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 719 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 720 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 721 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 722 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 723 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 724 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 725 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 726 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 727 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 728 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 729 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 730 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 731 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 732 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYASTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 733 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 734 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 735 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 736 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 737 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPG

SEQ ID NO: 738 - protein, 107 aa, Homo sapiens [sequence omitted]

SEQ ID NO: 739 - protein, 183 aa, synthetic construct
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 740 - protein, 6 aa, synthetic construct
SGNWWS

SEQ ID NO: 741 - protein, 7 aa, synthetic construct
SGGYYWS

SEQ ID NO: 742 - protein, 8 aa, synthetic construct
GGSISSGN

SEQ ID NO: 743 - protein, 7 aa, synthetic construct
GFTFDSA

SEQ ID NO: 744 - protein, 9 aa, synthetic construct
GGSISSGGY

SEQ ID NO: 745 - protein, 9 aa, synthetic construct
GGSISSGNW

SEQ ID NO: 746 - protein, 8 aa, synthetic construct
GFTFDSAW

SEQ ID NO: 747 - protein, 10 aa, synthetic construct
GGSISSGGYY

SEQ ID NO: 748 - protein, 19 aa, synthetic construct
RFKSETDGGTKDFGAHVRG

SEQ ID NO: 749 - protein, 16 aa, synthetic construct
EIHHSGRTNYNPSLKS

SEQ ID NO: 750 - protein, 19 aa, synthetic construct
RIKRKTDGGTTDYAAPVKG

SEQ ID NO: 751 - protein, 19 aa, synthetic construct
RIKSKNEGGTIDYAAPVKG

SEQ ID NO: 752 - protein, 17 aa, synthetic construct
WIHPNTGKTGYAQKFQG

SEQ ID NO: 753 - protein, 16 aa, synthetic construct
HIYYSGSTKYNPSLKS

SEQ ID NO: 754 - protein, 5 aa, synthetic construct
HHSGR

SEQ ID NO: 755 - protein, 8 aa, synthetic construct
KSKNEGGT

SEQ ID NO: 756 - protein, 6 aa, synthetic construct
HPNTGK

SEQ ID NO: 757 - protein, 5 aa, synthetic construct
YYSGS

SEQ ID NO: 758 - protein, 10 aa, synthetic construct
FKSETDGGTK

SEQ ID NO: 759 - protein, 7 aa, synthetic construct
IHHSGRT

SEQ ID NO: 760 - protein, 10 aa, synthetic construct
IKSKNEGGTI

SEQ ID NO: 761 - protein, 8 aa, synthetic construct
IHPNTGKT

SEQ ID NO: 762 - protein, 7 aa, synthetic construct
IYYSGST

SEQ ID NO: 763 - protein, 9 aa, synthetic construct
GGTAVPFDF

SEQ ID NO: 764 - protein, 10 aa, synthetic construct
DRQGSFAFDI

SEQ ID NO: 765 - protein, 10 aa, synthetic construct
DRPWGSFFDY

SEQ ID NO: 766 - protein, 12 aa, synthetic construct
DFLWFGELPFDY

SEQ ID NO: 767 - protein, 16 aa, synthetic construct
ESYDLWSGHSFNWFDP

SEQ ID NO: 768 - protein, 12 aa, synthetic construct
DNWGSGWYYMDV

SEQ ID NO: 769 - protein, 7 aa, synthetic construct
GTAVPFD

SEQ ID NO: 770 - protein, 8 aa, synthetic construct
RQGSFAFD

SEQ ID NO: 771 - protein, 8 aa, synthetic construct
RPWGSFFD

SEQ ID NO: 772 - protein, 10 aa, synthetic construct
FLWFGELPFD

SEQ ID NO: 773 - protein, 14 aa, synthetic construct
SYDLWSGHSFNWFD

SEQ ID NO: 774 - protein, 10 aa, synthetic construct
NWGSGWYYMD

SEQ ID NO: 775 - protein, 11 aa, synthetic construct
TTGGTAVPFDF

SEQ ID NO: 776 - protein, 12 aa, synthetic construct
ARDRQGSFAFDI

SEQ ID NO: 777 - protein, 12 aa, synthetic construct
TLDRPWGSFFDY

SEQ ID NO: 778 - protein, 14 aa, synthetic construct
NTDFLWFGELPFDY

SEQ ID NO: 779 - protein, 18 aa, synthetic construct
ARESYDLWSGHSFNWFDP

SEQ ID NO: 780 - protein, 14 aa, synthetic construct
ARDNWGSGWYYMDV

SEQ ID NO: 781 - protein, 17 aa, synthetic construct
KSSQSLLYSSNNKNYLA

SEQ ID NO: 782 - protein, 11 aa, synthetic construct
RASQSISTWLV

SEQ ID NO: 783 - protein, 17 aa, synthetic construct
KSSQSVLYSSNNKNYFA

SEQ ID NO: 784 - protein, 11 aa, synthetic construct
RASQGISNHLA

SEQ ID NO: 785 - protein, 11 aa, synthetic construct
RASQGITSYLA

SEQ ID NO: 786 - protein, 11 aa, synthetic construct
RASQSVSNYFA

SEQ ID NO: 787 - protein, 13 aa, synthetic construct
SQSLLYSSNNKNY

SEQ ID NO: 788 - protein, 7 aa, synthetic construct
SQSISTW

SEQ ID NO: 789 - protein, 7 aa, synthetic construct
SQGISNH

SEQ ID NO: 790 - protein, 7 aa, synthetic construct
SQGITSY

SEQ ID NO: 791 - protein, 7 aa, synthetic construct
SQSVSNY

SEQ ID NO: 792 - protein, 12 aa, synthetic construct
QSLLYSSNNKNY

SEQ ID NO: 793 - protein, 6 aa, synthetic construct
QSISTW

SEQ ID NO: 794 - protein, 6 aa, synthetic construct
QGISNH

SEQ ID NO: 795 - protein, 6 aa, synthetic construct
QGITSY

SEQ ID NO: 796 - protein, 6 aa, synthetic construct
QSVSNY

SEQ ID NO: 797 - protein, 7 aa, synthetic construct
WTSTRES

SEQ ID NO: 798 - protein, 7 aa, synthetic construct
KASSLES

SEQ ID NO: 799 - protein, 7 aa, synthetic construct
RASTRES

SEQ ID NO: 800 - protein, 7 aa, synthetic construct
GATTLQS

SEQ ID NO: 801 - protein, 7 aa, synthetic construct
DASNRAT

SEQ ID NO: 802-805 - skipped (4 entries)

SEQ ID NO: 806 - protein, 9 aa, synthetic construct
LQYSNTPYS

SEQ ID NO: 807 - protein, 9 aa, synthetic construct
QQHISYPYT

SEQ ID NO: 808 - protein, 9 aa, synthetic construct
QQYYTTPLT

SEQ ID NO: 809 - protein, 9 aa, synthetic construct
QKLNSAPLT

SEQ ID NO: 810 - protein, 9 aa, synthetic construct
QQLQSYPYT

SEQ ID NO: 811 - protein, 9 aa, synthetic construct
QHRSNWPLT

SEQ ID NO: 812 - protein, 6 aa, synthetic construct
YSNTPY

SEQ ID NO: 813 - protein, 6 aa, synthetic construct
HISYPY

SEQ ID NO: 814 - protein, 6 aa, synthetic construct
YYTTPL

SEQ ID NO: 815 - protein, 6 aa, synthetic construct
LNSAPL

SEQ ID NO: 816 - protein, 6 aa, synthetic construct
LQSYPY

SEQ ID NO: 817 - protein, 6 aa, synthetic construct
RSNWPL

SEQ ID NO: 818 - protein, 120 aa, synthetic construct
EVQLVESGGGLVKPGGSLRLSCAASGFTFSNAWMSWVRQAPGKGLEWVGRFKSETDGGTKDFGAHVRGRFIISKDASKNTLYLQMNRLKTEDTAVYYCTTGGTAVPFDFWGLGTLVTVSS

SEQ ID NO: 819 - protein, 119 aa, synthetic construct
QVQLQESGPGLVKPSGTLSLTCAVSGGSISSGNWWSWVRQPPGSGLEWIGEIHHSGRTNYNPSLKSRVVISVDKSKNHFSLKLSSVTAADTAVYYCARDRQGSFAFDIWGQGTMVTVSS

SEQ ID NO: 820 - protein, 121 aa, synthetic construct
EVQLVESGGGLVKPGGSLRLSCTASGFTFDSAWMSWVRQAPGKGLEWVGRIKRKTDGGTTDYAAPVKGRFTISRDDSTKTLYLQMNSLKTEDTAVYYCTLDRPWGSFFDYWGQGTLVTVSS

SEQ ID NO: 821 - protein, 123 aa, synthetic construct
EVQLVESGGGLVKPGGSLRLSCAASGFTFSNAWMSWVRQAPGKGLEWVGRIKSKNEGGTIDYAAPVKGRFTISRDDSKNTLYLQMNSLKTEDTAVYYCNTDFLWFGELPFDYWGQGTLVTVSS

SEQ ID NO: 822 - protein, 118 aa, synthetic construct
EVQVVESGGGLVQPGGSLRLSCAASGFTFSSYAMTWVRQAPGKGLEWVSLISGSGGLTKYADSVKGRFTISRDNSKKTLYLQMNSLRAEDTAAYYCVKDEGLTTGEYWGQGTLVTVSS

SEQ ID NO: 823 - protein, 125 aa, synthetic construct
QVQLVQSGTEVKKPGASVKVSCKASGYTFTNYDINWVRQATGQGLEWVGWIHPNTGKTGYAQKFQGRVTMTRDISIGTAYMELNSLRSEDTAVYYCARESYDLWSGHSFNWFDPWGQGTLVTVSS

SEQ ID NO: 824 - protein, 122 aa, synthetic construct
QVQLQESGPGLVKPSQTLSLTCTVSGGSISSGGYYWSWIRQYPGKGLEWIGHIYYSGSTKYNPSLKSRLTISVDTSKNQFSLKLSSVTAADTAVYYCARDNWGSGWYYMDVWGKGTTVTVSS

SEQ ID NO: 825 - protein, 113 aa, synthetic construct
DIVMTQSPDSLAVSLGERATINCKSSQSLLYSSNNKNYLAWYQQKPGQPPKLLFYWTSTRESGVPDRFGASGSGTDFTLTISSLQVEDVAIYFCLQYSNTPYSFGQGTKLEIK

SEQ ID NO: 826 - protein, 107 aa, synthetic construct
DIQMTQSPATLSASVGDRVTITCRASQSISTWLVWYQQKPGKAPKLLIYKASSLESGVPSRFSGSGSGTEFTLTISSLQPDDFATYYCQQHISYPYTFGQGTRLEIK

SEQ ID NO: 827 - protein, 113 aa, synthetic construct
DIVMTQSPDSLAVSLGERATINCKSSQSVLYSSNNKNYFAWYQQKPGQPPRLLIYRASTRESGVPDRFSGSGSGTDFTLTISSLQAEDVAVYYCQQYYTTPLTFGGGTKVEIR

SEQ ID NO: 828 - protein, 107 aa, synthetic construct
DNQMTQSPSSLSASVGDRVTITCRASQGISNHLAWYQQKPGKVPDLLIYAASTLQSGVPSRFSGSGSGTDFTLTISSLQPEDVANYYCQKLNSAPLTFGGGTKVEIK

SEQ ID NO: 829 - protein, 107 aa, synthetic construct; identical to SEQ ID NO: 512

SEQ ID NO: 830 - protein, 107 aa, synthetic construct
DIQLTQSPSFLSASVGDRVIITCRASQGITSYLAWYQRKPGKAPKLLIYGATTLQSGVPSRFSGSGSGTEFTLTISSLQPEDFATYYCQQLQSYPYTFGQGTKLEIK

SEQ ID NO: 831 - protein, 107 aa, synthetic construct
EIVLTQSPATLSLSPGERATLSCRASQSVSNYFAWYRQKPGQAPRLLIYDASNRATGIPARFSGSGSGTDFTLTISSLEPEDFAVYYCQHRSNWPLTFGGGTKVEIK

SEQ ID NO: 832 - protein, 127 aa, synthetic construct
EVQLVESGGGLVQPGGSLRLSCAASGFTFSNYAMNWVRQAPGKGLEWVSTISGSGGATRYADSVKGRFTISRDNSRNTVYLQMNSLRVEDTAVFYCTKDRLIMATVRGPYYYGMDVWGQGTTVTVSS

SEQ ID NO: 833 - protein, 107 aa, synthetic construct
DIQMTQSPSSLSASVGDRVTITCRASQSISSYLNWYQQKPGKAPNLLIYAASSLQSGVPSRFSGSGSETDFTLTISSLQPEDFATYYCQQSHSVSFTFGPGTKVDIK

SEQ ID NO: 834 - protein, 120 aa, synthetic construct
EVQLLESGGGLVQPGGSLRLSCAASGFTFNSYAMSWVRQAPGKGLEWVSIISGSGGFTYYADSVKGRFTISRDNSRTTLYLQMNSLRAEDTAVYYCAKDRLVAPGTFDYWGQGALVTVSS

SEQ ID NO: 835 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPYTFGQGTKLEIK

SEQ ID NO: 836 - protein, 120 aa, synthetic construct
QVQLVESGGGVVQPGRSLRLSCAASGFTFSNFGMHWVRQAPGKGLEWVAAIWYDGHDKYYSYYVKGRFTISRDNSKNTLFLQMNSLRAEDTAVYYCARDSSSWYRYFDYWGQGTLVTVSS

SEQ ID NO: 837 - protein, 107 aa, synthetic construct
EIVLTQSPGTLSLSPGERATLSCRASQSVSSNYLAWYQQKPGQAPRLLIYGASSRATGIPDRFSGSGSGTDFTLTISRLEPEDFAVYYCQQYGSSFTFGPGTKVDIK

SEQ ID NO: 838 - protein, 120 aa, synthetic construct
QKQLVEFGGGVVQPGRSLRLSCAASGFTFSNYGMHWVRQAPGKGLWVAVIWNDGSNKYYVDSVKGRFIISRDNSKNTLYLQMNSLRAEDTAVYYCARDRMGIYYYGMDVWGQGTTVTVSS

SEQ ID NO: 839 - protein, 107 aa, synthetic construct
EIVLTQSPATLSLSPGERATLSCRASQGVSRYLAWYQQKPGQAPRLLIYDASNRATGIPARVSGSGPGTDFTLTISSLEPEDFAVDYCQQRSNWQYTFGQGTKLEIK

SEQ ID NO: 840 - protein, 7 aa, synthetic construct
GFTFSNA

SEQ ID NO: 841 - protein, 118 aa, synthetic construct
EVRLVESGGGLVQPGGSLRLSCAASGFTFSSYAMTWVRQAPGKGLEWVSLISGSGGLTKYADSVKGRFTISRDNSKKTLYLQMNSLRAEDTAVYYCAKDEGLTTGEYWGQGTLVTVSS

SEQ ID NO: 842 - protein, 107 aa, synthetic construct
DIQMTQSPSSLSASVGDRVTITCRASQGIRNDLGWYQQKPGKAPKRLIYATSSFQSGVPSRFSGSGSGTEFTLTISSLQPEDFATYYCLQHNSYPFTFGPGTKVDVK

SEQ ID NO: 843 - protein, 509 aa, unidentified
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 844 - protein, 474 aa, unidentified
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 845 - protein, 447 aa, synthetic construct [sequence omitted]

SEQ ID NO: 846 - protein, 446 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIF

SEQ ID NO: 847 - protein, 445 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQI

SEQ ID NO: 848 - protein, 444 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQ

SEQ ID NO: 849 - protein, 443 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEP

SEQ ID NO: 850 - protein, 442 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEE

SEQ ID NO: 851 - protein, 432 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDA

SEQ ID NO: 852 - protein, 448 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 853 - DNA, 1446 bp, synthetic construct
  source 1..1446: mol_type = other DNA
atgggagtgctaaaattcaagcacatctttttcagaagctttgttaaatcaagtggagtatcccagatagttttcaccttccttctgattccatgttgcttgactctgaatttcagagcacctcctgttattccaaatgtgcctttcctctgggcctggaatgccccaagtgaattttgtcttggaaaatttgatgagccactagatatgagcctcttctctttcataggaagcccccgaataaacgccaccgggcaaggtgttacaatattttatgttgatagacttggctactatccttacatagattcaatcacaggagtaactgtgaatggaggaatcccccagaagatttccttacaagaccatctggacaaagctaagaaagacattacattttatatgccagtagacaatttgggaatggctgttattgactgggaagaatggagacccacttgggcaagaaactggaaacctaaagatgtttacaagaataggtctattgaattggttcagcaacaaaatgtacaacttagtctcacagaggccactgagaaagcaaaacaagaatttgaaaaggcagggaaggatttcctggtagagactataaaattgggaaaattacttcggccaaatcacttgtggggttattatctttttccggattgttacaaccatcactataagaaacccggttacaatggaagttgcttcaatgtagaaataaaaagaaatgatgatctcagctggttgtggaatgaaagcactgctctttacccatccatttatttgaacactcagcagtctcctgtagctgctacactctatgtgcgcaatcgagttcgggaagccatcagagtttccaaaatacctgatgcaaaaagtccacttccggtttttgcatatacccgcatagtttttactgatcaagttttgaaattcctttctcaagatgaacttgtgtatacatttggcgaaactgttgctctgggtgcttctggaattgtaatatggggaaccctcagtataatgcgaagtatgaaatcttgcttgctcctagacaattacatggagactatactgaatccttacataatcaacgtcacactagcagccaaaatgtgtagccaagtgctttgccaggagcaaggagtgtgtataaggaaaaactggaattcaagtgactatcttcacctcaacccagataattttgctattcaacttgagaaaggtggaaagttcacagtacgtggaaaaccgacacttgaagacctggagcaattttctgaaaaattttattgcagctgttatagcaccttgagttgtaaggagaaagctgatgtaaaagacactgatgctgttgatgtgtgtattgctgatggtgtctgtatagatgcttttctaaaacctcccatggagacagaagaacctcaaattttctac

SEQ ID NO: 854 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLGPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 855 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPFILNVTSGALLCSQALCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 856 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLGPFILNVTSGALLCSQALCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 857 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWVSWENTRTKESCQAIKEYMDTTLGPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 858 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 859 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWVSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 860 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSNTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 861 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 862 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 863 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPFILNVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 864 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPFILNVTSGAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 865 - protein, 453 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 866 - protein, 430 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCI

SEQ ID NO: 867 - protein, 433 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 868 - protein, 436 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKP

SEQ ID NO: 869 - protein, 452 aa, synthetic construct
FRGPLLPNRPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 870 - protein, 452 aa, synthetic construct
FRGPLLPNRPFTTVWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 871 - protein, 433 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLK

SEQ ID NO: 872 - protein, 431 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 873 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 874 - protein, 451 aa, synthetic construct
APPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 875 - protein, 449 aa, synthetic construct
PVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 876 - protein, 432 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDA

SEQ ID NO: 877 - protein, 429 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVC

SEQ ID NO: 878 - protein, 425 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGPTLEDLEQFSEKFYCSCYSTLSCKEKADVKDTDAVDVCIAD

SEQ ID NO: 879 - protein, 423 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVC

SEQ ID NO: 880 - protein, 420 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAV

SEQ ID NO: 881 - protein, 433 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLK

SEQ ID NO: 882 - protein, 435 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPP

SEQ ID NO: 883 - protein, 436 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPM

SEQ ID NO: 884 - protein, 437 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPME

SEQ ID NO: 885 - protein, 438 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMET

SEQ ID NO: 886 - protein, 439 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETE

SEQ ID NO: 887 - protein, 454 aa, synthetic construct
NFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 888 - protein, 452 aa, synthetic construct
RAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 889 - protein, 450 aa, synthetic construct
PPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSITRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 890 - protein, 428 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCI

SEQ ID NO: 891 - protein, 429 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCID

SEQ ID NO: 892 - protein, 430 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDA

SEQ ID NO: 893 - protein, 431 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 894 - protein, 441 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEP

SEQ ID NO: 895 - protein, 443 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQI

SEQ ID NO: 896 - protein, 445 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 897 - protein, 447 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNA

SEQ ID NO: 898 - protein, 449 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASP

SEQ ID NO: 899 - protein, 451 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPST

SEQ ID NO: 900 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGGWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 901 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGAWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 902 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGCWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 903 - protein, 455 aa, synthetic construct
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGDWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLS

SEQ ID NO: 904 - protein, 431 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSNTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 905 - protein, 431 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 906 - protein, 431 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGAWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 907 - protein, 431 aa, synthetic construct
FRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGGWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 908 - protein, 431 aa, synthetic construct
FRGPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGSWENTRTKESCQAIKEYMDTTLNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAF

SEQ ID NO: 909 - protein, 431 aa, synthetic construct; identical to SEQ ID NO: 908

SEQ ID NO: 910 - protein, 8 aa, synthetic construct
ISGSGGAT

SEQ ID NO: 911 - protein, 20 aa, synthetic construct
TKDRLIMATVRGPYYYGMDV

SEQ ID NO: 912 - protein, 9 aa, synthetic construct
QQSHSVSFT

SEQ ID NO: 913 - protein, 330 aa, Homo sapiens [sequence omitted]

SEQ ID NO: 914 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 915 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 916 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 917 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 918 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 919 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 920 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 921 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 922 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 923 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 924 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 925 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 926 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 927 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 928 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 929 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 930 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 931 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 932 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 933 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 934 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 935 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 936 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 937 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 938 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 939 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 940 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 941 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 942 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 943 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 944 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 945 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 946 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 947 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 948 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 949 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 950 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 951 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 952 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 953 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEELGGPSVFLFPPKPKDTLMISRTPEVTCVVVDVSQEDPEVQFNWYVDGVEVHNAKTKPREEQFNSTYRVVSVLTVDHHDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHSHYTQKSLSLSLGK

SEQ ID NO: 954 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLYITREPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 955 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHSHYTQKSLSLSPGK

SEQ ID NO: 956 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVDHHDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHSHYTQKSLSLSPGK

SEQ ID NO: 957 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 958 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 959 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 960 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 961 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 962 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 963 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHAHYTQKSLSLSPGK

SEQ ID NO: 964 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 965 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 966 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 967 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 968 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 969 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 970 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTQKSLSLSPGK

SEQ ID NO: 971 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 972 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 973 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 974 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 975 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 976 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 977 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHWHYTQKSLSLSPGK

SEQ ID NO: 978 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 979 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 980 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 981 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 982 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 983 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLQVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 984 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLQVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 985 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 986 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVHNAKTKPREEQYASTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 987 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 988 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 989 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 990 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVHNAKTKPREEQYNSTYRVVSVLWVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 991 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVHNAKTKPREEQYNSTYRVVSVLWVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 992 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 993 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 994 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYISRDPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 995 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 996 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 997 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLYISRDPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 998 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYISRDPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 999 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1000 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1001 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1002 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1003 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1004 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLQVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1005 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLQVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1006 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1007 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVDVSHEDPEVKFNWYVDGVEVDNAKTKPREEQYASTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1008 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRDPEVTCVVVAVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1009 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1010 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1011 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRDPEVTCVVVDVSHEDPEVKFNWYVDGVEVDNAKTKPREEQYNSTYRVVSVLRVLHVDWLNGKEYKCKVSNKALPAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1012 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRDPEVTCVVVDVSHEDPEVKFNWYVDGVEVDNAKTKPREEQYNSTYRVVSVLRVLHVDWLNGKEYKCKVSNKALGAPIEKTISKAKGQPREPQVYTLPPSRDELTKNQVSLTCLVKGFYPSDIVVEWESNGQPENNYKTTPPVLDSDGSFFLYSKLTVDKSRWQQGNVFSCSVMHEALHNHYTQKSLSLSPGK

SEQ ID NO: 1013 - protein, 448 aa, synthetic construct
EVQVVESGGGLVQPGGSLRLSCAASGFTFSNYAMTWVRQSPGKGLEWVSLISGSGGLTKYADSVKGRFTISRDNSKKTLFLQMNSLRAEDTAAYYCVKDEGLTTGEYWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1014 - protein, 447 aa, synthetic construct
EVQVVESGGGLVQPGGSLRLSCAASGFTFSNYAMTWVRQSPGKGLEWVSLISGSGGLTKYADSVKGRFTISRDNSKKTLFLQMNSLRAEDTAAYYCVKDEGLTTGEYWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1015 - protein, 214 aa, synthetic construct
DIQMTQSPSSLSASVGDRVTITCRASQDIRNDLAWYQQKPGKAPKRLIYASSSLQSGVPSRFSGSGSGTEFTLTISSLQPEDFATYYCLQHNNYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1016 - protein, 5 aa, Homo sapiens
SRDEL

SEQ ID NO: 1017 - protein, 5 aa, Homo sapiens
SREEM

SEQ ID NO: 1018 - protein, 454 aa, synthetic construct
EVQLVESGGGLVQPGGSLRLSCAASGFTFSNYAMNWVRQAPGKGLEWVSTISGSGGATRYADSVKGRFTISRDNSRNTVYLQMNSLRVEDTAVFYCTKDRLIMATVRGPYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 1019 - protein, 214 aa, synthetic construct
DIQMTQSPSSLSASVGDRVTITCRASQSISSYLNWYQQKPGKAPNLLIYAASSLQSGVPSRFSGSGSETDFTLTISSLQPEDFATYYCQQSHSVSFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC